SEQUENCE LISTING (89 sequences)

SEQ ID NO: 1 - protein, 9 aa, Homo sapiens
SLLQHLIGL

SEQ ID NO: 2 - DNA, 825 bp, synthetic construct
  source 1..825: mol_type = other DNA
atggcctgtcctggcttcctgtgggcccttgtgatcagcacttgcctggaattcagcatggctcagacagtcacccagtctcagcccgaaatgagcgtccaagaggctgaaaccgtgactctgtcttgtacctacgacacctccgagagcgattactacctcttttggtataagcaaccgccgtccaggcaaatgatcctcgtgatccggcaagaagcttacaaacagcagaatgctaccgaaaaccggttctccgtcaattttcagaaagccgctaagagctttagcctgaaaatctccgactctcagctcggcgacgctgctatgtatttctgtgcctaccgcaaaacttcttacgataaagtcatttttgggccagggacaagcttatcagtcattccaaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagc

SEQ ID NO: 3 - protein, 275 aa, synthetic construct
MACPGFLWALVISTCLEFSMAQTVTQSQPEMSVQEAETVTLSCTYDTSESDYYLFWYKQPPSRQMILVIRQEAYKQQNATENRFSVNFQKAAKSFSLKISDSQLGDAAMYFCAYRKTSYDKVIFGPGTSLSVIPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 4 - DNA, 966 bp, synthetic construct
  source 1..966: mol_type = other DNA
atgctgagccccgacctgcctgacagcgcttggaataccagactcctgtgcagagtgatgctgtgcctgcttggagctggaagtgtggctgctggtgtcattcagtccccaaggcacctgatcaaagagaagagagagacagccactctgaagtgctaccccattcctagacacgacacggtctattggtatcagcaaggacctggacaggaccctcagttcctgatcagcttctacgagaagatgcagagcgacaagggcagcatccccgacagattttctgcccagcagttcagcgactaccacagcgagctgaacatgagcagcctggaactgggcgatagcgccctgtacttctgtgcctcttctttcgcacgcctggaaggtcgcgataatgaacaattttttgggccagggacacggctcaccgtgctagaggacctgaacaaggtgttcccacccgaggtcgctgtgtttgagccatcagaagcagagatctcccacacccaaaaggccacactggtgtgcctggccacaggcttcttccctgaccacgtggagctgagctggtgggtgaatgggaaggaggtgcacagtggggtcagcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttc

SEQ ID NO: 5 - protein, 322 aa, synthetic construct
MLSPDLPDSAWNTRLLCRVMLCLLGAGSVAAGVIQSPRHLIKEKRETATLKCYPIPRHDTVYWYQQGPGQDPQFLISFYEKMQSDKGSIPDRFSAQQFSDYHSELNMSSLELGDSALYFCASSFARLEGRDNEQFFGPGTRLTVLEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 6 - DNA, 825 bp, synthetic construct
  source 1..825: mol_type = other DNA
atggcctgtcctggcttcctgtgggcccttgtgatcagcacttgcctggaattcagcatggctcagacagtcacccagtctcagcccgaaatgagcgtccaagaggctgaaaccgtgactctgtcttgtacctacgacacctccgagagcgattactacctcttttggtataagcaaccgccgtccaggcaaatgatcctcgtgatccggcaagaagcttacaaacagcagaatgctaccgaaaaccggttctccgtcaattttcagaaagccgctaagagctttagcctgaaaatctccgactctcagctcggcgacgctgctatgtatttctgtgcctaccgcaaaacttcttacgataaagtcatttttgggccagggacaagcttatcagtcattccaaacatccagaaccccgaccccgccgtgtaccagctgagggactccaagtccagcgacaagagcgtgtgtctgtttacggacttcgacagccagaccaacgtgagtcaaagcaaggacagcgacgtctacataacggataagaccgtgctggacatgcggagcatggacttcaagagcaacagcgccgtggcctggtccaacaagagcgacttcgcctgcgccaacgccttcaacaacagcatcatccccgaggacaccttcttccccagcagcgacgtgccctgcgacgtgaaactggtggagaagtccttcgagacagacaccaatctgaactttcagaacctgctggtgatcgtgctgcggattctgctgctgaaagtggccggcttcaatctgctgatgaccctgcggctgtggagcagc

SEQ ID NO: 7 - protein, 275 aa, synthetic construct
MACPGFLWALVISTCLEFSMAQTVTQSQPEMSVQEAETVTLSCTYDTSESDYYLFWYKQPPSRQMILVIRQEAYKQQNATENRFSVNFQKAAKSFSLKISDSQLGDAAMYFCAYRKTSYDKVIFGPGTSLSVIPNIQNPDPAVYQLRDSKSSDKSVCLFTDFDSQTNVSQSKDSDVYITDKTVLDMRSMDFKSNSAVAWSNKSDFACANAFNNSIIPEDTFFPSSDVPCDVKLVEKSFETDTNLNFQNLLVIVLRILLLKVAGFNLLMTLRLWSS

SEQ ID NO: 8 - DNA, 975 bp, synthetic construct
  source 1..975: mol_type = other DNA
atgctgagccccgacctgcctgacagcgcttggaataccagactcctgtgcagagtgatgctgtgcctgcttggagctggaagtgtggctgctggtgtcattcagtccccaaggcacctgatcaaagagaagagagagacagccactctgaagtgctaccccattcctagacacgacacggtctattggtatcagcaaggacctggacaggaccctcagttcctgatcagcttctacgagaagatgcagagcgacaagggcagcatccccgacagattttctgcccagcagttcagcgactaccacagcgagctgaacatgagcagcctggaactgggcgatagcgccctgtacttctgtgcctcttctttcgcacgcctggaaggtcgcgataatgaacaattttttgggccagggacacggctcaccgtgctagaagatctgaacaaggtgttccctccagaggtggccgtgttcgagccttctaaggccgagatcgcccacacacaaaaagccaccctcgtgtgcctggccaccggctttttccccgaccacgtggaactgtcttggtgggtcaacggcaaagaggtgcactccggcgtgtcaacggatccccagcctctgaaagaacagcctgccctgaacgacagccggtactgcctgagctccagactgagagtgtccgccaccttctggcagaacccccggaaccacttcagatgccaggtgcagttttacggcctgagcgagaacgacgagtggacccaggacagagccaagcccgtgacacaaatcgtgtctgccgaagcctggggaagagccgattgcggcatcaccagcgcctcctatcaccagggcgtgctgagcgccacaatcctgtacgaaatcctgctgggcaaggccaccctgtacgccgtgctggtgtctgctctggtgctgatggccatggtcaagcggaaggactttggcagcggc

SEQ ID NO: 9 - protein, 325 aa, synthetic construct
MLSPDLPDSAWNTRLLCRVMLCLLGAGSVAAGVIQSPRHLIKEKRETATLKCYPIPRHDTVYWYQQGPGQDPQFLISFYEKMQSDKGSIPDRFSAQQFSDYHSELNMSSLELGDSALYFCASSFARLEGRDNEQFFGPGTRLTVLEDLNKVFPPEVAVFEPSKAEIAHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGITSASYHQGVLSATILYEILLGKATLYAVLVSALVLMAMVKRKDFGSG

SEQ ID NO: 10 - DNA, 1881 bp, synthetic construct
  source 1..1881: mol_type = other DNA
atgctgagccccgacctgcctgacagcgcttggaataccagactcctgtgcagagtgatgctgtgcctgcttggagctggaagtgtggctgctggtgtcattcagtccccaaggcacctgatcaaagagaagagagagacagccactctgaagtgctaccccattcctagacacgacacggtctattggtatcagcaaggacctggacaggaccctcagttcctgatcagcttctacgagaagatgcagagcgacaagggcagcatccccgacagattttctgcccagcagttcagcgactaccacagcgagctgaacatgagcagcctggaactgggcgatagcgccctgtacttctgtgcctcttctttcgcacgcctggaaggtcgcgataatgaacaattttttgggccagggacacggctcaccgtgctagaagatctgaacaaggtgttccctccagaggtggccgtgttcgagccttctaaggccgagatcgcccacacacaaaaagccaccctcgtgtgcctggccaccggctttttccccgaccacgtggaactgtcttggtgggtcaacggcaaagaggtgcactccggcgtgtcaacggatccccagcctctgaaagaacagcctgccctgaacgacagccggtactgcctgagctccagactgagagtgtccgccaccttctggcagaacccccggaaccacttcagatgccaggtgcagttttacggcctgagcgagaacgacgagtggacccaggacagagccaagcccgtgacacaaatcgtgtctgccgaagcctggggaagagccgattgcggcatcaccagcgcctcctatcaccagggcgtgctgagcgccacaatcctgtacgaaatcctgctgggcaaggccaccctgtacgccgtgctggtgtctgctctggtgctgatggccatggtcaagcggaaggactttggcagcggcagagccaaaaggtccgggagcggtgcgacaaactttagcctgttgaaacaagccggcgacgttgaagagaaccccggacctatggcctgtcctggcttcctgtgggcccttgtgatcagcacttgcctggaattcagcatggctcagacagtcacccagtctcagcccgaaatgagcgtccaagaggctgaaaccgtgactctgtcttgtacctacgacacctccgagagcgattactacctcttttggtataagcaaccgccgtccaggcaaatgatcctcgtgatccggcaagaagcttacaaacagcagaatgctaccgaaaaccggttctccgtcaattttcagaaagccgctaagagctttagcctgaaaatctccgactctcagctcggcgacgctgctatgtatttctgtgcctaccgcaaaacttcttacgataaagtcatttttgggccagggacaagcttatcagtcattccaaacatccagaaccccgaccccgccgtgtaccagctgagggactccaagtccagcgacaagagcgtgtgtctgtttacggacttcgacagccagaccaacgtgagtcaaagcaaggacagcgacgtctacataacggataagaccgtgctggacatgcggagcatggacttcaagagcaacagcgccgtggcctggtccaacaagagcgacttcgcctgcgccaacgccttcaacaacagcatcatccccgaggacaccttcttccccagcagcgacgtgccctgcgacgtgaaactggtggagaagtccttcgagacagacaccaatctgaactttcagaacctgctggtgatcgtgctgcggattctgctgctgaaagtggccggcttcaatctgctgatgaccctgcggctgtggagcagc

SEQ ID NO: 11 - protein, 627 aa, synthetic construct
MLSPDLPDSAWNTRLLCRVMLCLLGAGSVAAGVIQSPRHLIKEKRETATLKCYPIPRHDTVYWYQQGPGQDPQFLISFYEKMQSDKGSIPDRFSAQQFSDYHSELNMSSLELGDSALYFCASSFARLEGRDNEQFFGPGTRLTVLEDLNKVFPPEVAVFEPSKAEIAHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGITSASYHQGVLSATILYEILLGKATLYAVLVSALVLMAMVKRKDFGSGRAKRSGSGATNFSLLKQAGDVEENPGPMACPGFLWALVISTCLEFSMAQTVTQSQPEMSVQEAETVTLSCTYDTSESDYYLFWYKQPPSRQMILVIRQEAYKQQNATENRFSVNFQKAAKSFSLKISDSQLGDAAMYFCAYRKTSYDKVIFGPGTSLSVIPNIQNPDPAVYQLRDSKSSDKSVCLFTDFDSQTNVSQSKDSDVYITDKTVLDMRSMDFKSNSAVAWSNKSDFACANAFNNSIIPEDTFFPSSDVPCDVKLVEKSFETDTNLNFQNLLVIVLRILLLKVAGFNLLMTLRLWSS

SEQ ID NO: 12 - DNA, 816 bp, synthetic construct
  source 1..816: mol_type = other DNA
atggaaaccctgctgaaggtgctgtctggcaccctgctgtggcagctgacatgggtccgatctcagcagcctgtgcagtctcctcaggccgtgattctgagagaaggcgaggacgccgtgatcaactgcagcagctctaaggccctgtacagcgtgcactggtacaggcagaaacacggcgaggccccagtgtttctgatgattctgctgaaaggcggcgagcagaagggccacgataagatctccgccagcttcaacgagaagaagcagcagtccagcctgtacctgacagccagccagctgagctacagcggcacctatttctgtggcacagaaggtactggtgactacaagctctcttttggagccggaaccacagtaactgtaagagcaaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagc

SEQ ID NO: 13 - protein, 272 aa, synthetic construct
METLLKVLSGTLLWQLTWVRSQQPVQSPQAVILREGEDAVINCSSSKALYSVHWYRQKHGEAPVFLMILLKGGEQKGHDKISASFNEKKQQSSLYLTASQLSYSGTYFCGTEGTGDYKLSFGAGTTVTVRANIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 14 - DNA, 927 bp, synthetic construct
  source 1..927: mol_type = other DNA
atgggacctcagctgctgggatatgtggtgctgtgtctgctcggagctggacccctggaagctcaagtgacacagaaccccagatacctgatcaccgtgaccggcaaaaagctgaccgtgacctgtagccagaacatgaaccacgagtacatgagctggtatcggcaagaccctggcctggggctgagacagatctactatagcatgaacgtggaagtgaccgacaaaggcgacgtgcccgagggctataaggtgtcccggaaagagaagcggaactttccactgatcctggaatccccatctcctaaccagaccagcctgtatttttgcgctagttctgccgggaccggggggcatgagcaatacttcgggccgggcaccaggctcacggtcacagaggacctgaacaaggtgttcccacccgaggtcgctgtgtttgagccatcagaagcagagatctcccacacccaaaaggccacactggtgtgcctggccacaggcttcttccctgaccacgtggagctgagctggtgggtgaatgggaaggaggtgcacagtggggtcagcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttc

SEQ ID NO: 15 - protein, 309 aa, synthetic construct
MGPQLLGYVVLCLLGAGPLEAQVTQNPRYLITVTGKKLTVTCSQNMNHEYMSWYRQDPGLGLRQIYYSMNVEVTDKGDVPEGYKVSRKEKRNFPLILESPSPNQTSLYFCASSAGTGGHEQYFGPGTRLTVTEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 16 - DNA, 813 bp, synthetic construct
  source 1..813: mol_type = other DNA
atggaaaccctgctgaaggtgctgtctggcaccctgctgtggcagctgacatgggtccgatctcagcagcctgtgcagtctcctcaggccgtgattctgagagaaggcgaggacgccgtgatcaactgcagcagctctaaggccctgtacagcgtgcactggtacaggcagaaacacggcgaggccccagtgtttctgatgattctgctgaaaggcggcgagcagaagggccacgataagatctccgccagcttcaacgagaagaagcagcagtccagcctgtacctgacagccagccagctgagctacagcggcacctatttctgtggcacagaaggtactggtgactacaagctctcttttggagccggaaccacagtaactgtaagagcaaacatccagaaccccgaccccgccgtgtaccagctgagggactccaagtccagcgacaagagcgtgtgtctgtttacggacttcgacagccagaccaacgtgagtcaaagcaaggacagcgacgtctacataacggataagaccgtgctggacatgcggagcatggacttcaagagcaacagcgccgtggcctggtccaacaagagcgacttcgcctgcgccaacgccttcaacaacagcatcatccccgaggacaccttcttccccagcagcgacgtgccctgcgacgtgaaactggtggagaagtccttcgagacagacaccaatctgaactttcagaacctgctggtgatcgtgctgcggattctgctgctgaaagtggccggcttcaatctgctgatgaccctgcggctgtggagc

SEQ ID NO: 17 - protein, 271 aa, synthetic construct
METLLKVLSGTLLWQLTWVRSQQPVQSPQAVILREGEDAVINCSSSKALYSVHWYRQKHGEAPVFLMILLKGGEQKGHDKISASFNEKKQQSSLYLTASQLSYSGTYFCGTEGTGDYKLSFGAGTTVTVRANIQNPDPAVYQLRDSKSSDKSVCLFTDFDSQTNVSQSKDSDVYITDKTVLDMRSMDFKSNSAVAWSNKSDFACANAFNNSIIPEDTFFPSSDVPCDVKLVEKSFETDTNLNFQNLLVIVLRILLLKVAGFNLLMTLRLWS

SEQ ID NO: 18 - DNA, 960 bp, synthetic construct
  source 1..960: mol_type = other DNA
atgggacctcagctgctgggatatgtggtgctgtgtctgctcggagctggacccctggaagctcaagtgacacagaaccccagatacctgatcaccgtgaccggcaaaaagctgaccgtgacctgtagccagaacatgaaccacgagtacatgagctggtatcggcaagaccctggcctggggctgagacagatctactatagcatgaacgtggaagtgaccgacaaaggcgacgtgcccgagggctataaggtgtcccggaaagagaagcggaactttccactgatcctggaatccccatctcctaaccagaccagcctgtatttttgcgctagttctgccgggaccggggggcatgagcaatacttcgggccgggcaccaggctcacggtcacagaagatctgaacaaggtgttccctccagaggtggccgtgttcgagccttctaaggccgagatcgcccacacacaaaaagccaccctcgtgtgcctggccaccggctttttccccgaccacgtggaactgtcttggtgggtcaacggcaaagaggtgcactccggcgtgtcaacggatccccagcctctgaaagaacagcctgccctgaacgacagccggtactgcctgagctccagactgagagtgtccgccaccttctggcagaacccccggaaccacttcagatgccaggtgcagttttacggcctgagcgagaacgacgagtggacccaggacagagccaagcccgtgacacaaatcgtgtctgccgaagcctggggaagagccgattgcggcatcaccagcgcctcctatcaccagggcgtgctgagcgccacaatcctgtacgaaatcctgctgggcaaggccaccctgtacgccgtgctggtgtctgctctggtgctgatggccatggtcaagcggaaggactttggcagcggcagagccaaaaggtccgggagcggt

SEQ ID NO: 19 - protein, 320 aa, synthetic construct
MGPQLLGYVVLCLLGAGPLEAQVTQNPRYLITVTGKKLTVTCSQNMNHEYMSWYRQDPGLGLRQIYYSMNVEVTDKGDVPEGYKVSRKEKRNFPLILESPSPNQTSLYFCASSAGTGGHEQYFGPGTRLTVTEDLNKVFPPEVAVFEPSKAEIAHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGITSASYHQGVLSATILYEILLGKATLYAVLVSALVLMAMVKRKDFGSGRAKRSGSG

SEQ ID NO: 20 - DNA, 1830 bp, synthetic construct
  source 1..1830: mol_type = other DNA
atgggacctcagctgctgggatatgtggtgctgtgtctgctcggagctggacccctggaagctcaagtgacacagaaccccagatacctgatcaccgtgaccggcaaaaagctgaccgtgacctgtagccagaacatgaaccacgagtacatgagctggtatcggcaagaccctggcctggggctgagacagatctactatagcatgaacgtggaagtgaccgacaaaggcgacgtgcccgagggctataaggtgtcccggaaagagaagcggaactttccactgatcctggaatccccatctcctaaccagaccagcctgtatttttgcgctagttctgccgggaccggggggcatgagcaatacttcgggccgggcaccaggctcacggtcacagaagatctgaacaaggtgttccctccagaggtggccgtgttcgagccttctaaggccgagatcgcccacacacaaaaagccaccctcgtgtgcctggccaccggctttttccccgaccacgtggaactgtcttggtgggtcaacggcaaagaggtgcactccggcgtgtcaacggatccccagcctctgaaagaacagcctgccctgaacgacagccggtactgcctgagctccagactgagagtgtccgccaccttctggcagaacccccggaaccacttcagatgccaggtgcagttttacggcctgagcgagaacgacgagtggacccaggacagagccaagcccgtgacacaaatcgtgtctgccgaagcctggggaagagccgattgcggcatcaccagcgcctcctatcaccagggcgtgctgagcgccacaatcctgtacgaaatcctgctgggcaaggccaccctgtacgccgtgctggtgtctgctctggtgctgatggccatggtcaagcggaaggactttggcagcggcagagccaaaaggtccgggagcggtgcgacaaactttagcctgttgaaacaagccggcgacgttgaagagaaccccggacctatggaaaccctgctgaaggtgctgtctggcaccctgctgtggcagctgacatgggtccgatctcagcagcctgtgcagtctcctcaggccgtgattctgagagaaggcgaggacgccgtgatcaactgcagcagctctaaggccctgtacagcgtgcactggtacaggcagaaacacggcgaggccccagtgtttctgatgattctgctgaaaggcggcgagcagaagggccacgataagatctccgccagcttcaacgagaagaagcagcagtccagcctgtacctgacagccagccagctgagctacagcggcacctatttctgtggcacagaaggtactggtgactacaagctctcttttggagccggaaccacagtaactgtaagagcaaacatccagaaccccgaccccgccgtgtaccagctgagggactccaagtccagcgacaagagcgtgtgtctgtttacggacttcgacagccagaccaacgtgagtcaaagcaaggacagcgacgtctacataacggataagaccgtgctggacatgcggagcatggacttcaagagcaacagcgccgtggcctggtccaacaagagcgacttcgcctgcgccaacgccttcaacaacagcatcatccccgaggacaccttcttccccagcagcgacgtgccctgcgacgtgaaactggtggagaagtccttcgagacagacaccaatctgaactttcagaacctgctggtgatcgtgctgcggattctgctgctgaaagtggccggcttcaatctgctgatgaccctgcggctgtggagc

SEQ ID NO: 21 - protein, 610 aa, synthetic construct
MGPQLLGYVVLCLLGAGPLEAQVTQNPRYLITVTGKKLTVTCSQNMNHEYMSWYRQDPGLGLRQIYYSMNVEVTDKGDVPEGYKVSRKEKRNFPLILESPSPNQTSLYFCASSAGTGGHEQYFGPGTRLTVTEDLNKVFPPEVAVFEPSKAEIAHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGITSASYHQGVLSATILYEILLGKATLYAVLVSALVLMAMVKRKDFGSGRAKRSGSGATNFSLLKQAGDVEENPGPMETLLKVLSGTLLWQLTWVRSQQPVQSPQAVILREGEDAVINCSSSKALYSVHWYRQKHGEAPVFLMILLKGGEQKGHDKISASFNEKKQQSSLYLTASQLSYSGTYFCGTEGTGDYKLSFGAGTTVTVRANIQNPDPAVYQLRDSKSSDKSVCLFTDFDSQTNVSQSKDSDVYITDKTVLDMRSMDFKSNSAVAWSNKSDFACANAFNNSIIPEDTFFPSSDVPCDVKLVEKSFETDTNLNFQNLLVIVLRILLLKVAGFNLLMTLRLWS

SEQ ID NO: 22 - DNA, 2121 bp, Homo sapiens
  source 1..2121: mol_type = genomic DNA
acagctcccccgcagccagaagccgggcctgcagcgcctcagcaccgctccgggacaccccacccgcttcccaggcgtgacctgtcaacagcaacttcgcggtgtggtgaactctctgaggaaaaaccattttgattattactctcagacgtgcgtggcaacaagtgactgagacctagaaatccaagcgttggaggtcctgaggccagcctaagtcgcttcaaaatggaacgaaggcgtttgtggggttccattcagagccgatacatcagcatgagtgtgtggacaagcccacggagacttgtggagctggcagggcagagcctgctgaaggatgaggccctggccattgccgccctggagttgctgcccagggagctcttcccgccactcttcatggcagcctttgacgggagacacagccagaccctgaaggcaatggtgcaggcctggcccttcacctgcctccctctgggagtgctgatgaagggacaacatcttcacctggagaccttcaaagctgtgcttgatggacttgatgtgctccttgcccaggaggttcgccccaggaggtggaaacttcaagtgctggatttacggaagaactctcatcaggacttctggactgtatggtctggaaacagggccagtctgtactcatttccagagccagaagcagctcagcccatgacaaagaagcgaaaagtagatggtttgagcacagaggcagagcagcccttcattccagtagaggtgctcgtagacctgttcctcaaggaaggtgcctgtgatgaattgttctcctacctcattgagaaagtgaagcgaaagaaaaatgtactacgcctgtgctgtaagaagctgaagatttttgcaatgcccatgcaggatatcaagatgatcctgaaaatggtgcagctggactctattgaagatttggaagtgacttgtacctggaagctacccaccttggcgaaattttctccttacctgggccagatgattaatctgcgtagactcctcctctcccacatccatgcatcttcctacatttccccggagaaggaagagcagtatatcgcccagttcacctctcagttcctcagtctgcagtgcctgcaggctctctatgtggactctttatttttccttagaggccgcctggatcagttgctcaggcacgtgatgaaccccttggaaaccctctcaataactaactgccggctttcggaaggggatgtgatgcatctgtcccagagtcccagcgtcagtcagctaagtgtcctgagtctaagtggggtcatgctgaccgatgtaagtcccgagcccctccaagctctgctggagagagcctctgccaccctccaggacctggtctttgatgagtgtgggatcacggatgatcagctccttgccctcctgccttccctgagccactgctcccagcttacgaccttaagcttctacgggaattccatctccatatctgccctgcagagtctcctgcagcacctcatcgggctgagcaatctgacccacgtgctgtatcctgtccccctggagagttatgaggacatccatggtaccctccacctggagaggcttgcctatctgcatgccaggctcagggagttgctgtgtgagttggggcggcccagcatggtctggcttagtgccaacccctgtcctcactgtggggacagaaccttctatgacccggagcccatcctgtgcccctgtttcatgcctaattagctgggtgcacatatcaaatgcttcattctgcatacttggacactaaagccaggatgtgcatgcatcttgaagcaacaaagcagccacagtttcagacaaatgttcagtgtgagtgaggaaaacatgttcagtgaggaaaaaacattcagacaaatgttcagtgaggaaaaaaaggggaagttgggggtaggcagatgttgacttgaggagttaatgtgatctttggggagatacatcttatagagttagaaatagaatctgaatttctaaagggagattctggcttgggaagtacatgtaggagttaatccctgtgtagactgttgtaaagaaactgttgaaaataaagagaagcaatgtgaagca

SEQ ID NO: 23 - DNA, 2735 bp, Homo sapiens
  source 1..2735: mol_type = genomic DNA
acagctcccccgcagccagaagccgggcctgcagcgcctcagcaccgctccgggacaccccacccgcttcccaggcgtgacctgtcaacagcaacttcgcggtgtggtgaactctctgaggaaaaacgtaagttcgagccctgattcctccgcttccccgcagggtgaccttgggcttgtgcccccagcaccacccctgtcccgggtccctgttttctctctggaaatgggttgaagaccaaagaaaataatgtgcgccacttgggtcaccccgggccgcctgccccggaaaattggccccagttgaggagttgtggctgtaaggatgccttgaaccgaggcggcggtgctcgtggttggagctctccagggtgggtgcgcatttgtaatgcggtggatgctctgggactcggcccctctgaaggtgctgggggttggggacggcccaggcagtggcgtaggcgtcctaggaaggcgggagcagaggcagaaatgtcgctgcaagaccgtagtcagggtccttgaccacaggggtcacttgtgaccaaccacatggtctgttgttcctcctgccccctggttcagcccaggaaacactggtgctcaggtttggagccagagatttgcactgaaagggcgggattgagtcgccagttgtcagtttcctcagcagtatttgcggaggttttcacaggaggccgttgcttcgtaaatattatacatgtattcttctttttggagcattttgattattactctcagacgtgcgtggcaacaagtgactgagacctagaaatccaagcgttggaggtcctgaggccagcctaagtcgcttcaaaatggaacgaaggcgtttgtggggttccattcagagccgatacatcagcatgagtgtgtggacaagcccacggagacttgtggagctggcagggcagagcctgctgaaggatgaggccctggccattgccgccctggagttgctgcccagggagctcttcccgccactcttcatggcagcctttgacgggagacacagccagaccctgaaggcaatggtgcaggcctggcccttcacctgcctccctctgggagtgctgatgaagggacaacatcttcacctggagaccttcaaagctgtgcttgatggacttgatgtgctccttgcccaggaggttcgccccaggaggtggaaacttcaagtgctggatttacggaagaactctcatcaggacttctggactgtatggtctggaaacagggccagtctgtactcatttccagagccagaagcagctcagcccatgacaaagaagcgaaaagtagatggtttgagcacagaggcagagcagcccttcattccagtagaggtgctcgtagacctgttcctcaaggaaggtgcctgtgatgaattgttctcctacctcattgagaaagtgaagcgaaagaaaaatgtactacgcctgtgctgtaagaagctgaagatttttgcaatgcccatgcaggatatcaagatgatcctgaaaatggtgcagctggactctattgaagatttggaagtgacttgtacctggaagctacccaccttggcgaaattttctccttacctgggccagatgattaatctgcgtagactcctcctctcccacatccatgcatcttcctacatttccccggagaaggaagagcagtatatcgcccagttcacctctcagttcctcagtctgcagtgcctgcaggctctctatgtggactctttatttttccttagaggccgcctggatcagttgctcaggcacgtgatgaaccccttggaaaccctctcaataactaactgccggctttcggaaggggatgtgatgcatctgtcccagagtcccagcgtcagtcagctaagtgtcctgagtctaagtggggtcatgctgaccgatgtaagtcccgagcccctccaagctctgctggagagagcctctgccaccctccaggacctggtctttgatgagtgtgggatcacggatgatcagctccttgccctcctgccttccctgagccactgctcccagcttacgaccttaagcttctacgggaattccatctccatatctgccctgcagagtctcctgcagcacctcatcgggctgagcaatctgacccacgtgctgtatcctgtccccctggagagttatgaggacatccatggtaccctccacctggagaggcttgcctatctgcatgccaggctcagggagttgctgtgtgagttggggcggcccagcatggtctggcttagtgccaacccctgtcctcactgtggggacagaaccttctatgacccggagcccatcctgtgcccctgtttcatgcctaattagctgggtgcacatatcaaatgcttcattctgcatacttggacactaaagccaggatgtgcatgcatcttgaagcaacaaagcagccacagtttcagacaaatgttcagtgtgagtgaggaaaacatgttcagtgaggaaaaaacattcagacaaatgttcagtgaggaaaaaaaggggaagttgggggtaggcagatgttgacttgaggagttaatgtgatctttggggagatacatcttatagagttagaaatagaatctgaatttctaaagggagattctggcttgggaagtacatgtaggagttaatccctgtgtagactgttgtaaagaaactgttgaaaataaagagaagcaatgtgaagca

SEQ ID NO: 24 - DNA, 2100 bp, Homo sapiens
  source 1..2100: mol_type = genomic DNA
acagctcccccgcagccagaagccgggcctgcagcgcctcagcaccgctccgggacaccccacccgcttcccaggcgtgacctgtcaacagcaacttcgcggtgtggtgaactctctgaggaaaaacacgtgcgtggcaacaagtgactgagacctagaaatccaagcgttggaggtcctgaggccagcctaagtcgcttcaaaatggaacgaaggcgtttgtggggttccattcagagccgatacatcagcatgagtgtgtggacaagcccacggagacttgtggagctggcagggcagagcctgctgaaggatgaggccctggccattgccgccctggagttgctgcccagggagctcttcccgccactcttcatggcagcctttgacgggagacacagccagaccctgaaggcaatggtgcaggcctggcccttcacctgcctccctctgggagtgctgatgaagggacaacatcttcacctggagaccttcaaagctgtgcttgatggacttgatgtgctccttgcccaggaggttcgccccaggaggtggaaacttcaagtgctggatttacggaagaactctcatcaggacttctggactgtatggtctggaaacagggccagtctgtactcatttccagagccagaagcagctcagcccatgacaaagaagcgaaaagtagatggtttgagcacagaggcagagcagcccttcattccagtagaggtgctcgtagacctgttcctcaaggaaggtgcctgtgatgaattgttctcctacctcattgagaaagtgaagcgaaagaaaaatgtactacgcctgtgctgtaagaagctgaagatttttgcaatgcccatgcaggatatcaagatgatcctgaaaatggtgcagctggactctattgaagatttggaagtgacttgtacctggaagctacccaccttggcgaaattttctccttacctgggccagatgattaatctgcgtagactcctcctctcccacatccatgcatcttcctacatttccccggagaaggaagagcagtatatcgcccagttcacctctcagttcctcagtctgcagtgcctgcaggctctctatgtggactctttatttttccttagaggccgcctggatcagttgctcaggcacgtgatgaaccccttggaaaccctctcaataactaactgccggctttcggaaggggatgtgatgcatctgtcccagagtcccagcgtcagtcagctaagtgtcctgagtctaagtggggtcatgctgaccgatgtaagtcccgagcccctccaagctctgctggagagagcctctgccaccctccaggacctggtctttgatgagtgtgggatcacggatgatcagctccttgccctcctgccttccctgagccactgctcccagcttacgaccttaagcttctacgggaattccatctccatatctgccctgcagagtctcctgcagcacctcatcgggctgagcaatctgacccacgtgctgtatcctgtccccctggagagttatgaggacatccatggtaccctccacctggagaggcttgcctatctgcatgccaggctcagggagttgctgtgtgagttggggcggcccagcatggtctggcttagtgccaacccctgtcctcactgtggggacagaaccttctatgacccggagcccatcctgtgcccctgtttcatgcctaattagctgggtgcacatatcaaatgcttcattctgcatacttggacactaaagccaggatgtgcatgcatcttgaagcaacaaagcagccacagtttcagacaaatgttcagtgtgagtgaggaaaacatgttcagtgaggaaaaaacattcagacaaatgttcagtgaggaaaaaaaggggaagttgggggtaggcagatgttgacttgaggagttaatgtgatctttggggagatacatcttatagagttagaaatagaatctgaatttctaaagggagattctggcttgggaagtacatgtaggagttaatccctgtgtagactgttgtaaagaaactgttgaaaataaagagaagcaatgtgaagca

SEQ ID NO: 25 - DNA, 2325 bp, Homo sapiens
  source 1..2325: mol_type = genomic DNA
acagctcccccgcagccagaagccgggcctgcagcgcctcagcaccgctccgggacaccccacccgcttcccaggcgtgacctgtcaacaggtctgtattggcgacaaaaggagcagccctgaatgtagggaaagcagggcggagtcctctgcaggctcgggggaggggaggggcgtgaatgcgtggatttctgtggagagtggaaacacggggagtcgaggggagcatgcgcgggcctcagaaagttctgggaaaccgactcccgggagcagggaggaacgcgcgctccagagacaacttcgcggtgtggtgaactctctgaggaaaaaccattttgattattactctcagacgtgcgtggcaacaagtgactgagacctagaaatccaagcgttggaggtcctgaggccagcctaagtcgcttcaaaatggaacgaaggcgtttgtggggttccattcagagccgatacatcagcatgagtgtgtggacaagcccacggagacttgtggagctggcagggcagagcctgctgaaggatgaggccctggccattgccgccctggagttgctgcccagggagctcttcccgccactcttcatggcagcctttgacgggagacacagccagaccctgaaggcaatggtgcaggcctggcccttcacctgcctccctctgggagtgctgatgaagggacaacatcttcacctggagaccttcaaagctgtgcttgatggacttgatgtgctccttgcccaggaggttcgccccaggaggtggaaacttcaagtgctggatttacggaagaactctcatcaggacttctggactgtatggtctggaaacagggccagtctgtactcatttccagagccagaagcagctcagcccatgacaaagaagcgaaaagtagatggtttgagcacagaggcagagcagcccttcattccagtagaggtgctcgtagacctgttcctcaaggaaggtgcctgtgatgaattgttctcctacctcattgagaaagtgaagcgaaagaaaaatgtactacgcctgtgctgtaagaagctgaagatttttgcaatgcccatgcaggatatcaagatgatcctgaaaatggtgcagctggactctattgaagatttggaagtgacttgtacctggaagctacccaccttggcgaaattttctccttacctgggccagatgattaatctgcgtagactcctcctctcccacatccatgcatcttcctacatttccccggagaaggaagagcagtatatcgcccagttcacctctcagttcctcagtctgcagtgcctgcaggctctctatgtggactctttatttttccttagaggccgcctggatcagttgctcaggcacgtgatgaaccccttggaaaccctctcaataactaactgccggctttcggaaggggatgtgatgcatctgtcccagagtcccagcgtcagtcagctaagtgtcctgagtctaagtggggtcatgctgaccgatgtaagtcccgagcccctccaagctctgctggagagagcctctgccaccctccaggacctggtctttgatgagtgtgggatcacggatgatcagctccttgccctcctgccttccctgagccactgctcccagcttacgaccttaagcttctacgggaattccatctccatatctgccctgcagagtctcctgcagcacctcatcgggctgagcaatctgacccacgtgctgtatcctgtccccctggagagttatgaggacatccatggtaccctccacctggagaggcttgcctatctgcatgccaggctcagggagttgctgtgtgagttggggcggcccagcatggtctggcttagtgccaacccctgtcctcactgtggggacagaaccttctatgacccggagcccatcctgtgcccctgtttcatgcctaattagctgggtgcacatatcaaatgcttcattctgcatacttggacactaaagccaggatgtgcatgcatcttgaagcaacaaagcagccacagtttcagacaaatgttcagtgtgagtgaggaaaacatgttcagtgaggaaaaaacattcagacaaatgttcagtgaggaaaaaaaggggaagttgggggtaggcagatgttgacttgaggagttaatgtgatctttggggagatacatcttatagagttagaaatagaatctgaatttctaaagggagattctggcttgggaagtacatgtaggagttaatccctgtgtagactgttgtaaagaaactgttgaaaataaagagaagcaatgtgaagca

SEQ ID NO: 26 - DNA, 2304 bp, Homo sapiens
  source 1..2304: mol_type = genomic DNA
acagctcccccgcagccagaagccgggcctgcagcgcctcagcaccgctccgggacaccccacccgcttcccaggcgtgacctgtcaacaggtctgtattggcgacaaaaggagcagccctgaatgtagggaaagcagggcggagtcctctgcaggctcgggggaggggaggggcgtgaatgcgtggatttctgtggagagtggaaacacggggagtcgaggggagcatgcgcgggcctcagaaagttctgggaaaccgactcccgggagcagggaggaacgcgcgctccagagacaacttcgcggtgtggtgaactctctgaggaaaaacacgtgcgtggcaacaagtgactgagacctagaaatccaagcgttggaggtcctgaggccagcctaagtcgcttcaaaatggaacgaaggcgtttgtggggttccattcagagccgatacatcagcatgagtgtgtggacaagcccacggagacttgtggagctggcagggcagagcctgctgaaggatgaggccctggccattgccgccctggagttgctgcccagggagctcttcccgccactcttcatggcagcctttgacgggagacacagccagaccctgaaggcaatggtgcaggcctggcccttcacctgcctccctctgggagtgctgatgaagggacaacatcttcacctggagaccttcaaagctgtgcttgatggacttgatgtgctccttgcccaggaggttcgccccaggaggtggaaacttcaagtgctggatttacggaagaactctcatcaggacttctggactgtatggtctggaaacagggccagtctgtactcatttccagagccagaagcagctcagcccatgacaaagaagcgaaaagtagatggtttgagcacagaggcagagcagcccttcattccagtagaggtgctcgtagacctgttcctcaaggaaggtgcctgtgatgaattgttctcctacctcattgagaaagtgaagcgaaagaaaaatgtactacgcctgtgctgtaagaagctgaagatttttgcaatgcccatgcaggatatcaagatgatcctgaaaatggtgcagctggactctattgaagatttggaagtgacttgtacctggaagctacccaccttggcgaaattttctccttacctgggccagatgattaatctgcgtagactcctcctctcccacatccatgcatcttcctacatttccccggagaaggaagagcagtatatcgcccagttcacctctcagttcctcagtctgcagtgcctgcaggctctctatgtggactctttatttttccttagaggccgcctggatcagttgctcaggcacgtgatgaaccccttggaaaccctctcaataactaactgccggctttcggaaggggatgtgatgcatctgtcccagagtcccagcgtcagtcagctaagtgtcctgagtctaagtggggtcatgctgaccgatgtaagtcccgagcccctccaagctctgctggagagagcctctgccaccctccaggacctggtctttgatgagtgtgggatcacggatgatcagctccttgccctcctgccttccctgagccactgctcccagcttacgaccttaagcttctacgggaattccatctccatatctgccctgcagagtctcctgcagcacctcatcgggctgagcaatctgacccacgtgctgtatcctgtccccctggagagttatgaggacatccatggtaccctccacctggagaggcttgcctatctgcatgccaggctcagggagttgctgtgtgagttggggcggcccagcatggtctggcttagtgccaacccctgtcctcactgtggggacagaaccttctatgacccggagcccatcctgtgcccctgtttcatgcctaattagctgggtgcacatatcaaatgcttcattctgcatacttggacactaaagccaggatgtgcatgcatcttgaagcaacaaagcagccacagtttcagacaaatgttcagtgtgagtgaggaaaacatgttcagtgaggaaaaaacattcagacaaatgttcagtgaggaaaaaaaggggaagttgggggtaggcagatgttgacttgaggagttaatgtgatctttggggagatacatcttatagagttagaaatagaatctgaatttctaaagggagattctggcttgggaagtacatgtaggagttaatccctgtgtagactgttgtaaagaaactgttgaaaataaagagaagcaatgtgaagca

SEQ ID NO: 27 - DNA, 2082 bp, Homo sapiens
  source 1..2082: mol_type = genomic DNA
acgcagcggctagggcgcttagctgagccattgtctcgttctttccttccagcaacttcgcggtgtggtgaactctctgaggaaaaaccattttgattattactctcagacgtgcgtggcaacaagtgactgagacctagaaatccaagcgttggaggtcctgaggccagcctaagtcgcttcaaaatggaacgaaggcgtttgtggggttccattcagagccgatacatcagcatgagtgtgtggacaagcccacggagacttgtggagctggcagggcagagcctgctgaaggatgaggccctggccattgccgccctggagttgctgcccagggagctcttcccgccactcttcatggcagcctttgacgggagacacagccagaccctgaaggcaatggtgcaggcctggcccttcacctgcctccctctgggagtgctgatgaagggacaacatcttcacctggagaccttcaaagctgtgcttgatggacttgatgtgctccttgcccaggaggttcgccccaggaggtggaaacttcaagtgctggatttacggaagaactctcatcaggacttctggactgtatggtctggaaacagggccagtctgtactcatttccagagccagaagcagctcagcccatgacaaagaagcgaaaagtagatggtttgagcacagaggcagagcagcccttcattccagtagaggtgctcgtagacctgttcctcaaggaaggtgcctgtgatgaattgttctcctacctcattgagaaagtgaagcgaaagaaaaatgtactacgcctgtgctgtaagaagctgaagatttttgcaatgcccatgcaggatatcaagatgatcctgaaaatggtgcagctggactctattgaagatttggaagtgacttgtacctggaagctacccaccttggcgaaattttctccttacctgggccagatgattaatctgcgtagactcctcctctcccacatccatgcatcttcctacatttccccggagaaggaagagcagtatatcgcccagttcacctctcagttcctcagtctgcagtgcctgcaggctctctatgtggactctttatttttccttagaggccgcctggatcagttgctcaggcacgtgatgaaccccttggaaaccctctcaataactaactgccggctttcggaaggggatgtgatgcatctgtcccagagtcccagcgtcagtcagctaagtgtcctgagtctaagtggggtcatgctgaccgatgtaagtcccgagcccctccaagctctgctggagagagcctctgccaccctccaggacctggtctttgatgagtgtgggatcacggatgatcagctccttgccctcctgccttccctgagccactgctcccagcttacgaccttaagcttctacgggaattccatctccatatctgccctgcagagtctcctgcagcacctcatcgggctgagcaatctgacccacgtgctgtatcctgtccccctggagagttatgaggacatccatggtaccctccacctggagaggcttgcctatctgcatgccaggctcagggagttgctgtgtgagttggggcggcccagcatggtctggcttagtgccaacccctgtcctcactgtggggacagaaccttctatgacccggagcccatcctgtgcccctgtttcatgcctaattagctgggtgcacatatcaaatgcttcattctgcatacttggacactaaagccaggatgtgcatgcatcttgaagcaacaaagcagccacagtttcagacaaatgttcagtgtgagtgaggaaaacatgttcagtgaggaaaaaacattcagacaaatgttcagtgaggaaaaaaaggggaagttgggggtaggcagatgttgacttgaggagttaatgtgatctttggggagatacatcttatagagttagaaatagaatctgaatttctaaagggagattctggcttgggaagtacatgtaggagttaatccctgtgtagactgttgtaaagaaactgttgaaaataaagagaagcaatgtgaagca

SEQ ID NO: 28 - DNA, 2061 bp, Homo sapiens
  source 1..2061: mol_type = genomic DNA
acgcagcggctagggcgcttagctgagccattgtctcgttctttccttccagcaacttcgcggtgtggtgaactctctgaggaaaaacacgtgcgtggcaacaagtgactgagacctagaaatccaagcgttggaggtcctgaggccagcctaagtcgcttcaaaatggaacgaaggcgtttgtggggttccattcagagccgatacatcagcatgagtgtgtggacaagcccacggagacttgtggagctggcagggcagagcctgctgaaggatgaggccctggccattgccgccctggagttgctgcccagggagctcttcccgccactcttcatggcagcctttgacgggagacacagccagaccctgaaggcaatggtgcaggcctggcccttcacctgcctccctctgggagtgctgatgaagggacaacatcttcacctggagaccttcaaagctgtgcttgatggacttgatgtgctccttgcccaggaggttcgccccaggaggtggaaacttcaagtgctggatttacggaagaactctcatcaggacttctggactgtatggtctggaaacagggccagtctgtactcatttccagagccagaagcagctcagcccatgacaaagaagcgaaaagtagatggtttgagcacagaggcagagcagcccttcattccagtagaggtgctcgtagacctgttcctcaaggaaggtgcctgtgatgaattgttctcctacctcattgagaaagtgaagcgaaagaaaaatgtactacgcctgtgctgtaagaagctgaagatttttgcaatgcccatgcaggatatcaagatgatcctgaaaatggtgcagctggactctattgaagatttggaagtgacttgtacctggaagctacccaccttggcgaaattttctccttacctgggccagatgattaatctgcgtagactcctcctctcccacatccatgcatcttcctacatttccccggagaaggaagagcagtatatcgcccagttcacctctcagttcctcagtctgcagtgcctgcaggctctctatgtggactctttatttttccttagaggccgcctggatcagttgctcaggcacgtgatgaaccccttggaaaccctctcaataactaactgccggctttcggaaggggatgtgatgcatctgtcccagagtcccagcgtcagtcagctaagtgtcctgagtctaagtggggtcatgctgaccgatgtaagtcccgagcccctccaagctctgctggagagagcctctgccaccctccaggacctggtctttgatgagtgtgggatcacggatgatcagctccttgccctcctgccttccctgagccactgctcccagcttacgaccttaagcttctacgggaattccatctccatatctgccctgcagagtctcctgcagcacctcatcgggctgagcaatctgacccacgtgctgtatcctgtccccctggagagttatgaggacatccatggtaccctccacctggagaggcttgcctatctgcatgccaggctcagggagttgctgtgtgagttggggcggcccagcatggtctggcttagtgccaacccctgtcctcactgtggggacagaaccttctatgacccggagcccatcctgtgcccctgtttcatgcctaattagctgggtgcacatatcaaatgcttcattctgcatacttggacactaaagccaggatgtgcatgcatcttgaagcaacaaagcagccacagtttcagacaaatgttcagtgtgagtgaggaaaacatgttcagtgaggaaaaaacattcagacaaatgttcagtgaggaaaaaaaggggaagttgggggtaggcagatgttgacttgaggagttaatgtgatctttggggagatacatcttatagagttagaaatagaatctgaatttctaaagggagattctggcttgggaagtacatgtaggagttaatccctgtgtagactgttgtaaagaaactgttgaaaataaagagaagcaatgtgaagca

SEQ ID NO: 29 - DNA, 2206 bp, Homo sapiens
  source 1..2206: mol_type = genomic DNA
acagctcccccgcagccagaagccgggcctgcagcgcctcagcaccgctccgggacaccccacccgcttcccaggcgtgacctgtcaacaggtctgtattggcgacaaaaggagcagccctgaatgtagggaaagcagggcggagtcctctgcaggctcgggggaggggaggggcgtgaatgcgtggatttctgtggagagtggaaacacggggagtcgaggggagcatgcgcgggcctcagaaagttctgggaaaccgactcccgggagcagggaggaacgcgcgctccagagacaacttcgcggtgtggtgaactctctgaggaaaaacggttccattcagagccgatacatcagcatgagtgtgtggacaagcccacggagacttgtggagctggcagggcagagcctgctgaaggatgaggccctggccattgccgccctggagttgctgcccagggagctcttcccgccactcttcatggcagcctttgacgggagacacagccagaccctgaaggcaatggtgcaggcctggcccttcacctgcctccctctgggagtgctgatgaagggacaacatcttcacctggagaccttcaaagctgtgcttgatggacttgatgtgctccttgcccaggaggttcgccccaggaggtggaaacttcaagtgctggatttacggaagaactctcatcaggacttctggactgtatggtctggaaacagggccagtctgtactcatttccagagccagaagcagctcagcccatgacaaagaagcgaaaagtagatggtttgagcacagaggcagagcagcccttcattccagtagaggtgctcgtagacctgttcctcaaggaaggtgcctgtgatgaattgttctcctacctcattgagaaagtgaagcgaaagaaaaatgtactacgcctgtgctgtaagaagctgaagatttttgcaatgcccatgcaggatatcaagatgatcctgaaaatggtgcagctggactctattgaagatttggaagtgacttgtacctggaagctacccaccttggcgaaattttctccttacctgggccagatgattaatctgcgtagactcctcctctcccacatccatgcatcttcctacatttccccggagaaggaagagcagtatatcgcccagttcacctctcagttcctcagtctgcagtgcctgcaggctctctatgtggactctttatttttccttagaggccgcctggatcagttgctcaggcacgtgatgaaccccttggaaaccctctcaataactaactgccggctttcggaaggggatgtgatgcatctgtcccagagtcccagcgtcagtcagctaagtgtcctgagtctaagtggggtcatgctgaccgatgtaagtcccgagcccctccaagctctgctggagagagcctctgccaccctccaggacctggtctttgatgagtgtgggatcacggatgatcagctccttgccctcctgccttccctgagccactgctcccagcttacgaccttaagcttctacgggaattccatctccatatctgccctgcagagtctcctgcagcacctcatcgggctgagcaatctgacccacgtgctgtatcctgtccccctggagagttatgaggacatccatggtaccctccacctggagaggcttgcctatctgcatgccaggctcagggagttgctgtgtgagttggggcggcccagcatggtctggcttagtgccaacccctgtcctcactgtggggacagaaccttctatgacccggagcccatcctgtgcccctgtttcatgcctaattagctgggtgcacatatcaaatgcttcattctgcatacttggacactaaagccaggatgtgcatgcatcttgaagcaacaaagcagccacagtttcagacaaatgttcagtgtgagtgaggaaaacatgttcagtgaggaaaaaacattcagacaaatgttcagtgaggaaaaaaaggggaagttgggggtaggcagatgttgacttgaggagttaatgtgatctttggggagatacatcttatagagttagaaatagaatctgaatttctaaagggagattctggcttgggaagtacatgtaggagttaatccctgtgtagactgttgtaaagaaactgttgaaaataaagagaagcaatgtgaagca

SEQ ID NO: 30 - DNA, 1963 bp, Homo sapiens
  source 1..1963: mol_type = genomic DNA
acgcagcggctagggcgcttagctgagccattgtctcgttctttccttccagcaacttcgcggtgtggtgaactctctgaggaaaaacggttccattcagagccgatacatcagcatgagtgtgtggacaagcccacggagacttgtggagctggcagggcagagcctgctgaaggatgaggccctggccattgccgccctggagttgctgcccagggagctcttcccgccactcttcatggcagcctttgacgggagacacagccagaccctgaaggcaatggtgcaggcctggcccttcacctgcctccctctgggagtgctgatgaagggacaacatcttcacctggagaccttcaaagctgtgcttgatggacttgatgtgctccttgcccaggaggttcgccccaggaggtggaaacttcaagtgctggatttacggaagaactctcatcaggacttctggactgtatggtctggaaacagggccagtctgtactcatttccagagccagaagcagctcagcccatgacaaagaagcgaaaagtagatggtttgagcacagaggcagagcagcccttcattccagtagaggtgctcgtagacctgttcctcaaggaaggtgcctgtgatgaattgttctcctacctcattgagaaagtgaagcgaaagaaaaatgtactacgcctgtgctgtaagaagctgaagatttttgcaatgcccatgcaggatatcaagatgatcctgaaaatggtgcagctggactctattgaagatttggaagtgacttgtacctggaagctacccaccttggcgaaattttctccttacctgggccagatgattaatctgcgtagactcctcctctcccacatccatgcatcttcctacatttccccggagaaggaagagcagtatatcgcccagttcacctctcagttcctcagtctgcagtgcctgcaggctctctatgtggactctttatttttccttagaggccgcctggatcagttgctcaggcacgtgatgaaccccttggaaaccctctcaataactaactgccggctttcggaaggggatgtgatgcatctgtcccagagtcccagcgtcagtcagctaagtgtcctgagtctaagtggggtcatgctgaccgatgtaagtcccgagcccctccaagctctgctggagagagcctctgccaccctccaggacctggtctttgatgagtgtgggatcacggatgatcagctccttgccctcctgccttccctgagccactgctcccagcttacgaccttaagcttctacgggaattccatctccatatctgccctgcagagtctcctgcagcacctcatcgggctgagcaatctgacccacgtgctgtatcctgtccccctggagagttatgaggacatccatggtaccctccacctggagaggcttgcctatctgcatgccaggctcagggagttgctgtgtgagttggggcggcccagcatggtctggcttagtgccaacccctgtcctcactgtggggacagaaccttctatgacccggagcccatcctgtgcccctgtttcatgcctaattagctgggtgcacatatcaaatgcttcattctgcatacttggacactaaagccaggatgtgcatgcatcttgaagcaacaaagcagccacagtttcagacaaatgttcagtgtgagtgaggaaaacatgttcagtgaggaaaaaacattcagacaaatgttcagtgaggaaaaaaaggggaagttgggggtaggcagatgttgacttgaggagttaatgtgatctttggggagatacatcttatagagttagaaatagaatctgaatttctaaagggagattctggcttgggaagtacatgtaggagttaatccctgtgtagactgttgtaaagaaactgttgaaaataaagagaagcaatgtgaagca

SEQ ID NO: 31 - DNA, 1966 bp, Homo sapiens
  source 1..1966: mol_type = genomic DNA
agacgcaaaagcccacacttccagtggtgtcagagagtatgagctccaggaggctttgatctcactgccccatgggcttgggccattagggggttccattcagagccgatacatcagcatgagtgtgtggacaagcccacggagacttgtggagctggcagggcagagcctgctgaaggatgaggccctggccattgccgccctggagttgctgcccagggagctcttcccgccactcttcatggcagcctttgacgggagacacagccagaccctgaaggcaatggtgcaggcctggcccttcacctgcctccctctgggagtgctgatgaagggacaacatcttcacctggagaccttcaaagctgtgcttgatggacttgatgtgctccttgcccaggaggttcgccccaggaggtggaaacttcaagtgctggatttacggaagaactctcatcaggacttctggactgtatggtctggaaacagggccagtctgtactcatttccagagccagaagcagctcagcccatgacaaagaagcgaaaagtagatggtttgagcacagaggcagagcagcccttcattccagtagaggtgctcgtagacctgttcctcaaggaaggtgcctgtgatgaattgttctcctacctcattgagaaagtgaagcgaaagaaaaatgtactacgcctgtgctgtaagaagctgaagatttttgcaatgcccatgcaggatatcaagatgatcctgaaaatggtgcagctggactctattgaagatttggaagtgacttgtacctggaagctacccaccttggcgaaattttctccttacctgggccagatgattaatctgcgtagactcctcctctcccacatccatgcatcttcctacatttccccggagaaggaagagcagtatatcgcccagttcacctctcagttcctcagtctgcagtgcctgcaggctctctatgtggactctttatttttccttagaggccgcctggatcagttgctcaggcacgtgatgaaccccttggaaaccctctcaataactaactgccggctttcggaaggggatgtgatgcatctgtcccagagtcccagcgtcagtcagctaagtgtcctgagtctaagtggggtcatgctgaccgatgtaagtcccgagcccctccaagctctgctggagagagcctctgccaccctccaggacctggtctttgatgagtgtgggatcacggatgatcagctccttgccctcctgccttccctgagccactgctcccagcttacgaccttaagcttctacgggaattccatctccatatctgccctgcagagtctcctgcagcacctcatcgggctgagcaatctgacccacgtgctgtatcctgtccccctggagagttatgaggacatccatggtaccctccacctggagaggcttgcctatctgcatgccaggctcagggagttgctgtgtgagttggggcggcccagcatggtctggcttagtgccaacccctgtcctcactgtggggacagaaccttctatgacccggagcccatcctgtgcccctgtttcatgcctaattagctgggtgcacatatcaaatgcttcattctgcatacttggacactaaagccaggatgtgcatgcatcttgaagcaacaaagcagccacagtttcagacaaatgttcagtgtgagtgaggaaaacatgttcagtgaggaaaaaacattcagacaaatgttcagtgaggaaaaaaaggggaagttgggggtaggcagatgttgacttgaggagttaatgtgatctttggggagatacatcttatagagttagaaatagaatctgaatttctaaagggagattctggcttgggaagtacatgtaggagttaatccctgtgtagactgttgtaaagaaactgttgaaaataaagagaagcaatgtgaagca

SEQ ID NO: 32 - DNA, 2228 bp, Homo sapiens
  source 1..2228: mol_type = genomic DNA
aaaagcaccagtgggtgatcaggcccaggataggaatcatccctaagcagatccttaaatgccaaatataacttcaattttttgtgacggaaaatgtatttgtgcactgtgcctagcatggaaatagagaaaaactgagtctaattgataggcctgattgcatctcgctgaccagaatctctgtacctgagcagcttaggggcaagaaagaacagctgttttcttgaagtgtggaaacattagtaatacttaggaaggggctggccattggagattatgtttgtggtaaaggcctgacctgctgcttgtgaacagccccagtgtggaagaggacacctaaccataaagagaagggttccattcagagccgatacatcagcatgagtgtgtggacaagcccacggagacttgtggagctggcagggcagagcctgctgaaggatgaggccctggccattgccgccctggagttgctgcccagggagctcttcccgccactcttcatggcagcctttgacgggagacacagccagaccctgaaggcaatggtgcaggcctggcccttcacctgcctccctctgggagtgctgatgaagggacaacatcttcacctggagaccttcaaagctgtgcttgatggacttgatgtgctccttgcccaggaggttcgccccaggaggtggaaacttcaagtgctggatttacggaagaactctcatcaggacttctggactgtatggtctggaaacagggccagtctgtactcatttccagagccagaagcagctcagcccatgacaaagaagcgaaaagtagatggtttgagcacagaggcagagcagcccttcattccagtagaggtgctcgtagacctgttcctcaaggaaggtgcctgtgatgaattgttctcctacctcattgagaaagtgaagcgaaagaaaaatgtactacgcctgtgctgtaagaagctgaagatttttgcaatgcccatgcaggatatcaagatgatcctgaaaatggtgcagctggactctattgaagatttggaagtgacttgtacctggaagctacccaccttggcgaaattttctccttacctgggccagatgattaatctgcgtagactcctcctctcccacatccatgcatcttcctacatttccccggagaaggaagagcagtatatcgcccagttcacctctcagttcctcagtctgcagtgcctgcaggctctctatgtggactctttatttttccttagaggccgcctggatcagttgctcaggcacgtgatgaaccccttggaaaccctctcaataactaactgccggctttcggaaggggatgtgatgcatctgtcccagagtcccagcgtcagtcagctaagtgtcctgagtctaagtggggtcatgctgaccgatgtaagtcccgagcccctccaagctctgctggagagagcctctgccaccctccaggacctggtctttgatgagtgtgggatcacggatgatcagctccttgccctcctgccttccctgagccactgctcccagcttacgaccttaagcttctacgggaattccatctccatatctgccctgcagagtctcctgcagcacctcatcgggctgagcaatctgacccacgtgctgtatcctgtccccctggagagttatgaggacatccatggtaccctccacctggagaggcttgcctatctgcatgccaggctcagggagttgctgtgtgagttggggcggcccagcatggtctggcttagtgccaacccctgtcctcactgtggggacagaaccttctatgacccggagcccatcctgtgcccctgtttcatgcctaattagctgggtgcacatatcaaatgcttcattctgcatacttggacactaaagccaggatgtgcatgcatcttgaagcaacaaagcagccacagtttcagacaaatgttcagtgtgagtgaggaaaacatgttcagtgaggaaaaaacattcagacaaatgttcagtgaggaaaaaaaggggaagttgggggtaggcagatgttgacttgaggagttaatgtgatctttggggagatacatcttatagagttagaaatagaatctgaatttctaaagggagattctggcttgggaagtacatgtaggagttaatccctgtgtagactgttgtaaagaaactgttgaaaataaagagaagcaatgtgaagca

SEQ ID NO: 33 - protein, 509 aa, Homo sapiens
MERRRLWGSIQSRYISMSVWTSPRRLVELAGQSLLKDEALAIAALELLPRELFPPLFMAAFDGRHSQTLKAMVQAWPFTCLPLGVLMKGQHLHLETFKAVLDGLDVLLAQEVRPRRWKLQVLDLRKNSHQDFWTVWSGNRASLYSFPEPEAAQPMTKKRKVDGLSTEAEQPFIPVEVLVDLFLKEGACDELFSYLIEKVKRKKNVLRLCCKKLKIFAMPMQDIKMILKMVQLDSIEDLEVTCTWKLPTLAKFSPYLGQMINLRRLLLSHIHASSYISPEKEEQYIAQFTSQFLSLQCLQALYVDSLFFLRGRLDQLLRHVMNPLETLSITNCRLSEGDVMHLSQSPSVSQLSVLSLSGVMLTDVSPEPLQALLERASATLQDLVFDECGITDDQLLALLPSLSHCSQLTTLSFYGNSISISALQSLLQHLIGLSNLTHVLYPVPLESYEDIHGTLHLERLAYLHARLRELLCELGRPSMVWLSANPCPHCGDRTFYDPEPILCPCFMPN

SEQ ID NO: 34 - protein, 493 aa, Homo sapiens
MSVWTSPRRLVELAGQSLLKDEALAIAALELLPRELFPPLFMAAFDGRHSQTLKAMVQAWPFTCLPLGVLMKGQHLHLETFKAVLDGLDVLLAQEVRPRRWKLQVLDLRKNSHQDFWTVWSGNRASLYSFPEPEAAQPMTKKRKVDGLSTEAEQPFIPVEVLVDLFLKEGACDELFSYLIEKVKRKKNVLRLCCKKLKIFAMPMQDIKMILKMVQLDSIEDLEVTCTWKLPTLAKFSPYLGQMINLRRLLLSHIHASSYISPEKEEQYIAQFTSQFLSLQCLQALYVDSLFFLRGRLDQLLRHVMNPLETLSITNCRLSEGDVMHLSQSPSVSQLSVLSLSGVMLTDVSPEPLQALLERASATLQDLVFDECGITDDQLLALLPSLSHCSQLTTLSFYGNSISISALQSLLQHLIGLSNLTHVLYPVPLESYEDIHGTLHLERLAYLHARLRELLCELGRPSMVWLSANPCPHCGDRTFYDPEPILCPCFMPN

SEQ ID NO: 35 - DNA, 1098 bp, Homo sapiens
  source 1..1098: mol_type = genomic DNA
atggccgtcatggcgccccgaaccctcgtcctgctactctcgggggctctggccctgacccagacctgggcgggctctcactccatgaggtatttcttcacatccgtgtcccggcccggccgcggggagccccgcttcatcgcagtgggctacgtggacgacacgcagttcgtgcggttcgacagcgacgccgcgagccagaggatggagccgcgggcgccgtggatagagcaggagggtccggagtattgggacggggagacacggaaagtgaaggcccactcacagactcaccgagtggacctggggaccctgcgcggctactacaaccagagcgaggccggttctcacaccgtccagaggatgtatggctgcgacgtggggtcggactggcgcttcctccgcgggtaccaccagtacgcctacgacggcaaggattacatcgccctgaaagaggacctgcgctcttggaccgcggcggacatggcagctcagaccaccaagcacaagtgggaggcggcccatgtggcggagcagttgagagcctacctggagggcacgtgcgtggagtggctccgcagatacctggagaacgggaaggagacgctgcagcgcacggacgcccccaaaacgcatatgactcaccacgctgtctctgaccatgaagccaccctgaggtgctgggccctgagcttctaccctgcggagatcacactgacctggcagcgggatggggaggaccagacccaggacacggagctcgtggagaccaggcctgcaggggatggaaccttccagaagtgggcggctgtggtggtgccttctggacaggagcagagatacacctgccatgtgcagcatgagggtttgcccaagcccctcaccctgagatgggagccgtcttcccagcccaccatccccatcgtgggcatcattgctggcctggttctctttggagctgtgatcactggagctgtggtcgctgctgtgatgtggaggaggaagagctcagatagaaaaggagggagctactctcaggctgcaagcagtgacagtgcccagggctctgatgtgtctctcacagcttgtaaagtgtga

SEQ ID NO: 36 - protein, 365 aa, Homo sapiens [sequence omitted]

SEQ ID NO: 37 - DNA, 9683 bp, synthetic construct
  source 1..9683: mol_type = other DNA
tggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgccgaattaattcacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacaggcgcgccagagatccagtttggacctgcaggtgaaagaccccacctgtaggtttggcaagttagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagcgaaccatcagatgtttccagggtgccccaaggacctgaaatgaccctgtgccttatttgaactaaccaatcagtttgcttcttgcttctgtttgtgtgcttctgctccctgagctcaataaaagagcccacaacccctcacttggtgggccagtcctctgatagactgtgtcccctggatacccgtacggtaccgctagcgccaccatgggcaccagcctcctctgctggatggccctgtgtctcctgggggcagatcacgcagatactggagtctcccaggaccccagacacaagatcacaaagaggggacagaatgttactttcaggtgtgatccaatttctgaacacaaccgcctttattggtaccgccagaccctggggcagggcccagagtttctgacttacttccagaatgaagctcaacttgaaaaatcaaggctgctcagtgatcggttctctgcagagaggcctaagggatctttctccaccttggagatccagcgcacagagcagggggactctgccatgtatctctgtgccagcagccgcactgctggagatactcagtattttggcccaggcacccggctgacagtgctcgaagatctgaacaaggtgttccctccagaggtggccgtgttcgagccttctaaggccgagatcgcccacacacaaaaagccaccctcgtgtgcctggccaccggctttttccccgaccacgtggaactgtcttggtgggtcaacggcaaagaggtgcactccggcgtgtcaacggatccccagcctctgaaagaacagcctgccctgaacgacagccggtactgcctgagctccagactgagagtgtccgccaccttctggcagaacccccggaaccacttcagatgccaggtgcagttttacggcctgagcgagaacgacgagtggacccaggacagagccaagcccgtgacacaaatcgtgtctgccgaagcctggggaagagccgattgcggcatcaccagcgcctcctatcaccagggcgtgctgagcgccacaatcctgtacgaaatcctgctgggcaaggccaccctgtacgccgtgctggtgtctgctctggtgctgatggccatggtcaagcggaaggactttggcagcggcagagccaaaaggtccgggagcggtgcgacaaactttagcctgttgaaacaagccggcgacgttgaagagaaccccggacctatggaaaccctcttgggcctgcttatcctttggctgcagctgcaatgggtgagcagcaaacaggaggtgactcagattcctgcagctctgagtgtcccagaaggagaaaacttggttctcaactgcagtttcactgatagcgctatttacaacctccagtggtttaggcaggaccctgggaaaggcctcacatctctgttgcttattcagtcaagtcagagagagcaaacaagtggacgccttaatgcctctctggataaatcatcaggacgcagtactctttacattgcagcttctcagcctggtgattcagccacctacctgtgcgctgtgaggggtggtacctcaggaacctacaaatacatctttggaacaggcaccaggctgaaggttcttgcaaacatccagaaccccgaccccgccgtgtaccagctgagggactccaagtccagcgacaagagcgtgtgtctgtttacggacttcgacagccagaccaacgtgagtcaaagcaaggacagcgacgtctacataacggataagaccgtgctggacatgcggagcatggacttcaagagcaacagcgccgtggcctggtccaacaagagcgacttcgcctgcgccaacgccttcaacaacagcatcatccccgaggacaccttcttccccagcagcgacgtgccctgcgacgtgaaactggtggagaagtccttcgagacagacaccaatctgaactttcagaacctgctggtgatcgtgctgcggattctgctgctgaaagtggccggcttcaatctgctgatgaccctgcggctgtggagcagcagggctaagaggtccggcagcggagccaccaatttttccctgctgaaacaggctggtgacgtggaagaaaaccctggccccatggcgctgcccgtcaccgcgctgctgctgcccctggcgctgctgttacacgccgctcggccagagcttcccacccagggcacattctccaacgtgtccaccaatgtgtcgggaggcggcggatcgtcccagttcagagtgtcccctctggaccgcacctggaacctgggcgagaccgtggagctgaaatgtcaggtcctgctgagcaacccgacctccgggtgcagttggctgttccagccgcgtggtgctgccgcaagccctacgttcctgctttacctgagccagaacaagcccaaggcggccgagggcctggacacccagagattctccggcaagcgcctgggggacacattcgtgcttactttgagcgatttccgcagagagaacgagggctactatttctgttcggcgctgagcaattccatcatgtatttcagccactttgtgccagtgttcctgcctgccaagcctaccacaacaccagctccccgtcccccgactccggcgcctaccatcgcgagtcaaccgttgagcctgaggcctgaggcttgtcggcccgctgcggggggtgccgtccacaccaggggcctcgactttgcgtgcgacatctatatttgggcgcctctggcgggtacctgcggggtgctgctgctgtcattggtgattaccctgtactgcaatcaccgcaaccgccggcgggtctgtaagtgcccacggcctgtggtcaagtccggtgacaaaccgtcgctctcggctcgctacgtgcgcgctaagcgcagcggttccggggccaccaacttttcattgctgaagcaggccggtgatgtggaggagaatccagggcccatgcgccccaggctttggctccttcttgctgctcagctcactgtcttgcatggcaactccgttctgcagcagactcccgcctacatcaaggtgcagacgaacaagatggtgatgctgtcatgcgaggccaagatctctctttcaaatatgagaatttattggctacgacagcgccaggccccctccagcgacagccaccacgagttcctggcgctttgggattctgctaaaggcaccatccatggagaggaggtggaacaggagaagatagctgtcttccgcgacgcatcccgcttcatcctgaacctgaccagcgtgaagccggaggacagcggcatctacttctgtatgatcgttggctcccccgagctgaccttcggcaaaggcacccagctgtccgtggtggacttcctgcccaccacagcccagccaaccaagaaatccaccctcaagaagcgcgtgtgccgactgccccgccctgaaacccagaagggccctctgtgctcccccatcacccttggactgctggtggcgggagtcctggtgctgctcgtatctctgggtgtcgccatccacctgtgctgccgccgccgccgcgcccgcctgaggtttatgaaacagttttacaagtgataaatcgatagatcctaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgagatcctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggcccgggttaattaaggaaagggctagatcattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagcaagctcatggctgactaattttttttatttatgcagaggccgaggccgcctcggcctctgagctattccagaagtagtgaggaggcttttttggaggcctaggcttttgcaaaaagctccccgtggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgacatgattacgaatttcacaaataaagcatttttttcactgcattctagttgtggtttgtccaaactcatcaatgtatcttatcatgtctggatcaactggataactcaagctaaccaaaatcatcccaaacttcccaccccataccctattaccactgccaattacctgtggtttcatttactctaaacctgtgattcctctgaattattttcattttaaagaaattgtatttgttaaatatgtactacaaacttagtagt

SEQ ID NO: 38 - DNA, 8789 bp, synthetic construct
  source 1..8789: mol_type = other DNA
tggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgccgaattaattcacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacaggcgcgccagagatccagtttggacctgcaggtgaaagaccccacctgtaggtttggcaagttagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagcgaaccatcagatgtttccagggtgccccaaggacctgaaatgaccctgtgccttatttgaactaaccaatcagtttgcttcttgcttctgtttgtgtgcttctgctccctgagctcaataaaagagcccacaacccctcacttggtgggccagtcctctgatagactgtgtcccctggatacccgtacggtaccgctagcgccaccatgggctcctggaccctctgctgtgtgtccctttgcatcctggttgcaaagcacacagatgctggagttatccagtcaccccggcacgaggtgacagagatgggacaagaagtgactctgagatgtaaaccaatttcaggacatgactaccttttctggtacagacagaccatgatgcggggactggagttgctcatttactttaacaacaacgttcctattgatgattcagggatgcccgaggatcgcttctcagctaagatgcctaatgcatcattctccactctgaagatccagccctcagaacccagggactcagctgtgtacttctgtgccagcagttttctcggctggaatgaaaaactgttctttggcagtggaacccagctctctgtcttggaagatctgaacaaggtgttccctccagaggtggccgtgttcgagccttctaaggccgagatcgcccacacacaaaaagccaccctcgtgtgcctggccaccggctttttccccgaccacgtggaactgtcttggtgggtcaacggcaaagaggtgcactccggcgtgtcaacggatccccagcctctgaaagaacagcctgccctgaacgacagccggtactgcctgagctccagactgagagtgtccgccaccttctggcagaacccccggaaccacttcagatgccaggtgcagttttacggcctgagcgagaacgacgagtggacccaggacagagccaagcccgtgacacaaatcgtgtctgccgaagcctggggaagagccgattgcggcatcaccagcgcctcctatcaccagggcgtgctgagcgccacaatcctgtacgaaatcctgctgggcaaggccaccctgtacgccgtgctggtgtctgctctggtgctgatggccatggtcaagcggaaggactttggcagcggcagagccaaaaggtccgggagcggtgcgacaaactttagcctgttgaaacaagccggcgacgttgaagagaaccccggacctatggtcctgaaattctccgtgtccattctttggattcagttggcatgggtgagcacccagctgctggagcagagccctcagtttcttagcatccaagagggagaaaatctcactgtgtactgcaactcctcaagtgttttctccagccttcaatggtacagacaggagcctggggaaggtcctgtcctcctggtgacagttgttactggtggagaagtgaagaagctgaagagacttacctttcagtttggtgatgcaagaaaggacagttctctccacatcactgcagcccagcctggtgatacaggcctctacctctgtgcaggagatgaaagtattagctatggaaagctgacatttggacaagggaccatcttgactgtccatccaaacatccagaaccccgaccccgccgtgtaccagctgagggactccaagtccagcgacaagagcgtgtgtctgtttacggacttcgacagccagaccaacgtgagtcaaagcaaggacagcgacgtctacataacggataagaccgtgctggacatgcggagcatggacttcaagagcaacagcgccgtggcctggtccaacaagagcgacttcgcctgcgccaacgccttcaacaacagcatcatccccgaggacaccttcttccccagcagcgacgtgccctgcgacgtgaaactggtggagaagtccttcgagacagacaccaatctgaactttcagaacctgctggtgatcgtgctgcggattctgctgctgaaagtggccggcttcaatctgctgatgaccctgcggctgtggagcagcagggctaagaggtccggcagcggagccaccaatttttccctgctgaaacaggctggtgacgtggaagaaaaccctggccccatgggtcgggggctgctcaggggcctgtggccgctgcacatcgtcctgtggacgcgtatcgccagcacgatcccaccgcacgttcagaagtcggttaataacgacatgatagtcactgacaacaacggtgcagtcaagtttccacaactgtgtaaattttgtgatgtgagattttccacctgtgacaaccagaaatcctgcatgagcaactgcagcatcacctccatctgtgagaagccacaggaagtctgtgtggctgtatggagaaagaatgacgagaacataacactagagacagtttgccatgaccccaagctcccctaccatgactttattctggaagatgctgcttctccaaagtgcattatgaaggaaaaaaaaaagcctggtgagactttcttcatgtgttcctgtagctctgatgagtgcaatgacaacatcatcttctcagaagaatataacaccagcaatcctgacttgttgctagtcatatttcaagtgacaggcatcagcctcctgccaccactgggagttgccatatctgtcatcatcatcttctactgctaccgcgttaaccggcagcagaagtagtgataaatcgatagatcctaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgagatcctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggcccgggttaattaaggaaagggctagatcattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagcaagctcatggctgactaattttttttatttatgcagaggccgaggccgcctcggcctctgagctattccagaagtagtgaggaggcttttttggaggcctaggcttttgcaaaaagctccccgtggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgacatgattacgaatttcacaaataaagcatttttttcactgcattctagttgtggtttgtccaaactcatcaatgtatcttatcatgtctggatcaactggataactcaagctaaccaaaatcatcccaaacttcccaccccataccctattaccactgccaattacctgtggtttcatttactctaaacctgtgattcctctgaattattttcattttaaagaaattgtatttgttaaatatgtactacaaacttagtagt

SEQ ID NO: 39 - DNA, 816 bp, synthetic construct
  source 1..816: mol_type = other DNA
atggagaagaatcctttggcagccccattactaatcctctggtttcatcttgactgcgtgagcagcatactgaacgtggaacaaagtcctcagtcactgcatgttcaggagggagacagcaccaatttcacctgcagcttcccttccagcaatttttatgccttacactggtacagatgggaaactgcaaaaagccccgaggccttgtttgtaatgactttaaatggggatgaaaagaagaaaggacgaataagtgccactcttaataccaaggagggttacagctatttgtacatcaaaggatcccagcctgaagactcagccacatacctctgtgccctgtacaataacaatgacatgcgctttggagcagggaccagactgacagtaaaaccaaacatccagaaccccgaccccgccgtgtaccagctgagggactccaagtccagcgacaagagcgtgtgtctgtttacggacttcgacagccagaccaacgtgagtcaaagcaaggacagcgacgtctacataacggataagaccgtgctggacatgcggagcatggacttcaagagcaacagcgccgtggcctggtccaacaagagcgacttcgcctgcgccaacgccttcaacaacagcatcatccccgaggacaccttcttccccagcagcgacgtgccctgcgacgtgaaactggtggagaagtccttcgagacagacaccaatctgaactttcagaacctgctggtgatcgtgctgcggattctgctgctgaaagtggccggcttcaatctgctgatgaccctgcggctgtggagc

SEQ ID NO: 40 - protein, 272 aa, synthetic construct
MEKNPLAAPLLILWFHLDCVSSILNVEQSPQSLHVQEGDSTNFTCSFPSSNFYALHWYRWETAKSPEALFVMTLNGDEKKKGRISATLNTKEGYSYLYIKGSQPEDSATYLCALYNNNDMRFGAGTRLTVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSSDVPCDVKLVEKSFETDTNLNFQNLLVIVLRILLLKVAGFNLLMTLRLWS

SEQ ID NO: 41 - DNA, 960 bp, synthetic construct
  source 1..960: mol_type = other DNA
atggactcctggaccttctgctgtgtgtccctttgcatcctggtagcgaagcatacagatgctggagttatccagtcaccccgccatgaggtgacagagatgggacaagaagtgactctgagatgtaaaccaatttcaggccacaactcccttttctggtacagacagaccatgatgcggggactggagttgctcatttactttaacaacaacgttccgatagatgattcagggatgcccgaggatcgattctcagctaagatgcctaatgcatcattctccactctgaagatccagccctcagaacccagggactcagctgtgtacttctgtgccagcagtcccggcagcacagatacgcagtattttggcccaggcacccggctgacagtgctcgaagatctgaacaaggtgttccctccagaggtggccgtgttcgagccttctaaggccgagatcgcccacacacaaaaagccaccctcgtgtgcctggccaccggctttttccccgaccacgtggaactgtcttggtgggtcaacggcaaagaggtgcactccggcgtgtcaacggatccccagcctctgaaagaacagcctgccctgaacgacagccggtactgcctgagctccagactgagagtgtccgccaccttctggcagaacccccggaaccacttcagatgccaggtgcagttttacggcctgagcgagaacgacgagtggacccaggacagagccaagcccgtgacacaaatcgtgtctgccgaagcctggggaagagccgattgcggcatcaccagcgcctcctatcaccagggcgtgctgagcgccacaatcctgtacgaaatcctgctgggcaaggccaccctgtacgccgtgctggtgtctgctctggtgctgatggccatggtcaagcggaaggactttggcagcggcagagccaaaaggtccgggagcggt

SEQ ID NO: 42 - protein, 320 aa, synthetic construct
MDSWTFCCVSLCILVAKHTDAGVIQSPRHEVTEMGQEVTLRCKPISGHNSLFWYRQTMMRGLELLIYFNNNVPIDDSGMPEDRFSAKMPNASFSTLKIQPSEPRDSAVYFCASSPGSTDTQYFGPGTRLTVLEDLNKVFPPEVAVFEPSKAEIAHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGITSASYHQGVLSATILYEILLGKATLYAVLVSALVLMAMVKRKDFGSGRAKRSGSG

SEQ ID NO: 43 - DNA, 1833 bp, synthetic construct
  source 1..1833: mol_type = other DNA
atggactcctggaccttctgctgtgtgtccctttgcatcctggtagcgaagcatacagatgctggagttatccagtcaccccgccatgaggtgacagagatgggacaagaagtgactctgagatgtaaaccaatttcaggccacaactcccttttctggtacagacagaccatgatgcggggactggagttgctcatttactttaacaacaacgttccgatagatgattcagggatgcccgaggatcgattctcagctaagatgcctaatgcatcattctccactctgaagatccagccctcagaacccagggactcagctgtgtacttctgtgccagcagtcccggcagcacagatacgcagtattttggcccaggcacccggctgacagtgctcgaagatctgaacaaggtgttccctccagaggtggccgtgttcgagccttctaaggccgagatcgcccacacacaaaaagccaccctcgtgtgcctggccaccggctttttccccgaccacgtggaactgtcttggtgggtcaacggcaaagaggtgcactccggcgtgtcaacggatccccagcctctgaaagaacagcctgccctgaacgacagccggtactgcctgagctccagactgagagtgtccgccaccttctggcagaacccccggaaccacttcagatgccaggtgcagttttacggcctgagcgagaacgacgagtggacccaggacagagccaagcccgtgacacaaatcgtgtctgccgaagcctggggaagagccgattgcggcatcaccagcgcctcctatcaccagggcgtgctgagcgccacaatcctgtacgaaatcctgctgggcaaggccaccctgtacgccgtgctggtgtctgctctggtgctgatggccatggtcaagcggaaggactttggcagcggcagagccaaaaggtccgggagcggtgcgacaaactttagcctgttgaaacaagccggcgacgttgaagagaaccccggacctatggagaagaatcctttggcagccccattactaatcctctggtttcatcttgactgcgtgagcagcatactgaacgtggaacaaagtcctcagtcactgcatgttcaggagggagacagcaccaatttcacctgcagcttcccttccagcaatttttatgccttacactggtacagatgggaaactgcaaaaagccccgaggccttgtttgtaatgactttaaatggggatgaaaagaagaaaggacgaataagtgccactcttaataccaaggagggttacagctatttgtacatcaaaggatcccagcctgaagactcagccacatacctctgtgccctgtacaataacaatgacatgcgctttggagcagggaccagactgacagtaaaaccaaacatccagaaccccgaccccgccgtgtaccagctgagggactccaagtccagcgacaagagcgtgtgtctgtttacggacttcgacagccagaccaacgtgagtcaaagcaaggacagcgacgtctacataacggataagaccgtgctggacatgcggagcatggacttcaagagcaacagcgccgtggcctggtccaacaagagcgacttcgcctgcgccaacgccttcaacaacagcatcatccccgaggacaccttcttccccagcagcgacgtgccctgcgacgtgaaactggtggagaagtccttcgagacagacaccaatctgaactttcagaacctgctggtgatcgtgctgcggattctgctgctgaaagtggccggcttcaatctgctgatgaccctgcggctgtggagc

SEQ ID NO: 44 - protein, 611 aa, synthetic construct
MDSWTFCCVSLCILVAKHTDAGVIQSPRHEVTEMGQEVTLRCKPISGHNSLFWYRQTMMRGLELLIYFNNNVPIDDSGMPEDRFSAKMPNASFSTLKIQPSEPRDSAVYFCASSPGSTDTQYFGPGTRLTVLEDLNKVFPPEVAVFEPSKAEIAHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGITSASYHQGVLSATILYEILLGKATLYAVLVSALVLMAMVKRKDFGSGRAKRSGSGATNFSLLKQAGDVEENPGPMEKNPLAAPLLILWFHLDCVSSILNVEQSPQSLHVQEGDSTNFTCSFPSSNFYALHWYRWETAKSPEALFVMTLNGDEKKKGRISATLNTKEGYSYLYIKGSQPEDSATYLCALYNNNDMRFGAGTRLTVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSSDVPCDVKLVEKSFETDTNLNFQNLLVIVLRILLLKVAGFNLLMTLRLWS

SEQ ID NO: 45 - DNA, 816 bp, synthetic construct
  source 1..816: mol_type = other DNA
atggagaagaatcctttggcagccccattactaatcctctggtttcatcttgactgcgtgagcagcatactgaacgtggaacaaagtcctcagtcactgcatgttcaggagggagacagcaccaatttcacctgcagcttcccttccagcaatttttatgccttacactggtacagaaaggaaactgcaaaaagccccgaggccttgtttgtaatgactttaaatggggatgaaaagaagaaaggacgaataagtgccactcttaataccaaggagggttacagctatttgtacatcaaaggatcccagcctgaagactcagccacatacctctgtgccctgtacaataacaatgacatgcgctttggagcagggaccagactgacagtaaaaccaaacatccagaaccccgaccccgccgtgtaccagctgagggactccaagtccagcgacaagagcgtgtgtctgtttacggacttcgacagccagaccaacgtgagtcaaagcaaggacagcgacgtctacataacggataagaccgtgctggacatgcggagcatggacttcaagagcaacagcgccgtggcctggtccaacaagagcgacttcgcctgcgccaacgccttcaacaacagcatcatccccgaggacaccttcttccccagcagcgacgtgccctgcgacgtgaaactggtggagaagtccttcgagacagacaccaatctgaactttcagaacctgctggtgatcgtgctgcggattctgctgctgaaagtggccggcttcaatctgctgatgaccctgcggctgtggagc

SEQ ID NO: 46 - protein, 272 aa, synthetic construct
MEKNPLAAPLLILWFHLDCVSSILNVEQSPQSLHVQEGDSTNFTCSFPSSNFYALHWYRKETAKSPEALFVMTLNGDEKKKGRISATLNTKEGYSYLYIKGSQPEDSATYLCALYNNNDMRFGAGTRLTVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSSDVPCDVKLVEKSFETDTNLNFQNLLVIVLRILLLKVAGFNLLMTLRLWS

SEQ ID NO: 47 - DNA, 960 bp, synthetic construct
  source 1..960: mol_type = other DNA
atggactcctggaccttctgctgtgtgtccctttgcatcctggtagcgaagcatacagatgctggagttatccagtcaccccgccatgaggtgacagagatgggacaagaagtgactctgagatgtaaaccaatttcaggccacaactcccttttctggtacagagagaccatgatgcggggactggagttgctcatttactttaacaacaacgttccgatagatgattcagggatgcccgaggatcgattctcagctaagatgcctaatgcatcattctccactctgaagatccagccctcagaacccagggactcagctgtgtacttctgtgccagcagtcccggcagcacagatacgcagtattttggcccaggcacccggctgacagtgctcgaagatctgaacaaggtgttccctccagaggtggccgtgttcgagccttctaaggccgagatcgcccacacacaaaaagccaccctcgtgtgcctggccaccggctttttccccgaccacgtggaactgtcttggtgggtcaacggcaaagaggtgcactccggcgtgtcaacggatccccagcctctgaaagaacagcctgccctgaacgacagccggtactgcctgagctccagactgagagtgtccgccaccttctggcagaacccccggaaccacttcagatgccaggtgcagttttacggcctgagcgagaacgacgagtggacccaggacagagccaagcccgtgacacaaatcgtgtctgccgaagcctggggaagagccgattgcggcatcaccagcgcctcctatcaccagggcgtgctgagcgccacaatcctgtacgaaatcctgctgggcaaggccaccctgtacgccgtgctggtgtctgctctggtgctgatggccatggtcaagcggaaggactttggcagcggcagagccaaaaggtccgggagcggt

SEQ ID NO: 48 - protein, 320 aa, synthetic construct
MDSWTFCCVSLCILVAKHTDAGVIQSPRHEVTEMGQEVTLRCKPISGHNSLFWYRETMMRGLELLIYFNNNVPIDDSGMPEDRFSAKMPNASFSTLKIQPSEPRDSAVYFCASSPGSTDTQYFGPGTRLTVLEDLNKVFPPEVAVFEPSKAEIAHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGITSASYHQGVLSATILYEILLGKATLYAVLVSALVLMAMVKRKDFGSGRAKRSGSG

SEQ ID NO: 49 - DNA, 1833 bp, synthetic construct
  source 1..1833: mol_type = other DNA
atggactcctggaccttctgctgtgtgtccctttgcatcctggtagcgaagcatacagatgctggagttatccagtcaccccgccatgaggtgacagagatgggacaagaagtgactctgagatgtaaaccaatttcaggccacaactcccttttctggtacagagagaccatgatgcggggactggagttgctcatttactttaacaacaacgttccgatagatgattcagggatgcccgaggatcgattctcagctaagatgcctaatgcatcattctccactctgaagatccagccctcagaacccagggactcagctgtgtacttctgtgccagcagtcccggcagcacagatacgcagtattttggcccaggcacccggctgacagtgctcgaagatctgaacaaggtgttccctccagaggtggccgtgttcgagccttctaaggccgagatcgcccacacacaaaaagccaccctcgtgtgcctggccaccggctttttccccgaccacgtggaactgtcttggtgggtcaacggcaaagaggtgcactccggcgtgtcaacggatccccagcctctgaaagaacagcctgccctgaacgacagccggtactgcctgagctccagactgagagtgtccgccaccttctggcagaacccccggaaccacttcagatgccaggtgcagttttacggcctgagcgagaacgacgagtggacccaggacagagccaagcccgtgacacaaatcgtgtctgccgaagcctggggaagagccgattgcggcatcaccagcgcctcctatcaccagggcgtgctgagcgccacaatcctgtacgaaatcctgctgggcaaggccaccctgtacgccgtgctggtgtctgctctggtgctgatggccatggtcaagcggaaggactttggcagcggcagagccaaaaggtccgggagcggtgcgacaaactttagcctgttgaaacaagccggcgacgttgaagagaaccccggacctatggagaagaatcctttggcagccccattactaatcctctggtttcatcttgactgcgtgagcagcatactgaacgtggaacaaagtcctcagtcactgcatgttcaggagggagacagcaccaatttcacctgcagcttcccttccagcaatttttatgccttacactggtacagaaaggaaactgcaaaaagccccgaggccttgtttgtaatgactttaaatggggatgaaaagaagaaaggacgaataagtgccactcttaataccaaggagggttacagctatttgtacatcaaaggatcccagcctgaagactcagccacatacctctgtgccctgtacaataacaatgacatgcgctttggagcagggaccagactgacagtaaaaccaaacatccagaaccccgaccccgccgtgtaccagctgagggactccaagtccagcgacaagagcgtgtgtctgtttacggacttcgacagccagaccaacgtgagtcaaagcaaggacagcgacgtctacataacggataagaccgtgctggacatgcggagcatggacttcaagagcaacagcgccgtggcctggtccaacaagagcgacttcgcctgcgccaacgccttcaacaacagcatcatccccgaggacaccttcttccccagcagcgacgtgccctgcgacgtgaaactggtggagaagtccttcgagacagacaccaatctgaactttcagaacctgctggtgatcgtgctgcggattctgctgctgaaagtggccggcttcaatctgctgatgaccctgcggctgtggagc

SEQ ID NO: 50 - protein, 611 aa, synthetic construct
MDSWTFCCVSLCILVAKHTDAGVIQSPRHEVTEMGQEVTLRCKPISGHNSLFWYRETMMRGLELLIYFNNNVPIDDSGMPEDRFSAKMPNASFSTLKIQPSEPRDSAVYFCASSPGSTDTQYFGPGTRLTVLEDLNKVFPPEVAVFEPSKAEIAHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGITSASYHQGVLSATILYEILLGKATLYAVLVSALVLMAMVKRKDFGSGRAKRSGSGATNFSLLKQAGDVEENPGPMEKNPLAAPLLILWFHLDCVSSILNVEQSPQSLHVQEGDSTNFTCSFPSSNFYALHWYRKETAKSPEALFVMTLNGDEKKKGRISATLNTKEGYSYLYIKGSQPEDSATYLCALYNNNDMRFGAGTRLTVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSSDVPCDVKLVEKSFETDTNLNFQNLLVIVLRILLLKVAGFNLLMTLRLWS

SEQ ID NO: 51 - protein, 6 aa, synthetic construct
SSNFYA

SEQ ID NO: 52 - protein, 11 aa, synthetic construct
CALYNNNDMRF

SEQ ID NO: 53 - protein, 132 aa, synthetic construct
MEKNPLAAPLLILWFHLDCVSSILNVEQSPQSLHVQEGDSTNFTCSFPSSNFYALHWYRWETAKSPEALFVMTLNGDEKKKGRISATLNTKEGYSYLYIKGSQPEDSATYLCALYNNNDMRFGAGTRLTVKP

SEQ ID NO: 54 - protein, 141 aa, synthetic construct
NIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 55 - protein, 273 aa, synthetic construct
MEKNPLAAPLLILWFHLDCVSSILNVEQSPQSLHVQEGDSTNFTCSFPSSNFYALHWYRWETAKSPEALFVMTLNGDEKKKGRISATLNTKEGYSYLYIKGSQPEDSATYLCALYNNNDMRFGAGTRLTVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 56 - protein, 5 aa, synthetic construct
SGHNS

SEQ ID NO: 57 - protein, 6 aa, synthetic construct
FNNNVP

SEQ ID NO: 58 - protein, 13 aa, synthetic construct
CASSPGSTDTQYF

SEQ ID NO: 59 - protein, 132 aa, synthetic construct
MDSWTFCCVSLCILVAKHTDAGVIQSPRHEVTEMGQEVTLRCKPISGHNSLFWYRQTMMRGLELLIYFNNNVPIDDSGMPEDRFSAKMPNASFSTLKIQPSEPRDSAVYFCASSPGSTDTQYFGPGTRLTVL

SEQ ID NO: 60 - protein, 179 aa, synthetic construct
EDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 61 - protein, 311 aa, synthetic construct
MDSWTFCCVSLCILVAKHTDAGVIQSPRHEVTEMGQEVTLRCKPISGHNSLFWYRQTMMRGLELLIYFNNNVPIDDSGMPEDRFSAKMPNASFSTLKIQPSEPRDSAVYFCASSPGSTDTQYFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 62 - protein, 132 aa, synthetic construct
MEKNPLAAPLLILWFHLDCVSSILNVEQSPQSLHVQEGDSTNFTCSFPSSNFYALHWYRKETAKSPEALFVMTLNGDEKKKGRISATLNTKEGYSYLYIKGSQPEDSATYLCALYNNNDMRFGAGTRLTVKP

SEQ ID NO: 63 - protein, 273 aa, synthetic construct
MEKNPLAAPLLILWFHLDCVSSILNVEQSPQSLHVQEGDSTNFTCSFPSSNFYALHWYRKETAKSPEALFVMTLNGDEKKKGRISATLNTKEGYSYLYIKGSQPEDSATYLCALYNNNDMRFGAGTRLTVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 64 - protein, 132 aa, synthetic construct
MDSWTFCCVSLCILVAKHTDAGVIQSPRHEVTEMGQEVTLRCKPISGHNSLFWYRETMMRGLELLIYFNNNVPIDDSGMPEDRFSAKMPNASFSTLKIQPSEPRDSAVYFCASSPGSTDTQYFGPGTRLTVL

SEQ ID NO: 65 - protein, 311 aa, synthetic construct
MDSWTFCCVSLCILVAKHTDAGVIQSPRHEVTEMGQEVTLRCKPISGHNSLFWYRETMMRGLELLIYFNNNVPIDDSGMPEDRFSAKMPNASFSTLKIQPSEPRDSAVYFCASSPGSTDTQYFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 66 - DNA, 8088 bp, synthetic construct
  source 1..8088: mol_type = other DNA
gctagctggcttgttgtccacaaccattaaaccttaaaagctttaaaagccttatatattcttttttttcttataaaacttaaaaccttagaggctatttaagttgctgatttatattaattttattgttcaaacatgagagcttagtacgtgaaacatgagagcttagtacattagccatgagagcttagtacattagccatgagggtttagttcattaaacatgagagcttagtacattaaacatgagagcttagtacatactatcaacaggttgaactgctgatctgtacagtagaattggtaaagagagttgtgtaaaatattgagttcgcacatcttgttgtctgattattgatttttggcgaaaccatttgatcatatgacaagatgtgtatctaccttaacttaatgattttgataaaaatcattaggtaccaattacattgcttgcaattaaccctttaacggttataaggatctagatgagatagaaagatttggttttcggatttgtgttacataagatgcctaaaataaaaattgagattcaattttttttaaacttttttttaattggtggtaagaatattccctctacctgtttgagagtaatgaaattgtagtatgatttttcaacaaactaaaaaaacaacataaatctcacataataactttatttcaatcacacaattgaataccaataggttgacagtacttaccagcctgcaggtgaaagaccccacctgtaggtttggcaagttagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagcgaaccatcagatgtttccagggtgccccaaggacctgaaatgaccctgtgccttatttgaactaaccaatcagtttgcttcttgcttctgtttgtgtgcttctgctccctgagctcaataaaagagcccacaacccctcacttggtgggccagtcctctgatagactgtgtcccctggatacccgtacggtaccgctagcgccaccatgctgagccccgacctgcctgacagcgcttggaataccagactcctgtgcagagtgatgctgtgcctgcttggagctggaagtgtggctgctggtgtcattcagtccccaaggcacctgatcaaagagaagagagagacagccactctgaagtgctaccccattcctagacacgacacggtctattggtatcagcaaggacctggacaggaccctcagttcctgatcagcttctacgagaagatgcagagcgacaagggcagcatccccgacagattttctgcccagcagttcagcgactaccacagcgagctgaacatgagcagcctggaactgggcgatagcgccctgtacttctgtgcctcttctttcgcacgcctggaaggtcgcgataatgaacaattttttgggccagggacacggctcaccgtgctagaagatctgaacaaggtgttccctccagaggtggccgtgttcgagccttctaaggccgagatcgcccacacacaaaaagccaccctcgtgtgcctggccaccggctttttccccgaccacgtggaactgtcttggtgggtcaacggcaaagaggtgcactccggcgtgtcaacggatccccagcctctgaaagaacagcctgccctgaacgacagccggtactgcctgagctccagactgagagtgtccgccaccttctggcagaacccccggaaccacttcagatgccaggtgcagttttacggcctgagcgagaacgacgagtggacccaggacagagccaagcccgtgacacaaatcgtgtctgccgaagcctggggaagagccgattgcggcatcaccagcgcctcctatcaccagggcgtgctgagcgccacaatcctgtacgaaatcctgctgggcaaggccaccctgtacgccgtgctggtgtctgctctggtgctgatggccatggtcaagcggaaggactttggcagcggcagagccaaaaggtccgggagcggtgcgacaaactttagcctgttgaaacaagccggcgacgttgaagagaaccccggacctatggcctgtcctggcttcctgtgggcccttgtgatcagcacttgcctggaattcagcatggctcagacagtcacccagtctcagcccgaaatgagcgtccaagaggctgaaaccgtgactctgtcttgtacctacgacacctccgagagcgattactacctcttttggtataagcaaccgccgtccaggcaaatgatcctcgtgatccggcaagaagcttacaaacagcagaatgctaccgaaaaccggttctccgtcaattttcagaaagccgctaagagctttagcctgaaaatctccgactctcagctcggcgacgctgctatgtatttctgtgcctaccgcaaaacttcttacgataaagtcatttttgggccagggacaagcttatcagtcattccaaacatccagaaccccgaccccgccgtgtaccagctgagggactccaagtccagcgacaagagcgtgtgtctgtttacggacttcgacagccagaccaacgtgagtcaaagcaaggacagcgacgtctacataacggataagaccgtgctggacatgcggagcatggacttcaagagcaacagcgccgtggcctggtccaacaagagcgacttcgcctgcgccaacgccttcaacaacagcatcatccccgaggacaccttcttccccagcagcgacgtgccctgcgacgtgaaactggtggagaagtccttcgagacagacaccaatctgaactttcagaacctgctggtgatcgtgctgcggattctgctgctgaaagtggccggcttcaatctgctgatgaccctgcggctgtggagcagcagggctaagaggtccggcagcggagccaccaatttttccctgctgaaacaggctggtgacgtggaagaaaaccctggccccatggcgctgcccgtcaccgcgctgctgctgcccctggcgctgctgttacacgccgctcggccagagcttcccacccagggcacattctccaacgtgtccaccaatgtgtcgggaggcggcggatcgtcccagttcagagtgtcccctctggaccgcacctggaacctgggcgagaccgtggagctgaaatgtcaggtcctgctgagcaacccgacctccgggtgcagttggctgttccagccgcgtggtgctgccgcaagccctacgttcctgctttacctgagccagaacaagcccaaggcggccgagggcctggacacccagagattctccggcaagcgcctgggggacacattcgtgcttactttgagcgatttccgcagagagaacgagggctactatttctgttcggcgctgagcaattccatcatgtatttcagccactttgtgccagtgttcctgcctgccaagcctaccacaacaccagctccccgtcccccgactccggcgcctaccatcgcgagtcaaccgttgagcctgaggcctgaggcttgtcggcccgctgcggggggtgccgtccacaccaggggcctcgactttgcgtgcgacatctatatttgggcgcctctggcgggtacctgcggggtgctgctgctgtcattggtgattaccctgtactgcaatcaccgcaaccgccggcgggtctgtaagtgcccacggcctgtggtcaagtccggtgacaaaccgtcgctctcggctcgctacgtgcgcgctaagcgcagcggttccggggccaccaacttttcattgctgaagcaggccggtgatgtggaggagaatccagggcccatgcgccccaggctttggctccttcttgctgctcagctcactgtcttgcatggcaactccgttctgcagcagactcccgcctacatcaaggtgcagacgaacaagatggtgatgctgtcatgcgaggccaagatctctctttcaaatatgagaatttattggctacgacagcgccaggccccctccagcgacagccaccacgagttcctggcgctttgggattctgctaaaggcaccatccatggagaggaggtggaacaggagaagatagctgtcttccgcgacgcatcccgcttcatcctgaacctgaccagcgtgaagccggaggacagcggcatctacttctgtatgatcgttggctcccccgagctgaccttcggcaaaggcacccagctgtccgtggtggacttcctgcccaccacagcccagccaaccaagaaatccaccctcaagaagcgcgtgtgccgactgccccgccctgaaacccagaagggccctctgtgctcccccatcacccttggactgctggtggcgggagtcctggtgctgctcgtatctctgggtgtcgccatccacctgtgctgccgccgccgccgcgcccgcctgaggtttatgaaacagttttacaagtgataaatcgatggaagggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattactagtggctccggtgcccgtcagtgggcagagcgcacatcgcccacagtccccgagaagttgtggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaactagtccagtgtggtggaattctgcagatatcacggctagcgccaccatgggtcgggggctgctcaggggcctgtggccgctgcacatcgtcctgtggacgcgtatcgccagcacgatcccaccgcacgttcagaagtcggtgaataacgacatgatagtcactgacaacaacggtgcagtcaagtttccacaactgtgtaaattttgtgatgtgagattttccacctgtgacaaccagaaatcctgcatgagcaactgcagcatcacctccatctgtgagaagccacaggaagtctgtgtggctgtatggagaaagaatgacgagaacataacactagagacagtttgccatgaccccaagctcccctaccatgactttattctggaagatgctgcttctccaaagtgcattatgaaggagaagaaaaagcctggtgagactttcttcatgtgttcctgtagctctgatgagtgcaatgacaacatcatcttctcagaagaatataacaccagcaatcctgacttgttgctagtcatatttcaagtgacaggcatcagcctcctgccaccactgggagttgccatatctgtcatcatcatcttctactgctaccgcgtgaaccggcagcagaaggctagtggttcaggcgcaacgaatttctctttgctgaagcaggctggggatgtcgaagaaaatccgggtccaatggtgggctcgctcaactgcatcgtagcagtctcccagaatatgggcatcgggaagaacggtgatttcccgtggcccccacttcgcaacgagagccgttatttccaaagaatgactacaacctcctccgtggagggtaagcagaacctggtcatcatggggaagaagacctggttctctatccctgaaaaaaaccgccccctgaagggccgcatcaacctggtgctgagcagggaactcaaggagcctcctcagggcgcgcattttctgagccgctcattggatgacgctctcaaactgaccgaacagccggagctagccaacaaggtggacatggtgtggatcgtcggaggctcctccgtgtacaaggaggccatgaatcaccccggccacttgaagctgttcgtcacccggatcatgcaggacttcgagtcggacacgttctttccagagattgacctggagaagtacaagctgctgcccgagtacccgggagttcttagtgatgtgcaggaggagaaaggcatcaagtacaaatttgaggtgtacgagaagaacgactaacggtccgtcctgaccaatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtatacaggttacctcagtctcctaggtacgtcttatatctatgaaaaaacattcaaaagcacaacatctagaagaacttaccttttttcaccactctattgcaaagatatgtaccgatttctctcgaagtacaaaaaaccgctagttttcaaattcacctcaagactttgaaaaaaaattgaatctgtcaatgtcaaataaaatcagaaacaaatgtcataatgttacgttaatgttgtcaggtcgaaaaataaaattgcaaatagaaattttgttccttttttattggtttttattggtgggaaaaatattccctctaactgcaaaagggttaattatgttagaggtagagtcgac

SEQ ID NO: 67 - protein, 20 aa, synthetic construct
  VARIANT 1..10: note = This region may encompass 0-10 residues
  VARIANT 11..20: note = This region may encompass 0-10 residues
GGGGGGGGGGSSSSSSSSSS

SEQ ID NO: 68 - protein, 10 aa, synthetic construct
GGGGSGGGGS

SEQ ID NO: 69 - protein, 8 aa, synthetic construct
GGGSGGGS

SEQ ID NO: 70 - protein, 11 aa, synthetic construct
GGGSGGGSGGS

SEQ ID NO: 71 - protein, 9 aa, unidentified
LAGLIDADG

SEQ ID NO: 72 - DNA, 20 bp, synthetic construct
  source 1..20: mol_type = other DNA
cggctactacaaccagagcg

SEQ ID NO: 73 - DNA, 20 bp, synthetic construct
  source 1..20: mol_type = other DNA
agatcacactgacctggcag

SEQ ID NO: 74 - DNA, 20 bp, synthetic construct
  source 1..20: mol_type = other DNA
aggtcagtgtgatctccgca

SEQ ID NO: 75 - DNA, 20 bp, synthetic construct
  source 1..20: mol_type = other DNA
ggccacggagcgagacatct

SEQ ID NO: 76 - DNA, 20 bp, synthetic construct
  source 1..20: mol_type = other DNA
gccggcagttagttattgag

SEQ ID NO: 77 - DNA, 20 bp, synthetic construct
  source 1..20: mol_type = other DNA
ctaactgccggctttcggtt

SEQ ID NO: 78 - protein, 90 aa, Homo sapiens
LTDVSPEPLQALLERASATLQDLVFDECGITDDQLLALLPSLSHCSQLTTLSFYGNSISISALQSLLQHLIGLSNLTHVLYPVPLESYED

SEQ ID NO: 79 - protein, 90 aa, Homo sapiens
LVFDECGITDDQLLALLPSLSHCSQLTTLSFYGNSISISALQSLLQHLIGLSNLTHVLYPVPLESYEDIHGTLHLERLAYLHARLRELLC

SEQ ID NO: 80 - protein, 90 aa, Homo sapiens
SQLTTLSFYGNSISISALQSLLQHLIGLSNLTHVLYPVPLESYEDIHGTLHLERLAYLHARLRELLCELGRPSMVWLSANPCPHCGDRTF

SEQ ID NO: 81 - protein, 90 aa, Homo sapiens
NSSTLNGSAAMLDCGRDCYAIRVATALTLMTGLYQVLMGVLRLGFVSAYLSQPLLDGFAMGASVTILTSQLKHLLGVRIPRHQGPGMVVL

SEQ ID NO: 82 - protein, 90 aa, Homo sapiens
ATALTLMTGLYQVLMGVLRLGFVSAYLSQPLLDGFAMGASVTILTSQLKHLLGVRIPRHQGPGMVVLTWLSLLRGAGQANVCDVVTSTVC

SEQ ID NO: 83 - protein, 90 aa, Homo sapiens
SQLKHLLGVRIPRHQGPGMVVLTWLSLLRGAGQANVCDVVTSTVCLAVLLAAKELSDRYRHRLRVPLPTELLVIVVATLVSHFGQLHKRF

SEQ ID NO: 84 - protein, 90 aa, Homo sapiens
LQKRKVVVAKALKQVLQLEEDLQEDEVPLIAIMATGGGTRSMTSMYGHLLGLQKLNLLDCASYITGLSGATWTMATLYRDPDWSSKNLEP

SEQ ID NO: 85 - protein, 90 aa, Homo sapiens
QEDEVPLIAIMATGGGTRSMTSMYGHLLGLQKLNLLDCASYITGLSGATWTMATLYRDPDWSSKNLEPAIFEARRHVVKDKLPSLFPDQL

SEQ ID NO: 86 - protein, 90 aa, Homo sapiens
QEEENEKLRGDARQKLPMDLLVMYGHLLGLQKLNLLDCASYITGRGAPGPPGPRGFKGDMGVKGPVEGTQHDPLSQESEVEEPLSQESSE

SEQ ID NO: 87 - protein, 90 aa, Homo sapiens
LEVYPTSLTKNYLEDSLLKILDWFLKEFPRLKSELEDNIRRLRAMYGHLLGLQKLNLLDCASYITGASLYSQQLRGLRWENGLSLGGRSE

SEQ ID NO: 88 - protein, 90 aa, Homo sapiens
MYGHLLGLQKLSLLDCASYITGSQGSFLGSLWWSVTSVFDEKQQDSYFCDITQVVQIACANTFPEEISFTGCFAQMFFFVFLGTAECYSE

SEQ ID NO: 89 - protein, 90 aa, Homo sapiens
MEFLWAPLLGLCCSLAAADRHTVFWNSSNPKFRNEDYTIHVQLNDYVDIICPHYEDHSVADAAMEQYILYLVEHEEYQLCQPQSKDQVRW

(19) United States
(12) Patent Application Publication
(10) Pub. No.: US 2025/0249039 A1
(43) Pub. Date: Aug. 7, 2025

(54) PRAME IMMUNOGENIC PEPTIDES, BINDING PROTEINS RECOGNIZING PRAME IMMUNOGENIC PEPTIDES, AND USES THEREOF

(71) Applicant: TSCAN Therapeutics, Inc., Waltham, MA (US)

(72) Inventors: Mollie M. JUREWICZ, Auburn, MA (US); Cagan GURER, Chappaqua, NY (US); Gavin MACBEATH, Wakefield, MA (US)

(21) Appl. No.: 19/118,629

(22) PCT Filed: Oct. 5, 2023
(86) PCT No.: PCT/US2023/076068

§ 371 Date: Apr. 4, 2025

Related U.S. Application Data
(60) Provisional application No. 63/413,552, filed on Oct. 5, 2022.
(60) Provisional application No. 63/423,294, filed on Nov. 7, 2022.

(51) Int. Cl. A61K 35/17; A61K 39/00; A61K 39/385; A61K 40/11; A61K 40/32; A61K 40/42; A61P 35/00; C07K 14/47; C07K 16/28; C07K 16/30; C12N 5/0783; C12N 15/85; G01N 33/569; G01N 33/574; G01N 33/68

(52) U.S. Cl. CPC A61K 35/17; A61K 39/00119; A61K 39/385; A61K 40/11; A61K 40/32; A61K 40/421; A61K 40/4271; A61P 35/00; C07K 14/4748; C07K 16/2833; C07K 16/3053; C12N 5/0636; C12N 15/85; G01N 33/56972; G01N 33/5748; G01N 33/6857; A61K 2039/605; C07K 2317/565; C07K 2317/92; G01N 2333/70539

(57) ABSTRACT

Provided herein are FRAME immunogenic peptides, binding proteins recognizing FRAME immunogenic peptides, and uses thereof.

### CROSS-REFERENCE TO RELATED APPLICATIONS

This application claims the benefit of priority to U.S. Provisional Application Ser. No. 63/413,552, filed on 5 Oct. 2022, and U.S. Provisional Application Ser. No. 63/423,294, filed on 7 Nov. 2022; the entire contents of each of said applications are incorporated herein in its entirety by this reference.

### BACKGROUND OF THE INVENTION

The cancer/testis antigen PRAME exemplifies an ideal TCR-T cell therapy target due to its high expression in multiple malignancies and its absence in normal tissues. Initially identified in metastatic cutaneous melanoma (Ikeda et al. (1997) Immunity 6:199-208), PRAME is highly expressed in various additional solid tumors including lung, head & neck, and ovarian cancers. PRAME plays a pivotal role in multiple cellular processes and has been demonstrated to exhibit protumorigenic function primarily through inhibition of retinoic acid receptor signaling (Epping et al. (2005) Cell 122:835-847). Targeting of PRAME in solid tumors, particularly when performed as part of a TCR-T multiplexing strategy, represents a promising therapeutic approach in the treatment of many cancer indications. There is a need for developing PRAME-specific TCR immunotherapy, such as to treat disorders characterized by PRAME expression.

### SUMMARY OF THE INVENTION

The present invention is based, at least in part, on the discovery of PRAME immunogenic peptides and binding proteins recognizing such PRAME immunogenic peptides based on unbiased functional screens used to discover the antigen of TCR clonotypes identified from subjects having disorders associated with PRAME expression (e.g., subjects afflicted with a melanoma, head & neck cancer, lung cancer, leukemia (e.g., leukemia sub-types), ovarian cancer, a renal cell carcinoma (RCC), a breast carcinoma, a cervix carcinoma, or a colon carcinoma, a sarcoma, and a neuroblastoma). The identified TCRs recognized PRAME immunogenic peptides, such as those listed in Table 1, in the context of a variety of HLA alleles (e.g., HLA-A*02:01). PRAME is demonstrated herein to be selectively expressed in cancer and testis tissue, but not in normal somatic tissues, thereby making it an ideal target for ACT. The ability of PRAME binding proteins (e.g., TCRs described herein) to bind PRAME immunogenic peptides and to elicit immune responses that kill cells expressing PRAME (e.g., cancer cells) demonstrates the utility of such binding proteins in a diversity of uses, including methods of diagnosis, prognosis, treatment, and screening of agents relevant for disorders characterized by PRAME expression.

In one aspect, an immunogenic peptide comprising a peptide epitope selected from peptide sequences listed in Table 1, is provided.

In another aspect, an immunogenic peptide consisting of a peptide epitope selected from peptide sequences listed in Table 1, is provided.

Numerous embodiments are further provided that may be applied to any aspect encompassed by the present invention and/or combined with any other embodiment described herein. For example, in one embodiment, the immunogenic peptide is derived from a PRAME protein, optionally wherein the immunogenic peptide is 8, 9, 10, 11, 12, 13, 14, 15, 16, 17, 18, 19, 20, 21, 22, 23, 24, or 25 amino acids in length. In another embodiment, the immunogenic peptide is capable of eliciting an immune response against PRAME and/or PRAME-expressing cells in a subject, optionally wherein the immune response is i) a T cell response and/or a CD8+ T cell response and/or ii) selected from the group consisting of T cell expansion (e.g., proliferation), cytokine release, and/or cytotoxic killing.

In still another aspect, an immunogenic composition comprising at least one immunogenic peptide described herein, is provided.

Numerous embodiments are further provided that may be applied to any aspect encompassed by the present invention and/or combined with any other embodiment described herein. For example, in one embodiment, the immunogenic composition further comprises an adjuvant. In another embodiment, the immunogenic composition is capable of eliciting an immune response against PRAME and/or PRAME-expressing cells in a subject, optionally wherein the immune response is i) a T cell response and/or a CD8+ T cell response and/or ii) selected from the group consisting of T cell expansion (e.g., proliferation), cytokine release, and/or cytotoxic killing.

In yet another aspect, a composition comprising a peptide epitope selected from peptide sequences listed in Table 1, and an MHC molecule, is provided.

Numerous embodiments are further provided that may be applied to any aspect encompassed by the present invention and/or combined with any other embodiment described herein. For example, in one embodiment, the MHC molecule is an MHC multimer, optionally wherein the MHC multimer is a tetramer. In another embodiment, the MHC molecule is an MHC class I molecule. In still another embodiment, the MHC molecule comprises an MHC alpha chain that is an HLA serotype selected from the group consisting of HLA-A*02, HLA-A*03, HLA-A*01, HLA-A*11, HLA-A*24, HLA-B*07, HLA-C*07, HLA-C*01, HLA-C*02, HLA-C*03, HLA-C*04, HLA-C*05, HLA-C*06, HLA-C*08, HLA-C*12, HLA-C*14, HLA-C*15, HLA-C*16, HLA-C*17, and HLA-C*18, optionally wherein the HLA allele is selected from the group consisting of HLA-A*02:01, HLA-A*02:02, HLA-A*02:03, HLA-A*02:04, HLA-A*02:05, HLA-A*02:06, HLA-A*02:07, HLA-A*02:10, HLA-A*02:11, HLA-A*02:12, HLA-A*02:13, HLA-A*02:14, HLA-A*02:16, HLA-A*02:17, HLA-A*02:19, HLA-A*02:20, HLA-A*02:22, HLA-A*02:24, HLA-A*02:30, HLA-A*02:42, HLA-A*02:53, HLA-A*02:60, HLA-A*02:74 allele, HLA-A*03:01, HLA-A*03:02, HLA-A*03:05, HLA-A*03:07, HLA-A*01:01, HLA-A*01:02, HLA-A*01:03, HLA-A*01:16 allele, HLA-A*01:01, HLA-A*11:02, HLA-A*11:03, HLA-A*11:04, HLA-A*11:05, HLA-A*11:19 allele, HLA-A*24:02, HLA-A*24:03, HLA-A*24:05, HLA-A*24:07, HLA-A*24:08, HLA-A*24:10, HLA-A*24:14, HLA-A*24:17, HLA-A*24:20, HLA-A*24:22, HLA-A*24:25, HLA-A*24:26, HLA-A*24:58 allele, HLA-B*07:02, HLA-B*07:04, HLA-B*07:05, HLA-B*07:09, HLA-B*07:10, HLA-B*07:15, HLA-B*07:21, HLA-C*07:02, HLA-C*07:01, HLA-C*04:01, HLA-C*06:02, HLA-C*03:04, HLA-C*05:01, HLA-C*16:01, HLA-C*02:02, HLA-C*03:03, HLA-C*12:03, HLA-C*08:02, HLA-C*01:02, HLA-C*17:01, HLA-C*15:02, HLA-C*14:02, HLA-C*12:02, HLA-C*07:04, HLA-C*08:01, HLA-C*03:02, HLA-C*18:01, HLA-C*15:05, HLA-C*16:02, HLA-C*08:04, HLA-C*03:05, and HLA-C*14:03 allele. In yet another embodiment, the HLA serotype is HLA-A*02, such as HLA-A*02:01.

In another aspect, a stable MHC-peptide complex, comprising an immunogenic peptide described herein in the context of an MHC molecule, is provided.

Numerous embodiments are further provided that may be applied to any aspect encompassed by the present invention and/or combined with any other embodiment described herein. For example, in one embodiment, the MHC molecule is an MHC multimer, optionally wherein the MHC multimer is a tetramer. In another embodiment, the MHC molecule is an MHC class I molecule. In still another embodiment, the MHC molecule comprises an MHC alpha chain that is an HLA serotype selected from the group consisting of HLA-A*02:01, HLA-A*02:02, HLA-A*02:03, HLA-A*02:04, HLA-A*02:05, HLA-A*02:06, HLA-A*02:07, HLA-A*02:10, HLA-A*02:11, HLA-A*02:12, HLA-A*02:13, HLA-A*02:14, HLA-A*02:16, HLA-A*02:17, HLA-A*02:19, HLA-A*02:20, HLA-A*02:22, HLA-A*02:24, HLA-A*02:30, HLA-A*02:42, HLA-A*02:53, HLA-A*02:60, HLA-A*02:74 allele, HLA-A*03:01, HLA-A*03:02, HLA-A*03:05, HLA-A*03:07, HLA-A*01:01, HLA-A*01:02, HLA-A*01:03, HLA-A*01:16 allele, HLA-A*11:01, HLA-A*11:02, HLA-A*11:03, HLA-A*11:04, HLA-A*11:05, HLA-A*11:19 allele, HLA-A*24:02, HLA-A*24:03, HLA-A*24:05, HLA-A*24:07, HLA-A*24:08, HLA-A*24:10, HLA-A*24:14, HLA-A*24:17, HLA-A*24:20, HLA-A*24:22, HLA-A*24:25, HLA-A*24:26, HLA-A*24:58 allele, HLA-B*07:02, HLA-B*07:04, HLA-B*07:05, HLA-B*07:09, HLA-B*07:10, HLA-B*07:15, HLA-B*07:21, HLA-C*07:02, HLA-C*07:01, HLA-C*04:01, HLA-C*06:02, HLA-C*03:04, HLA-C*05:01, HLA-C*16:01, HLA-C*02:02, HLA-C*03:03, HLA-C*12:03, HLA-C*08:02, HLA-C*01:02, HLA-C*17:01, HLA-C*15:02, HLA-C*14:02, HLA-C*12:02, HLA-C*07:04, HLA-C*08:01, HLA-C*03:02, HLA-C*18:01, HLA-C*15:05, HLA-C*16:02, HLA-C*08:04, HLA-C*03:05, and HLA-C*14:03 allele. In yet another embodiment, the peptide epitope and the MHC molecule are covalently linked and/or wherein the alpha and beta chains of the MHC molecule are covalently linked. In another embodiment, the stable MHC-peptide complex comprises a detectable label, optionally wherein the detectable label is a fluorophore.

In still another aspect, an immunogenic composition comprising a stable MHC-peptide complex described herein, and an adjuvant, is provided.

In yet another aspect, an isolated nucleic acid that encodes an immunogenic peptide described herein, or a complement thereof, is provided.

In another aspect, a vector comprising an isolated nucleic acid described herein, is provided.

In still another aspect, a cell that a) comprises an isolated nucleic acid described herein, b) comprises a vector described herein, and/or c) produces one or more immunogenic peptides described herein and/or presents at the cell surface one or more stable MHC-peptide complexes described herein, optionally wherein the cell is genetically engineered, is provided.

In yet another aspect, a device or kit comprising a) one or more immunogenic peptides described herein and/or b) one or more stable MHC-peptide complexes described herein, said device or kit optionally comprising a reagent to detect binding of a) and/or b) to a binding protein, optionally wherein the binding protein is an antibody, an antigen-binding fragment of an antibody, a TCR, an antigen-binding fragment of a TCR, a single chain TCR (scTCR), a chimeric antigen receptor (CAR), or a fusion protein comprising a TCR and an effector domain, is provided.

In another aspect, a method of detecting T cells that bind a stable MHC-peptide complex comprising: a) contacting a sample comprising T cells with a stable MHC-peptide complex described herein; and b) detecting binding of T cells to the stable MHC-peptide complex, optionally further determining the percentage of stable MHC-peptide-specific T cells that bind to the stable MHC-peptide complex, optionally wherein the sample comprises peripheral blood mononuclear cells (PBMCs), is provided.

Numerous embodiments are further provided that may be applied to any aspect encompassed by the present invention and/or combined with any other embodiment described herein. For example, in one embodiment, T cells are CD8+ T cells. In another embodiment, detecting and/or determining is performed using fluorescence activated cell sorting (FACS), enzyme linked immunosorbent assay (ELISA), radioimmune assay (RIA), immunochemically. Western blot, or intracellular flow assay. In still another embodiment, a sample comprises T cells contacted with, or suspected of having been contacted with, one or more PRAME proteins or fragments thereof.

In still another aspect, a method of determining whether a T cell has had exposure to PRAME comprising: a) incubating a cell population comprising T cells with an immunogenic peptide described herein or a stable MHC-peptide complex described herein; and b) detecting the presence or level of reactivity, wherein the presence of or a higher level of reactivity compared to a control level indicates that the T cell has had exposure to PRAME, optionally wherein the cell population comprising T cells is obtained from a subject, is provided.

In yet another aspect, a method for predicting the clinical outcome of a subject afflicted with a disorder characterized by PRAME expression comprising: a) determining the presence or level of reactivity between T cells obtained from the subject and one more immunogenic peptides described herein or one or more stable MHC-peptide complexes described herein; and b) comparing the presence or level of reactivity to that from a control, wherein the control is obtained from a subject having a good clinical outcome, wherein the presence or a higher level of reactivity in the subject sample as compared to the control indicates that the subject has a good clinical outcome, is provided.

In another aspect, a method of assessing the efficacy of a therapy for a disorder characterized by PRAME expression comprising: a) determining the presence or level of reactivity between T cells obtained from the subject and one more immunogenic peptides described herein or one or more stable MHC-peptide complexes described herein, in a first sample obtained from the subject prior to providing at least a portion of the therapy to the subject, and b) determining the presence or level of reactivity between the one more immunogenic peptides described herein, or the one or more stable MHC-peptide complexes described herein, and T cells obtained from the subject present in a second sample obtained from the subject following provision of the therapy to the subject, wherein the presence or a higher level of reactivity in the second sample, relative to the first sample, is an indication that the therapy is efficacious for treating the disorder characterized by PRAME expression in the subject, is provided.

Numerous embodiments are further provided that may be applied to any aspect encompassed by the present invention and/or combined with any other embodiment described herein. For example, in one embodiment, the level of reactivity is indicated by a) the presence of binding and/or b) T cell activation and/or effector function, optionally wherein the T cell activation or effector function is T cell proliferation, killing, or cytokine release. In another embodiment, a method further comprises repeating steps a) and b) at a subsequent point in time, optionally wherein the subject has undergone treatment to ameliorate the disorder characterized by PRAME expression between the first point in time and the subsequent point in time. In still another embodiment. T cell binding, activation, and/or effector function is detected using fluorescence activated cell sorting (FACS), enzyme linked immunosorbent assay (ELISA), radioimmune assay (RIA), immunochemically, Western blot, or intracellular flow assay. In yet another embodiment, a control level is a reference number. In another embodiment, a control level is a level of a subject without the disorder characterized by PRAME expression.

In still another aspect, a method of preventing and/or treating a disorder characterized by PRAME expression in a subject comprising administering to the subject a therapeutically effective amount of a composition described herein.

In yet another aspect, a method of identifying a peptide-binding molecule, or antigen-binding fragment thereof, that binds to a peptide epitope selected from the peptide sequences listed in Table 1 comprising: a) providing a cell presenting a peptide epitope selected from the peptide sequences listed in Table 1 in the context of an MHC molecule on the surface of the cell; b) determining binding of a plurality of candidate peptide-binding molecules or antigen-binding fragments thereof to the peptide epitope in the context of the MHC molecule on the cell; and c) identifying one or more peptide-binding molecules or antigen-binding fragments thereof that bind to the peptide epitope in the context of the MHC molecule, is provided.

Numerous embodiments are further provided that may be applied to any aspect encompassed by the present invention and/or combined with any other embodiment described herein. For example, in one embodiment, a step a) comprises contacting the MHC molecule on the surface of the cell with a peptide epitope selected from the peptide sequences listed in Table 1. In another embodiment, a step a) comprises expressing the peptide epitope selected from the peptide sequences listed in Table 1 in the cell using a vector comprising a heterologous sequence encoding the peptide epitope.

In another aspect, a method of identifying a peptide-binding molecule or antigen-binding fragment thereof that binds to a peptide epitope selected from the peptide sequences listed in Table 1 comprising: a) providing a peptide epitope either alone or in a stable MHC-peptide complex, comprising a peptide epitope selected from the peptide sequences listed in Table 1, either alone or in the context of an MHC molecule; b) determining binding of a plurality of candidate peptide-binding molecules or antigen-binding fragments thereof to the peptide or stable MHC-peptide complex; and c) identifying one or more peptide-binding molecules or antigen-binding fragments thereof that bind to the peptide epitope or the stable MHC-peptide complex, optionally wherein the MHC or MHC-peptide complex is as described herein, is provided.

Numerous embodiments are further provided that may be applied to any aspect encompassed by the present invention and/or combined with any other embodiment described herein. For example, in one embodiment, a plurality of candidate peptide binding molecules comprises an antibody, an antigen-binding fragment of an antibody, a TCR, an antigen-binding fragment of a TCR, a single chain TCR (scTCR), a chimeric antigen receptor (CAR), or a fusion protein comprising a TCR and an effector domain. In another embodiment, a plurality of candidate peptide binding molecules comprises at least 2, 5, 10, 100, 10³, 10⁴, 10⁵, 10⁶, 10⁷, 10⁸, 10⁹, or more, different candidate peptide binding molecules. In still another embodiment, a plurality of candidate peptide binding molecules comprises one or more candidate peptide binding molecules that are obtained from a sample from a subject or a population of subjects; or the plurality of candidate peptide binding molecules comprises one or more candidate peptide binding molecules that comprise mutations in a parent scaffold peptide binding molecule obtained from a sample from a subject. In yet another embodiment, a subject or population of subjects are a) not afflicted with a disorder characterized by PRAME expression and/or have recovered from a disorder characterized by PRAME expression, or b) are afflicted with a disorder characterized by PRAME expression. In another embodiment, a subject or population of subjects has been administered a composition described herein. In still another embodiment, a subject is an animal model of a disorder characterized by PRAME expression and/or a mammal, optionally wherein the mammal is a human, a primate, or a rodent. In yet another embodiment, a subject is an animal model of a disorder characterized by PRAME expression, an HLA-transgenic mouse, and/or a human TCR transgenic mouse. In another embodiment, a sample comprises peripheral blood mononuclear cells (PBMCs). T cells, and/or CD8+ memory T cells.

In still another aspect, a peptide-binding molecule or antigen-binding fragment thereof identified according to a method described herein, optionally wherein the peptide-binding molecule or antigen-binding fragment thereof is an antibody, an antigen-binding fragment of an antibody, a TCR, an antigen-binding fragment of a TCR, a single chain TCR (scTCR), a chimeric antigen receptor (CAR), or a fusion protein comprising a TCR and an effector domain, is provided.

In yet another aspect, a method of treating a disorder characterized by PRAME expression in a subject comprising administering to the subject a therapeutically effective amount of genetically engineered T cells that express a peptide-binding molecule or antigen-binding fragment thereof that i) binds to a peptide epitope selected from the sequences listed in Table 1, ii) is identified according to a method described herein, and/or iii) binds to a stable MHC-peptide complex comprising a peptide epitopes selected from the sequences listed in Table 1 in the context of an MHC molecule, optionally wherein the peptide-binding molecule or antigen-binding fragment thereof is an antibody, an antigen-binding fragment of an antibody, a TCR, an antigen-binding fragment of a TCR, a single chain TCR (scTCR), a chimeric antigen receptor (CAR), or a fusion protein comprising a TCR and an effector domain, optionally wherein the MHC or MHC-peptide complex is as described herein, is provided.

Numerous embodiments are further provided that may be applied to any aspect encompassed by the present invention and/or combined with any other embodiment described herein. For example, in one embodiment. T cells are isolated from a) the subject, b) a donor not afflicted with the disorder characterized by PRAME expression, or c) a donor recovered from a disorder characterized by PRAME expression.

In another aspect, a method of treating a disorder characterized by PRAME expression in a subject comprising transfusing antigen-specific T cells to the subject, wherein the antigen-specific T cells are generated by: a) stimulating immune cells from a subject with a composition described herein; and b) expanding antigen-specific T cells in vitro or ex vivo, optionally i) isolating immune cells from the subject before stimulating the immune cells and/or ii) wherein the immune cells comprise PBMCs, T cells, CD8+ T cells, naive T cells, central memory T cells, and/or effector memory T cells, is provided.

Numerous embodiments are further provided that may be applied to any aspect encompassed by the present invention and/or combined with any other embodiment described herein. For example, in one embodiment, agents are placed in contact under conditions and for a time suitable for the formation of at least one immune complex between the peptide epitope, immunogenic peptide, stable MHC-peptide complex, T cell receptor, and/or immune cells. In another embodiment, a peptide epitope, immunogenic peptide, stable MHC-peptide complex, and/or T cell receptor is expressed by cells and the cells are expanded and/or isolated during one or more steps. In still another embodiment, a disorder characterized by PRAME expression is a cancer or relapse thereof, optionally wherein the cancer is selected from the group consisting of melanoma, head & neck cancer, lung cancer, leukemia (e.g., leukemia sub-types), ovarian cancer, a renal cell carcinoma (RCC), a breast carcinoma, a cervix carcinoma, or a colon carcinoma, a sarcoma, and a neuroblastoma. In yet another embodiment, a subject is an animal model of a disorder characterized by PRAME expression and/or a mammal, optionally wherein the mammal is a human, a primate, or a rodent.

In still another aspect, a binding protein that binds a polypeptide comprising an immunogenic peptide sequence described herein, an immunogenic peptide described herein, and/or the stable MHC-peptide complex described herein, optionally wherein the binding protein is an antibody, an antigen-binding fragment of an antibody, a TCR, an antigen-binding fragment of a TCR, a single chain TCR (scTCR), a chimeric antigen receptor (CAR), or a fusion protein comprising a TCR and an effector domain, is provided.

Numerous embodiments are further provided that may be applied to any aspect encompassed by the present invention and/or combined with any other embodiment described herein. For example, in one embodiment, a binding protein comprises: a) a T cell receptor (TCR) alpha chain CDR sequence with at least about 80% identity to a TCR alpha chain CDR sequence selected from the group consisting of TCR alpha chain CDR sequences listed in Table 2; and/or b) a TCR beta chain CDR sequence with at least about 80% identity to a TCR beta chain CDR sequence selected from the group consisting of TCR beta chain CDR sequences listed in Table 2, wherein the binding protein is capable of binding to a PRAME immunogenic peptide-MHC (pMHC) complex, optionally wherein the binding affinity has a K_{d} less than or equal to about 5×10⁻⁴ M. In another embodiment, a binding protein comprises: a) a TCR alpha chain variable (V_{α}) domain sequence with at least about 80% identity to a TCR V_{α} domain sequence selected from the group consisting of TCR V_{α} domain sequences listed in Table 2; and/or b) a TCR beta chain variable (V_{β}) domain sequence with at least about 80% identity to a TCR V_{β} domain sequence selected from the group consisting of TCR V_{β} domain sequences listed in Table 2, wherein the binding protein is capable of binding to a PRAME immunogenic peptide-MHC (pMHC) complex, optionally wherein the binding affinity has a K_{d} less than or equal to about 5×10⁻⁴ M. In still another embodiment, a binding protein comprises: a) a TCR alpha chain sequence with at least about 80% identity to a TCR alpha chain sequence selected from the group consisting of TCR alpha chain sequences listed in Table 2; and/or b) a TCR beta chain sequence with at least about 80% identity to a TCR beta chain sequence selected from the group consisting of TCR beta chain sequences listed in Table 2, wherein the binding protein is capable of binding to a PRAME immunogenic peptide-MHC (pMHC) complex, optionally wherein the binding affinity has a K_{d} less than or equal to about 5×10⁻⁴ M. In yet another embodiment, a binding protein comprises: a) a TCR alpha chain CDR sequence selected from the group consisting of TCR alpha chain CDR sequences listed in Table 2; and/or b) a TCR beta chain CDR sequence selected from the group consisting of TCR beta chain CDR sequences listed in Table 2, wherein the binding protein is capable of binding to a PRAME immunogenic peptide-MHC (pMHC) complex, optionally wherein the binding affinity has a K_{d} less than or equal to about 5×10⁻⁴ M. In yet another embodiment, a binding protein comprises: a) a TCR alpha chain variable (V_{α}) domain sequence selected from the group consisting of TCR V_{α} domain sequences listed in Table 2; and/or b) a TCR beta chain variable (V_{β}) domain sequence selected from the group consisting of TCR V_{β} domain sequences listed in Table 2, wherein the binding protein is capable of binding to a PRAME immunogenic peptide-MHC (pMHC) complex, optionally wherein the binding affinity has a K_{d} less than or equal to about 5×10⁻⁴ M, is provided. In another embodiment, a binding protein comprises: a) a TCR alpha chain sequence selected from the group consisting of TCR alpha chain sequences listed in Table 2; and/or b) a TCR beta chain sequence selected from the group consisting of TCR beta chain sequences listed in Table 2, wherein the binding protein is capable of binding to a PRAME immunogenic peptide-MHC (pMHC) complex, optionally wherein the binding affinity has a K_{d} less than or equal to about 5×10⁻⁴ M, is provided. In another embodiment, 1) a TCR alpha chain CDR, TCR V_{α} domain, and/or TCR alpha chain is encoded by a TRAV, TRAJ, and/or TRAC gene or fragment thereof selected from the group of TRAV, TRAJ, and TRAC genes listed in Table 2, and/or 2) a TCR beta chain CDR, TCR V_{β} domain, and/or TCR beta chain is encoded by a TRBV, TRBJ, and/or TRBC gene or fragment thereof selected from the group of TRBV. TRBJ, and TRBC genes listed in Table 2, and/or 3) each CDR of the binding protein has up to five amino acid substitutions, insertions, deletions, or a combination thereof as compared to the cognate reference CDR sequence listed in Table 2. In still another embodiment, a binding protein is chimeric, humanized, or human. In yet another embodiment, a binding protein comprises a binding domain having a transmembrane domain, and an effector domain that is intracellular. In another embodiment, a TCR alpha chain and a TCR beta chain are covalently linked, optionally wherein the TCR alpha chain and the TCR beta chain are covalently linked through a linker peptide. In still another embodiment, a TCR alpha chain and/or a TCR beta chain are covalently linked to a moiety, optionally wherein the covalently linked moiety comprises an affinity tag or a label. In yet another embodiment, an affinity tag is selected from the group consisting of aCD34 enrichment tag, glutathione-S-transferase (GST), calmodulin binding protein (CBP), protein C tag, Myc tag, HaloTag, HA tag, Flag tag, His tag, biotin tag, and V5 tag, and/or wherein the label is a fluorescent protein. In another embodiment, a covalently linked moiety is selected from the group consisting of an inflammatory agent, cytokine, toxin, cytotoxic molecule, radioactive isotope, or antibody or antigen-binding fragment thereof. In still another embodiment, a binding protein binds to the pMHC complex on a cell surface. In yet another embodiment, an MHC or MHC-peptide complex is as described herein. In another embodiment, binding of a binding protein to the PRAME peptide-MHC (pMHC) complex elicits an immune response, optionally wherein the immune response is i) a T cell response and/or a CD8+ T cell response and/or ii) selected from the group consisting of T cell expansion, cytokine release, and/or cytotoxic killing. In still another embodiment, a binding protein is capable of specifically and/or selectively binding to a PRAME immunogenic peptide-MHC (pMHC) complex with a K_{d} less than or equal to about 1×10⁻⁴ M, less than or equal to about 5×10⁻⁵ M, less than or equal to about 1×10⁻⁵ M, less than or equal to about 5×10⁻⁶ M, less than or equal to about 1×10⁻⁶ M, less than or equal to about 5×10⁻⁷ M, less than or equal to about 1×10⁻⁷ M, less than or equal to about 5×10⁻⁸ M, less than or equal to about 1×10⁻⁸ M, less than or equal to about 5×10⁻⁹ M, less than or equal to about 1×10⁻⁹ M, less than or equal to about 5×10⁻¹⁰ M, less than or equal to about 1×10⁻¹⁰ M, less than or equal to about 5×10⁻¹¹ M, less than or equal to about 1×10⁻¹¹ M, less than or equal to about 5×10⁻¹² M, or less than or equal to about 1×10⁻¹² M. In yet another embodiment, a binding protein has a higher binding affinity to the peptide-MHC (pMHC) than does a known T-cell receptor, optionally wherein the higher binding affinity is at least 1.05-fold higher. In another embodiment, a binding protein induces higher T cell expansion, cytokine release, and/or cytotoxic killing than does a known T-cell receptor when contacted with target cells with a heterozygous expression of PRAME, optionally wherein the induction is at least 1.05-fold higher. As used herein, references to fold changes, in some embodiments, may be in comparison to any reference modality of interest, such as comparison to a different binding protein; comparison to the same binding protein under different context like expression of the same binding protein in a different immune cell, at a different level, in combination with other agents described herein; and the like. In still another embodiment, cytotoxic killing is of a target cancer cell. In yet another embodiment, cancer is selected from the group consisting of melanoma, head & neck cancer, lung cancer, leukemia (e.g., leukemia sub-types), ovarian cancer, a renal cell carcinoma (RCC), a breast carcinoma, a cervix carcinoma, or a colon carcinoma, a sarcoma, and a neuroblastoma. In another embodiment, a binding protein does not bind to a peptide-MHC (pMHC) complex comprising a PLA2G4E, EFNA1, and/or SLC26A1 peptide epitope. These genes are well-known and are art-recognized to be annotated according to the following NCBI Gene ID numbers, each of which is available on the World Wide Web at ncbi.nlm.nih.gov/gene: PLA2G4E: Gene ID 123745; EFNA1: Gene ID 1942; and SLC26A1: Gene ID 10861.

In yet another aspect, a TCR alpha chain and/or beta chain selected from the group consisting of TCR alpha chain and beta chain sequences listed in Table 2, is provided.

In another aspect, an isolated nucleic acid molecule i) that hybridizes, under stringent conditions, with the complement of a nucleic acid encoding a polypeptide selected from the group consisting of polypeptide sequences listed in Table 2, ii) a sequence with at least about 80% homology to a nucleic acid encoding a polypeptide selected from the group consisting of the polypeptide sequences listed in Table 2, and/or iii) ii) a sequence with at least about 80% homology to a nucleic acid encoding listed in Table 2, optionally wherein the isolated nucleic acid molecule comprises 1) a TRAV, TRAJ, and/or TRAC gene or fragment thereof selected from the group of TRAV, TRAJ, and TRAC genes listed in Table 2 and/or 2) a TRBV, TRBJ, and/or TRBC gene or fragment thereof selected from the group of TRBV, TRBJ, and TRBC genes listed in Table 2, is provided.

Numerous embodiments are further provided that may be applied to any aspect encompassed by the present invention and/or combined with any other embodiment described herein. For example, in one embodiment, a nucleic acid is codon optimized for expression in a host cell.

In still another aspect, a vector comprising an isolated nucleic acid described herein, optionally wherein i) the vector is a cloning vector, expression vector, or viral vector and/or ii) the vector comprises a vector sequence listed in Table 3, is provided.

Numerous embodiments are further provided that may be applied to any aspect encompassed by the present invention and/or combined with any other embodiment described herein. For example, in one embodiment, a vector further comprises a nucleic acid sequence encoding CD8a CD8β, a dominant negative TGFβ receptor II (DN-TGFβRII), selectable protein marker, optionally wherein the selectable protein marker is dihydrofolate reductase (DHFR). In another embodiment, a nucleic acid sequence encoding CD8α, CD8β, DN-TGFβRII, and/or the selectable protein marker is operably linked to a nucleic acid encoding a tag. In still another embodiment, a nucleic acid encoding a tag is at the 5′ upstream of the nucleic acid sequence encoding CD8α, CD8β, the DN-TGFβRII, and/or the selectable protein such that the tag is fused to the N-terminus of CD8α, CD8β, the DN-TGFβRII, and/or the selectable protein marker. In yet another embodiment, a tag is a CD34 enrichment tag. In another embodiment, an isolated nucleic acid described herein, either alone or in combination with a nucleic acid sequence encoding CD8α, CD8β, the DN-TGFβRII, and/or the selectable protein marker are interconnected with an internal ribosome entry site or a nucleic acid sequence encoding a self-cleaving peptide. In still another embodiment, a self-cleaving peptide is P2A, E2A, F2A or T2A.

In yet another aspect, a host cell which comprises an isolated nucleic acid described herein, comprises a vector described herein, and/or expresses a binding protein described herein, optionally wherein the cell is genetically engineered, is provided.

Numerous embodiments are further provided that may be applied to any aspect encompassed by the present invention and/or combined with any other embodiment described herein. For example, in one embodiment, a host cell comprises a chromosomal gene knockout of a TCR gene, an HLA gene, or both. In another embodiment, a host cell comprises a knockout of an HLA gene selected from an α1 macroglobulin gene, α2 macroglobulin gene, α3 macroglobulin gene, β1 microglobulin gene, β2 microglobulin gene, and combinations thereof. In still another embodiment, a host cell comprises a knockout of a TCR gene selected from a TCR α variable region gene, TCR β variable region gene, TCR constant region gene, and combinations thereof. In yet another embodiment, a host cell expresses CD8α, CD8β, a DN-TGFβRII, and/or a selectable protein marker, optionally wherein the selectable protein marker is DHFR, and further optionally wherein the CD8α, CD8β, the DN-TGFβRII, and/or the selectable protein marker is fused to a CD34 enrichment tag. In another embodiment, host cells are enriched using the CD34 enrichment tag. In still another embodiment, a host cell is a hematopoietic progenitor cell, peripheral blood mononuclear cell (PBMC), cord blood cell, or immune cell. In yet another embodiment, an immune cell is a T cell, cytotoxic lymphocyte, cytotoxic lymphocyte precursor cell, cytotoxic lymphocyte progenitor cell, cytotoxic lymphocyte stem cell, CD4⁺ T cell, CD8⁺ T cell. CD4/CD8 double negative T cell, gamma delta (γδ) T cell, natural killer (NK) cell, NK-T cell, dendritic cell, or a combination thereof. In yet another embodiment a T cell is a naive T cell, central memory T cell, effector memory T cell, or a combination thereof. In another embodiment, a T cell is a primary T cell or a cell of a T cell line. In still another embodiment, a T cell does not express or has a lower surface expression of an endogenous TCR. In yet another embodiment, a host cell is capable of producing a cytokine or a cytotoxic molecule when contacted with a target cell that comprises a peptide-MHC (pMHC) complex comprising a PRAME peptide epitope in the context of an MHC molecule. In another embodiment, a host cell is contacted with the target cell in vitro, ex vivo, or in vivo. In still another embodiment, a cytokine is TNF-α, IL-2, and/or IFN-γ. In yet another embodiment, a cytotoxic molecule is perforins and/or granzymes, optionally wherein the cytotoxic molecule is granzyme B. In another embodiment, a host cell is capable of producing a higher level of cytokine or a cytotoxic molecule when contacted with a target cell with a heterozygous expression of PRAME. In still another embodiment, a host cell is capable of producing an at least 1.05-fold higher level of cytokine or a cytotoxic molecule. In yet another embodiment, a host cell is capable of killing a target cell that comprises a peptide-MHC (pMHC) complex comprising the PRAME peptide epitope in the context of an MHC molecule. In another embodiment, killing is determined by a killing assay. In still another embodiment, a ratio of the host cell and the target cell in the killing assay is from 20:1 to 1:4. In yet another embodiment, a target cell is a target cell pulsed with 1 μg/mL to 50 μg/mL of PRAME peptide, optionally wherein the target cell is a cell monoallelic for an MHC matched to the PRAME peptide. In another embodiment, a host cell is capable of killing a higher number of target cells when contacted with target cells with a heterozygous expression of PRAME, optionally wherein the cell killing is at least 1.05-fold higher. In still another embodiment, a target cell is cell line (such as Hs695T, A375, or NCI-H1563) or a primary cell, optionally wherein the target cell is selected from the group consisting of a HEK293 derived cell line, a cancer cell line, a primary cancer cell, a transformed cell line, and an immortalized cell line. In yet another embodiment, a PRAME immunogenic peptide is as described herein and/or wherein an MHC or MHC-peptide complex is as described herein. In another embodiment, a host cell does not induce T cell expansion, cytokine release, or cytotoxic killing when contact with a target cell that comprises a peptide-MHC (pMHC) complex comprising a PLA2G4E, EFNA1, and/or SLC26A1 peptide epitope. In still another embodiment, a host cell does not express PRAME antigen, is not recognized by a binding protein described herein, is not of serotype HLA-A*02, and/or does not express an HLA-A*02 allele.

In another aspect, a population of host cells described herein, is provided.

In still another aspect, a composition comprising a) a binding protein described herein, b) an isolated nucleic acid described herein. c) a vector described herein, d) a host cell described herein, and/or e) a population of host cells described herein, and a carrier, is provided.

In yet another aspect, a device or kit comprising a) a binding protein described herein, b) an isolated nucleic acid described herein, c) a vector described herein, d) a host cell described herein, and/or e) a population of host cells described herein, said device or kit optionally comprising a reagent to detect binding of a), d) and/or e) to a pMHC complex, is provided.

In another aspect, a method of producing a binding protein described herein, wherein the method comprises the steps of: (i) culturing a transformed host cell which has been transformed by a nucleic acid comprising a sequence encoding a binding protein described herein under conditions suitable to allow expression of said binding protein; and (ii) recovering the expressed binding protein, is provided.

In still another aspect, a method of producing a host cell expressing a binding protein described herein, wherein the method comprises the steps of: (i) introducing a nucleic acid comprising a sequence encoding a binding protein described herein into the host cell; and (ii) culturing the transformed host cell under conditions suitable to allow expression of said binding protein, is provided.

In yet another aspect, a method of detecting the presence or absence of a PRAME antigen and/or a cell expressing PRAME, optionally wherein the cell is a hyperproliferative cell, comprising detecting the presence or absence of said PRAME antigen in a sample by use of at least one binding protein described herein, at least one host cell described herein, or a population of host cells described herein, wherein detection of the PRAME antigen is indicative of the presence of a PRAME antigen and/or cell expressing PRAME, is provided.

Numerous embodiments are further provided that may be applied to any aspect encompassed by the present invention and/or combined with any other embodiment described herein. For example, in one embodiment, at least one binding protein, or at least one host cell, forms a complex with the PRAME peptide in the context of an MHC molecule, and the complex is detected in the form of fluorescence activated cell sorting (FACS), enzyme linked immunosorbent assay (ELISA), radioimmune assay (RIA), immunochemically, Western blot, or intracellular flow assay. In another embodiment, a method further comprises obtaining a sample from a subject.

In another aspect, a method of detecting the level of a disorder characterized by PRAME expression in a subject, comprising: a) contacting a sample obtained from the subject with at least one binding protein described herein, at least one host cell described herein, or a population of host cells described herein; and b) detecting the level of reactivity, wherein the presence or a higher level of reactivity compared to a control level indicates the level of the disorder characterized by PRAME expression in the subject, is provided.

Numerous embodiments are further provided that may be applied to any aspect encompassed by the present invention and/or combined with any other embodiment described herein. For example, in one embodiment, a control level is a reference number. In another embodiment, a control level is a level from a subject without the disorder characterized by PRAME expression.

In still another aspect, a method for monitoring the progression of a disorder characterized by PRAME expression in a subject, the method comprising: a) detecting in a subject sample the presence or level of reactivity between a sample obtained from the subject and at least one binding protein described herein, at least one host cell described herein, or a population of host cells described herein; b) repeating step a) at a subsequent point in time; and c) comparing the level of PRAME or the cell of interest expressing PRAME detected in steps a) and b) to monitor the progression of the disorder characterized by PRAME expression in the subject, wherein an absent or reduced PRAME level or the cell of interest expressing PRAME detected in step b) compared to step a) indicates an inhibited progression of the disorder characterized by PRAME expression in the subject and a presence or increased PRAME level or the cell of interest expressing PRAME detected in step b) compared to step a) indicates a progression of the disorder characterized by PRAME expression in the subject, is provided.

Numerous embodiments are further provided that may be applied to any aspect encompassed by the present invention and/or combined with any other embodiment described herein. For example, in one embodiment, a subject has undergone treatment to treat a disorder characterized by PRAME expression between the first point in time and the subsequent point in time.

In yet another aspect, a method for predicting the clinical outcome of a subject afflicted with a disorder characterized by PRAME expression comprising: a) determining the presence or level of reactivity between a sample obtained from the subject and at least one binding protein described herein, at least one host cell described herein, or a population of host cells described herein; and b) comparing the presence or level of reactivity to that from a control, wherein the control is obtained from a subject having a good clinical outcome; wherein the absence or a reduced level of reactivity in the subject sample as compared to the control indicates that the subject has a good clinical outcome, is provided.

In another aspect, a method of assessing the efficacy of a therapy for a disorder characterized by PRAME expression comprising: a) determining the presence or level of reactivity between a sample obtained from the subject and at least one binding protein described herein, at least one host cell described herein, or a population of host cells described herein, in a first sample obtained from the subject prior to providing at least a portion of the therapy for the disorder characterized by PRAME expression to the subject, and b) determining the presence or level of reactivity between a sample obtained from the subject and at least one binding protein described herein, at least one host cell described herein, or a population of host cells described herein, in a second sample obtained from the subject following provision of the therapy for the disorder characterized by PRAME expression, wherein the absence or a reduced level of reactivity in the second sample, relative to the first sample, is an indication that the therapy is efficacious for treating the disorder characterized by PRAME expression in the subject, and wherein the presence or an increased level of reactivity in the second sample, relative to the first sample, is an indication that the therapy is not efficacious for treating the disorder characterized by PRAME expression in the subject, is provided.

Numerous embodiments are further provided that may be applied to any aspect encompassed by the present invention and/or combined with any other embodiment described herein. For example, in one embodiment, a level of reactivity is indicated by a) the presence of binding and/or b) T cell activation and/or effector function, optionally wherein the T cell activation or effector function is T cell proliferation, killing, or cytokine release. In another embodiment, a T cell binding, activation, and/or effector function is detected using fluorescence activated cell sorting (FACS), enzyme linked immunosorbent assay (ELISA), radioimmune assay (RIA), immunochemically. Western blot, or intracellular flow assay.

In still another aspect, a method of preventing and/or treating a disorder characterized by PRAME expression comprising contacting target cells expressing PRAME with a therapeutically effective amount of a composition comprising cells expressing at least one binding protein described herein, optionally wherein the composition is administered to a subject, is provided.

Numerous embodiments are further provided that may be applied to any aspect encompassed by the present invention and/or combined with any other embodiment described herein. For example, in one embodiment, a cell is an allogeneic cell, syngeneic cell, or autologous cell. In another embodiment, a cell is host cell described herein or a population of host cells described herein. In still another embodiment, a target cell is a cancer cell expressing PRAME. In yet another embodiment, a cell composition further comprises a pharmaceutically acceptable carrier. In another embodiment, a cell composition induces an immune response against the target cell expressing PRAME in the subject. In still another embodiment, a cell composition induces an antigen-specific T cell immune response against the target cell expressing PRAME in the subject. In yet another embodiment, an antigen-specific T cell immune response comprises at least one of a CD4⁺ helper T lymphocyte (Th) response and a CD8+ cytotoxic T lymphocyte (CTL) response. In another embodiment, a method further comprises administering at least one additional treatment for the disorder characterized by PRAME expression, optionally wherein the at least one additional treatment for the disorder characterized by PRAME expression is administered concurrently or sequentially with the composition. In still another embodiment, a disorder characterized by PRAME expression is a cancer or relapse thereof, optionally wherein the cancer is selected from the group consisting of melanoma, head & neck cancer, lung cancer, leukemia (e.g., leukemia sub-types), ovarian cancer, a renal cell carcinoma (RCC), a breast carcinoma, a cervix carcinoma, or a colon carcinoma, a sarcoma, and a neuroblastoma. In yet another embodiment, a subject is an animal model of a disorder characterized by PRAME expression and/or a mammal, optionally wherein the mammal is a human, a primate, or a rodent.

### BRIEF DESCRIPTION OF THE DRAWINGS

FIG. 1 shows the PRAME₄₂₅₋₄₃₃ peptide sequence.

FIG. 2A and FIG. 2B show that 392 PRAME₄₂₅₋₄₃₃-specific TCRs were discovered using the ReceptorScan platform. FIG. 2A shows expansion of target-specific CD8⁺ T cells. Briefly, CD14⁺ monocytes were isolated from PBMCs of HLA-A*02:01 healthy donors on day −4 and differentiated to mature DCs. On day −1, naïve CD8 T cells were isolated from autologous PBMCs and rested overnight. Co-culture of naïve CD8 T cells and DCs was performed following 3 hours pulsing of DCs with 1 μg/mL PRAME₄₂₅₋₄₃₃ (SLLQHLIGL) as part of the multiplexed ReceptorScan screens, followed by a 10-day cell expansion phase. FIG. 2B shows isolation and single-cell sequencing of CD8⁺ cells. Dextramer staining was performed with HLA-A*02:01-specific PRAME₄₂₅₋₄₃₃ (SLLQHLIGL) dextramer to identify clones. DNA-barcoded dextramers were used to isolate PRAME₄₂₅₋₄₃₃-specific cells. Sequencing of isolated T cells and pairing of TCR alpha and beta chains was performing using the 10× Genomics platform.

FIG. 3 shows that screening of PRAME₄₂₅₋₄₃₃ TCRs identified 7 TCRs with cytotoxic activity favorable to comparator TCR. Pan T cells were transduced to express 392 PRAME₄₂₅₋₄₃₃-specific TCRs individually, and engineered T cells were then co-cultured with NucLight™ Red-labeled T2 target cells pulsed with 1 ng/mL PRAME₄₂₅₋₄₃₃ peptide. Target cell survival was quantified by time-dependent imaging as a readout of T cell cytotoxicity. Non-transduced cells (NTD) served as a control. Seven (7) out of 392 TCRs were selected for further evaluation for surface expression and cytotoxic potential against PRAME-expressing cell lines.

FIG. 4A and FIG. 4B show that TCRs 366 and 358 displayed cytotoxicity against endogenously expressing cell lines favorably to comparator TCR. Pan T cells from an HLA-A*02:01-positive healthy donor were transduced to express 7 PRAME₄₂₅₋₄₃₃ TCRs that were selected from initial cytotoxicity screens using pulsed T2 cells as targets. Comparator TCRs were similarly expressed. Three (3) TCRs were shown to bind PRAME₄₂₅₋₄₃₃ (SLLQHLIGL) dextramer and were evaluated further in an in vitro cytotoxicity assay, in which they were compared to comparator TCRs (comparator AE: comparator affinity-enhanced). FIG. 4A shows surface expression of the 7 TCRs and comparator TCRs as assessed by A*02:01-restricted PRAME₄₂₅₋₄₃₃ (SLLQHLIGL) dextramer staining, gated on live cells. FIG. 4B shows cytotoxic responses of these TCRs to target cell lines Hs695T, A375, and pulsed T2 cells. Engineered T cells were co-cultured with NucLight™ Red-labeled target cell lines at indicated E:T ratios, and their survival was quantified on an IncuCyte® instrument as a readout of cytotoxicity of the T cells. TCRs 366 and 358 showed favorable activity to the comparator TCR, particularly in control of A375 cell growth, in which PRAME expression is lower.

FIG. 5A-FIG. 5I show results of functional evaluation of PRAME₄₂₅₋₄₃₃ TCRs. Pan T cells isolated from three HLA-A*02:01-positive healthy donor PBMCs were transduced to express PRAME₄₂₅₋₄₃₃-specific TCRs 366 and 358, as well as the comparator TCRs, and T cells were assessed for functional responses against target cells that expressed HLA-A*02:01 and varying levels of PRAME, as well as a PRAME-negative control line. FIG. 5A shows expression of PRAME₄₂₅₋₄₃₃-specific TCRs, as assessed by A*02:01-restricted PRAME₄₂₅₋₄₃₃ (SLLQHLIGL) dextramer staining, gated on live cells (Comparator AE: comparator affinity-enhanced). FIG. 5B-FIG. 5I show results of functional responses of the PRAME₄₂₅₋₄₃₃-specific TCRs to HLA-A*02:01⁺ PRAME⁺ target cell lines Hs695T (FIG. 5B, FIG. 5C), A375 (FIG. 5D, FIG. 5E), NCI-H1563 (FIG. 5F, FIG. 5G), and to the HLA-A*02:01-PRAME-negative control cell line 647V (FIG. 5H. FIG. 5I). Engineered T cells were co-cultured with NucLight Red-labeled target cell lines at indicated E:T ratios, and their survival was quantified on an IncuCyte® as a readout of T cell cytotoxicity. Production of IFN-γ, IL-2, TNF-α, and granzyme B was measured in co-culture supernatants at 24 hour (E:T 1:1) (Comparator AE: comparator affinity-enhanced). FIG. 5A shows expression of PRAME₄₂₅₋₄₃₃ TCRs 366 and 358 on the surface of engineered T cells in three healthy donors. FIG. 5B shows T cell cytotoxicity of Hs695T (HLA-A*02:01⁺PRAME⁺) targets at E:T of 5:1. FIG. 5C shows T cell cytokine production in response to Hs695T (HLA-A*02:01⁺PRAME⁺) targets. FIG. 5D shows T cell cytotoxicity of A375 (HLA-A*02:01⁺PRAME⁺) targets at E:T of 5:1. FIG. 5E shows T cell cytokine production in response to A375 (HLA-A*02:01⁺PRAME⁺) targets. FIG. 5F shows T cell cytotoxicity of NCI-H1563 (HLA-A*02:01⁺PRAME⁺) targets at E:T of 5:1. FIG. 5G shows T cell cytokine production in response to NCI-H1563 (HLA-A*02:01⁺PRAME⁺) targets. FIG. 5H shows T cell cytotoxicity of 647V (HLA-A*02:01⁺PRAME⁻) targets at E:T of 5:1. FIG. 5I shows T cell cytokine production in response to 647V (HLA-A*02:01⁺PRAME⁻) targets.

FIG. 6 shows that the EC50 of TCR 366 was favorable to comparator TCR. EC50 values were determined following pulsing of Nuclight Red-labeled T2 cells with a 10-fold serial dilution of PRAME₄₂₅₋₄₃₃ peptide from 1 μM to 10 fM. Pulsed T2 cells were then co-cultured with T cells at a 5:1 ratio of T cells to targets, and target cell survival was measured in an Incucyte® instrument as a readout of cytotoxicity. EC50 calculations were performed by fitting area-under-the-curve (AUC) data using Prism software.

FIG. 7 shows that TCR 366 showed no alloreactivity to 103/110 MHCs tested. TCR 366-expressing pan T cells or untransduced control T cells were cocultured with MHC-null HEK293T cells re-expressing one of the 110 most frequently encountered Class I MHCs in the US population for 48 hours. A positive control consisting of HEK293T cells expressing both a fragment of PRAME which contains the 425-433 epitope (SLLQHLIGL) and HLA-A*02:01 was included in the screen. Inhibition of target cell growth by TCR 366-expressing pan T cells relative to that of untransduced control T cells was measured after 48 hours of coculture as a readout of the reactivity of the TCR 366 to allogeneic MHC molecules. The positive control and the alloreactive alleles (target cell inhibition >20%) are indicated.

FIG. 8A and FIG. 8B show the genome-wide screen identified putative off-targets for TCR 366. FIG. 8A shows an overview of the proprietary genome-wide screen. FIG. 8B shows that screen data of TCR 366 identified seven potential off-targets in a screen of >600,000 protein fragments spanning every wildtype (w.t.) human protein. The screen was designed to overpredict off-targets by overexpressing 90-aa protein fragments, which were more efficiently processed than full-length proteins, and were not physiologically recognized in healthy human primary cells (FIG. 9 below). Putative off-targets are identified by gene names.

FIG. 9A-FIG. 9D show that TCR 366 showed no reactivity to healthy human primary cells. TCR 366-expressing pan T cells or NTD cells were tested for their reactivity to primary cells derived from healthy HLA-A*02:01¹ human donors naturally expressing off-targets identified in the genome-wide safety screen. Target cells were pulsed with the PRAME₄₂₅₋₄₃₃ (SLLQHLIGL) peptide or left unpulsed, and were co-cultured with TCR 366 or NTD cells. IFN-γ secretion in culture supernatants was used as a readout of the reactivity of TCR 366 to target cells. HLA-A*02:01⁺PRAME⁺ OVCAR-3 cells were used as a positive control, and HLA-A*02:01⁺PRAME⁻ CaSki or Loucy cells were used as negative controls.

FIG. 10 provides summary data.

FIG. 11 shows pMHC dose-dependent function of processes-representative TSC-203-A0201 TCR-T Cells. T2 cells were pulsed with various concentrations of the PRAME peptide and cocultured with three batches of TSC-203-A0201 process-representative TCR-T cells. The figure shows the relative growth of T2 cells over 72 hours of co-culture with TSC-203-A0201 TCR-T cells at an E:T ratio of 2:1, normalized to t=0 h. For each donor, the co-culture was performed in triplicate (n=3). The error bars at each data point show the standard error of the mean (SEM). The area under the curve (AUC) for the resulting growth of the T2 cells over 72 hours as a function of the peptide concentration was plotted to compare the batches of TSC-203-A0201.

FIG. 12A-FIG. 12H show that TSC-203-A0201 TCR-T cells secrete Granzyme B and inflammatory cytokines IFN-γ, IL-2, TNF-α in a target-dependent manner. TSC-203-A0201 TCR-T cells (FIG. 12A-FIG. 12D) or donor matched UTF control T cells (FIG. 12E-FIG. 12H) from three donors (PD314, PD315 and PD317) were cultured in the absence of targets cells (black bars), or were cocultured at an E:T of 1:1 with either the HLA-A*A02:01 positive, PRAME-negative target cell line 647v (grey bars) or three different HLA-A*02:01-positive PRAME-positive cell lines (A375, light blue bars; Hs695T, medium blue bars; SKMEL5, dark blue bars). Supernatants were collected after 24 h co-culture and levels of inflammatory cytokines IFN-γ, IL-2 and TNF-α as well as Granzyme B were assessed with an automated 4-plex ELISA assay (ELLA from Proteinsimple). * denotes samples that were outside of the dynamic range of the assay (values are therefore less accurate), and # denotes samples for which values were beneath the detection limit.

FIG. 13A and FIG. 13B show that TSC-203-A0201 TCR-T cells proliferate in a target dependent manner. TSC-203-A0201 TCR-T cells (A) or donor matched transduced control T cells (B) from three T cell batches (PD314, PD315 and PD317) were labeled with CTV dye and were cultured in the absence of targets cells (black bars), or were cocultured at an E:T of 1:1 with either the HLA-A*A02:01 positive. PRAME-negative target cell line 647v (grey bars) or three different HLA-A*02:01-positive PRAME-positive cell lines (A375, light blue bars; Hs695T, medium blue bars; SKMEL5, dark blue bars). After 3.5 day coculture, cells were stained for flow cytometric quantification of T cell proliferation. Graphs depict the number of dividing cells (identified as CTV dim population) normalized to counting heads. The number of dividing cells is shown for the following T cell subsets: total T cells (left panels); helper T cells (middle panels) and cytotoxic T cells (right panels).

FIG. 14A and FIG. 14B show that TSC-203-A0201 TCR-T cells display potent and selective cytotoxicity. FIG. 14A shows that three batches of process-representative TSC-203-A0201 TCR-T cells (blue growth curves) and untransfected (UTF) control T cells from matched donors (gray growth curves) were analyzed in the Incucyte®-based cytotoxicity assay for their cytotoxicity potential against an HLA-A*02:01 positive, PRAME negative control cell line (647v) or three different HLA-A*02:01 positive. PRAME positive indicated target cell lines (A375, Hs695T and SKMEL5). Effector TCR-T cells and target cells were cocultured across a range of effector to target ratios (E:T ranging from 5:1 to 0.6:1) and the growth of the target cells was measured over 72 hours. Data presented were obtained with TSC-203-A0201 TCR-T cells and UTF control T cells from the batch PD315 and are representative of the data obtained with all 3 batches of process-representative material tested. Target cells cultured alone are displayed as a negative control (red growth curves). FIG. 14B shows the cytotoxic activity of the three batches of process-representative TSC-203-A0201 TCR-T cells over 72 hours which is summarized as the area under the curve (AUC) of the growth curves of target cells cocultured with TSC-203-A0201 at an E:T of 2.5:1, normalized to the growth curves of target cells cocultured with the corresponding UTF control cells.

FIG. 15A and FIG. 15B show that TSC-203-A0201 TCR-T cells are resistant to TGFβ-mediated suppression of cytokine secretion and proliferation. Three batches of process-representative TCR-T cells (PD314, PD315 and PD317) were cocultured with target cells in the presence of 0 or 5 ng/mL TGFβ1. As a control for proper TGFβ mediated T cell inhibition, two batches of process-similar TSC-203-A0201 lacking DN-TGFβRII (RG2959 164 and 6466 164) were included in the assays, as well as, in FIG. 15B, donor matched process similar TSC-203-A0201 TCR-T cells expressing DN-TGFβRII (RG2959 134 and 6466 134). FIG. 15A shows that TCR-T cells were preincubated for 24 hours with 0 or 5 ng/mL TGFβ1, and were then cocultured for 24 hours with peptide pulsed T2 cells (10 ng/mL PRAME peptide SLLQHLIGL) at an E:T of 1:1. IFN-γ secretion of TCR-T cells was evaluated after 24 hours coculture using an automated ELISA platform (ELLA from ProteinSimple). FIG. 15B shows flowcytometric evaluation of TCR-T cell proliferation after 3.5 day coculture with the HLA-A*02:01 positive and PRAME positive cancer cell line SKMEL5 (E:T 1:1). The heatmap depicts the percentage of proliferating transduced TCR-T cells observed in cocultures containing 5 ng/mL TGFβ, normalized to the percentage of proliferating TCR-T cells observed in the 0 ng/mL TGFβ condition. Proliferation data are shown for total transduced T cells (TCRαβ⁺CD34⁺), transduced helper T cells (TCRαβ⁺CD34⁺CD4⁺CD8⁺) and transduced cytotoxic T cells (TCRαβ⁺CD34⁺CD4⁺CD8⁺). Asterisks indicate process-similar control TCR-T cells that lack DN-TGFβRII.

FIG. 16 shows inoculation, dosing, and analysis schedule for animals in groups 1-7.

FIG. 17A-FIG. 17D show TSC-203-A0201 in vivo efficacy. NCG mice were inoculated subcutaneously (s.c.) with Hs 695T. Once tumor engraftment was successful (tumors reaching 100 mm³ on average, 6 days post inoculation), animals were randomized into different treatment groups. Mean tumor volume of each treatment group of mice (n=12) over time is shown FIG. 17A. Tumor volumes of individual mice over time are shown for each individual batch tested (FIG. 17B-FIG. 17D). Animals received two i.v. injections of process-representative TSC-203-A0201 TCR-T cells, or of untransfected (UTF) control T cells from matched donors, or of vehicle (PBS) on Day 1, and 8 of the study (arrow heads).

FIG. 18 shows percentage of body weight evolution over time across the different groups. NCG mice were inoculated S.C. with Hs 695T. Once tumor engraftment was successful (tumors reaching 100 mm³ on average, 6 days post inoculation), animals were randomized into different treatment groups. Animals received two i.v. injections of process-representative TSC-203-A0201 TCR-T cells (3 batches tested, PD314, 315 and PD317), or control T cells from matched donors, or of vehicle (PBS) on Day 1, and 8 of the study (arrow heads) average percentage of body weight per treatment group (n=12) is shown.

FIG. 19 shows a schematic illustrating the principle of the Target Scan screen.

FIG. 20 shows a graphical representation of results of a Target Scan screen for mechanistically representative TSC-203-A0201 TCR-T cells. Plotted is the enrichment score for each of ˜600,000 tiles/peptides in the screen calculated from 8 technical replicates, measured relative to the input. Proteins with overlapping tiles that are enriched above background are highlighted in matching colors are indicated on the graph.

FIG. 21 shows a flow chart describing the steps and timelines of the cytokine assay to test off-tumor reactivity of TSC-203-A0201 TCR-T cells.

FIG. 22 shows expression of the putative off-targets of the therapeutic TCR used in TSC-203-A0201 TCR-T cells in cancer cell lines. RNA was extracted from the cancer cell lines and sequenced. Heat maps show TPM (transcripts per million) calculated from the counts. The color scale used in RNAseq heatmaps has TPM values of zero set to white and values above zero follow a continuous color scale up to 100 TPM.

FIG. 23 shows coculture of TSC-203-A0201 TCR-T cells and UTF T cells with HLA-A*02:01*⁺ cancer cell lines expressing off-targets of the TCR. TSC-203-A0201 TCR-T cells and donor-matched UTF cells were cocultured with a panel of cancer cell lines and supernatants were evaluated for levels of IFN-γ as a measure of T cell reactivity.

FIG. 24 shows expression of putative off-targets of the therapeutic TCR used in TSC-203-A0201 TCR-T cells in primary and iPSC-derived cells. RNA was extracted from the primary and iPSC-derived cells and sequenced. Heat maps show TPM (transcripts per million) calculated from the counts. The color scale used in RNAseq heatmaps has TPM values of zero set to white and values above zero follow a continuous color scale up to 100 TPM.

FIG. 25 shows TSC-203-A0201 TCR-T cells show no reactivity to HLA-A*02:01⁺ primary cells. TSC-203-A0201 TCR-T cells and donor-matched UTF cells were cocultured with a panel of primary cells and supernatants were evaluated for levels of IFN-γ as a measure of T cell reactivity.

FIG. 26 shows steps and timelines of an oncogenicity assay to evaluate the cytokine-dependency of proliferating T cells. T cells are thawed and rested. Cells are labeled with CTV. Different media cultures are described in Table 14.

FIG. 27 shows T cell viability. Data show the normalized (using CountBright beads) numbers of viable (eFlour 660-negative) UTF and TSC-203-A0201 TCR-T cells from batch PD314, batch PD315, and batch PD317 after 5 days of in vitro culture in the absence (−) or presence (+) of cytokines and ImmunoCult™. The assay was performed in triplicate and bars show mean and standard error of the mean (SEM). The dotted line represents the initial numbers of cells (100,000) used in this assay. ****p≤0.0001; ***p≤0.001; **p≤0.01; *p≤0.05; ‘ns’ means not significant, p>0.05.

FIG. 28 shows T cell proliferation. Data show the normalized (using CountBright beads) numbers of proliferating UTF and TSC-203-A0201 TCR-T cells from batch PD314, batch PD315, and batch PD317 after 5 days of in vitro culture in the absence (−) or presence (+) of cytokines or ImmunoCult™. The assay was performed in triplicate and bars show mean and standard error of the mean (SEM). ****p≤0.0001; ***p≤0.001; **p≤0.01; *p≤0.05; ‘ns’ means not significant, p>0.05.

FIG. 29 shows percent of proliferating cells. Data show the percent (%) of proliferating UTF and TSC-203-A0201 TCR-T gated on viable cells from batch PD314, batch PD315, and batch PD317 after 5 days of culturing in the absence (−) or presence (+) of cytokines or ImmunoCult™. The assay was performed in triplicate and bars show mean and standard error of the mean (SEM). ****p≤0.0001; ***p≤0.001; **p≤0.01; *p≤0.05; ‘ns’ means not significant, p>0.05.

FIG. 30 shows PRAME expression in 48 normal human organs.

FIG. 31 shows the map of the pNVVD134_TSC-203-A02_TCR-366_MSCV-TCR-366-CD8-EF1α-dnTGFbRII-DHFR vector. Key: CD: cluster of differentiation RNA-OUT: anti-sense RNA against the bacterial levansucrase encoded by sacB. SV: simian virus TCR: T Cell Receptor, ITR: inverted terminal repeat, QBend: Mouse anti Human CD34 antibody, dnTGFbRII: Dominant-negative TGF beta Receptor II, DHFR: Dihydrofolate reductase selection marker.

FIG. 32 shows alloreactivity profiling of mechanistically representative TSC-203-A0201 TCR-T cells. Mechanistically representative TSC-203-A0201 TCR-T cells were cocultured with MHC-null HEK293T cells re-expressing one of the 110 most frequently encountered Class I HLAs in the US population for the indicated timeframe. A positive control consisting of HEK293T cells expressing both a fragment of PRAME containing the HLA-A*02:01-restricted epitope and HLA-A*02:01 (red) and a negative control consisting of MHC−/− HEK293T cells (blue) were included in the screen. The inhibition of target cell growth by the TCR-T cells relative to that by the UTD control T cells was measured over 48 h of coculture as a readout of the reactivity of mechanistically representative version of the therapeutic TCR to allogeneic HLA proteins.

FIG. 33 shows coculture of TSC-203-A0201 TCR-T cells with cancer cell lines expressing the putative allogeneic alleles. Mechanistically representative TSC-203-A0201 TCR-T cells and non-transduced (NTD) control T cells were co-cultured with cancer cell lines expressing the putative allogeneic alleles HLA-C*16:02, HLA-C*14:02, HLA-C*16:01, HLA-C*01:02, and HLA-C*08:01 for 24 h, followed by measurement of IFN-γ production in the coculture supernatant. Each cell line was also pretreated with 25 ng/mL IFN-γ, washed, and similarly co-cultured with TSC-203-A0201 TCR-T cells or NTD control T cells to examine reactivity when HLA is upregulated. PRAME-expressing HLA-A*02:01-positive Hs695T cells were included as a positive control, and PRAME-negative HLA-A*02:01-positive 647V cells were included as a negative control. The experiment was conducted with TSC-203-A0201 from two independent donors; representative data are shown.

FIG. 34 shows coculture of TSC-203-A0201 TCR-T cells with HEK293T cells overexpressing C*14:03. Mechanistically representative TSC-203-A0201 TCR-T cells or non-transduced (NTD) control T cells were co-cultured with HLA-C*14:03-overexpressing monoallelic HEK293T cells for 24 h, followed by measurement of IFN-γ production in the coculture supernatant. PRAME ORF-expressing, monoallelic A*02:01-overexpressing HEK293T cells were included as a positive control, and monoallelic A*02:01-overexpressing HEK293T cells in which PRAME had been knocked out using CRISPR/Cas9 targeting (PRAME KO HEKs) were included as a negative control. The experiment was conducted with TSC-203-A0201 from two independent donors; representative data are shown.

For any figure showing a bar histogram, curve, or other data associated with a legend, the bars, curve, or other data presented from left to right for each indication correspond directly and in order to the boxes from top to bottom, or from left to right, of the legend unless indicated otherwise.

### DETAILED DESCRIPTION OF THE INVENTION

The present invention is based, at least in part, on the discovery of PRAME immunogenic peptides (e.g., those comprising or consisting of sequences listed in Table 1), binding proteins (e.g., those having sequences listed in Table 2) that recognize PRAME antigens, and uses thereof. A systematic, comprehensive survey was carried out to map the precise T cell targets recognized by an initial pool of T cells of interest.

Accordingly, the present invention relates, in part, to the identified epitopes (immunodominant peptides) of therapeutically relevant PRAME protein and related compositions (e.g., immunodominant peptides, vaccines, and the like), compositions comprising immunogenic peptides alone or with MHC molecules, stable MHC-peptide complexes, methods of diagnosing, prognosing, and monitoring immune responses to disorders characterized by PRAME expression, and methods for preventing and/or treating disorders characterized by PRAME expression. The present invention also relates, in part, to identified binding proteins (e.g., TCRs), host cells expressing binding proteins (e.g., TCRs), compositions comprising binding proteins (e.g., TCRs) and host cells expressing binding proteins (e.g., TCRs), methods of diagnosing, prognosing, and monitoring T cell response to cells expressing PRAME, and methods for preventing and/or treating disorders characterized by PRAME expression.

### I. Definitions

For convenience, certain terms employed in the specification, examples, and appended claims are collected here.

The articles “a” and “an” are used herein to refer to one or to more than one (i.e., to at least one) of the grammatical object of the article. By way of example, “an element” means one element or more than one element. In addition, references to a table provided herein encompass all sub-tables of the table unless otherwise indicated.

The term “administering” means providing a pharmaceutical agent or composition to a subject, and includes, but is not limited to, administering by a medical professional and self-administering. This involves the physical introduction of a composition comprising a therapeutic agent to a subject, using any of the various methods and delivery systems known to those skilled in the art. In some embodiments, routes of administration for binding proteins described herein include intravenous, intraperitoneal, intramuscular, subcutaneous, spinal or other parenteral routes of administration, for example by injection or infusion. The phrase “parenteral administration” as used herein means modes of administration other than enteral and topical administration, usually by injection, and includes, without limitation, intravenous, intraperitoneal, intramuscular, intraarterial, intrathecal, intralymphatic, intralesional, intracapsular, intraorbital, intracardiac, intradermal, transtracheal, subcutaneous, subcuticular, intraarticular, subcapsular, subarachnoid, intraspinal, epidural and intrasternal injection and infusion, as well as in vivo electroporation. Alternatively, a binding protein described herein may be administered via a non-parenteral route, such as a topical, epidermal or mucosal route of administration, for example, intranasally, orally, vaginally, rectally, sublingually or topically. Administering may also be performed, for example, once, a plurality of times, and/or over one or more extended periods.

As used herein, the term “antigen” refers to any natural or synthetic immunogenic substance, such as a protein, peptide, or hapten. An antigen may be a PRAME antigen, or a fragment thereof, against which protective or therapeutic immune responses are desired. An “epitope” is the part of the antigen bound by a natural or synthetic substance.

The term “adjuvant” as used herein refers to substances, which when administered prior, together or after administration of an antigen accelerates, prolong and/or enhances the quality and/or strength of an immune response to the antigen in comparison to the administration of the antigen alone. Adjuvants can increase the magnitude and duration of the immune response induced by vaccination.

The term “antibody” as used to herein includes whole antibodies and any antigen binding fragments (i.e., “antigen-binding portions”) or single chains thereof. An “antibody” refers, in one embodiment, to a glycoprotein comprising at least two heavy (H) chains and two light (L) chains inter-connected by disulfide bonds, or an antigen binding portion thereof. Each heavy chain is comprised of a heavy chain variable region (abbreviated herein as V_{H}) and a heavy chain constant region. In certain naturally occurring antibodies, the heavy chain constant region is comprised of three domains, CH1, CH2 and CH3. In certain naturally occurring antibodies, each light chain is comprised of a light chain variable region (abbreviated herein as V_{L}) and a light chain constant region. The light chain constant region is comprised of one domain, CL. The V_{H} and V_{L} regions may be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Clq) of the classical complement system.

The term “antigen presenting cell” or “APC” includes professional antigen presenting cells (e.g., B lymphocytes, monocytes, dendritic cells, Langerhans cells), as well as other antigen presenting cells (e.g., keratinocytes, endothelial cells, astrocytes, fibroblasts, and oligodendrocytes).

The term “antigen-binding portion” of a binding protein, such as a TCR, as used herein, refers to one or more portions of a TCR that retain the ability to bind (e.g., specifically and/or selectively) to an antigen (e.g., a PRAME antigen) and cognate MHC/HLA. Such portions are, for example, between about 8 and about 1500 amino acids in length, suitably between about 8 and about 745 amino acids in length, suitably about 8 to about 300, for example about 8 to about 200 amino acids, or about 10 to about 50 or 100 amino acids in length. It has been shown that the antigen-binding function of a TCR can be performed by fragments of a full-length TCR. Examples of binding portions encompassed within the term “antigen-binding portion” of a TCR, include (i) a Fv fragment consisting of the V_{α} and V_{β} domains of a TCR, (ii) an isolated complementarity determining region (CDR) or (iii) a combination of two or more isolated CDRs which may optionally be joined by a synthetic linker. Furthermore, although V_{α} and V_{β}, are coded by separate genes, they may be joined, using recombinant methods, by a synthetic linker that enables them to be made as a single protein chain in which the V_{α} and V_{β} regions pair to form monovalent molecules (known as single chain TCR (scTCR)). Such single chain TCRs are also intended to be encompassed within the term “antigen-binding portion” of a TCR. These TCR fragments can be obtained using conventional techniques known to those with skill in the art, and the fragments are screened for utility in the same manner as are complete binding proteins. Antigen-binding portions may be produced by recombinant DNA techniques, or by enzymatic or chemical cleavage of intact immunoglobulins.

Comparator T-cell receptor” refers to at least one benchmark T-cell receptor (e.g., clone R11P3D3 or R11P3D3 KE) that has been reported in the state of the art, such as U.S. Pat. Publ, 2018/0273602. In some embodiments. “Comparator” refers to sequence R11P3D3 in U.S. Pat. Publ, 2018/0273602. In some embodiments, “Comparator Affinity Enhanced” or “Comparator AE” refers to R11P3D3_KE in U.S. Pat. Publ, 2018/0273602. Engineered versions of these parental sequences were used in the working examples and sequences of such engineered versions are set forth in Table 4. In some embodiments, the comparator T-cell receptor has sequences set forth in Table 4.

The terms “complementarity determining region” and “CDR” are synonymous with “hypervariable region” or “HVR” and are known in the art to refer to non-contiguous sequences of amino acids within certain binding proteins, such as TCR variable regions, which confer antigen specificity and/or binding affinity. For TCRs, in general, there are three CDRs in each α-chain variable region (αCDR1, αCDR2, and αCDR3) and three CDRs in each β-chain variable region (βCDR1, βCDR2, and βCDR3). CDR3 is believed to be the main CDR responsible for recognizing processed antigen. CDR1 and CDR2 mainly interact with the MHC.

The term “body fluid” refers to fluids that are excreted or secreted from the body as well as fluids that are normally not excreted or secreted from the body (e.g., amniotic fluid, aqueous humor, bile, blood and blood plasma, cerebrospinal fluid, cerumen and earwax, cowper's fluid or pre-ejaculatory fluid, chyle, chyme, stool, female ejaculate, interstitial fluid, intracellular fluid, lymph, menses, breast milk, mucus, pleural fluid, pus, saliva, sebum, semen, serum, sweat, synovial fluid, tears, urine, vaginal lubrication, vitreous humor, vomit). In some embodiments, the body fluid comprises immune cells, optionally wherein the immune cells are cytotoxic lymphocytes such as cytotoxic T cells and/or NK cells, CD4+ T cells, and the like.

The term “coding region” refers to regions of a nucleotide sequence comprising codons that are translated into amino acid residues, whereas the term “non-coding region” refers to regions of a nucleotide sequence that are not translated into amino acids (e.g., 5′ and 3′ untranslated regions).

The term “complementary” refers to the broad concept of sequence complementarity between regions of two nucleic acid strands or between two regions of the same nucleic acid strand. It is known that an adenine residue of a first nucleic acid region is capable of forming specific hydrogen bonds (“base pairing”) with a residue of a second nucleic acid region which is anti-parallel to the first region if the residue is thymine or uracil. Similarly, it is known that a cytosine residue of a first nucleic acid strand is capable of base pairing with a residue of a second nucleic acid strand which is anti-parallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. In some embodiments, the first region comprises a first portion and the second region comprises a second portion, whereby, when the first and second portions are arranged in an antiparallel fashion, at least about 50%, and, in other embodiments, at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more, or any range in between, inclusive, such as at least about 80%-100%, of the nucleotide residues of the first portion are capable of base pairing with nucleotide residues in the second portion. In some embodiments, all nucleotide residues of the first portion are capable of base pairing with nucleotide residues in the second portion.

As used herein, the term “costimulate” with reference to activated immune cells includes the ability of a costimulatory molecule to provide a second, non-activating receptor mediated signal (a “costimulatory signal”) that induces proliferation or effector function. For example, a costimulatory signal may result in cytokine secretion, e.g., in a T cell that has received a T cell-receptor-mediated signal. Immune cells that have received a cell-receptor mediated signal, e.g., via an activating receptor are referred to herein as “activated immune cells.”

“CD3” is known in the art as a multi-protein complex of six chains (see, Abbas and Lichtman, Cellular and Molecular Immunology (9^{th} Edition) (2018); Janeway et al. (Immunobiology) (9^{th} Edition) (2016)). In mammals, the complex comprises a CD3γ chain, a CD3δ chain, two CD3ε chains, and a homodimer of CD3ζ chains. The CD3γ, CD3δ, and CD3ε chains are related cell surface proteins of the immunoglobulin superfamily containing a single immunoglobulin domain. The transmembrane regions of the CD3γ, CD3δ, and CD3ε chains are negatively charged, which is a characteristic that is believed to allow these chains to associate with positively charged regions or residues of T cell receptor chains. The intracellular tails of the CD3γ, CD3δ, and CD3ε chains each contain a single conserved motif known as an immunoreceptor tyrosine-based activation motif or IT AM, whereas each CD3ζ chain has three ITAMs. Without wishing to be bound by theory, it is believed that the IT AMs are important for the signaling capacity of a TCR complex. CD3 used in accordance with the present invention may be from various animal species, including human, mouse, rat, or other mammals.

A “component of a TCR complex,” as used herein, refers to a TCR chain (i.e., TCRα, TCRβ, TCRγ or TCRδ), a CD3 chain (i.e., CD3γ, CD3δ, CD3ε or CD3), or a complex formed by two or more TCR chains or CD3 chains (e.g., a complex of TCRα and TCRβ, a complex of TCRγ and TCRδ, a complex of CD3ε and CD3δ, a complex of CD3γ and CD3ε, or a sub-TCR complex of TCRα, TCRβ, CD3γ, CD3δ, and two CD3ε chains).

“Chimeric antigen receptor” or “CAR” refers to a fusion protein that is engineered to contain two or more amino acid sequences linked together in a way that does not occur naturally or does not occur naturally in a host cell, which fusion protein can function as a receptor when present on a surface of a cell. CARs encompassed by the present invention include an extracellular portion comprising an antigen-binding domain (i.e., obtained or derived from an immunoglobulin or immunoglobulin-like molecule, such as a TCR specific for a PRAME antigen, a single chain TCR-derived binding protein, an scFv derived from an antibody, an antigen binding domain derived or obtained from a killer immunoreceptor from an NK cell, and the like) linked to a transmembrane domain and one or more intracellular signaling domains (such as an effector domain, optionally containing co-stimulatory domain(s)) (see, e.g., Sadelain et al. (2013) Cancer Discov, 3:388; see also Harris and Kranz (2016) Trends Pharmacol. Sci, 37: 220; Stone et al. (2014) Cancer Immunol. Immunother. 63:1163).

As used herein, the term “cytotoxic T lymphocyte (CTL) response” refers to an immune response induced by cytotoxic T cells. CTL responses are mediated primarily by CD8⁺ T cells.

The term “consisting essentially of” is not equivalent to “comprising” and refers to the specified materials or steps of a claim, or to those that do not materially affect the basic characteristics of a claimed subject matter. For example, a protein domain, region, or module (e.g., a binding domain, hinge region, linker module) or a protein (which may have one or more domains, regions, or modules) “consists essentially of” a particular amino acid sequence when the amino acid sequence of a domain, region, module, or protein includes extensions, deletions, mutations, or a combination thereof (e.g., amino acids at the amino- or carboxy-terminus or between domains) that, in combination, contribute to at most 20% (e.g., at most 15%, 10%, 8%, 6%, 5%, 4%, 3%, 2% or 1%) of the length of a domain, region, module, or protein and do not substantially affect (i.e., do not reduce the activity by more than 50%, such as no more than 40%, 30%, 25%, 20%, 15%, 10%, 5%, or 1%) the activity of the domain(s), region(s), module(s), or protein (e.g., the target binding affinity of a binding protein).

The term “determining a suitable treatment regimen for the subject” is taken to mean the determination of a treatment regimen (e.g., a single therapy or a combination of different therapies that are used for the prevention and/or treatment of cancer in the subject) for a subject that is started, modified and/or ended based or essentially based or at least partially based on the results of the analysis according to the present invention. One example is starting an adjuvant therapy after surgery whose purpose is to decrease the risk of recurrence, another would be to modify the dosage of a particular chemotherapy. The determination can, in addition to the results of the analysis according to the present invention, be based on personal characteristics of the subject to be treated. In most cases, the actual determination of the suitable treatment regimen for the subject will be performed by the attending physician or doctor.

The term “dominant negative TGFβ receptor” or “DN-TGFβR” refers to a transforming growth factor (TGF) beta receptor variant or mutant that provides resistance to TGFβ signaling.

There are five type II receptors (activation receptors) and seven type I receptors (signaling propagation receptors). The active TGFβ receptor is a heterotetramer consisting of two TGFβ receptors I (TGFγRI) and two TGF β receptors II (TGFβRII). In some embodiments, the DN-TGFβR is a DN-TGFβRII (i.e., a TGF beta receptor II variant or mutant). In some embodiments, resistance is to the suppressive effect of TGFβ signaling on an immune cell, such as a T cell, which TGFβ may be produced by cancer cells or by other immune cells within a cellular environment, such as by stromal cells, macrophages, myeloid cells, epithelial cells, natural killer cells, and the like. TGFβ signaling inhibitors are well-known in the art and include, without limitation, mutant TGFβ that sequesters receptors and thereby inhibits signaling, antibodies that bind to TGFβ and/or TGFβ receptors (e.g., lerdelimumab, metlimumab, fresolimumab, and the like), soluble TGFβ-binding proteins such as portions of TGFβ receptors that sequester TGFβ (e.g., TGFβRII-Fc fusion proteins) or other binders, such as beta-glycans. Any and all known TGFβ signaling inhibitors may be used instead of or in addition to DN-TGFβR (e.g., DN-TGFβRII) described herein. In some embodiments, a DN-TGFβR lacks an intracellular portion required for TGFβ-mediated signaling, such as the entire intracellular domain, a kinase signaling domain, etc. DN-TGFβR constructs are well-known in the art (see representative, non-limiting embodiments at Brand et al. (1993) J. Biol. Chem, 268:11500-11503; Weiser et al. (1993) Mol. Cell Biol. 13:7239-7247; Bollard et al. (2002) Blood 99:3179-3187; PCT Publ. WO 2009/152610; PCT Publ. WO 2017/156484; Kloss et al. (2018) Mol. Ther, 26:1855-1866; PCT Publ. WO. 2019/089884; PCT Publ. WO 2020/042647; and PCT Publ. WO 2020/042648.

As used herein, a “hematopoietic progenitor cell” is a cell that can be derived from hematopoietic stem cells or fetal tissue and is capable of further differentiation into mature cells types (e.g., immune system cells). Exemplary hematopoietic progenitor cells include those with a CD24^{Lo} Lin− CD117⁺ phenotype or those found in the thymus (referred to as progenitor thymocytes).

“Homologous” as used herein, refers to nucleotide sequence similarity between two regions of the same nucleic acid strand or between regions of two different nucleic acid strands. When a nucleotide residue position in both regions is occupied by the same nucleotide residue, then the regions are homologous at that position. A first region is homologous to a second region if at least one nucleotide residue position of each region is occupied by the same residue. Homology between two regions is expressed in terms of the proportion of nucleotide residue positions of the two regions that are occupied by the same nucleotide residue. By way of example, a region having the nucleotide sequence 5′-ATTGCC-3′ and a region having the nucleotide sequence 5′-TATGGC-3′ share 50% homology. In some embodiments, the first region comprises a first portion and the second region comprises a second portion, whereby, at least about 50%, and, in other embodiments, at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more, or any range in between, inclusive, such as at least about 80%-100%, of the nucleotide residue positions of each of the portions are occupied by the same nucleotide residue. In some embodiments, all nucleotide residue positions of each of the portions are occupied by the same nucleotide residue.

The term “hyperproliferative disorder characterized by expression of a PRAME antigen” can be any hyperproliferative disorder where the PRAME antigen is present in a MHC (e.g., HLA) complex expressed by at least some hyperproliferating cells in the subject. Examples of hyperproliferative disorders characterized by PRAME:HLA complexes include solid malignancies, such as those described in detail infra.

The term “immune response” includes T cell mediated and/or B cell mediated immune responses. Exemplary immune responses include T cell responses, e.g., cytokine production and cellular cytotoxicity. In addition, the term immune response includes immune responses that are indirectly effected by T cell activation. e.g., antibody production (humoral responses) and activation of cytokine responsive cells, e.g., macrophages.

An increased ability to stimulate an immune response or the immune system, can result from an enhanced agonist activity of T cell costimulatory receptors and/or an enhanced antagonist activity of inhibitory receptors. An increased ability to stimulate an immune response or the immune system may be reflected by a fold increase of the EC₅₀ or maximal level of activity in an assay that measures an immune response, e.g., an assay that measures changes in cytokine or chemokine release, cytolytic activity (determined directly on target cells or indirectly via detecting CD107a or granzymes) and proliferation. The ability to stimulate an immune response or the immune system activity may be enhanced by at least 10%, 20%, 30%, 40%, 50%, 60%, 70%, 80%, 90%, 100%, 110%, 120%, 130%, 140%, 150%, 160%, 170%, 180%, 190%, 200%, 250%, 300%, 350%, 400%, 500%, or more.

The term “immunotherapeutic agent” may include any molecule, peptide, antibody or other agent which can stimulate a host immune system to generate an immune response to a cancer cell in the subject. Various immunotherapeutic agents are useful in the compositions and methods described herein.

The term “immune cell” refers to any cell of the immune system that originates from a hematopoietic stem cell in the bone marrow, which gives rise to two major lineages: a myeloid progenitor cell (which give rise to myeloid cells such as monocytes, macrophages, dendritic cells, megakaryocytes and granulocytes); and a lymphoid progenitor cell (which give rise to lymphoid cells such as T cells, B cells and natural killer (NK) cells). Exemplary immune system cells include a CD4⁺ T cell, a CD8⁺ T cell, a CD4 CD8 double negative T cell, a gd T cell, a regulatory T cell, a natural killer cell, and a dendritic cell. Macrophages and dendritic cells may be referred to as “antigen presenting cells” or “APCs.” which are specialized cells that can activate T cells when a major histocompatibility complex (MHC) receptor on the surface of the APC complexed with a peptide interacts with a TCR on the surface of a T cell.

An “isolated protein” refers to a protein that is substantially free of other proteins, cellular material, separation medium, and culture medium when isolated from cells or produced by recombinant DNA techniques, or chemical precursors or other chemicals when chemically synthesized. An “isolated” or “purified” protein or biologically active portion thereof is substantially free of cellular material or other contaminating proteins from the cell or tissue source from which the binding protein, antibody, polypeptide, peptide or fusion protein is derived, or substantially free from chemical precursors or other chemicals when chemically synthesized. The language “substantially free of cellular material” includes preparations of a biomarker polypeptide or fragment thereof, in which the protein is separated from cellular components of the cells from which it is isolated or recombinantly produced. In one embodiment, the language “substantially free of cellular material” includes preparations of a biomarker protein or fragment thereof, having less than about 30% (by dry weight) of non-biomarker protein (also referred to herein as a “contaminating protein”), or, in some embodiments, less than about 25%, 20%, 15%, 10%, 5%, 1%, or less, or any range in between inclusive, such as less than about 1% to 5%, of non-biomarker protein. When binding protein, antibody, polypeptide, peptide or fusion protein or fragment thereof, e.g., a biologically active fragment thereof, is recombinantly produced, it may be substantially free of culture medium, i.e., culture medium represents less than about 20%, 15%, 10%, 5%, 1%, or less, or any range in between inclusive, such as less than about 1% to 5%, of the volume of the protein preparation.

As used herein, the term “isotype” refers to the antibody class (e.g., IgM, IgG1, IgG2C, and the like) that is encoded by heavy chain constant region genes.

As used herein, the term “Kid” is intended to refer to the dissociation equilibrium constant of a particular binding protein-antigen interaction. The binding affinity of binding proteins encompassed by the present invention may be measured or determined by standard binding protein-target binding assays, for example, competitive assays, saturation assays, or standard immunoassays, such as ELISA or RIA. A relatively lower K_{d} value indicates a relatively higher binding affinity (e.g., K_{d} values of less than or equal to about 5×10⁻⁴ M (500 uM) include a Kd value of 1×10⁻⁴ M (100 uM) and a 100 uM Kd indicates a relatively higher binding affinity as compared to a 500 uM Kd).

A “kit” is any manufacture (e.g., a package or container) comprising at least one reagent, e.g., a probe or small molecule, for specifically detecting and/or affecting the expression of a marker encompassed by the present invention. The kit may be promoted, distributed, or sold as a unit for performing the methods encompassed by the present invention. The kit may comprise one or more reagents necessary to express a composition useful in the methods encompassed by the present invention. In some embodiments, the kit may further comprise a reference standard, e.g., a nucleic acid encoding a protein that does not affect or regulate signaling pathways controlling cell growth, division, migration, survival or apoptosis. One skilled in the art can envision many such control proteins, including, but not limited to, common molecular tags (e.g., gre35 TPMen fluorescent protein and beta-galactosidase), proteins not classified in any of pathway encompassing cell growth, division, migration, survival or apoptosis by GeneOntology reference, or ubiquitous housekeeping proteins. Reagents in the kit may be provided in individual containers or as mixtures of two or more reagents in a single container. In addition, instructional materials which describe the use of the compositions within the kit may be included.

As used herein, the term “linked” refers to the association of two or more molecules. The linkage may be covalent or non-covalent. The linkage also may be genetic (i.e., recombinantly fused). Such linkages may be achieved using a wide variety of art recognized techniques, such as chemical conjugation and recombinant protein production.

A “linker,” in some embodiments, may refer to an amino acid sequence that connects two proteins, polypeptides, peptides, domains, regions, or motifs and may provide a spacer function compatible with interaction of the two sub-binding domains so that the resulting polypeptide retains a specific binding affinity (e.g., scTCR) to a target molecule or retains signaling activity (e.g., TCR complex). In some embodiments, a linker is comprised of about two to about 35 amino acids, for instance, or about four to about 20 amino acids or about eight to about 15 amino acids or about 15 to about 25 amino acids.

“Major histocompatibility complex” (MHC) refers to glycoproteins that deliver peptide antigens to a cell surface. MHC class I molecules are heterodimers having a membrane spanning a chain (with three a domains) and a non-covalently associated b2 microglobulin. MHC class II molecules are composed of two transmembrane glycoproteins, a and b, both of which span the membrane. Each chain has two domains. MHC class I molecules deliver peptides originating in the cytosol to the cell surface, where a peptide antigen-MHC (pMHC) complex is recognized by CD8⁺ T cells. MHC class II molecules deliver peptides originating in the vesicular system to the cell surface, where they are recognized by CD4⁺ T cells. Human MHC is referred to as human leukocyte antigen (HLA).

The term “PRAME” refers to PRAME nuclear receptor transcriptional regulator, an antigen that is preferentially expressed in human melanomas and that is recognized by cytolytic T lymphocytes. It is not expressed in normal tissues, except testis. The encoded protein acts as a repressor of retinoic acid receptor, and likely confers a growth advantage to cancer cells via this function. Diseases associated with PRAME include, e.g., melanoma, choroid cancer, non-small cell lung carcinomas, renal cell carcinoma (RCC), breast carcinoma, cervix carcinoma, colon carcinoma, sarcoma, neuroblastoma, head & neck cancer, ovarian cancer, as well as several types of leukemia. Human PRAME has multiple transcript variants resulted from alternative splicing, which are publicly known and can be obtained from the NCBI database. Representative human PRAME transcripts include, e.g., transcript variant 1 (NM_006115.5) encoding isoform a (NP_006106.1); transcript variant 2 (NM_206953.3) encoding isoform a (NP_996836.1); transcript variant 3 (NM_206954.3) encoding isoform a (NP_996837.1); transcript variant 4 (NM_206955.3) encoding isoform a (NP_996838.1); transcript variant 5 (NM_206956.3) encoding isoform a (NP_996839.1); transcript variant 6 (NM_001291715.2) encoding isoform a (NP_01278644.1); transcript variant 7 (NM_001291716.2) encoding isoform a (NP_001278645.1); transcript variant 8 (NM_001291717.2) encoding isoform b (NP_001278646.1); transcript variant 9 (NM_001291719.2) encoding isoform b (NP_001278648.1); transcript variant 10 (NM_001318126.2) encoding isoform b (NP_001305055.1); and transcript variant 11 (NM_001318127.2) encoding isoform b (NP_001305056.1). Representative sequences of PRAME sequences are also presented below in Table 3.

As used herein, the term “PRAME₄₂₅₋₄₃₃ antigen” or “PRAME₄₂₅₋₄₃₃ peptide antigen” or “PRAME₄₂₅₋₄₃₃-containing peptide antigen” or “PRAME₄₂₅₋₄₃₃ epitope” or “PRAME₄₂₅₋₄₃₃ peptide epitope” or “PRAME₄₂₅₋₄₃₃ bpeptide” refers to a naturally or synthetically produced peptide portion of a PRAME oncoprotein comprising, consisting of, or consisting essentially of the sequence, SLLQHLIGL.

The terms “prevent,” “preventing.” “prevention,” “prophylactic treatment,” and the like refer to reducing the probability of developing a disease, disorder, or condition in a subject, who does not have, but is at risk of or susceptible to developing a disease, disorder, or condition.

The term “prognosis” includes a prediction of the probable course and outcome of a cancer or the likelihood of recovery from the disease. In some embodiments, the use of statistical algorithms provides a prognosis of a cancer in an individual. For example, the prognosis may be surgery, development of a clinical subtype of a cancer, development of one or more clinical factors, or recovery from the disease.

As used herein, “percent identity” between amino acid sequences is synonymous with “percent homology,” which can be determined using the algorithm of Karlin and Altschul (1990) Proc. Natl. Acad. Sci. USA 87:2264-2268, modified by Karlin and Altschul (1993) Proc. Natl. Acad Sci. USA 90:5873-5877. The noted algorithm is incorporated into the NBLAST and XBLAST programs of Altschul et al. (1990) J. Mol. Biol, 215:403-410. BLAST nucleotide searches are performed with the NBLAST program, score=100, wordlength=12, to obtain nucleotide sequences homologous to a polynucleotide described herein. BLAST protein searches are performed with the XBLAST program, score=50, wordlength=3, to obtain amino acid sequences homologous to a reference polypeptide. To obtain gapped alignments for comparison purposes, Gapped BLAST is utilized as described in Altschul et al. (1997) Nuc. Acids Res, 25:3389-3402. When utilizing BLAST and Gapped BLAST programs, the default parameters of the respective programs (e.g., XBLAST and NBLAST) may be used.

The phrase “pharmaceutically-acceptable carrier” means a pharmaceutically-acceptable material, composition or vehicle, such as a liquid or solid filler, diluent, excipient, or solvent encapsulating material, involved in carrying or transporting the subject compound from one organ, or portion of the body, to another organ, or portion of the body.

The term “ratio” refers to a relationship between two numbers (e.g., scores, summations, and the like). Although, ratios may be expressed in a particular order (e.g., a to b or a:b), one of ordinary skill in the art will recognize that the underlying relationship between the numbers may be expressed in any order without losing the significance of the underlying relationship, although observation and correlation of trends based on the ratio may be reversed.

The term “recombinant host cell” (or simply “host cell”) refers to a cell that comprises a nucleic acid that is not naturally present in the cell, such as a cell into which a recombinant expression vector has been introduced. It should be understood that cells according to the present invention is intended to refer not only to the particular subject cell, but also encompasses progeny of such a cell. Because certain modifications may occur in succeeding generations due to either mutation or environmental influences, such progeny may not, in fact, be identical to the parent cell, but are still included within the scope of the term cell according to the present invention.

The term “cancer response,” “response to immunotherapy.” or “response to modulators of T-cell mediated cytotoxicity/immunotherapy combination therapy” relates to any response of the hyperproliferative disorder (e.g., cancer) to a cancer agent, such as a modulator of T-cell mediated cytotoxicity, and an immunotherapy, preferably to a change in tumor mass and/or volume after initiation of neoadjuvant or adjuvant therapy. The term “neoadjuvant therapy” refers to a treatment given before the primary treatment. Examples of neoadjuvant therapy may include chemotherapy, radiation therapy, and hormone therapy. Hyperproliferative disorder response may be assessed, for example for efficacy or in a neoadjuvant or adjuvant situation, where the size of a tumor after systemic intervention may be compared to the initial size and dimensions as measured by CT, PET, mammogram, ultrasound or palpation. Responses may also be assessed by caliper measurement or pathological examination of the tumor after biopsy or surgical resection. Response may be recorded in a quantitative fashion like percentage change in tumor volume or in a qualitative fashion like “pathological complete response” (pCR), “clinical complete remission” (cCR), “clinical partial remission” (cPR), “clinical stable disease” (cSD), “clinical progressive disease” (cPD) or other qualitative criteria. Assessment of hyperproliferative disorder response may be done early after the onset of neoadjuvant or adjuvant therapy. e.g., after a few hours, days, weeks or preferably after a few months. A typical endpoint for response assessment is upon termination of neoadjuvant chemotherapy or upon surgical removal of residual tumor cells and/or the tumor bed. This is typically three months after initiation of neoadjuvant therapy. In some embodiments, clinical efficacy of the therapeutic treatments described herein may be determined by measuring the clinical benefit rate (CBR). The clinical benefit rate is measured by determining the sum of the percentage of patients who are in complete remission (CR), the number of patients who are in partial remission (PR) and the number of patients having stable disease (SD) at a time point at least 6 months out from the end of therapy. The shorthand for this formula is CBR=CR+PR+SD over 6 months. In some embodiments, the CBR for a particular cancer therapeutic regimen is at least 25%, 30%, 35%, 40%, 45%, 50%, 55%, 60%, 65%, 70%, 75%, 80%, 85%, or more. Additional criteria for evaluating the response to cancer therapies are related to “survival,” which includes all of the following: survival until mortality, also known as overall survival (wherein said mortality may be either irrespective of cause or tumor related); “recurrence-free survival” (wherein the term recurrence shall include both localized and distant recurrence); metastasis free survival; disease free survival (wherein the term disease shall include cancer and diseases associated therewith). The length of said survival may be calculated by reference to a defined start point (e.g., time of diagnosis or start of treatment) and end point (e.g., death, recurrence or metastasis). In addition, criteria for efficacy of treatment may be expanded to include response to chemotherapy, probability of survival, probability of metastasis within a given time period, and probability of tumor recurrence. For example, in order to determine appropriate threshold values, a particular cancer therapeutic regimen may be administered to a population of subjects and the outcome may be correlated to biomarker measurements that were determined prior to administration of any cancer therapy. The outcome measurement may be pathologic response to therapy given in the neoadjuvant setting. Alternatively, outcome measures, such as overall survival and disease-free survival may be monitored over a period of time for subjects following cancer therapy for which biomarker measurement values are known. In certain embodiments, the doses administered are standard doses known in the art for cancer therapeutic agents. The period of time for which subjects are monitored may vary. For example, subjects may be monitored for at least 2, 4, 6, 8, 10, 12, 14, 16, 18, 20, 25, 30, 35, 40, 45, 50, 55, or 60 months. Biomarker measurement threshold values that correlate to outcome of a cancer therapy may be determined using well-known methods in the art, such as those described in the Examples section.

As indicated, the terms may also refer to an improved prognosis, for example, as reflected by an increased time to recurrence, which is the period to first recurrence censoring for second primary cancer as a first event or death without evidence of recurrence, or an increased overall survival, which is the period from treatment to death from any cause. To respond or to have a response means there is a beneficial endpoint attained when exposed to a stimulus. Alternatively, a negative or detrimental symptom is minimized, mitigated or attenuated on exposure to a stimulus. It will be appreciated that evaluating the likelihood that a tumor or subject will exhibit a favorable response is equivalent to evaluating the likelihood that the tumor or subject will not exhibit favorable response (i.e., will exhibit a lack of response or be non-responsive).

The term “resistance” refers to an acquired or natural resistance of a cancer sample or a mammal to a cancer therapy (i.e., being nonresponsive to or having reduced or limited response to the therapeutic treatment), such as having a reduced response to a therapeutic treatment by 5%, 10%, 15%, 20%, 25%, 30%, 35%, 40%, 45%, 50%, 55%, 60%, 65%, 70%, 75%, 80%, 85%, 90%, 95%, 100%, or more, such 2-fold, 3-fold, 4-fold, 5-fold, 10-fold, 15-fold, 20-fold or more, or any range in between, inclusive. The reduction in response may be measured by comparing with the same cancer sample or mammal before the resistance is acquired, or by comparing with a different cancer sample or a mammal that is known to have no resistance to the therapeutic treatment. A typical acquired resistance to chemotherapy is called “multidrug resistance.” The multidrug resistance may be mediated by P-glycoprotein or may be mediated by other mechanisms, or it may occur when a mammal is infected with a multi-drug-resistant microorganism or a combination of microorganisms. The determination of resistance to a therapeutic treatment is routine in the art and within the skill of an ordinarily skilled clinician, for example, may be measured by cell proliferative assays and cell death assays as described herein as “sensitizing.” In some embodiments, the term “reverses resistance” means that the use of a second agent in combination with a primary cancer therapy (e.g., chemotherapeutic or radiation therapy) is able to produce a significant decrease in tumor volume at a level of statistical significance (e.g., p<0.05) when compared to tumor volume of untreated tumor in the circumstance where the primary cancer therapy (e.g., chemotherapeutic or radiation therapy) alone is unable to produce a statistically significant decrease in tumor volume compared to tumor volume of untreated tumor. This generally applies to tumor volume measurements made at a time when the untreated tumor is growing logarithmically.

The term “sample” used for detecting or determining the absence, presence, or level of at least one biomarker is typically brain tissue, cerebrospinal fluid, whole blood, plasma, serum, saliva, urine, stool (e.g., feces), tears, and any other bodily fluid (e.g., as described above under the definition of “body fluids”), or a tissue sample (e.g., biopsy) such as a small intestine, colon sample, or surgical resection tissue. In some embodiments, methods encompassed by the present invention further comprises obtaining the sample from the individual prior to detecting or determining the absence, presence, or level of at least one marker in the sample.

The term “sensitize” means to alter cancer cells or tumor cells in a way that allows for more effective treatment of the associated cancer with a cancer therapy (e.g., anti-immune checkpoint, chemotherapeutic, and/or radiation therapy). In some embodiments, normal cells are not affected to an extent that causes the normal cells to be unduly injured by the therapies. An increased sensitivity or a reduced sensitivity to a therapeutic treatment is measured according to a known method in the art for the particular treatment and methods described herein below, including, but not limited to, cell proliferative assays (Tanigawa et al. (1982) Cancer Res, 42:2159-2164) and cell death assays (Weisenthal et al. (1984) Cancer Res. 94:161-173; Weisenthal et al. (1985) Cancer Treat Rep, 69:615-632; Weisenthal et al., In: Kaspers G J L, Pieters R, Twentyman P R, Weisenthal L M, Veerman A J P, eds. Drug Resistance in Leukemia and Lymphoma. Langhorne. P A: Harwood Academic Publishers, 1993:415-432; Weisenthal (1994) Contrib. Gynecol. Obstet, 19:82-90). The sensitivity or resistance may also be measured in animal by measuring the tumor size reduction over a period of time, for example, 6 month for human and 4-6 weeks for mouse. A composition or a method sensitizes response to a therapeutic treatment if the increase in treatment sensitivity or the reduction in resistance is 5%, 10%, 15%, 20%, 25%, 30%, 35%, 40%, 45%, 50%, 55%, 60%, 65%, 70%, 75%, 80%, 85%, 90%, 95%, 100%, or more, such 2-fold, 3-fold, 4-fold, 5-fold, 10-fold, 15-fold, 20-fold or more, or any range in between, inclusive, compared to treatment sensitivity or resistance in the absence of such composition or method. The determination of sensitivity or resistance to a therapeutic treatment is routine in the art and within the skill of an ordinarily skilled clinician. It is to be understood that any method described herein for enhancing the efficacy of a cancer therapy may be equally applied to methods for sensitizing hyperproliferative or otherwise cancerous cells (e.g., resistant cells) to the cancer therapy.

The term “small molecule” is a term of the art and includes molecules that are less than about 1000 molecular weight or less than about 500 molecular weight. In one embodiment, small molecules do not exclusively comprise peptide bonds. In another embodiment, small molecules are not oligomeric. Exemplary small molecule compounds which may be screened for activity include, but are not limited to, peptides, peptidomimetics, nucleic acids, carbohydrates, small organic molecules (e.g., polyketides) (Cane et al. (1998) Science 282:63-68), and natural product extract libraries. In another embodiment, the compounds are small, organic non-peptidic compounds. In a further embodiment, a small molecule is not biosynthetic.

The term “specific binding” refers to binding protein binding to a predetermined antigen. Typically, the binding protein binds with an affinity (Ku) of approximately less than or equal to about 5×10⁻⁴ M, less than or equal to about 1×10⁻⁴ M, less than or equal to about 5×10⁻⁵ M, less than or equal to about 1×10⁻⁵ M, less than or equal to about 5×10⁻⁶ M, less than or equal to about 1×10⁻⁶ M, less than or equal to about 5×10⁻⁷ M, less than or equal to about 1×10⁻⁷ M, less than or equal to about 5×10⁻⁸ M, less than or equal to about 1×10⁻⁸ M, less than or equal to about 5×10⁻⁹ M, less than or equal to about 1×10⁻⁹ M, less than or equal to about 5×10⁻¹⁰ M, less than or equal to about 1×10⁻¹⁰ M, less than or equal to about 5×10⁻¹¹ M, less than or equal to about 1×10⁻¹¹ M, less than or equal to about 5×10⁻¹² M, less than or equal to about 1×10⁻¹² M, or even lower, or any range in between, inclusive, such as between about 1-50 micromolar, 1-100 micromolar, 0.1-500 micromolar, and the like, when determined by a binding assay, such as surface plasmon resonance (SPR) technology in a BIAcore™ assay instrument using an antigen of interest as the analyte and the binding protein as the ligand. In some embodiments, the binding protein binds to the predetermined antigen with an affinity that is at least 1.1-, 1.2-, 1.3-, 1.4-, 1.5-, 1.6-, 1.7-, 1.8-, 1.9-, 2.0-, 2.5-, 3.0-, 3.5-, 4.0-, 4.5-, 5.0-, 6.0-, 7.0-, 8.0-, 9.0-, or 10.0-fold or greater than its affinity for binding to a non-specific antigen (e.g., BSA, casein) other than the predetermined antigen or a closely-related antigen. The phrases “a binding protein recognizing an antigen” and “a binding protein specific for an antigen” are used interchangeably herein with the term “a binding protein which binds specifically to an antigen.” Selective binding is a relative term referring to the ability of a binding protein to discriminate the binding of one antigen over another, such as a particular family member or antigen target over a related family member or antigen target. For example, analytical data provided in the Examples section demonstrate that binding proteins described herein specifically bind PRAME immunogenic epitopes and/or selectively bind a number of related epitopes (e.g., PRAME immunogenic epitopes and closely related sequences) discriminating such targets from the vast majority of other possible epitopes available in the human genome.

The term “subject” refers to any healthy animal, mammal or human, or any animal, mammal or human afflicted with a disorder characterized by PRAME expression, such as a non-malignant disorder, a hyperproliferative disorder, or a relapse of a hyperproliferative disorder characterized by PRAME expression. The term “subject” is interchangeable with “patient.”

The term “survival” includes all of the following: survival until mortality, also known as overall survival (wherein said mortality may be either irrespective of cause or tumor related); “recurrence-free survival” (wherein the term recurrence shall include both localized and distant recurrence); metastasis free survival: disease free survival (wherein the term disease shall include cancer and diseases associated therewith). The length of said survival may be calculated by reference to a defined start point (e.g., time of diagnosis or start of treatment) and end point (e.g., death, recurrence or metastasis). In addition, criteria for efficacy of treatment may be expanded to include response to chemotherapy, probability of survival, probability of metastasis within a given time period, and probability of tumor recurrence.

The term “synergistic effect” refers to the combined effect of two or more agents (e.g., a PRAME-related agent described herein and another therapy, such as an additional PRAME-targeted TCR, anti-cancer therapy, immunotherapy, etc. for treating a disorder characterized by PRAME expression) that is greater than the sum of the separate effects of the cancer agents/therapies alone.

As used herein, the term “T cell-mediated response” refers to a response mediated by T cells, including effector T cells (e.g., CD8⁺ cells) and helper T cells (e.g., CD4⁺ cells). T cell mediated responses include, for example, T cell cytotoxicity and proliferation.

A “transcribed polynucleotide” or “nucleotide transcript” is a polynucleotide (e.g., an mRNA, hnRNA, a cDNA, or an analog of such RNA or cDNA) which is complementary to or homologous with all or a portion of a mature mRNA made by transcription of a biomarker nucleic acid and normal post-transcriptional processing (e.g., splicing), if any, of the RNA transcript, and reverse transcription of the RNA transcript.

A “T cell” is an immune system cell that matures in the thymus and produces T cell receptors (TCRs). T cells may be naive (not exposed to antigen: increased expression of CD62L, CCR7, CD28, CD3, CD 127, and CD45RA, and decreased expression of CD45RO as compared to T_{CM}), memory T cells (T_{M}) (antigen-experienced and long-lived), and effector cells (antigen-experienced, cytotoxic). T_{M} may be further divided into subsets of central memory T cells (T_{CM}, increased expression of CD62L, CCR7, CD28, CD127, CD45RO, and CD95, and decreased expression of CD54RA as compared to naive T cells) and effector memory T cells (T_{EM}, decreased expression of CD62L, CCR7, CD28, CD45RA, and increased expression of CD127 as compared to naive T cells or T_{CM}). Effector T cells (T_{E}) refers to antigen-experienced CD8+ cytotoxic T lymphocytes that have decreased expression of CD62L, CCR7, CD28, and are positive for granzyme and perforin as compared to T_{CM}. Other exemplary T cells include regulatory T cells, such as CD4⁺CD25⁺ (Foxp3⁺) regulatory T cells and Tregl7 cells, as well as Trl, Th3, CD8⁺CD28, and Qa-1 restricted T cells.

Conventional T cells, also known as Tconv or Teffs, have effector functions (e.g., cytokine secretion, cytotoxic activity, anti-self-recognition, and the like) to increase immune responses by virtue of their expression of one or more T cell receptors. Tcons or Teffs are generally defined as any T cell population that is not a Treg and include, for example, naïve T cells, activated T cells, memory T cells, resting Tcons, or Tcons that have differentiated toward, for example, the Th1 or Th2 lineages. In some embodiments, Teffs are a subset of non-Treg T cells. In some embodiments, Teffs are CD4+ Teffs or CD8+ Teffs, such as CD4+ helper T lymphocytes (e.g., Th0, Th1, Tfh, or Th17) and CD8+ cytotoxic T lymphocytes. As described further herein, cytotoxic T cells are CD8+ T lymphocytes. “Naïve Tcons” are CD4⁺ T cells that have differentiated in bone marrow, and successfully underwent a positive and negative processes of central selection in a thymus, but have not yet been activated by exposure to an antigen. Naïve Tcons are commonly characterized by surface expression of L-selectin (CD62L), absence of activation markers such as CD25, CD44 or CD69, and absence of memory markers such as CD45RO. Naïve Tcons are therefore believed to be quiescent and non-dividing, requiring interleukin-7 (IL-7) and interleukin-15 (IL-15) for homeostatic survival (see, at least WO 2010/101870). The presence and activity of such cells are undesired in the context of suppressing immune responses. Unlike Tregs, Tcons are not anergic and can proliferate in response to antigen-based T cell receptor activation (Lechler et al. (2001) Philos. Trans. R. Soc. Lond. Biol. Sci. 356:625-637).

“T effector” (“T_{eff}” or “T_{E}”) cells refers to T cells (e.g., CD4+ and CD8+ T cells) with cytolytic activities as well as T helper (Th) cells, which secrete cytokines and activate and direct other immune cells, but does not include regulatory T cells (Treg cells).

“T cell receptor” or “TCR” refers to an immunoglobulin superfamily member (having a variable binding domain, a constant domain, a transmembrane region, and a short cytoplasmic tail; see, e.g., Janeway et al. (1997) Curr. Biol. Publ, 4:33) that is capable of binding (e.g., specifically and/or selectively) to an antigen peptide bound to a MHC receptor. A TCR can be found on the surface of a cell or in soluble form and generally is comprised of a heterodimer having alpha and beta chains (also known as TCRα and TCRβ, respectively), or γ and δ chains (also known as TCRγ and TCRδ, respectively). Like immunoglobulins (e.g., antibodies), the extracellular portion of TCR chains (e.g., α-chain and β-chain) contain two immunoglobulin domains: a variable domain (e.g., α-chain variable domain or V_{α} and β-chain variable domain or V_{β}; typically amino acids 1 to 116 based on Kabat numbering (Kabat et al. (1991) “Sequences of Proteins of Immunological Interest, US Dept. Health and Human Services. Public Health Service National Institutes of Health, 5^{th} ed.) at the N-terminal end, and one constant domain (e.g., α-chain constant domain or C_{α}, typically amino acids 117 to 259 based on Kabat, β-chain constant domain or C_{β}, typically amino acids 117 to 295 based on Kabat) at the C-terminal end and adjacent to the cell membrane. Also like immunoglobulins, the variable domains contain complementary determining regions (“CDRs”, also called hypervariable regions or “HVRs”) separated by framework regions (“FRs”) (see, e.g., Fores et al. (1990) Proc. Nat. Acad Sci. USA, 87:9138; Chothia et al. (1988) EMBO J, 7:3745; Lefranc et al. (2003) Dev. Comp. Immunol, 27:55). In some embodiments, a TCR is found on the surface of a T cell (or T lymphocyte) and associates with the CD3 complex. The source of a TCR encompassed by the present invention may be from various animal species, such as a human, mouse, rat, rabbit or other mammal.

The term “T cell receptor” or “TCR” should be understood to encompass full TCRs as well as antigen-binding portions or antigen-binding fragments thereof. In some embodiments, the TCR is an intact or full-length TCR, including TCRs in the αβ form or γδ form. In some embodiments, the TCR is an antigen-binding portion that is less than a full-length TCR but that binds to a specific peptide bound in an MHC molecule, such as binds to an MHC-peptide complex. In some cases, an antigen-binding portion or fragment of a TCR may contain only a portion of the structural domains of a full-length or intact TCR, but yet is able to bind the peptide epitope, such as MHC-peptide complex, to which the full TCR binds. In some cases, an antigen-binding portion contains the variable domains of a TCR, such as variable α chain and variable β chain of a TCR, sufficient to form a binding site for binding to a specific MHC-peptide complex. Generally, the variable chains of a TCR contain complementarity determining regions (CDRs) involved in recognition of the peptide, MHC and/or MHC-peptide complex.

Nomenclature established by the International Immunogenetics Information System (IMGT) (see also Scaviner and Lefranc (2000) Exp. Clin. Immunogenet, 17:83-96 and 97-106; Folch and Lefranc (2000) Exp. Clin. Immunogenet, 17:107-114; T Cell Receptor Factsbook”, (2001) LeFranc and LeFranc, Academic Press. ISBN 0-12-441352-8). The IMGT provides unique sequences used to describe a TCR, and sequences described herein may be identified by reference to such unique sequences provided herein. TCR sequences are publicly available at the IMGT database at imgt.org.

As described above, native alpha/beta heterodimeric TCRs have an alpha chain and a beta chain. Broadly, each chain comprises variable, joining and constant regions, and the beta chain also usually contains a short diversity region between the variable and joining regions, but this diversity region is often considered as part of the joining region. Each variable region comprises three hypervariable CDRs (Complementarity Determining Regions) embedded in a framework sequence. CDR3 is well-known to be the main mediator of antigen recognition. There are several types of alpha chain variable (V_{α}) regions and several types of beta chain variable (Vβ) regions distinguished by their framework, CDR1 and CDR2 sequences, and by a partly defined CDR3 sequence. The V_{α} types are referred to in IMGT nomenclature by a unique TRAV number. For example, “TRAV4” defines a TCR V_{α} region having unique framework and CDR1 and CDR2 sequences, and a CDR3 sequence which is partly defined by an amino acid sequence which is preserved from TCR to TCR but which also includes an amino acid sequence which varies from TCR to TCR. Similarly, “TRBV2” defines a TCR Vβ region having unique framework and CDR1 and CDR2 sequences, but with only a partly defined CDR3 sequence. It is known that there are 54 alpha variable genes, of which 44 are functional, and 67 beta variable genes, of which 42 are functional, within the alpha and beta loci, respectively.

The joining regions of the TCR are similarly defined by the unique IMGT TRAJ and TRBJ nomenclature, and the constant regions by the IMGT TRAC and TRBC nomenclature. The beta chain diversity region is referred to in IMGT nomenclature by the abbreviation TRBD, and, as mentioned, the concatenated TRBD/TRBJ regions are often considered together as the joining region.

The gene pools that encode the TCR alpha and beta chains are located on different chromosomes and contain separate V, (D), J and C gene segments, which are brought together by rearrangement during T cell development. This leads to a very high diversity of T cell alpha and beta chains due to the large number of potential recombination events that occur between the 54 TCR alpha variable genes and 61 alpha J genes or between the 67 beta variable genes, two beta D genes and 13 beta J genes. The recombination process is not precise and introduces further diversity within the CDR3 region. Each alpha and beta variable gene may also comprise allelic variants, designated in IMGT nomenclature as TRAVxx*01 and *02, or TRBVx-x*01 and *02 respectively, thus further increasing the amount of variation. In the same way, some of the TRBJ sequences have two known variations. (Note that the absence of a “*” qualifier means that only one allele is known for the relevant sequence). The natural repertoire of human TCRs resulting from recombination and thymic selection has been estimated to comprise approximately 10⁶ unique beta chain sequences, determined from CDR3 diversity (Arstila et al. (1999) Science 286:958-961) and could be even higher (Robins et al. (2009) Blood 114:4099-4107). Each beta chain is estimated to pair with at least 25 different alpha chains, thus generating further diversity (Arstila et al. (1999) Science 286:958-961).

The term “TCR alpha variable domain” therefore refers to the concatenation of TRAV and TRAJ regions; a TRAV region only; or TRAV and a partial TRAJ region, and the term TCR alpha constant domain refers to the extracellular TRAC region, or to a C-terminal truncated or full length TRAC sequence. Likewise the term “TCR beta variable domain” refers to the concatenation of TRBV and TRBD/TRBJ regions; to the TRBV and TRBD regions only; to the TRBV and TRBJ regions only; or to the TRBV and partial TRBD and/or TRBJ regions, and the term TCR beta constant domain refers to the extracellular TRBC region, or to a C-terminal truncated or full length TRBC sequence. These TCR alpha variable domain and TCR beta variable domain nomenclature similarly applies to the variable domains of TCR gamma and TCR delta chains, respectively, for gamma/delta TCRs. An ordinarily skilled artisan can obtain TRAV. TRAJ, TRAC, TRBV, TRBJ, and TRBC gene sequences, such as through the publicly available IMGT database.

The term “TCR complex” refers to a complex formed by the association of CD3 with TCR. For example, a TCR complex may be composed of a CD3γ chain, a CD3δ chain, two CD3ε chains, a homodimer of CD3ζ chains, a TCRα chain, and a TCRδ chain. Alternatively, a TCR complex may be composed of a CD3γ chain, a CD3δ chain, two CD3ε chains, a homodimer of CD3ζ chains, a TCRγ chain, and a TCRS chain.

The term “therapeutic effect” refers to a local or systemic effect in animals, particularly mammals, and more particularly humans, caused by a pharmacologically active substance. The term thus means any substance intended for use in the diagnosis, cure, mitigation, treatment or prevention of disease or in the enhancement of desirable physical or mental development and conditions in an animal or human.

The terms “therapeutically effective amount” and “effective amount” means that amount of a substance that produces some desired effect, such as a desired local or systemic therapeutic effect, in at least a sub-population of cells in an animal at a reasonable benefit/risk ratio applicable to any treatment. In some embodiments, a therapeutically effective amount of a substance will depend on the substance's therapeutic index, solubility, pharmacokinetics, half-life, and the like. Toxicity and therapeutic efficacy of subject compounds may be determined by standard pharmaceutical procedures in cell cultures or experimental animals, e.g., for determining the LD₅₀ and the ED₅₀. In some embodiments, compositions that exhibit large therapeutic indices are used. In some embodiments, the LD₅₀ (lethal dosage) may be measured and may be, for example, at least 10%, 20%, 30%, 40%, 50%, 60%, 70%, 80%, 90%, 100%, 200%, 300%, 400%, 500%, 600%, 700%, 800%, 900%, 1000% or more reduced for the agent relative to no administration of the agent. Similarly, the ED₅₀ (i.e., the concentration which achieves a half-maximal inhibition of symptoms) may be measured and may be, for example, at least 10%, 20%, 30%, 40%, 50%, 60%, 70%, 80%, 90%, 100%, 200%, 300%, 400%, 500%, 600%, 700%, 800%, 900%, 1000% or more increased for the agent relative to no administration of the agent. Also, similarly, the IC₅₀ may be measured and may be, for example, at least 10%, 20%, 30%, 40%, 50%, 60%, 70%, 80%, 90%, 100%, 200%, 300%, 400%, 500%, 600%, 700%, 800%, 900%, 1000% or more increased for the agent relative to no administration of the agent. In some embodiments. T cell immune response in an assay may be increased by at least about 10%, 15%, 20%, 25%, 30%, 35%, 40%, 45%, 50%, 55%, 60%, 65%, 70%, 75%, 80%, 85%, 90%, 95%, or even 100%. In another embodiment, at least about a 10%, 15%, 20%, 25%, 30%, 35%, 40%, 45%, 50%, 55%, 60%, 65%, 70%, 75%, 80%, 85%, 90%, 95%, or even 100% decrease in a viral load may be achieved.

The term “treat” refers to the therapeutic management or improvement of a condition (e.g., a disease or disorder) of interest. Treatment may include, but is not limited to, administering an agent or composition (e.g., a pharmaceutical composition) to a subject. Treatment is typically undertaken in an effort to alter the course of a disease (which term is used to indicate any disease, disorder, syndrome or undesirable condition warranting or potentially warranting therapy) in a manner beneficial to the subject. The effect of treatment may include reversing, alleviating, reducing severity of, delaying the onset of, curing, inhibiting the progression of, and/or reducing the likelihood of occurrence or recurrence of the disease or one or more symptoms or manifestations of the disease. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. A therapeutic agent may be administered to a subject who has a disease or is at increased risk of developing a disease relative to a member of the general population. In some embodiments, a therapeutic agent may be administered to a subject who has had a disease but no longer shows evidence of the disease. The agent may be administered e.g., to reduce the likelihood of recurrence of evident disease. A therapeutic agent may be administered prophylactically, i.e., before development of any symptom or manifestation of a disease. “Prophylactic treatment” refers to providing medical and/or surgical management to a subject who has not developed a disease or does not show evidence of a disease in order, e.g., to reduce the likelihood that the disease will occur or to reduce the severity of the disease should it occur. The subject may have been identified as being at risk of developing the disease (e.g., at increased risk relative to the general population or as having a risk factor that increases the likelihood of developing the disease.

The term “unresponsiveness” includes refractivity of cancer cells to therapy or refractivity of therapeutic cells, such as immune cells, to stimulation, e.g., stimulation via an activating receptor or a cytokine. Unresponsiveness may occur, e.g., because of exposure to immunosuppressants or exposure to high doses of antigen. As used herein, the term “anergy” or “tolerance” includes refractivity to activating receptor-mediated stimulation. Such refractivity is generally antigen-specific and persists after exposure to the tolerizing antigen has ceased. For example, anergy in T cells (as opposed to unresponsiveness) is characterized by lack of cytokine production. e.g., IL-2. T cell anergy occurs when T cells are exposed to antigen and receive a first signal (a T cell receptor or CD-3 mediated signal) in the absence of a second signal (a costimulatory signal). Under these conditions, reexposure of the cells to the same antigen (even if reexposure occurs in the presence of a costimulatory polypeptide) results in failure to produce cytokines and, thus, failure to proliferate. Anergic T cells may, however, proliferate if cultured with cytokines (e.g., IL-2). For example, T cell anergy may also be observed by the lack of IL-2 production by T lymphocytes as measured by ELISA or by a proliferation assay using an indicator cell line. Alternatively, a reporter gene construct may be used. For example, anergic T cells fail to initiate IL-2 gene transcription induced by a heterologous promoter under the control of the 5′ IL-2 gene enhancer or by a multimer of the API sequence that may be found within the enhancer (Kang et al. (1992) Science 257:1134).

The term “vaccine” refers to a pharmaceutical composition that elicits an immune response to an antigen of interest. The vaccine may also confer protective immunity upon a subject.

The term “variable region” or “variable domain” refers to the domain of an immunoglobulin superfamily binding protein (e.g., a TCR α-chain or β-chain (or γ chain and δ chain for γδ TCRs)) that is involved in binding of the immunoglobulin superfamily binding protein (e.g., TCR) to antigen. The variable domains of the α-chain and β-chain (V_{α} and V_{β}, respectively) of a native TCR generally have similar structures, with each domain comprising four conserved framework regions (FRs) and three CDRs. The V_{α} domain is encoded by two separate DNA segments, the variable gene segment and the joining gene segment (V-J); the V_{β} domain is encoded by three separate DNA segments, the variable gene segment, the diversity gene segment, and the joining gene segment (V-D-J). A single V_{α} or VP domain may be sufficient to confer antigen-binding specificity. Furthermore, TCRs that bind a particular antigen may be isolated using a V_{α} or V_{β} domain from a TCR that binds the antigen to screen a library of complementary V_{α} or V_{β} domains, respectively.

The term “vector” refers to a nucleic acid molecule capable of transporting another nucleic acid to which it has been linked. In some embodiments, a vector is an episome, i.e., a nucleic acid capable of extra-chromosomal replication. In some embodiments, vectors are those capable of autonomous replication and/or expression of nucleic acids to which they are linked. Vectors capable of directing the expression of genes to which they are operatively linked are referred to herein as “expression vectors”. In general, expression vectors of utility in recombinant DNA techniques are often in the form of “plasmids” which refer generally to circular double stranded DNA loops, which, in their vector form are not bound to the chromosome. In the present specification. “plasmid” and “vector” are used interchangeably as the plasmid is the most commonly used form of vector. However, as will be appreciated by those skilled in the art, the present invention is intended to include such other forms of expression vectors that serve equivalent functions and which become subsequently known in the art.

There is a known and definite correspondence between the amino acid sequence of a particular protein and the nucleotide sequences that can code for the protein, as defined by the genetic code (shown below). Likewise, there is a known and definite correspondence between the nucleotide sequence of a particular nucleic acid and the amino acid sequence encoded by that nucleic acid, as defined by the genetic code.

GENETIC CODE

| | |
|---|---|
| Alanine (Ala, A) | GCA, GCC, GCG, GCT |
| Arginine (Arg, R) | AGA, ACG, CGA, CGC, CGG, CGT |
| Asparagine (Asn, N) | AAC, AAT |
| Aspartic acid (Asp, D) | GAC, GAT |
| Cysteine (Cys, C) | TGC, TGT |
| Glutamic acid (Glu, E) | GAA, GAG |
| Glutamine (Gln, Q) | CAA, CAG |
| Glycine (Gly, G) | GGA, GGC, GGG, GGT |
| Histidine (His, H) | CAC, CAT |
| Isoleucine (Ile, I) | ATA, ATC, ATT |
| Leucine (Leu, L) | CTA, CTC, CTG, CTT, TTA, TTG |
| Lysine (Lys, K) | AAA, AAG |
| Methionine (Met, M) | ATG |
| Phenylalanine (Phe, F) | TTC, TTT |
| Proline (Pro, P) | CCA, CCC, CCG, CCT |
| Serine (Ser, S) | AGC, AGT, TCA, TCC, TCG, TCT |
| Threonine (Thr, T) | ACA, ACC, ACG, ACT |
| Tryptophan (Trp, W) | TGG |
| Tyrosine (Tyr, Y) | TAC, TAT |
| Valine (Val, V) | GTA, GTC, GTG, GTT |
| Termination signal (end) | TAA, TAG, TGA |

An important and well-known feature of the genetic code is its redundancy, whereby, for most of the amino acids used to make proteins, more than one coding nucleotide triplet may be employed (illustrated above). Therefore, a number of different nucleotide sequences may code for a given amino acid sequence. Such nucleotide sequences are considered functionally equivalent since they result in the production of the same amino acid sequence in all organisms (although certain organisms may translate some sequences more efficiently than they do others). Moreover, occasionally, a methylated variant of a purine or pyrimidine may be found in a given nucleotide sequence. Such methylations do not affect the coding relationship between the trinucleotide codon and the corresponding amino acid.

In view of the foregoing, the nucleotide sequence of a DNA or RNA encoding a biomarker nucleic acid (or any portion thereof) may be used to derive the polypeptide amino acid sequence, using the genetic code to translate the DNA or RNA into an amino acid sequence. Likewise, for polypeptide amino acid sequence, corresponding nucleotide sequences that can encode the polypeptide can be deduced from the genetic code (which, because of its redundancy, will produce multiple nucleic acid sequences for any given amino acid sequence). Thus, description and/or disclosure herein of a nucleotide sequence which encodes a polypeptide should be considered to also include description and/or disclosure of the amino acid sequence encoded by the nucleotide sequence. Similarly, description and/or disclosure of a polypeptide amino acid sequence herein should be considered to also include description and/or disclosure of all possible nucleotide sequences that can encode the amino acid sequence.

### II. Peptides

In certain aspects, provided herein are methods and compositions for the treatment and/or prevention of disorders associated with PRAME expression through the induction of an immune response against PRAME or cells expressing PRAME relating to administration of PRAME immunogenic peptides, nucleic acids encoding same, and/or cells expressing same, described herein.

In certain embodiments, the PRAME immunogenic peptide comprises (e.g., consists of) a peptide epitope selected from peptide sequences listed in Table 1, such as Table 1A. Peptide epitopes described herein may be combined with MHC molecules, such as particular HLA molecules having particular HLA alpha chain alleles. For example, Table 1A peptides were identified in association with an MHC whose alpha chain had an HLA-A*02 serotype, such as that encoded by an HLA-A*02:01 allele, as described further in the Examples section. In some embodiments, PRAME immunogenic peptides may be combined with an MHC molecule, wherein the MHC molecule comprises an MHC alpha chain that is an HLA serotype selected from the group consisting of HLA-A*02, HLA-A*03, HLA-A*01, HLA-A*11, HLA-A*24, HLA-B*07, HLA-C*07, HLA-C*01, HLA-C*02, HLA-C*03, HLA-C*04, HLA-C*05, HLA-C*06, HLA-C*08, HLA-C*12, HLA-C*14, HLA-C*15, HLA-C*16, HLA-C*17, and HLA-C*18, optionally wherein the HLA allele is selected from the group consisting of HLA-A*02:01, HLA-A*02:02, HLA-A*02:03, HLA-A*02:04, HLA-A*02:05, HLA-A*02:06, HLA-A*02:07, HLA-A*02:10, HLA-A*02:11, HLA-A*02:12, HLA-A*02:13, HLA-A*02:14, HLA-A*02:16, HLA-A*02:17, HLA-A*02:19, HLA-A*02:20, HLA-A*02:22, HLA-A*02:24, HLA-A*02:30, HLA-A*02:42, HLA-A*02:53, HLA-A*02:60, HLA-A*02:74 allele, HLA-A*03:01, HLA-A*03:02, HLA-A*03:05, HLA-A*03:07, HLA-A*01:01, HLA-A*01:02, HLA-A*01:03, HLA-A*01:16 allele, HLA-A*11:01, HLA-A*11:02, HLA-A*11:03, HLA-A*11:04, HLA-A*11:05, HLA-A*11:19 allele, HLA-A*24:02, HLA-A*24:03, HLA-A*24:05, HLA-A*24:07, HLA-A*24:08, HLA-A*24:10, HLA-A*24:14, HLA-A*24:17, HLA-A*24:20, HLA-A*24:22, HLA-A*24:25, HLA-A*24:26, HLA-A*24:58 allele, HLA-B*07:02, HLA-B*07:04, HLA-B*07:05, HLA-B*07:09, HLA-B*07:10, HLA-B*07:15, HLA-B*07:21, HLA-C*07:02, HLA-C*07:01, HLA-C*04:01, HLA-C*06:02, HLA-C*03:04, HLA-C*05:01, HLA-C*16:01, HLA-C*02:02, HLA-C*03:03, HLA-C*12:03, HLA-C*08:02, HLA-C*01:02, HLA-C*17:01, HLA-C*15:02, HLA-C*14:02, HLA-C*12:02, HLA-C*07:04, HLA-C*08:01, HLA-C*03:02, HLA-C*18:01, HLA-C*15:05, HLA-C*16:02, HLA-C*08:04, HLA-C*03:05, and HLA-C*14:03 allele. In some embodiments, the PRAME immunogenic peptides are derived from a human PRAME protein and/or a PRAME protein shown in Table 3. In some embodiments, one or more PRAME immunogenic peptides are administered alone or in combination with an adjuvant.

In certain aspects, provided herein are compositions comprising one or more PRAME immunogenic peptides described herein and an adjuvant.

### Table 1: PRAME Epitopes

TABLE 1A
PRAME epitopes presented by HLA serotype HLA-A*02
Peptide Epitopes

SLLQHLIGL

* Included in Table 1, such as Table 1A are peptide epitopes, as well as polypeptide molecules comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, 99.5%, or more identity across their full length with an amino acid sequence of any sequence listed in Table 1, such as Table 1A, or a portion thereof. Such polypeptides may have a function of the full-length peptide or polypeptide as described further herein.

In some embodiments, provided herein are PRAME polypeptides and/or nucleic acids encoding PRAME polypeptides. In some embodiments, PRAME polypeptides are polypeptides that include an amino acid sequence of sufficient length to elicit a PRAME-specific immune response. In certain embodiments, the PRAME polypeptide also includes amino acids that do not correspond to the amino acid sequence (e.g., a fusion protein comprising a PRAME amino acid sequence and an amino acid sequence corresponding to a non-PRAME protein or polypeptide). In some embodiments, the PRAME polypeptide only includes amino acid sequence corresponding to a PRAME protein or fragment thereof.

In some embodiments, the PRAME polypeptide has an amino acid sequence that comprises, consists essentially of, or consists of at least 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 373, or more, or any range in between inclusive (e.g., 7-25, 8-22, 9-22, etc.) consecutive amino acids of a PRAME protein amino acid sequence, such as those set forth in Table 3. In some embodiments, the consecutive amino acids are identical to an amino acid sequence of PRAME set forth in Table 3. In some embodiments, PRAME polypeptides comprise, consist essentially of, or consist of one or more peptide epitopes selected from the group consisting of PRAME peptide epitopes listed in Table 1, such as Table 1A.

As is well-known to those skilled in the art, polypeptides having substantial sequence similarities can cause identical or very similar immune reaction in a host animal. Accordingly, in some embodiments, a derivative, equivalent, variant, fragment, or mutant of a PRAME immunogenic peptide described herein or fragment thereof may also suitable for the methods and compositions provided herein.

In some embodiments, variations or derivatives of the PRAME immunogenic polypeptides are provided herein. The altered polypeptide may have an altered amino acid sequence, for example by conservative substitution, yet still elicits immune responses which react with the unaltered protein antigen, and are considered functional equivalents. As used herein, the term “conservative substitution” denotes the replacement of an amino acid residue by another, biologically similar residue. It is well-known in the art that the amino acids within the same conservative group may typically substitute for one another without substantially affecting the function of a protein. According to certain embodiments, the derivative, equivalents, variants, or mutants of the ligand-binding domain of a PRAME immunogenic peptide are polypeptides that are at least 85% homologous to the sequence of a PRAME immunogenic peptide described herein or fragment thereof. In some embodiments, the homology is at least 90%, at least 95%, at least 98%, or more.

Immunogenic peptides encompassed by the present invention may comprise a peptide epitope derived from a PRAME protein, such as those listed in Table 1, such as Table 1A. In some embodiments, the immunogenic peptide is 8, 9, 10, 1, 12, 13, 14, or 15 amino acids in length. In some embodiments, the peptide amino acid sequences is modified, which may include conservative or non-conservative mutations. A peptide may comprise at most 1, 2, 3, 4, or more mutations. In some embodiments, a peptide may comprise at least 1, 2, 3, 4, or more mutations.

In some embodiments, a peptide may be chemically modified. For example, a peptide can be mutated to modify peptide properties such as detectability, stability, biodistribution, pharmacokinetics, half-life, surface charge, hydrophobicity, conjugation sites, pH, function, and the like. N-methylation is one example of methylation that can occur in a peptide of the disclosure. In some embodiments, a peptide may be modified by methylation on free amines such as by reductive methylation with formaldehyde and sodium cyanoborohydride.

A chemical modification may comprise a polymer, a polyether, polyethylene glycol, a biopolymer, a zwitterionic polymer, a polyamino acid, a fatty acid, a dendrimer, an Fc region, a simple saturated carbon chain such as palmitate or myristoleate, or albumin. The chemical modification of a peptide with an Fc region may be a fusion Fc-peptide. A polyamino acid may include, for example, a poly amino acid sequence with repeated single amino acids (e.g., poly glycine), and a poly amino acid sequence with mixed poly amino acid sequences that may or may not follow a pattern, or any combination of the foregoing. In some embodiments, the peptides encompassed by the present disclosure may be modified such that the modification increases the stability and/or the half-life of the peptides. In some embodiments, the attachment of a hydrophobic moiety, such as to the N-terminus, the C-terminus, or an internal amino acid, can be used to extend half-life of a peptide encompassed by the present disclosure. In other embodiments, a peptide may include post-translational modifications (e.g., methylation and/or amidation), which can affect, for example, serum half-life. In some embodiments, simple carbon chains (e.g., by myristoylation and/or palmitoylation) can be conjugated to the fusion proteins or peptides. In some embodiments, the simple carbon chains may render the fusion proteins or peptides easily separable from the unconjugated material. For example, methods that may be used to separate the fusion proteins or peptides from the unconjugated material include, but are not limited to, solvent extraction and reverse phase chromatography. The lipophilic moieties can extend half-life through reversible binding to serum albumin. The conjugated moieties may be lipophilic moieties that extend half-life of the peptides through reversible binding to serum albumin. In some embodiments, the lipophilic moiety may be cholesterol or a cholesterol derivative, including cholestenes, cholestanes, cholestadienes and oxysterols. In some embodiments, the peptides may be conjugated to myristic acid (tetradecanoic acid) or a derivative thereof. In other embodiments, a peptide may be coupled (e.g., conjugated) to a half-life modifying agent. Examples of half-life modifying agents include but are not limited to: a polymer, a polyethylene glycol (PEG), a hydroxyethyl starch, polyvinyl alcohol, a water soluble polymer, a zwitterionic water soluble polymer, a water soluble poly(amino acid), a water soluble polymer of proline, alanine and serine, a water soluble polymer containing glycine, glutamic acid, and serine, an Fc region, a fatty acid, palmitic acid, or a molecule that binds to albumin. In some embodiments, a spacer or linker may be coupled to a peptide, such as 1, 2, 3, 4, or more amino acid residues that serve as a spacer or linker in order to facilitate conjugation or fusion to another molecule, as well as to facilitate cleavage of the peptide from such conjugated or fused molecules. In some embodiments, fusion proteins or peptides may be conjugated to other moieties that, for example, can modify or effect changes to the properties of the peptides.

A peptide may, in some embodiments, be covalently linked to a moiety. In some embodiments, the covalently linked moiety comprises an affinity tag or a label. The affinity tag may be selected from the group consisting of glutathione-S-transferase (GST), calmodulin binding protein (CBP), protein C tag, Myc tag, HaloTag, HA tag, Flag® tag, His tag, biotin tag, and V5 tag. The label may be a fluorescent protein. In some embodiments, the covalently linked moiety is selected from the group consisting of an inflammatory agent, an anti-inflammatory agent, a cytokine, a toxin, a cytotoxic molecule, a radioactive isotope, or an antibody such as a single-chain Fv.

A peptide may be conjugated to an agent used in imaging, research, therapeutics, theranostics, pharmaceuticals, chemotherapy, chelation therapy, targeted drug delivery, and radiotherapy. In some embodiments, a peptide may be conjugated to or fused with detectable agents, such as a fluorophore, a near-infrared dye, a contrast agent, a nanoparticle, a metal-containing nanoparticle, a metal chelate, an X-ray contrast agent, a PET agent, a metal, a radioisotope, a dye, radionuclide chelator, or another suitable material that can be used in imaging. In some embodiments, 1, 2, 3, 4, 5, 6, 7, 8, 9, 10, or more detectable moieties may be linked to a peptide. Non-limiting examples of radioisotopes include alpha emitters, beta emitters, positron emitters, and gamma emitters. In some embodiments, the metal or radioisotope is selected from the group consisting of actinium, americium, bismuth, cadmium, cesium, cobalt, europium, gadolinium, iridium, lead, lutetium, manganese, palladium, polonium, radium, ruthenium, samarium, strontium, technetium, thallium, and yttrium. In some embodiments, the metal is actinium, bismuth, lead, radium, strontium, samarium, or yttrium. In some embodiments, the radioisotope is actinium-225 or lead-212. In some embodiments, the near-infrared dyes are not easily quenched by biological tissues and fluids. In some embodiments, the fluorophore is a fluorescent agent emitting electromagnetic radiation at a wavelength between 650 nm and 4000 nm, such emissions being used to detect such agent. Non-limiting examples of fluorescent dyes that may be used as a conjugating molecule include DyLight®-680, DyLight®-750, VivoTag®-750, DyLight®-800, IRDye®-800, VivoTag®-680, Cy5.5, ZQ800, or indocyanine green (ICG). In some embodiments, near infrared dyes often include cyanine dyes (e.g., Cy7, Cy5.5, and Cy5). Additional non-limiting examples of fluorescent dyes for use as a conjugating molecule in the present disclosure include acridine orange or yellow, Alexa Fluors® (e.g., Alexa Fluor®790, 750, 700, 680, 660, and 647) and any derivative thereof, 7-actinomycin D, 8-anilinonaphthalene-1-sulfonic acid, ATTO dye and any derivative thereof, auramine-rhodamine stain and any derivative thereof, benzanthrone, bimane, 9-10-bis(phenylethynyl)anthracene, 5,12-bis(phenylethynyl)naphthacene, bisbenzimide, brainbow, calcein, carboxyfluorescein and any derivative thereof, 1-chloro-9,10-bis(phenylethynyl)anthracene and any derivative thereof, DAPI, DiOC6, DyLight Fluors and any derivative thereof, epicocconone, ethidium bromide. FlAsH-EDT2. Fluo dye and any derivative thereof, FluoProbe and any derivative thereof, Fluorescein and any derivative thereof. Fura and any derivative thereof, GelGreen and any derivative thereof, GelRed and any derivative thereof, fluorescent proteins and any derivative thereof, m isoform proteins and any derivative thereof such as for example mCherry, heptamethine dye and any derivative thereof, hoeschst stain, iminocoumarin, indian yellow, indo-1 and any derivative thereof, laurdan, lucifer yellow and any derivative thereof, luciferin and any derivative thereof, luciferase and any derivative thereof, merocyanine and any derivative thereof, nile dyes and any derivative thereof, perylene, phloxine, phyco dye and any derivative thereof, propium iodide, pyranine, rhodamine and any derivative thereof, ribogreen, RoGFP, rubrene, stilbene and any derivative thereof, sulforhodamine and any derivative thereof, SYBRT™ and any derivative thereof, synapto-pHluorin, tetraphenyl butadiene, tetrasodium tris, Texas Red, Titan Yellow, TSQ, umbelliferone, violanthrone, yellow fluorescent protein and YOYO-1. Other Suitable fluorescent dyes include, but are not limited to, fluorescein and fluorescein dyes (e.g., fluorescein isothiocyanine or FITC, naphthofluorescein, 4′,5′-dichloro-2′,7′-dimethoxyfluorescein, 6-carboxyfluorescein or FAM, etc.), carbocyanine, merocyanine, styryl dyes, oxonol dyes, phycoerythrin, erythrosin, eosin, rhodamine dyes (e.g., carboxytetramethyl-rhodamine or TAMRA, carboxyrhodamine 6G, carboxy-X-rhodamine (ROX), lissamine rhodamine B, rhodamine 6G, rhodamine Green, rhodamine Red, tetramethylrhodamine (TMR), etc.), coumarin and coumarin dyes (e.g., methoxycoumarin, dialkylaminocoumarin, hydroxycoumarin, aminomethylcoumarin (AMCA), etc.), Oregon Green® Dyes (e.g., Oregon Green® 488, Oregon Green® 500, Oregon Green® 514., etc.), Texas Red, Texas Red-X, SPECTRUM RED, SPECTRUM GREEN, cyanine dyes (e.g., CY-3, Cy-5, CY-3.5, CY-5.5, etc.), ALEXA FLUOR® dyes (e.g., ALEXA FLUOR®350, ALEXA FLUOR® 488, ALEXA FLUOR®532, ALEXA FLUOR®546, ALEXA FLUOR® 568. ALEXA FLUOR®594, ALEXA FLUOR® 633, ALEXA FLUOR®660, ALEXA FLUOR® 680, etc.), BODIPY® dyes (e.g., BODIPY® FL, BODIPY® R6G, BODIPY® TMR, BODIPY® TR, BODIPY® 530/550, BODIPY® 558/568, BODIPY® 564/570, BODIPY® 576/589, BODIPY® 581/591, BODIPY® 630/650, BODIPY® 650/665, etc.), IRDyes (e.g., IRD40, IRD 700, IRD 800, etc.), and the like. Additional suitable detectable agents are described in PCT/US14/56177. Non-limiting examples of radioisotopes include alpha emitters, beta emitters, positron emitters, and gamma emitters. In some embodiments, the metal or radioisotope is selected from the group consisting of actinium, americium, bismuth, cadmium, cesium, cobalt, europium, gadolinium, iridium, lead, lutetium, manganese, palladium, polonium, radium, ruthenium, samarium, strontium, technetium, thallium, and yttrium. In some embodiments, the metal is actinium, bismuth, lead, radium, strontium, samarium, or yttrium. In some embodiments, the radioisotope is actinium-225 or lead-212.

A peptide may be conjugated to a radiosensitizer or photosensitizer. Examples of radiosensitizers include but are not limited to: ABT-263, ABT-199, WEHI-539, paclitaxel, carboplatin, cisplatin, oxaliplatin, gemcitabine, etanidazole, misonidazole, tirapazamine, and nucleic acid base derivatives (e.g., halogenated purines or pyrimidines, such as 5-fluorodeoxyuridine). Examples of photosensitizers include but are not limited to: fluorescent molecules or beads that generate heat when illuminated, nanoparticles, porphyrins and porphyrin derivatives (e.g., chlorins, bacteriochlorins, isobacteriochlorins, phthalocyanines, and naphthalocyanines), metalloporphyrins, metallophthalocyanines, angelicins, chalcogenapyrylium dyes, chlorophylls, coumarins, flavins and related compounds such as alloxazine and riboflavin, fullerenes, pheophorbides, pyropheophorbides, cyanines (e.g., merocyanine 540), pheophytins, sapphyrins, texaphyrins, purpurins, porphycenes, phenothiaziniums, methylene blue derivatives, naphthalimides, nile blue derivatives, quinones, perylenequinones (e.g., hypericins, hypocrellins, and cercosporins), psoralens, quinones, retinoids, rhodamines, thiophenes, verdins, xanthene dyes (e.g., eosins, erythrosins, rose bengals), dimeric and oligomeric forms of porphyrins, and prodrugs such as 5-aminolevulinic acid. Advantageously, this approach allows for highly specific targeting of cells of interest (e.g., immune cells) using both a therapeutic agent (e.g., drug) and electromagnetic energy (e.g., radiation or light) concurrently. In some embodiments, the peptide is fused with, or covalently or non-covalently linked to the agent, for example, directly or via a linker.

In some embodiments, the binding protein may be chemically modified. For example, a binding protein may be mutated to modify peptide properties such as detectability, stability, biodistribution, pharmacokinetics, half-life, surface charge, hydrophobicity, conjugation sites, pH, function, and the like. N-methylation is one example of methylation that can occur in a binding protein encompassed by the present invention. In some embodiments, a binding protein may be modified by methylation on free amines such as by reductive methylation with formaldehyde and sodium cyanoborohydride.

A chemical modification may comprise a polymer, a polyether, polyethylene glycol, a biopolymer, a zwitterionic polymer, a polyamino acid, a fatty acid, a dendrimer, an Fc region, a simple saturated carbon chain such as palmitate or myristoleate, or albumin. The chemical modification of a binding protein with an Fc region may be a fusion Fc-protein. A polyamino acid may include, for example, a poly amino acid sequence with repeated single amino acids (e.g., poly glycine), and a poly amino acid sequence with mixed poly amino acid sequences that may or may not follow a pattern, or any combination of the foregoing.

In some embodiments, the binding proteins encompassed by the present invention may be modified. In some embodiments, the modifications having substantial or significant sequence identity to a parent binding protein to generate a functional variant that maintains one or more biophysical and/or biological activities of the parent binding protein (e.g., maintain pMHC binding specificity). In some embodiments, the mutation is a conservative amino acid substitution.

In some embodiments, binding proteins encompassed by the present invention may comprise synthetic amino acids in place of one or more naturally-occurring amino acids. Such synthetic amino acids are well-known in the art, and include, for example, aminocyclohexane carboxylic acid, norleucine, α-amino n-decanoic acid, homoserine, S-acetylaminomethyl-cysteine, trans-3- and trans-4-hydroxyproline, 4-aminophenylalanine, 4-nitrophenylalanine, 4-chlorophenylalanine, 4-carboxyphenylalanine. β-phenylserine β-hydroxyphenylalanine, phenylglycine, α-naphthylalanine, cyclohexylalanine, cyclohexylglycine, indoline-2-carboxylic acid, 1,2,3,4-tetrahydroisoquinoline-3-carboxylic acid, aminomalonic acid, aminomalonic acid monoamide, N′-benzyl-N′-methyl-lysine, N′,N′-dibenzyl-lysine, 6-hydroxylysine, ornithine, a-aminocyclopentane carboxylic acid, oc-aminocyclohexane carboxylic acid, a-aminocycloheptane carboxylic acid, a-(2-amino-2-norbornane)-carboxylic acid, α,γ-diaminobutyric acid, β-diaminopropionic acid, homophenylalanine, and oc-tert-butylglycine.

Binding proteins encompassed by the present invention may be glycosylated, amidated, carboxylated, phosphorylated, esterified. N-acylated, cyclized (e.g., via a disulfide bridge), or converted into an acid addition salt and/or optionally dimerized or polymerized, or conjugated.

In some embodiments, the attachment of a hydrophobic moiety, such as to the N-terminus, the C-terminus, or an internal amino acid, may be used to extend half-life of a peptide encompassed by the present invention. In other embodiments, a binding protein may include post-translational modifications (e.g., methylation and/or amidation), which can affect, for example, serum half-life. In some embodiments, simple carbon chains (e.g., by myristoylation and/or palmitoylation) may be conjugated to the binding proteins. In some embodiments, the simple carbon chains may render the binding proteins easily separable from the unconjugated material. For example, methods that may be used to separate the binding proteins from the unconjugated material include, but are not limited to, solvent extraction and reverse phase chromatography. The lipophilic moieties can extend half-life through reversible binding to serum albumin. The conjugated moieties may be lipophilic moieties that extend half-life of the peptides through reversible binding to serum albumin. In some embodiments, the lipophilic moiety may be cholesterol or a cholesterol derivative, including cholestenes, cholestanes, cholestadienes and oxysterols. In some embodiments, the binding proteins may be conjugated to myristic acid (tetradecanoic acid) or a derivative thereof. In other embodiments, a binding protein may be coupled (e.g., conjugated) to a half-life modifying agent. Examples of half-life modifying agents include but are not limited to: a polymer, a polyethylene glycol (PEG), a hydroxyethyl starch, polyvinyl alcohol, a water soluble polymer, a zwitterionic water soluble polymer, a water soluble poly(amino acid), a water soluble polymer of proline, alanine and serine, a water soluble polymer containing glycine, glutamic acid, and serine, an Fc region, a fatty acid, palmitic acid, or a molecule that binds to albumin. In some embodiments, a spacer or linker may be coupled to a binding protein, such as 1, 2, 3, 4, or more amino acid residues that serve as a spacer or linker in order to facilitate conjugation or fusion to another molecule, as well as to facilitate cleavage of the peptide from such conjugated or fused molecules. In some embodiments, binding proteins may be conjugated to other moieties that, for example, can modify or effect changes to the properties of the binding proteins.

A protein such as a peptide may be produced recombinantly or synthetically, such as by solid-phase peptide synthesis or solution-phase peptide synthesis. Protein synthesis may be performed by known synthetic methods, such as using fluorenylmethyloxycarbonyl (Fmoc) chemistry or by butyloxycarbonyl (Boc) chemistry. Protein fragments may be joined together enzymatically or synthetically.

In an aspect encompassed by the present invention, provided herein are methods of producing a protein described herein, comprising the steps of: (i) culturing a transformed host cell which has been transformed by a nucleic acid comprising a sequence encoding a binding protein described herein under conditions suitable to allow expression of said binding protein; and (ii) recovering the expressed binding protein.

Methods useful for isolating and purifying recombinantly produced binding protein, by way of example, may include obtaining supernatants from suitable host cell/vector systems that secrete the binding protein into culture media and then concentrating the media using a commercially available filter. Following concentration, the concentrate may be applied to a single suitable purification matrix or to a series of suitable matrices, such as an affinity matrix or an ion exchange resin. One or more reverse phase HPLC steps may be employed to further purify a recombinant polypeptide. These purification methods may also be employed when isolating an immunogen from its natural environment. Methods for large scale production of one or more of binding proteins described herein include batch cell culture, which is monitored and controlled to maintain appropriate culture conditions. Purification of the binding protein may be performed according to methods described herein and known in the art.

In some embodiments, provided herein is a nucleic acid encoding a PRAME immunogenic peptide described herein or fragment thereof, such as a DNA molecule encoding a PRAME immunogenic peptide. In some embodiments, the composition comprises an expression vector comprising an open reading frame encoding a PRAME immunogenic peptide described herein or fragment thereof. In some embodiments, the nucleic acid includes regulatory elements necessary for expression of the open reading frame. Such elements may include, for example, a promoter, an initiation codon, a stop codon, and a polyadenylation signal. In addition, enhancers may be included. These elements may be operably linked to a sequence that encodes the PRAME immunogenic polypeptide or fragment thereof. Representative vectors, promoters, regulatory elements, and the like useful for expressing proteins such as peptide are described further below.

### III. MHC-Peptide Complexes

In certain aspects, provided herein are compositions comprising a PRAME immunogenic peptide described herein and a MHC molecule. In some embodiments, the PRAME immunogenic peptide forms a stable complex with the MHC molecule.

MHC proteins may be conjugated to an agent, such as a detection moiety, radiosensitizer, photosensitizer, and the like, and/or may be chemically modified as described above regarding peptides.

The MHC proteins provided and used in the compositions and methods encompassed by the present disclosure may be any suitable MHC molecules known in the art. Generally, they have the formula (α-β-P)ₙ, where n is at least 2, for example between 2-10, e.g., 4. α is an α chain of a class I or class II MHC protein. β is a β chain, herein defined as the β chain of a class II MHC protein or β₂ microglobulin for a MHC class I protein. P is a peptide antigen.

In some embodiments, the MHC proteins are MHC class I complexes, such as HLA I complexes.

The MHC proteins may be from any mammalian or avian species. e.g., primate sp., particularly humans; rodents, including mice, rats and hamsters; rabbits; equines, bovines, canines, felines; etc. For instance, the MHC protein may be derived the human HLA proteins or the murine H-2 proteins. HLA proteins include the class II subunits HLA-DPα, HLA-DPβ, HLA-DQα, HLA-DQβ, HLA-DRα and HLA-DRβ, and the class I proteins HLA-A, HLA-B, HLA-C, and β2-microglobulin. H-2 proteins include the class I subunits H-2K, H-2D, H-2L, and the class II subunits I-Aα, I-Aβ, I-Eα and I-Eβ, and β2-microglobulin.

Sequences of some representative MHC proteins may be found in Kabat et al. Sequences of Proteins of Immunological Interest, NIH Publication No. 91-3242, pp 724-815. MHC protein subunits suitable for use in the present invention are a soluble form of the normally membrane-bound protein, which is prepared as known in the art, for instance by deletion of the transmembrane domain and the cytoplasmic domain.

For class I proteins, the soluble form may include the α1, α2 and α3 domain. Soluble class II subunits may include the α1 and α2 domains for the a subunit, and the β1 and β2 domains for the β subunit.

The α and β subunits may be separately produced and allowed to associate in vitro to form a stable heteroduplex complex, or both of the subunits may be expressed in a single cell. Methods for producing MHC subunits are known in the art.

In certain embodiments, the MHC-peptide complex comprises a peptide epitope selected from Table 1 and an MHC. In some embodiments, the MHC molecule comprises an MHC alpha chain that is an HLA serotype selected from the group consisting of HLA-A*02, HLA-A*03, HLA-A*01, HLA-A*11, HLA-A*24, HLA-B*07, HLA-C*07, HLA-C*01, HLA-C*02, HLA-C*03, HLA-C*04, HLA-C*05, HLA-C*06, HLA-C*08, HLA-C*12, HLA-C*14, HLA-C*15, HLA-C*16, HLA-C*17, and HLA-C*18, optionally wherein the HLA allele is selected from the group consisting of HLA-A*02:01, HLA-A*02:02, HLA-A*02:03, HLA-A*02:04, HLA-A*02:05, HLA-A*02:06, HLA-A*02:07, HLA-A*02:10, HLA-A*02:11, HLA-A*02:12, HLA-A*02:13, HLA-A*02:14, HLA-A*02:16, HLA-A*02:17, HLA-A*02:19, HLA-A*02:20, HLA-A*02:22, HLA-A*02:24, HLA-A*02:30, HLA-A*02:42, HLA-A*02:53, HLA-A*02:60, HLA-A*02:74 allele, HLA-A*03:01, HLA-A*03:02, HLA-A*03:05, HLA-A*03:07, HLA-A*01:01, HLA-A*01:02, HLA-A*01:03. HLA-A*01:16 allele, HLA-A*l 1:01, HLA-A*11:02, HLA-A*l 1:03, HLA-A*11:04, HLA-A*11:05, HLA-A*11:19 allele, HLA-A*24:02, HLA-A*24:03, HLA-A*24:05, HLA-A*24:07, HLA-A*24:08, HLA-A*24:10, HLA-A*24:14, HLA-A*24:17, HLA-A*24:20, HLA-A*24:22, HLA-A*24:25, HLA-A*24:26, HLA-A*24:58 allele, HLA-B*07:02, HLA-B*07:04, HLA-B*07:05, HLA-B*07:09, HLA-B*07:10, HLA-B*07:15, HLA-B*07:21, HLA-C*07:02, HLA-C*07:01, HLA-C*04:01, HLA-C*06:02, HLA-C*03:04, HLA-C*05:01, HLA-C*16:01, HLA-C*02:02, HLA-C*03:03, HLA-C*12:03, HLA-C*08:02, HLA-C*01:02, HLA-C*17:01, HLA-C*15:02, HLA-C*14:02, HLA-C*12:02, HLA-C*07:04, HLA-C*08:01, HLA-C*03:02, HLA-C*18:01, HLA-C*15:05, HLA-C*16:02, HLA-C*08:04, HLA-C*03:05, and HLA-C*14:03 allele. In some embodiments, the MHC-peptide complex comprises a peptide epitope selected from Table 1A and an MHC whose alpha chain has an HLA-A*02 serotype, such as that encoded by an HLA-A*02:01 allele.

To prepare the MHC-peptide complex, the subunits may be combined with an antigenic peptide and allowed to fold in vitro to form a stable heterodimer complex with intrachain disulfide bonded domains. The peptide may be included in the initial folding reaction, or may be added to the empty heterodimer in a later step. In the compositions and methods encompassed by the present invention, this is a PRAME immunogenic peptide or fragment thereof. Conditions that permit folding and association of the subunits and peptide are known in the art. As one example, roughly equimolar amounts of solubilized α and β subunits may be mixed in a solution of urea. Refolding is initiated by dilution or dialysis into a buffered solution without urea. Peptides may be loaded into empty class II heterodimers at about pH 5 to 5.5 for about 1 to 3 days, followed by neutralization, concentration and buffer exchange. However, the specific folding conditions are not critical for the practice of the invention.

The monomeric complex (α-β-P) (herein monomer) may be multimerized, for example, for a MHC tetramer. The resulting multimer is stable over long periods of time. Preferably, the multimer may be formed by binding the monomers to a multivalent entity through specific attachment sites on the α or β subunit, as known in the art (e.g., as described in U.S. Pat. No. 5,635,363). The MHC proteins, in either their monomeric or multimeric forms, may also be conjugated to beads or any other support.

The multimeric complex may be labeled, so as to be directly detectable when used in immunostaining or other methods known in the art, or may be used in conjunction with secondary labeled immunoreagents which specifically and/or selectively bind the complex (e.g., bind to a MHC protein subunit) as known in the art. For example, the detectable label may be a fluorophore, such as fluorescein isothiocyanate (FITC), rhodamine, Texas Red, phycoerythrin (PE), allophycocyanin (APC), Brilliant Violet™ 421, Brilliant UV™ 395, Brilliant Violet™ 480, Brilliant Violet™ 421 (BV421), Brilliant Blue™ 515, APC-R700, or APC-Fire750. In some embodiments, the multimeric complex is labeled by a moiety that is capable of specifically and/or selectively binding another moiety. For instance, the label may be biotin, streptavidin, an oligonucleotide, or a ligand. Other labels of interest may include fluorochromes, dyes, enzymes, chemiluminescers, particles, radioisotopes, or other directly or indirectly detectable agent.

In some embodiments, a cell presenting an immunogenic peptides in context of an MHC molecule on the cell surface is generated by transfecting or transducing the cell with a vector (e.g., a viral vector) that comprising nucleic acid that encodes a recombinant or heterologous antigen into a cell. In some embodiments, the vector is introduced into the cell under conditions in which one or more peptide antigens, including, in some cases, one or more peptide antigens of the expressed heterologous protein, are expressed by the cell, processed and presented on the surface of the cell in the context of a major histocompatibility complex (MHC) molecule.

Generally, the cell to which the vector is contacted is a cell that expresses MHC, i.e., MHC-expressing cells. The cell may be one that normally expresses an MHC on the cell surface, that is induced to express and/or upregulate expression of MHC on the cell surface or that is engineered to express an MHC molecule on the cell surface. In some embodiments, the MHC contains a polymorphic peptide binding site or binding groove that can, in some cases, complex with peptide antigens of polypeptides, including peptide antigens processed by the cell machinery. In some cases, MHC molecules may be displayed or expressed on the cell surface, including as a complex with peptide, i.e., MHC-peptide complex, for presentation of an antigen in a conformation recognizable by TCRs on T cells, or other peptide binding molecules.

In some embodiments, the cell is a nucleated cell. In some embodiments, the cell is an antigen-presenting cell. In some embodiments, the cell is a macrophage, dendritic cell. B cell, endothelial cell or fibroblast. In some embodiments, the cell is an endothelial cell, such as an endothelial cell line or primary endothelial cell. In some embodiments, the cell is a fibroblast, such as a fibroblast cell line or a primary fibroblast cell.

In some embodiments, the cell is an artificial antigen presenting cell (aAPC). Typically, aAPCs include features of natural APCs, including expression of an MHC molecule, stimulatory and costimulatory molecule(s). Fc receptor, adhesion molecule(s) and/or the ability to produce or secrete cytokines (e.g., IL-2). Normally, an aAPC is a cell line that lacks expression of one or more of the above, and is generated by introduction (e.g., by transfection or transduction) of one or more of the missing elements from among an MHC molecule, a low affinity Fc receptor (CD32), a high affinity Fc receptor (CD64), one or more of a co-stimulatory signal (e.g., CD7, B7-1 (CD80), B7-2 (CD86), PD-L1, PD-L2, 4-1BBL, OX40L, ICOS-L, ICAM, CD30L, CD40, CD70, CD83, HLA-G, MICA, MICB, HVEM, lymphotoxin beta receptor, ILT3, ILT4, 3/TR6 or a ligand of B7-H3; or an antibody that specifically binds to CD27, CD28, 4-1BB, OX40, CD30, CD40, PD-1, ICOS, LFA-1, CD2, CD7, LIGHT, NKG2C, B7-H3, Toll ligand receptor or a ligand of CD83), a cell adhesion molecule (e.g., ICAM-1 or LFA-3) and/or a cytokine (e.g., IL-2, IL-4, IL-6, IL-7, IL-10, IL-12, IL-15. IL-21, interferon-alpha (IFN.alpha.), interferon-beta (IFN.beta.), interferon-gamma (IFN.gamma.), tumor necrosis factor-alpha (TNF.alpha.), tumor necrosis factor-beta (TNF.beta.), granulocyte macrophage colony stimulating factor (GM-CSF), and granulocyte colony stimulating factor (GCSF)). In some cases, an aAPC does not normally express an MHC molecule, but may be engineered to express an MHC molecule or, in some cases, is or may be induced to express an MHC molecule, such as by stimulation with cytokines. In some cases, aAPCs also may be loaded with a stimulatory ligand, which may include, for example, an anti-CD3 antibody, an anti-CD28 antibody or an anti-CD2 antibody. An exemplary cell line that may be used as a backbone for generating an aAPC is a K562 cell line or a fibroblast cell line. Various aAPCs are known in the art, see e.g., U.S. Pat. No. 8,722,400, published application No. US2014/0212446; Butler and Hirano (2014) Immunol Rev, 257:10. 1111/imr.12129; Suhoshki et al. (2007) Mol. Ther, 15:981-988).

It is well within the level of a skilled artisan to determine or identify the particular MHC or allele expressed by a cell. In some embodiments, prior to contacting cells with a vector, expression of a particular MHC molecule may be assessed or confirmed, such as by using an antibody specific for the particular MHC molecule. Antibodies to MHC molecules are known in the art, such as any described below.

In some embodiments, the cells may be chosen to express an MHC allele of a desired MHC restriction. In some embodiments, the MHC typing of cells, such as cell lines, are well-known in the art. In some embodiments, the MHC typing of cells, such as primary cells obtained from a subject, may be determined using procedures well-known in the art, such as by performing tissue typing using molecular haplotype assays (BioTest ABC SSPtray, BioTest Diagnostics Corp., Denville, N.J.; SeCore Kits, Life Technologies. Grand Island, N.Y.). In some cases, it is well within the level of a skilled artisan to perform standard typing of cells to determine the HLA genotype, such as by using sequence-based typing (SBT) (Adams et al. (2004) J. Transl. Med., 2:30; Smith (2012) Methods Mol Biol., 882:67-86). In some cases, the HLA typing of cells, such as fibroblast cells, are known. For example, the human fetal lung fibroblast cell line MRC-5 is HLA-A*02:01, A29, B13. B44 Cw7 (C*0702); the human foreskin fibroblast cell line Hs68 is HLA-A1, A29, B8, B44, Cw7, Cw16; and the WI-38 cell line is A*68:01, B*08:01. (Solache et al. (1999) J Immunol, 163:5512-5518; Ameres et al. (2013) PloS Pathog, 9:e1003383). The human transfectant fibroblast cell line M1DR1/Ii/DM express HLA-DR and HLA-DM (Karakikes et al. (2012) FASEB J., 26:4886-96).

In some embodiments, the cells to which the vector is contacted or introduced are cells that are engineered or transfected to express an MHC molecule. In some embodiments, cell lines may be prepared by genetically modifying a parental cells line. In some embodiments, the cells are normally deficient in the particular MHC molecule and are engineered to express such particular MHC molecule. In some embodiments, the cells are genetically engineered using recombinant DNA techniques.

In some embodiments, the stable MHC-peptide complexes described herein are used to detect T cells that bind a stable MHC-peptide complex. In some embodiments, the stable MHC-peptide complexes described herein are used to monitor T cell response in a subject, for example, by detecting the amount and/or percentage of T cells (e.g., CD8+ T cells) that specifically and/or selectively bind to the MHC-peptide complexes that are fluorescently labeled. Methods of generating, labeling, and using MHC-peptide complexes (e.g., MHC-peptide tetramers) for detecting MHC-peptide complex-specific T cells are well-known in the art. Additional description can be found in, for example, U.S. Pat. Nos. 7,776,562; 8,268,964; and U.S. Pat. Publ, 2019/0085048.

### IV. Immunogenic Compositions

In some aspects, provided herein are pharmaceutical compositions (e.g., a vaccine composition) comprising a PRAME immunogenic peptide and/or a nucleic acid encoding a PRAME immunogenic peptide and an adjuvant. In some aspects, provided herein are pharmaceutical compositions (e.g., a vaccine composition) comprising a stable MHC-peptide complex comprising a PRAME immunogenic peptide in the context of a MHC molecule and an adjuvant. In some embodiments, the composition includes a combination of multiple (e.g., two or more) PRAME immunogenic peptides or nucleic acids and an adjuvant. In some embodiments, the composition includes a combination of multiple (e.g., two or more) stable MHC-peptide complexes comprising a PRAME immunogenic peptide in the context of a MHC molecule and an adjuvant. In some embodiments, the compositions described above further comprises a pharmaceutically acceptable carrier.

The pharmaceutical compositions disclosed herein may be specially formulated for administration in solid or liquid form, including those adapted for the following: (1) oral administration, for example, drenches (aqueous or non-aqueous solutions or suspensions), tablets, e.g., those targeted for buccal, sublingual, and systemic absorption, boluses, powders, granules, pastes for application to the tongue; or (2) parenteral administration, for example, by subcutaneous, intramuscular, intravenous or epidural injection as, for example, a sterile solution or suspension, or sustained-release formulation.

Methods of preparing these formulations or compositions include the step of bringing into association a PRAME immunogenic peptide and/or nucleic acid described herein with the adjuvant, carrier and, optionally, one or more accessory ingredients. In general, the formulations are prepared by uniformly and intimately bringing into association an agent described herein with liquid carriers, or finely divided solid carriers, or both, and then, if necessary, shaping the product.

Pharmaceutical compositions suitable for parenteral administration comprise PRAME immunogenic peptides and/or nucleic acids described herein in combination with a adjuvant, as well as one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain sugars, alcohols, antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity may be maintained, for example, by the use of coating materials, such as lecithin, by the maintenance of the required particle size in the case of dispersions, and by the use of surfactants.

Regardless of the route of administration selected, the agents provided herein, which may be used in a suitable hydrated form, and/or the pharmaceutical compositions disclosed herein, are formulated into pharmaceutically-acceptable dosage forms by conventional methods known to those of skill in the art.

In some embodiments, the pharmaceutical composition described, when administered to a subject, can elicit an immune response against a cell that is infected by PRAME. Such pharmaceutical compositions may be useful as vaccine compositions for prophylactic and/or therapeutic treatment of disorders characterized by PRAME expression.

In some embodiments, the pharmaceutical composition further comprises a physiologically acceptable adjuvant. In some embodiments, the adjuvant employed provides for increased immunogenicity of the pharmaceutical composition. Such a further immune response stimulating compound or adjuvant may be (i) admixed to the pharmaceutical composition according to the invention after reconstitution of the peptides and optional emulsification with an oil-based adjuvant as defined above, (ii) may be part of the reconstitution composition of the invention defined above, (iii) may be physically linked to the peptide(s) to be reconstituted or (iv) may be administered separately to the subject, mammal or human, to be treated. The adjuvant may be one that provides for slow release of antigen (e.g., the adjuvant may be a liposome), or it may be an adjuvant that is immunogenic in its own right thereby functioning synergistically with antigens (i.e., antigens present in the PRAME immunogenic peptide). For example, the adjuvant may be a known adjuvant or other substance that promotes antigen uptake, recruits immune system cells to the site of administration, or facilitates the immune activation of responding lymphoid cells. Adjuvants include, but are not limited to, immunomodulatory molecules (e.g., cytokines), oil and water emulsions, aluminum hydroxide, glucan, dextran sulfate, iron oxide, sodium alginate, Bacto-Adjuvant, synthetic polymers such as poly amino acids and co-polymers of amino acids, saponin, paraffin oil, and muramyl dipeptide. In some embodiments, the adjuvant is Adjuvant 65, α-GalCer, aluminum phosphate, aluminum hydroxide, calcium phosphate, β-Glucan Peptide, CpG DNA, GM-CSF, GPI-0100, IFA, IFN-γ, IL-17, lipid A, lipopolysaccharide, Lipovant, Montanide, N-acetyl-muramyl-L-alanyl-D-isoglutamine, Pam3CSK4, quil A, trehalose dimycolate or zymosan.

In some embodiments, the adjuvant is an immunomodulatory molecule. For example, the immunomodulatory molecule may be a recombinant protein cytokine, chemokine, or immunostimulatory agent or nucleic acid encoding cytokines, chemokines, or immunostimulatory agents designed to enhance the immunologic response.

Examples of immunomodulatory cytokines include interferons (e.g., IFNα, IFNβ and IFNγ), interleukins (e.g., IL-1, IL-2, IL-3, IL-4, IL-5, IL-6, IL-7, IL-8, IL-9, IL-10, IL-12. IL-17 and IL-20), tumor necrosis factors (e.g., TNFα and TNFβ), erythropoietin (EPO), FLT-3 ligand, gIp10, TCA-3, MCP-1, MIF, MIP-1.alpha., MIP-1β, Rantes, macrophage colony stimulating factor (M-CSF), granulocyte colony stimulating factor (G-CSF), and granulocyte-macrophage colony stimulating factor (GM-CSF), as well as functional fragments of any of the foregoing.

In some embodiments, an immunomodulatory chemokine that binds to a chemokine receptor, i.e., a CXC, CC, C, or CX3C chemokine receptor, also may be included in the compositions provided here. Examples of chemokines include, but are not limited to, Mip1α, Mip-1β, Mip-3α (Larc). Mip-3β, Rantes, Hcc-1, Mpif-1, Mpif-2, Mcp-1, Mcp-2, Mcp-3, Mcp-4, Mcp-5, Eotaxin, Tarc, Elc, 1309, IL-8, Gcp-2 Gro-α, Gro-β, Gro-γ, Nap-2, Ena-78, Gcp-2, Ip-10, Mig, I-Tac, Sdf-1, and Bca-1 (Blc), as well as functional fragments of any of the foregoing.

In some embodiments, the composition comprises a nucleic acid encoding an PRAME immunogenic polypeptide described herein, such as a DNA molecule encoding a PRAME immunogenic peptide. In some embodiments the composition comprises an expression vector comprising an open reading frame encoding a PRAME immunogenic peptide.

When taken up by a cell (e.g., host cell, an antigen-presenting cell (APC) such as a dendritic cell, macrophage, etc.), a DNA molecule may be present in the cell as an extrachromosomal molecule and/or may integrate into the chromosome. DNA may be introduced into cells in the form of a plasmid which may remain as separate genetic material. Alternatively, linear DNAs that may integrate into the chromosome may be introduced into the cell. Optionally, when introducing DNA into a cell, reagents which promote DNA integration into chromosomes may be added.

### V. Binding Proteins

In some aspects, a binding moiety that binds a peptide described herein and/or a stable MHC-peptide complex described herein, are provided. For example, binding proteins like T cell receptors (TCRs), antibodies, and the like that specifically and/or selectively bind to the peptide and/or the stable MHC-peptide complex, such as with a K_{d} less than or equal to about 10⁻⁴ M (e.g., about 10⁻⁴, 10⁻⁵, 10⁻⁶, 10⁻⁷, about 10⁻⁸, about 10⁻⁹ about 10¹⁰, about 10⁻¹¹, about 10⁻¹², about 10⁻¹³, about 10⁻¹⁴, etc.), are provided.

In an aspect encompassed by the present invention, provided herein are binding proteins that bind (e.g., specifically and/or selectively) to a peptide-MHC (pMHC) complex comprising a PRAME immunogenic peptide in the context of an MHC molecule (e.g., a MHC class I molecule). In some embodiments, the binding protein is capable of binding (e.g., specifically and/or selectively) to a PRAME peptide-MHC (pMHC) complex with a K_{d} less than or equal to about 5×10⁻⁴ M, less than or equal to about 1×10⁻⁴ M, less than or equal to about 5×10⁻⁵ M, less than or equal to about 1×10⁻⁵ M, less than or equal to about 5×10⁻⁶ M, less than or equal to about 1×10⁻⁶ M, less than or equal to about 5×10⁻⁷ M, less than or equal to about 1×10⁻⁷ M, less than or equal to about 5×10⁻⁸ M, less than or equal to about 1×10⁻⁸ M, less than or equal to about 5×10⁻⁹ M, less than or equal to about 1×10⁻⁹ M, less than or equal to about 5×10⁻¹⁰ M, less than or equal to about 1×10⁻¹⁰ M, less than or equal to about 5×10⁻¹¹ M, less than or equal to about 1×10⁻¹¹ M, less than or equal to about 5×10⁻¹² M, less than or equal to about 1×10⁻¹² M, or any range in between, inclusive, such as between about 1-50 micromolar, 1-100 micromolar, 0.1-500 micromolar, and the like. In some embodiments, the MHC molecule comprises an MHC alpha chain that is an HLA serotype selected from the group consisting of HLA-A*02, HLA-A*03, HLA-A*01, HLA-A*11, HLA-A*24, HLA-B*07, HLA-C*07, HLA-C*01, HLA-C*02, HLA-C*03, HLA-C*04, HLA-C*05, HLA-C*06, HLA-C*08, HLA-C*12, HLA-C*14, HLA-C*15, HLA-C*16, HLA-C*17, and HLA-C*18, optionally wherein the HLA allele is selected from the group consisting of HLA-A*02:01, HLA-A*02:02, HLA-A*02:03, HLA-A*02:04, HLA-A*02:05, HLA-A*02:06, HLA-A*02:07, HLA-A*02:10, HLA-A*02:11, HLA-A*02:12, HLA-A*02:13, HLA-A*02:14, HLA-A*02:16, HLA-A*02:17, HLA-A*02:19, HLA-A*02:20, HLA-A*02:22, HLA-A*02:24, HLA-A*02:30, HLA-A*02:42, HLA-A*02:53, HLA-A*02:60, HLA-A*02:74 allele, HLA-A*03:01, HLA-A*03:02, HLA-A*03:05, HLA-A*03:07, HLA-A*01:01, HLA-A*01:02, HLA-A*01:03, HLA-A*01:16 allele, HLA-A*11:01, HLA-A*11:02, HLA-A*l 1:03, HLA-A*11:04, HLA-A*l 1:05, HLA-A*11:19 allele, HLA-A*24:02, HLA-A*24:03, HLA-A*24:05, HLA-A*24:07, HLA-A*24:08, HLA-A*24:10, HLA-A*24:14, HLA-A*24:17, HLA-A*24:20, HLA-A*24:22, HLA-A*24:25, HLA-A*24:26, HLA-A*24:58 allele, HLA-B*07:02, HLA-B*07:04, HLA-B*07:05, HLA-B*07:09, HLA-B*07:10, HLA-B*07:15, HLA-B*07:21, HLA-C*07:02, HLA-C*07:01, HLA-C*04:01, HLA-C*06:02, HLA-C*03:04, HLA-C*05:01, HLA-C*16:01, HLA-C*02:02, HLA-C*03:03, HLA-C*12:03, HLA-C*08:02, HLA-C*01:02, HLA-C*17:01, HLA-C*15:02, HLA-C*14:02, HLA-C*12:02, HLA-C*07:04, HLA-C*08:01, HLA-C*03:02, HLA-C*18:01, HLA-C*15:05, HLA-C*16:02, HLA-C*08:04, HLA-C*03:05, and HLA-C*14:03 allele. In some embodiments, the HLA serotype is HLA-A*02 and/or the HLA allele is HLA-A*02:01 allele. In some embodiments, the binding proteins provided herein are genetically engineered, isolated, and/or purified.

In some embodiments, the binding proteins have a higher binding affinity to the PRAME peptide-MHC (pMHC) than does a known T-cell receptor (e.g., a comparator TCR described herein). For example, the binding proteins may have at least 1.2 fold, 1.5 fold, 1.8 fold, 2.0 fold, 2.2 fold, 2.5 fold, 2.8 fold, 3 fold, 3.5 fold, 4 fold, 4.5 fold, 5 fold, 5.5 fold, 6 fold, 6.5 fold, 7 fold, 7.5 fold, 8 fold, 8.5 fold, 9 fold, 9.5 fold, 10 fold, 11 fold, 12 fold, 13 fold, 14 fold, 15 fold, 16 fold, 17 fold, 18 fold, 19 fold, 20 fold, 25 fold, 30 fold, 35 fold, 40 fold, 45 fold, 50 fold, 60 fold, 70 fold, 80 fold, 90 fold, 100 fold, 1000 fold, 5000 fold, 10000 fold, 50000 fold, 100000 fold, 500000 fold, 1000000 fold, or more, or any range in between, inclusive, such as 1.2 fold to 2 fold, higher binding affinity to the PRAME peptide-MHC (pMHC) than does a known T-cell receptor (e.g., a comparator TCR described herein).

In some embodiments, the binding protein induces higher T cell expansion, cytokine release, and/or cytotoxic killing than does a known T-cell receptor (e.g., a comparator TCR described herein) when contacted with target cells with expression of PRAME at a certain level or below. For example, in some embodiments of any aspect described herein, PRAME level can be expressed in terms of transcripts per million and may be, for example, less than or equal to about 1,000 transcript per million transcripts (TPM), 950 TPM, 900 TPM, 850 TPM, 800 TPM, 750 TPM, 700 TPM, 650 TPM, 600 TPM, 550 TPM, 500 TPM, 450 TPM, 400 TPM, 350 TPM, 300 TPM, 250 TPM, 200 TPM, 150 TPM, 100 TPM, 95 TPM, 90 TPM, 85 TPM, 80 TPM, 75 TPM, 70 TPM, 65 TPM, 60 TPM, 55 TPM, 50 TPM, 45 TPM, 40 TPM, 35 TPM, 34 TPM, 33 TPM, 32 TPM, 31 TPM, 30 TPM, 29 TPM, 28 TPM, 27 TPM. 26 TPM, 25 TPM, 24 TPM, 23 TPM, 22 TPM, 21 TPM, 20 TPM, 19 TPM, 18 TPM, 17 TPM, 16 TPM, 15 TPM, 14 TPM, 13 TPM, 12 TPM, 11 TPM, 10 TPM, 9 TPM, 8 TPM, 7 TPM, 6 TPM, 5 TPM, 4 TPM, 3 TPM, 2 TPM, and 1 TPM, or any range in between, inclusive, such as less than or equal to about 1,000 TPM to less than or equal to about 35 TPM). In some embodiments, the low PRAME expression level is termed “heterozygous expression” meaning between about 1 TPM and about 35 TPM, or any range in between, inclusive, such as 32 TPM or 1-32 TPM. A higher expression is 36 TPM and higher. As described further herein. TPM is measured according to well-known techniques, such as RNA-Seq, and gene expression TPM data are well known in the art for a variety of cell lines, tissue types, and the like (see, for example, the Broad Institute Cancer Cell Line Encyclopedia (CCLE) on the World Wide Web at portals.broadinstitute.org). In some embodiments, the binding protein induces at least 1.2 fold, 1.5 fold, 1.8 fold, 2.0 fold, 2.2 fold, 2.5 fold, 2.8 fold, 3 fold, 3.5 fold, 4 fold, 4.5 fold, 5 fold, 5.5 fold, 6 fold, 6.5 fold, 7 fold, 7.5 fold, 8 fold, 8.5 fold, 9 fold, 9.5 fold, 10 fold, 11 fold, 12 fold, 13 fold, 14 fold, 15 fold, 16 fold, 17 fold, 18 fold, 19 fold, 20 fold, 25 fold, 30 fold, 35 fold, 40 fold, 45 fold, 50 fold, 60 fold, 70 fold, 80 fold, 90 fold, 100 fold, 1000 fold, or more, or any range in between, inclusive, such as 1.2 fold to 2 fold, increase in T cell expansion, cytokine release, and/or cytotoxic killing than does a known T-cell receptor (e.g., a comparator TCR described herein) when contacted with target cells expressing PRAME peptide epitope, such as with heterozygous expression of PRAME peptide epitope.

In some embodiments, the expression of PRAME is detected using RNA-sequencing (RNA-seq). RNA-seq generally comprises the following steps: obtaining a sample containing genetic material, isolating total RNA from the sample obtained, preparing an amplified cDNA library from the total RNA, sequencing the amplified cDNA library, and analyzing and profiling the amplified cDNA to assess the expression level of different transcripts. The sample can be a population of cells, a tissue sample, a biopsy sample, a cell culture, or a single cell. Total RNA can be isolated from the biological sample using any method known in the art. In certain embodiments, total RNA is extracted from plasma. Plasma RNA extraction is described in Enders et al., “The Concentration of Circulating Corticotropin-Releasing Homer mRNA in Material Plasma Is Inclined in Preeclampsia,” Clinr. As described therein, the plasma collected after the centrifugation step is mixed with Trizol LS reagent (Invitrogen) and chloroform. The mixture is centrifuged and the aqueous layer is transferred to a new tube. Ethanol is added to this aqueous layer. The mixture is then placed in an RNeasy mini column (Qiagen) and processed according to the manufacturer's recommendations.

In some embodiments, RNA-seq described herein includes the step of preparing amplified cDNA from total RNA. For example, cDNA is prepared and the isolated RNA sample is randomly amplified without dilution, or the mixture of genetic material in the isolated RNA is dispersed into individual reaction samples. In certain embodiments, amplification is initiated randomly at the 3′ end and throughout the entire transcriptome in the sample to amplify both mRNA and non-polyadenylated transcripts. In this way, double-stranded cDNA amplification products are optimized for the generation of sequencing libraries for next generation sequencing platforms. A kit suitable for amplification of cDNA by the method encompassed by the present invention includes, for example, Ovation® RNA-Seq System.

In some embodiments, RNA-seq described herein includes the step of sequencing the amplified cDNA. Any known sequencing method can be used to sequence the amplified cDNA mixture including the single molecule sequencing method. In certain embodiments, the amplified cDNA is sequenced by whole transcriptome shotgun sequencing. Whole transcriptome shotgun sequencing can be performed using various next generation sequencing platforms such as Illumina® Genome Analyzer platform, ABI SOLiD™ Sequencing platform, or Life Science's 454 Sequencing platform.

In some embodiments. RNA-seq described herein further comprises performing digital counting and analysis on the cDNA. The number of amplified sequences for each transcript in the amplified sample can be quantified by sequence reading (one reading per amplified strand). In some embodiments, transcript per million (TPM) is used to quantify the expression level of a particular transcript. TPM may be calculated as shown in Wagner et al. (2012) Theory in Biosciences 131:281-285, the content of which is incorporated by reference herein in its entirety.

In certain embodiments, the binding proteins recognize a PRAME immunogenic peptide in a complex with MHC molecules, such as particular HLA molecules having particular HLA alpha chain alleles. For example, binding proteins listed in Table 2A were identified as binders of PRAME immunogenic peptides in association with an MHC whose alpha chain had an HLA-A*02 serotype, such as that encoded by an HLA-A*02:01 allele, as described further in the Examples section. In some embodiments, the binding proteins recognize a complex of PRAME immunogenic peptide and an MHC molecule, wherein the MHC molecule comprises an MHC alpha chain that is an HLA serotype selected from the group consisting of HLA-A*02, HLA-A*03, HLA-A*O1, HLA-A*11, HLA-A*24, HLA-B*07, HLA-C*07, HLA-C*01, HLA-C*02, HLA-C*03, HLA-C*04, HLA-C*05, HLA-C*06, HLA-C*08, HLA-C*12, HLA-C*14, HLA-C*15, HLA-C*16, HLA-C*17, and HLA-C*18, optionally wherein the HLA allele is selected from the group consisting of HLA-A*02:01, HLA-A*02:02, HLA-A*02:03, HLA-A*02:04, HLA-A*02:05, HLA-A*02:06, HLA-A*02:07, HLA-A*02:10, HLA-A*02:11, HLA-A*02:12, HLA-A*02:13, HLA-A*02:14, HLA-A*02:16, HLA-A*02:17, HLA-A*02:19, HLA-A*02:20, HLA-A*02:22, HLA-A*02:24, HLA-A*02:30, HLA-A*02:42, HLA-A*02:53, HLA-A*02:60, HLA-A*02:74 allele, HLA-A*03:01, HLA-A*03:02, HLA-A*03:05, HLA-A*03:07, HLA-A*01:01, HLA-A*01:02, HLA-A*01:03, HLA-A*01:16 allele, HLA-A*11:01, HLA-A*11:02, HLA-A*l 1:03, HLA-A*11:04, HLA-A*l 1:05, HLA-A*11:19 allele, HLA-A*24:02, HLA-A*24:03, HLA-A*24:05, HLA-A*24:07, HLA-A*24:08, HLA-A*24:10, HLA-A*24:14, HLA-A*24:17, HLA-A*24:20, HLA-A*24:22, HLA-A*24:25, HLA-A*24:26, HLA-A*24:58 allele, HLA-B*07:02, HLA-B*07:04, HLA-B*07:05, HLA-B*07:09, HLA-B*07:10, HLA-B*07:15, HLA-B*07:21, HLA-C*07:02, HLA-C*07:01, HLA-C*04:01, HLA-C*06:02, HLA-C*03:04, HLA-C*05:01, HLA-C*16:01, HLA-C*02:02, HLA-C*03:03, HLA-C*12:03, HLA-C*08:02, HLA-C*01:02, HLA-C*17:01, HLA-C*15:02, HLA-C*14:02, HLA-C*12:02, HLA-C*07:04, HLA-C*08:01, HLA-C*03:02, HLA-C*18:01, HLA-C*15:05, HLA-C*16:02, HLA-C*08:04, HLA-C*03:05, and HLA-C*14:03 allele. In some embodiments, the PRAME immunogenic peptides are derived from a human PRAME protein and/or a PRAME protein shown in Table 3. In some embodiments, one or more PRAME immunogenic peptides are administered alone or in combination with an adjuvant.

In some embodiments, the binding proteins do not bind to a peptide-MHC (pMHC) complex, optionally wherein the peptide is derived from an “off-target” described herein, such as PLA2G4E. EFNA1, and/or SLC26A1.

In some embodiments, the binding protein does not bind to a an “off-target”-peptide-MHC (pMHC) complex, such as PLA2G4E, EFNA1, and/or SLC26A1-peptide-MHC (pMHC) complex.

In some embodiments, the binding proteins provided herein include (e.g., comprise, consist essentially of, or consist of): a) a TCR alpha chain sequence with at least about 80%, 81%, 82%, 83%, 84%, 85%, 86%, 87%, 88%, 89%, 90%, 91%, 92%, 93%, 94%, 95%, 96%, 97%, 98%, 99%, or more identity to a TCR alpha chain sequence selected from the group consisting of the TCR alpha sequences listed in Table 2; and/or b) a TCR beta chain sequence with at least about 80%, 81%, 82%, 83%, 84%, 85%, 86%, 87%, 88%, 89%, 90%, 91%, 92%, 93%, 94%, 95%, 96%, 97%, 98%, 99%, or more identity to a TCR beta chain sequence selected from the group consisting of the TCR beta chain sequences listed in Table 2.

In some embodiments, the binding proteins provided herein include (e.g., comprise, consist essentially of, or consist of): a) a TCR alpha chain sequence selected from the group consisting of the TCR alpha chain sequences listed in Table 2; and/or b) a TCR beta chain sequence selected from the group consisting of the TCR beta chain sequences listed in Table 2.

In some embodiments, the binding proteins provided herein include (e.g., comprise, consist essentially of, or consist of): a) a TCR alpha chain variable (V_{α}) domain sequence with at least about 80%, 81%, 82%, 83%, 84%, 85%, 86%, 87%, 88%, 89%, 90%, 91%, 92%, 93%, 94%, 95%, 96%, 97%, 98%, 99%, or more identity to a TCR alpha chain variable (V_{α}) domain sequence selected from the group consisting of the TCR V_{α} domain sequences listed in Table 2; and/or b) a TCR beta chain variable (V_{β}) domain sequence with at least about 80%, 81%, 82%, 83%, 84%, 85%, 86%, 87%, 88%, 89%, 90%, 91%, 92%, 93%, 94%, 95%, 96%, 97%, 98%, 99%, or more identity to a TCR beta chain variable (V_{β}) domain sequence selected from the group consisting of the TCR V_{β} domain sequences listed in Table 2.

In some embodiments, the binding proteins provided herein include (e.g., comprise, consist essentially of, or consist of): a) a TCR alpha chain variable (V_{α}) domain sequence selected from the group consisting of the TCR V_{α} domain sequences listed in Table 2; and/or b) a TCR beta chain variable (V_{β}) domain sequence selected from the group consisting of the TCR V_{β} domain sequences listed in Table 2.

In some embodiments, the binding proteins provided herein include (e.g., comprise, consist essentially of, or consist of at least one (e.g., one, two or three, such as CDR3 alone or in combination with a CDR1 and CDR2)) TCR alpha chain complementarity determining region (CDR) sequence with at least about 80%, 81%, 82%, 83%, 84%, 85%, 86%, 87%, 88%, 89%, 90%, 91%, 92%, 93%, 94%, 95%, 96%, 97%, 98%, 99%, or more identity to a TCR alpha chain CDR sequence selected from the group consisting of the TCR alpha chain CDR sequences listed in Table 2, CDR3 is believed to be the main CDR responsible for recognizing processed antigen and CDR1 and CDR2 mainly interact with the MHC, so, in some embodiments, binding protein comprising a CDR3 alone from a TCR alpha chain and/or a CDR3 alone from a TCR beta chain listed in Table 2, each CDR3 having a sequence homology as recited in this paragraph, are provided.

In some embodiments, the binding proteins provided herein may also include (e.g., comprise, consist essentially of, or consist of at least one (e.g., one, two or three, such as CDR3 alone or in combination with a CDR1 and CDR2)) TCR beta chain complementarity determining region (CDR) sequence with at least about 80%, 81%, 82%, 83%, 84%, 85%, 86%, 87%, 88%, 89%, 90%, 91%, 92%, 93%, 94%, 95%, 96%, 97%, 98%, 99%, or more identity to a TCR beta chain CDR sequence selected from the group consisting of the TCR beta chain CDR sequences listed in Table 2. As described above, CDR3 is believed to be the main CDR responsible for recognizing processed antigen and CDR1 and CDR2 mainly interact with the MHC, so, in some embodiments, binding protein comprising a CDR3 alone from a TCR beta chain and/or a CDR3 alone from a TCR alpha chain listed in Table 2, each CDR3 having a sequence homology as recited in this paragraph, are provided.

In some embodiments, the binding proteins provided herein include (e.g., comprise, consist essentially of, or consist of at least one (e.g., one, two or three)) TCR alpha chain complementarity determining region (CDR) listed in Table 2.

In some embodiments, the binding proteins provided herein may also include (e.g., comprise, consist essentially of, or consist of at least one (e.g., one, two or three)) TCR beta chain complementarity determining region (CDR) listed in Table 2.

In some embodiments, the binding proteins provided herein include (e.g., comprise, consist essentially of, or consist of) a TCR alpha chain constant region (Ca) sequence with at least about 80%, 81%, 82%, 83%, 84%, 85%, 86%, 87%, 88%, 89%, 90%, 91%, 92%, 93%, 94%, 95%, 96%, 97%, 98%, 99%, or more identity to a TCR Cα sequence listed in Table 2.

In some embodiments, the binding proteins provided herein may also include (e.g., comprise, consist essentially of, or consist of) a TCR beta chain constant region (C_{β}) sequence with at least about 80%, 81%, 82%, 83%, 84%, 85%, 86%, 87%, 88%, 89%, 90%, 91%, 92%, 93%, 94%, 95%, 96%, 97%, 98%, 99%, or more identity to a TCR C_{β} sequence listed in Table 2.

In some embodiments, the binding proteins provided herein include (e.g., comprise, consist essentially of, or consist of) a TCR alpha chain constant region (C_{α}) sequence selected from the group consisting of the TCR C_{α} sequences listed in Table 2.

In some embodiments, the binding proteins provided herein may also include (e.g., comprise, consist essentially of, or consist of) a TCR beta chain constant region (C_{β}) sequence selected from the group consisting of the TCR CP sequences listed in Table 2.

TABLE 2
TCR sequences recognizing a PRAME antigen
Table 2A
TCR sequences recognizing a PRAME antigen presented by HLA serotype HLA-A*02

PRAME-425-366 WT sequence
Alpha chain:
TRAV38-2DV8/TRAJ50/TRAC
Alpha chain DNA sequence
ATGGCCTGTCCTGGCTTCCTGTGGGCCCTTGTGATCAGCACTTGCCTGGAATTCA
GCATGGCTCAGACAGTCACCCAGTCTCAGCCCGAAATGAGCGTCCAAGAGGCTG
AAACCGTGACTCTGTCTTGTACCTACGACACCTCCGAGAGCGATTACTACCTCT
TTTGGTATAAGCAACCGCCGTCCAGGCAAATGATCCTCGTGATCCGGCAAGAAG
CTTACAAACAGCAGAATGCTACCGAAAACCGGTTCTCCGTCAATTTTCAGAAAG
CCGCTAAGAGCTTTAGCCTGAAAATCTCCGACTCTCAGCTCGGCGACGCTGCTAT
GTATTTCTGTGCCTACCGCAAAACTTCTTACGATAAAGTCATTTTTGGGCCAG
GGACAAGCTTATCAGTCATTCCAAatatccagaaccctgaccctgccgtgtaccagctgagagactctaaatcc
agtgacaagtctgtctgcctattcaccgattttgattctcaaacaaatgtgtcacaaagtaaggattctgatgtgtatatcacagacaaaact
gtgctagacatgaggtctatggacttcaagagcaacagtgctgtggcctggagcaacaaatctgactttgcatgtgcaaacgccttcaa
caacagcattattccagaagacaccttcttccccagcccagaaagttcctgtgatgtcaagctggtcgagaaaagctttgaaacagatac
gaacctaaactttcaaaacctgtcagtgattgggttccgaatcctcctcctgaaagtggccgggtttaatctgctcatgacgctgcggctg
tggtccagc
Alpha chain protein sequence
MACPGFLWALVISTCLEFSMAQTVTQSQPEMSVQEAETVTLSCTYDTSESDYYLFW
YKQPPSRQMILVIRQEAYKQQNATENRFSVNFQKAAKSFSLKISDSQLGDAAMYFC
AYRKTSYDKVIFGPGTSLSVIPNiqnpdpavyqlrdskssdksvclftdfdsqtnvsqskdsdvyitdktvldmr
smdfksnsavawsnksdfacanafnnsiipedtffpspesscdvklveksfetdtnlnfqnlsvigfrilllkvagfnllmtlrlwss
Beta chain:
TRBV13/TRBJ2-1/TRBC1
Beta chain DNA sequence
ATGCTGAGCCCCGACCTGCCTGACAGCGCTTGGAATACCAGACTCCTGTGCAGA
GTGATGCTGTGCCTGCTTGGAGCTGGAAGTGTGGCTGCTGGTGTCATTCAGTCCC
CAAGGCACCTGATCAAAGAGAAGAGAGAGACAGCCACTCTGAAGTGCTACCCCA
TTCCTAGACACGACACGGTCTATTGGTATCAGCAAGGACCTGGACAGGACCCTC
AGTTCCTGATCAGCTTCTACGAGAAGATGCAGAGCGACAAGGGCAGCATCCCC
GACAGATTTTCTGCCCAGCAGTTCAGCGACTACCACAGCGAGCTGAACATGAGC
AGCCTGGAACTGGGCGATAGCGCCCTGTACTTCTGTGCCTCTTCTTTCGCACGC
CTGGAAGGTCGCGATAATGAACAATTTTTTGGGCCAGGGACACGGCTCACCGT
GCTAGaggacctgaacaaggtgttcccacccgaggtcgctgtgtttgagccatcagaagcagagatctcccacacccaaaaggc
cacactggtgtgcctggccacaggcttcttccctgaccacgtggagctgagctggtgggtgaatgggaaggaggtgcacagtggggt
cagcacggacccgcagcccctcaaggagcagcccgccctcaatgactccagatactgcctgagcagccgcctgagggtctcggcc
accttctggcagaacccccgcaaccacttccgctgtcaagtccagttctacgggctctcggagaatgacgagtggacccaggatagg
gccaaacccgtcacccagatcgtcagcgccgaggcctggggtagagcagactgtggctttacctcggtgtcctaccagcaaggggt
cctgtctgccaccatcctctatgagatcctgctagggaaggccaccctgtatgctgtgctggtcagcgcccttgtgttgatggccatggt
caagagaaaggatttc
Beta chain protein sequence
MLSPDLPDSAWNTRLLCRVMLCLLGAGSVAAGVIQSPRHLIKEKRETATLKCYPIPR
HDTVYWYQQGPGQDPQFLISFYEKMQSDKGSIPDRFSAQQFSDYHSELNMSSLELG
DSALYFCASSFARLEGRDNEQFFGPGTRLTVLEdlnkvfppevavfepseaeishtqkatlvclatgffp
dhvelswwvngkevhsgvstdpqplkeqpalndsryclssrlrvsatfwqnprnhfrcqvqfyglsendewtqdrakpvtqivs
aeawgradcgftsvsyqqgvlsatilyeillgkatlyavlvsalvlmamvkrkdf
PRAME-425-366 MGTM codon optimized sequence (also known as “366”, “TCR 366”,
TCR expressed by “TSC-203-A02”, and TCR expressed by “TSC-203-A0201”)
Alpha chain:
TRAV38-2DV8/TRAJ50/MGTM modified TRAC
Alpha chain DNA sequence
ATGGCCTGTCCTGGCTTCCTGTGGGCCCTTGTGATCAGCACTTGCCTGGAATTCA
GCATGGCTCAGACAGTCACCCAGTCTCAGCCCGAAATGAGCGTCCAAGAGGCTG
AAACCGTGACTCTGTCTTGTACCTACGACACCTCCGAGAGCGATTACTACCTCT
TTTGGTATAAGCAACCGCCGTCCAGGCAAATGATCCTCGTGATCCGGCAAGAAG
CTTACAAACAGCAGAATGCTACCGAAAACCGGTTCTCCGTCAATTTTCAGAAAG
CCGCTAAGAGCTTTAGCCTGAAAATCTCCGACTCTCAGCTCGGCGACGCTGCTAT
GTATTTCTGTGCCTACCGCAAAACTTCTTACGATAAAGTCATTTTTGGGCCAG
GGACAAGCTTATCAGTCATTCCAAacatccagaaccccgaccccgccgtgtaccagctgagggactccaagt
ccagcgacaagagcgtgtgtctgtttacggacttcgacagccagaccaacgtgagtcaaagcaaggacagcgacgtctacataacg
gataagaccgtgctggacatgcggagcatggacttcaagagcaacagcgccgtggcctggtccaacaagagcgacttcgcctgcgc
caacgccttcaacaacagcatcatccccgaggacaccttcttccccagcagcgacgtgccctgcgacgtgaaactggtggagaagtc
cttcgagacagacaccaatctgaactttcagaacctgctggtgatcgtgctgcggattctgctgctgaaagtggccggcttcaatctgct
gatgaccctgcggctgtggagcagc
Alpha chain protein sequence
MACPGFLWALVISTCLEFSMAQTVTQSQPEMSVQEAETVTLSCTYDTSESDYYLFW
YKQPPSRQMILVIRQEAYKQQNATENRFSVNFQKAAKSFSLKISDSQLGDAAMYFC
AYRKTSYDKVIFGPGTSLSVIPNiqnpdpavyqlrdskssdksvclftdfdsqtnvsqskdsdvyitdktvldmr
smdfksnsavawsnksdfacanafnnsiipedtffpssdvpcdvklveksfetdtnlnfqnllvivlrilllkvagfnllmtlrlwss
Beta chain:
TRBV13/TRBJ2-1/MGTM modified TRBC
Beta chain DNA sequence
ATGCTGAGCCCCGACCTGCCTGACAGCGCTTGGAATACCAGACTCCTGTGCAGA
GTGATGCTGTGCCTGCTTGGAGCTGGAAGTGTGGCTGCTGGTGTCATTCAGTCCC
CAAGGCACCTGATCAAAGAGAAGAGAGAGACAGCCACTCTGAAGTGCTACCCCA
TTCCTAGACACGACACGGTCTATTGGTATCAGCAAGGACCTGGACAGGACCCTC
AGTTCCTGATCAGCTTCTACGAGAAGATGCAGAGCGACAAGGGCAGCATCCCC
GACAGATTTTCTGCCCAGCAGTTCAGCGACTACCACAGCGAGCTGAACATGAGC
AGCCTGGAACTGGGCGATAGCGCCCTGTACTTCTGTGCCTCTTCTTTCGCACGC
CTGGAAGGTCGCGATAATGAACAATTTTTTGGGCCAGGGACACGGCTCACCGT
GCTAGaagatctgaacaaggtgttccctccagaggtggccgtgttcgagccttctaaggccgagatcgcccacacacaaaaagc
caccctcgtgtgcctggccaccggctttttccccgaccacgtggaactgtcttggtgggtcaacggcaaagaggtgcactccggcgtg
tcaacggatccccagcctctgaaagaacagcctgccctgaacgacagccggtactgcctgagctccagactgagagtgtccgccac
cttctggcagaacccccggaaccacttcagatgccaggtgcagttttacggcctgagcgagaacgacgagtggacccaggacagag
ccaagcccgtgacacaaatcgtgtctgccgaagcctggggaagagccgattgcggcatcaccagcgcctcctatcaccagggcgtg
ctgagcgccacaatcctgtacgaaatcctgctgggcaaggccaccctgtacgccgtgctggtgtctgctctggtgctgatggccatggt
caagcggaaggactttggcagcggc
Beta chain protein sequence
MLSPDLPDSAWNTRLLCRVMLCLLGAGSVAAGVIQSPRHLIKEKRETATLKCYPIPR
HDTVYWYQQGPGQDPQFLISFYEKMQSDKGSIPDRFSAQQFSDYHSELNMSSLELG
DSALYFCASSFARLEGRDNEQFFGPGTRLTVLEdlnkvfppevavfepskaeiahtqkatlvclatgffp
dhvelswwvngkevhsgvstdpqplkeqpalndsryclssrlrvsatfwqnprnhfrcqvqfyglsendewtqdrakpvtqivs
aeawgradcgitsasyhqgvlsatilyeillgkatlyavlvsalvlmamvkrkdfgsg
Complete Beta and Alpha ORF DNA Sequence (The underlined italic region in the “Furin-
P2A” site encodes a sequence allowing for expression of two polypeptide chains in a single
cassette)
ATGCTGAGCCCCGACCTGCCTGACAGCGCTTGGAATACCAGACTCCTGTGCAGA
GTGATGCTGTGCCTGCTTGGAGCTGGAAGTGTGGCTGCTGGTGTCATTCAGTCCC
CAAGGCACCTGATCAAAGAGAAGAGAGAGACAGCCACTCTGAAGTGCTACCCCA
TTCCTAGACACGACACGGTCTATTGGTATCAGCAAGGACCTGGACAGGACCCTC
AGTTCCTGATCAGCTTCTACGAGAAGATGCAGAGCGACAAGGGCAGCATCCCC
GACAGATTTTCTGCCCAGCAGTTCAGCGACTACCACAGCGAGCTGAACATGAGC
AGCCTGGAACTGGGCGATAGCGCCCTGTACTTCTGTGCCTCTTCTTTCGCACGC
CTGGAAGGTCGCGATAATGAACAATTTTTTGGGCCAGGGACACGGCTCACCGT
GCTAGaagatctgaacaaggtgttccctccagaggtggccgtgttcgagccttctaaggccgagatcgcccacacacaaaaagc
caccctcgtgtgcctggccaccggctttttccccgaccacgtggaactgtcttggtgggtcaacggcaaagaggtgcactccggcgtg
tcaacggatccccagcctctgaaagaacagcctgccctgaacgacagccggtactgcctgagctccagactgagagtgtccgccac
cttctggcagaacccccggaaccacttcagatgccaggtgcagttttacggcctgagcgagaacgacgagtggacccaggacagag
ccaagcccgtgacacaaatcgtgtctgccgaagcctggggaagagccgattgcggcatcaccagcgcctcctatcaccaggggtg
ctgagcgccacaatcctgtacgaaatcctgctgggcaaggccaccctgtacgccgtgctggtgtctgctctggtgctgatggccatggt
caagcggaaggactttggcagcggcagagccaaaaggtccgggagcggtGCGACAAACTTTAGCCTGTTGAAA
CAAGCCGGCGACGTTGAAGAGAACCCCGGACCTATGGCCTGTCCTGGCTTCCTGTG
GGCCCTTGTGATCAGCACTTGCCTGGAATTCAGCATGGCTCAGACAGTCACCCAG
TCTCAGCCCGAAATGAGCGTCCAAGAGGCTGAAACCGTGACTCTGTCTTGTACCT
ACGACACCTCCGAGAGCGATTACTACCTCTTTTGGTATAAGCAACCGCCGTCCA
GGCAAATGATCCTCGTGATCCGGCAAGAAGCTTACAAACAGCAGAATGCTACC
GAAAACCGGTTCTCCGTCAATTTTCAGAAAGCCGCTAAGAGCTTTAGCCTGAAAA
TCTCCGACTCTCAGCTCGGCGACGCTGCTATGTATTTCTGTGCCTACCGCAAAA
CTTCTTACGATAAAGTCATTTTTGGGCCAGGGACAAGCTTATCAGTCATTCCAA
acatccagaaccccgaccccgccgtgtaccagctgagggactccaagtccagcgacaagagcgtgtgtctgtttacggacttcgaca
gccagaccaacgtgagtcaaagcaaggacagcgacgtctacataacggataagaccgtgctggacatgcggagcatggacttcaag
agcaacagcgccgtggcctggtccaacaagagcgacttcgcctgcgccaacgccttcaacaacagcatcatccccgaggacacctt
cttccccagcagcgacgtgccctgcgacgtgaaactggtggagaagtccttcgagacagacaccaatctgaactttcagaacctgctg
gtgatcgtgctgcggattctgctgctgaaagtggccggcttcaatctgctgatgaccctgcggctgtggagcagc
Complete Beta and Alpha ORF Protein Sequence (The underlined italic region in the “Furin-
P2A” site allows expression of two polypeptide chains in a single cassette)
MLSPDLPDSAWNTRLLCRVMLCLLGAGSVAAGVIQSPRHLIKEKRETATLKCYPIPR
HDTVYWYQQGPGQDPQFLISFYEKMQSDKGSIPDRFSAQQFSDYHSELNMSSLELG
DSALYFCASSFARLEGRDNEQFFGPGTRLTVLEdlnkvfppevavfepskaeiahtqkatlvclatgffp
dhvelswwvngkevhsgvstdpqplkeqpalndsryclssrlrvsatfwqnprnhfrcqvqfyglsendewtqdrakpvtqivs
aeawgradcgitsasyhqgvlsatilyeillgkatlyavlvsalvlmamvkrkdfgsgrakrsgsgATNFSLLKQAGDVE
ENPGPMACPGFLWALVISTCLEFSMAQTVTQSQPEMSVQEAETVTLSCTYDTSESDY
YLFWYKQPPSRQMILVIRQEAYKQQNATENRFSVNFQKAAKSFSLKISDSQLGDAA
MYFCAYRKTSYDKVIFGPGTSLSVIPNiqnpdpavyqlrdskssdksvclftdfdsqtnvsqskdsdvyitd
ktvldmrsmdfksnsavawsnksdfacanafnnsiipedtffpssdvpcdvklveksfetdtnlnfqnllvivlrillikvagfnllm
tlrlwss
PRAME-425-358 WT sequence
Alpha chain:
TRAV30/TRAJ20/TRAC
Alpha chain DNA sequence
ATGGAAACCCTGCTGAAGGTGCTGTCTGGCACCCTGCTGTGGCAGCTGACATGG
GTCCGATCTCAGCAGCCTGTGCAGTCTCCTCAGGCCGTGATTCTGAGAGAAGGCG
AGGACGCCGTGATCAACTGCAGCAGCTCTAAGGCCCTGTACAGCGTGCACTGG
TACAGGCAGAAACACGGCGAGGCCCCAGTGTTTCTGATGATTCTGCTGAAAGGC
GGCGAGCAGAAGGGCCACGATAAGATCTCCGCCAGCTTCAACGAGAAGAAGCA
GCAGTCCAGCCTGTACCTGACAGCCAGCCAGCTGAGCTACAGCGGCACCTATTTC
TGTGGCACAGAAGGTACTGGTGACTACAAGCTCTCTTTTGGAGCCGGAACCA
CAGTAACTGTAAGAGCAAatatccagaaccctgaccctgccgtgtaccagctgagagactctaaatccagtgacaag
tctgtctgcctattcaccgattttgattctcaaacaaatgtgtcacaaagtaaggattctgatgtgtatatcacagacaaaactgtgctaga
catgaggtctatggacttcaagagcaacagtgctgtggcctggagcaacaaatctgactttgcatgtgcaaacgccttcaacaacagcat
tattccagaagacaccttcttccccagcccagaaagttcctgtgatgtcaagctggtcgagaaaagctttgaaacagatacgaacctaaa
ctttcaaaacctgtcagtgattgggttccgaatcctcctcctgaaagtggccgggtttaatctgctcatgacgctgcggctgtggtccagc
Alpha chain protein sequence
METLLKVLSGTLLWQLTWVRSQQPVQSPQAVILREGEDAVINCSSSKALYSVHWYR
QKHGEAPVFLMILLKGGEQKGHDKISASFNEKKQQSSLYLTASQLSYSGTYFCGTE
GTGDYKLSFGAGTTVTVRANiqnpdpavyqlrdskssdksvclftdfdsqtnvsqskdsdvyitdktvldmrs
mdfksnsavawsnksdfacanafnnsiipedtffpspesscdvklveksfetdtnlnfqnlsvigfrilllkvagfnllmtlrlwss
Beta chain:
TRBV27/TRBJ2-7/TRBC1
Beta chain DNA sequence
ATGGGACCTCAGCTGCTGGGATATGTGGTGCTGTGTCTGCTCGGAGCTGGACCCC
TGGAAGCTCAAGTGACACAGAACCCCAGATACCTGATCACCGTGACCGGCAAAA
AGCTGACCGTGACCTGTAGCCAGAACATGAACCACGAGTACATGAGCTGGTAT
CGGCAAGACCCTGGCCTGGGGCTGAGACAGATCTACTATAGCATGAACGTGGA
AGTGACCGACAAAGGCGACGTGCCCGAGGGCTATAAGGTGTCCCGGAAAGAGA
AGCGGAACTTTCCACTGATCCTGGAATCCCCATCTCCTAACCAGACCAGCCTGTA
TTTTTGCGCTAGTTCTGCCGGGACCGGGGGGCATGAGCAATACTTCGGGCCG
GGCACCAGGCTCACGGTCACAGaggacctgaacaaggtgttcccacccgaggtcgctgtgtttgagccatcaga
agcagagatctcccacacccaaaaggccacactggtgtgcctggccacaggcttcttccctgaccacgtggagctgagctggtgggt
gaatgggaaggaggtgcacagtggggtcagcacggacccgcagcccctcaaggagcagcccgccctccaatgactccagatactgc
ctgagcagccgcctgagggtctcggccaccttctggcagaacccccgcaaccacttccgctgtcaagtccagttctacgggctctcgg
agaatgacgagtggacccaggatagggccaaacccgtcacccagatcgtcagcgccgaggcctggggtagagcagactgtggctt
tacctcggtgtcctaccagcaaggggtcctgtctgccaccatcctctatgagatcctgctagggaaggccaccctgtatgctgtgctggt
cagcgcccttgtgttgatggccatggtcaagagaaaggatttc
Beta chain protein sequence
MGPQLLGYVVLCLLGAGPLEAQVTQNPRYLITVTGKKLTVTCSQNMNHEYMSWYR
QDPGLGLRQIYYSMNVEVTDKGDVPEGYKVSRKEKRNFPLILESPSPNQTSLYFCAS
SAGTGGHEQYFGPGTRLTVTEdknkvfppevavfepseaeishtqkatlvclatgffpdhvelswwvngkevh
sgvstdpqplkeqpalndsryclssrlrvsatfwqnprnhfrcqvqfyglsendewtqdrakpvtqivsaeawgradegftsvsyq
qgvlsatilyeillgkatlyavlvsalvlmamvkrkdf
PRAME-425-358 MGTM codon optimized sequence (also known as “358” or TCR 358”)
Alpha chain:
TRAV30/TRAJ20/MGTM modified TRAC
Alpha chain DNA sequence
ATGGAAACCCTGCTGAAGGTGCTGTCTGGCACCCTGCTGTGGCAGCTGACATGG
GTCCGATCTCAGCAGCCTGTGCAGTCTCCTCAGGCCGTGATTCTGAGAGAAGGCG
AGGACGCCGTGATCAACTGCAGCAGCTCTAAGGCCCTGTACAGCGTGCACTGG
TACAGGCAGAAACACGGCGAGGCCCCAGTGTTTCTGATGATTCTGCTGAAAGGC
GGCGAGCAGAAGGGCCACGATAAGATCTCCGCCAGCTTCAACGAGAAGAAGCA
GCAGTCCAGCCTGTACCTGACAGCCAGCCAGCTGAGCTACAGCGGCACCTATTTC
TGTGGCACAGAAGGTACTGGTGACTACAAGCTCTCTTTTGGAGCCGGAACCA
CAGTAACTGTAAGAGCAAacatccagaaccccgaccccgccgtgtaccagctgagggactccaagtccagcgaca
agagcgtgtgtctgtttacggacttcgacagccagaccaacgtgagtcaaagcaaggacagcgacgtctacataacggataagaccg
tgctggacatgcggagcatggacttcaagagcaacagcgccgtggcctggtccaacaagagcgacttcgcctgcgccaacgccttc
aacaacagcatcatccccgaggacaccttcttccccagcagcgacgtgccctgcgacgtgaaactggtggagaagtccttcgagaca
gacaccaatctgaactttcagaacctgctggtgatcgtgctgcggattctgctgctgaaagtggccggcttcaatctgctgatgaccctg
cggctgtggagc
Alpha chain protein sequence
METLLKVLSGTLLWQLTWVRSQQPVQSPQAVILREGEDAVINCSSSKALYSVHWYR
QKHGEAPVFLMILLKGGEQKGHDKISASFNEKKQQSSLYLTASQLSYSGTYFCGTE
GTGDYKLSFGAGTTVTVRANiqnpdpavyqlrdskssdksvclftdfdsqtnvsqskdsdvyitdktvldmrs
mdfksnsavawsnksdfacanafnnsiipedtffpssdvpcdvklveksfetdtnlnfqnllvivlrillikvagfnllmtlrlws
Beta chain:
TRBV27/TRBJ2-7/MGTM modified TRBC
Beta chain DNA sequence
ATGGGACCTCAGCTGCTGGGATATGTGGTGCTGTGTCTGCTCGGAGCTGGACCCC
TGGAAGCTCAAGTGACACAGAACCCCAGATACCTGATCACCGTGACCGGCAAAA
AGCTGACCGTGACCTGTAGCCAGAACATGAACCACGAGTACATGAGCTGGTAT
CGGCAAGACCCTGGCCTGGGGCTGAGACAGATCTACTATAGCATGAACGTGGA
AGTGACCGACAAAGGCGACGTGCCCGAGGGCTATAAGGTGTCCCGGAAAGAGA
AGCGGAACTTTCCACTGATCCTGGAATCCCCATCTCCTAACCAGACCAGCCTGTA
TTTTTGCGCTAGTTCTGCCGGGACCGGGGGGCATGAGCAATACTTCGGGCCG
GGCACCAGGCTCACGGTCACAGaagatctgaacaaggtgttccctccagaggtggccgtgttcgagccttctaag
gccgagatcgcccacacacaaaaagccaccctcgtgtgcctggccaccggctttttccccgaccacgtggaactgtcttggtgggtca
acggcaaagaggtgcactccggcgtgtcaacggatccccagcctctgaaagaacagcctgccctgaacgacagccggtactgcctg
agctccagactgagagtgtccgccaccttctggcagaacccccggaaccacttcagatgccaggtgcagttttacggcctgagcgag
aacgacgagtggacccaggacagagccaagcccgtgacacaaatcgtgtctgccgaagcctggggaagagccgattgcggcatca
ccagcgcctcctatcaccagggcgtgctgagcgccacaatcctgtacgaaatcctgctgggcaaggccaccctgtacgccgtgctgg
tgtctgctctggtgctgatggccatggtcaagcggaaggactttggcagcggcagagccaaaaggtccgggagcggt
Beta chain protein sequence
MGPQLLGYVVLCLLGAGPLEAQVTQNPRYLITVTGKKLTVTCSQNMNHEYMSWYR
QDPGLGLRQIYYSMNVEVTDKGDVPEGYKVSRKEKRNFPLILESPSPNQTSLYFCAS
SAGTGGHEQYFGPGTRLTVTEdlnkvfppevavfepskaeiahtqkatlvclatgffpdhvelswwvngkevh
sgvstdpqplkeqpalndsryclssrlrvsatfwqnpmnhfreqvqfyglsendewtqdrakpvtqivsaeawgradcgitsasyh
qgvlsatilyeillgkatlyavlvsalvlmamvkrkdfgsgrakrsgsg
Complete Beta and Alpha ORF DNA Sequence (The underlined italic region in the “Furin-
P2A” site encodes a sequence allowing for expression of two polypeptide chains in a single
cassette)
ATGGGACCTCAGCTGCTGGGATATGTGGTGCTGTGTCTGCTCGGAGCTGGACCCC
TGGAAGCTCAAGTGACACAGAACCCCAGATACCTGATCACCGTGACCGGCAAAA
AGCTGACCGTGACCTGTAGCCAGAACATGAACCACGAGTACATGAGCTGGTAT
CGGCAAGACCCTGGCCTGGGGCTGAGACAGATCTACTATAGCATGAACGTGGA
AGTGACCGACAAAGGCGACGTGCCCGAGGGCTATAAGGTGTCCCGGAAAGAGA
AGCGGAACTTTCCACTGATCCTGGAATCCCCATCTCCTAACCAGACCAGCCTGTA
TTTTTGCGCTAGTTCTGCCGGGACCGGGGGGCATGAGCAATACTTCGGGCCG
GGCACCAGGCTCACGGTCACAGaagatctgaacaaggtgttccctccagaggtggccgtgttcgagccttctaag
gccgagatcgcccacacacaaaaagccaccctcgtgtgcctggccaccggctttttccccgaccacgtggaactgtcttggtgggtca
acggcaaagaggtgcactccggcgtgtcaacggatccccagcctctgaaagaacagcctgccctgaacgacagccggtactgcctg
agctccagactgagagtgtccgccaccttctggcagaacccccggaaccacttcagatgccaggtgcagttttacggcctgagcgag
aacgacgagtggacccaggacagagccaagcccgtgacacaaatcgtgtctgccgaagcctggggaagagccgattgcggcatca
ccagcgcctcctatcaccagggcgtgctgagcgccacaatcctgtacgaaatcctgctgggcaaggccaccctgtacgccgtgctgg
tgtctgctctggtgctgatggccatggtcaagcggaaggactttggcagcggcagagccaaaaggtccgggagcggtGCGAC
AAACTTTAGCCTGTTGAAACAAGCCGGCGACGTTGAAGAGAACCCCGGACCTATGGA
AACCCTGCTGAAGGTGCTGTCTGGCACCCTGCTGTGGCAGCTGACATGGGTCCGA
TCTCAGCAGCCTGTGCAGTCTCCTCAGGCCGTGATTCTGAGAGAAGGCGAGGAC
GCCGTGATCAACTGCAGCAGCTCTAAGGCCCTGTACAGCGTGCACTGGTACAG
GCAGAAACACGGCGAGGCCCCAGTGTTTCTGATGATTCTGCTGAAAGGCGGCG
AGCAGAAGGGCCACGATAAGATCTCCGCCAGCTTCAACGAGAAGAAGCAGCAG
TCCAGCCTGTACCTGACAGCCAGCCAGCTGAGCTACAGCGGCACCTATTTCTGTG
GCACAGAAGGTACTGGTGACTACAAGCTCTCTTTTGGAGCCGGAACCACAGT
AACTGTAAGAGCAAacatccagaaccccgaccccgccgtgtaccagctgagggactccaagtccagcgacaagagc
gtgtgtctgtttacggacttcgacagccagaccaacgtgagtcaaagcaaggacagcgacgtctacataacggataagaccgtgctg
gacatgcggagcatggacttcaagagcaacagcgccgtggcctggtccaacaagagcgacttcgcctgcgccaacgccttcaacaa
cagcatcatccccgaggacaccttcttccccagcagcgacgtgccctgcgacgtgaaactggtggagaagtccttcgagacagacac
caatctgaactttcagaacctgctggtgatcgtgctgcggattctgctgctgaaagtggccggcttcaatctgctgatgaccctgcggct
gtggagc
Complete Beta and Alpha ORF Protein Sequence (The underlined italic region in the “Furin-
P2A” site allows expression of two polypeptide chains in a single cassette)
MGPQLLGYVVLCLLGAGPLEAQVTQNPRYLITVTGKKLTVTCSQNMNHEYMSWYR
QDPGLGLRQIYYSMNVEVTDKGDVPEGYKVSRKEKRNFPLILESPSPNQTSLYFCAS
SAGTGGHEQYFGPGTRLTVTEdlnkvfppevavfepskaeiahtqkatlvclatgffpdhvelswwvngkevh
sgvstdpqplkeqpalndsryclssrlrvsatfwqnprnhfrcqvqfyglsendewtqdrakpvtqivsaeawgradcgitsasyh
qgvlsatilyeillgkatlyavlvsalvlmamvkrkdfgsgrakrsgsgATNFSLLKQAGDVEENPGPMETLLK
VLSGTLLWQLTWVRSQQPVQSPQAVILREGEDAVINCSSSKALYSVHWYRQKHGEA
PVFLMILLKGGEQKGHDKISASFNEKKQQSSLYLTASQLSYSGTYFCGTEGTGDYK
LSFGAGTTVTVRANiqnpdpavyqlrdskssdksvclftdfdsqtnvsqskdsdvyitdktvldmrsmdfksnsava
wsnksdfacanafnnsiipedtffpssdvpcdvklveksfetdtnlnfqnllvivirilllkvagfnllmtlrlws

* Table 2 provides, in part, representative TCR sequences are grouped according to MHC serotype presentation and sub-grouped according to different peptides presented by the MHC serotype and bound by the sub-grouped TCRs. Individual TCRs, such as those representatively exemplified in the tables, are described and claimed, as well as the genus of binding proteins that bind a peptide epitope sequence described herein either alone or in a complex with an MHC, such as those grouped in the tables provided herein. In addition, TRAV, TRAJ, and TRAC genes for each TCR alpha chain described herein, and TRBV, TRBJ, and TRBC genes for each TCR beta chain described herein, are provided. Sequences for each TCR described herein are provided as pairs of cognate alpha chain and beta chains for each named TCR. TCR sequences described herein are annotated. Variable domain sequences are capitalized. Constant domain sequences are in lower case. CDR1, CDR2, and CDR3 sequences are annotated using bold and underlined text. CDR1, CDR2, and CDR3 are shown in standard order of appearance from left (N-terminus) to right (C-terminus). TRAV, TRAJ, and TRAC genes for each TCR alpha chain described herein, and TRBV. TRBJ, and TRBC genes for each TCR beta chain described herein, are annotated according to well-known IMGT nomenclature described herein. Similarly, CDR1 and CDR2 of TRAV and TRBV are well-known in the art since they are based on well-known and annotated TRAV and TRBV sequences (e.g., as annotated in databases like IMGT available at imt.org and IEDB available at iedb.org).

```
TABLE 3
Human PRAME transcript variant 1 (NM_006115.5; CDS: 226-1755)
      1 acagctcccc cgcagccaga agccgggcct gcagcgcctc agcaccgctc cgggacaccc
    61 cacccgcttc ccaggcgtga cctgtcaaca gcaacttcgc ggtgtggtga actctctgag
  121 gaaaaaccat tttgattatt actctcagac gtgcgtggca acaagtgact gagacctaga
  181 aatccaagcg ttggaggtcc tgaggccagc ctaagtcgct tcaaaatgga acgaaggcgt
  241 ttgtggggtt ccattcagag ccgatacatc agcatgagtg tgtggacaag cccacggaga
  301 cttgtggagc tggcagggca gagcctgctg aaggatgagg ccctggccat tgccgccctg
  361 gagttgctgc ccagggagct cttcccgcca ctcttcatgg cagcctttga cgggagacac
  421 agccagaccc tgaaggcaat ggtgcaggcc tggcccttca cctgcctccc tctgggagtg
  481 ctgatgaagg gacaacatct tcacctggag accttcaaag ctgtgcttga tggacttgat
  541 gtgctccttg cccaggaggt tcgccccagg aggtggaaac ttcaagtgct ggatttacgg
  601 aagaactctc atcaggactt ctggactgta tggtctggaa acagggccag tctgtactca
  661 tttccagagc cagaagcagc tcagcccatg acaaagaagc gaaaagtaga tggtttgagc
  721 acagaggcag agcagccctt cattccagta gaggtgctcg tagacctgtt cctcaaggaa
  781 ggtgcctgtg atgaattgtt ctcctacctc attgagaaag tgaagcgaaa gaaaaatgta
  841 ctacgcctgt gctgtaagaa gctgaagatt tttgcaatgc ccatgcagga tatcaagatg
  901 atcctgaaaa tggtgcagct ggactctatt gaagatttgg aagtgacttg tacctggaag
  961 ctacccacct tggcgaaatt ttctccttac ctgggccaga tgattaatct gcgtagactc
1021 ctcctctccc acatccatgc atcttcctac atttccccgg agaaggaaga gcagtatatc
1081 gcccagttca cctctcagtt cctcagtctg cagtgcctgc aggctctcta tgtggactct
1141 ttatttttcc ttagaggccg cctggatcag ttgctcaggc acgtgatgaa ccccttggaa
1201 accctctcaa taactaactg ccggctttcg gaaggggatg tgatgcatct gtcccagagt
1261 cccagcgtca gtcagctaag tgtcctgagt ctaagtgggg tcatgctgac cgatgtaagt
1321 cccgagcccc tccaagctct gctggagaga gcctctgcca ccctccagga cctggtcttt
1381 gatgagtgtg ggatcacgga tgatcagctc cttgccctcc tgccttccct gagccactgc
1441 tcccagctta cgaccttaag cttctacggg aattccatct ccatatctgc cctgcagagt
1501 ctcctgcagc acctcatcgg gctgagcaat ctgacccacg tgctgtatcc tgtccccctg
1561 gagagttatg aggacatcca tggtaccctc cacctggaga ggcttgccta tctgcatgcc
1621 aggctcaggg agttcctgtg tgagttgggg cggcccagca tggtctggct tagtgccaac
1681 ccctgtcctc actgtgggga cagaaccttc tatgacccgg agcccatcct gtgcccctgt
1741 ttcatgccta attagctggg tgcacatatc aaatgcttca ttctgcatac ttggacacta
1801 aagccaggat gtgcatgcat cttgaagcaa caaagcagcc acagtttcag acaaatgttc
1861 agtgtgagtg aggaaaacat gttcagtgag gaaaaaacat tcagacaaat gttcagtgag
1921 gaaaaaaagg ggaagttggg ggtaggcaga tgttgacttg aggagttaat gtgatctttg
1981 gggagataca tcttatagag ttagaaatag aatctgaatt tctaaaggga gattctggct
2041 tgggaagtac atgtaggagt taatccctgt gtagactgtt gtaaagaaac tgttgaaaat
2101 aaagagaagc aatgtgaagc a
Human PRAME transcript variant 2 (NM_206953.3; CDS: 840-2369)
      1 acagctcccc cgcagccaga agccgggcct gcagcgcctc agcaccgctc cgggacaccc
    61 cacccgcttc ccaggcgtga cctgtcaaca gcaacttcgc ggtgtggtga actctctgag
  121 gaaaaacgta agttcgagcc ctgattcctc cgcttccccg cagggtgacc ttgggcttgt
  181 gcccccagca ccacccctgt cccgggtccc tgttttctct ctggaaatgg gttgaagacc
  241 aaagaaaata atgtgcgcca cttgggtcac cccgggccgc ctgccccgga aaattggccc
  301 cagttgagga gttgtggctg taaggatgcc ttgaaccgag gcggcggtgc tcgtggttgg
  361 agctctccag ggtgggtgcg catttgtaat gcggtggatg ctctgggact cggcccctct
  421 gaaggtgctg ggggttgggg acggcccagg cagtggcgta ggcgtcctag gaaggcggga
  481 gcagaggcag aaatgtcgct gcaagaccgt agtcagggtc cttgaccaca ggggtcactt
  541 gtgaccaacc acatggtctg ttgttcctcc tgccccctgg ttcagcccag gaaacactgg
  601 tgctcaggtt tggagccaga gatttgcact gaaagggcgg gattgagtcg ccagttgtca
  661 gtttcctcag cagtatttgc ggaggttttc acaggaggcc gttgcttcgt aaatattata
  721 catgtattct tctttttgga gcattttgat tattactctc agacgtgcgt ggcaacaagt
  781 gactgagacc tagaaatcca agcgttggag gtcctgaggc cagcctaagt cgcttcaaaa
  841 tggaacgaag gcgtttgtgg ggttccattc agagccgata catcagcatg agtgtgtgga
  901 caagcccacg gagacttgtg gagctggcag ggcagagcct gctgaaggat gaggccctgg
  961 ccattgccgc cctggagttg ctgcccaggg agctcttccc gccactcttc atggcagcct
1021 ttgacgggag acacagccag accctgaagg caatggtgca ggcctggccc ttcacctgcc
1081 tccctctggg agtgctgatg aagggacaac atcttcacct ggagaccttc aaagctgtgc
1141 ttgatggact tgatgtgctc cttgcccagg aggttcgccc caggaggtgg aaacttcaag
1201 tgctggattt acggaagaac tctcatcagg acttctggac tgtatggtct ggaaacaggg
1261 ccagtctgta ctcatttcca gagccagaag cagctcagcc catgacaaag aagcgaaaag
1321 tagatggttt gagcacagag gcagagcagc ccttcattcc agtagaggtg ctcgtagacc
1381 tgttcctcaa ggaaggtgcc tgtgatgaat tgttctccta cctcattgag aaagtgaagc
1441 gaaagaaaaa tgtactacgc ctgtgctgta agaagctgaa gatttttgca atgcccatgc
1501 aggatatcaa gatgatcctg aaaatggtgc agctggactc tattgaagat ttggaagtga
1561 cttgtacctg gaagctaccc accttggcga aattttctcc ttacctgggc cagatgatta
1621 atctgcgtag actcctcctc tcccacatcc atgcatcttc ctacatttcc ccggagaagg
1681 aagagcagta tatcgcccag ttcacctctc agttcctcag tctgcagtgc ctgcaggctc
1741 tctatgtgga ctctttattt ttccttagag gccgcctgga tcagttgctc aggcacgtga
1801 tgaacccctt ggaaaccctc tcaataacta actgccggct ttcggaaggg gatgtgatgc
1861 atctgtccca gagtcccagc gtcagtcagc taagtgtcct gagtctaagt ggggtcatgc
1921 tgaccgatgt aagtcccgag cccctccaag ctctgctgga gagagcctct gccaccctcc
1981 aggacctggt ctttgatgag tgtgggatca cggatgatca gctccttgcc ctcctgcctt
2041 ccctgagcca ctgctcccag cttacgacct taagcttcta cgggaattcc atctccatat
2101 ctgccctgca gagtctcctg cagcacctca tcgggctgag caatctgacc cacgtgctgt
2161 atcctgtccc cctggagagt tatgaggaca tccatggtac cctccacctg gagaggcttg
2221 cctatctgca tgccaggctc agggagttgc tgtgtgagtt ggggcggccc agcatggtct
2281 ggcttagtgc caacccctgt cctcactgtg gggacagaac cttctatgac ccggagccca
2341 tcctgtgccc ctgtttcatg cctaattagc tgggtgcaca tatcaaatgc ttcattctgc
2401 atacttggac actaaagcca ggatgtgcat gcatcttgaa gcaacaaagc agccacagtt
2461 tcagacaaat gttcagtgtg agtgaggaaa acatgttcag tgaggaaaaa acattcagac
2521 aaatgttcag tgaggaaaaa aaggggaagt tgggggtagg cagatgttga cttgaggagt
2581 taatgtgatc tttggggaga tacatcttat agagttagaa atagaatctg aatttctaaa
2641 gggagattct ggcttgggaa gtacatgtag gagttaatcc ctgtgtagac tgttgtaaag
2701 aaactgttga aaataaagag aagcaatgtg aagca
Human PRAME transcript variant 3 (NM_206954.3; CDS: 205-1734)
      1 acagctcccc cgcagccaga agccgggcct gcagcgcctc agcaccgctc cgggacaccc
    61 cacccgcttc ccaggcgtga cctgtcaaca gcaacttcgc ggtgtggtga actctctgag
  121 gaaaaacacg tgcgtggcaa caagtgactg agacctagaa atccaagcgt tggaggtcct
  181 gaggccagcc taagtcgctt caaaatggaa cgaaggcgtt tgtggggttc cattcagagc
  241 cgatacatca gcatgagtgt gtggacaagc ccacggagac ttgtggagct ggcagggcag
  301 agcctgctga aggatgaggc cctggccatt gccgccctgg agttgctgcc cagggagctc
  361 ttcccgccac tcttcatggc agcctttgac gggagacaca gccagaccct gaaggcaatg
  421 gtgcaggcct ggcccttcac ctgcctccct ctgggagtgc tgatgaaggg acaacatctt
  481 cacctggaga ccttcaaagc tgtgcttgat ggacttgatg tgctccttgc ccaggaggtt
  541 cgccccagga ggtggaaact tcaagtgctg gatttacgga agaactctca tcaggacttc
  601 tggactgtat ggtctggaaa cagggccagt ctgtactcat ttccagagcc agaagcagct
  661 cagcccatga casagaagcg aaaagtagat ggtttgagca cagaggcaga gcagcccttc
  721 attccagtag aggtgctcgt agacctgttc ctcaaggaag gtgcctgtga tgaattgttc
  781 tcctacctca ttgagaaagt gaagcgaaag aaaaatgtac tacgcctgtg ctgtaagaag
  841 ctgaagattt ttgcaatgcc catgcaggat atcaagatga tcctgaaaat ggtgcagctg
  901 gactctattg aagatttgga agtgacttgt acctggaagc tacccacctt ggcgaaattt
  961 tctccttacc tgggccagat gattaatctg cgtagactcc tcctctccca catccatgca
1021 tcttcctaca tttccccgga gaaggaagag cagtatatcg cccagttcac ctctcagttc
1081 ctcagtctgc agtgcctgca ggctctctat gtggactctt tatttttcct tagaggccgc
1141 ctggatcagt tgctcaggca cgtgatgaac cccttggaaa ccctctcaat aactaactgc
1201 cggctttcgg aaggggatgt gatgcatctg tcccagagtc ccagcgtcag tcagctaagt
1261 gtcctgagtc taagtggggt catgctgacc gatgtaagtc ccgagcccct ccaagctctg
1321 ctggagagag cctctgccac cctccaggac ctggtctttg atgagtgtgg gatcacggat
1381 gatcagctcc ttgccctcct gccttccctg agccactgct cccagcttac gaccttaagc
1441 ttctacggga attccatctc catatctgcc ctgcagagtc tcctgcagca cctcatcggg
1501 ctgagcaatc tgacccacgt gctgtatcct gtccccctgg agagttatga ggacatccat
1561 ggtaccctcc acctggagag gcttgcctat ctgcatgcca ggctcaggga gttgctgtgt
1621 gagttggggc ggcccagcat ggtctggctt agtgccaacc cctgtcctca ctgtggggac
1681 agaaccttct atgacccgga gcccatcctg tgcccctgtt tcatgcctaa ttagctgggt
1741 gcacatatca aatgcttcat tctgcatact tggacactaa agccaggatg tgcatgcatc
1801 ttgaagcaac aaagcagcca cagtttcaga caaatgttca gtgtgagtga ggaaaacatg
1861 ttcagtgagg aaaaaacatt cagacaaatg ttcagtgagg aaaaaaaggg gaagttgggg
1921 gtaggcagat gttgacttga ggagttaatg tgatctttgg ggagatacat cttatagagt
1981 tagaaataga atctgaattt ctaaagggag attctggctt gggaagtaca tgtaggagtt
2041 aatccctgtg tagactgttg taaagaaact gttgaaaata aagagaagca atgtgaagca
Human PRAME transcript variant 4 (NM_206955.3; CDS: 430-1959)
      1 acagctcccc cgcagccaga agccgggcct gcagcgcctc agcaccgctc cgggacaccc
    61 cacccgcttc ccaggcgtga cctgtcaaca ggtctgtatt ggcgacaaaa ggagcagccc
  121 tgaatgtagg gaaagcaggg cggagtcctc tgcaggctcg ggggagggga ggggcgtgaa
  181 tgcgtggatt tctgtggaga gtggaaacac ggggagtcga ggggagcatg cgcgggcctc
  241 agaaagttct gggaaaccga ctcccgggag cagggaggaa cgcgcgctcc agagacaact
  301 tcgcggtgtg gtgaactctc tgaggaaaaa ccattttgat tattactctc agacgtgcgt
  361 ggcaacaagt gactgagacc tagaaatcca agcgttggag gtcctgaggc cagcctaagt
  421 cgcttcaaaa tggaacgaag gcgtttgtgg ggttccattc agagccgata catcagcatg
  481 agtgtgtgga caagcccacg gagacttgtg gagctggcag ggcagagcct gctgaaggat
  541 gaggccctgg ccattgccgc cctggagttg ctgcccaggg agctcttccc gccactcttc
  601 atggcagcct ttgacgggag acacagccag accctgaagg caatggtgca ggcctggccc
  661 ttcacctgcc tccctctggg agtgctgatg aagggacaac atcttcacct ggagaccttc
  721 aaagctgtgc ttgatggact tgatgtgctc cttgcccagg aggttcgccc caggaggtgg
  781 aaacttcaag tgctggattt acggaagaac tctcatcagg acttctggac tgtatggtct
  841 ggaaacaggg ccagtctgta ctcatttcca gagccagaag cagctcagcc catgacaaag
  901 aagcgaaaag tagatggttt gagcacagag gcagagcagc ccttcattcc agtagaggtg
  961 ctcgtagacc tgttcctcaa ggaaggtgcc tgtgatgaat tgttctccta cctcattgag
1021 aaagtgaagc gaaagaaaaa tgtactacgc ctgtgctgta agaagctgaa gatttttgca
1081 atgcccatgc aggatatcaa gatgatcctg aaaatggtgc agctggactc tattgaagat
1141 ttggaagtga cttgtacctg gaagctaccc accttggcga aattttctcc ttacctgggc
1201 cagatgatta atctgcgtag actcctcctc tcccacatcc atgcatcttc ctacatttcc
1261 ccggagaagg aagagcagta tatcgcccag ttcacctctc agttcctcag tctgcagtgc
1321 ctgcaggctc tctatgtgga ctctttattt ttccttagag gccgcctgga tcagttgctc
1381 aggcacgtga tgaacccctt ggaaaccctc tcaataacta actgccggct ttcggaaggg
1441 gatgtgatgc atctgtccca gagtcccagc gtcagtcagc taagtgtcct gagtctaagt
1501 ggggtcatgc tgaccgatgt aagtcccgag cccctccaag ctctgctgga gagagcctct
1561 gccaccctcc aggacctggt ctttgatgag tgtgggatca cggatgatca gctccttgcc
1621 ctcctgcctt ccctgagcca ctgctcccag cttacgacct taagcttcta cgggaattcc
1681 atctccatat ctgccctgca gagtctcctg cagcacctca tcgggctgag caatctgacc
1741 cacgtgctgt atcctgtccc cctggagagt tatgaggaca tccatggtac cctccacctg
1801 gagaggcttg cctatctgca tgccaggctc agggagttgc tgtgtgagtt ggggcggccc
1861 agcatggtct ggcttagtgc caacccctgt cctcactgtg gggacagaac cttctatgac
1921 ccggagccca tcctgtgccc ctgtttcatg cctaattagc tgggtgcaca tatcaaatgc
1981 ttcattctgc atacttggac actaaagcca ggatgtgcat gcatcttgaa gcaacaaagc
2041 agccacagtt tcagacaaat gttcagtgtg agtgaggaaa acatgttcag tgaggaaaaa
2101 acattcagac aaatgttcag tgaggaaaaa aaggggaagt tgggggtagg cagatgttga
2161 cttgaggagt taatgtgatc tttggggaga tacatcttat agagttagaa atagaatctg
2221 aatttctaaa gggagattct ggcttgggaa gtacatgtag gagttaatcc ctgtgtagac
2281 tgttgtaaag aaactgttga aaataaagag aagcaatgtg aagca
Human PRAME transcript variant 5 (NM_206956.3; 409-1938)
      1 acagctcccc cgcagccaga agccgggcct gcagcgcctc agcaccgctc cgggacaccc
    61 cacccgcttc ccaggcgtga cctgtcaaca ggtctgtatt ggcgacaaaa ggagcagccc
  121 tgaatgtagg gaaagcaggg cggagtcctc tgcaggctcg ggggagggga ggggcgtgaa
  181 tgcgtggatt tctgtggaga gtggaaacac ggggagtcga ggggagcatg cgcgggcctc
  241 agaaagttct gggaaaccga ctcccgggag cagggaggaa cgcgcgctcc agagacaact
  301 tcgcggtgtg gtgaactctc tgaggaaaaa cacgtgcgtg gcaacaagtg actgagacct
  361 agaaatccaa gcgttggagg tcctgaggcc agcctaagtc gcttcaaaat ggaacgaagg
  421 cgtttgtggg gttccattca gagccgatac atcagcatga gtgtgtggac aagcccacgg
  481 agacttgtgg agctggcagg gcagagcctg ctgaaggatg aggccctggc cattgccgcc
  541 ctggagttgc tgcccaggga gctcttcccg ccactcttca tggcagcctt tgacgggaga
  601 cacagccaga ccctgaaggc aatggtgcag gcctggccct tcacctgcct ccctctggga
  661 gtgctgatga agggacaaca tcttcacctg gagaccttca aagctgtgct tgatggactt
  721 gatgtgctcc ttgcccagga ggttcgcccc aggaggtgga aacttcaagt gctggattta
  781 cggaagaact ctcatcagga cttctggact gtatggtctg gaaacagggc cagtctgtac
  841 tcatttccag agccagaagc agctcagccc atgacaaaga agcgaaaagt agatggtttg
  901 agcacagagg cagagcagcc cttcattcca gtagaggtgc tcgtagacct gttcctcaag
  961 gaaggtgcct gtgatgaatt gttctcctac ctcattgaga aagtgaagcg aaagaaaaat
1021 gtactacgcc tgtgctgtaa gaagctgaag atttttgcaa tgcccatgca ggatatcaag
1081 atgatcctga aaatggtgca gctggactct attgaagatt tggaagtgac ttgtacctgg
1141 aagctaccca ccttggcgaa attttctcct tacctgggcc agatgattaa tctgcgtaga
1201 ctcctcctct cccacatcca tgcatcttcc tacatttccc cggagaagga agagcagtat
1261 atcgcccagt tcacctctca gttcctcagt ctgcagtgcc tgcaggctct ctatgtggac
1321 tctttatttt tccttagagg ccgcctggat cagttgctca ggcacgtgat gaaccccttg
1381 gaaaccctct caataactaa ctgccggctt tcggaagggg atgtgatgca tctgtcccag
1441 agtcccagcg tcagtcagct aagtgtcctg agtctaagtg gggtcatgct gaccgatgta
1501 agtcccgagc ccctccaagc tctgctggag agagcctctg ccaccctcca ggacctggtc
1561 tttgatgagt gtgggatcac ggatgatcag ctccttgccc tcctgccttc cctgagccac
1621 tgctcccagc ttacgacctt aagcttctac gggaattcca tctccatatc tgccctgcag
1681 agtctcctgc agcacctcat cgggctgagc aatctgaccc acgtgctgta tcctgtcccc
1741 ctggagagtt atgaggacat ccatggtacc ctccacctgg agaggcttgc ctatctgcat
1801 gccaggctca gggagttgct gtgtgagttg gggcggccca gcatggtctg gcttagtgcc
1861 aacccctgtc ctcactgtgg ggacagaacc ttctatgacc cggagcccat cctgtgcccc
1921 tgtttcatgc ctaattagct gggtgcacat atcaaatgct tcattctgca tacttggaca
1981 ctaaagccag gatgtgcatg catcttgaag caacaaagca gccacagttt cagacaaatg
2041 ttcagtgtga gtgaggaaaa catgttcagt gaggaaaaaa cattcagaca aatgttcagt
2101 gaggaaaaaa aggggaagtt gggggtaggc agatgttgac ttgaggagtt aatgtgatct
2161 ttggggagat acatcttata gagttagaaa tagaatctga atttctaaag ggagattctg
2221 gcttgggaag tacatgtagg agttaatccc tgtgtagact gttgtaaaga aactgttgaa
2281 aataaagaga agcaatgtga agca
Human PRAME transcript variant 6 (NM_001291715.2; CDS: 187-1716)
      1 acgcagcggc tagggcgctt agctgagcca ttgtctcgtt ctttccttcc agcaacttcg
    61 cggtgtggtg aactctctga ggaaaaacca ttttgattat tactctcaga cgtgcgtggc
  121 aacaagtgac tgagacctag aaatccaagc gttggaggtc ctgaggccag cctaagtcgc
  181 ttcaaaatgg aacgaaggcg tttgtggggt tccattcaga gccgatacat cagcatgagt
  241 gtgtggacaa gcccacggag acttgtggag ctggcagggc agagcctgct gaaggatgag
  301 gccctggcca ttgccgccct ggagttgctg cccagggagc tcttcccgcc actcttcatg
  361 gcagcctttg acgggagaca cagccagacc ctgaaggcaa tggtgcaggc ctggcccttc
  421 acctgcctcc ctctgggagt gctgatgaag ggacaacatc ttcacctgga gaccttcaaa
  481 gctgtgcttg atggacttga tgtgctcctt gcccaggagg ttcgccccag gaggtggaaa
  541 cttcaagtgc tggatttacg gaagaactct catcaggact tctggactgt atggtctgga
  601 aacagggcca gtctgtactc atttccagag ccagaagcag ctcagcccat gacaaagaag
  661 cgaaaagtag atggtttgag cacagaggca gagcagccct tcattccagt agaggtgctc
  721 gtagacctgt tcctcaagga aggtgcctgt gatgaattgt tctcctacct cattgagaaa
  781 gtgaagcgaa agaaaaatgt actacgcctg tgctgtaaga agctgaagat ttttgcaatg
  841 cccatgcagg atatcaagat gatcctgaaa atggtgcagc tggactctat tgaagatttg
  901 gaagtgactt gtacctggaa gctacccacc ttggcgaaat tttctcctta cctgggccag
  961 atgattaatc tccgtagact cctcctctcc cacatccatg catcttccta catttccccg
1021 gagaaggaag agcagtatat cgcccagttc acctctcagt tcctcagtct gcagtgcctg
1081 caggctctct atgtggactc tttatttttc cttagaggcc gcctggatca gttgctcagg
1141 cacgtgatga accccttgga aaccctctca ataactaact gccggctttc ggaaggggat
1201 gtgatgcatc tgtcccagag tcccagcgtc agtcagctaa gtgtcctgag tctaagtggg
1261 gtcatgctga ccgatgtaag tcccgagccc ctccaagctc tgctggagag agcctctgcc
1321 accctccagg acctggtctt tgatgagtgt gggatcacgg atgatcagct ccttgccctc
1381 ctgccttccc tgagccactg ctcccagctt acgaccttaa gcttctacgg gaattccatc
1441 tccatatctg ccctgcagag tctcctgcag cacctcatcg ggctgagcaa tctgacccac
1501 gtgctgtatc ctgtccccct ggagagttat gaggacatcc atggtaccct ccacctggag
1561 aggcttgcct atctgcatgc caggctcagg gagttgctgt gtgagttggg gcggcccagc
1621 atggtctggc ttagtgccaa cccctgtcct cactgtgggg acagaacctt ctatgacccg
1681 gagcccatcc tgtgcccctg tttcatgcct aattagctgg gtgcacatat caaatgcttc
1741 attctgcata cttggacact aaagccagga tgtgcatgca tcttgaagca acaaagcagc
1801 cacagtttca gacaaatgtt cagtgtgagt gaggaaaaca tgttcagtga ggaaaaaaca
1861 ttcagacasa tgttcagtga ggaaaaaaag gggaagttgg gggtaggcag atgttgactt
1921 gaggagttaa tgtgatcttt ggggagatac atcttataga gttagaaata gaatctgaat
1981 ttctaaaggg agattctggc ttgggaagta catgtaggag ttaatccctg tgtagactgt
2041 tgtaaagaaa ctgttgaaaa taaagagaag caatgtgaag ca
Human PRAME transcript variant 7 (NM_001291716.2; 166-1695)
      1 acgcagcggc tagggcgctt agctgagcca ttgtctcgtt ctttccttcc agcaacttcg
    61 cggtgtggtg aactctctga ggaaaaacac gtgcgtggca acaagtgact gagacctaga
  121 aatccaagcg ttggaggtcc tgaggccagc ctaagtcgct tcaaaatgga acgaaggcgt
  181 ttgtggggtt ccattcagag ccgatacatc agcatgagtg tgtggacaag cccacggaga
  241 cttgtggagc tggcagggca gagcctgctg aaggatgagg ccctggccat tgccgccctg
  301 gagttgctgc ccagggagct cttcccgcca ctcttcatgg cagcctttga cgggagacac
  361 agccagaccc tgaaggcaat ggtgcaggcc tggcccttca cctgcctccc tctgggagtg
  421 ctgatgaagg gacaacatct tcacctggag accttcaaag ctgtgcttga tggacttgat
  481 gtgctccttg cccaggaggt tcgccccagg aggtggaaac ttcaagtgct ggatttacgg
  541 aagaactctc atcaggactt ctggactgta tggtctggaa acagggccag tctgtactca
  601 tttccagagc cagaagcagc tcagcccatg acaaagaagc gaaaagtaga tggtttgagc
  661 acagaggcag agcagccctt cattccagta gaggtgctcg tagacctgtt cctcaaggaa
  721 ggtgcctgtg atgaattgtt ctcctacctc attgagaaag tgaagcgaaa gaaaaatgta
  781 ctacgcctgt gctgtaagaa gctgaagatt tttgcaatgc ccatgcagga tatcaagatg
  841 atcctgaaaa tcgtgcagct ggactctatt gaagatttgg aagtgacttg tacctggaag
  901 ctacccacct tggcgaaatt ttctccttac ctgggccaga tgattaatct gcgtagactc
  961 ctcctctccc acatccatgc atcttcctac atttccccgg agaaggaaga gcagtatatc
1021 gcccagttca cctctcagtt cctcagtctg cagtgcctgc aggctctcta tgtggactct
1081 ttatttttcc ttagaggccg cctggatcag ttgctcaggc acgtgatgaa ccccttggaa
1141 accctctcaa taactaactg ccggctttcg gaaggggatg tgatgcatct gtcccagagt
1201 cccagcgtca gtcagctaag tgtcctgagt ctaagtgggg tcatgctgac cgatgtaagt
1261 cccgagcccc tccaagctct gctggagaga gcctctgcca ccctccagga cctggtcttt
1321 gatgagtgtg ggatcacgga tgatcagctc cttgccctcc tgccttccct gagccactgc
1381 tcccagctta cgaccttaag cttctacggg aattccatct ccatatctgc cctgcagagt
1441 ctcctgcagc acctcatcgg gctgagcaat ctgacccacg tgctgtatcc tgtccccctg
1501 gagagttatg aggacatcca tggtaccctc cacctggaga ggcttgccta tctgcatgcc
1561 aggctcaggg agttgctgtg tgagttgggg cggcccagca tggtctggct tagtgccaac
1621 ccctgtcctc actgtgggga cagaaccttc tatgacccgg agcccatcct gtgcccctgt
1681 ttcatgccta attagctggg tgcacatatc aaatgcttca ttctgcatac ttggacacta
1741 aagccaggat gtgcatgcat cttgaagcaa caaagcagcc acagtttcag acaaatgttc
1801 agtgtgagtg aggaaaacat gttcagtgag gaaaaaacat tcagacaaat gttcagtgag
1861 gaaaaaaagg ggaagttggg ggtaggcaga tgttgacttg aggagttaat gtgatctttg
1921 gggagataca tcttatagag ttagaaatag aatctgaatt tctaaaggga gattctggct
1981 tgggaagtac atgtaggagt taatccctgt gtagactgtt gtaaagaaac tttgasaat
2041 aaagagaagc aatgtgaagc a
Human PRAME transcript variant 8 (NM_001291717.2; CDS: 359 . . . 1840)
      1 acagctcccc cgcagccaga agccgggcct gcagcgcctc agcaccgctc cgggacaccc
    61 cacccgcttc ccaggcgtga cctgtcaaca ggtctgtatt ggcgacaaaa ggagcagccc
  121 tgaatgtagg gaaagcaggg cggagtcctc tgcaggctcg ggggagggga ggggcgtgaa
  181 tgcgtggatt tctgtggaga gtggaaacac ggggagtcga ggggagcatg cgcgggcctc
  241 agaaagttct gggaaaccga ctcccgggag cagggaggaa cgcgcgctcc agagacaact
  301 tcgcggtgtg gtgaactctc tgaggaaaaa cggttccatt cagagccgat acatcagcat
  361 gagtgtgtgg acaagcccac ggagacttgt ggagctggca gggcagagcc tgctgaagga
  421 tgaggccctg gccattgccg ccctggagtt gctgcccagg gagctcttcc cgccactctt
  481 catggcagcc tttgacggga gacacagcca gaccctgaag gcaatggtgc aggcctggcc
  541 cttcacctgc ctccctctgg gagtgctgat gaagggacaa catcttcacc tggagacctt
  601 caaagctgtg cttgatggac ttgatgtgct ccttgcccag gaggttcgcc ccaggaggtg
  661 gaaacttcaa gtgctggatt tacggaagaa ctctcatcag gacttctgga ctgtatggtc
  721 tggaaacagg gccagtctgt actcatttcc agagccagaa gcagctcagc ccatgacaaa
  781 gaagcgaaaa gtagatggtt tgagcacaga ggcagagcag cccttcattc cagtagaggt
  841 gctcgtagac ctgttcctca aggaaggtgc ctgtgatgaa ttgttctcct acctcattga
  901 gaaagtgaag cgaaagaaaa atgtactacg cctgtgctgt aagaagctga agatttttgc
  961 aatgcccatg caggatatca agatgatcct gaaaatggtg cagctggact ctattgaaga
1021 tttggaagtg acttgtacct ggaagctacc caccttggcg aaattttctc cttacctggg
1081 ccagatgatt aatctgcgta gactcctcct ctcccacatc catgcatctt cctacatttc
1141 cccggagaag gaagagcagt atatcgccca gttcacctct cagttcctca gtctgcagtg
1201 cctgcaggct ctctatgtgg actctttatt tttccttaga ggccgcctgg atcagttgct
1261 caggcacgtg atgaacccct tggaaaccct ctcaataact aactgccggc tttcggaagg
1321 ggatgtgatg catctgtccc agagtcccag cgtcagtcag ctaagtgtcc tgagtctaag
1381 tggggtcatg ctgaccgatg taagtcccga gcccctccaa gctctgctgg agagagcctc
1441 tgccaccctc caggacctgg tctttgatga gtgtgggatc acggatgatc agctccttgc
1501 cctcctgcct tccctgagcc actgctccca gcttacgacc ttaagcttct acgggaattc
1561 catctccata tctgccctgc agagtctcct gcagcacctc atcgggctga gcaatctgac
1621 ccacgtgctg tatcctgtcc ccctggagag ttatgaggac atccatggta ccctccacct
1681 ggagaggctt gcctatctgc atgccaggct cagggagttg ctgtgtgagt tggggcggcc
1741 cagcatggtc tggcttagtg ccaacccctg tcctcactgt ggggacagaa ccttctatga
1801 cccggagccc atcctgtgcc cctgtttcat gcctaattag ctgggtgcac atatcaaatg
1861 cttcattctg catacttgga cactaaagcc aggatgtgca tgcatcttga agcaacaaag
1921 cagccacagt ttcagacaaa tgttcagtgt gagtgaggaa aacatgttca gtgaggaaaa
1981 aacattcaga caaatgttca gtgaggaaaa aaaggggaag ttgggggtag gcagatgttg
2041 acttgaggag ttaatgtgat ctttggggag atacatctta tagagttaga aatagaatct
2101 gaatttctaa agggagattc tggcttggga agtacatgta ggagttaatc cctgtgtaga
2161 ctgttgtaaa gaaactgttg aaaataaaga gaagcaatgt gaagca
Human PRAME transcript variant 9 (NM_001291719.2; CDS: 116-1597)
      1 acgcagcggc tagggcgctt agctgagcca ttgtctcgtt ctttccttcc agcaacttcg
    61 cggtgtggtg aactctctga ggaaaaacgg ttccattcag agccgataca tcagcatgag
  121 tgtgtggaca agcccacgga gacttgtgga gctggcaggg cagagcctgc tgaaggatga
  181 ggccctggcc attgccgccc tggagttgct gcccagggag ctcttcccgc cactcttcat
  241 ggcagccttt gacgggagac acagccagac cctgaaggca atggtgcagg cctggccctt
  301 cacctgcctc cctctgggag tgctgatgaa gggacaacat cttcacctgg agaccttcaa
  361 agctgtgctt gatggacttg atgtgctcct tgcccaggag gttcgcccca ggaggtggaa
  421 acttcaagtg ctggatttac ggaagaactc tcatcaggac ttctggactg tatggtctgg
  481 aaacagggcc agtctgtact catttccaga gccagaagca gctcagccca tgacaaagaa
  541 gcgaaaagta gatggtttga gcacagaggc agagcagccc ttcattccag tagaggtgct
  601 cgtagacctg ttcctcaagg aaggtgcctg tgatgaattg ttctcctacc tcattgagaa
  661 agtgaagcga aagaaaaatg tactacgcct gtgctgtaag aagctgaaga tttttgcaat
  721 gcccatgcag gatatcaaga tgatcctgaa aatggtgcag ctggactcta ttgaagattt
  781 ggaagtgact tgtacctgga agctacccac cttggcgaaa ttttctcctt acctgggcca
  841 gatgattaat ctgcgtagac tcctcctctc ccacatccat gcatcttcct acatttcccc
  901 ggagaaggaa gagcagtata tcgcccagtt cacctctcag ttcctcagtc tgcagtgcct
  961 gcaggctctc tatgtggact ctttattttt ccttagaggc cgcctggatc agttgctcag
1021 gcacgtgatg aaccccttgg aaaccctctc aataactaac tgccggcttt cggaagggga
1081 tgtgatgcat ctgtcccaga gtcccagcgt cagtcagcta agtgtcctga gtctaagtgg
1141 ggtcatgctg accgatgtaa gtcccgagcc cctccaagct ctgctggaga gagcctctgc
1201 caccctccag gacctggtct ttgatgagtg tgggatcacg gatgatcagc tccttgccct
126: cctgccttcc ctgagccact gctcccagct tacgacctta agcttctacg ggaattccat
1321 ctccatatct gccctgcaga gtctcctgca gcacctcatc gggctgagca atctgaccca
1381 cgtgctgtat cctgtccccc tggagagtta tgaggacatc catggtaccc tccacctgga
1441 gaggcttgcc tatctgcatg ccaggctcag ggagttgctg tgtgagttgg ggcggcccag
1501 catggtctgg cttagtgcca acccctgtcc tcactgtggg gacagaacct tctatgaccc
1561 ggagcccatc ctgtgcccct gtttcatgcc taattagctg ggtgcacata tcaaatgctt
1621 cattctgcat acttggacac taaagccagg atgtgcatgc atcttgaagc aacaaagcag
1681 ccacagtttc agacaaatgt tcagtgtgag tgaggaaaac atgttcagtg aggaaaaaac
1741 attcagacaa atgttcagtg aggaaaaaaa ggggaagttg ggggtaggca gatgttgact
1801 tgaggagtta atgtgatctt tggggagata catcttatag agttagaaat agaatctgaa
1861 tttctaaagg gagattctgg cttgggaagt acatgtagga gttaatccct gtgtagactg
1921 ttgtaaagaa actgttgaaa ataaagagaa gcaatgtgaa gca
Human PRAME transcript variant 10 (NM_001318126.2; CDS: 92-414)
      1 agacgcaaaa gcccacactt ccagtggtgt cagagagtat gagctccagg aggctttgat
    61 ctcactgccc catgggctty ggccattagg gggttccatt cagagccgat acatcagcat
  121 gagtgtgtgg acaagcccac ggagacttgt ggagctggca gggcagagcc tgctgaagga
  181 tgaggccctg gccattgccg ccctggagtt gctgcccagg gagctcttcc cgccactctt
  241 catggcagcc tttgacggga gacacagcca gaccctgaag gcaatggtgc aggcctggcc
  301 cttcacctgc ctccctctgg gagtgctgat gaagggacaa catcttcacc tggagacctt
  361 caaagctgtg cttgatggac ttgatgtgct ccttgcccag gaggttcgcc ccaggaggtg
  421 gaaacttcaa gtgctggatt tacggaagaa ctctcatcag gacttctgga ctgtatggtc
  481 tggaaacagg gccagtctgt actcatttcc agagccagaa gcagctcagc ccatgacaaa
  541 gaagcgaaaa gtagatggtt tgagcacaga ggcagagcag cccttcattc cagtagaggt
  601 gctcgtagac ctgttcctca aggaaggtgc ctgtgatgaa ttgttctcct acctcattga
  661 gaaagtgaag cgaaagaaaa atgtactacg cctgtgctgt aagaagctga agatttttgc
  721 aatgcccatg caggatatca agatgatcct gaaaatggtg cagctggact ctattgaaga
  781 tttggaagtg acttgtacct ggaagctacc caccttggcg aaattttctc cttacctggg
  841 ccagatgatt aatctgcgta gactcctcct ctcccacatc catgcatctt cctacatttc
  901 cccggagaag gaagagcagt atatcgccca gttcacctct cagttcctca gtctgcagtg
  961 cctgcaggct ctctatgtgg actctttatt tttccttaga ggccgcctgg atcagttgct
1021 caggcacgtg atgaacccct tggaaaccct ctcaataact aactgccggc tttcggaagg
1081 ggatgtgatg catctgtccc agagtcccag cgtcagtcag ctaagtgtcc tgagtctaag
1141 tggggtcatg ctgaccgatg taagtcccga gcccctccaa gctctgctgg agagagcctc
1201 tgccaccctc caggacctgg tctttgatga gtgtgggatc acggatgatc agctccttgc
1261 cctcctgcct tccctgagcc actgctccca gcttacgacc ttaagcttct acgggaattc
1321 catctccata tctgccctgc agagtctcct gcagcacctc atcgggctga gcaatctgac
1381 ccacgtgctg tatcctgtcc ccctggagag ttatgaggac atccatggta ccctccacct
1441 ggagaggctt gcctatctgc atgccaggct cagggagttg ctgtgtgagt tggggcggcc
1501 cagcatggtc tggcttagtg ccaacccctg tcctcactgt ggggacagaa ccttctatga
1561 cccggagccc atcctgtgcc cctgtttcat gcctaattag ctgggtgcac atatcaaatg
1621 cttcattctg catacttgga cactaaagcc aggatgtgca tgcatcttga agcaacaaag
1681 cagccacagt ttcagacaaa tgttcagtgt gagtgaggaa aacatgttca gtgaggaaaa
1741 aacattcaga caaatgttca gtgaggaaaa aaaggggaag ttgggggtag gcagatgttg
1801 acttgaggag ttaatgtgat ctttggggag atacatctta tagagttaga aatagaatct
1861 gaatttctaa agggagattc tggcttggga agtacatgta ggagttaatc cctgtgtaga
1921 ctgttgtaaa gaaactgttg aaaataaaga gaagcaatgt gaagca
Human PRAME transcript variant 11 (NM_001318127.2; CDS: 381-1862)
      1 aaaagcacca gtgggtgatc aggcccagga taggaatcat ccctaagcag atccttaaat
    61 gccaaatata acttcaattt tttgtgacgg aaaatgtatt tgtgcactgt gcctagcatg
  121 gaaatagaga aaaactgagt ctaattgata ggcctgattg catctcgctg accagaatct
  181 ctgtacctga gcagcttagg ggcaagaaag aacagctgtt ttcttgaagt gtggaaacat
  241 tagtaatact taggaagggg ctggccattg gagattatgt ttgtggtaaa ggcctgacct
  301 gctgcttgtg aacagcccca gtgtggaaga ggacacctaa ccataaagag aagggttcca
  361 ttcagagccg atacatcagc atgagtgtgt ggacaagccc acggagactt gtggagctgg
  421 cagggcagag cctgctgaag gatgaggccc tggccattgc cgccctggag ttgctgccca
  481 gggagctctt cccgccactc ttcatggcag cctttgacgg gagacacagc cagaccctga
  541 aggcaatggt gcaggcctgg cccttcacct gcctccctct gggagtgctg atgaagggac
  601 aacatcttca cctggagacc ttcaaagctg tgcttgatgg acttgatgtg ctccttgccc
  661 aggaggttcg ccccaggagg tggaaacttc aagtgctgga tttacggaag aactctcatc
  721 aggacttctg gactgtatgg tctggaaaca gggccagtct gtactcattt ccagagccag
  781 aagcagctca gcccatgaca aagaagcgaa aagtagatgg tttgagcaca gaggcagagc
  841 agcccttcat tccagtagag gtgctcgtag acctgttcct caaggaaggt gcctgtgatg
  901 aattgttctc ctacctcatt gagaaagtga agcgaaagaa aaatgtacta cgcctgtgct
  961 gtaagaagct gaagattttt gcaatgccca tgcaggatat caagatgatc ctgaaaatgg
1021 tgcagctgga ctctattgaa gatttggaag tgacttgtac ctggaagcta cccaccttgg
1081 cgaaattttc tccttacctg ggccagatga ttaatctgcg tagactcctc ctctcccaca
1141 tccatgcatc ttcctacatt tccccggaga aggaagagca gtatatcgcc cagttcacct
1201 ctcagttcct cagtctgcag tgcctgcagg ctctctatgt ggactcttta tttttcctta
1261 gaggccgcct ggatcagttg ctcaggcacg tgatgaaccc cttggaaacc ctctcaataa
1321 ctaactgccg gctttcggaa ggggatgtga tgcatctgtc ccagagtccc agcgtcagtc
1381 agctaagtgt cctgagtcta agtggggtca tgctgaccga tgtaagtccc gagcccctcc
1441 aagctctgct ggagagagcc tctgccaccc tccaggacct ggtctttgat gagtgtggga
1501 tcacggatga tcagctcctt gccctcctgc cttccctgag ccactgctcc cagcttacga
1561 ccttaagctt ctacgggaat tccatctcca tatctgccct gcagagtctc ctgcagcacc
1621 tcatcgggct gagcaatctg acccacgtgc tgtatcctgt ccccctggag agttatgagg
1681 acatccatgg taccctccac ctggagaggc ttgcctatct gcatgccagg ctcagggagt
1741 tgctgtgtga gttggggcgg cccagcatgg tctggcttag tgccaacccc tgtcctcact
1801 gtggggacag aaccttctat gacccggagc ccatcctgtg cccctgtttc atgcctaatt
1861 agctgggtgc acatatcaaa tgcttcattc tgcatacttg gacactaaag ccaggatgtg
1921 catgcatctt gaagcaacaa agcagccaca gtttcagaca aatgttcagt gtgagtgagg
1981 aaaacatgtt cagtgaggaa aaaacattca gacaaatgtt cagtgaggaa aaaaagggga
2041 agttgggggt aggcagatgt tgacttgagg agttaatgtg atctttgggg agatacatct
2101 tatagagtta gaaatagaat ctgaatttct aaagggagat tctggcttgg gaagtacatg
2161 taggagttaa tccctgtgta gactgttgta aagaaactgt tgaaaataaa gagaagcaat
2221 gtgaagca
Human PRAME isoform a (NP_006106.1; NP_996836.1; NP_996837.1; NP_996838.1;
NP_996839.1; NP_001278644.1; NP_001278645.1)
      1 merrrlwgsi qsryismsvw tsprrivela gqsilkdeal aiaalellpr elfpplfmaa
    61 fdgrhsqtlk amvqawpftc lplgvimkgq hihletfkav ldgldvllaq evrprrwklq
  121 vldirknshq dfwtvwsgnr aslysfpepe aaqpmtkkrk vdglsteaeg pfipvevlvd
  181 lflkegacde lfsyliekvk rkknvlrlcc kklkifampm qdikmilkmv qldsiedlev
  241 tctwklptla kfspylgqmi nlrrlllshi hassyispek eeqyiaqfts qflsiqclqa
  301 lyvdslfflr gridqlirhv mapletlsit norlsegdvm hisqspsysq lsvislsgvm
  361 ltdvspeplq allerasati qdlvidecgi tddqllallp sishcsqltt Isfygnsisi
  421 salgsllqhl iglsnlthvl ypvplesyed ihgtlhlerl aylharlrel lcelgrpsmv
  481 wlsanpcphc gdrtfydpep ilcpcfmpn
Human PRAME isoform b (NP_001278646.1; NP_001278648.1; NP_001305055.1;
NP_001305056.1)
      1 msvwtsprrl velagqsilk dealaiaale liprelfppl fmaafdgrhs qtlkamvqaw
    61 pftclplgvl mkgqhlhlet fkavldgldv llaqevrprr wklqvldirk nshqdfwtvw
  121 sgnraslysf pepeaaqpmt kkrkvdglst eaeqpfipve vlvdlflkeg acdelfsyli
  181 ekvkrkknvl rlcckklkif ampmqdikmi lkmvqldsie dlevtctwkl ptlakfspyl
  241 gqminirill lshihassyi spekeeqyia qftsqfislq ciqalyvdsl fflrgridql
  301 lrhvmnplet lsitncrlse gdvmhlsqsp svsqlsvlsl sgvmltdvsp eplgallera
  361 satlqdlvfd ecgitddqll allpsishcs qlttlsfygn sisisalqsl lqhliglsnl
  421 thvlypvple syedihgtlh lerlaylhar lrellcelgr psmvwlsanp cphcgdrtfy
  481 dpepilcpcf mpn
Representative Human HLA-A*02:01 DNA sequence
Atggccgtcatggcgccccgaaccctcgtcctgctactctcgggggctctggccctgacccagac
ctgggcgggctctcactccatgaggtatttcttcacatccgtgtcccggcccggccgcggggagc
cccgcttcatcgcagtgggctacgtggacgacacgcagttcgtgcggttcgacagcgacgccgcg
agccagaggatggagccgcgggcgccgtggatagagcaggagggtccggagtattgggacgggga
gacacggaaagtgaaggcccactcacagactcaccgagtggacctggggaccctgcgcggctact
acaaccagagcgaggccggttctcacaccgtccagaggatgtatggctgcgacgtggggtcggac
tggcgcttcctccgcgggtaccaccagtacgcctacgacggcaaggattacatcgccctgaaaga
ggacctgcgctcttggaccgcggcggacatggcagctcagaccaccaagcacaagtgggaggcgg
cccatgtggcggagcagttgagagcctacctggagggcacgtgcgtggagtggctccgcagatac
ctggagaacgggaaggagacgctgcagcgcacggacgcccccaaaacgcatatgactcaccacgc
tgtctctgaccatgaagccaccctgaggtgctgggccctgagcttctaccctgcggagatcacac
tgacctggcagcgggatggggaggaccagacccaggacacggagctcgtggagaccaggcctgca
ggggatggaaccttccagaagtgggcggctgtggtggtgccttctggacaggagcagagatacac
ctgccatgtgcagcatgagggtttgcccaagcccctcaccctgagatgggagccgtcttcccagc
ccaccatccccatcgtgggcatcattgctggcctggttctctttggagctgtgatcactggagct
gtggtcgctgctgtgatgtggaggaggaagagctcagatagaaaaggagggagctactctcaggc
tgcaagcagtgacagtgcccagggctctgatgtgtctctcacagcttgtaaagtgtga
Representative Human HLA-A*02:01 protein sequence
MAVMAPRTLVLLLSGALALTQTWAGSHSMRYFFTSVSRPGRGEPRFTAVGYVDDTQFVRFDSDAA
SQRMEPRAPWTEQEGPEYWDGETRKVKAHSQTHRVDLGTLRGYYNQSEAGSHTVQRMYGCDVGSD
WRFLRGYHQYAYDGKDYTALKEDLRSWTAADMAAQTTKHKWEAAHVABQLRAYLEGTCVEWLRRY
LENGKETLQRTDAPKTHMTHHAVSDHEATLRCWALSFYPAETTLTWQRDGEDQTQDTELVETRPA
GDGTFQKWAAVVVPSGQEQRYTCHVQHEGLPKPLTLRWEPSSQPTTPTVGTTAGLVLFGAVTTGA
VVAAVMWRRKSSDRKGGSYSQAASSDSAQGSDVSLTACKV*
Representative Vector
(the TCR-encoding protein of which can be interchanged with
any TCR sequence of interest): pTSLV102-MSCV-HA1-10-30-MGTM-Q-CD8
tggaagggctaattcactcccaaagaagacaagatatccttgatctgtggatctaccacacacaa
ggctacttccctgattagcagaactacacaccagggccaggggtcagatatccactgacctttgg
atggtgctacaagctagtaccagttgagccagataaggtagaagaggccaataaaggagagaaca
ccagcttgttacaccctgtgagcctgcatgggatggatgacccggagagagaagtgttagagtgg
aggtttgacagccgcctagcatttcatcacgtggcccgagagctgcatccggagtacttcaagaa
ctgctgatatcgagcttgctacaagggactttccgctggggactttccagggaggcgtggcctgg
ggggactggggagtggcgagccctcagatcctgcatataagcagctgctttttgcctgtactggg
tctctctggttagaccagatctgagcctgggagctctctggctaactagggaacccactgcttaa
gcctcaataaagcttgccttgagtgcttcaagtagtgtgtgcccgtctgttgtgtgactctggta
actagagatccctcagacccttttagtcagtgtggaaaatctctagcagtggcgcccgaacaggg
acttgaaagcgaaagggaaaccagaggagctctctcgacgcaggactcggcttgctgaagcgcgc
acggcaagaggcgaggggcggcgactggtgagtacgccaaaaattttgactagcggaggctagaa
ggagagagatgggtgcgagagcgtcagtattaagcgggggagaattagatcgcgatgggaaaaaa
ttcggttaaggccagggggaaagaaaaaatataaattaaaacatatagtatgggcaagcagggag
ctagaacgattcgcagttaatcctggcctgttagaaacatcagaaggctgtagacaaatactggg
acagctacaaccatcccttcagacaggatcagaagaacttagatcattatataatacagtagcaa
ccctctattgtgtgcatcaaaggatagagataaaagacaccaaggaagctttagacaagatagag
gaagagcaaaacaaaagtaagaccaccgcacagcaagcggccggccgctgatcttcagacctgga
ggaggagatatgagggacaattggagaagtgaattatataaatataaagtagtaaaaattgaacc
attaggagtagcacccaccaaggcaaagagaagagtggtgcagagagaaaaaagagcagtgggaa
taggagctttgttccttgggttcttgggagcagcaggaagcactatgggcgcagcgtcaatgacg
ctgacggtacaggccagacaattattgtctggtatagtgcagcagcagaacaatttgctgagggc
tattgaggcgcaacagcatctgttgcaactcacagtctggggcatcaagcagctccaggcaagaa
tcctggctgtggaaagatacctaaaggatcaacagctcctggggatttggggttgctctggaaaa
ctcatttgcaccactgctgtgccttggaatgctagttggagtaataaatctctggaacagatttg
gaatcacacgacctggatggagtgggacagagaaattaacaattacacaagcttaatacactcct
taattgaagaatcgcaaaaccagcaagaaaagaatgaacaagaattattggaattagataaatgg
gcaagtttgtggaattggtttaacataacaaattggctgtggtatataaaattattcataatgat
agtaggaggcttggtaggtttaagaatagtttttgctgtactttctatagtgaatagagttaggc
agggatattcaccattatcgtttcagacccacctcccaaccccgaggggacccgacaggcccgaa
ggaatagaagaagaaggtggagagagagacagagacagatccattcgattagtgaacggatctcg
acggtatcgccgaattaattcacaaatggcagtattcatccacaattttaaaagaaaagggggga
ttggggggtacagtgcaggggaaagaatagtagacataatagcaacagacatacaaactaaagaa
ttacaaaaacaaattacaaaaattcaaaattttcgggtttattacaggCGcGCcagagatccagt
ttggacCTgcAGGTGAAAGACCCCACCTGTAGGTTTGGCAAGTTAGCTTAAGTAACGCCATTTTG
CAAGGCATGGAAAATACATAACTGAGAATAGAGAAGTTCAGATCAAGGTTAGGAACAGAGAGACA
GCAGAATATGGGCCAAACAGGATATCTGTGGTAAGCAGTTCCTGCCCCGGCTCAGGGCCAAGAAC
AGATGGTCCCCAGATGCGGTCCCGCCCTCAGCAGTTTCTAGCGAACCATCAGATGTTTCCAGGGT
GCCCCAAGGACCTGAAATGACCCTGTGCCTTATTTGAACTAACCAATCAGTTTGCTTCTTGCTTC
TGTTTGTGTGCTTCTGCTCCCTGAGCTCAATAAAAGAGCCCACAACCCCTCACTTGGTGgGCCAG
TCCTCTGATAGACTGtGTCcCCTGGaTACCCGTAcggtaccgctagcgccaccATGGGCACCAGC
CTCCTCTGCTGGATGGCCCTGTGTCTCCTGGGGGCAGATCACGCAGATACTGGAGTCTCCCAGGA
CCCCAGACACAAGATCACAAAGAGGGGACAGAATGTTACTTTCAGGTGTGATCCAATTTCTGAAC
ACAACCGCCTTTATTGGTACCGCCAGACCCTGGGGCAGGGCCCAGAGTTTCTGACTTACTTCCAG
AATGAAGCTCAACTTGAAAAATCAAGGCTGCTCAGTGATCGGTTCTCTGCAGAGAGGCCTAAGGG
ATCTTTCTCCACCTTGGAGATCCAGCGCACAGAGCAGGGGGACTCTGCCATGTATCTCTGTGCCA
GCAGCCGCACTGCTGGAGATACTCAGTATTTTGGCCCAGGCACCCGGCTGACAGTGCTCGAAGAT
CTGAACAAGGTGTTCCCTCCAGAGGTGGCCGTGTTCGAGCCTTCTaAGGCCGAGATCgccCACAC
aCAGAAAGCCACCCTCGTGTGCCTGGCCACCGGCTTTTTCCCCGACCACGTGGAACTGTCTTGGT
GGGTCAACGGCAAAGAGGTGCACTCCGGCGTGTCAACgGATCCCCAGCCTCTGAAAGAACAGCCT
GCCCTGAACGACAGCCGGTACTGCCTGAGCTCCAGACTGAGAGTGTCCGCCACCTTCTGGCAGAA
CCCCCGGAACCACTTCAGATGCCAGGTGCAGTTTTACGGCCTGAGCGAGAACGACGAGTGGACCC
AGGACAGAGCCAAGCCCGTGACACAAATCGTGTCTGCCGAAGCCTGGGGAAGAGCCGATTGCGGC
ATCACCAGCGCCTCCTATCACCAGGGCGTGCTGAGCGCCACAATCCTGTACGAAATCCTGCTGGG
CAAGGCCACCCTGTACGCCGTGCTGGTGTCTGCTCTGGTGCTGATGGCCATGGTCAAGCGGAAGG
ACTTTGGCAGCGGCAGAGCCAAAAGGTCCGGGAGCGGTGCGACAAACTTTAGCCTGTTGAAACAA
GCCGGCGACGTTGAAGAGAACCCCGGACCTATGGAAACCCTcTTGGGCCTGCTTATCCTTTGGCT
GCAGCTGCAATGGGTGAGCAGCAAACAGGAGGTGACTCAGATTCCTGCAGCTCTGAGTGTCCCAG
AAGGAGAAAACTTGGTTCTCAACTGCAGTTTCACTGATAGCGCTATTTACAACCTCCAGTGGTTT
AGGCAGGACCCTGGGAAAGGCCTCACATCTCTGTTGCTTATTCAGTCAAGTCAGAGAGAGCAAAC
AAGTGGACGCCTTAATGCCTCTCTGGATAAATCATCAGGACGCAGTACTCTTTACATTGCAGCTT
CTCAGCCTGGTGATTCAGCCACCTACCTGTGCGCTGTGAGGGGTGGTACCTCAGGAACCTACAAA
TACATCTTTGGAACAGGCACCAGGCTGAAGGTTCTTGCAAACATCCAGAACCCCGACCCCGCCGT
GTACCAGCTGAGGGACTCCAAGTCCAGCGACAAGAGCGTGTGTCTGTTTACGGACTTCGACAGCC
AGACCAACGTGAGTCAAAGCAAGGACAGCGACGTCTACATAACGGATAAGACCGTGCTGGACATG
CGGAGCATGGACTTCAAGAGCAACAGCGCCGTGGCCTGGTCCAACAAGAGCGACTTCGCCTGCGC
CAACGCCTTCAACAACAGCATCATCCCCGAGGACACCTTCTTCCCCAGCAGCGACGTGCCCTGCG
ACGTGAAACTGGTGGAGAAGTCCTTCGAGACAGACACCAATCTGAACTTTCAGAACCTGCTGGTG
ATCGTGCTGCGGATTCTGCTGCTGAAAGTGGCCGGCTTCAATCTGCTGATGACCCTGCGGCTGTG
GAGCAGCAGGGCTAAGAGGTCCGGCAGCGGAGCCACCAATTTTTCCCTGCTGAAACAGGCTGGTG
ACGTGGAAGAAAACCCTGGCCCCATGGCGCTGCCCGTCACCGCGCTGCTGCTGCCCCTGGCGCTG
CTGTTACACGCCGCTCGGCCAGAGCTTCCCACCCAGGGCACATTCTCCAACGTGTCCACCAATGT
GTCGGGAGGCGGCGGATCGTCCCAGTTCAGAGTGTCCCCTCTGGACCGCACCTGGAACCTGGGCG
AGACCGTGGAGCTGAAATGTCAGGTCCTGCTGAGCAACCCGACCTCCGGGTGCAGTTGGCTGTTC
CAGCCGCGTGGTGCTGCCGCAAGCCCTACGTTCCTGCTTTACCTGAGCCAGAACAAGCCCAAGGC
GGCCGAGGGCCTGGACACCCAGAGATTCTCCGGCAAGCGCCTGGGGGACACATTCGTGCTTACTT
TGAGCGATTTCCGCAGAGAGAACGAGGGCTACTATTTCTGTTCGGCGCTGAGCAATTCCATCATG
TATTTCAGCCACTTTGTGCCAGTGTTCCTGCCTGCCAAGCCTACCACAACACCAGCTCCCCGTCC
CCCGACTCCGGCGCCTACCATCGCGAGTCAACCGTTGAGCCTGAGGCCTGAGGCTTGTCGGCCCG
CTGCGGGGGGTGCCGTCCACACCAGGGGCCTCGACTTTGCGTGCGACATCTATATTTGGGCGCCT
CTGGCGGGTACCTGCGGGGTGCTGCTGCTGTCATTGGTGATTACCCTGTACTGCAATCACCGCAA
CCGCCGGGGGTCTGTAAGTGCCCACGGCCTGTGGTCAAGTCCGGTGACAAACCGTCGCTCTCGGC
TCGCTACGTGCGCGCTAAGCGCAGCGGTTCCGGGGCCACCAACTTTTCATTGCTGAAGCAGGCCG
GTGATGTGGAGGAGAATCCAGGGCCCATGCGCCCCAGGCTTTGGCTCCTTCTTGCTGCTCAGCTC
ACTGTCTTGCATGGCAACTCCGTTCTGCAGCAGACTCCCGCCTACATCAAGGTGCAGACGAACAA
GATGGTGATGCTGTCATGCGAGGCCAAGATCTCTCTTTCAAATATGAGAATTTATTGGCTACGAC
AGCGCCAGGCCCCCTCCAGCGACAGCCACCACGAGTTCCTGGCGCTTTGGGATTCTGCTAAAGGC
ACCATCCATGGAGAGGAGGTGGAACAGGAGAAGATAGCTGTCTTCCGCGACGCATCCCGCTTCAT
CCTGAACCTGACCAGCGTGAAGCCGGAGGACAGCGGCATCTACTTCTGTATGATCGTTGGCTCCC
CCGAGCTGACCTTCGGCAAAGGCACCCAGCTGTCCGTGGTGGACTTCCTGCCCACCACAGCCCAG
CCAACCAAGAAATCCACCCTCAAGAAGCGCGTGTGCCGACTGCCCCGCCCTGAAACCCAGAAGGG
CCCTCTGTGCTCCCCCATCACCCTTGGACTGCTGGTGGCGGGAGTCCTGGTGCTGCTCGTATCTC
TGGGTGTCGCCATCCACCTGTGCTGCCGCCGCCGCCGCGCCCGCCTGAGGTTTATGAAACAGTTT
TACAAGTGATAAatcgatagatcctaatcaacctctggattacaaaatttgtgaaagattgactg
gtattcttaactatgttgctccttttacgctatgtggatacgctgctttaatgcctttgtatcat
gctattgcttcccgtatggctttcattttctcctccttgtataaatcctggttgctgtctcttta
tgaggagttgtggcccgttgtcaggcaacgtggcgtggtgtgcactgtgtttgctgacgcaaccc
ccactggttggggcattgccaccacctgtcagctcctttccgggactttcgctttccccctccct
attgccacggcggaactcatcgccgcctgccttgcccgctgctggacaggggctcggctgttggg
cactgacaattccgtggtgttgtcggggaaatcatcgtcctttccttggctgctcgcctgtgttg
ccacctggattctgcgcgggacgtccttctgctacgtcccttcggccctcaatccagcggacctt
ccttcccgcggcctgctgccggctctgcggcctcttccgcgtcttcgccttcgccctcagacgag
tcggatctccctttgggccgcctccccgcctgagatcctttaagaccaatgacttacaaggcagc
tgtagatcttagccactttttaaaagaaaaggggggactggaagggctaattcactcccaacgaa
gacaagatctgctttttgcttgtactgggtctctctggttagaccagatctgagcctgggagctc
tctggctaactagggaacccactgcttaagcctcaataaagcttgccttgagtgcttcaagtagt
gtgtgcccgtctgttgtgtgactctggtaactagagatccctcagacccttttagtcagtgtgga
aaatctctagcagtagtagttcatgtcatcttattattcagtatttataacttgcaaagaaatga
atatcagagagtgagaggcccgggttaattaaggaaagggctagatcattcttgaagacgaaagg
gcctcgtgatacgcctatttttataggttaatgtcatgataataatggtttcttagacgtcaggt
ggcacttttcggggaaatgtgcgcggaacccctatttgtttatttttctaaatacattcaaatat
gtatccgctcatgagacaataaccctgataaatgcttcaataatattgaaaaaggaagagtatga
gtattcaacatttccgtgtcgcccttattcccttttttgcggcattttgccttcctgtttttgct
cacccagaaacgctggtgaaagtaaaagatgctgaagatcagttgggtgcacgagtgggttacat
cgaactggatctcaacagcggtaagatccttgagagttttcgccccgaagaacgttttccaatga
tgagcacttttaaagttctgctatgtggcgcggtattatcccgtgttgacgccgggcaagagcaa
ctcggtcgccgcatacactattctcagaatgacttggttgagtactcaccagtcacagaaaagca
tcttacggatggcatgacagtaagagaattatgcagtgctgccataaccatgagtgataacactg
cggccaacttacttctgacaacgatcggaggaccgaaggagctaaccgcttttttgcacaacatg
ggggatcatgtaactcgccttgatcgttgggaaccggagctgaatgaagccataccaaacgacga
gcgtgacaccacgatgcctgtagcaatggcaacaacgttgcgcaaactattaactggcgaactac
ttactctagcttcccggcaacaattaatagactggatggaggcggataaagttgcaggaccactt
ctgcgctcggcccttccggctggctggtttattgctgataaatctggagccggtgagcgtgggtc
tcgcggtatcattgcagcactggggccagatggtaagccctcccgtatcgtagttatctacacga
cggggagtcaggcaactatggatgaacgaaatagacagatcgctgagataggtgcctcactgatt
aagcattggtaactgtcagaccaagtttactcatatatactttagattgatttaaaacttcattt
ttaatttaaaaggatctaggtgaagatcctttttgataatctcatgaccaaaatcccttaacgtg
agttttcgttccactgagcgtcagaccccgtagaaaagatcaaaggatcttcttgagatcctttt
tttctgcgcgtaatctgctgcttgcaaacaaaaaaaccaccgctaccagcggtggtttgtttgcc
ggatcaagagctaccaactctttttccgaaggtaactggcttcagcagagcgcagataccaaata
ctgttcttctagtgtagccgtagttaggccaccacttcaagaactctgtagcaccgcctacatac
ctcgctctgctaatcctgttaccagtggctgctgccagtggcgataagtcgtgtcttaccgggtt
ggactcaagacgatagttaccggataaggcgcagcggtcgggctgaacggggggttcgtgcacac
agcccagcttggagcgaacgacctacaccgaactgagatacctacagcgtgagctatgagaaagc
gccacgcttcccgaagggagaaaggcggacaggtatccggtaagcggcagggtcggaacaggaga
gcgcacgagggagcttccagggggaaacgcctggtatctttatagtcctgtcgggtttcgccacc
tctgacttgagcgtcgatttttgtgatgctcgtcaggggggcggagcctatggaaaaacgccagc
aacgcggcctttttacggttcctggccttttgctggccttttgctcacatgttctttcctgcgtt
atccCCTGATTCTGTGGATAACCGTATTACCGCCTTTGAGTGAGCTGATACCGCTCGCCGCAGCC
GAACGACCGAGCGCAGCGAGTCAGTGAGCGAGGAAGCGGAAGAGCGCCCAATACGCAAACCGCCT
CTCCCCGCGCGTTGGCCGATTCATTAATGCAGCAAGCTCATGGCTGACTAATTTTTTTTATTTAT
GCAGAGGCCGAGGCCGCCTCGGCCTCTGAGCTATTCCAGAAGTAGTGAGGAGGCTTTTTTGGAGG
CCTAGGCTTTTGCAAAAAGCTCCCCGTGGCACGACAGGTTTCCCGACTGGAAAGCGGGCAGTGAG
CGCAACGCAATTAATGTGAGTTAGCTCACTCATTAGGCACCCCAGGCTTTACACTTTATGCTTCC
GGCTCGTATGTTGTGTGGAATTGTGAGCGGATAACAATTTCACACAGGAAACAGCTATGACATGA
TTACGAATTTCACAAATAAAGCATTTTTTTCACTGCATTCTAGTTGTGGTTTGTCCAAACTCATC
AATGTATCTTATCATGTCTGGATCAACTGGATAACTCAAGCTAACCAAAATCATCCCAAACTTCC
CACCCCATACCCTATTACCACTGCCAATTACCTGTGGTTTCATTTACTCTAAACCTGTGATTCCT
CTGAATTATTTTCATTTTAAAGAAATTGTATTTGTTAAATATGTACTACAAACTtagtagt
Representative Vector
(the TCR-encoding protein of which can be interchanged with
any TCR sequence of interest): pHAGE-MSCV-HN-P32-41-P2A-dnTGFbRII
(with dnTGFbRII highlighted in bold text)
tggaagggctaattcactcccaaagaagacaagatatccttgatctgtggatctaccacacacaa
ggctacttccctgattagcagaactacacaccagggccaggggtcagatatccactgacctttgg
atggtgctacaagctagtaccagttgagccagataaggtagaagaggccaataaaggagagaaca
ccagcttgttacaccctgtgagcctgcatgggatggatgacccggagagagaagtgttagagtgg
aggtttgacagccgcctagcatttcatcacgtggcccgagagctgcatccggagtacttcaagaa
ctgctgatatcgagcttgctacaagggactttccgctggggactttccagggaggcgtggcctgg
ggggactggggagtggcgagccctcagatcctgcatataagcagctgctttttgcctgtactggg
tctctctggttagaccagatctgagcctgggagctctctggctaactagggaacccactgcttaa
gcctcaataaagcttgccttgagtgcttcaagtagtgtgtgcccgtctgttgtgtgactctggta
actagagatccctcagacccttttagtcagtgtggaaaatctctagcagtggcgcccgaacaggg
acttgaaagcgaaagggaaaccagaggagctctctcgacgcaggactcggcttgctgaagcgcgc
acggcaagaggcgaggggcggcgactggtgagtacgccaaaaattttgactagcggaggctagaa
ggagagagatgggtgcgagagcgtcagtattaagcgggggagaattagatcgcgatgggaaaaaa
ttcggttaaggccagggggaaagaaaaaatataaattaaaacatatagtatgggcaagcagggag
ctagaacgattcgcagttaatcctggcctgttagaaacatcagaaggctgtagacaaatactggg
acagctacaaccatcccttcagacaggatcagaagaacttagatcattatataatacagtagcaa
ccctctattgtgtgcatcaaaggatagagataaaagacaccaaggaagctttagacaagatagag
gaagagcaaaaaaaagtaagaccaccgcacagcaagcggccggccgctgatcttcagacctggag
gaggagatatgagggacaattggagaagtgaattatataaatataaagtagtaaaaattgaacca
ttaggagtagcacccaccaaggcaaagagaagagtggtgcagagagaaaaaagagcagtgggaat
aggagctttgttccttgggttcttgggagcagcaggaagcactatgggcgcagcgtcaatgacgc
tgacggtacaggccagacaattattgtctggtatagtgcagcagcagaacaatttgctgagggct
attgaggcgcaacagcatctgttgcaactcacagtctggggcatcaagcagctccaggcaagaat
cctggctgtggaaagatacctaaaggatcaacagctcctggggatttggggttgctctggaaaac
tcatttgcaccactgctgtgccttggaatgctagttggagtaataaatctctggaacagatttgg
aatcacacgacctggatggagtgggacagagaaattaacaattacacaagcttaatacactcctt
aattgaagaatcgcaaaaccagcaagaaaagaatgaacaagaattattggaattagataaatggg
caagtttgtggaattggtttaacataacaaattggctgtggtatataaaattattcataatgata
gtaggaggcttggtaggtttaagaatagtttttgctgtactttctatagtgaatagagttaggca
gggatattcaccattatcgtttcagacccacctcccaaccccgaggggacccgacaggcccgaag
gaatagaagaagaaggtggagagagagacagagacagatccattcgattagtgaacggatctcga
cggtatcgccgaattaattcacaaatggcagtattcatccacaattttaaaagaaaaggggggat
tggggggtacagtgcaggggaaagaatagtagacataatagcaacagacatacaaactaaagaat
tacaaaaacaaattacaaaaattcaaaattttcgggtttattacaggCGcGCcagagatccagtt
tggacCTgcAGGTGAAAGACCCCACCTGTAGGTTTGGCAAGTTAGCTTAAGTAACGCCATTTTGC
AAGGCATGGAAAATACATAACTGAGAATAGAGAAGTTCAGATCAAGGTTAGGAACAGAGAGACAG
CAGAATATGGGCCAAACAGGATATCTGTGGTAAGCAGTTCCTGCCCCGGCTCAGGGCCAAGAACA
GATGGTCCCCAGATGCGGTCCCGCCCTCAGCAGTTTCTAGCGAACCATCAGATGTTTCCAGGGTG
CCCCAAGGACCTGAAATGACCCTGTGCCTTATTTGAACTAACCAATCAGTTtGCTTCTtGCTTCT
GTTtGtGtGCTTCTGCTCCCTGAGCTCAATAAAAGAGCCCACAACCCCTCACTTGGtGgGCCAGT
CCTCtGATAGACTGtGTCcCCtGGaTACCCGTAcggtaccgctagcgccaccATGGGCTCCTGGA
CCCTCTGCTGTGTGTCCCTTTGCATCCTGGTTGCAAAGCACACAGATGCTGGAGTTATCCAGTCA
CCCCGGCACGAGGTGACAGAGATGGGACAAGAAGTGACTCTGAGATGTAAACCAATTTCAGGACA
TGACTACCTTTTCTGGTACAGACAGACCATGATGCGGGGACTGGAGTTGCTCATTTACTTTAACA
ACAACGTTCCTATTGATGATTCAGGGATGCCCGAGGATCGCTTCTCAGCTAAGATGCCTAATGCA
TCATTCTCCACTCTGAAGATCCAGCCCTCAGAACCCAGGGACTCAGCTGTGTACTTCTGTGCCAG
CAGTTTTCTCGGCTGGAATGAAAAACTGTTCTTTGGCAGTGGAACCCAGCTCTCTGTCTTGGAAG
ATCTGAACAAGGTGTTCCCTCCAGAGGTGGCCGTGTTCGAGCCTTCTaAGGCCGAGATCgccCAC
ACaCAaAAAGCCACCCTCGTGTGCCTGGCCACCGGCTTTTTCCCCGACCACGTGGAACTGTCTTG
GTGGGTCAACGGCAAAGAGGTGCACTCCGGCGTGtcAACgGATCCCCAGCCTCTGAAAGAACAGC
CTGCCCTGAACGACAGCCGGTACTGCCTGAGCTCCAGACTGAGAGTGTCCGCCACCTTCTGGCAG
AACCCCCGGAACCACTTCAGATGCCAGGTGCAGTTTTACGGCCTGAGCGAGAACGACGAGTGGAC
CCAGGACAGAGCCAAGCCCGTGACACAAATCGTGTCTGCCGAAGCCTGGGGAAGAGCCGATTGCG
GCATCACCAGCGCCTCCTATCACCAGGGCGTGCTGAGCGCCACAATCCTGTACGAAATCCTGCTG
GGCAAGGCCACCCTGTACGCCGTGCTGGTGTCTGCTCTGGTGCTGATGGCCATGGTCAAGCGGAA
GGACTTTGGCAGCGGCAGAGCCAAAAGGTCCGGGAGCGGTGCGACAAACTTTAGCCTGTTGAAAC
AAGCCGGCGACGTTGAAGAGAACCCCGGACCTATGGTCCTGAAATTCTCCGTGTCCATTCTTTGG
ATTCAGTTGGCATGGGTGAGCACCCAGCTGCTGGAGCAGAGCCCTCAGTTTCTTAGCATCCAAGA
GGGAGAAAATCTCACTGTGTACTGCAACTCCTCAAGTGTTTTCTCCAGCCTTCAATGGTACAGAC
AGGAGCCTGGGGAAGGTCCTGTCCTCCTGGTGACAGTTGTTACTGGTGGAGAAGTGAAGAAGCTG
AAGAGACTTACCTTTCAGTTTGGTGATGCAAGAAAGGACAGTTCTCTCCACATCACTGCAGCCCA
GCCTGGTGATACAGGCCTCTACCTCTGTGCAGGAGATGAAAGTATTAGCTATGGAAAGCTGACAT
TTGGACAAGGGACCATCTTGACTGTCCATCCAAacatccagaaccccgaccccgccgtgtaccag
ctgagggactccaagtccagcgacaagagcgtgtgtctgtttacggacttcgacagccagaccaa
cgtgagtcaaagcaaggacagcgacgtctacataacggataagaccgtgctggacatgcggagca
tggacttcaagagcaacagcgccgtggcctggtccaacaagagcgacttcgcctgcgccaacgcc
ttcaacaacagcatcatccccgaggacaccttcttccccagcagcgacgtgccctgcgacgtgaa
actggtggagaagtccttcgagacagacaccaatctgaactttcagaacctgctggtgatcgtgc
tgcggattctgctgCTGAAAGTGGCCGGCTTCAATCTGCTGATGACCCTGCGGCTGTGGAGCAGC
AGGGCTAAGAGGTCCGGCAGCGGAGCCACCAATTTTTCCCTGCTGAAACAGGCTGGTGACGTGGA
AGAAAACCCTGGCCCCATGGGTCGGGGGCTGCTCAGGGGCCTGTGGCCGCTGCACATCGTCCTGT
GGACGCGTATCGCCAGCACGATCCCACCGCACGTTCAGAAGTCGGTTAATAACGACATGATAGTC
ACTGACAACAACGGTGCAGTCAAGTTTCCACAACTGTGTAAATTTTGTGATGTGAGATTTTCCAC
CTGTGACAACCAGAAATCCTGCATGAGCAACTGCAGCATCACCTCCATCTGTGAGAAGCCACAGG
AAGTCTGTGTGGCTGTATGGAGAAAGAATGACGAGAACATAACACTAGAGACAGTTTGCCATGAC
CCCAAGCTCCCCTACCATGACTTTATTCTGGAAGATGCTGCTTCTCCAAAGTGCATTATGAAGGA
AAAAAAAAAGCCTGGTGAGACTTTCTTCATGTGTTCCTGTAGCTCTGATGAGTGCAATGACAACA
TCATCTTCTCAGAAGAATATAACACCAGCAATCCTGACTTGTTGCTAGTCATATTTCAAGTGACA
GGCATCAGCCTCCTGCCACCACTGGGAGTTGCCATATCTGTCATCATCATCTTCTACTGCTACCG
CGTTaaccggcagcagaagTAGTGATAAatcgatagatcctaatcaacctctggattacaaaatt
tgtgaaagattgactggtattcttaactatgttgctccttttacgctatgtggatacgctgcttt
aatgcctttgtatcatgctattgcttcccgtatggctttcattttctcctccttgtataaatcct
ggttgctgtctctttatgaggagttgtggcccgttgtcaggcaacgtggcgtggtgtgcactgtg
tttgctgacgcaacccccactggttggggcattgccaccacctgtcagctcctttccgggacttt
cgctttccccctccctattgccacggcggaactcatcgccgcctgccttgcccgctgctggacag
gggctcggctgttgggcactgacaattccgtggtgttgtcggggaaatcatcgtcctttccttgg
ctgctcgcctgtgttgccacctggattctgcgcgggacgtccttctgctacgtcccttcggccct
caatccagcggaccttccttcccgcggcctgctgccggctctgcggcctcttccgcgtcttcgcc
ttcgccctcagacgagtcggatctccctttgggccgcctccccgcctgagatcctttaagaccaa
tgacttacaaggcagctgtagatcttagccactttttaaaagaaaaggggggactggaagggcta
attcactcccaacgaagacaagatctgctttttgcttgtactgggtctctctggttagaccagat
ctgagcctgggagctctctggctaactagggaacccactgcttaagcctcaataaagcttgcctt
gagtgcttcaagtagtgtgtgcccgtctgttgtgtgactctggtaactagagatccctcagaccc
ttttagtcagtgtggaaaatctctagcagtagtagttcatgtcatcttattattcagtatttata
acttgcaaagaaatgaatatcagagagtgagaggcccgggttaattaaggaaagggctagatcat
tcttgaagacgaaagggcctcgtgatacgcctatttttataggttaatgtcatgataataatggt
ttcttagacgtcaggtggcacttttcggggaaatgtgcgcggaacccctatttgtttatttttct
aaatacattcaaatatgtatccgctcatgagacaataaccctgataaatgcttcaataatattga
aaaaggaagagtatgagtattcaacatttccgtgtcgcccttattcccttttttgcggcattttg
ccttcctgtttttgctcacccagaaacgctggtgaaagtaaaagatgctgaagatcagttgggtg
cacgagtgggttacatcgaactggatctcaacagcggtaagatccttgagagttttcgccccgaa
gaacgttttccaatgatgagcacttttaaagttctgctatgtggcgcggtattatcccgtgttga
cgccgggcaagagcaactcggtcgccgcatacactattctcagaatgacttggttgagtactcac
cagtcacagaaaagcatcttacggatggcatgacagtaagagaattatgcagtgctgccataacc
atgagtgataacactgcggccaacttacttctgacaacgatcggaggaccgaaggagctaaccgc
ttttttgcacaacatgggggatcatgtaactcgccttgatcgttgggaaccggagctgaatgaag
ccataccaaacgacgagcgtgacaccacgatgcctgtagcaatggcaacaacgttgcgcaaacta
ttaactggcgaactacttactctagcttcccggcaacaattaatagactggatggaggcggataa
agttgcaggaccacttctgcgctcggcccttccggctggctggtttattgctgataaatctggag
ccggtgagcgtgggtctcgcggtatcattgcagcactggggccagatggtaagccctcccgtatc
gtagttatctacacgacggggagtcaggcaactatggatgaacgaaatagacagatcgctgagat
aggtgcctcactgattaagcattggtaactgtcagaccaagtttactcatatatactttagattg
atttaaaacttcatttttaatttaaaaggatctaggtgaagatcctttttgataatctcatgacc
aaaatcccttaacgtgagttttcgttccactgagcgtcagaccccgtagaaaagatcaaaggatc
ttcttgagatcctttttttctgcgcgtaatctgctgcttgcaaacaaaaaaaccaccgctaccag
cggtggtttgtttgccggatcaagagctaccaactctttttccgaaggtaactggcttcagcaga
gcgcagataccaaatactgttcttctagtgtagccgtagttaggccaccacttcaagaactctgt
agcaccgcctacatacctcgctctgctaatcctgttaccagtggctgctgccagtggcgataagt
cgtgtcttaccgggttggactcaagacgatagttaccggataaggcgcagcggtcgggctgaacg
gggggttcgtgcacacagcccagcttggagcgaacgacctacaccgaactgagatacctacagcg
tgagctatgagaaagcgccacgcttcccgaagggagaaaggcggacaggtatccggtaagcggca
gggtcggaacaggagagcgcacgagggagcttccagggggaaacgcctggtatctttatagtcct
gtcgggtttcgccacctctgacttgagcgtcgatttttgtgatgctcgtcaggggggcggagcct
atggaaaaacgccagcaacgcggcctttttacggttcctggccttttgctggccttttgctcaca
tgttctttcctgcgttatccCCTGATTCTGTGGATAACCGTATTACCGCCTTTGAGTGAGCTGAT
ACCGCTCGCCGCAGCCGAACGACCGAGCGCAGCGAGTCAGTGAGCGAGGAAGCGGAAGAGCGCCC
AATACGCAAACCGCCTCTCCCCGCGCGTTGGCCGATTCATTAATGCAGCAAGCTCATGGCTGACT
AATTTTTTTTATTTATGCAGAGGCCGAGGCCGCCTCGGCCTCTGAGCTATTCCAGAAGTAGTGAG
GAGGCTTTTTTGGAGGCCTAGGCTTTTGCAAAAAGCTCCCCGTGGCACGACAGGTTTCCCGACTG
GAAAGCGGGCAGTGAGCGCAACGCAATTAATGTGAGTTAGCTCACTCATTAGGCACCCCAGGCTT
TACACTTTATGCTTCCGGCTCGTATGTTGTGTGGAATTGTGAGCGGATAACAATTTCACACAGGA
AACAGCTATGACATGATTACGAATTTCACAAATAAAGCATTTTTTTCACTGCATTCTAGTTGTGG
TTTGTCCAAACTCATCAATGTATCTTATCATGTCTGGATCAACTGGATAACTCAAGCTAACCAAA
ATCATCCCAAACTTCCCACCCCATACCCTATTACCACTGCCAATTACCTGTGGTTTCATTTACTC
TAAACCTGTGATTCCTCTGAATTATTTTCATTTTAAAGAAATTGTATTTGTTAAATATGTACTAC
AAACTtagtagt
```

TABLE 4
R11P3D3 TCR MGTM codon optimized sequence (used in working
examples herein as “Comparator TCR”)
Alpha chain:
TRAV24/TRAJ43/MGTM modified TRAC
Alpha chain DNA sequence
ATGGAGAAGAATCCTTTGGCAGCCCCATTACTAATCCTCTGGTTTCATCTTGACT
GCGTGAGCAGCATACTGAACGTGGAACAAAGTCCTCAGTCACTGCATGTTCAGGA
GGGAGACAGCACCAATTTCACCTGCAGCTTCCCTTCCAGCAATTTTTATGCCTTA
CACTGGTACAGATGGGAAACTGCAAAAAGCCCCGAGGCCTTGTTTGTAATGACTT
TAAATGGGGATGAAAAGAAGAAAGGACGAATAAGTGCCACTCTTAATACCAAGGA
GGGTTACAGCTATTTGTACATCAAAGGATCCCAGCCTGAAGACTCAGCCACATAC
CTCTGTGCCCTGTACAATAACAATGACATGCGCTTTGGAGCAGGGACCAGACTGA
CAGTAAAACCAAacatccagaaccccgaccccgccgtgtaccagctgagggactc
caagtccagcgacaagagcgtgtgtctgtttacggacttcgacagccagaccaac
gtgagtcaaagcaaggacagcgacgtctacataacggataagaccgtgctggaca
tgcggagcatggacttcaagagcaacagcgccgtggcctggtccaacaagagcga
cttcgcctgcgccaacgccttcaacaacagcatcatccccgaggacaccttcttc
cccagcagcgacgtgccctgcgacgtgaaactggtggagaagtccttcgagacag
acaccaatctgaactttcagaacctgctggtgatcgtgctgcggattctgctgct
gaaagtggccggcttcaatctgctgatgaccctgcggctgtggagc
Alpha chain protein sequence
MEKNPLAAPLLILWFHLDCVSSILNVEQSPQSLHVQEGDSTNFTCSFPSSNFYAL
HWYRWETAKSPEALFVMTLNGDEKKKGRISATLNTKEGYSYLYIKGSQPEDSATY
LCALYNNNDMRFGAGTRLTVKPNiqnpdpavyqlrdskssdksvclftdfdsqtn
vsqskdsdvyitdktvldmrsmdfksnsavawsnksdfacanafnnsiipedtff
pssdvpcdvklveksfetdtnlnfqnllvivlrilllkvagfnllmtlrlws
Beta chain:
TRBV12-3/TRBJ2-3/MGTM modified TRBC
Beta chain DNA sequence
ATGGACTCCTGGACCTTCTGCTGTGTGTCCCTTTGCATCCTGGTAGCGAAGCATA
CAGATGCTGGAGTTATCCAGTCACCCCGCCATGAGGTGACAGAGATGGGACAAGA
AGTGACTCTGAGATGTAAACCAATTTCAGGCCACAACTCCCTTTTCTGGTACAGA
CAGACCATGATGCGGGGACTGGAGTTGCTCATTTACTTTAACAACAACGTTCCGA
TAGATGATTCAGGGATGCCCGAGGATCGATTCTCAGCTAAGATGCCTAATGCATC
ATTCTCCACTCTGAAGATCCAGCCCTCAGAACCCAGGGACTCAGCTGTGTACTTC
TGTGCCAGCAGTCCCGGCAGCACAGATACGCAGTATTTTGGCCCAGGCACCCGGC
TGACAGTGCTCGaagatctgaacaaggtgttccctccagaggtggccgtgttcga
gccttctaaggccgagatcgcccacacacaaaaagccaccctcgtgtgcctggcc
accggctttttccccgaccacgtggaactgtcttggtgggtcaacggcaaagagg
tgcactccggcgtgtcaacggatccccagcctctgaaagaacagcctgccctgaa
cgacagccggtactgcctgagctccagactgagagtgtccgccaccttctggcag
aacccccggaaccacttcagatgccaggtgcagttttacggcctgagcgagaacg
acgagtggacccaggacagagccaagcccgtgacacaaatcgtgtctgccgaagc
ctggggaagagccgattgcggcatcaccagcgcctcctatcaccagggcgtgctg
agcgccacaatcctgtacgaaatcctgctgggcaaggccaccctgtacgccgtgc
tggtgtctgctctggtgctgatggccatggtcaagcggaaggactttggcagcgg
cagagccaaaaggtccgggagcggt
Beta chain protein sequence
MDSWTFCCVSLCILVAKHTDAGVIQSPRHEVTEMGQEVTLRCKPISGHNSLFWYR
QTMMRGLELLIYFNNNVPIDDSGMPEDRFSAKMPNASESTLKIQPSEPRDSAVYF
CASSPGSTDTQYFGPGTRLTVLEdlnkvfppevavfepskaeiahtqkatlvcla
tgffpdhvelswwvngkevhsgvstdpqplkeqpalndsryclssrlrvsatfwq
nprnhfreqvqfyglsendewtqdrakpvtqivsaeawgradcgitsasyhqgvl
satilyeillgkatlyavlvsalvlmamvkrkdfgsgrakrsgsg
Complete Beta and Alpha ORF DNA Sequence (The underlined
italic region in the “Furin-P2A″ site encodes a sequence
allowing for expression of two polypeptide chains in a
single cassette)
ATGGACTCCTGGACCTTCTGCTGTGTGTCCCTTTGCATCCTGGTAGCGAAGCATA
CAGATGCTGGAGTTATCCAGTCACCCCGCCATGAGGTGACAGAGATGGGACAAGA
AGTGACTCTGAGATGTAAACCAATTTCAGGCCACAACTCCCTTTTCTGGTACAGA
CAGACCATGATGCGGGGACTGGAGTTGCTCATTTACTTTAACAACAACGTTCCGA
TAGATGATTCAGGGATGCCCGAGGATCGATTCTCAGCTAAGATGCCTAATGCATC
ATTCTCCACTCTGAAGATCCAGCCCTCAGAACCCAGGGACTCAGCTGTGTACTTC
TGTGCCAGCAGTCCCGGCAGCACAGATACGCAGTATTTTGGCCCAGGCACCCGGC
TGACAGTGCTCGaagatctgaacaaggtgttccctccagaggtggccgtgttcga
gccttctaaggccgagatcgcccacacacaaaaagccaccctcgtgtgcctggcc
accggctttttccccgaccacgtggaactgtcttggtgggtcaacggcaaagagg
tgcactccggcgtgtcaacggatccccagcctctgaaagaacagcctgccctgaa
cgacagccggtactgcctgagctccagactgagagtgtccgccaccttctggcag
aacccccggaaccacttcagatgccaggtgcagttttacggcctgagcgagaacg
acgagtggacccaggacagagccaagcccgtgacacaaatcgtgtctgccgaagc
ctggggaagagccgattgcggcatcaccagcgcctcctatcaccagggcgtgctg
agcgccacaatcctgtacgaaatcctgctgggcaaggccaccctgtacgccgtgc
tggtgtctgctctggtgctgatggccatggtcaagcggaaggactttggcagagg
cagagccaaaaggtccgggagcggtGCGACAAACTTTAGCCTGTTGAAACAAGCC
GGCGACGTTGAAGAGAACCCCGGACCTATGGAGAAGAATCCTTTGGCAGCCCCAT
TACTAATCCTCTGGTTTCATCTTGACTGCGTGAGCAGCATACTGAACGTGGAACA
AAGTCCTCAGTCACTGCATGTTCAGGAGGGAGACAGCACCAATTTCACCTGCAGC
TTCCCTTCCAGCAATTTTTATGCCTTACACTGGTACAGATGGGAAACTGCAAAAA
GCCCCGAGGCCTTGTTTGTAATGACTTTAAATGGGGATGAAAAGAAGAAAGGACG
AATAAGTGCCACTCTTAATACCAAGGAGGGTTACAGCTATTTGTACATCAAAGGA
TCCCAGCCTGAAGACTCAGCCACATACCTCTGTGCCCTGTACAATAACAATGACA
TGCGCTTTGGAGCAGGGACCAGACTGACAGTAAAACCAAacatccagaaccccga
ccccgccgtgtaccagctgagggactccaagtccagcgacaagagcgtgtgtctg
tttacggacttcgacagccagaccaacgtgagtcaaagcaaggacagcgacgtct
acataacggataagaccgtgctggacatgcggagcatggacttcaagagcaacag
cgccgtggcctggtccaacaagagcgacttcgcctgcgccaacgccttcaacaac
agcatcatccccgaggacaccttcttccccagcagcgacgtgccctgcgacgtga
aactggtggagaagtccttcgagacagacaccaatctgaactttcagaacctgct
ggtgatcgtgctgcggattctgctgctgaaagtggccggcttcaatctgctgatg
accctgcggctgtggagc
Complete Beta and Alpha ORF Protein Sequence
(The underlined italic region in the “Furin-
P2A” site allows expression of two polypeptide
chains in a single cassette)
MDSWTFCCVSLCILVAKHTDAGVIQSPRHEVTEMGQEVTLRCKPISGHNSLFWYR
QTMMRGLELLIYFNNNVPIDDSGMPEDRFSAKMPNASFSTLKIQPSEPRDSAVYF
CASSPGSTDTQYFGPGTRLTVLEdlnkvfppevavfepskaeiahtqkatlvcla
tgffpdhvelswwvngkevhsgvstdpqplkeqpalndsryclssrlrvsatfwq
nprnhfreqvqfyglsendewtqdrakpvtqivsaeawgradcgitsasyhqgvl
satilyeillgkatlyavlvsalvlmamvkrkdfgsgrakrsgsgATNFSLLKQA
GDVEENPGPMEKNPLAAPLLILWFHLDCVSSILNVEQSPQSLHVQEGDSTNFTCS
FPSSNFYALHWYRWETAKSPEALFVMTLNGDEKKKGRISATLNTKEGYSYLYIKG
SQPEDSATYLCALYNNNDMRFGAGTRLTVKPNiqnpdpavyqlrdskssdksvcl
ftdfdsqtnvsqskdsdvyitdktvldmrsmdfksnsavawsnksdfacanafnn
siipedtffpssdvpcdvklveksfetdtnlnfqnllvivlrilllkvagfnllm
tlrlws
R11P3D3-KE TCR MGTM codon optimized sequence
(used in working examples herein as
“Comparator Affinity Enhanced” and “Comparator AE TCR”)
Alpha chain:
TRAV24/TRAJ43/MGTM modified TRAC
Alpha chain DNA sequence
ATGGAGAAGAATCCTTTGGCAGCCCCATTACTAATCCTCTGGTTTCATCTTGACT
GCGTGAGCAGCATACTGAACGTGGAACAAAGTCCTCAGTCACTGCATGTTCAGGA
GGGAGACAGCACCAATTTCACCTGCAGCTTCCCTTCCAGCAATTTTTATGCCTTA
CACTGGTACAGAaaGGAAACTGCAAAAAGCCCCGAGGCCTTGTTTGTAATGACTT
TAAATGGGGATGAAAAGAAGAAAGGACGAATAAGTGCCACTCTTAATACCAAGGA
GGGTTACAGCTATTTGTACATCAAAGGATCCCAGCCTGAAGACTCAGCCACATAC
CTCTGTGCCCTGTACAATAACAATGACATGCGCTTTGGAGCAGGGACCAGACTGA
CAGTAAAACCAAacatccagaaccccgaccccgccgtgtaccagctgagggactc
caagtccagcgacaagagcgtgtgtctgtttacggacttcgacagccagaccaac
gtgagtcaaagcaaggacagcgacgtctacataacggataagaccgtgctggaca
tgcggagcatggacttcaagagcaacagcgccgtggcctggtccaacaagagcga
cttcgcctgcgccaacgccttcaacaacagcatcatccccgaggacaccttcttc
cccagcagcgacgtgccctgcgacgtgaaactggtggagaagtccttcgagacag
acaccaatctgaactttcagaacctgctggtgatcgtgctgcggattctgctgct
gaaagtggccggcttcaatctgctgatgaccctgcggctgtggagc
Alpha chain protein sequence
MEKNPLAAPLLILWFHLDCVSSILNVEQSPQSLHVQEGDSTNFTCSFPSSNFYAL
HWYRKETAKSPEALFVMTLNGDEKKKGRISATLNTKEGYSYLYIKGSQPEDSATY
LCALYNNNDMRFGAGTRLTVKPNiqnpdpavyqlrdskssdksvclftdfdsqtn
vsqskdsdvyitdktvldmrsmdfksnsavawsnksdfacanafnnsiipedtff
pssdvpcdvklveksfetdtnlnfqnllvivlrilllkvagfnllmtlrlws
Beta chain:
TRBV12-3/TRBJ2-3/MGTM modified TRBC
Beta chain DNA sequence
ATGGACTCCTGGACCTTCTGCTGTGTGTCCCTTTGCATCCTGGTAGCGAAGCATA
CAGATGCTGGAGTTATCCAGTCACCCCGCCATGAGGTGACAGAGATGGGACAAGA
AGTGACTCTGAGATGTAAACCAATTTCAGGCCACAACTCCCTTTTCTGGTACAGA
gAGACCATGATGCGGGGACTGGAGTTGCTCATTTACTTTAACAACAACGTTCCGA
TAGATGATTCAGGGATGCCCGAGGATCGATTCTCAGCTAAGATGCCTAATGCATC
ATTCTCCACTCTGAAGATCCAGCCCTCAGAACCCAGGGACTCAGCTGTGTACTTC
TGTGCCAGCAGTCCCGGCAGCACAGATACGCAGTATTTTGGCCCAGGCACCCGGC
TGACAGTGCTCGaagatctgaacaaggtgttccctccagaggtggccgtgttcga
gccttctaaggccgagatcgcccacacacaaaaagccaccctcgtgtgcctggcc
accggctttttccccgaccacgtggaactgtcttggtgggtcaacggcaaagagg
tgcactccggcgtgtcaacggatccccagcctctgaaagaacagcctgccctgaa
cgacagccggtactgcctgagctccagactgagagtgtccgccaccttctggcag
aacccccggaaccacttcagatgccaggtgcagttttacggcctgagcgagaacg
acgagtggacccaggacagagccaagcccgtgacacaaatcgtgtctgccgaagc
ctggggaagagccgattgcggcatcaccagcgcctcctatcaccagggcgtgctg
agcgccacaatcctgtacgaaatcctgctgggcaaggccaccctgtacgccgtgc
tggtgtctgctctggtgctgatggccatggtcaagcggaaggactttggcagcgg
cagagccaaaaggtccgggagcggt
Beta chain protein sequence
MDSWTFCCVSLCILVAKHTDAGVIQSPRHEVTEMGQEVTLRCKPISGHNSLFWYR
eTMMRGLELLIYFNNNVPIDDSGMPEDRESAKMPNASFSTLKIQPSEPRDSAVYF
CASSPGSTDTQYFGPGTRLTVLEdlnkvfppevavfepskaeiahtqkatlvcla
tgffpdhvelswwvngkevhsgvstdpqplkeqpalndsryclssrlrvsatfwq
nprnhfreqvqfyglsendewtqdrakpvtqivsaeawgradcgitsasyhqgvl
satilyeillgkatlyavlvsalvlmamvkrkdfgsgrakrsgsg
Complete Beta and Alpha ORF DNA Sequence
(The underlined italic region in the “Furin-
P2A” site encodes a sequence allowing for expression
of two polypeptide chains in a single
cassette)
ATGGACTCCTGGACCTTCTGCTGTGTGTCCCTTTGCATCCTGGTAGCGAAGCATA
CAGATGCTGGAGTTATCCAGTCACCCCGCCATGAGGTGACAGAGATGGGACAAGA
AGTGACTCTGAGATGTAAACCAATTTCAGGCCACAACTCCCTTTTCTGGTACAGA
gAGACCATGATGCGGGGACTGGAGTTGCTCATTTACTTTAACAACAACGTTCCGA
TAGATGATTCAGGGATGCCCGAGGATCGATTCTCAGCTAAGATGCCTAATGCATC
ATTCTCCACTCTGAAGATCCAGCCCTCAGAACCCAGGGACTCAGCTGTGTACTTC
TGTGCCAGCAGTCCCGGCAGCACAGATACGCAGTATTTTGGCCCAGGCACCCGGC
TGACAGTGCTCGaagatctgaacaaggtgttccctccagaggtggccgtgttcga
gccttctaaggccgagatcgcccacacacaaaaagccaccctcgtgtgcctggcc
accggctttttccccgaccacgtggaactgtcttgggggtcaacggcaaagaggt
gcactccggcgtgtcaacggatccccagcctctgaaagaacagcctgccctgaac
gacagccggtactgcctgagctccagactgagagtgtccgccaccttctggcaga
acccccggaaccacttcagatgccaggtgcagttttacggcctgagcgagaacga
cgagtggacccaggacagagccaagcccgtgacacaaatcgtgtctgccgaagcc
tggggaagagccgattgcggcatcaccagcgcctcctatcaccagggcgtgctga
gcgccacaatcctgtacgaaatcctgctgggcaaggccaccctgtacgccgtgct
ggtgtctgctctggtgctgatggccatggtcaagcggaaggactttggcagcggc
agagccaaaaggtccgggagcgctGCGACAAACTTTAGCCTGTTGAAACAAGCCG
GCGACGTTGAAGAGAACCCCGGACCTATGGAGAAGAATCCTTTGGCAGCCCCATT
ACTAATCCTCTGGTTTCATCTTGACTGCGTGAGCAGCATACTGAACGTGGAACAA
AGTCCTCAGTCACTGCATGTTCAGGAGGGAGACAGCACCAATTTCACCTGCAGCT
TCCCTTCCAGCAATTTTTATGCCTTACACTGGTACAGAaaGGAAACTGCAAAAAG
CCCCGAGGCCTTGTTTGTAATGACTTTAAATGGGGATGAAAAGAAGAAAGGACGA
ATAAGTGCCACTCTTAATACCAAGGAGGGTTACAGCTATTTGTACATCAAAGGAT
CCCAGCCTGAAGACTCAGCCACATACCTCTGTGCCCTGTACAATAACAATGACAT
GCGCTTTGGAGCAGGGACCAGACTGACAGTAAAACCAAacatccagaaccccgac
cccgccgtgtaccagctgagggactccaagtccagcgacaagagcgtgtgtctgt
ttacggacttcgacagccagaccaacgtgagtcaaagcaaggacagcgacgtcta
cataacggataagaccgtgctggacatgcggagcatggacttcaagagcaacagc
gccgtggcctggtccaacaagagcgacttcgcctgcgccaacgccttcaacaaca
gcatcatccccgaggacaccttcttccccagcagcgacgtgccctgcgacgtgaa
actggtggagaagtccttcgagacagacaccaatctgaactttcagaacctgctg
gtgatcgtgctgcggattctgctgctgaaagtggccggcttcaatctgctgatga
ccctgcggctgtggagc
Complete Beta and Alpha ORF Protein Sequence
(The underlined italic region in the “Furin-
P2A” site allows expression of two polypeptide chains
in a single cassette)
MDSWTFCCVSLCILVAKHTDAGVIQSPRHEVTEMGQEVTLRCKPISGHNSLFWYR
eTMMRGLELLIYFNNNVPIDDSGMPEDRESAKMPNASFSTLKIQPSEPRDSAVYF
CASSPGSTDTQYFGPGTRLTVLEdlnkvfppevavfepskaeiahtqkatlvcla
tgffpdhvelswwvngkevhsgvstdpqplkeqpalndsryclssrlrvsatfwq
nprnhfreqvqfyglsendewtqdrakpvtqivsaeawgradcgitsasyhqgvl
satilyeillgkatlyavlvsalvlmamvkrkdfgsgrakrsgsgATNFSLLKQA
GDVEENPGPMEKNPLAAPLLILWFHLDCVSSILNVEQSPQSLHVQEGDSTNFTCS
FPSSNFYALHWYRKETAKSPEALFVMTLNGDEKKKGRISATLNTKEGYSYLYIKG
SQPEDSATYLCALYNNNDMRFGAGTRLTVKPNiqnpdpavyqlrdskssdksvcl
ftdfdsqtnvsqskdsdvyitdktvldmrsmdfksnsavawsnksdfacanafnn
siipedtffpssdvpcdvklveksfetdtnlnfqnllvivlrilllkvagfnllm
tlrlws

| | | | |
|---|---|---|---|
| TCR | Chain | Region | Sequence |

Parental Original Comparator TCR Sequences (TCR R11P3D3)

| | | | |
|---|---|---|---|
| R11P3D3 | alpha | CDR1 | SSNFYA |
| R11P3D3 | alpha | CDR2 | MTL |
| R11P3D3 | alpha | CDR3 | CALYNNNDMRF |
| R11P3D3 | alpha | variable | MEKNPLAAPLLILWFHLDCVSSILNVEQSPQSLHVQEGD |
| | | domain | STNFTCSFPSSNFYALHWYRWETAKSPEA |
| | | | LFVMTLNGDEKKKGRISATLNTKEGYSYLYIKGSQPEDS |
| | | | ATYLCALYNNNDMRFGAGTRLTVKP |
| RIIP3D3 | alpha | constant | NIQNPDPAVYQLRDSKSSDKSVCLFTDFDSQTNVSQSKD |
| | | domain | SDVYITDKTVLDMRSMDFKSNSAVAWSN |
| | | | KSDFACANAFNNSIIPEDTFFPSPESSCDVKLVEKSPET |
| | | | DTNLNFQNLSVIGFRILLLKVAGFNLLMTLRLWSS |
| R11P3D3 | alpha | full- | MEKNPLAAPLLILWFHLDCVSSILNVEQSPQSLHVQEGD |
| | | length | STNFTCSFPSSNFYALHWYRWETAKSPEA |
| | | | LFVMTLNGDEKKKGRISATLNTKEGYSYLYIKGSQPEDS |
| | | | ATYLCALYNNNDMRFGAGTRLTVKPNIQNP |
| | | | DPAVYQLRDSKSSDKSVCLFTDFDSQTNVSQSKDSDVYI |
| | | | TDKTVLDMRSMDFKSNSAVAWSNKSDFA |
| | | | CANAFNNSIPEDTFFPSPESSCDVKLVEKSFETDTNLNF |
| | | | QNLSVIGFRILLLKVAGFNLLMTLRLWSS |
| R11P3D3 | beta | CDR1 | SGHNS |
| R11P3D3 | beta | CDR2 | FNNNVP |
| R11P3D3 | beta | CDR3 | CASSPGSTDTQYF |
| R11P3D3 | beta | variable | MDSWTFCCVSLCILVAKHTDAGVIQSPRHEVTEMGQEV |
| | | domain | TLRCKPISGHNSLFWYRQTMMRGLELLIYF |
| | | | NNNVPIDDSGMPEDRESAKMPNASFSTLKIQPSEPRDS |
| | | | AVYFCASSPGSTDTQYFGPGTRLTVL |
| R11P3D3 | beta | constant | EDLKNVFPPEVAVFEPSEAEISHTQKATLVCLATGFYP |
| | | domain | DHVELSWWVNGKEVHSGVSTDPQPLKEQP |
| | | | ALNDSRYCLSSRLRVSATFWQNPRNHFRCQVQFYGLSE |
| | | | NDEWTQDRAKPVTQIVSAEAWGRADCQF |
| | | | TSESYQQGVLSATILYEILLGKATLYAVLVSALVLMAM |
| | | | VKRKDSRG |
| R11P3D3 | beta | full- | MDSWTFCCVSLCILVAKHTDAGVIQSPRHEVTEMGQEV |
| | | length | TLRCKPISGHNSLFWYRQTMMRGLELLIYF |
| | | | NNNVPIDDSGMPEDRPSAKMPNASFSTLKIQPSEPRDS |
| | | | AVYFCASSPGSTDTQYFGPGTRLTVLEDLK |
| | | | NVFPPEVAVFEPSEAEISHTQKATLVCLATGFYPDHVE |
| | | | LSWWVNQKEVHSGVSTDPQPLKEQPALND |
| | | | SRYCLSSRLRVSATFWQNPRNHFRCQVQFYGLSENDEW |
| | | | TQDRAKPVTQIVSAEAWGRADCGFTSES |
| | | | YQQGVLSATILYEILLGKATLYAVLVSALVLMAMVKRK |
| | | | DSRG |

Parental Original Comparator Affinity Enhanced
TCR Sequences (TCR R11P3D3_KE)

| | | | |
|---|---|---|---|
| R11P3D3_ | alpha | CDR1 | SSNFYA |
| KE | | | |
| R11P3D3_ | alpha | CDR2 | MTL |
| KE | | | |
| RIIP3D3_ | alpha | CDR3 | CALYNNNDMRF |
| KE | | | |
| R11P3D3_ | alpha | variable | MEKNPLAAPLLILWFHLDCVSSILNVEQSPQSLHVQEQD |
| KE | | domain | STNFTCSFPSSNFYALHWYRKETAKSPEAL |
| | | | FVMTLNGDEKKKGRISATLNTKEGYSYLYIKGSQPEDSA |
| | | | TYLCALYNNNDMRFGAGTRLTVKP |
| R11P3D3_ | alpha | constant | NIQNPDPAVYQLRDSKSSDKSVCLFTDFDSQTNVSQSKD |
| KE | | domain | SDVYITDKTVLDMRSMDFKSNSAVAWSN |
| | | | KSDFACANAFNNSUPEDTFFPSPESSCDVKLVEKSFETD |
| | | | INLNFQNLSVIGFRILLLKVAGENLLMTLRLWSS |
| R11P3D3_ | alpha | full- | MEKNPLAAPLLILWFHLDCVSSILNVEQSPQSLHVQEGD |
| KE | | length | STNFTCSFPSSNFYALHWYRKETAKSPEAL |
| | | | FVMTLNGDEKKKGRISATENTKEGYSYLYIKGSQPEDSA |
| | | | TYLCALYNNNDMRFGAGTRLTVKPNIQNP |
| | | | DPAVYQLRDSKSSDKSVCLFTDFDSQTNVSQSKDSDVYI |
| | | | TDKTVLDMRSMDFKSNSAVAWSNKSDFA |
| | | | CANAFNNSHIPEDTFFPSPESSCDVKLVEKSFETDTNLN |
| | | | FQNLSVIGFRILLLKVAGFNLLMTLRLWSS |
| RIIP3D3_ | beta | CDR1 | SGHNS |
| KE | | | |
| R11P3D3_ | beta | CDR2 | FNNNVP |
| KE | | | |
| R11P3D3_ | beta | CDR3 | CASSPGSTDTQYF |
| KE | | | |
| R11P3D3_ | beta | variable | MDSWTFCCVSLCILVAKHTDAGVIQSPRHEVTEMGQEVTL |
| KE | | domain | RCKPISGHNSLFWYRETMMRGLELLIYPF |
| | | | NNNVPIDDSGMPEDRFSAKMPNASFSTLKIQPSEPRDSAV |
| | | | YFCASSPGSTDTQYFGPGTRLTVL |
| R11P3D3_ | beta | constant | EDLKNVFPPEVAVFEPSEAEISHTQKATLVCLATGFYPDH |
| KE | | domain | VELSWWVNGKEVHISGVSTDPQPLKEQP |
| | | | ALNDSRYCLSSRLRVSATFWQNPRNHFRCQVQFYGLSEND |
| | | | EWTQDRAKPVTQIVSAEAWGRADCGF |
| | | | TSESYQQGVLSATILYEILLGKATLYAVLVSALVLMAMVK |
| | | | RKDSRG |
| R11P3D3_ | beta | full- | MDSWTFCCVSLCILVAKHTDAGVIQSPRHEVTEMGQEVTL |
| KE | | length | RCKPISGHNSLFWYRETMMRGLELLIYF |
| | | | NNNVPIDDSGMPEDRFSAKMPNASFSTLKIQPSEPRDSAV |
| | | | YFCASSPGSTDTQYFGPGTRLTVLEDLK |
| | | | NVFPPEVAVFEPSEAFISHTQKATLVCLATGFYPDHVELS |
| | | | WWVNGKEVHSGVSTDPQPLKEQPALND |
| | | | SRYCLSSRLRVSATFWQNPRNHFRCQVQFYGLSENDEWTQ |
| | | | DRAKPVTQIVSAEAWGRADCGFTSES |
| | | | YQQGVLSATILYEILLGKATLYAVLVSALVLMAMVKRKDS |
| | | | RG |

Representative Vector (the TCR-encoding protein of which
can be interchanged with any TCR sequence of interest):
pNVVD134_TSC-203-A02_TCR-366_MSCV-TCR-366-
CD8-EF1α-dnTGFbRII-DHFR
GCTAGCTGGCTTGTTGTCCACAACCATTAAACCTTAAAAGCTTTAAAAGCCTTAT
ATATTCTTTTTTTTCTTATAAAACTTAAAACCTTAGAGGCTATTTAAGTTGCTGA
TTTATATTAATTTTATTGTTCAAACATGAGAGCTTAGTACGTGAAACATGAGAGC
TTAGTACATTAGCCATGAGAGCTTAGTACATTAGCCATGAGGGTTTAGTTCATTA
AACATGAGAGCTTAGTACATTAAACATGAGAGCTTAGTACATACTATCAACAGGT
TGAACTGCTGATCTGTACAGTAGAATTGGTAAAGAGAGTTGTGTAAAATATTGAG
TTCGCACATCTTGTTGTCTGATTATTGATTTTTGGCGAAACCATTIGATCATATG
ACAAGATGTGTATCTACCTTAACTTAATGATTTTGATAAAAATCATTAGGTACCA
ATTACATTGCTTGCAATTAACCCTTTAACGGTTATAAGGATCTAGATGAGATAGA
AAGATTTGGTTTTCGGATTTGTGTTACATAAGATGCCTAAAATAAAAATTGAGAT
TCAATTTTTTTTAAACTTTTTTTTAATTGGTGGTAAGAATATTCCCTCTACCTGT
TTGAGAGTAATGAAATTGTAGTATGATTTTTCAACAAACTAAAAAAACAACATAA
ATCTCACATAATAACTTTATTTCAATCACACAATTGAATACCAATAGGTTGACAG
TACTTACCAGCCTGCAGGTGAAAGACCCCACCTGTAGGTTTGGCAAGTTAGCTTA
AGTAACGCCATTTTGCAAGGCATGGAAAATACATAACTGAGAATAGAGAAGTTCA
GATCAAGGTTAGGAACAGAGAGACAGCAGAATATGGGCCAAACAGGATATCTGTG
GTAAGCAGTTCCTGCCCCGGCTCAGGGCCAAGAACAGATGGTCCCCAGATGCGGT
CCCGCCCTCAGCAGTTTCTAGCGAACCATCAGATGTTTCCAGGGTGCCCCAAGGA
CCTGAAATGACCCTGTGCCTTATTTGAACTAACCAATCAGTTTGCTTCTTGCTTC
TGTTTGTGTGCTTCTGCTCCCTGAGCTCAATAAAAGAGCCCACAACCCCTCACTT
GGTGGGCCAGTCCTCTGATAGACTGTGTCCCCTGGATACCCGTACGGTACCGCTA
GCGCCACCATGCTGAGCCCCGACCTGCCTGACAGCGCTTGGAATACCAGACTCCT
GTGCAGAGTGATGCTGTGCCTGCTTGGAGCTGGAAGTGTGGCTGCTGGTGTCATT
CAGTCCCCAAGGCACCTGATCAAAGAGAAGAGAGAGACAGCCACTCTGAAGTGCT
ACCCCATTCCTAGACACGACACGGTCTATTGGTATCAGCAAGGACCTGGACAGGA
CCCTCAGTTCCTGATCAGCTTCTACGAGAAGATGCAGAGCGACAAGGGCAGCATC
CCCGACAGATTTTCTGCCCAGCAGTTCAGCGACTACCACAGCGAGCTGAACATGA
GCAGCCTGGAACTGGGCGATAGCGCCCTGTACTTCTGTGCCTCTTCTTTCGCACG
CCTGGAAGGTCGCGATAATGAACAATTTTTTGGGCCAGGGACACGGCTCACCGTG
CTAGAAGATCTGAACAAGGTGTTCCCTCCAGAGGTGGCCGTGTTCGAGCCTTCTA
AGGCCGAGATCGCCCACACACAAAAAGCCACCCTCGTGTGCCTGGCCACCGGCTT
TTTCCCCGACCACGTGGAACTGTCTTGGTGGGTCAACGGCAAAGAGGTGCACTCC
GGCGTGTCAACGGATCCCCAGCCTCTGAAAGAACAGCCTGCCCTGAACGACAGCC
GGTACTGCCTGAGCTCCAGACTGAGAGTGTCCGCCACCTTCTGGCAGAACCCCCG
GAACCACTTCAGATGCCAGGTGCAGTTTTACGGCCTGAGCGAGAACGACGAGTGG
ACCCAGGACAGAGCCAAGCCCGTGACACAAATCGTGTCTGCCGAAGCCTGGGGAA
GAGCCGATTGCGGCATCACCAGCGCCTCCTATCACCAGGGCGTGCTGAGCGCCAC
AATCCTGTACGAAATCCTGCTGGGCAAGGCCACCCTGTACGCCGTGCTGGTGTCT
GCTCTGGTGCTGATGGCCATGGTCAAGCGGAAGGACTTTGGCAGCGGCAGAGCCA
AAAGGTCCGGGAGCGGTGCGACAAACTTTAGCCTGTTGAAACAAGCCGGCGACGT
TGAAGAGAACCCCGGACCTATGGCCTGTCCTGGCTTCCTGTGGGCCCTTGTGATC
AGCACTTGCCTGGAATTCAGCATGGCTCAGACAGTCACCCAGTCTCAGCCCGAAA
TGAGCGTCCAAGAGGCTGAAACCGTGACTCTGTCTTGTACCTACGACACCTCCGA
GAGCGATTACTACCTCTTTTGGTATAAGCAACCGCCGTCCAGGCAAATGATCCTC
GTGATCCGGCAAGAAGCTTACAAACAGCAGAATGCTACCGAAAACCGGTTCTCCG
TCAATTTTCAGAAAGCCGCTAAGAGCTTTAGCCTGAAAATCTCCGACTCTCAGCT
CGGCGACGCTGCTATGTATTTCTGTGCCTACCGCAAAACTTCTTACGATAAAGTC
ATTTTTGGGCCAGGGACAAGCTTATCAGTCATTCCAAACATCCAGAACCCCGACC
CCGCCGTGTACCAGCTGAGGGACTCCAAGTCCAGCGACAAGAGCGTGTGTCTGTT
TACGGACTTCGACAGCCAGACCAACGTGAGTCAAAGCAAGGACAGCGACGTCTAC
ATAACGGATAAGACCGTGCTGGACATGCGGAGCATGGACTTCAAGAGCAACAGCG
CCGTGGCCTGGTCCAACAAGAGCGACTTCGCCTGCGCCAACGCCTTCAACAACAG
CATCATCCCCGAGGACACCTTCTTCCCCAGCAGCGACGTGCCCTGCGACGTGAAA
CTGGTGGAGAAGTCCTTCGAGACAGACACCAATCTGAACTTTCAGAACCTGCTGG
TGATCGTGCTGCGGATTCTGCTGCTGAAAGTGGCCGGCTTCAATCTGCTGATGAC
CCTGCGGCTGTGGAGCAGCAGGGCTAAGAGGTCCGGCAGCGGAGCCACCAATTTT
TCCCTGCTGAAACAGGCTGGTGACGTGGAAGAAAACCCTGGCCCCATGGCGCTGC
CCGTCACCGCGCTGCTGCTGCCCCTGGCGCTGCTGTTACACGCCGCTCGGCCAGA
GCTTCCCACCCAGGGCACATTCTCCAACGTGTCCACCAATGTGTCGGGAGGCGGC
GGATCGTCCCAGTTCAGAGTGTCCCCTCTGGACCGCACCTGGAACCTGGGCGAGA
CCGTGGAGCTGAAATGTCAGGTCCTGCTGAGCAACCCGACCTCCGGGTGCAGTTG
GCTGTTCCAGCCGCGTGGTGCTGCCGCAAGCCCTACGTTCCTGCTTTACCTGAGC
CAGAACAAGCCCAAGGCGGCCGAGGGCCTGGACACCCAGAGATTCTCCGGCAAGC
GCCTGGGGGACACATTCGTGCTTACTTTGAGCGATTTCCGCAGAGAGAACGAGGG
CTACTATTTCTGTTCGGCGCTGAGCAATTCCATCATGTATTTCAGCCACTTTGTG
CCAGTGTTCCTGCCTGCCAAGCCTACCACAACACCAGCTCCCCGTCCCCCGACTC
CGGCGCCTACCATCGCGAGTCAACCGTTGAGCCTGAGGCCTGAGGCTTGTCGGCC
CGCTGCGGGGGGTGCCGTCCACACCAGGGGCCTCGACTTTGCGTGCGACATCTAT
ATTTGGGCGCCTCTGGCGGGTACCTGCGGGGTGCTGCTGCTGTCATTGGTGATTA
CCCTGTACTGCAATCACCGCAACCGCCGGCGGGTCTGTAAGTGCCCACGGCCTGT
GGTCAAGTCCGGTGACAAACCGTCGCTCTCGGCTCGCTACGTGCGCGCTAAGCGC
AGCGGTTCCGGGGCCACCAACTTTTCATTGCTGAAGCAGGCCGGTGATGTGGAGG
AGAATCCAGGGCCCATGCGCCCCAGGCTTTGGCTCCTTCTTGCTGCTCAGCTCAC
TGTCTTGCATGGCAACTCCGTTCTGCAGCAGACTCCCGCCTACATCAAGGTGCAG
ACGAACAAGATGGTGATGCTGTCATGCGAGGCCAAGATCTCTCTTTCAAATATGA
GAATTTATTGGCTACGACAGCGCCAGGCCCCCTCCAGCGACAGCCACCACGAGTT
CCTGGCGCTTTGGGATTCTGCTAAAGGCACCATCCATGGAGAGGAGGTGGAACAG
GAGAAGATAGCTGTCTTCCGCGACGCATCCCGCTTCATCCTGAACCTGACCAGCG
TGAAGCCGGAGGACAGCGGCATCTACTTCTGTATGATCGTTGGCTCCCCCGAGCT
GACCTTCGGCAAAGGCACCCAGCTGTCCGTGGTGGACTTCCTGCCCACCACAGCC
CAGCCAACCAAGAAATCCACCCTCAAGAAGCGCGTGTGCCGACTGCCCCGCCCTG
AAACCCAGAAGGGCCCTCTGTGCTCCCCCATCACCCTTGGACTGCTGGTGGCGGG
AGTCCTGGTGCTGCTCGTATCTCTGGGTGTCGCCATCCACCTGTGCTGCCGCCGC
CGCCGCGCCCGCCTGAGGTTTATGAAACAGTTTTACAAGTGATAAATCGATGGAA
GGGTGGCATCCCTGTGACCCCTCCCCAGTGCCTCTCCTGGCCCTGGAAGTTGCCA
CTCCAGTGCCCACCAGCCTTGTCCTAATAAAATTAAGTTGCATCATTTTGTCTGA
CTAGGTGTCCTTCTATAATATTATGGGGTGGAGGGGGGTGGTATGGAGCAAGGGG
CAAGTTGGGAAGACAACCTGTAGGGCCTGCGGGGTCTATTGGGAACCAAGCTGGA
GTGCAGTGGCACAATCTTGGCTCACTGCAATCTCCGCCTCCTGGGTTCAAGCGAT
TCTCCTGCCTCAGCCTCCCGAGTTGTTGGGATTCCAGGCATGCATGACCAGGCTC
AGCTAATTTTTGTTTTTTTGGTAGAGACGGGGTTTCACCATATTGGCCAGGCTGG
TCTCCAACTCCTAATCTCAGGTGATCTACCCACCTTGGCCTCCCAAATTGCTGGG
ATTACAGGCGTGAACCACTGCTCCCTTCCCTGTCCTTCTGATTACTAGTGGCTCC
GGTGCCCGTCAGTGGGCAGAGCGCACATCGCCCACAGTCCCCGAGAAGTIGTGGG
GAGGGGTCGGCAATTGAACCGGTGCCTAGAGAAGGTGGCGCGGGGTAAACTGGGA
AAGTGATGTCGTGTACTGGCTCCGCCTTTTTCCCGAGGGTGGGGGAGAACCGTAT
ATAAGTGCAGTAGTCGCCGTGAACGTTCTTTTTCGCAACGGGTTTGCCGCCAGAA
CACAGGTAAGTGCCGTGTGTGGTTCCCGCGGGCCTGGCCTCTTTACGGGTTATGG
CCCTTGCGTGCCTTGAATTACTTCCACCTGGCTGCAGTACGTGATTCTTGATCCC
GAGCTTCGGGTTGGAAGTGGGTGGGAGAGTTCGAGGCCTTGCGCTTAAGGAGCCC
CTTCGCCTCGTGCTTGAGTTGAGGCCTGGCCTGGGCGCTGGGGCCGCCGCGTGCG
AATCTGGTGGCACCTTCGCGCCTGTCTCGCTGCTTTCGATAAGTCTCTAGCCATT
TAAAATTTTTGATGACCTGCTGCGACGCTTTTTTTCTGGCAAGATAGTCTTGTAA
ATGCGGGCCAAGATCTGCACACTGGTATTTCGGTTTTTGGGGCCGCGGGGGCGAC
GGGGCCCGTGCGTCCCAGCGCACATGTTCGGCGAGGCGGGGCCTGCGAGCGCGGC
CACCGAGAATCGGACGGGGGTAGTCTCAAGCTGGCCGGCCTGCTCTGGTGCCTGG
CCTCGCGCCGCCGTGTATCGCCCCGCCCTGGGCGGCAAGGCTGGCCCGGTCGGCA
CCAGTTGCGTGAGCGGAAAGATGGCCGCTTCCCGGCCCTGCTGCAGGGAGCTCAA
AATGGAGGACGCGGCGCTCGGGAGAGCGGGGGGTGAGTCACCCACACAAAGGAAA
AGGGCCTTTCCGTCCTCAGCCGTCGCTTCATGTGACTCCACGGAGTACCGGGCGC
CGTCCAGGCACCTCGATTAGTTCTCGAGCTTTTGGAGTACGTCGTCTTTAGGTTG
GGGGGAGGGGTTTTATGCGATGGAGTTTCCCCACACTGAGTGGGTGGAGACTGAA
GTTAGGCCAGCTTGGCACTTGATGTAATTCTCCTTGGAATTTGCCCTTTTTGAGT
TTGGATCTTGGTTCATTCTCAAGCCTCAGACAGTGGTTCAAAGTTTTTTTCTTCC
ATTTCAGGTGTCGTGAACTAGTCCAGTGTGGTGGAATTCTGCAGATATCACGGCT
AGCGCCACCATGGGTCGGGGGCTGCTCAGGGGCCTGTGGCCGCTGCACATCGTCC
TGTGGACGCGTATCGCCAGCACGATCCCACCGCACGTTCAGAAGTCGGTGAATAA
CGACATGATAGTCACTGACAACAACGGTGCAGTCAAGTTTCCACAACTGTGTAAA
TTTTGTGATGTGAGATTTTCCACCTGTGACAACCAGAAATCCTGCATGAGCAACT
GCAGCATCACCTCCATCTGTGAGAAGCCACAGGAAGTCTGTGTGGCTGTATGGAG
AAAGAATGACGAGAACATAACACTAGAGACAGTTTGCCATGACCCCAAGCTCCCC
TACCATGACTTTATTCTGGAAGATGCTGCTTCTCCAAAGTGCATTATGAAGGAGA
AGAAAAAGCCTGGTGAGACTTTCTTCATGTGTTCCTGTAGCTCTGATGAGTGCAA
TGACAACATCATCTTCTCAGAAGAATATAACACCAGCAATCCTGACTTGTTGCTA
GTCATATTTCAAGTGACAGGCATCAGCCTCCTGCCACCACTGGGAGTTGCCATAT
CTGTCATCATCATCTTCTACTGCTACCGCGTGAACCGGCAGCAGAAGGCTAGTGG
TTCAGGCGCAACGAATTTCTCTTTGCTGAAGCAGGCTGGGGATGTCGAAGAAAAT
CCGGGTCCAATGGTGGGCTCGCTCAACTGCATCGTAGCAGTCTCCCAGAATATGG
GCATCGGGAAGAACGGTGATTTCCCGTGGCCCCCACTTCGCAACGAGAGCCGTTA
TTTCCAAAGAATGACTACAACCTCCTCCGTGGAGGGTAAGCAGAACCTGGTCATC
ATGGGGAAGAAGACCTGGTTCTCTATCCCTGAAAAAAACCGCCCCCTGAAGGGCC
GCATCAACCTGGTGCTGAGCAGGGAACTCAAGGAGCCTCCTCAGGGCGCGCATTT
TCTGAGCCGCTCATTGGATGACGCTCTCAAACTGACCGAACAGCCGGAGCTAGCC
AACAAGGTGGACATGGTGTGGATCGTCGGAGGCTCCTCCGTGTACAAGGAGGCCA
TGAATCACCCCGGCCACTTGAAGCTGTTCGTCACCCGGATCATGCAGGACTTCGA
GTCGGACACGTTCTTTCCAGAGATTGACCTGGAGAAGTACAAGCTGCTGCCCGAG
TACCCGGGAGTTCTTAGTGATGTGCAGGAGGAGAAAGGCATCAAGTACAAATTTG
AGGTGTACGAGAAGAACGACTAACGGTCCGTCCTGACCAATGCTGGAGTTCTTCG
CCCACCCCAACTTGTTTATTGCAGCTTATAATGGTTACAAATAAAGCAATAGCAT
CACAAATTTCACAAATAAAGCATTTTTTTCACTGCATTCTAGTTGTGGTTTGTCC
AAACTCATCAATGTATCTTATCATGTCTGTATACAGGTTACCTCAGTCTCCTAGG
TACGTCTTATATCTATGAAAAAACATTCAAAAGCACAACATCTAGAAGAACTTAC
CTTTTTTCACCACTCTATTGCAAAGATATGTACCGATTTCTCTCGAAGTACAAAA
AACCGCTAGTTTTCAAATTCACCTCAAGACTTTGAAAAAAAATTGAATCTGTCAA
TGTCAAATAAAATCAGAAACAAATGTCATAATGTTACGTTAATGTTGTCAGGTCG
AAAAATAAAATTGCAAATAGAAATTTTGTTCCTTTTTTATTGGTTTTTATTGGTG
GGAAAAATATTCCCTCTAACTGCAAAAGGGTTAATTATGTTAGAGGTAGAGTCGA
C
* For certain depicted vectors, MSCV promoter is in bold. Beta chain is annotated using bold and italic text. Alpha chain is annotated using bold and underlined text. CD34-enrichment tag (Q tag) is annotated using italic and underlined text. CD8-alpha is in italic. CD8-beta is underlined.
* Included in Tables 1-4 herein are peptide epitopes, as well as polypeptide molecules comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, 99.5%, or more identity across their full length with an amino acid sequence of any sequences listed in Tables 1-4, or a portion thereof. Such polypeptides may have a function of the full-length peptide or polypeptide as described further herein.
* Included in Tables 1-4 are RNA nucleic acid molecules (e.g., thymines replaced with uredines), nucleic acid molecules encoding orthologs of the encoded proteins, as well as DNA or RNA nucleic acid sequences comprising a nucleic acid sequence having at least 80%, 81%, 82%, 83%, 84%, 85%, 86%, 87%, 88%, 89%, 90%, 91%, 92%, 93%, 94%, 95%, 96%, 97%, 98%, 99%, 99.5%, or more identity across their full length with the nucleic acid sequence of any sequence listed in Tables 1-4, or a portion thereof. Such nucleic acid molecules can have a function of the full-length nucleic acid as described further herein.

In some embodiments, the binding proteins provided herein comprise a constant region that is chimeric, humanized, human, primate, or rodent (e.g., rat or mouse). For example, a human variable region may be chimerized with a murine constant region or a murine variable region may be humanized with a human constant region and/or human framework-regions. In some embodiments, the constant regions may be mutated to modify functionality (e.g., introduction of non-naturally occurring cysteine substitutions in opposing residue locations in TCR alpha and beta chains to provide disulfide bonds useful for increasing affinity between the TCR alpha and beta chains). Similarly, mutations may be made in the transmembrane domain of the constant region to modify functionality (e.g., increase hydrophobicity by introducing a non-naturally occurring substitution of a residue with a hydrophobic amino acid). In some embodiments, each CDR of the binding protein has up to five amino acid substitutions, insertions, deletions, or a combination thereof as compared to a reference CDR sequence. In some embodiments, mutations may be made to the constant region to increase cell surface expression.

In some embodiments, the binding proteins disclosed herein may be engineered protein scaffolds, an antibody or an antigen-binding fragment thereof, TCR-mimic antibodies, and the like. Such binding moieties may be designed and/or generated against peptides and/or MHC-peptide complexes described herein using routine immunological methods, such as immunizing a host, obtaining antibody-producing cells and/or antibodies thereof, and generating hybridomas useful for producing monoclonal antibodies (e.g., Watt et al. (2006) Nat. Biotechnol, 24:177-183; Gebauer and Skerra (2009) Curr. Opin. Chem Biol. 13:245-255; Skerra et al. (2008) FEBS J, 275:2677-2683; Nygren et al. (2008) FEBS J. 275:2668-2676; Dana et al. (2012) Exp. Rev. Mol. Med, 14:e6; Sergeva et al. (2011) Blood 117:4262-4272; PCT Publ. Nos. WO 2007/143104, PCT/US86/02269, and WO 86/01533; U.S. Pat. No. 4,816,567; Better et al. (1988) Science 240:1041-1043; Liu et al. (1987) Proc. Natl. Acad. Sci. U.S.A, 84:3439-3443; Liu et al. (1987) J. Immunol, 139:3521-3526; Sun et al. (1987) Proc. Natl. Acad. Sci, 84:214-218; Nishimura et al. (1987) Cancer Res, 47:999-1005; Wood et al. (1985) Nature 314:446-449; Shaw et al. (1988) J. Natl. Cancer Inst. 80:1553-1559); Morrison, S. L. (1985) Science 229:1202-1207; Oi et al. (1986) Biotechniques 4:214; U.S. Pat. No. 5,225,539; Jones et al. (1986) Nature 321:552-525; Verhoeyan et al. (1988) Science 239:1534; and Beidler et al. (1988) J. Immunol, 141:4053-4060. If desired, binding moieties may be isolated or purified using conventional procedures such as, for example, protein A-Sepharose, hydroxylapatite chromatography, gel electrophoresis, dialysis, affinity chromatography, ammonium sulfate or ethanol precipitation, acid extraction, anion or cation exchange chromatography, phosphocellulose chromatography, hydrophobic interaction chromatography, hydroxylapatite chromatography, lectin chromatography, and high performance liquid chromatography (HPLC) (e.g., Current Protocols in Immunology, or Current Protocols in Protein Science. John Wiley & Sons, NY, N.Y.).

The terms “antibody” and “antibodies” broadly encompass naturally-occurring forms of antibodies (e.g. IgG, IgA, IgM, IgE) and recombinant antibodies, such as single-chain antibodies, chimeric and humanized antibodies and multi-specific antibodies, as well as fragments and derivatives of all of the foregoing, which fragments and derivatives have at least an antigenic binding site. Antibody derivatives may comprise a protein or chemical moiety conjugated to an antibody.

In addition, intrabodies are well-known antigen-binding molecules having the characteristic of antibodies, but that are capable of being expressed within cells in order to bind and/or inhibit intracellular targets of interest (Chen et al. (1994) Human Gene Ther. 5:595-601). Methods are well-known in the art for adapting antibodies to target (e.g., inhibit) intracellular moieties, such as the use of single-chain antibodies (scFvs), modification of immunoglobulin VL domains for hyperstability, modification of antibodies to resist the reducing intracellular environment, generating fusion proteins that increase intracellular stability and/or modulate intracellular localization, and the like. Intracellular antibodies can also be introduced and expressed in one or more cells, tissues or organs of a multicellular organism, for example for prophylactic and/or therapeutic purposes (e.g., as a gene therapy) (see, at least PCT Publ. Nos. WO 08/020079, WO 94/02610. WO 95/22618, and WO 03/014960; U.S. Pat. No. 7,004,940; Cattaneo and Biocca (1997) Intracellular Antibodies: Development and Applications (Landes and Springer-Verlag publs.); Kontermann (2004) Methods 34:163-170; Cohen et al. (1998) Oncogene 17:2445-2456; Auf der Maur et al. (2001) FEBS Lett. 508:407-412; Shaki-Loewenstein et al. (2005) J. Immunol. Meth. 303:19-39).

The term “antibody” as used herein also includes an “antigen-binding portion” of an antibody (or simply “antibody portion”). The term “antigen-binding portion”, as used herein, refers to one or more fragments of an antibody that retain the ability to specifically and/or selectively bind to an antigen (e.g., a peptide and/or an MHC-peptide complex described herein). It has been shown that the antigen-binding function of an antibody can be performed by fragments of a full-length antibody. Examples of binding fragments encompassed within the term “antigen-binding portion” of an antibody include (i) a Fab fragment, a monovalent fragment consisting of the VL, VH, CL and CH1 domains; (ii) a F(ab′)₂ fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody. (v) a dAb fragment (Ward et al., (1989) Nature 341:544-546), which consists of a VH domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent polypeptides (known as single chain Fv (scFv); see e.g., Bird et al. (1988) Science 242:423-426; and Huston et al. (1988) Proc. Natl. Acad. Sci. USA 85:5879-5883; and Osbourn et al, 1998. Nature Biotechnology 16: 778). Such single chain antibodies are also intended to be encompassed within the term “antigen-binding portion” of an antibody. Any VH and VL sequences of specific scFv can be linked to human immunoglobulin constant region cDNA or genomic sequences, in order to generate expression vectors encoding complete IgG polypeptides or other isotypes. VH and VL can also be used in the generation of Fab, Fv or other fragments of immunoglobulins using either protein chemistry or recombinant DNA technology. Other forms of single chain antibodies, such as diabodies are also encompassed. Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites (see e.g., Holliger et al. (1993) Proc. Natl. Acad. Sci. U.S.A. 90:6444-6448; Poljak et al. (1994) Structure 2:1121-1123).

Still further, an antibody or antigen-binding portion thereof may be part of larger immunoadhesion polypeptides, formed by covalent or noncovalent association of the antibody or antibody portion with one or more other proteins or peptides. Examples of such immunoadhesion polypeptides include use of the streptavidin core region to make a tetrameric scFv polypeptide (Kipriyanov et al. (1995) Human Antibodies and Hybridomas 6:93-101) and use of a cysteine residue, protein subunit peptide and a C-terminal polyhistidine tag to make bivalent and biotinylated scFv polypeptides (Kipriyanov et al. (1994) Mol. Immunol, 31:1047-1058). Antibody portions, such as Fab and F(ab′)₂ fragments, can be prepared from whole antibodies using conventional techniques, such as papain or pepsin digestion, respectively, of whole antibodies. Moreover, antibodies, antibody portions and immunoadhesion polypeptides can be obtained using standard recombinant DNA techniques, as described herein.

Antibodies may be polyclonal or monoclonal: xenogeneic, allogeneic, or syngeneic; or modified forms thereof (e.g. humanized, chimeric, etc.). Antibodies may also be fully human. Preferably, antibodies of the invention bind specifically and/or selectively or substantially specifically and/or selectively to a peptide and/or an MHC-peptide complex described herein. The terms “monoclonal antibodies” and “monoclonal antibody composition”, as used herein, refer to a population of antibody polypeptides that contain only one species of an antigen binding site capable of immunoreacting with a particular epitope of an antigen, whereas the term “polyclonal antibodies” and “polyclonal antibody composition” refer to a population of antibody polypeptides that contain multiple species of antigen binding sites capable of interacting with a particular antigen. A monoclonal antibody composition typically displays a single binding affinity for a particular antigen with which it immunoreacts.

Similar to other binding moieties described herein, antibodies may also be “humanized,” which is intended to include antibodies made by a non-human cell having variable and constant regions which have been altered to more closely resemble antibodies that would be made by a human cell. For example, by altering the non-human antibody amino acid sequence to incorporate amino acids found in human germline immunoglobulin sequences. The humanized antibodies of the invention may include amino acid residues not encoded by human germline immunoglobulin sequences (e.g., mutations introduced by random or site-specific mutagenesis in vitro/ex vivo or by somatic mutation in vivo), for example in the CDRs. The term “humanized antibody”, as used herein, also includes antibodies in which CDR sequences derived from the germline of another mammalian species, have been grafted onto human framework sequences.

In some embodiments, the binding proteins disclosed herein may comprise a T cell receptor (TCR), an antigen-binding fragment of a TCR, or a chimeric antigen receptor (CAR). In some embodiments, the binding protein disclosed herein may comprise two polypeptide chains, each of which comprises a variable region comprising a CDR3 of a TCR alpha chain and a CDR3 of a TCR beta chain, or a CDR1, CDR2, and CDR3 of both a TCR alpha chain and a TCR beta chain. In some embodiments, a binding protein comprises a single chain TCR (scTCR), which comprises both the TCR V_{α} and TCR V_{β} domains, but only a single TCR constant domain (C_{α} or C_{β}). The term “chimeric antigen receptor” (CAR) refers to a fusion protein that is engineered to contain two or more naturally-occurring amino acid sequences linked together in a way that does not occur naturally or does not occur naturally in a host cell, which fusion protein can function as a receptor when present on a surface of a cell. CARs encompassed by the present invention may include an extracellular portion comprising an antigen-binding domain (i.e., obtained or derived from an immunoglobulin or immunoglobulin-like molecule, such as an antibody or TCR, or an antigen binding domain derived or obtained from a killer immunoreceptor from an NK cell) linked to a transmembrane domain and one or more intracellular signaling domains (optionally containing co-stimulatory domain(s)) (see, e.g., Sadelain et al. (2013) Cancer Discov, 3:388, Harris and Kranz (2016) Trends Pharmacol. Sci, 37:220, and Stone et al. (2014) Cancer Immunol. Immunother, 63:1163).

In some embodiments, 1) the TCR alpha chain CDR, TCR V_{α} domain, and/or TCR alpha chain is encoded by a TRAV, TRAJ, and/or TRAC gene or fragment thereof selected from the group of TRAV, TRAJ, and TRAC genes listed in Table 2, and/or 2) the TCR beta chain CDR, TCR V_{β} domain, and/or TCR beta chain is encoded by a TRBV. TRBJ, and/or TRBC gene or fragment thereof selected from the group of TRBV, TRBJ, and TRBC genes listed in Table 2, and/or 3) each CDR of the binding protein has up to five amino acid substitutions, insertions, deletions, or a combination thereof as compared to the cognate reference CDR sequence listed in Table 2.

In some embodiments, the binding proteins (e.g., the TCR, antigen-binding fragment of a TCR, or chimeric antigen receptor (CAR)) disclosed herein is chimeric (e.g., comprises amino acid residues or motifs from more than one donor or species), humanized (e.g., comprises residues from a non-human organism that are altered or substituted so as to reduce the risk of immunogenicity in a human), or human.

Methods for producing engineered binding proteins, such as TCRs, CARs, and antigen-binding fragments thereof, are well-known in the art (e.g., Bowerman et al. (2009) Mol. Immunol, 5:3000; U.S. Pat. Nos. 6,410,319; 7,446,191; U.S. Pat. Publ. No. 2010/065818; U.S. Pat. No. 8,822,647; PCT Publ. No. WO 2014/031687; U.S. Pat. No. 7,514,537; and Brentjens et al. (2007) Clin. Cancer Res, 73:5426).

In some embodiments, the binding protein described herein is a TCR, or antigen-binding fragment thereof, expressed on a cell surface, wherein the cell surface-expressed TCR is capable of more efficiently associating with a CD3 protein as compared to endogenous TCR. A binding protein encompassed by the present invention, such as a TCR, when expressed on the surface of a cell like a T cell, may also have higher surface expression on the cell as compared to an endogenous binding protein, such as an endogenous TCR. In some embodiments, provided herein is a CAR, wherein the binding domain of the CAR comprises an antigen-specific TCR binding domain (see, e.g., Walseng et al. (2017) Scientific Reports 7:10713).

Also provided are modified binding proteins (e.g., TCRs, antigen-binding fragments of TCRs, or CARs) that may be prepared according to well-known methods using a binding protein having one or more of the V_{α} and/or V_{β} sequences disclosed herein as starting material to engineer a modified binding protein that may have altered properties from the starting binding protein. A binding protein may be engineered by modifying one or more residues within one or both variable regions (i.e., V_{α} and/or V_{β}), for example within one or more CDR regions and/or within one or more framework regions. Additionally or alternatively, a binding protein may be engineered by modifying residues within the constant region(s).

Another type of variable region modification is to mutate amino acid residues within the V_{α} and/or V_{β} CDR1, CDR2 and/or CDR3 regions to thereby improve one or more binding properties (e.g., affinity) of the binding protein of interest. Site-directed mutagenesis or PCR-mediated mutagenesis may be performed to introduce the mutation(s) and the effect on protein binding, or other functional property of interest, may be evaluated in in vitro, ex vivo, or in vivo assays as described herein and provided in the Examples. In some embodiments, conservative modifications (as discussed above) may be introduced. The mutations may be amino acid substitutions, additions or deletions. In some embodiments, the mutations are substitutions. Moreover, typically no more than one, two, three, four or five residues within a CDR region are modified.

In some embodiments, binding proteins (e.g., TCRs, antigen-binding fragments of TCRs, or CARs) described herein may possess one or more amino acid substitutions, deletions, or additions relative to a naturally occurring TCR. In some embodiments, each CDR of the binding protein has up to five amino acid substitutions, insertions, deletions, or a combination thereof as compared to the cognate reference CDR sequence listed in Table 2. Conservative substitutions of amino acids are well-known and may occur naturally or may be introduced when the binding protein is recombinantly produced. Amino acid substitutions, deletions, and additions may be introduced into a protein using mutagenesis methods known in the art (see, e.g., Sambrook et al. (2001) Molecular Cloning: A Laboratory Manual, 3d ed., Cold Spring Harbor Laboratory Press, NY). Oligonucleotide-directed site-specific (or segment specific) mutagenesis procedures may be employed to provide an altered polynucleotide that has particular codons altered according to the substitution, deletion, or insertion desired. Alternatively, random or saturation mutagenesis techniques, such as alanine scanning mutagenesis, error prone polymerase chain reaction mutagenesis, and oligonucleotide-directed mutagenesis may be used to prepare immunogen polypeptide variants (see, e.g., Sambrook et al. supra).

A variety of criteria known to the ordinarily skilled artisan indicate whether an amino acid that is substituted at a particular position in a peptide or polypeptide is conservative (or similar). For example, a similar amino acid or a conservative amino acid substitution is one in which an amino acid residue is replaced with an amino acid residue having a similar side chain. Similar amino acids may be included in the following categories: amino acids with basic side chains (e.g., lysine, arginine, histidine); amino acids with acidic side chains (e.g., aspartic acid, glutamic acid); amino acids with uncharged polar side chains (e.g., glycine, asparagine, glutamine, serine, threonine, tyrosine, cysteine, histidine); amino acids with nonpolar side chains (e.g., alanine, valine, leucine, isoleucine, proline, phenylalanine, methionine, tryptophan); amino acids with beta-branched side chains (e.g., threonine, valine, isoleucine), and amino acids with aromatic side chains (e.g., tyrosine, phenylalanine, tryptophan). Proline, which is considered more difficult to classify, shares properties with amino acids that have aliphatic side chains (e.g., leucine, valine, isoleucine, and alanine). In some embodiments, substitution of glutamine for glutamic acid or asparagine for aspartic acid may be considered a similar substitution in that glutamine and asparagine are amide derivatives of glutamic acid and aspartic acid, respectively. As understood in the art “similarity” between two polypeptides is determined by comparing the amino acid sequence and conserved amino acid substitutes thereto of the polypeptide to the sequence of a second polypeptide (e.g., using GENEWORKS™, Align, the BLAST algorithm, or other algorithms described herein and practiced in the art).

In some embodiments, an encoded binding protein (e.g., TCR, antigen-binding fragment of a TCR, or CAR) may comprise a “signal peptide” (also known as a leader sequence, leader peptide, or transit peptide). Signal peptides target newly synthesized polypeptides to their appropriate location inside or outside the cell. A signal peptide may be removed from the polypeptide during or once localization or secretion is completed. Polypeptides that have a signal peptide are referred to herein as a “pre-protein” and polypeptides having their signal peptide removed are referred to herein as “mature” proteins or polypeptides. In some embodiments, a binding protein (e.g., TCR, antigen-binding fragment of a TCR, or CAR) described herein comprises a mature V_{α} domain, a mature V_{β} domain, or both. In some embodiments, a binding protein (e.g., TCR, antigen-binding fragment of a TCR, or CAR) described herein comprises a mature TCR β-chain, a mature TCR α-chain, or both.

In some embodiments, the binding proteins are fusion proteins comprising: (a) an extracellular component comprising a TCR or antigen-binding fragment thereof; (b) an intracellular component comprising an effector domain or a functional portion thereof; and (c) a transmembrane domain connecting the extracellular and intracellular components. In some embodiments, the fusion protein is capable of binding (e.g., specifically and/or selectively) to a peptide-MHC (pMHC) complex comprising a PRAME immunogenic peptide in the context of an MHC molecule (e.g., a MHC class I molecule). In some embodiments, the MHC molecule comprises an MHC alpha chain that is an HLA serotype HLA-A*02. In some embodiments, the HLA allele is selected from the group consisting of HLA-A*02:01, HLA-A*02:02, HLA-A*02:03, HLA-A*02:05, HLA-A*02:06, and HLA-A*02:07 allele. In specific embodiments, the HLA allele is HLA-A*0201.

As used herein, an “effector domain” or “immune effector domain” is an intracellular portion or domain of a fusion protein or receptor that can directly or indirectly promote an immune response in a cell when receiving an appropriate signal. In some embodiments, an effector domain is from an immune cell protein or portion thereof or immune cell protein complex that receives a signal when bound (e.g., CD3ζ), or when the immune cell protein or portion thereof or immune cell protein complex binds directly to a target molecule and triggers signal transduction from the effector domain in an immune cell.

An effector domain may directly promote a cellular response when it contains one or more signaling domains or motifs, such as an intracellular tyrosine-based activation motif (ITAM), such as those found in costimulatory molecules. Without wishing to be bound by theory, it is believed that ITAMs are useful for T cell activation following ligand engagement by a T cell receptor or by a fusion protein comprising a T cell effector domain. In some embodiments, the intracellular component or functional portion thereof comprises an ITAM. Exemplary immune effector domains include but are not limited to those from, CD3ε, CD3δ, CD3ζ, CD25, CD79A, CD79B, CARD11, DAP10, FcRα, FcRβ, FcRγ, Fyn, HVEM, ICOS, Lck, LAG3, LAT, LRP, NKG2D, NOTCH1, NOTCH2, NOTCH3, NOTCH4, Wnt, ROR2, Ryk, SLAMF1, Slp76, pTα, TCRα, TCRβ, TRIM, Zap70, PTCH2, or any combination thereof. In some embodiments, an effector domain comprises a lymphocyte receptor signaling domain (e.g., CD3ζ or a functional portion or variant thereof).

In further embodiments, the intracellular component of the fusion protein comprises a costimulatory domain or a functional portion thereof selected from CD27, CD28, 4-1BB (CD137), OX40 (CD134), CD2, CD5, ICAM-1 (CD54), LFA-1 (CD11a/CD18), ICOS (CD278), GITR, CD30, CD40, BAFF-R, HVEM, LIGHT, MKG2C. SLAMF7, NKp80, CD160, B7-H3, a ligand that binds (e.g., specifically and/or selectively) with CD83, or a functional variant thereof, or any combination thereof. In some embodiments, the intracellular component comprises a CD28 costimulatory domain or a functional portion or variant thereof (which may optionally include a LL-GG mutation at positions 186-187 of the native CD28 protein (e.g., Nguyen et al. (2003) Blood 702:4320), a 4-1BB costimulatory domain or a functional portion or variant thereof, or both.

In some embodiments, an effector domain comprises a CD3ε endodomain or a functional (e.g., signaling) portion thereof, or a functional variant thereof. In further embodiments, an effector domain comprises a CD27 endodomain or a functional (e.g., signaling) portion thereof, or a functional variant thereof. In further embodiments, an effector domain comprises a CD28 endodomain or a functional (e.g., signaling) portion thereof, or a functional variant thereof. In still further embodiments, an effector domain comprises a 4-1BB endodomain or a functional (e.g., signaling) portion thereof, or a functional variant thereof. In further embodiments, an effector domain comprises an OX40 endodomain or a functional (e.g., signaling) portion thereof, or a functional variant thereof. In further embodiments, an effector domain comprises a CD2 endodomain or a functional (e.g., signaling) portion thereof, or a functional variant thereof. In further embodiments, an effector domain comprises a CD5 endodomain or a functional (e.g., signaling) portion thereof, or a functional variant thereof. In further embodiments, an effector domain comprises an ICAM-1 endodomain or a functional (e.g., signaling) portion thereof, or a functional variant thereof. In further embodiments, an effector domain comprises a LFA-1 endodomain or a functional (e.g., signaling) portion thereof, or a functional variant thereof. In further embodiments, an effector domain comprises an ICOS endodomain or a functional (e.g., signaling) portion thereof, or a functional variant thereof.

An extracellular component and an intracellular component encompassed by the present invention are connected by a transmembrane domain. A “transmembrane domain,” as used herein, is a portion of a transmembrane protein that can insert into or span a cell membrane. Transmembrane domains have a three-dimensional structure that is thermodynamically stable in a cell membrane and generally range in length from about 15 amino acids to about 30 amino acids. The structure of a transmembrane domain may comprise an alpha helix, a beta barrel, a beta sheet, a beta helix, or any combination thereof. In some embodiments, the transmembrane domain comprises or is derived from a known transmembrane protein (e.g., a CD4 transmembrane domain, a CD8 transmembrane domain, a CD27 transmembrane domain, a CD28 transmembrane domain, or any combination thereof).

In some embodiments, the extracellular component of the fusion protein further comprises a linker disposed between the binding domain and the transmembrane domain. As used herein when referring to a component of a fusion protein that connects the binding and transmembrane domains, a “linker” may be an amino acid sequence having from about two amino acids to about 500 amino acids, which can provide flexibility and room for conformational movement between two regions, domains, motifs, fragments, or modules connected by the linker. For example, a linker encompassed by the present invention can position the binding domain away from the surface of a host cell expressing the fusion protein to enable proper contact between the host cell and a target cell, antigen binding, and activation (Patel et al. (1999) Gene Therapy 6:412-419). Linker length may be varied to maximize antigen recognition based on the selected target molecule, selected binding epitope, or antigen binding domain seize and affinity (see, e.g., Guest et al. (2005) Immunother. 28:203-11 and PCT Publ. No. WO 2014/031687). Exemplary linkers include those having a glycine-serine amino acid chain having from one to about ten repeats of GlyₓSer_{y}, wherein x and y are each independently an integer from 0 to 10, provided that x and y are not both 0 (e.g., (Gly₄Ser)₂, (Gly₃Ser)₂, Gly₂Ser, or a combination thereof, such as ((Gly₃Ser)₂Gly₂Ser)).

A binding protein may be conjugated to an agent, such as a detection moiety, radiosensitizer, photosensitizer, and the like, and/or may be chemically modified as described above regarding peptides.

Binding proteins encompassed by the present invention may, in some embodiments, be covalently linked to a moiety. In some embodiments, the covalently linked moiety comprises an affinity tag or a label. The affinity tag may be selected from the group consisting of Glutathione-S-Transferase (GST), calmodulin binding protein (CBP), protein C tag, Myc tag, HaloTag, HA tag, Flag tag, His tag, biotin tag, and V5 tag. The label may be a fluorescent protein. In some embodiments, the covalently linked moiety is selected from the group consisting of an inflammatory agent, an anti-inflammatory agent, a cytokine, a toxin, a cytotoxic molecule, a radioactive isotope, or an antibody such as a single-chain Fv.

A binding protein may be conjugated to an agent used in imaging, research, therapeutics, theranostics, pharmaceuticals, chemotherapy, chelation therapy, targeted drug delivery, and radiotherapy. In some embodiments, a binding protein may be conjugated to or fused with detectable agents, such as a fluorophore, a near-infrared dye, a contrast agent, a nanoparticle, a metal-containing nanoparticle, a metal chelate, an X-ray contrast agent, a PET agent, a metal, a radioisotope, a dye, radionuclide chelator, or another suitable material that can be used in imaging. In some embodiments, 1, 2, 3, 4, 5, 6, 7, 8, 9, 10, or more detectable moieties may be linked to a binding protein. Non-limiting examples of radioisotopes include alpha emitters, beta emitters, positron emitters, and gamma emitters. In some embodiments, the metal or radioisotope is selected from the group consisting of actinium, americium, bismuth, cadmium, cesium, cobalt, europium, gadolinium, iridium, lead, lutetium, manganese, palladium, polonium, radium, ruthenium, samarium, strontium, technetium, thallium, and yttrium. In some embodiments, the metal is actinium, bismuth, lead, radium, strontium, samarium, or yttrium. In some embodiments, the radioisotope is actinium-225 or lead-212 In some embodiments, the near-infrared dyes are not easily quenched by biological tissues and fluids. In some embodiments, the fluorophore is a fluorescent agent emitting electromagnetic radiation at a wavelength between 650 nm and 4000 nm, such emissions being used to detect such agent. Non-limiting examples of fluorescent dyes that may be used as a conjugating molecule include DyLight-680, DyLight-750, VivoTag-750, DyLight-800, IRDye-800, VivoTag-680, Cy5.5, ZQ800, or indocyanine green (ICG). In some embodiments, near infrared dyes often include cyanine dyes (e.g., Cy7, Cy5.5, and Cy5). Additional, non-limiting examples of fluorescent dyes for use as a conjugating molecule in accordance with present invention include acridine orange or yellow, Alexa Fluors® (e.g., Alexa Fluor®790, 750, 700, 680, 660, and 647) and any derivative thereof, 7-actinomycin D, 8-anilinonaphthalene-1-sulfonic acid, ATTO® dye and any derivative thereof, auramine-rhodamine stain and any derivative thereof, benzanthrone, bimane, 9-10-bis(phenylethynyl)anthracene, 5,12-bis(phenylethynyl)naphthacene, bisbenzimide, brainbow, calcein, carboxyfluorescein and any derivative thereof, 1-chloro-9,10-bis(phenylethynyl)anthracene and any derivative thereof, DAPI, DiOC6. DyLight® Fluors® and any derivative thereof, epicocconone, ethidium bromide, FlAsH-EDT2@, Fluo dye and any derivative thereof, FluoProbe® and any derivative thereof, fluorescein and any derivative thereof, Fura® and any derivative thereof, GelGreen® and any derivative thereof, GelRed® and any derivative thereof, fluorescent proteins and any derivative thereof, m isoform proteins and any derivative thereof such as for example mCherry, heptamethine dye and any derivative thereof, hoeschst stain, iminocoumarin, indian yellow, indo-1 and any derivative thereof, laurdan, lucifer yellow and any derivative thereof, luciferin and any derivative thereof, luciferase and any derivative thereof, merocyanine and any derivative thereof, nile dyes and any derivative thereof, perylene, phloxine, phyco dye and any derivative thereof, propium iodide, pyranine, rhodamine and any derivative thereof, ribogreen. RoGFP, rubrene, stilbene and any derivative thereof, sulforhodamine and any derivative thereof, SYBR and any derivative thereof, synapto-pHluorin, tetraphenyl butadiene, tetrasodium tris, Texas Red, Titan Yellow, TSQ, umbelliferone, violanthrone, yellow fluorescent protein and YOYO-1. Other suitable fluorescent dyes include, but are not limited to, fluorescein and fluorescein dyes (e.g., fluorescein isothiocyanine or FITC, naphthofluorescein, 4′,5′-dichloro-2′,7′-dimethoxyfluorescein, 6-carboxyfluorescein or FAM, etc.), carbocyanine, merocyanine, styryl dyes, oxonol dyes, phycoerythrin, erythrosin, eosin, rhodamine dyes (e.g., carboxytetramethyl-rhodamine or TAMRA, carboxyrhodamine 6G, carboxy-X-rhodanine (ROX), lissamine rhodamine B. rhodamine 6G, rhodamine Green, rhodamine Red, tetramethylrhodamine (TMR), etc.), coumarin and coumarin dyes (e.g., methoxycoumarin, dialkylaminocoumarin, hydroxycoumarin, aminomethylcoumarin (AMCA), etc.), Oregon Green™ dyes (e.g., Oregon Green™ 488, 500, 514, etc.), Texas Red®, Texas Red®-X. SPECTRUM RED®, SPECTRUM GREEN®, cyanine dyes (e.g., CY-3, Cy-5, CY-3.5, CY-5.5, etc.). Alexa Fluor® dyes (e.g., Alexa Fluor®350, 488, 532, 546, 568, 594, 633, 660, 680, etc.), BODIPY® dyes (e.g., BODIPY® FL, R6G, TMR, TR, 530/550, 558/568, 564/570, 576/589, 581/591, 630/650, 650/665, etc.), IRD dyes (e.g., IRD40™. IRD700™, IRD800™, etc.), and the like. Additional suitable detectable agents are well-known in the art (e.g., PCT Publ. No. PCT/US14/56177). Non-limiting examples of radioisotopes include alpha emitters, beta emitters, positron emitters, and gamma emitters. In some embodiments, the metal or radioisotope is selected from the group consisting of actinium, americium, bismuth, cadmium, cesium, cobalt, europium, gadolinium, iridium, lead, lutetium, manganese, palladium, polonium, radium, ruthenium, samarium, strontium, technetium, thallium, and yttrium. In some embodiments, the metal is actinium, bismuth, lead, radium, strontium, samarium, or yttrium. In some embodiments, the radioisotope is actinium-225 or lead-212.

Binding proteins may be conjugated to a radiosensitizer or photosensitizer. Examples of radiosensitizers include but are not limited to: ABT-263, ABT-199, WEHI-539, paclitaxel, carboplatin, cisplatin, oxaliplatin, gemcitabine, etanidazole, misonidazole, tirapazamine, and nucleic acid base derivatives (e.g., halogenated purines or pyrimidines, such as 5-fluorodeoxyuridine). Examples of photosensitizers include but are not limited to: fluorescent molecules or beads that generate heat when illuminated, nanoparticles, porphyrins and porphyrin derivatives (e.g., chlorins, bacteriochlorins, isobacteriochlorins, phthalocyanines, and naphthalocyanines), metalloporphyrins, metallophthalocyanines, angeicins, chalcogenapyrylium dyes, chlorophylls, coumarins, flavins and related compounds such as alloxazine and riboflavin, fullerenes, pheophorbides, pyropheophorbides, cyanines (e.g., merocyanine 540), pheophytins, sapphyrins, texaphyrins, purpurins, porphycenes, phenothiaziniums, methylene blue derivatives, naphthalimides, nile blue derivatives, quinones, perylenequinones (e.g., hypericins, hypocrellins, and cercosporins), psoralens, quinones, retinoids, rhodamines, thiophenes, verdins, xanthene dyes (e.g., eosins, erythrosins, rose bengals), dimeric and oligomeric forms of porphyrins, and prodrugs such as 5-aminolevulinic acid. Advantageously, this approach allows for highly specific targeting of cells of interest (e.g., immune cells) using both a therapeutic agent (e.g., drug) and electromagnetic energy (e.g., radiation or light) concurrently. In some embodiments, the binding protein is fused with, or covalently or non-covalently linked to the agent, for example, directly or via a linker.

In some embodiments, the binding protein may be chemically modified. For example, a binding protein may be mutated to modify peptide properties such as detectability, stability, biodistribution, pharmacokinetics, half-life, surface charge, hydrophobicity, conjugation sites, pH, function, and the like. N-methylation is one example of methylation that can occur in a binding protein encompassed by the present invention. In some embodiments, a binding protein may be modified by methylation on free amines such as by reductive methylation with formaldehyde and sodium cyanoborohydride.

A chemical modification may comprise a polymer, a polyether, polyethylene glycol, a biopolymer, a zwitterionic polymer, a polyamino acid, a fatty acid, a dendrimer, an Fc region, a simple saturated carbon chain such as palmitate or myristoleate, or albumin. The chemical modification of a binding protein with an Fc region may be a fusion Fc-protein. A polyamino acid may include, for example, a poly amino acid sequence with repeated single amino acids (e.g., poly glycine), and a poly amino acid sequence with mixed poly amino acid sequences that may or may not follow a pattern, or any combination of the foregoing.

In some embodiments, the binding proteins encompassed by the present invention may be modified. In some embodiments, the modifications having substantial or significant sequence identity to a parent binding protein to generate a functional variant that maintains one or more biophysical and/or biological activities of the parent binding protein (e.g., maintain pMHC binding specificity). In some embodiments, the mutation is a conservative amino acid substitution.

In some embodiments, binding proteins encompassed by the present invention may comprise synthetic amino acids in place of one or more naturally-occurring amino acids. Such synthetic amino acids are well-known in the art, and include, for example, aminocyclohexane carboxylic acid, norleucine, α-amino n-decanoic acid, homoserine, S-acetylaminomethyl-cysteine, trans-3- and trans-4-hydroxyproline, 4-aminophenylalanine, 4-nitrophenylalanine, 4-chlorophenylalanine, 4-carboxyphenylalanine, β-phenylserine β-hydroxyphenylalanine, phenylglycine, α-naphthylalanine, cyclohexylalanine, cyclohexylglycine, indoline-2-carboxylic acid, 1,2,3,4-tetrahydroisoquinoline-3-carboxylic acid, aminomalonic acid, aminomalonic acid monoamide, N′-benzyl-N′-methyl-lysine, N′,N′-dibenzyl-lysine, 6-hydroxylysine, ornithine, α-aminocyclopentane carboxylic acid, oc-aminocyclohexane carboxylic acid, α-aminocycloheptane carboxylic acid. α-(2-amino-2-norbornane)-carboxylic acid, α,γ-diaminobutyric acid, β-diaminopropionic acid, homophenylalanine, and oc-tert-butylglycine.

Binding proteins encompassed by the present invention may be glycosylated, amidated, carboxylated, phosphorylated, esterified, N-acylated, cyclized (e.g., via a disulfide bridge), or converted into an acid addition salt and/or optionally dimerized or polymerized, or conjugated.

In some embodiments, the attachment of a hydrophobic moiety, such as to the N-terminus, the C-terminus, or an internal amino acid, may be used to extend half-life of a peptide encompassed by the present invention. In other embodiments, a binding protein may include post-translational modifications (e.g., methylation and/or amidation), which can affect, for example, serum half-life. In some embodiments, simple carbon chains (e.g., by myristoylation and/or palmitoylation) may be conjugated to the binding proteins. In some embodiments, the simple carbon chains may render the binding proteins easily separable from the unconjugated material. For example, methods that may be used to separate the binding proteins from the unconjugated material include, but are not limited to, solvent extraction and reverse phase chromatography. The lipophilic moieties can extend half-life through reversible binding to serum albumin. The conjugated moieties may be lipophilic moieties that extend half-life of the peptides through reversible binding to serum albumin. In some embodiments, the lipophilic moiety may be cholesterol or a cholesterol derivative, including cholestenes, cholestanes, cholestadienes and oxysterols. In some embodiments, the binding proteins may be conjugated to myristic acid (tetradecanoic acid) or a derivative thereof. In other embodiments, a binding protein may be coupled (e.g., conjugated) to a half-life modifying agent. Examples of half-life modifying agents include but are not limited to: a polymer, a polyethylene glycol (PEG), a hydroxyethyl starch, polyvinyl alcohol, a water soluble polymer, a zwitterionic water soluble polymer, a water soluble poly(amino acid), a water soluble polymer of proline, alanine and serine, a water soluble polymer containing glycine, glutamic acid, and serine, an Fc region, a fatty acid, palmitic acid, or a molecule that binds to albumin. In some embodiments, a spacer or linker may be coupled to a binding protein, such as 1, 2, 3, 4, or more amino acid residues that serve as a spacer or linker in order to facilitate conjugation or fusion to another molecule, as well as to facilitate cleavage of the peptide from such conjugated or fused molecules. In some embodiments, binding proteins may be conjugated to other moieties that, for example, can modify or effect changes to the properties of the binding proteins.

A binding protein may be produced recombinantly or synthetically, such as by solid-phase peptide synthesis or solution-phase peptide synthesis. Polypeptide synthesis may be performed by known synthetic methods, such as using fluorenylmethyloxycarbonyl (Fmoc) chemistry or by butyloxycarbonyl (Boc) chemistry. Polypeptide fragments may be joined together enzymatically or synthetically.

In an aspect encompassed by the present invention, provided herein are methods of producing a binding protein described herein, comprising the steps of: (i) culturing a transformed host cell which has been transformed by a nucleic acid comprising a sequence encoding a binding protein described herein under conditions suitable to allow expression of said binding protein; and (ii) recovering the expressed binding protein.

Methods useful for isolating and purifying recombinantly produced binding protein, by way of example, may include obtaining supernatants from suitable host cell/vector systems that secrete the binding protein into culture media and then concentrating the media using a commercially available filter. Following concentration, the concentrate may be applied to a single suitable purification matrix or to a series of suitable matrices, such as an affinity matrix or an ion exchange resin. One or more reverse phase HPLC steps may be employed to further purify a recombinant polypeptide. These purification methods may also be employed when isolating an immunogen from its natural environment. Methods for large scale production of one or more of binding proteins described herein include batch cell culture, which is monitored and controlled to maintain appropriate culture conditions. Purification of the binding protein may be performed according to methods described herein and known in the art.

In any of the herein disclosed embodiments, the encoded binding protein is capable of bind to a peptide-MHC (pMHC) complex comprising a PRAME immunogenic peptide in the context of an MHC molecule (e.g., a MHC class I molecule). In some embodiments, the MHC molecule comprises an MHC alpha chain that is an HLA serotype HLA-A*02. In some embodiments, the HLA allele is selected from the group consisting of HLA-A*0201, HLA-A*0202, HLA-A*0203, HLA-A*0205, HLA-A*0206, and HLA-A*0207 allele.

A variety of assays are well-known for assessing binding affinity and/or determining whether a binding molecule binds (e.g., specifically and/or selectively) to a particular ligand (e.g., peptide antigen-MHC complex). It is within the level of a skilled artisan to determine the binding affinity of a binding protein for a target, such as a T cell peptide epitope of a target polypeptide, such as by using any of a number of binding assays that are well-known in the art. For example, in some embodiments, a Biacore™ machine may be used to determine the binding constant of a complex between two proteins. The dissociation constant (K) for the complex may be determined by monitoring changes in the refractive index with respect to time as buffer is passed over the chip. Other suitable assays for measuring the binding of one protein to another include, for example, immunoassays such as enzyme linked immunosorbent assays (ELISA) and radioimmunoassays says (RIA), or determination of binding by monitoring the change in the spectroscopic or optical properties of the proteins through fluorescence, UV absorption, circular dichroism, or nuclear magnetic resonance (NMR). Other exemplary assays include, but are not limited to, Western blot, ELISA, analytical ultracentrifugation, spectroscopy and surface plasmon resonance (Biacore™) analysis (see, e.g., Scatchard et al. (1949) Ann. N.Y. Acad. Sci, 51:660, Wilson (2002) Science 295:2103, Wolff et al. (1993) Cancer Res, 53:2560, and U.S. Pat. Nos. 5,283,173 and 5,468,614), flow cytometry, sequencing and other methods for detection of expressed nucleic acids. In one example, apparent affinity for a target is measured by assessing binding to various concentrations of tetramers, for example, by flow cytometry using labeled multimers, such as MHC-antigen tetramers. In one representative example, apparent K_{D} of a binding protein is measured using 2-fold dilutions of labeled tetramers at a range of concentrations, followed by determination of binding curves by non-linear regression, apparent K_{D} being determined as the concentration of ligand that yielded half-maximal binding.

### III. Nucleic Acids and Vectors

In an aspect encompassed by the present invention, provided herein are nucleic acid molecules that encode proteins described herein, such as PRAME immunogenic peptides and fragments thereof, MHC molecules, binding proteins (e.g., TCRs, antigen-binding fragments of the TCRs, CARs, and the like), and the like.

In some embodiments, the nucleic acid molecule hybridizes, under stringent conditions, with the complement of a sequence with at least about at least about 80%, 81%, 82%, 83%, 84%, 85%, 86%, 87%, 88%, 89%, 90%, 91%, 92%, 93%, 94%, 95%, 96%, 97%, 98%, 99%, or more identity, such as over the full length, to a nucleic acid encoding a polypeptide selected from the group consisting of the polypeptide sequences listed in Tables 1-4.

In some embodiments, the nucleic acid molecule hybridizes, under stringent conditions, with the complement of a nucleic acid encoding a polypeptide selected from the group consisting of polypeptide sequences listed in Tables 1-4.

In some embodiments, the nucleic acid molecule comprises (e.g., comprises, consists essentially of, or consists of) a nucleotide sequence encoding a polypeptide selected from the group consisting of polypeptide sequences listed in Tables 1-4.

In some embodiments, the nucleic acid sequence encodes a PRAME immunogenic peptides described herein.

In some embodiments, the nucleic acids comprise (e.g., comprise, consist essentially of, or consist of) a nucleotide sequence encoding at least one (e.g., one, two, or three) TCR α-chain CDR set forth in Table 2. In some embodiments, the nucleic acids comprise (e.g., comprise, consist essentially of, or consist of) a nucleotide sequence encoding a TCR V_{α} domain having an amino acid sequence that is at least about at least about 80%, 81%, 82%, 83%, 84%, 85%, 86%, 87%, 88%, 89%, 90%, 91%, 92%, 93%, 94%, 95%, 96%, 97%, 98%, 99%, or more identity to a TCR V_{α} domain sequence set forth in Table 2. In some embodiments, the nucleic acids comprise (e.g., comprise, consist essentially of, or consist of) a nucleotide sequence encoding a TCR α-chain having an amino acid sequence that is at least about at least about 80%, 81%, 82%, 83%, 84%, 85%, 86%, 87%, 88%, 89%, 90%, 91%, 92%, 93%, 94%, 95%, 96%, 97%, 98%, 99%, or more identity to a TCR α-chain sequence set forth in Table 2.

In some embodiments, the nucleic acids comprise (e.g., comprise, consist essentially of, or consist of) a nucleotide sequence encoding at least one (e.g., one, two, or three) TCR β-chain CDR set forth in Table 2. In some embodiments, the nucleic acids comprise (e.g., comprise, consist essentially of, or consist of) a nucleotide sequence encoding a TCR V_{β} domain having an amino acid sequence that is at least about at least about 80%, 81%, 82%, 83%, 84%, 85%, 86%, 87%, 88%, 89%, 90%, 91%, 92%, 93%, 94%, 95%, 96%, 97%, 98%, 99%, or more identity to a TCR V_{β} domain sequence set forth in Table 2. In some embodiments, the nucleic acids comprise (e.g., comprise, consist essentially of, or consist of) a nucleotide sequence encoding a TCR β-chain having an amino acid sequence that is at least about at least about 80%, 81%, 82%, 83%, 84%, 85%, 86%, 87%, 88%, 89%, 90%, 91%, 92%, 93%, 94%, 95%, 96%, 97%, 98%, 99%, or more identity to a TCR β-chain sequence set forth in Table 2.

The term “nucleic acid” includes “polynucleotide,” “oligonucleotide,” and “nucleic acid molecule,” and generally means a polymer of DNA or RNA, which may be single-stranded or double-stranded, synthesized or obtained (e.g., isolated and/or purified) from natural sources, which may contain natural, non-natural or altered nucleotides, and which may contain a natural, non-natural or altered internucleotide linkage, such as a phosphoroamidate linkage or a phosphorothioate linkage, instead of the phosphodiester found between the nucleotides of an unmodified oligonucleotide. In an embodiment, the nucleic acid comprises complementary DNA (cDNA).

In some embodiments, the nucleic acids encompassed by the present invention are recombinant. As used herein, the term “recombinant” refers to (i) molecules that are constructed outside living cells by joining natural or synthetic nucleic acid segments to nucleic acid molecules that may replicate in a living cell, or (ii) molecules that result from the replication of those described in (i) above. For purposes herein, the replication may be in vitro/ex vivo replication or in vivo replication.

The nucleic acids can be constructed based on chemical synthesis and/or enzymatic ligation reactions using procedures known in the art. See, for example, Green and Sambrook et al. supra. For example, a nucleic acid may be chemically synthesized using naturally occurring nucleotides or variously modified nucleotides designed to increase the biological stability of the molecules or to increase the physical stability of the duplex formed upon hybridization (e.g., phosphorothioate derivatives and acridine substituted nucleotides). Examples of modified nucleotides that may be used to generate the nucleic acids include, but are not limited to, 5-fluorouracil, 5-bromouracil, 5-chlorouracil, 5-iodouracil, hypoxanthine, xanthine, 4-acetylcytosine, 5-(carboxyhydroxymethyl) uracil, 5-carboxymethylaminomethyl-2-thiouridine, 5-carboxymethylaminomethyluracil, dihydrouracil, beta-D-galactosylqueosine, inosine, N⁶-isopentenyladenine, 1-methylguanine, 1-methylinosine, 2,2-dimethylguanine, 2-methyladenine, 2-methylguanine, 3-methylcytosine, 5-methylcytosine, N⁶-substituted adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N⁶-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, 3-(3-amino-3-N-2-carboxypropyl) uracil, and 2,6-diaminopurine. Alternatively, one or more of the nucleic acids encompassed by the present invention can be purchased from companies, such as Integrated DNA Technologies (Coralville, IA).

In one embodiment, the nucleic acid comprises a codon-optimized nucleotide sequence. Without being bound to a particular theory or mechanism, it is believed that codon optimization of the nucleotide sequence increases the translation efficiency of the mRNA transcripts. Codon optimization of the nucleotide sequence may involve substituting a native codon for another codon that encodes the same amino acid, but can be translated by tRNA that is more readily available within a cell, thus increasing translation efficiency. Optimization of the nucleotide sequence may also reduce secondary mRNA structures that would interfere with translation, thus increasing translation efficiency. In some embodiments, the nucleotide sequences described herein are codon-optimized for expression in a host cell (e.g., an immune cell, such as a T cell).

The present invention also provides a nucleic acid comprising a nucleotide sequence which is complementary to the nucleotide sequence of any of the nucleic acids described herein or a nucleotide sequence which hybridizes under stringent conditions to the nucleotide sequence of any of the nucleic acids described herein.

The nucleotide sequence which hybridizes under stringent conditions may hybridize under high stringency conditions. By “high stringency conditions” is meant that the nucleotide sequence specifically hybridizes to a target sequence (the nucleotide sequence of any of the nucleic acids described herein) in an amount that is detectably stronger than non-specific hybridization. High stringency conditions include conditions which would distinguish a polynucleotide with an exact complementary sequence, or one containing only a few scattered mismatches from a random sequence that happened to have a few small regions (e.g., 3-10 bases) that matched the nucleotide sequence. Such small regions of complementarity are more easily melted than a full-length complement of 14-17 or more bases, and high stringency hybridization makes them easily distinguishable. Relatively high stringency conditions would include, for example, low salt and/or high temperature conditions, such as provided by about 0.02-0.1 M NaCl or the equivalent, at temperatures of about 50-70° C. Such high stringency conditions tolerate little, if any, mismatch between the nucleotide sequence and the template or target strand, and are particularly suitable for detecting expression of any of the inventive TCRs. It is generally appreciated that conditions may be rendered more stringent by the addition of increasing amounts of formamide.

The present invention also provides a nucleic acid comprising a nucleotide sequence that is at least about 80%, 81%, 82%, 83%, 84%, 85%, 86%, 87%, 88%, 89%, 90%, 91%, 92%, 93%, 94%, 95%, 96%, 97%, 98%, 99%, or more identical to any of the nucleic acids described herein.

Typically, said nucleic acid is a DNA or RNA molecule, which may be included in a suitable vector, such as a plasmid, cosmid, episome, artificial chromosome, phage or a viral vector.

The terms “vector”, “cloning vector” and “expression vector” mean the vehicle by which a DNA or RNA sequence (e.g., a foreign gene) can be introduced into a host cell, so as to transform the host and promote expression (e.g., transcription and translation) of the introduced sequence. Thus, a further object encompassed by the present invention relates to a vector comprising a nucleic acid encompassed by the present invention.

Such vectors may comprise regulatory elements, such as a promoter, enhancer, terminator and the like, to cause or direct expression of said polypeptide upon administration to a subject. Examples of promoters and enhancers used in the expression vector for animal cell include early promoter and enhancer of SV40 (Mizukami T. et al, 1987), LTR promoter and enhancer of Moloney mouse leukemia virus (Kuwana Y et al, 1987), promoter (Mason J O et al, 1985) and enhancer (Gillies S D et al, 1983) of immunoglobulin H chain and the like.

Any expression vector for animal cell may be used. Examples of suitable vectors include pAGE107 (Miyaji H et al, 1990), pAGE103 (Mizukami T et al, 1987), pHSG274 (Brady G et al, 1984), pKCR (O'Hare K et al, 1981), pSG1 beta d2-4—(Miyaji H et al, 1990) and the like. Other representative examples of plasmids include replicating plasmids comprising an origin of replication, or integrative plasmids, such as for instance pUC, pcDNA, pBR, and the like. Representative examples of viral vector include adenoviral, retroviral, lentiviral, herpes virus and AAV vectors. Such recombinant viruses may be produced by techniques known in the art, such as by transfecting packaging cells or by transient transfection with helper plasmids or viruses. Typical examples of virus packaging cells include PA317 cells, PsiCRIP cells, GPenv-positive cells, 293 cells, etc. Detailed protocols for producing such replication-defective recombinant viruses are well-known in the art and may be found, for instance, in PCT Publ. WO 95/14785, PCT. Publ. WO 96/22378, U.S. Pat. Nos. 5,882,877, 6,013,516, 4,861,719, 5,278,056, and PCT Publ. WO 94/19478.

In some embodiments, the composition comprises an expression vector comprising an open reading frame encoding a binding protein or a polypeptide described herein or a fragment thereof. In some embodiments, the nucleic acid includes regulatory elements necessary for expression of the open reading frame. Such elements may include, for example, a promoter, an initiation codon, a stop codon, and a polyadenylation signal. In addition, enhancers may be included. These elements may be operably linked to a sequence that encodes the binding protein, polypeptide or fragment thereof.

In some embodiments, the vector further comprises a nucleic acid sequence encoding CD8α, CD8β, a dominant negative TGFβ receptor (e.g., a DN-TGFβRII), selectable protein marker, optionally wherein the selectable protein marker is dihydrofolate reductase (DHFR). In certain embodiments, the nucleic acid sequence encoding CD8α, CD8β, the DN-TGFβR, and/or the selectable protein marker is operably linked to a nucleic acid encoding a tag (e.g., a CD34 enrichment tag). In specific embodiments, a nucleic acid sequence described herein, such as a nucleic acid sequence encoding a TCRα, TCRβ, CD8α, CD8β, DN-TGFβR, and/or selectable protein marker are interconnected with an internal ribosome entry site or a nucleic acid sequence encoding a self-cleaving peptide, such as P2A, E2A, F2A or T2A, etc.

In some embodiments, the expression vector provided herein comprises a nucleotide sequence that is at least about 80%, 81%, 82%, 83%, 84%, 85%, 86%, 87%, 88%, 89%, 90%, 91%, 92%, 93%, 94%, 95%, 96%, 97%, 98%, 99%, or more identical to any of the nucleic acids set forth in Tables 1-3.

Examples of promoters include, but are not limited to, promoters from Simian Virus 40 (SV40), Mouse Mammary Tumor Virus (MMTV) promoter. Human Immunodeficiency Virus (HIV) such as the HIV Long Terminal Repeat (LTR) promoter, Moloney virus, Cytomegalovirus (CMV) such as the CMV immediate early promoter, Epstein Barr Virus (EBV), Rous Sarcoma Virus (RSV) as well as promoters from human genes such as human actin, human myosin, human hemoglobin, human muscle creatine, and human metallothionein. Examples of suitable polyadenylation signals include but are not limited to SV40 polyadenylation signals and LTR polyadenylation signals.

In addition to the regulatory elements required for expression, other elements may also be included in the nucleic acid molecule. Such additional elements include enhancers. Enhancers include the promoters described herein. In some embodiments, enhancers/promoters include, for example, human actin, human myosin, human hemoglobin, human muscle creatine and viral enhancers such as those from CMV, RSV and EBV.

In some embodiments, the nucleic acid may be operably incorporated in a carrier or delivery vector as described further below. Useful delivery vectors include but are not limited to biodegradable microcapsules, immuno-stimulating complexes (ISCOMs) or liposomes, and genetically engineered attenuated live carriers such as viruses or bacteria.

In some embodiments, the vector is a viral vector, such as lentiviruses, retroviruses, herpes viruses, adenoviruses, adeno-associated viruses, vaccinia viruses, baculoviruses. Fowl pox. AV-pox, modified vaccinia Ankara (MVA) and other recombinant viruses. For example, a lentivirus vector may be used to infect T cells.

In some embodiments, the recombinant expression vector is capable of delivering a polynucleotide to an appropriate host cell, for example, a T cell or an antigen-presenting cell, i.e., a cell that displays a peptide/MHC complex on its cell surface (e.g., a dendritic cell) and lacks CD8. In some embodiments, the host cell is a hematopoietic progenitor cell or a human immune system cell. For example, the immune system cell may be a CD4⁺ T cell, a CD8+ T cell, a CD4/CD8 double negative T cell, a gd T cell, a natural killer cell, a dendritic cell, or any combination thereof. In some embodiments, wherein a T cell is the host, the T cell may be naive, a central memory T cell, an effector memory T cell, or any combination thereof. The recombinant expression vectors may therefore also include, for example, lymphoid tissue-specific transcriptional regulatory elements (TREs), such as a B lymphocyte, T lymphocyte, or dendritic cell specific TREs. Lymphoid tissue specific TREs are known in the art (see, e.g., Thompson et al. (1992) Mol. Cell. Biol, 72:1043. Todd et al. (1993) J. Exp. Med. 777:1663, and Penix et al. (1993) J. Exp. Med, 775:1483).

In some embodiments, a recombinant expression vector comprises a nucleotide sequence encoding a TCR α chain, a TCR β chain, and/or a linker peptide. For example, in some embodiments, the recombinant expression vector comprises a nucleotide sequence encoding the full-length TCR alpha and TCR beta chains of the binding protein with a linker positioned between them, wherein the nucleotide sequence encoding the beta chain is positioned 5′ of the nucleotide sequence encoding the alpha chain. In some embodiments, the nucleotide sequence encodes the full-length TCR alpha and TCR beta chains with a linker positioned between them, wherein the nucleotide sequence encoding the TCR beta chain is positioned 3′ of the nucleotide sequence encoding the TCR alpha chain. In some embodiments, the full-length TCR alpha and/or TCR beta chains are replaced with fragments thereof.

As described further below, another aspect encompassed by the present invention relates to a cell which has been transfected, infected or transformed by a nucleic acid and/or a vector in accordance with the present invention. A host cell may include any individual cell or cell culture which may receive a vector or the incorporation of nucleic acids and/or proteins, as well as any progeny cells. The term also encompasses progeny of the host cell, whether genetically or phenotypically the same or different. Suitable host cells may depend on the vector and may include mammalian cells, animal cells, human cells, simian cells, insect cells, yeast cells, and bacterial cells. These cells may be induced to incorporate the vector or other material by use of a viral vector, transformation via calcium phosphate precipitation, DEAE-dextran, electroporation, microinjection, or other methods (see, e.g., Sambrook et al. (1989) Molecular Cloning: A Laboratory Manual 2d ed. (Cold Spring Harbor Laboratory)). The term “transformation” means the introduction of a “foreign” (i.e., extrinsic or extracellular) gene, DNA or RNA sequence to a host cell, so that the host cell will express the introduced gene or sequence to produce a desired substance, typically a protein or enzyme coded by the introduced gene or sequence. A host cell that receives and expresses introduced DNA or RNA has been “transformed.”

The nucleic acids encompassed by the present invention may be used to produce a recombinant polypeptide encompassed by the present invention in a suitable expression system. The term “expression system” means a host cell and compatible vector under suitable conditions, e.g., for the expression of a protein coded for by foreign DNA carried by the vector and introduced to the host cell.

Common expression systems include E. coli host cells and plasmid vectors, insect host cells and Baculovirus vectors, and mammalian host cells and vectors. Other examples of host cells include, without limitation, prokaryotic cells (such as bacteria) and eukaryotic cells (such as yeast cells, mammalian cells, insect cells, plant cells, etc.). Specific examples include E. coli, Kluyveromyces or Saccharomyces yeasts, mammalian cell lines (e.g., Vero cells, CHO cells, 3T3 cells, COS cells, etc.) as well as primary or established mammalian cell cultures (e.g., produced from lymphoblasts, fibroblasts, embryonic cells, epithelial cells, nervous cells, adipocytes, etc.). Examples also include mouse SP2/0-Ag14 cell (ATCC CRL1581), mouse P3X63-Ag8.653 cell (ATCC CRL1580), CHO cell in which a dihydrofolate reductase gene (hereinafter referred to as “DHFR gene”) is defective (Urlaub G et al (1980), rat YB2/3HL.P2.G11.16Ag.20 cell (ATCC CRL 1662, hereinafter referred to as “YB2/0 cell”), and the like. In some embodiments, the YB2/0 cell is used since ADCC activity of chimeric or humanized binding proteins is enhanced when expressed in this cell.

The present invention also encompasses methods of producing a recombinant host cell expressing binding proteins, peptides and fragments thereof encompassed by the present invention, said method comprising the steps consisting of (i) introducing in vitro or ex vivo a recombinant nucleic acid or a vector as described above into a competent host cell, (ii) culturing in vitro or ex vivo the recombinant host cell obtained and (iii), optionally, selecting the cells which express said binding proteins, peptides and fragments thereof. Such recombinant host cells may be used for the diagnostic, prognostic, and/or therapeutic method encompassed by the present invention.

In another aspect, the present invention provides isolated nucleic acids that hybridize under selective hybridization conditions to a polynucleotide disclosed herein. Thus, the polynucleotides of this embodiment may be used for isolating, detecting, and/or quantifying nucleic acids comprising such polynucleotides. For example, polynucleotides encompassed by the present invention may be used to identify, isolate, or amplify partial or full-length clones in a deposited library. In some embodiments, the polynucleotides are genomic or cDNA sequences isolated, or otherwise complementary to, a cDNA from a human or mammalian nucleic acid library. In some embodiments, the cDNA library comprises at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more, or any range in between, inclusive, such as at least about 80%-100%, full-length sequences. The cDNA libraries may be normalized to increase the representation of rare sequences. Low or moderate stringency hybridization conditions are typically, but not exclusively, employed with sequences having a reduced sequence identity relative to complementary sequences. Moderate and high stringency conditions may optionally be employed for sequences of greater identity. Low stringency conditions allow selective hybridization of sequences having about 70% sequence identity and may be employed to identify orthologous or paralogous sequences. Optionally, polynucleotides encompassed by the present invention will encode at least a portion of a binding protein encoded by the polynucleotides described herein. The polynucleotides encompassed by the present invention embrace nucleic acid sequences that may be employed for selective hybridization to a polynucleotide encoding a binding protein encompassed by the present invention (see, e.g., Ausubel, supra and Colligan, supra).

### IV. Host Cells

In an aspect encompassed by the present invention, provided herein are host cells that express proteins described herein, such as PRAME immunogenic peptides, PRAME immunogenic peptide-MHC (pMHC) complexes, PRAME binding proteins (e.g., TCRs, antigen-binding fragments of TCRs, CARs, or fusion proteins comprising a TCR and an effector domain), and the like described herein. In some embodiments, the host cells comprise the nucleic acids or vectors described herein.

In some embodiments, a polynucleotide encoding a binding protein is used to transform, transfect, or transduce a host cell (e.g., a T cell) for use in adoptive transfer therapy. Advances in nucleic acid sequencing and particular TCR sequencing have been described (e.g., Robins et al. (2009) Blood 114:4099; Robins et al. (2010) Sci. Translat. Med 2:47ra64, Robins et al. (2011) J. Imm. Meth., and Warren et al. (2011) Genome Res, 21:790) and may be employed in the course of practicing embodiments encompassed by the present invention. Similarly, methods for transfecting or transducing T cells with desired nucleic acids are well-known in the art (e.g., U.S. Pat. Publ. No. US 2004/0087025) as have adoptive transfer procedures using T cells of desired antigen-specificity (e.g., Schmitt et al. (2009) Hum. Gen, 20:1240, Dossett et al. (2009) Mol. Ther, 77:742, Till et al. (2008) Blood 772:2261, Wang et al. (2007) Hum. Gene Ther, 18:112, Kuball et al. (2007) Blood 709:2331, U.S. Pat. Publ, 2011/0243972. U.S. Pat. Publ, 2011/0189141, and Leen et al. (2007) Ann. Rev. Immunol, 25:243).

Any suitable immune cell may be modified to include a heterologous polynucleotide encompassed by the present invention, including, for example, a T cell, a NK cell, or a NK-T cell. In some embodiments, the cell may be a primary cell or a cell of a cell line. In some embodiments, a modified immune cell comprises a CD4⁺ T cell, a CD8⁺ T cell, or both. For purposes herein, the T cell may be any T cell, such as a cultured T cell, e.g., a primary T cell, or a T cell from a cultured T cell line, e.g., Jurkat, SupT1, etc., or a T cell obtained from a mammal. If obtained from a mammal, the T cell may be obtained from numerous sources, including but not limited to blood, bone marrow, lymph node, the thymus, or other tissues or fluids. T cells may also be enriched for or purified. In some embodiments, the T cell is a human T cell. In some embodiments, the T cell is a T cell isolated from a human. The T cell may be any type of T cell and may be of any developmental stage, including but not limited to, cytotoxic lymphocyte, cytotoxic lymphocyte precursor cell, cytotoxic lymphocyte progenitor cell, cytotoxic lymphocyte stem cell, CD4⁺/CD8⁺ double positive T cells, CD4⁺ helper T cells, e.g., Th1 and Th2 cells, CD4⁺ T cells, CD8⁺ T cells (e.g., cytotoxic T cells), tumor infiltrating lymphocytes (TILs), memory T cells (e.g., central memory T cells and effector memory T cells), naive T cells, and the like.

Any appropriate method may be used to transfect or transduce the cells, for example, T cells, or to administer the nucleotide sequences or compositions encompassed by methods described herein. Methods for delivering polynucleotides to host cells include, for example, use of cationic polymers, lipid-like molecules, and certain commercial products such as, for example, in vivo-jetPEI®. Other methods include ex vivo transduction, injection, electroporation. DEAE-dextran, sonication loading, liposome-mediated transfection, receptor-mediated transduction, microprojectile bombardment, transposon-mediated transfer, and the like. Still further methods of transfecting or transducing host cells employ vectors, described in further detail herein.

Modified immune cells as described herein may be functionally characterized using methodologies for assaying T cell activity, including determination of T cell binding, activation or induction and also including determination of T cell responses that are antigen-specific. Examples include determination of T cell proliferation, T cell cytokine release, antigen-specific T cell stimulation, MHC restricted T cell stimulation, CTL activity (e.g., by detecting ⁵¹Cr release from pre-loaded target cells), changes in T cell phenotypic marker expression, and other measures of T-cell functions.

Procedures for performing these and similar assays may be found, for example, in Lefkovits (Immunology Methods Manual: Hie Comprehensive Sourcebook of Techniques, 1998), as well as Current Protocols in Immunology, Weir, (1986) Handbook of Experimental Immunology, Blackwell Scientific, Boston, MA; Mishell and Shiigi (eds.) (1979) Selected Methods in Cellular Immunology, Freeman Publishing, San Francisco, CA; Green and Reed (1998) Science 281:1309, and references cited therein.

In some embodiments, apparent affinity for a binding protein, such as a TCR or antigen-binding portion thereof, may be measured by assessing binding to various concentrations of MHC multimers. “MHC-peptide multimer staining” refers to an assay used to detect antigen-specific T cells, which, in some embodiments, features a tetramer of MHC molecules, each comprising an identical peptide having an amino acid sequence that is cognate (e.g., identical or related to) at least one antigen (e.g., a PRAME immunogenic peptide), wherein the complex is capable of binding to a binding protein, such as a TCR or antigen-binding portion thereof, that recognizes the cognate antigen. Each of the MHC molecules may be tagged with a biotin molecule. Biotinylated MHC/peptides may be multimerized (e.g., tetramerized) by the addition of streptavidin, which may be fluorescently labeled.

The multimer may be detected by flow cytometry via the fluorescent label. In some embodiments, a pMHC multimer assay is used to detect or select enhanced affinity binding protein, such as a TCR or antigen-binding portion thereof, encompassed by the present invention. In some examples, apparent K_{D} of a binding protein, such as a TCR or antigen-binding portion thereof, is measured using 2-fold dilutions of labeled multimers at a range of concentrations, followed by determination of binding curves by non-linear regression, apparent K_{D} being determined as the concentration of ligand that yielded half-maximal binding.

Levels of cytokines may be determined using methods described herein, such as ELISA, ELISPOT, intracellular cytokine staining, and flow cytometry and combinations thereof (e.g., intracellular cytokine staining and flow cytometry).

Immune cell proliferation and clonal expansion resulting from an antigen-specific elicitation or stimulation of an immune response may be determined by isolating lymphocytes, such as circulating lymphocytes in samples of peripheral blood cells or cells from lymph nodes, stimulating the cells with antigen, and measuring cytokine production, cell proliferation and/or cell viability, such as by incorporation of tritiated thymidine or non-radioactive assays, such as MTT assays and the like. The effect of an immunogen described herein on the balance between a Th1 immune response and a Th2 immune response may be examined, for example, by determining levels of Th1 cytokines, such as IFN-g, IL-12. IL-2, and TNF-b, and Type 2 cytokines, such as IL-4, IL-5, IL-9, IL-10, and IL-13.

A host cell encompassed by the present invention may comprise a single polynucleotide that encodes a binding protein as described herein, or the binding protein may be encoded by more than one polynucleotide. In other words, components or portions of a binding protein may be encoded by two or more polynucleotides, which may be contained on a single nucleic acid molecule or may be contained on two or more nucleic acid molecules.

Moreover, as described further below and in the working examples, a host ell encompassed by the present invention may encode and/or express useful accessory proteins in addition to a binding protein as described herein, either on the same polynucleotide or a different polynucleotide as the binding protein or components thereof. For example, the host cell may encode and/or express CD8α, CD8β, a DN-TGFβR (e.g., a DN-TGFβRII), and/or a selectable protein marker, optionally wherein the selectable protein marker is DHFR.

In some embodiments, a polynucleotide encoding two or more components or portions of a binding protein encompassed by the present invention comprises the two or more coding sequences operatively associated in a single open reading frame. Such an arrangement can advantageously allow coordinated expression of desired gene products, such as, for example, contemporaneous expression of alpha- and beta-chains of a TCR, such that they are produced in about a 1:1 ratio. In some embodiments, two or more substituent gene products of a binding protein encompassed by the present invention, such as a TCR (e.g., alpha- and beta-chains) or CAR, are expressed as separate molecules and associate post-translationally. In further embodiments, two or more substituent gene products of a binding protein encompassed by the present invention are expressed as a single peptide with the parts separated by a cleavable or removable segment. For instance, self-cleaving peptides useful for expression of separable polypeptides encoded by a single polynucleotide or vector are known in the art and include, for example, a porcine teschovirus-1 2 A (P2A) peptide, a Thosea asigna virus 2A (T2A) peptide, an equine rhinitis A virus (ERAV) 2A (E2A) peptide, and a foot-and-mouth disease vims 2A (F2A) peptide.

In some embodiments, a binding protein encompassed by the present invention comprises one or more junction amino acids. “Junction amino acids” or “junction amino acid residues” refer to one or more (e.g., 2 to about 10) amino acid residues between two adjacent motifs, regions or domains of a polypeptide, such as between a binding domain and an adjacent constant domain or between a TCR chain and an adjacent self-cleaving peptide. Junction amino acids can result from the design of a construct that encodes a fusion protein (e.g., amino acid residues resulting from the use of a restriction enzyme site during the construction of a nucleic acid molecule encoding a fusion protein), or from cleavage of, for example, a self-cleaving peptide adjacent one or more domains of an encoded binding protein encompassed by the present invention (e.g., a P2A peptide disposed between a TCR α-chain and a TCR β-chain, the self-cleavage of which can leave one or more junction amino acids in the α-chain, the TCR β-chain, or both).

Engineered immune cells encompassed by the present invention may be administered as therapies for, e.g., a disorder characterized by PRAME expression (such as a non-malignant disorder, a hyperproliferative disorder, or a relapse of a hyperproliferative disorder characterized by PRAME expression). In some circumstances, it may be desirable to reduce or stop the activity associated with a cellular immunotherapy. Thus, in some embodiments, an engineered immune cell encompassed by the present invention comprises a heterologous polynucleotide encoding a binding protein and an accessory protein, such as a safety switch protein, which can be targeted using a cognate drug or other compound to selectively modulate the activity (e.g., lessen or ablate) of such cells when desirable. Safety switch proteins used in this regard include, for example, a truncated EGF receptor polypeptide (huEGFRt) that is devoid of extracellular N-terminal ligand binding domains and intracellular receptor tyrosine kinase activity but retains the native amino acid sequence, type I transmembrane cell surface localization, and a conformationally intact binding epitope for pharmaceutical-grade anti-EGFR monoclonal antibody, cetuximab (Erbitux) tEGF receptor (tEGFr; Wang et al. (2011) Blood 118:1255-1263), a caspase polypeptide (e.g., iCasp9; Straathof et al. (2005) Blood 105:4247-4254, Di Stasi et al. (2011) N. Engl. J. Med. 365:1673-1683, Zhou and Brenner (2016) Hematol, pii:S0301-472X:30513-30516), RQR8 (Philip et al. (2014) Blood 124:1277-1287), and a human c-myc protein tag (Kieback et al. (2008) Proc. Natl. Acad. Sci. USA 105:623-628)

Other accessory components useful for therapeutic cells comprise a tag or selection marker (e.g., a CD34 enrichment tag) that allows the cells to be identified, sorted, isolated, enriched, or tracked. For example, marked immune cells having desired characteristics (e.g., an antigen-specific TCR and a safety switch protein) may be sorted away from unmarked cells in a sample and more efficiently activated and expanded for inclusion in a therapeutic product of desired purity.

As used herein, the term “selection marker” comprises a nucleic acid construct that confers an identifiable change to a cell permitting detection and positive selection of immune cells transduced with a polynucleotide comprising a selection marker. For example, RQR is a selection marker that comprises a major extracellular loop of CD20 and two minimal CD34 binding sites. In some embodiments, an RQR-encoding polynucleotide comprises a polynucleotide that encodes the 16 amino acid CD34 minimal epitope. In some embodiments, such as certain embodiments provided in the examples herein, the CD34 minimal epitope is incorporated at the amino terminal position of the CD8 stalk domain (Q8). In further embodiments, the CD34 minimal binding site sequence may be combined with a target epitope for CD20 to form a compact marker/suicide gene for T cells (RQR8) (Philip et al, 2014). This construct allows for the selection of immune cells expressing the construct, with for example, CD34-specific antibody bound to magnetic beads (Miltenyi) and that utilizes clinically accepted pharmaceutical antibody, rituximab, that allows for the selective deletion of a transgene expressing engineered T cell (e.g., Philip et al. (2014) Blood 124:1277-1287, U.S. Pat. Publ, 2015-0093401, and U.S. Pat. Publ, 2018-0051089).

Further exemplary selection markers include several truncated type I transmembrane proteins normally not expressed on T cells: the truncated low-affinity nerve growth factor, truncated CD19, and truncated CD34 (e.g., Di Stasi et al. (2011) N. Engl. J. Med, 365:1673-1683, Mavilio et al. (1994) Blood 83:1988-1997, and Fehse et al. (2000) Mol. Ther, 7:448-456). A particularly attractive feature of CD19 and CD34 is the availability of the off-the-shelf Miltenyi CliniMACs™ selection system that can target these markers for clinical-grade sorting. However. CD19 and CD34 are relatively large surface proteins that may tax the vector packaging capacity and transcriptional efficiency of an integrating vector. Surface markers containing the extracellular, non-signaling domains or various proteins (e.g., CD19. CD34, LNGFR, etc.) also may be employed. Any selection marker may be employed and should be acceptable for good manufacturing practices. In some embodiments, selection markers are expressed with a polynucleotide that encodes a gene product of interest (e.g., a binding protein encompassed by the present invention, such as a TCR or CAR, or antigen-binding fragment thereof). Further examples of selection markers include, for example, reporters such as GFP, EGFP, β-gal or chloramphenicol acetyltransferase (CAT). In some embodiments, a selection marker, such as, for example, CD34 is expressed by a cell and the CD34 may be used to select enrich for, or isolate (e.g., by immunomagnetic selection) the transduced cells of interest for use in the methods described herein. As used herein, a CD34 marker is distinguished from an anti-CD34 antibody, or, for example, a scFv, TCR, or other antigen recognition moiety that binds to CD34.

In some embodiments, a selection marker comprises an RQR polypeptide, a truncated low-affinity nerve growth factor (tNGFR), a truncated CD19 (tCD19), a truncated CD34 (tCD34), or any combination thereof.

By way of background, inclusion of CD4⁺ T cells in an immunotherapy cell product can provide antigen-induced IL-2 secretion and augment persistence and function of transferred cytotoxic CD8⁺ T cells (e.g., Kennedy et al. (2008) Immunol. Rev, 222:129 and Nakanishi et al. Nature (2009) 52:510). In some embodiments, a class I-restricted TCR in CD4⁺ T cells may require the transfer of a CD8 co-receptor to enhance sensitivity of the TCR to class I HLA peptide complexes. CD4 co-receptors differ in structure to CD8 and cannot effectively substitute for CD8 co-receptors (e.g., Stone & Kranz (2013) Front. Immunol. 4:244 and Cole et al. (2012) Immunology 737:139). Thus, another accessory protein for use in the compositions and methods encompassed by the present invention comprises a CD8 co-receptor or component thereof. Engineered immune cells comprising a heterologous polynucleotide encoding a binding protein encompassed by the present invention may, in some embodiments, further comprise a heterologous polynucleotide encoding a CD8 co-receptor protein, or a beta-chain or alpha-chain component thereof.

A host cell may be efficiently transduced to contain, and may efficiently express, a single polynucleotide that encodes the binding protein, safety switch protein, selection marker, and CD8 co-receptor protein.

In one embodiment, the host cell encompassed by the present invention further includes a nucleic acid encoding a co-stimulatory molecule, such that the modified T cell expresses the co-stimulatory molecule. In some embodiments, the co-stimulatory domain is selected from CD3, CD27, CD28, CD83, CD86, CD127, 4-1BB, 4-1BBL, PD1 and PD1L.

In any of the foregoing embodiments, a host cell that express the binding protein described herein may be a universal immune cell. A “universal immune cell” comprises an immune cell that has been modified to reduce or eliminate expression of one or more endogenous genes that encode a polypeptide product selected from PD-1, LAG-3, CTLA4, TIM3, TIGIT, an HLA molecule, a TCR molecule, or any combination thereof. Without wishing to be bound by theory, certain endogenously expressed immune cell proteins may downregulate the immune activity of the modified immune cells (e.g., PD-1, LAG-3, CTLA4, TIGIT), or may interfere with the binding activity of a heterologously expressed binding protein encompassed by the present invention (e.g., an endogenous TCR that binds a non-PRAME antigen and interferes with the modified immune cell binding to a target cell that expresses a PRAME antigen such as a PRAME₄₂₅₋₄₃₃ immunogenic peptide comprising the amino acid sequence SLLQHLIGL in the context of a MHC molecule. Further, endogenous proteins (e.g., immune cell proteins, such as an HLA allele) expressed on a donor immune cell may be recognized as foreign by an allogeneic host, which may result in elimination or suppression of the modified donor immune cell by the allogeneic host.

Accordingly, decreasing or eliminating expression or activity of such endogenous genes or proteins can improve the activity, tolerance, or persistence of the modified immune cells in an autologous or allogenic host setting, and allows universal administration of the cells (e.g., to any recipient regardless of HLA type). In some embodiments, cells in accordance with the present invention are syngeneic, meaning that they are genetically identical or sufficiently identical and immunologically compatible as to allow for transplantation. In some embodiments, a universal immune cell is a donor cell (e.g., allogeneic) or an autologous cell. In some embodiments, a modified immune cell (e.g., a universal immune cell) encompassed by the present invention comprises a chromosomal gene knockout of one or more of a gene that encodes PD-1, LAG-3, CTLA4, TIM3, TIGIT, or other immune checkpoint, an HLA component (e.g., a gene that encodes an α1 macroglobulin, an α2 macroglobulin, an α3 macroglobulin, a β1 microglobulin, or a β2 microglobulin), or a TCR component (e.g., a gene that encodes a TCR variable region or a TCR constant region) (see, e.g., Torikai et al. (2016) Nature Sci. Rep, 6:21757; Torikai et al. (2012) Blood 179:5697; and Torikai et al. (2013) Blood 722:1341, which also provide representative, exemplary gene editing techniques, compositions, and adoptive cell therapies useful according to the present invention).

As used herein, the term “chromosomal gene knockout” refers to a genetic alteration or introduced inhibitory agent in a host cell that prevents (e.g., reduces, delays, suppresses, or abrogates) production, by the host cell, of a functionally active endogenous polypeptide product. Alterations resulting in a chromosomal gene knockout may include, for example, introduced nonsense mutations (including the formation of premature stop codons), missense mutations, gene deletion, and strand breaks, as well as the heterologous expression of inhibitory nucleic acid molecules that inhibit endogenous gene expression in the host cell.

In some embodiments, a chromosomal gene knock-out or gene knock-in may be made by chromosomal editing of a host cell. Chromosomal editing may be performed using, for example, endonucleases. As used herein “endonuclease” refers to an enzyme capable of catalyzing cleavage of a phosphodiester bond within a polynucleotide chain. In some embodiments, an endonuclease is capable of cleaving a targeted gene thereby inactivating or “knocking out” the targeted gene. An endonuclease may be a naturally occurring, recombinant, genetically modified, or fusion endonuclease. The nucleic acid strand breaks caused by the endonuclease are commonly repaired through the distinct mechanisms of homologous recombination or non-homologous end joining (NHEJ). During homologous recombination, a donor nucleic acid molecule may be used for a donor gene “knock-in”, for target gene “knock-out”, and optionally to inactivate a target gene through a donor gene knock in or target gene knock out event. NHEJ is an error-prone repair process that often results in changes to the DNA sequence at the site of the cleavage, e.g., a substitution, deletion, or addition of at least one nucleotide. NHEJ may be used to “knock-out” a target gene. Examples of endonucleases include zinc finger nucleases, TALE-nucleases. CRISPR-Cas nucleases, meganucleases, and megaTALs.

As used herein, a “zinc finger nuclease” (ZFN) refers to a fusion protein comprising a zinc finger DNA-binding domain fused to a non-specific DNA cleavage domain, such as a Fokl endonuclease. Each zinc finger motif of about 30 amino acids binds to about 3 base pairs of DNA, and amino acids at certain residues may be changed to alter triplet sequence specificity (e.g., Desjarlais et al. (1993) Proc. Natl. Acad. Sci, 90:2256-2260 and Wolfe et al. (1999) J. Mol. Biol, 255:1917-1934). Multiple zinc finger motifs may be linked in tandem to create binding specificity to desired DNA sequences, such as regions having a length ranging from about 9 to about 18 base pairs. By way of background. ZFNs mediate genome editing by catalyzing the formation of a site-specific DNA double strand break (DSB) in the genome, and targeted integration of a transgene comprising flanking sequences homologous to the genome at the site of DSB is facilitated by homology directed repair. Alternatively, a DSB generated by a ZFN can result in knock out of target gene via repair by non-homologous end joining (NHEJ), which is an error-prone cellular repair pathway that results in the insertion or deletion of nucleotides at the cleavage site. In some embodiments, a gene knockout comprises an insertion, a deletion, a mutation or a combination thereof, made using a ZFN molecule.

As used herein, a “transcription activator-like effector nuclease” (TALEN) refers to a fusion protein comprising a TALE DNA-binding domain and a DNA cleavage domain, such as a Fokl endonuclease. A “TALE DNA binding domain” or “TALE” is composed of one or more TALE repeat domains/units, each generally having a highly conserved 33-35 amino acid sequence with divergent 12th and 13th amino acids. The TALE repeat domains are involved in binding of the TALE to a target DNA sequence. The divergent amino acid residues, referred to as the repeat variable diresidue (RVD), correlate with specific nucleotide recognition. The natural (canonical) code for DNA recognition of these TALEs has been determined such that an HD (histine-aspartic acid) sequence at positions 12 and 13 of the TALE leads to the TALE binding to cytosine (C), NG (asparagine-glycine) binds to a T nucleotide, NI (asparagine-isoleucine) to A, NN (asparagine-asparagine) binds to a G or A nucleotide, and NG (asparagine-glycine) binds to a T nucleotide. Non-canonical (atypical) RVDs are also well-known in the art (e.g., U.S. Pat. Publ. No. US 2011/0301073, which atypical RVDs are incorporated by reference herein in their entirety). TALENs may be used to direct site-specific double-strand breaks (DSB) in the genome of T cells. Non-homologous end joining (NHEJ) ligates DNA from both sides of a double-strand break in which there is little or no sequence overlap for annealing, thereby introducing errors that knock out gene expression. Alternatively, homology directed repair can introduce a transgene at the site of DSB providing homologous flanking sequences are present in the transgene. In some embodiments, a gene knockout comprises an insertion, a deletion, a mutation or a combination thereof, and made using a TALEN molecule.

As used herein, a “clustered regularly interspaced short palindromic repeats/Cas” (CRISPR/Cas) nuclease system refers to a system that employs a CRISPR RNA (crRNA)-guided Cas nuclease to recognize target sites within a genome (known as protospacers) via base-pairing complementarity and then to cleave the DNA if a short, conserved protospacer associated motif (PAM) immediately follows 3′ of the complementary target sequence. CRISPR/Cas systems are classified into three types (i.e., type I, type II, and type III) based on the sequence and structure of the Cas nucleases. The crRNA-guided surveillance complexes in types I and III need multiple Cas subunits. Type 11 system, the most studied, comprises at least three components: an RNA-guided Cas9 nuclease, a crRNA, and a trans-acting crRNA (tracrRNA). The tracrRNA comprises a duplex forming region. A crRNA and a tracrRNA form a duplex that is capable of interacting with a Cas9 nuclease and guiding the Cas9/crRNA:tracrRNA complex to a specific site on the target DNA via Watson-Crick base-pairing between the spacer on the crRNA and the protospacer on the target DNA upstream from a PAM. Cas9 nuclease cleaves a double-stranded break within a region defined by the crRNA spacer. Repair by NHEJ results in insertions and/or deletions which disrupt expression of the targeted locus. Alternatively, a transgene with homologous flanking sequences may be introduced at the site of DSB via homology directed repair. The crRNA and tracrRNA may be engineered into a single guide RNA (sgRNA or gRNA) (e.g., Jinek et al. (2012) Science 337:816-821). Further, the region of the guide RNA complementary to the target site may be altered or programed to target a desired sequence (Xie et al. (2014) PLOS One 9:e100448, U.S. Pat. Publ. No. US 2014/0068797, U.S. Pat. Publ. No. US 2014/0186843, U.S. Pat. No. 8,697,359, and PCT Publ. No. WO 2015/071474). In some embodiments, a gene knockout comprises an insertion, a deletion, a mutation or a combination thereof, and made using a CRISPR/Cas nuclease system.

Exemplary gRNA sequences and methods of using the same to knock out endogenous genes that encode immune cell proteins include those described in Ren et al. (2017) Clin. Cancer Res, 23:2255-2266, which provides representative, exemplary gRNAs, CAS9 DNAs, vectors, and gene knockout techniques.

As used herein, a “meganuclease,” also referred to as a “homing endonuclease,” refers to an endodeoxyribonuclease characterized by a large recognition site (double stranded DNA sequences of about 12 to about 40 base pairs). Meganucleases may be divided into five families based on sequence and structure motifs: LAGLIDADG, GIY-YIG, HNH, His-Cys box, and PD-(D/E)XK. Exemplary meganucleases include i-Scel, I-Ceul, PI-PspI, RI-Sce, I-ScelV, I-Csml, I-PanI, I-Scell, I-Ppol, I-SceIII, I-Crel, I-Tevl, I-TevII and I-TevIII, whose recognition sequences are well-known (e.g., U.S. Pat. Nos. 5,420,032 and 6,833,252, Belfort et al. (1997) Nucl. Acids Res, 25:3379-3388, Dujon et al. (1989) Gene 52:115-118, Perler et al. (1994) Nucl. Acids Res, 22:1125-1127, Jasin (1996) Trends Genet, 72:224-228, Gimble et al. (1996) J. Mol. Biol, 263:163-180, and Argast et al. (1998) J. Mol. Biol, 280: 345-353).

In some embodiments, naturally-occurring meganucleases may be used to promote site-specific genome modification of a target of interest, such as an immune checkpoint, an HLA-encoding gene, or a TCR component-encoding gene.

In other embodiments, an engineered meganuclease having a novel binding specificity for a target gene is used for site-specific genome modification (see, e.g., Porteus et al. (2005) Nat. Biotechnol, 23:967-73, Sussman et al. (2004) J. Mol. Biol, 342:3141, Epinat et al. (2003) Nucl. Acids Res, 37:2952-2962, Chevalier et al. (2002) Mol. Cell 70:895-905, Ashworth et al. (2006) Nature 441:656-659, Paques et al. (2007) Curr. Gene Ther, 7:49-66, and U.S. Pat. Publ. Nos. US 2007/0117128. US 2006/0206949, US 2006/0153826, US 2006/0078552, and US 2004/0002092). In further embodiments, a chromosomal gene knockout is generated using a homing endonuclease that has been modified with modular DNA binding domains of TALENs to make a fusion protein known as a megaTAL MegaTALs may be utilized to not only knock-out one or more target genes, but to also introduce (knock in) heterologous or exogenous polynucleotides when used in combination with an exogenous donor template encoding a polypeptide of interest.

In some embodiments, a chromosomal gene knockout comprises an inhibitory nucleic acid molecule that is introduced into a host cell (e.g., an immune cell) comprising a heterologous polynucleotide encoding an antigen-specific receptor that binds (e.g., specifically and/or selectively) to a PRAME antigen, wherein the inhibitory nucleic acid molecule encodes a target-specific inhibitor and wherein the encoded target-specific inhibitor inhibits endogenous gene expression (i.e., of an immune checkpoint, an HLA component, or a TCR component, or any combination thereof) in the host immune cell.

A chromosomal gene knockout may be confirmed directly by DNA sequencing of the host immune cell following use of the knockout procedure or agent.

Chromosomal gene knockouts may also be inferred from the absence of gene expression (e.g., the absence of an mRNA or polypeptide product encoded by the gene) following the knockout.

In some embodiments, a host cell encompassed by the present invention is capable of specifically and/or selectively 50% or more of target cells that comprise a peptide-MHC (pMHC) complex comprising a PRAME immunogenic peptide in the context of an MHC molecule.

In some embodiments, the modified immune cell is capable of producing a cytokine when contacted with target cells that comprise a peptide-MHC (pMHC) complex comprising a PRAME immunogenic peptide in the context of an MHC molecule.

In some embodiments, the cytokine comprises IFN-γ or IL2. In some embodiments, the cytokine comprises TNF-α.

In some embodiments, the host cell is capable of producing a higher level of cytokine or a cytotoxic molecule when contacted with a target cell with expression of PRAME at a level of less than or equal to about 1,000 transcript per million transcripts (TPM), 950 TPM, 900 TPM, 850 TPM, 800 TPM, 750 TPM, 700 TPM, 650 TPM, 600 TPM, 550 TPM, 500 TPM, 450 TPM, 400 TPM, 350 TPM, 300 TPM, 250 TPM, 200 TPM, 150 TPM, 100 TPM, 95 TPM, 90 TPM, 85 TPM, 80 TPM, 75 TPM, 70 TPM, 65 TPM, 60 TPM, 55 TPM, 50 TPM, 45 TPM, 40 TPM, 35 TPM, 34 TPM, 33 TPM, 32 TPM, 31 TPM, 30 TPM, 29 TPM, 28 TPM, 27 TPM, 26 TPM, 25 TPM, 24 TPM, 23 TPM, 22 TPM, 21 TPM, 20 TPM, 19 TPM, 18 TPM, 17 TPM, 16 TPM, 15 TPM, 14 TPM, 13 TPM, 12 TPM, 11 TPM, 10 TPM, 9 TPM, 8 TPM, 7 TPM, 6 TPM, 5 TPM, 4 TPM, 3 TPM, 2 TPM, and 1 TPM, or any range in between, inclusive, such as less than or equal to about 1,000 TPM to less than or equal to about 35 TPM). In some embodiments, the low PRAME expression level is termed “heterozygous expression” meaning between about 1 TPM and about 35 TPM, or any range in between, inclusive, such as 1-32 TPM. For example, the host cell is capable of producing an at least 1.2 fold, 1.5 fold, 1.8 fold, 2.0 fold, 2.2 fold, 2.5 fold, 2.8 fold, 3 fold, 3.5 fold, 4 fold, 4.5 fold, 5 fold, 5.5 fold, 6 fold, 6.5 fold, 7 fold, 7.5 fold, 8 fold, 8.5 fold, 9 fold, 9.5 fold, 10 fold, 11 fold, 12 fold, 13 fold, 14 fold, 15 fold, 16 fold, 17 fold, 18 fold, 19 fold, 20 fold, 25 fold, 30 fold, 35 fold, 40 fold, 45 fold, 50 fold, 60 fold, 70 fold, 80 fold, 90 fold, 100 fold, 1000 fold, or more, or any range in between, inclusive, such as 1.2 fold to 2 fold, higher level of cytokine or a cytotoxic molecule.

In some embodiments, the host cell is capable of specifically and/or selectively killing a target cell expressing PRAME (e.g., a hyperproliferative cell expressing PRAME). In certain embodiments, the target cell expresses a PRAME immunogenic peptide in the context of an MHC molecule (e.g., a matched MHC molecule). In certain embodiments, the target cell expresses: (i) a polypeptide comprising or consisting of an amino acid sequence SLLQHLIGL; and (ii) a matched MHC molecule.

In some embodiments, host cells do not express PRAME antigen, are not recognized by a binding protein described herein, are not of serotype HLA-A*02, and/or do not express an HLA-A*02 allele, such as HLA-A*02:01, HLA-A*02:02, HLA-A*02:03, HLA-A*02:05, HLA-A*02:06, or HLA-A*02:07 allele. For example, a patient may receive host cells from a healthy donor who is PRAME-negative or HLA-A*02:01-negative, or even autologous cells that have selected and/or engineered. Cells, such as stem cells like hematopoietic stem cells, isolated from that donor (or engineered autologous cells) may be used as the source of transplant material. In parallel, T cells isolated from the same donor may be genetically engineered to recognize PRAME, such as by expressing a PRAME binding protein described herein. Donor cells, such as stem cells, may be used to engraft cell populations into the patient (e.g., hematopoietic stem cells used to reconstitute an immune system) and host cells may be infused into the patient with the goal of eliciting a highly specific anti-tumor effect. The engineered donor T cells may be designed to recognize and eliminate PRAME-expressing cells, such as all of the patient's native blood cells, including, for example, cancer cells like residual leukemia cells, which are PRAME-positive, thereby preventing relapse and promoting complete cures. Because the patient's new healthy blood cells are derived from the donor and are therefore either PRAME-negative, HLA-A*02 serotype negative, and/or or HLA-A*02 allele-negative, engineered cells described herein may have minimal toxic side effects. Such patient-matched host cells and treatment methods may be used according to therapeutic methods described further below.

In some embodiments, the killing is determined by a killing assay. In some embodiment, the killing assay is carrier out by coculturing the host cell and the target cell at a ratio from 20:1 to 0.625:1, for example, from 15:1 to 1.25:1, from 10:1 to 1.5:1, from 8:1 to 3:1, from 6:1 to 5:1, 20:1 to 5:1, 10:1 to 2.5:1 etc. In some embodiments, the target cell is pulsed with 1 μg/mL to 50 μg/mL of PRAME peptide, for example, from 1 μg/mL to 10 ng/mL, 500 ng/mL to 0.5 ng/mL, from 10 ng/mL to 10 μg/mL from 250 ng/mL to 1 ng/mL, from 50 ng/mL to 5 ng/mL, from 20 ng/mL to 10 ng/mL, etc.

In some embodiments, the host cell is capable of killing a higher number of target cells when contacted with target cells with a level of PRAME less than or equal to about 1,000 transcript per million transcripts (TPM), 950 TPM, 900 TPM, 850 TPM, 800 TPM, 750 TPM, 700 TPM, 650 TPM, 600 TPM, 550 TPM, 500 TPM, 450 TPM, 400 TPM, 350 TPM, 300 TPM, 250 TPM, 200 TPM, 150 TPM, 100 TPM, 95 TPM, 90 TPM, 85 TPM, 80 TPM, 75 TPM, 70 TPM, 65 TPM, 60 TPM, 55 TPM, 50 TPM, 45 TPM, 40 TPM, 35 TPM, 34 TPM, 33 TPM, 32 TPM, 31 TPM, 30 TPM, 29 TPM, 28 TPM, 27 TPM, 26 TPM, 25 TPM, 24 TPM, 23 TPM, 22 TPM, 21 TPM, 20 TPM, 19 TPM, 18 TPM, 17 TPM, 16 TPM, 15 TPM, 14 TPM, 13 TPM, 12 TPM, 11 TPM, 10 TPM, 9 TPM, 8 TPM, 7 TPM, 6 TPM, 5 TPM, 4 TPM, 3 TPM, 2 TPM, and 1 TPM, or any range in between, inclusive, such as less than or equal to about 1.000 TPM to less than or equal to about 35 TPM). In some embodiments, the low PRAME expression level is termed “heterozygous expression” meaning between about 1 TPM and about 35 TPM, or any range in between, inclusive, such as 1-32 TPM. For example, the host cell may be capable of killing an at least 1.2 fold, 1.5 fold, 1.8 fold, 2.0 fold, 2.2 fold, 2.5 fold, 2.8 fold, 3 fold, 3.5 fold, 4 fold, 4.5 fold, 5 fold, 5.5 fold, 6 fold, 6.5 fold, 7 fold, 7.5 fold, 8 fold, 8.5 fold, 9 fold, 9.5 fold, 10 fold, 11 fold, 12 fold, 13 fold, 14 fold, 15 fold, 16 fold, 17 fold, 18 fold, 19 fold, 20 fold, 25 fold, 30 fold, 35 fold, 40 fold, 45 fold, 50 fold, 60 fold, 70 fold, 80 fold, 90 fold, 100 fold, 1000 fold, or more, or any range in between, inclusive, such as 1.2 fold to 2 fold, higher number of target cells.

The present invention further provides a population of cells comprising at least one host cell described herein. The population of cells may be a heterogeneous population comprising the host cell comprising any of the recombinant expression vectors described, in addition to at least one other cell, e.g., a host cell (e.g., a T cell), which does not comprise any of the recombinant expression vectors, or a cell other than a T cell, e.g., a B cell, a macrophage, a neutrophil, an erythrocyte, a hepatocyte, an endothelial cell, an epithelial cells, a muscle cell, a brain cell, etc. Alternatively, the population of cells may be a substantially homogeneous population, in which the population comprises mainly of host cells (e.g., consisting essentially of) comprising the recombinant expression vector. The population also may be a clonal population of cells, in which all cells of the population are clones of a single host cell comprising a recombinant expression vector, such that all cells of the population comprise the recombinant expression vector. In one embodiment encompassed by the present invention, the population of cells is a clonal population comprising host cells comprising a recombinant expression vector as described herein.

In an embodiment encompassed by the present invention, the numbers of cells in the population may be rapidly expanded. Expansion of the numbers of T cells may be accomplished by any of a number of methods as are well-known in the art (e.g., U.S. Pat. Nos. 8,034,334 and 8,383,099, U.S. Pat. Publ. No. 2012/0244133, Dudley et al (2003) J. Immunother, 26:332-242, and Riddell et al. (1990) J. Immunol. Methods 128:189-201). For example, expansion of the numbers of T cells may be carried out by culturing the T cells with OKT3 antibody, IL-2, and feeder PBMC (e.g., irradiated allogeneic PBMC).

### V. Pharmaceutical Compositions

In another aspect encompassed by the present invention, pharmaceutical compositions are provided herein comprising compositions described herein (e.g., binding proteins, nucleic acids, cells, and the like) and a pharmaceutically acceptable carrier, diluent, or excipient.

The term “pharmaceutically acceptable” refers to those agents, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Agents and other compositions encompassed by the present invention may be specially formulated for administration in solid or liquid form, including those adapted for various routes of administration, such as (1) oral administration, for example, drenches (aqueous or non-aqueous solutions or suspensions), tablets, boluses, powders, granules, pastes; (2) parenteral administration, for example, by subcutaneous, intramuscular or intravenous injection as, for example, a sterile solution or suspension; (3) topical application, for example, as a cream, ointment or spray applied to the skin; (4) intravaginally or intrarectally, for example, as a pessary, cream or foam; or (5) aerosol, for example, as an aqueous aerosol, liposomal preparation or solid particles containing the compound. Any appropriate form factor for an agent or composition described herein, such as, but not limited to, tablets, capsules, liquid syrups, soft gels, suppositories, and enemas, is contemplated.

Pharmaceutical compositions encompassed by the present invention may be presented as discrete dosage forms, such as capsules, sachets, or tablets, or liquids or aerosol sprays each containing a pre-determined amount of an active ingredient as a powder or in granules, a solution, or a suspension in an aqueous or non-aqueous liquid, an oil-in-water emulsion, a water-in-oil liquid emulsion, powders for reconstitution, powders for oral consumptions, bottles (including powders or liquids in a bottle), orally dissolving films, lozenges, pastes, tubes, gums, and packs. Such dosage forms may be prepared by any of the methods of pharmacy.

Suitable excipients include water, saline, dextrose, glycerol, or the like and combinations thereof. In some embodiments, compositions comprising host cells, binding proteins, or fusion proteins as disclosed herein further comprise a suitable infusion media. Suitable infusion media may be any isotonic medium formulation, typically normal saline, Normosol™-R (Abbott) or Plasma-Lyte™ A (Baxter), 5% dextrose in water, Ringer's lactate may be utilized. An infusion medium may be supplemented with human serum albumin or other human serum components. Unit doses comprising an effective amount of a host cell, or composition are also contemplated.

Also provided herein are unit doses that comprise an effective amount of a host cell or of a composition comprising the host cell. As described herein, host cells include immune cells, T cells (CD4⁺ T cells and/or CD8+ T cells), cytotoxic lymphocytes (e.g., cytotoxic T cells and/or natural killer (NK) cells), and the like. For example, in some embodiments, a unit dose comprises a composition comprising at least about 30%, at least about 40%, at least about 50%, at least about 60%), at least about 70%, at least about 80%, at least about 85%, at least about 90%, or at least about 95% engineered cells, either alone or in combination with other cells, such as comprising at least about 30%, at least about 40%, at least about 50%, at least about 60%), at least about 70%, at least about 80%, at least about 85%, at least about 90%, or at least about 95% other cells. In some embodiments, undesired cells are present at a reduced amount or substantially not present, such as less than about 50%, less than about 40%, less than about 30%, less than about 20%, less than about 10%, less than about 5%, or less then about 1% the population of cells in the composition.

The amount of cells in a composition or unit dose is at least one cell (for example, at least one engineered CD8⁺ T cell, engineered CD4⁺ T cell, and/or NK cell) or is more typically greater than 10² cells, for example, up to 10⁶, up to 10⁷, up to 10⁸ cells, up to 10⁹ cells, or more than 10¹⁰ cells. In some embodiments, the cells are administered in a range from about 106 to about 10¹⁰ cells/m², such as in a range of about 10⁵ to about 10⁹ cells/m². The number of cells will depend upon the ultimate use for which the composition is intended as well the type of cells included therein. For example, cells modified to contain a binding protein specific for a particular antigen will comprise a cell population containing at least 30%, 35%, 40%, 45%, 50%, 55%, 60%, 65%, 70%, 75%, 80%, 85%, 90%, 95% or more of such cells. For uses provided herein, cells are generally in a volume of a liter or less, 500 ml or less, 250 ml or less, or 100 ml or less. In embodiments, the density of the desired cells is typically greater than 10⁴ cells/ml and generally is greater than 10⁷ cells/ml, generally 10⁸ cells/ml or greater. The cells may be administered as a single infusion or in multiple infusions over a range of time. A clinically relevant number of immune cells may be apportioned into multiple infusions that cumulatively equal or exceed 10⁶, 10⁷, 10⁸, 10⁹, 10¹⁰, or 10¹¹ cells. In some embodiments, a unit dose of the engineered immune cells may be co-administered with (e.g., simultaneously or contemporaneously) hematopoietic stem cells from an allogeneic donor.

Pharmaceutical compositions may be administered in a manner appropriate to the disease or condition to be treated (or prevented) as determined by persons skilled in the medical art. An appropriate dose and a suitable duration and frequency of administration of the compositions will be determined by such factors as the health condition of the patient, size of the patient (i.e., weight, mass, or body area), the type and severity of the patient's condition, the particular form of the active ingredient, and the method of administration. In general, an appropriate dose and treatment regimen provide the composition(s) in an amount sufficient to provide therapeutic and/or prophylactic benefit (such as described herein, including an improved clinical outcome, such as more frequent complete or partial remissions, or longer disease-free and/or overall survival, or a lessening of symptom severity).

An effective amount of a pharmaceutical composition refers to an amount sufficient, at dosages and for periods of time needed, to achieve the desired clinical results or beneficial treatment, as described herein. An effective amount may be delivered in one or more administrations. If the administration is to a subject already known or confirmed to have a disease or disease-state, the term “therapeutically effective amount” may be used in reference to treatment, whereas “prophylactically effective amount” may be used to describe administrating an effective amount to a subject that is susceptible or at risk of developing a disease or disease-state (e.g., recurrence) as a preventative course.

The pharmaceutical compositions described herein may be presented in unit-dose or multi-dose containers, such as sealed ampoules or vials. Such containers may be frozen to preserve the stability of the formulation until infusion into the patient. In some embodiments, a unit dose comprises a host cell as described herein at a dose of about 10⁷ cells/m² to about 10¹¹ cells/m². The development of suitable dosing and treatment regimens for using the particular compositions described herein in a variety of treatment regimens, including e.g., parenteral or intravenous administration or formulation.

If the subject composition is administered parenterally, the composition may also include sterile aqueous or oleaginous solution or suspension. Suitable non-toxic parenterally acceptable diluents or solvents include water, Ringer's solution, isotonic salt solution, 1,3-butanediol, ethanol, propylene glycol or polyethylene glycols in mixtures with water. Aqueous solutions or suspensions may further comprise one or more buffering agents, such as sodium acetate, sodium citrate, sodium borate or sodium tartrate. Of course, any material used in preparing any dosage unit formulation should be pharmaceutically pure and substantially non-toxic in the amounts employed. In addition, the active compounds may be incorporated into sustained-release preparation and formulations. Dosage unit form, as used herein, refers to physically discrete units suited as unitary dosages for the subject to be treated; each unit may contain a predetermined quantity of engineered immune cells or active compound calculated to produce the desired effect in association with an appropriate pharmaceutical carrier.

In some embodiments, the pharmaceutical composition described herein and as described above for immunogenic compositions representatively exemplified for peptides, when administered to a subject, can elicit an immune response against a cell of interest that expresses PRAME. Such pharmaceutical compositions may be useful as vaccines for prophylactic and/or therapeutic treatment of a disorder characterized by PRAME expression (e.g., a non-malignant disorder, a hyperproliferative disorder, or a relapse of a hyperproliferative disorder characterized by PRAME expression).

In some embodiments, the pharmaceutical composition further comprises a physiologically acceptable adjuvant. In some embodiments, the adjuvant employed provides for increased immunogenicity of the pharmaceutical composition. Such a further immune response stimulating compound or adjuvant may be (i) admixed to the pharmaceutical composition in accordance with the present invention after reconstitution of the peptides and optional emulsification with an oil-based adjuvant as defined above. (ii) may be part of the reconstitution composition encompassed by the present invention defined above, (iii) may be physically linked to the peptide(s) to be reconstituted or (iv) may be administered separately to the subject, mammal or human, to be treated. The adjuvant may be one that provides for slow release of antigen (e.g., the adjuvant may be a liposome), or it may be an adjuvant that is immunogenic in its own right thereby functioning synergistically with antigens. For example, the adjuvant may be a known adjuvant or other substance that promotes antigen uptake, recruits immune system cells to the site of administration, or facilitates the immune activation of responding lymphoid cells. Adjuvants include, but are not limited to, immunomodulatory molecules (e.g., cytokines), oil and water emulsions, aluminum hydroxide, glucan, dextran sulfate, iron oxide, sodium alginate, bacto-adjuvant, synthetic polymers such as poly amino acids and co-polymers of amino acids, saponin, paraffin oil, and muramyl dipeptide. In some embodiments, the adjuvant is adjuvant 65, α-GalCer, aluminum phosphate, aluminum hydroxide, calcium phosphate, β-glucan peptide. CpG DNA, GM-CSF, GPI-0100, IFA, IFN-γ, IL-17, lipid A, lipopolysaccharide, Lipovant, Montanide™, N-acetyl-muramyl-L-alanyl-D-isoglutamine, pam3CSK4, quil A, trehalose dimycolate, or zymosan.

In some embodiments, the adjuvant is an immunomodulatory molecule. For example, the immunomodulatory molecule may be a recombinant protein cytokine, chemokine, or immunostimulatory agent or nucleic acid encoding cytokines, chemokines, or immunostimulatory agents designed to enhance the immunologic response.

Examples of immunomodulatory cytokines include interferons (e.g., IFNα, IFNβ and IFNγ), interleukins (e.g., IL-1, IL-2, IL-3, IL-4, IL-5, IL-6, IL-7, IL-8, IL-9, IL-10, IL-12. IL-17 and IL-20), tumor necrosis factors (e.g., TNFα and TNFβ), erythropoietin (EPO), FLT-3 ligand, gIp10, TCA-3, MCP-1, MIF, MIP-1.alpha., MIP-1β, Rantes, macrophage colony stimulating factor (M-CSF), granulocyte colony stimulating factor (G-CSF), and granulocyte-macrophage colony stimulating factor (GM-CSF), as well as functional fragments of any of the foregoing.

In some embodiments, an immunomodulatory chemokine that binds to a chemokine receptor, i.e., a CXC, CC. C, or CX3C chemokine receptor, also may be included in the compositions provided here. Examples of chemokines include, but are not limited to, Mip1α, Mip-1β, Mip-3α (Larc), Mip-3β, Rantes, Hcc-1, Mpif-1, Mpif-2, Mcp-1, Mcp-2, Mcp-3, Mcp-4, Mcp-5, Eotaxin, Tarc, Elc, I309, IL-8, Gep-2 Gro-α, Gro-β, Gro-γ, Nap-2, Ena-78, Gcp-2, Ip-10, Mig, I-Tac, Sdf-1, and Bca-1 (Blc), as well as functional fragments of any of the foregoing.

In some embodiments, the composition comprises a binding protein (e.g., a TCR, an antigen-binding fragment of a TCR, a CAR, or a fusion protein comprising a TCR and an effector domain), a TCRα and/or TCRβ polypeptide described herein. In some embodiments, the composition comprises a nucleic acid encoding a binding protein, a TCRα and/or TCRβ polypeptide described herein, such as a DNA molecule encoding a binding protein, a TCRα and/or TCRβ polypeptide. In some embodiments, the composition comprises an expression vector comprising an open reading frame encoding a binding protein, a TCRα and/or TCRβ polypeptide.

When taken up by a cell (e.g., T cells, NK cells, etc.), a DNA molecule may be present in the cell as an extrachromosomal molecule and/or may integrate into the chromosome. DNA may be introduced into cells in the form of a plasmid which may remain as separate genetic material. Alternatively, linear DNAs that may integrate into the chromosome may be introduced into the cell. Optionally, when introducing DNA into a cell, reagents which promote DNA integration into chromosomes may be added.

### VI. Uses and Methods

The compositions described herein may be used in a variety of diagnostic, prognostic, and therapeutic applications. In any method described herein, such as a diagnostic method, prognostic method, therapeutic method, or combination thereof, all steps of the method can be performed by a single actor or, alternatively, by more than one actor. For example, diagnosis can be performed directly by the actor providing therapeutic treatment. Alternatively, a person providing a therapeutic agent can request that a diagnostic assay be performed. The diagnostician and/or the therapeutic interventionist can interpret the diagnostic assay results to determine a therapeutic strategy. Similarly, such alternative processes can apply to other assays, such as prognostic assays.

In some uses and methods encompassed by the present invention, subjects or subject samples are utilized. In some embodiments, the subject is an animal. The animal may be of either sex and may be at any stage of development. In some embodiments, the animals is a vertebrate, such as a mammal. In some embodiments, the subject is a non-human mammal. In some embodiments, the subject is a domesticated animal, such as a dog, cat, cow, pig, horse, sheep, or goat. In some embodiments, the subject is a companion animal, such as a dog or cat. In some embodiments, the subject is a livestock animal, such as a cow, pig, horse, sheep, or goat. In some embodiments, the subject is a zoo animal. In some embodiments, the subject is a research animal, such as a rodent (e.g., mouse or rat), dog, pig, or non-human primate. In some embodiments, the animal is a genetically engineered animal. In some embodiments, the animal is a transgenic animal (e.g., transgenic mice and transgenic pigs). In some embodiments, the subject is a fish or reptile.

In some embodiments, the subject is a rodent, such as a mouse. In some such embodiments, the mouse is a transgenic mouse, such as a mouse expressing human MHC (i.e., HLA) molecules (e.g., Nicholson et a (2012) Adv. Hematol, 2012:404081). In some embodiments, the subject is a transgenic mouse expressing human TCRs or is an antigen-negative mouse (e.g., Li et al. (2010) Nat. Med, 16:1029-1034 and Obenaus et al. (2015) Nat. Biotechnol, 33:402-407). In some embodiments, the subject is a transgenic mouse expressing human HLA molecules and human TCRs. In some embodiments, such as where the subject is a transgenic HLA mouse, the identified TCRs are modified, e.g., to be chimeric or humanized. In some embodiments, the TCR scaffold is modified, such as analogous to known binding protein humanizing methods.

In some embodiments, the subject is a human. In some embodiments, the subject is an animal model of a disorder characterized by PRAME expression (e.g., a non-malignant disorder, the hyperproliferative disorder, or the relapse of a hyperproliferative disorder characterized by expression of a PRAME antigen). For example, the animal model may be an orthotopic xenograft animal model of a human-derived cancer.

In some embodiments, the subject is a human, such as a human with a disorder characterized by PRAME expression.

The methods described herein may be used to treat a subject in need thereof. As used herein, a “subject in need thereof” includes any subject who has a disorder characterized by PRAME expression, a relapse of a disorder characterized by PRAME expression, and/or who is predisposed to a disorder characterized by PRAME expression. As described herein, a disorder characterized by PRAME expression may be a non-malignant disorder, a hyperproliferative disorder, or a relapse of a hyperproliferative disorder characterized by PRAME expression.

In some embodiments of the methods encompassed by the present invention, the subject has not undergone treatment for a disorder characterized by PRAME expression, such as chemotherapy, radiation therapy, targeted therapy, and/or immunotherapies. In some embodiments, the subject has undergone treatment for a disorder characterized by PRAME expression, such as chemotherapy, radiation therapy, targeted therapy, and/or immunotherapies.

In some embodiments, the subject has had surgery to remove cancerous or precancerous tissue. In some embodiments, the cancerous tissue has not been removed, e.g., the cancerous tissue may be located in an inoperable region of the body, such as in a tissue that is essential for life, or in a region where a surgical procedure would cause considerable risk of harm to the patient.

In some embodiments, the subject or cells thereof are resistant to a therapy of relevance, such as resistant to standard of care therapy, immune checkpoint inhibitor therapy, and the like. For example, modulating one or more biomarkers encompassed by the present invention may overcome resistance to immune checkpoint inhibitor therapy.

In some embodiments, the subjects are in need of modulation according to compositions and methods described herein, such as having been identified as having an unwanted absence, presence, or aberrant PRAME expression.

a. Diagnostic Methods

In an aspect encompassed by the present invention, provided herein are diagnostic methods for detecting the presence or absence of a PRAME antigen and/or a cell of interest expressing PRAME, comprising detecting the presence or absence of said PRAME antigen in a sample by use of at least one binding protein, or at least one host cell described herein. In some embodiments, the method further comprising obtaining the sample (e.g., from a subject). In some embodiments, the at least one binding protein or the at least one host cell, forms a complex with a PRAME peptide epitope in the context of an MHC molecule, and the complex is detected in the form of fluorescence activated cell sorting (FACS), enzyme linked immunosorbent assay (ELISA), radioimmune assay (RIA), immunochemically, Western blot, or intracellular flow assay.

In an aspect encompassed by the present invention, provided herein are diagnostic methods for detecting the level of a disorder characterized by PRAME expression, such as a non-malignant disorder, a hyperproliferative disorder, or a relapse of a hyperproliferative disorder characterized by PRAME expression in a subject, comprising: a) contacting a sample obtained from the subject with at least one agent (e.g., a PRAME immunogenic peptide, PRAME immunogenic peptide-MHC complex (pMHC), binding protein, at least one host cell, or a population of host cells described herein; and b) detecting the level of reactivity, wherein a higher level of reactivity compared to a control level indicates that the level of a disorder characterized by PRAME expression (e.g., a non-malignant disorder, a hyperproliferative disorder, or a relapse of a hyperproliferative disorder characterized by PRAME expression) in the subject.

In some embodiments, the level of reactivity is indicated by T cell activation or effector function, such as, but not limited to, T cell proliferation, killing, or cytokine release. The control level may be a reference number or a level of a healthy subject who has no exposure to a disorder characterized by PRAME expression, e.g., a non-alignment disorder, a hyperproliferative disorder, or a relapse of a hyperproliferative disorder characterized by PRAME expression.

A biological sample may be obtained from a subject for determining the presence and level of an immune response to a peptide antigen (e.g., a PRAME antigen) as described herein. A “biological sample” as used herein may be a blood sample (from which serum or plasma may be prepared), biopsy specimen, body fluids (e.g., blood, isolated PBMCs, isolated T cells, lung lavage, ascites, mucosal washings, synovial fluid, etc.), bone marrow, lymph nodes, tissue explant, organ culture, or any other tissue or cell preparation from the subject or a biological source. Biological samples may also be obtained from the subject prior to receiving any pharmaceutical composition, which biological sample is useful as a control for establishing baseline data.

Antigen-specific T cell responses are typically determined by comparisons of observed T cell responses according to any of the herein described T cell functional parameters (e.g., proliferation, cytokine release, CTL activity, altered cell surface marker phenotype, etc.) that may be made between T cells that are exposed to a cognate antigen in an appropriate context (e.g., the antigen used to prime or activate the T cells, when presented by immunocompatible antigen-presenting cells) and T cells from the same source population that are exposed instead to a structurally distinct or irrelevant control antigen. A response to the cognate antigen that is greater, with statistical significance, than the response to the control antigen signifies antigen-specificity.

The level of an immune response, such as a cytotoxic T lymphocyte (CTL) immune response may be determined by any one of numerous immunological methods described herein and routinely practiced in the art. For example, the level of a CTL immune response may be determined prior to and following administration of any one of the herein described binding proteins expressed by, for example, a T cell. Cytotoxicity assays for determining CTL activity may be performed using any one of several techniques and methods routinely practiced in the art (e.g., Henkart et al., “Cytotoxic T-Lymphocytes” in Fundamental Immunology, Paul (ed.) (2003 Lippincott Williams & Wilkins, Philadelphia, PA), pages 1127-50, and references cited therein).

The present invention provides, in part, methods, systems, and code for accurately classifying whether a biological sample is associated with an output of interest, such as expression of a target of interest, such as PRAME. In some embodiments, the present invention is useful for classifying a sample (e.g., from a subject) as associated with or at risk for responding to or not responding to therapy for a disorder characterized by PRAME expression using a statistical algorithm and/or empirical data.

An exemplary method for detecting the amount or activity of PRAME, and thus useful for classifying whether a sample is likely or unlikely to respond to a therapy for a disorder characterized by PRAME expression involves contacting a biological sample with an agent, such as a PRAME immunogenic peptide or binding agent described herein, capable of detecting the amount or activity of PRAME in the biological sample. In some embodiments, the method further comprises obtaining a biological sample, such as from a test subject. In some embodiments, at least one agent is used, wherein two, three, four, five, six, seven, eight, nine, ten, or more such agents may be used in combination (e.g., in sandwich ELISAs) or in serial. In certain instances, the statistical algorithm is a single learning statistical classifier system. For example, a single learning statistical classifier system may be used to classify a sample as a based upon a prediction or probability value and the presence or level of the biomarker. The use of a single learning statistical classifier system typically classifies the sample with a sensitivity, specificity, positive predictive value, negative predictive value, and/or overall accuracy of at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%.

Other suitable statistical algorithms are well-known to those of skill in the art. For example, learning statistical classifier systems include a machine learning algorithmic technique capable of adapting to complex data sets (e.g., panel of markers of interest) and making decisions based upon such data sets. In some embodiments, a single learning statistical classifier system such as a classification tree (e.g., random forest) is used. In other embodiments, a combination of 2, 3, 4, 5, 6, 7, 8, 9, 10, or more learning statistical classifier systems are used, preferably in tandem. Examples of learning statistical classifier systems include, but are not limited to, those using inductive learning (e.g., decision/classification trees such as random forests, classification and regression trees (C & RT), boosted trees, etc.), Probably Approximately Correct (PAC) learning, connectionist learning (e.g., neural networks (NN), artificial neural networks (ANN), neuro fuzzy networks (NFN), network structures, perceptrons such as multi-layer perceptrons, multi-layer feed-forward networks, applications of neural networks, Bayesian learning in belief networks, etc.), reinforcement learning (e.g., passive learning in a known environment such as naive learning, adaptive dynamic learning, and temporal difference learning, passive learning in an unknown environment, active learning in an unknown environment, learning action-value functions, applications of reinforcement learning, etc.), and genetic algorithms and evolutionary programming. Other learning statistical classifier systems include support vector machines (e.g., Kernel methods), multivariate adaptive regression splines (MARS), Levenberg-Marquardt algorithms, Gauss-Newton algorithms, mixtures of Gaussians, gradient descent algorithms, and learning vector quantization (LVQ). In certain embodiments, the method encompassed by the present invention further comprises sending the sample classification results to a clinician, e.g., an oncologist.

In some embodiments, the diagnosis of a subject (e.g., including HLA typing and/or loss of heterozygosity (LOH) to determine compatibility with TCR-HLA complex binding by TCRs of interest) is followed by administering to the individual a therapeutically effective amount of a defined treatment based upon the diagnosis.

In some embodiments, the methods further involve obtaining a control biological sample (e.g., biological sample from a subject who does not have a disorder characterized by PRAME expression, a subject who is in remission, a subject whose disorder is susceptible to therapy, a subject whose disorder is progressing, or other subjects of interest).

In some embodiments of analytical methods described herein, PRAME expression (e.g., in a sample from a subject) is compared to a pre-determined control (standard) sample. The sample from the subject is typically from a diseased tissue, such as cancer cells or tissues. The control sample may be from the same subject or from a different subject. The control sample is typically a normal, non-diseased sample. However, in some embodiments, such as for staging of disease or for evaluating the efficacy of treatment, the control sample may be from a diseased tissue. The control sample may be a combination of samples from several different subjects. In some embodiments, the PRAME expression measurement(s) from a subject is compared to a pre-determined level. This pre-determined level is typically obtained from normal samples. As described herein, a “pre-determined” expression may be used to, by way of example only, evaluate a subject that may be selected for treatment, evaluate a response to cancer, and/or evaluate a response to a combination cancer therapy. A pre-determined biomarker amount and/or activity measurement(s) may be determined in populations of patients with or without a disorder characterized by PRAME expression. The pre-determined biomarker amount and/or activity measurement(s) may be a single number, equally applicable to every patient, or the pre-determined biomarker amount and/or activity measurement(s) may vary according to specific sub-populations of patients. Age, weight, height, and other factors of a subject may affect the pre-determined biomarker amount and/or activity measurement(s) of the individual. Furthermore, the pre-determined biomarker amount and/or activity may be determined for each subject individually. In one embodiment, the amounts determined and/or compared in a method described herein are based on absolute measurements.

In another embodiment, the amounts determined and/or compared in a method described herein are based on relative measurements, such as ratios (e.g., biomarker copy numbers, level, and/or activity before a treatment vs. after a treatment, such biomarker measurements relative to a spiked or man-made control, such biomarker measurements relative to the expression of a housekeeping gene, and the like). For example, the relative analysis may be based on the ratio of pre-treatment biomarker measurement as compared to post-treatment biomarker measurement. Pre-treatment biomarker measurement may be made at any time prior to initiation of a therapy. Post-treatment biomarker measurement may be made at any time after initiation of therapy. In some embodiments, post-treatment biomarker measurements are made 1, 2, 3, 4, 5, 6, 7, 8, 9, 10, 11, 12, 13, 14, 15, 16, 17, 18, 19, 20 weeks or more after initiation of therapy, and even longer toward indefinitely for continued monitoring. Treatment may comprise therapy to treat the disorder characterized by PRAME expression, either alone or in combination with other agents, such as anti-cancer agents like chemotherapy or immune checkpoint inhibitors.

The pre-determined PRAME expression may be any suitable standard. For example, the pre-determined PRAME expression may be obtained from the same or a different subject for whom a subject selection is being assessed. In one embodiment, the pre-determined biomarker amount and/or activity measurement(s) may be obtained from a previous assessment of the same patient. In such a manner, the progress of the selection of the patient may be monitored over time. In addition, the control may be obtained from an assessment of another human or multiple humans, e.g., selected groups of humans, if the subject is a human. In such a manner, the extent of the selection of the human for whom selection is being assessed may be compared to suitable other humans, e.g., other humans who are in a similar situation to the human of interest, such as those suffering from similar or the same condition(s) and/or of the same ethnic group.

In some embodiments, the change of PRAME expression from the pre-determined level is about 0.1, 0.2, 0.3, 0.4, 0.5, 0.6, 0.7, 0.8, 0.9, 1.0, 1.5, 2.0, 2.5, 3.0, 3.5, 4.0, 4.5, or 5.0 fold or greater, or any range in between, inclusive. Such cut-off values apply equally when the measurement is based on relative changes, such as based on the ratio of pre-treatment biomarker measurement as compared to post-treatment biomarker measurement.

In some embodiments, PRAME expression may be detected and/or quantified by detecting or quantifying PRAME polypeptide or antigen thereof, such as by using a composition described herein. The polypeptide may be detected and quantified by any of a number of means well-known to those of skill in the art, such as by immunodiffusion, immunoelectrophoresis, radioimmunoassay (RIA), enzyme-linked immunosorbent assays (ELISAs), immunofluorescent assays, Western blotting, binder-ligand assays, immunohistochemical techniques, agglutination, complement assays, high performance liquid chromatography (HPLC), thin layer chromatography (TLC), hyperdiffusion chromatography, and the like (e.g., Basic and Clinical Immunology, Sites and Terr, eds., Appleton and Lange, Norwalk, Conn. pp 217-262, 1991).

b. Therapeutic Methods

In an aspect encompassed by the present invention, provided herein are methods for preventing and/or treating a disorder characterized by PRAME expression, e.g., a non-malignant disorder, a hyperproliferative disorder, or a relapse of a hyperproliferative disorder characterized by PRAME expression, and/or for inducing an immune response against a cell of interest, such as a hyperproliferative cell, expressing PRAME. In some embodiments, the method comprises administering to a subject a therapeutically effective amount of a composition described herein, such as an immunogenic composition, such as a composition comprising cells expressing at least one binding protein described herein. The methods encompassed by the present invention also may be used to determine the responsiveness to therapy for many different disorders characterized by PRAME expression in subjects, such as those described herein.

In some embodiments, the disorder characterized by PRAME expression is a cancer. The terms “cancer” or “tumor” or “hyperproliferative” refer to the presence of cells possessing characteristics typical of cancer-causing cells, such as uncontrolled proliferation, immortality, invasive or metastatic potential, rapid growth, and certain characteristic morphological features. In some embodiments, such cells exhibit such characteristics in part or in full due to the expression and activity of immune checkpoint proteins, such as PD-1, PD-L1, PD-L2, and/or CTLA-4.

Cancer cells are often in the form of a tumor, but such cells may exist alone within an animal, or may be a non-tumorigenic cancer cell, such as in a hematologic cancer like leukemia. As used herein, the term “cancer” includes premalignant as well as malignant cancers. Cancers include, but are not limited to, a variety of cancers, carcinoma including that of the bladder (including accelerated and metastatic bladder cancer), breast, colon (including colorectal cancer), kidney, liver, lung (including small and non-small cell lung cancer and lung adenocarcinoma), ovary, prostate, testes, genitourinary tract, lymphatic system, rectum, larynx, pancreas (including exocrine pancreatic carcinoma), esophagus, stomach, gall bladder, cervix, thyroid, and skin (including squamous cell carcinoma); hematopoietic tumors of lymphoid lineage including leukemia, acute lymphocytic leukemia, acute lymphoblastic leukemia, B-cell lymphoma, T-cell lymphoma, Hodgkins lymphoma, non-Hodgkins lymphoma, hairy cell lymphoma, histiocytic lymphoma, and Burketts lymphoma; hematopoietic tumors of myeloid lineage including acute and chronic myelogenous leukemias, myelodysplastic syndrome, myeloid leukemia, and promyelocytic leukemia; tumors of the central and peripheral nervous system including astrocytoma, neuroblastoma, glioma, and schwannomas; tumors of mesenchymal origin including fibrosarcoma, rhabdomyosarcoma, and osteosarcoma; other tumors including melanoma, xenoderoma pigmentosum, keratoacanthoma, seminoma, thyroid follicular cancer, and teratocarcinoma; melanoma, unresectable stage III or IV malignant melanoma, squamous cell carcinoma, small-cell lung cancer, non-small cell lung cancer, glioma, gastrointestinal cancer, renal cancer, ovarian cancer, liver cancer, colorectal cancer, endometrial cancer, kidney cancer, prostate cancer, thyroid cancer, neuroblastoma, pancreatic cancer, glioblastoma multiforme, cervical cancer, stomach cancer, bladder cancer, hepatoma, breast cancer, colon carcinoma, head and neck cancer, gastric cancer, germ cell tumor, bone cancer, bone tumors, adult malignant fibrous histiocytoma of bone: childhood, malignant fibrous histiocytoma of bone, sarcoma, pediatric sarcoma, sinonasal natural killer, neoplasms, plasma cell neoplasm; myelodysplastic syndromes; neuroblastoma; testicular germ cell tumor, intraocular melanoma, myelodysplastic syndromes; myelodysplastic/myeloproliferative diseases, synovial sarcoma, chronic myeloid leukemia, acute lymphoblastic leukemia, Philadelphia chromosome positive acute lymphoblastic leukemia (Ph+ALL), multiple myeloma, acute myelogenous leukemia, chronic lymphocytic leukemia, mastocytosis and any symptom associated with mastocytosis, and any metastasis thereof. In addition, disorders include urticaria pigmentosa, mastocytosis such as diffuse cutaneous mastocytosis, solitary mastocytoma in human, as well as dog mastocytoma and some rare subtypes like bullous, erythrodermic and telangiectaltic mastocytosis, mastocytosis with an associated hematological disorder, such as a myeloproliferative or myelodysplastic syndrome, or acute leukemia, myeloproliferative disorder associated with mastocytosis, mast cell leukemia, in addition to other cancers. Other cancers are also included within the scope of disorders including, but are not limited to, the following: carcinoma, including that of the bladder, urothelial carcinoma, breast, colon, kidney, liver, lung, ovary, pancreas, stomach, cervix, thyroid, testis, particularly testicular seminomas, and skin; including squamous cell carcinoma; gastrointestinal stromal tumors (“GIST”); hematopoietic tumors of lymphoid lineage, including leukemia, acute lymphocytic leukemia, acute lymphoblastic leukemia. B-cell lymphoma, T-cell lymphoma, Hodgkins lymphoma, non-Hodgkins lymphoma, hairy cell lymphoma and Burketts lymphoma; hematopoietic tumors of myeloid lineage, including acute and chronic myelogenous leukemias and promyelocytic leukemia; tumors of mesenchymal origin, including fibrosarcoma and rhabdomyosarcoma; other tumors, including melanoma, seminoma, teratocarcinoma, neuroblastoma and glioma; tumors of the central and peripheral nervous system, including astrocytoma, neuroblastoma, glioma, and schwannomas; tumors of mesenchymal origin, including fibrosarcoma, rhabdomyosarcoma, and osteosarcoma; and other tumors, including melanoma, xenoderoma pigmentosum, keratoacanthoma, seminoma, thyroid follicular cancer, teratocarcinoma, chemotherapy refractory non-seminomatous germ-cell tumors, and Kaposi's sarcoma, and any metastasis thereof. Other non-limiting examples of types of cancers applicable to the methods encompassed by the present invention include human sarcomas and carcinomas, e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, bone cancer, brain tumor, lung carcinoma (including lung adenocarcinoma), small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma; leukemias. e.g., acute lymphocytic leukemia and acute myelocytic leukemia (myeloblastic, promyelocytic, myelomonocytic, monocytic and erythroleukemia); chronic leukemia (chronic myelocytic (granulocytic) leukemia and chronic lymphocytic leukemia); and polycythemia vera, lymphoma (Hodgkin's disease and non-Hodgkin's disease), multiple myeloma, Waldenstrom's macroglobulinemia, and heavy chain disease. In some embodiments, cancers are epithelial in nature and include but are not limited to, bladder cancer, breast cancer, cervical cancer, colon cancer, gynecologic cancers, renal cancer, laryngeal cancer, lung cancer, oral cancer, head and neck cancer, ovarian cancer, pancreatic cancer, prostate cancer, or skin cancer. In some embodiments, the epithelial cancer is non-small-cell lung cancer, nonpapillary renal cell carcinoma, cervical carcinoma, ovarian carcinoma (e.g., serous ovarian carcinoma), or breast carcinoma. The epithelial cancers may be characterized in various other ways including, but not limited to, serous, endometrioid, mucinous, clear cell. Brenner, or undifferentiated. In some embodiments, the cancer is selected from the group consisting of (advanced) non-small cell lung cancer, melanoma, head and neck squamous cell cancer, (advanced) urothelial bladder cancer, (advanced) kidney cancer (RCC), microsatellite instability-high cancer, classical Hodgkin lymphoma, (advanced) gastric cancer, (advanced) cervical cancer, primary mediastinal B-cell lymphoma, (advanced) hepatocellular carcinoma, colorectal cancer, gastrointestinal cancer, breast invasive carcinoma, bladder urothelial carcinoma, and (advanced) merkel cell carcinoma.

In addition, the compositions described herein may also be administered in combination therapy to further modulate a desired activity. Additional agents include, without limitations, chemotherapeutic agents, hormones, antiangiogenesis, radiolabelled, compounds, or with surgery, cryotherapy, and/or radiotherapy. The preceding treatment methods may be administered in conjunction with other forms of conventional therapy (e.g., standard-of-care treatments for cancer well-known to the skilled artisan), either consecutively with, pre- or post-conventional therapy. For example, these modulatory agents may be administered with a therapeutically effective dose of chemotherapeutic agent. In another embodiment, these modulatory agents are administered in conjunction with chemotherapy to enhance the activity and efficacy of the chemotherapeutic agent. The Physicians' Desk Reference (PDR) discloses dosages of chemotherapeutic agents that have been used in the treatment of various cancers. The dosing regimen and dosages of these aforementioned chemotherapeutic drugs that are therapeutically effective will depend on the particular melanoma, being treated, the extent of the disease and other factors familiar to the physician of skill in the art and may be determined by the physician.

Therapy using one or more compositions described herein, either alone or in combination with other therapies, such as cancer therapies, may be used to contact PRAME-expressing cells and/or administered to a desired subject, such as a subject that is indicated as being a likely responder to therapy. In another embodiment, such therapy may be avoided once a subject is indicated as not being a likely responder to the therapy (e.g., as assessed according to a diagnostic or prognostic method described herein) and an alternative treatment regimen, such as targeted and/or untargeted cancer therapies, may be recommended and/or administered.

The term “targeted therapy” refers to administration of agents that selectively interact with a chosen biomolecule to thereby treat cancer. For example, targeted therapy regarding the inhibition of immune checkpoint inhibitor is useful in combination with the methods encompassed by the present invention.

The term “immunotherapy” or “immunotherapies” generally refers to any strategy for modulating an immune response in a beneficial manner and encompasses the treatment of a subject afflicted with, or at risk of contracting or suffering a recurrence of, a disease by a method comprising inducing, enhancing, suppressing or otherwise modifying an immune response, as well as any treatment that uses certain parts of a subject's immune system to fight diseases, such as cancer. The subject's own immune system is stimulated (or suppressed), with or without administration of one or more agent for that purpose. Immunotherapies that are designed to elicit or amplify an immune response are referred to as “activation immunotherapies.” Immunotherapies that are designed to reduce or suppress an immune response are referred to as “suppression immunotherapies.” In some embodiments, an immunotherapy is specific for cells of interest, such as cancer cells. In some embodiments, immunotherapy may be “untargeted,” which refers to administration of agents that do not selectively interact with immune system cells, yet modulates immune system function. Representative examples of untargeted therapies include, without limitation, chemotherapy, gene therapy, and radiation therapy.

Some forms of immunotherapy are targeted therapies that may comprise, for example, the use of cancer vaccines and/or sensitized antigen presenting cells. For example, an oncolytic virus is a virus that is able to infect and lyse cancer cells, while leaving normal cells unharmed, making them potentially useful in cancer therapy. Replication of oncolytic viruses both facilitates tumor cell destruction and also produces dose amplification at the tumor site. They may also act as vectors for anticancer genes, allowing them to be specifically delivered to the tumor site. The immunotherapy may involve passive immunity for short-term protection of a host, achieved by the administration of pre-formed antibody directed against a cancer antigen or disease antigen (e.g., administration of a monoclonal antibody, optionally linked to a chemotherapeutic agent or toxin, to a tumor antigen). Immunotherapy may also focus on using the cytotoxic lymphocyte-recognized epitopes of cancer cell lines. Alternatively, antisense polynucleotides, ribozymes, RNA interference molecules, triple helix polynucleotides and the like, may be used to selectively modulate biomolecules that are linked to the initiation, progression, and/or pathology of a tumor or cancer. Similarly, immunotherapy may take the form of cell-based therapies. For example, adoptive cellular immunotherapy is a type of immunotherapy using immune cells, such as T cells, that have a natural or genetically engineered reactivity to a patient's cancer are generated and then transferred back into the cancer patient. The injection of a large number of activated tumor-specific T cells may induce complete and durable regression of cancers.

Immunotherapy may involve passive immunity for short-term protection of a host, achieved by the administration of pre-formed antibody directed against a cancer antigen or disease antigen (e.g., administration of a monoclonal antibody, optionally linked to a chemotherapeutic agent or toxin, to a tumor antigen). Immunotherapy may also focus on using the cytotoxic lymphocyte-recognized epitopes of cancer cell lines. Alternatively, antisense polynucleotides, ribozymes, RNA interference molecules, triple helix polynucleotides and the like, may be used to selectively modulate biomolecules that are linked to the initiation, progression, and/or pathology of a tumor or cancer.

In some embodiments, an immunotherapeutic agent is an agonist of an immune-stimulatory molecule; an antagonist of an immune-inhibitory molecule; an antagonist of a chemokine; an agonist of a cytokine that stimulates T cell activation; an agent that antagonizes or inhibits a cytokine that inhibits T cell activation; and/or an agent that binds to a membrane bound protein of the B7 family. In some embodiments, the immunotherapeutic agent is an antagonist of an immune-inhibitory molecule. In some embodiments, the immunotherapeutic agents may be agents for cytokines, chemokines and growth factors, for examples, neutralizing antibodies that neutralize the inhibitory effect of tumor associated cytokines, chemokines, growth factors and other soluble factors, including IL-10, TGF-β and VEGF.

In some embodiments, immunotherapy comprises inhibitors of one or more immune checkpoints. The term “immune checkpoint” refers to a group of molecules on the cell surface of CD4+ and/or CD8+ T cells that fine-tune immune responses by modulating anti-cancer immune responses, such as down-modulating or inhibiting an anti-tumor immune response. Immune checkpoint proteins are well-known in the art and include, without limitation, CTLA-4. PD-1. VISTA, B7-H2, B7-H3, PD-L1. B7-H4, B7-H6, ICOS, HVEM, PD-L2, CD200R, CD160, gp49B, PIR-B, KRLG-1, KIR family receptors, TIM-1, TIM-3, TIM-4, LAG-3 (CD223), IDO, GITR, 4-IBB, OX-40, BTLA, SIRPalpha (CD47), CD48, 2B4 (CD244), B7.1. B7.2, ILT-2, ILT-4, TIGIT, HHLA2, butyrophilins, and A2aR (see, for example, WO 2012/177624). The term further encompasses biologically active protein fragments, as well as nucleic acids encoding full-length immune checkpoint proteins.

Some immune checkpoints are “immune-inhibitory immune checkpoints” encompassing molecules (e.g., proteins) that inhibit, down-regulate, or suppress a function of the immune system (e.g., an immune response). For example, PD-L1 (programmed death-ligand 1), also known as CD274 or B7-H1, is a protein that transmits an inhibitory signal that reduces proliferation of T cells to suppress the immune system. CTLA-4 (cytotoxic T-lymphocyte-associated protein 4), also known as CD152, is a protein receptor on the surface of antigen-presenting cells that serves as an immune checkpoint (“off” switch) to downregulate immune responses. TIM-3 (T-cell immunoglobulin and mucin-domain containing-3), also known as HAVCR2, is a cell surface protein that serves as an immune checkpoint to regulate macrophage activation. VISTA (V-domain Ig suppressor of T cell activation) is a type I transmembrane protein that functions as an immune checkpoint to inhibit T cell effector function and maintain peripheral tolerance. LAG-3 (lymphocyte-activation gene 3) is an immune checkpoint receptor that negatively regulates proliferation, activation, and homeostasis of T cells. BTLA (B- and T-lymphocyte attenuator) is a protein that displays T cell inhibition via interactions with tumor necrosis family receptors (TNF-R). KIR (killer-cell immunoglobulin-like receptor) is a family of proteins expressed on NK cells, and a minority of T cells, that suppress the cytotoxic activity of NK cells. In some embodiments, immunotherapeutic agents may be agents specific to immunosuppressive enzymes such as inhibitors that may block the activities of arginase (ARG) and indoleamine 2,3-dioxygenase (IDO), an immune checkpoint protein that suppresses T cells and NK cells, which change the catabolism of the amino acids arginine and tryptophan in the immunosuppressive tumor microenvironment. The inhibitors may include, but are not limited to, N-hydroxy-L-Arg (NOHA) targeting to ARG-expressing M2 macrophages, nitroaspirin or sildenafil (Viagra®), which blocks ARG and nitric oxide synthase (NOS) simultaneously; and IDO inhibitors, such as 1-methyl-tryptophan. The term further encompasses biologically active protein fragment, as well as nucleic acids encoding full-length immune checkpoint proteins and biologically active protein fragments thereof. In some embodiment, the term further encompasses any fragment according to homology descriptions provided herein.

By contrast, other immune checkpoints are “immune-stimulatory” encompassing molecules (e.g., proteins) that activate, stimulate, or promote a function of the immune system (e.g., an immune response). In some embodiments, the immune-stimulatory molecule is CD28, CD80 (B7.1), CD86 (B7.2), 4-1BB (CD137), 4-1BBL (CD137L). CD27, CD70, CD40, CD40L, CD122, CD226, CD30, CD30L, OX40, OX40L, HVEM, BTLA, GITR and its ligand GITRL, LIGHT, LTβR, LTαβ, ICOS (CD278), ICOSL (B7-H2), and NKG2D. CD40 (cluster of differentiation 40) is a costimulatory protein found on antigen presenting cells that is required for their activation. OX40, also known as tumor necrosis factor receptor superfamily member 4 (TNFRSF4) or CD134, is involved in maintenance of an immune response after activation by preventing T-cell death and subsequently increasing cytokine production. CD137 is a member of the tumor necrosis factor receptor (TNF-R) family that co-stimulates activated T cells to enhance proliferation and T cell survival. CD122 is a subunit of the interleukin-2 receptor (IL-2) protein, which promotes differentiation of immature T cells into regulatory, effector, or memory T cells. CD27 is a member of the tumor necrosis factor receptor superfamily and serves as a co-stimulatory immune checkpoint molecule. CD28 (cluster of differentiation 28) is a protein expressed on T cells that provides co-stimulatory signals required for T cell activation and survival. GITR (glucocorticoid-induced TNFR-related protein), also known as TNFRSF18 and AITR, is a protein that plays a key role in dominant immunological self-tolerance maintained by regulatory T cells. ICOS (inducible T-cell co-stimulator), also known as CD278, is a CD28-superfamily costimulatory molecule that is expressed on activated T cells and play a role in T cell signaling and immune responses.

Immune checkpoints and their sequences are well-known in the art and representative embodiments are described further below. Immune checkpoints generally relate to pairs of inhibitory receptors and the natural binding partners (e.g., ligands). For example, PD-1 polypeptides are inhibitory receptors capable of transmitting an inhibitory signal to an immune cell to thereby inhibit immune cell effector function, or are capable of promoting costimulation (e.g., by competitive inhibition) of immune cells, e.g., when present in soluble, monomeric form. Preferred PD-1 family members share sequence identity with PD-1 and bind to one or more B7 family members, e.g., B7-1, B7-2, PD-1 ligand, and/or other polypeptides on antigen presenting cells. The term “PD-1 activity,” includes the ability of a PD-1 polypeptide to modulate an inhibitory signal in an activated immune cell, e.g., by engaging a natural PD-1 ligand on an antigen presenting cell. Modulation of an inhibitory signal in an immune cell results in modulation of proliferation of, and/or cytokine secretion by, an immune cell. Thus, the term “PD-1 activity” includes the ability of a PD-1 polypeptide to bind its natural ligand(s), the ability to modulate immune cell inhibitory signals, and the ability to modulate the immune response. The term “PD-1 ligand” refers to binding partners of the PD-1 receptor and includes both PD-L1 (Freeman et al. (2000) J. Exp. Med, 192:1027-1034) and PD-L2 (Latchman et al. (2001) Nat. Immunol, 2:261). The term “PD-1 ligand activity” includes the ability of a PD-1 ligand polypeptide to bind its natural receptor(s) (e.g., PD-1 or B7-4), the ability to modulate immune cell inhibitory signals, and the ability to modulate the immune response.

As used herein, the term “immune checkpoint therapy” refers to the use of agents that inhibit immune-inhibitory immune checkpoints, such as inhibiting their nucleic acids and/or proteins. Inhibition of one or more such immune checkpoints may block or otherwise neutralize inhibitory signaling to thereby upregulate an immune response in order to more efficaciously treat cancer. Exemplary agents useful for inhibiting immune checkpoints include antibodies, small molecules, peptides, peptidomimetics, natural ligands, and derivatives of natural ligands, that may either bind and/or inactivate or inhibit immune checkpoint proteins, or fragments thereof; as well as RNA interference, antisense, nucleic acid aptamers, etc. that may downregulate the expression and/or activity of immune checkpoint nucleic acids, or fragments thereof. Exemplary agents for upregulating an immune response include antibodies against one or more immune checkpoint proteins that block the interaction between the proteins and its natural receptor(s); a non-activating form of one or more immune checkpoint proteins (e.g., a dominant negative polypeptide); small molecules or peptides that block the interaction between one or more immune checkpoint proteins and its natural receptor(s); fusion proteins (e.g., the extracellular portion of an immune checkpoint inhibition protein fused to the Fc portion of an antibody or immunoglobulin) that bind to its natural receptor(s); nucleic acid molecules that block immune checkpoint nucleic acid transcription or translation; and the like. Such agents may directly block the interaction between the one or more immune checkpoints and its natural receptor(s) (e.g., antibodies) to prevent inhibitory signaling and upregulate an immune response. Alternatively, agents may indirectly block the interaction between one or more immune checkpoint proteins and its natural receptor(s) to prevent inhibitory signaling and upregulate an immune response. For example, a soluble version of an immune checkpoint protein ligand such as a stabilized extracellular domain may binding to its receptor to indirectly reduce the effective concentration of the receptor to bind to an appropriate ligand. In one embodiment, anti-PD-1 antibodies, anti-PD-L1 antibodies, and/or anti-PD-L2 antibodies, either alone or in combination, are used to inhibit immune checkpoints. Therapeutic agents used for blocking the PD-1 pathway include antagonistic antibodies and soluble PD-L1 ligands. The antagonist agents against PD-1 and PD-L1/2 inhibitory pathway may include, but are not limited to, antagonistic antibodies to PD-1 or PD-L1/2 (e.g., 17D8, 2D3, 4H1, 5C4 (also known as nivolumab or BMS-936558), 4A11, 7D3 and 5F4 disclosed in U.S. Pat. No. 8,008,449; AMP-224, pidilizumab (CT-011), pembrolizumab, and antibodies disclosed in U.S. Pat. Nos. 8,779,105; 8,552,154; 8,217,149; 8,168,757; 8,008,449; 7,488,802; 7,943,743; 7,635,757; and 6,808,710. Similarly, additional representative checkpoint inhibitors may be, but are not limited to, antibodies against inhibitory regulator CTLA-4 (anti-cytotoxic T-lymphocyte antigen 4 anti-cytotoxic T-lymphocyte antigen 4), such as ipilimumab, tremelimumab (fully humanized), anti-CD28 antibodies, anti-CTLA-4 adnectins, anti-CTLA-4 domain antibodies, single chain anti-CTLA-4 antibody fragments, heavy chain anti-CTLA-4 fragments, light chain anti-CTLA-4 fragments, and other antibodies, such as those disclosed in U.S. Pat. Nos. 8,748,815; 8,529,902; 8,318,916; 8,017,114; 7,744,875; 7,605,238; 7,465,446; 7,109,003; 7,132,281; 6,984,720; 6,682,736; 6,207,156; and 5,977,318, as well as EP Pat. No. 1212422. U.S. Pat Publ. Numbers 2002/0039581 and 2002/086,014, and Hurwitz et al. (1998) Proc. Natl. Acad. Sci. U.S.A. 95:10067-10071.

The representative definitions of immune checkpoint activity, ligand, blockade, and the like exemplified for PD-1, PD-L1, PD-L2, and CTLA-4 apply generally to other immune checkpoints.

The term “untargeted therapy” refers to administration of agents that do not selectively interact with a chosen biomolecule yet treat cancer. Representative examples of untargeted therapies include, without limitation, chemotherapy, gene therapy, and radiation therapy.

In one embodiment, chemotherapy is used. Chemotherapy includes the administration of a chemotherapeutic agent. Such a chemotherapeutic agent may be, but is not limited to, those selected from among the following groups of compounds: platinum compounds, cytotoxic antibiotics, antimetabolites, anti-mitotic agents, alkylating agents, arsenic compounds, DNA topoisomerase inhibitors, taxanes, nucleoside analogues, plant alkaloids, and toxins; and synthetic derivatives thereof. Exemplary agents include, but are not limited to, alkylating agents: nitrogen mustards (e.g., cyclophosphamide, ifosfamide, trofosfamide, chlorambucil, estramustine, and melphalan), nitrosoureas (e.g., carmustine (BCNU) and lomustine (CCNU)), alkylsulphonates (e.g., busulfan and treosulfan), triazenes (e.g., dacarbazine, temozolomide), cisplatin, treosulfan, and trofosfamide; plant alkaloids: vinblastine, paclitaxel, docetaxol; DNA topoisomerase inhibitors: teniposide, crisnatol, and mitomycin; anti-folates: methotrexate, mycophenolic acid, and hydroxyurea; pyrimidine analogs: 5-fluorouracil, doxifluridine, and cytosine arabinoside; purine analogs: mercaptopurine and thioguanine; DNA antimetabolites: 2′-deoxy-5-fluorouridine, aphidicolin glycinate, and pyrazoloimidazole; and antimitotic agents: halichondrin, colchicine, and rhizoxin. Similarly, additional exemplary agents including platinum-containing compounds (e.g., cisplatin, carboplatin, oxaliplatin), vinca alkaloids (e.g., vincristine, vinblastine, vindesine, and vinorelbine), taxoids (e.g., paclitaxel or a paclitaxel equivalent such as nanoparticle albumin-bound paclitaxel (ABRAXANE), docosahexaenoic acid bound-paclitaxel (DHA-paclitaxel. Taxoprexin), polyglutamate bound-paclitaxel (PG-paclitaxel, paclitaxel poliglumex, CT-2103, XYOTAX), the tumor-activated prodrug (TAP) ANG1005 (Angiopep-2 bound to three molecules of paclitaxel), paclitaxel-EC-1 (paclitaxel bound to the erhB2-recognizing peptide EC-1), and glucose-conjugated paclitaxel, e.g., 2′-paclitaxel methyl 2-glucopyranosyl succinate; docetaxel, taxol), epipodophyllins (e.g., etoposide, etoposide phosphate, teniposide, topotecan, 9-aminocamptothecin, camptoirinotecan, irinotecan, crisnatol, mytomycin C), anti-metabolites, DHFR inhibitors (e.g., methotrexate, dichloromethotrexate, trimetrexate, edatrexate), IMP dehydrogenase inhibitors (e.g., mycophenolic acid, tiazofurin, ribavirin, and EICAR), ribonucleotide reductase inhibitors (e.g., hydroxyurea and deferoxamine), uracil analogs (e.g., 5-fluorouracil (5-FU), floxuridine, doxifluridine, raltitrexed, tegafur-uracil, capecitabine), cytosine analogs (e.g., cytarabine (ara C), cytosine arabinoside, and fludarabine), purine analogs (e.g., mercaptopurine and Thioguanine), Vitamin D3 analogs (e.g., EB 1089, CB 1093, and KH 1060), isoprenylation inhibitors (e.g., lovastatin), dopaminergic neurotoxins (e.g., 1-methyl-4-phenylpyridinium ion), cell cycle inhibitors (e.g., staurosporine), actinomycin (e.g., actinomycin D, dactinomycin), bleomycin (e.g., bleomycin A2, bleomycin B2, peplomycin), anthracycline (e.g., daunorubicin, doxorubicin, pegylated liposomal doxorubicin, idarubicin, epirubicin, pirarubicin, zorubicin, mitoxantrone), MDR inhibitors (e.g., verapamil), Ca²⁺ ATPase inhibitors (e.g., thapsigargin), imatinib, thalidomide, lenalidomide, tyrosine kinase inhibitors (e.g., axitinib (AG013736), bosutinib (SKI-606), cediranib (RECENTIN™, AZD2171), dasatinib (SPRYCEL®, BMS-354825), erlotinib (TARCEVA®), gefitinib (IRESSA®), imatinib (Gleevec®, CGP57148B, STI-571), lapatinib (TYKERB®, TYVERB®), lestaurtinib (CEP-701), neratinib (HKI-272), nilotinib (TASIGNA®), semaxanib (semaxanib, SU5416), sunitinib (SUTENT®, SU11248), toceranib (PALLADIA®), vandetanib (ZACTIMA®, ZD6474), vatalanib (PTK787, PTK/ZK), trastuzumab (HERCEPTIN®), bevacizumab (AVASTIN®), rituximab (RITUXAN®), cetuximab (ERBITUX®), panitumumab (VECTIBIX®), ranibizumab (Lucentis®), nilotinib (TASIGNA®), sorafenib (NEXAVAR®), everolimus (AFINITOR®), alemtuzumab (CAMPATH®), gemtuzumab ozogamicin (MYLOTARG®), temsirolimus (TORISEL®), ENMD-2076, PCI-32765, AC220, dovitinib lactate (TK1258, CHIR-258), BIBW 2992 (TOVOK™), SGX523, PF-04217903, PF-02341066. PF-299804, BMS-777607, ABT-869, MP470, BIBF 1120 (VARGATEF®). AP24534, JNJ-26483327, MGCD265, DCC-2036, BMS-690154, CEP-11981, tivozanib (AV-951), OSI-930, MM-121, XL-184, XL-647, and/or XL228), proteasome inhibitors (e.g., bortezomib (VELCADE®)), mTOR inhibitors (e.g., rapamycin, temsirolimus (CCI-779), everolimus (RAD-001), ridaforolimus. AP23573 (Ariad), AZD8055 (AstraZeneca), BEZ235 (Novartis), BGT226 (Norvartis), XL765 (Sanofi Aventis), PF-4691502 (Pfizer), GDC0980 (Genentech), SF1126 (Semafoe) and OSI-027 (OSI)), oblimersen, gemcitabine, carminomycin, leucovorin, pemetrexed, cyclophosphamide, dacarbazine, procarbazine, prednisolone, dexamethasone, camptothecin, plicamycin, asparaginase, aminopterin, methopterin, porfiromycin, melphalan, leurosidine, leurosine, chlorambucil, trabectedin, procarbazine, discodermolide, carminomycin, aminopterin, and hexamethyl melamine. Compositions comprising one or more chemotherapeutic agents (e.g., FLAG, CHOP) may also be used. FLAG comprises fludarabine, cytosine arabinoside (Ara-C) and G-CSF. CHOP comprises cyclophosphamide, vincristine, doxorubicin, and prednisone. In another embodiment. PARP (e.g., PARP-1 and/or PARP-2) inhibitors are used and such inhibitors are well-known in the art (e.g., Olaparib, ABT-888, BSI-201, BGP-15 (N-Gene Research Laboratories, Inc.); INO-1001 (Inotek Pharmaceuticals Inc.); PJ34 (Soriano et al., 2001; Pacher et al., 2002b); 3-aminobenzamide (Trevigen); 4-amino-1,8-naphthalimide; (Trevigen); 6(5H)-phenanthridinone (Trevigen); benzamide (U.S. Pat. Re. 36,397); and NU1025 (Bowman et al.). The mechanism of action is generally related to the ability of PARP inhibitors to bind PARP and decrease its activity. PARP catalyzes the conversion of beta-nicotinamide adenine dinucleotide (NAD+) into nicotinamide and poly-ADP-ribose (PAR). Both poly (ADP-ribose) and PARP have been linked to regulation of transcription, cell proliferation, genomic stability, and carcinogenesis (Bouchard et. al. (2003) Exp. Hematol, 31:446-454); Herceg (2001) Mut. Res, 477:97-110). Poly(ADP-ribose) polymerase 1 (PARP1) is a key molecule in the repair of DNA single-strand breaks (SSBs) (de Murcia J. et al. (1997) Proc. Natl. Acad. Sci. U.S.A, 94:7303-7307; Schreiber et al. (2006) Nat. Rev. Mol. Cell Biol, 7:517-528; Wang et al. (1997) Genes Dev, 11:2347-2358). Knockout of SSB repair by inhibition of PARP1 function induces DNA double-strand breaks (DSBs) that may trigger synthetic lethality in cancer cells with defective homology-directed DSB repair (Bryant et al. (2005) Nature 434:913-917; Farmer et al. (2005) Nature 434:917-921). The foregoing examples of chemotherapeutic agents are illustrative and are not intended to be limiting.

In another embodiment, radiation therapy is used. The radiation used in radiation therapy may be ionizing radiation. Radiation therapy may also be gamma rays, X-rays, or proton beams. Examples of radiation therapy include, but are not limited to, external-beam radiation therapy, interstitial implantation of radioisotopes (1-125, palladium, iridium), radioisotopes such as strontium-89, thoracic radiation therapy, intraperitoneal P-32 radiation therapy, and/or total abdominal and pelvic radiation therapy. For a general overview of radiation therapy, see Hellman. Chapter 16: Principles of Cancer Management: Radiation Therapy, 6th edition, 2001, DeVita et al., eds., J. B. Lippencott Company, Philadelphia. The radiation therapy may be administered as external beam radiation or teletherapy wherein the radiation is directed from a remote source. The radiation treatment may also be administered as internal therapy or brachytherapy wherein a radioactive source is placed inside the body close to cancer cells or a tumor mass. Also encompassed is the use of photodynamic therapy comprising the administration of photosensitizers, such as hematoporphyrin and its derivatives, Verteporfin (BPD-MA), phthalocyanine, photosensitizer Pc4, demethoxy-hypocrellin A; and 2BA-2-DMHA.

In another embodiment, hormone therapy is used. Hormonal therapeutic treatments may comprise, for example, hormonal agonists, hormonal antagonists (e.g., flutamide, bicalutamide, tamoxifen, raloxifene, leuprolide acetate (LUPRON), LH-RH antagonists), inhibitors of hormone biosynthesis and processing, and steroids (e.g., dexamethasone, retinoids, deltoids, betamethasone, cortisol, cortisone, prednisone, dehydrotestosterone, glucocorticoids, mineralocorticoids, estrogen, testosterone, progestins), vitamin A derivatives (e.g., all-trans retinoic acid (ATRA)); vitamin D3 analogs; antigestagens (e.g., mifepristone, onapristone), or antiandrogens (e.g., cyproterone acetate).

In another embodiment, hyperthermia, a procedure in which body tissue is exposed to high temperatures (up to 106° F.) is used. Heat may help shrink tumors by damaging cells or depriving them of substances they need to live. Hyperthermia therapy may be local, regional, and whole-body hyperthermia, using external and internal heating devices. Hyperthermia is almost always used with other forms of therapy (e.g., radiation therapy, chemotherapy, and biological therapy) to try to increase their effectiveness. Local hyperthermia refers to heat that is applied to a very small area, such as a tumor. The area may be heated externally with high-frequency waves aimed at a tumor from a device outside the body. To achieve internal heating, one of several types of sterile probes may be used, including thin, heated wires or hollow tubes filled with warm water: implanted microwave antennae; and radiofrequency electrodes. In regional hyperthermia, an organ or a limb is heated. Magnets and devices that produce high energy are placed over the region to be heated. In another approach, called perfusion, some of the patient's blood is removed, heated, and then pumped (perfused) into the region that is to be heated internally. Whole-body heating is used to treat metastatic cancer that has spread throughout the body. It may be accomplished using warm-water blankets, hot wax, inductive coils (like those in electric blankets), or thermal chambers (similar to large incubators). Hyperthermia does not cause any marked increase in radiation side effects or complications. Heat applied directly to the skin, however, may cause discomfort or even significant local pain in about half the patients treated. It may also cause blisters, which generally heal rapidly.

In still another embodiment, photodynamic therapy (also called PDT, photoradiation therapy, photo therapy, or photochemotherapy) is used for the treatment of some types of cancer. It is based on the discovery that certain chemicals known as photosensitizing agents may kill one-celled organisms when the organisms are exposed to a particular type of light. PDT destroys cancer cells through the use of a fixed-frequency laser light in combination with a photosensitizing agent. In PDT, the photosensitizing agent is injected into the bloodstream and absorbed by cells all over the body. The agent remains in cancer cells for a longer time than it does in normal cells. When the treated cancer cells are exposed to laser light, the photosensitizing agent absorbs the light and produces an active form of oxygen that destroys the treated cancer cells. Light exposure must be timed carefully so that it occurs when most of the photosensitizing agent has left healthy cells but is still present in the cancer cells. The laser light used in PDT may be directed through a fiber-optic (a very thin glass strand). The fiber-optic is placed close to the cancer to deliver the proper amount of light. The fiber-optic may be directed through a bronchoscope into the lungs for the treatment of lung cancer or through an endoscope into the esophagus for the treatment of esophageal cancer. An advantage of PDT is that it causes minimal damage to healthy tissue. However, because the laser light currently in use cannot pass through more than about 3 centimeters of tissue (a little more than one and an eighth inch). PDT is mainly used to treat tumors on or just under the skin or on the lining of internal organs. Photodynamic therapy makes the skin and eyes sensitive to light for 6 weeks or more after treatment. Patients are advised to avoid direct sunlight and bright indoor light for at least 6 weeks. If patients must go outdoors, they need to wear protective clothing, including sunglasses. Other temporary side effects of PDT are related to the treatment of specific areas and may include coughing, trouble swallowing, abdominal pain, and painful breathing or shortness of breath. In December 1995, the U.S. Food and Drug Administration (FDA) approved a photosensitizing agent called porfimer sodium, or Photofrin®, to relieve symptoms of esophageal cancer that is causing an obstruction and for esophageal cancer that cannot be satisfactorily treated with lasers alone. In January 1998, the FDA approved porfimer sodium for the treatment of early nonsmall cell lung cancer in patients for whom the usual treatments for lung cancer are not appropriate. The National Cancer Institute and other institutions are supporting clinical trials (research studies) to evaluate the use of photodynamic therapy for several types of cancer, including cancers of the bladder, brain, larynx, and oral cavity.

In yet another embodiment, laser therapy is used to harness high-intensity light to destroy cancer cells. This technique is often used to relieve symptoms of cancer such as bleeding or obstruction, especially when the cancer cannot be cured by other treatments. It may also be used to treat cancer by shrinking or destroying tumors. The term “laser” stands for light amplification by stimulated emission of radiation. Ordinary light, such as that from a light bulb, has many wavelengths and spreads in all directions. Laser light, on the other hand, has a specific wavelength and is focused in a narrow beam. This type of high-intensity light contains a lot of energy. Lasers are very powerful and may be used to cut through steel or to shape diamonds. Lasers also may be used for very precise surgical work, such as repairing a damaged retina in the eye or cutting through tissue (in place of a scalpel). Although there are several different kinds of lasers, only three kinds have gained wide use in medicine: Carbon dioxide (CO₂) laser—This type of laser may remove thin layers from the skin's surface without penetrating the deeper layers. This technique is particularly useful in treating tumors that have not spread deep into the skin and certain precancerous conditions. As an alternative to traditional scalpel surgery, the CO₂ laser is also able to cut the skin. The laser is used in this way to remove skin cancers. Neodymium:yttrium-aluminum-garnet (Nd:YAG) laser—Light from this laser may penetrate deeper into tissue than light from the other types of lasers, and it may cause blood to clot quickly. It may be carried through optical fibers to less accessible parts of the body. This type of laser is sometimes used to treat throat cancers. Argon laser—This laser may pass through only superficial layers of tissue and is therefore useful in dermatology and in eye surgery. It also is used with light-sensitive dyes to treat tumors in a procedure known as photodynamic therapy (PDT). Lasers have several advantages over standard surgical tools, including: Lasers are more precise than scalpels. Tissue near an incision is protected, since there is little contact with surrounding skin or other tissue. The heat produced by lasers sterilizes the surgery site, thus reducing the risk of infection. Less operating time may be needed because the precision of the laser allows for a smaller incision. Healing time is often shortened; since laser heat seals blood vessels, there is less bleeding, swelling, or scarring. Laser surgery may be less complicated. For example, with fiber optics, laser light may be directed to parts of the body without making a large incision. More procedures may be done on an outpatient basis. Lasers may be used in two ways to treat cancer: by shrinking or destroying a tumor with heat, or by activating a chemical—known as a photosensitizing agent—that destroys cancer cells. In PDT, a photosensitizing agent is retained in cancer cells and may be stimulated by light to cause a reaction that kills cancer cells. CO₂ and Nd:YAG lasers are used to shrink or destroy tumors. They may be used with endoscopes, tubes that allow physicians to see into certain areas of the body, such as the bladder. The light from some lasers may be transmitted through a flexible endoscope fitted with fiber optics. This allows physicians to see and work in parts of the body that could not otherwise be reached except by surgery and therefore allows very precise aiming of the laser beam. Lasers also may be used with low-power microscopes, giving the doctor a clear view of the site being treated. Used with other instruments, laser systems may produce a cutting area as small as 200 microns in diameter—less than the width of a very fine thread. Lasers are used to treat many types of cancer. Laser surgery is a standard treatment for certain stages of glottis (vocal cord), cervical, skin, lung, vaginal, vulvar, and penile cancers. In addition to its use to destroy the cancer, laser surgery is also used to help relieve symptoms caused by cancer (palliative care). For example, lasers may be used to shrink or destroy a tumor that is blocking a patient's trachea (windpipe), making it easier to breathe. It is also sometimes used for palliation in colorectal and anal cancer. Laser-induced interstitial thermotherapy (LITT) is one of the most recent developments in laser therapy. LITT uses the same idea as a cancer treatment called hyperthermia; that heat may help shrink tumors by damaging cells or depriving them of substances they need to live. In this treatment, lasers are directed to interstitial areas (areas between organs) in the body. The laser light then raises the temperature of the tumor, which damages or destroys cancer cells.

In one aspect, provided herein is a method of eliciting in a subject an immune response to a cell that expresses a PRAME antigen. In some embodiments, the method comprises administering to the subject a pharmaceutical composition described herein, wherein the pharmaceutical composition, when administered to the subject, elicits an immune response to the cell that expresses a PRAME antigen.

In some embodiments, the immune response can include a cell-mediated immune response. A cellular immune response is a response that involves T cells and may be determined in vitro, ex vivo, or in vivo. For example, a general cellular immune response may be determined as the T cell proliferative activity in cells (e.g., peripheral blood leukocytes (PBLs)) sampled from the subject at a suitable time following the administering of a pharmaceutical composition. Following incubation of e.g., PBMCs with a stimulator for an appropriate period, [³H]thymidine incorporation may be determined. The subset of T cells that is proliferating may be determined using flow cytometry.

In another aspect encompassed by the present invention, the methods provided herein include administering to both human and non-human mammals as described above. Veterinary applications also are contemplated. In some embodiments, the subject may be any living organism in which an immune response may be elicited.

In some embodiments, the pharmaceutical composition may be administered at any time that is appropriate. For example, the administering may be conducted before or during treatment of a subject having a disorder characterized by PRAME expression (e.g., a non-malignant disorder, a hyperproliferative disorder, or a relapse of a hyperproliferative disorder characterized by PRAME expression), and continued after the disorder characterized by expression of a PRAME antigen becomes clinically undetectable. The administering also may be continued in a subject showing signs of recurrence.

In some embodiments, the pharmaceutical composition may be administered in a therapeutically or a prophylactically effective amount. Administering the pharmaceutical composition to the subject may be carried out using known procedures, and at dosages and for periods of time sufficient to achieve a desired effect.

In some embodiments, the pharmaceutical composition may be administered to the subject at any suitable site. Administration may be accomplished using methods generally known in the art. Agents, including cells, may be introduced to the desired site by direct injection, or by any other means used in the art including, but are not limited to, intravascular, intracerebral, parenteral, intraperitoneal, intravenous, epidural, intraspinal, intrasternal, intraarticular, intra-synovial, intrathecal, intra-arterial, intracardiac, or intramuscular administration. For example, subjects of interest may be engrafted with the transplanted cells by various routes. Such routes include, but are not limited to, intravenous administration, subcutaneous administration, administration to a specific tissue (e.g., focal transplantation), injection into the femur bone marrow cavity, injection into the spleen, administration under the renal capsule of fetal liver, and the like. In certain embodiment, the cancer vaccine encompassed by the present invention is injected to the subject intratumorally or subcutaneously. Cells may be administered in one infusion, or through successive infusions over a defined time period sufficient to generate a desired effect. Exemplary methods for transplantation, engraftment assessment, and marker phenotyping analysis of transplanted cells are well-known in the art (see, for example, Pearson et al. (2008) Curr. Protoc. Immunol, 81:15.21.1-15.21.21; Ito et al. (2002) Blood 100:3175-3182; Traggiai et al. (2004) Science 304:104-107; Ishikawa et al. Blood (2005) 106:1565-1573; Shultz et al. (2005) J. Immunol, 174:6477-6489; and Holyoake et al. (1999) Exp. Hematol, 27:1418-1427). In some embodiments, the dose may be administered in an amount and for a period of time effective in bringing about a desired response, be it eliciting the immune response or the prophylactic or therapeutic treatment of a disorder characterized by PRAME expression (e.g., a non-malignant disorder, a hyperproliferative disorder, or a relapse of a hyperproliferative disorder characterized by PRAME expression) and/or symptoms associated therewith.

The pharmaceutical composition may be given subsequent to, preceding, or contemporaneously with other therapies including therapies that also elicit an immune response in the subject. For example, the subject may previously or concurrently be treated by other forms of immunomodulatory agents, such other therapies may be provided in such a way so as not to interfere with the immunogenicity of the compositions described herein.

Administering may be properly timed by the care giver (e.g., physician, veterinarian), and may depend on the clinical condition of the subject, the objectives of administering, and/or other therapies also being contemplated or administered. In some embodiments, an initial dose may be administered, and the subject monitored for an immunological and/or clinical response. Suitable means of immunological monitoring include using patient's peripheral blood lymphocyte (PBL) as responders and immunogenic peptides or peptide-MHC complexes described herein as stimulators. An immunological reaction also may be determined by a delayed inflammatory response at the site of administering. One or more doses subsequent to the initial dose may be given as appropriate, typically on a monthly, semimonthly, or a weekly basis, until the desired effect is achieved. Thereafter, additional booster or maintenance doses may be given as required, particularly when the immunological or clinical benefit appears to subside.

In general, an appropriate dosage and treatment regimen provides the active molecules or cells in an amount sufficient to provide a benefit. Such a response may be monitored by establishing an improved clinical outcome (e.g., more frequent remissions, complete or partial, or longer disease-free survival) in treated subjects as compared to non-treated subjects. Increases in preexisting immune responses to a viral protein generally correlate with an improved clinical outcome. Such immune responses may generally be evaluated using standard proliferation, cytotoxicity or cytokine assays, which are routine.

For prophylactic use, a dose should be sufficient to prevent, delay the onset of, or diminish the severity of a disease associated with disease or disorder. Prophylactic benefit of the immunogenic compositions administered according to the methods described herein can be determined by performing pre-clinical (including in vitro, ex vivo, and in vivo animal studies) and clinical studies and analyzing data obtained therefrom by appropriate statistical, biological, and clinical methods and techniques, all of which can readily be practiced by an ordinarily skilled artisan.

As used herein, administration of a composition refers to delivering the same to a subject, regardless of the route or mode of delivery. Administration may be effected continuously or intermittently, and parenterally. Administration may be for treating a subject already confirmed as having a recognized condition, disease or disease state, or for treating a subject susceptible to or at risk of developing such a condition, disease or disease state. Co-administration with an adjunctive therapy may include simultaneous and/or sequential delivery of multiple agents in any order and on any dosing schedule (e.g., engineered immune cells with one or more cytokines; immunosuppressive therapy such as calcineurin inhibitors, corticosteroids, microtubule inhibitors, low dose of a mycophenolic acid prodrug, or any combination thereof).

In some embodiments, a plurality of doses of a host cell (e.g., an engineered immune cell) described herein is administered to the subject, which may be administered at intervals between administrations of about two to about four weeks.

Treatment or prevention methods encompassed by the present invention may be administered to a subject as part of a treatment course or regimen, which may comprise additional treatments prior to, or after, administration of the instantly disclosed unit doses, cells, or compositions. For example, in some embodiments, a subject receiving a unit dose of the host cell (e.g., an engineered immune cell) is receiving or had previously received a hematopoietic cell transplant (HCT; including myeloablative and non-myeloablative HCT). In any of the foregoing embodiments, a hematopoietic cell used in an HCT may be a “universal donor” cell that is modified to reduce or eliminate expression of one or more endogenous genes that encode a polypeptide product selected from an MHC, antigen, and a binding protein (e.g., by a chromosomal gene knockout according to the methods described herein).

Techniques and regimens for performing cell transplantation are known in the art and may comprise transplantation of any suitable donor cell, such as a cell derived from umbilical cord blood, bone marrow, or peripheral blood, a hematopoietic stem cell, a mobilized stem cell, or a cell from amniotic fluid. Accordingly, in some embodiments, a host cell (e.g., an engineered immune cell) encompassed by the present invention may be administered with or shortly after stem cell therapy.

Methods encompassed by the present invention may, in some embodiments, further include administering one or more additional agents to treat the disease or disorder (e.g., a disorder characterized by PRAME expression such as a non-malignant disorder, a hyperproliferative disorder, or a relapse of a hyperproliferative disorder characterized by PRAME expression) in a combination therapy. For example, in some embodiments, a combination therapy comprises administering host cell or binding protein encompassed by the present invention with (concurrently, simultaneously, or sequentially) an antiviral agent. In some embodiments, a combination therapy comprises administering a host cell or binding protein encompassed by the present invention with lopinavir/ritonavir, chloroquine, ribavirin, steroid drugs, hydroxychloroquine, and/or interferon α. In some embodiments, a combination therapy comprises administering a host cell, composition, or unit dose of the host cells encompassed by the present invention with a secondary therapy, such as a surgery, an antibody, a vaccine, or any combination thereof.

In some embodiments, the subject is a human, such as a human with a disorder characterized by PRAME expression (e.g., a non-malignant disorder, a hyperproliferative disorder, or a relapse of a hyperproliferative disorder characterized by PRAME expression). In some embodiments, the subject is a rodent, such as a mouse. In some such embodiments, the mouse is a transgenic mouse, such as a mouse expressing human MHC (i.e., HLA) molecules, such as HLA-A2 (e.g., Nicholson et al. (2012) Adv. Hematol, 2012:404081).

In some embodiments, the subject is a transgenic mouse expressing human TCRs or is an antigen-negative mouse (e.g., Li et al (2010) Nat. Med, 16:1029-1034 and Obenaus et al. (2015) Nat. Biotechnol, 33:402-407). In some embodiments, the subject is a transgenic mouse expressing human HLA molecules and human TCRs.

In some embodiments, such as where the subject is a transgenic HLA mouse, the identified TCRs are modified. e.g., to be chimeric or humanized. In some embodiments, the TCR scaffold is modified, such as analogous to known binding protein humanizing methods.

c. Screening Methods

Another aspect encompassed by the present invention encompasses screening assays.

In some embodiments, methods are provided for selecting agents that bind to a PRAME immunogenic peptide or pMHC described herein. For example, a method of identifying a peptide-binding molecule, or antigen-binding fragment thereof, that binds to a peptide epitope selected from the peptide sequences listed in Table 1 comprising a) providing a cell presenting a peptide epitope selected from the peptide sequences listed in Table 1 in the context of an MHC molecule on the surface of the cell; b) determining binding of a plurality of candidate peptide-binding molecules or antigen-binding fragments thereof to the peptide epitope in the context of the MHC molecule on the cell; and c) identifying one or more peptide-binding molecules or antigen-binding fragments thereof that bind to the peptide epitope in the context of the MHC molecule, is provided.

In some embodiments, a method of identifying a peptide-binding molecule or antigen-binding fragment thereof that binds to a peptide epitope selected from the peptide sequences listed in Table 1 comprising: a) providing a peptide epitope either alone or in a stable MHC-peptide complex, comprising a peptide epitope selected from the peptide sequences listed in Table 1, either alone or in the context of an MHC molecule; b) determining binding of a plurality of candidate peptide-binding molecules or antigen-binding fragments thereof to the peptide or stable MHC-peptide complex; and c) identifying one or more peptide-binding molecules or antigen-binding fragments thereof that bind to the peptide epitope or the stable MHC-peptide complex, optionally wherein the MHC or MHC-peptide complex is as described herein, is provided.

In some embodiments, provide herein are methods of identifying a peptide-binding molecule or antigen-binding fragment thereof that binds to a peptide epitope selected from Table 1.

In some embodiments, the peptide binding molecule (e.g., MHC-peptide binding molecule) is a molecule or portion thereof that possesses the ability to bind (e.g., specifically and/or selectively) to a peptide epitope that is presented or displayed in the context of an MHC molecule (MHC-peptide complex), such as on the surface of a cell. Exemplary peptide binding molecules include T cell receptors or antibodies, or antigen-binding portions thereof, including single chain immunoglobulin variable regions (e.g., scTCR, scFv) thereof, that exhibit specific ability to bind to an MHC-peptide complex. In some embodiments, the peptide binding molecule is a TCR or antigen-binding fragment thereof. In some embodiments, the peptide binding molecule is an antibody, such as a TCR-like antibody or antigen-binding fragment thereof. In some embodiments, the peptide binding molecule is a TCR-like CAR that contains an antibody or antigen binding fragment thereof, such as a TCR-like antibody, such as one that has been engineered to bind to MHC-peptide complexes. In some embodiments, the peptide binding molecule may be derived from natural sources, or it may be partly or wholly synthetically or recombinantly produced.

In some embodiments, a binding molecule that binds to a peptide epitope may be identified by contacting one or more candidate peptide binding molecules, such as one or more candidate TCR molecules, antibodies, or antigen-binding fragments thereof, with an MHC-peptide complex, and assessing whether each of the one or more candidate binding molecules binds (e.g., specifically and/or selectively) to the MHC-peptide complex. The methods may be performed in vitro, ex vivo, or in vivo. Methods are well-known in the art for screening, such as described in U.S. Pat. Publ, 2020/0102553.

In some embodiments, the methods include contacting a plurality or library of binding molecules, such as a plurality or library of TCRs or antibodies, with an MHC-restricted epitope and identifying or selecting molecules that specifically and/or selectively bind such an epitope. In some embodiments, a library or collection containing a plurality of different binding molecules, such as a plurality of different TCRs or a plurality of different antibodies, may be screened or assessed for binding to an MHC-restricted epitope. In some embodiments, such as for selecting a binding protein that specifically and/or selectively binds an MHC-restricted peptide, hybridoma methods may be employed.

In some embodiments, screening methods may be employed in which a plurality of candidate binding molecules, such as a library or collection of candidate binding molecules, are individually contacted with an peptide binding molecule, either simultaneously or sequentially. Library members that specifically and/or selectively bind to a particular MHC-peptide complex may be identified or selected. In some embodiments, the library or collection of candidate binding molecules may contain at least 2, 5, 10, 100, 10³, 10⁴, 10⁵, 10⁶, 10⁷, 10⁸, 10⁹, or more different peptide binding molecules.

In some embodiments, the methods may be employed to identify a peptide binding molecule, such as a TCR or an antibody, that exhibits binding for more than one MHC haplotype or more than one MHC allele. In some embodiments, the peptide binding molecule, such as a TCR or antibody, specifically and/or selectively binds or recognizes a peptide epitope presented in the context of a plurality of MHC class I haplotypes or alleles. In some embodiments, the peptide binding molecule, such as a TCR or antibody, specifically and/or selectively binds or recognizes a peptide epitope presented in the context of a plurality of MHC class II haplotypes or alleles.

A variety of assays are known for assessing binding affinity and/or determining whether a binding molecule specifically and/or selectively binds to a particular ligand (e.g., MHC-peptide complex). It is within the level of a skilled artisan to determine the binding affinity of a TCR for a T cell epitope of a target polypeptide, such as by using any of a number of binding assays that are well-known in the art. For example, in some embodiments, a Biacore® machine may be used to determine the binding constant of a complex between two proteins. The dissociation constant (K_{D}) for the complex may be determined by monitoring changes in the refractive index with respect to time as buffer is passed over the chip. Other suitable assays for measuring the binding of one protein to another include, for example, immunoassays such as enzyme linked immunosorbent assays (ELISA) and radioimmunoassays says (RIA), or determination of binding by monitoring the change in the spectroscopic or optical properties of the proteins through fluorescence, UV absorption, circular dichroism, or nuclear magnetic resonance (NMR). Other exemplary assays include, but are not limited to, Western blot, ELISA, analytical ultracentrifugation, spectroscopy and surface plasmon resonance (Biacore®) analysis (see. e.g., Scatchard et al. (1949) Ann. N. Y. Acad. Sci, 51:660; Wilson (2002) Science 295:2103; Wolff et al. (1993) Cancer Res. 53:2560; and U.S. Pat. Nos. 5,283,173, 5,468,614, or the equivalent), flow cytometry, sequencing and other methods for detection of expressed nucleic acids. In one example, apparent affinity for a TCR is measured by assessing binding to various concentrations of tetramers, for example, by flow cytometry using labeled tetramers. In one example, apparent K_{D} of a TCR is measured using 2-fold dilutions of labeled tetramers at a range of concentrations, followed by determination of binding curves by non-linear regression, apparent K_{D} being determined as the concentration of ligand that yielded half-maximal binding.

In some embodiments, the methods may be used to identify binding molecules that bind only if the particular peptide is present in the complex, and not if the particular peptide is absent or if another, non-overlapping or unrelated peptide is present. In some embodiments, the binding molecule does not substantially bind the MHC in the absence of the bound peptide, and/or does not substantially bind the peptide in the absence of the MHC. In some embodiments, the binding molecules are at least partially specific. In some embodiments, an exemplary identified binding molecule may bind to an MHC-peptide complex if the particular peptide is present, and also bind if a related peptide that has one or two substitutions relative to the particular peptide is present.

In some embodiments, an identified antibody, such as a TCR-like antibody, may be used to produce or generate a chimeric antigen receptors (CARs) containing a non-TCR antibody that specifically and/or selectively binds to a MHC-peptide complex.

In some embodiments, the methods of identifying a peptide binding molecule, such as a TCR or TCR-like antibody or TCR-like CAR, may be used to engineer cells expressing or containing a peptide binding molecule. In some embodiments, a cell or engineered cell is a T cell. In some embodiments, the T cell is a CD4+ or CD8+ T cell. In some embodiments, the peptide binding molecule recognizes a MHC class I peptide complex, an MHC class II peptide complex and/or an MHC-E peptide complex. In some embodiments, a peptide binding molecule, such as a TCR or antibody or CAR, that specifically and/or selectively recognizes a peptide in the context of an MHC class I may be used to engineer CD8+ T cells. In some embodiments, also provided is a composition of engineered CD8+ T cells expressing or containing the TCR, antibody or CAR, for recognition of a peptide presented in the context of MHC class I. In any of such embodiments, the cells may be used in methods of adoptive cell therapy.

In some embodiments. TCR libraries may be generated by amplification of the repertoire of Vα and Vβ from T cells isolated from a subject, including cells present in PBMCs, spleen or other lymphoid organ. In some cases, T cells may be amplified from tumor-infiltrating lymphocytes (TILs). In some embodiments. TCR libraries may be generated from CD4+ or CD8+ cells. In some embodiments, the TCRs may be amplified from a T cell source of a normal of healthy subject, i.e., normal TCR libraries. In some embodiments, the TCRs may be amplified from a T cell source of a diseased subject, i.e., diseased TCR libraries. In some embodiments, degenerate primers are used to amplify the gene repertoire of Vα and VP, such as by RT-PCR in samples, such as T cells, obtained from humans. In some embodiments, scTv libraries may be assembled from naive Vα and Vβ libraries in which the amplified products are cloned or assembled to be separated by a linker. Depending on the source of the subject and cells, the libraries may be HLA allele-specific.

Alternatively, in some embodiments, TCR libraries may be generated by mutagenesis or diversification of a parent or scaffold TCR molecule. For example, in some aspects, a subject, e.g., human or other mammal such as a rodent, may be vaccinated with a peptide, such as a peptide identified by the present methods. In some embodiments, a sample may be obtained from the subject, such as a sample containing blood lymphocytes. In some instances, binding molecules, e.g., TCRs, may be amplified out of the sample, e.g., T cells contained in the sample. In some embodiments, antigen-specific T cells may be selected, such as by screening to assess CTL activity against the peptide. In some aspects, TCRs, e.g., present on the antigen-specific T cells, may be selected, such as by binding activity, e.g., particular affinity or avidity for the antigen. In some aspects, the TCRs are subjected to directed evolution, such as by mutagenesis, e.g., of the α or β chain. In some aspects, particular residues within CDRs of the TCR are altered. In some embodiments, selected TCRs may be modified by affinity maturation. In some aspects, a selected TCR may be used as a parent scaffold TCR against the antigen.

In some embodiments, the subject is a human, such as a human with a disorder characterized by PRAME expression. In some embodiments, the subject is a rodent, such as a mouse. In some such embodiments, the mouse is a transgenic mouse, such as a mouse expressing human MHC (i.e., HLA) molecules, such as HLA-A2 (e.g., Nicholson et al. (2012) Adv. Hematol, 2012:404081).

In some embodiments, the subject is a transgenic mouse expressing human TCRs or is an antigen-negative mouse (e.g., Li et al. (2010) Nat Med, 161029-1034; Obenaus et al. (2015) Nat. Biotechnol, 33:402-407). In some embodiments, the subject is a transgenic mouse expressing human HLA molecules and human TCRs.

In some embodiments, such as where the subject is a transgenic HLA mouse, the identified TCRs are modified, e.g., to be chimeric or humanized. In some aspects, the TCR scaffold is modified, such as analogous to known antibody humanizing methods.

In some embodiments, such a scaffold molecule is used to generate a library of TCRs.

For example, in some embodiments, the library includes TCRs or antigen-binding portions thereof that have been modified or engineered compared to the parent or scaffold TCR molecule. In some embodiments, directed evolution methods may be used to generate TCRs with altered properties, such as with higher affinity for a specific MHC-peptide complex. In some embodiments, display approaches involve engineering, or modifying, a known, parent or reference TCR. For example, in some cases, a wild-type TCR may be used as a template for producing mutagenized TCRs in which in one or more residues of the CDRs are mutated, and mutants with an desired altered property, such as higher affinity for a desired target antigen, are selected. In some embodiments, directed evolution is achieved by display methods including, but not limited to, yeast display (Holler et al. (2003) Nat. Immunol, 4:55-62; Holler et al. (2000) Proc. Natl. Acad Sci. U.S.A, 97:5387-5392), phage display (Li et al. (2005) Nat. Biotechnol, 23:349-354), or T cell display (Chervin et al. (2008) J. Immunol. Methods 339:175-184).

In some embodiments, the libraries may be soluble. In some embodiments, the libraries are display libraries in which the TCR is displayed on the surface of a phage or cell, or attached to a particle or molecule, such as a cell, ribosome or nucleic acid, e.g., RNA or DNA. Typically, the TCR libraries, including normal and disease TCR libraries or diversified libraries, may be generated in any form, including as a heterodimer or as a single chain form. In some embodiments, one or more members of the TCR may be a two-chain heterodimer. In some embodiments, pairing of the Vα and Vβ chains may be promoted by introduction of a disulfide bond. In some embodiments, members of the TCR library may be a TCR single chain (scTv or ScTCR), which, in some cases, may include a Vα and Vβ chain separated by a linker. Further, in some cases, upon screening and selection of a TCR from the library, the selected member may be generated in any form, such as a full-length TCR heterodimer or single-chain form or as antigen-binding fragments thereof.

Other methods of identifying molecules that bind to a peptide in the context of an MHC molecule are also described in U.S. Pat. Appl. No. 2020/0182884.

More generally, the present invention encompasses assays for screening agents, such as test proteins, that bind to, or modulate the activity of, PRAME or an antigen thereof. Such agents include, without limitation, antibodies, proteins, fusion proteins, small molecules, and nucleic acids. In some embodiments, a method for identifying an agent which modulates an immune response entails determining the ability of the candidate agent to modulate PRAME activity and further modulate an immune response of interest, such as modulated cytotoxic T cell activation and/or activity, sensitivity of cancer cells to immune checkpoint therapy, and the like.

In some embodiments, an assay is a cell-free or cell-based assay, comprising contacting a target, with a test agent, and determining the ability of the test agent to modulate (e.g., upregulate or downregulate) the amount and/or activity of the target, such as by measuring direct or indirect parameters as described below.

In some embodiments, an assay is a cell-based assay, such as one comprising contacting (a) a cell of interest with a test agent and determining the ability of the test agent to modulate the amount and/or activity of the target, such as binding characteristics. Determining the ability of the polypeptides to bind to, or interact with, each other may be accomplished, e.g., by measuring direct binding or by measuring a parameter of immune cell activation or function.

In another embodiment, an assay is a cell-based assay, comprising contacting a cell such as a cancer cell with immune cells (e.g., cytotoxic T cells) and a test agent, and determining the ability of the test agent to modulate the amount and/or activity of the target, and/or modulated immune responses, such as by measuring direct or indirect parameters as described below.

The methods described above and herein may also be adapted to test one or more agents that are already known to modulate the amount and/or activity of one or more biomarkers described herein to confirm modulation of the one or more biomarkers and/or to confirm the effects of the agents on readouts of a desired phenotype, such as modulated immune responses, sensitivity to immune checkpoint blockade, and the like.

In a direct binding assay, biomarker protein (or their respective target polypeptides or molecules) may be coupled with a radioisotope or enzymatic label such that binding may be determined by detecting the labeled protein or molecule in a complex. For example, the targets may be labeled with ¹²⁵I, ³⁵S, ¹⁴C, or ³H, either directly or indirectly, and the radioisotope detected by direct counting of radio emmission or by scintillation counting. Alternatively, the targets may be enzymatically labeled with, for example, horseradish peroxidase, alkaline phosphatase, or luciferase, and the enzymatic label detected by determination of conversion of an appropriate substrate to product. Determining the interaction between target and substrate may also be accomplished using standard binding or enzymatic analysis assays. In one or more embodiments of the above described assay methods, it may be desirable to immobilize polypeptides or molecules to facilitate separation of complexed from uncomplexed forms of one or both of the proteins or molecules, as well as to accommodate automation of the assay.

Binding of a test agent to a target may be accomplished in any vessel suitable for containing the reactants. Non-limiting examples of such vessels include microtiter plates, test tubes, and micro-centrifuge tubes. Immobilized forms of the antibodies encompassed by the present invention may also include antibodies bound to a solid phase like a porous, microporous (with an average pore diameter less than about one micron) or macroporous (with an average pore diameter of more than about 10 microns) material, such as a membrane, cellulose, nitrocellulose, or glass fibers; a bead, such as that made of agarose or polyacrylamide or latex; or a surface of a dish, plate, or well, such as one made of polystyrene.

For example, in a direct binding assay, the polypeptides may be coupled with a radioisotope or enzymatic label such that polypeptide interactions and/or activity, such as binding events, may be determined by detecting the labeled protein in a complex. For example, the polypeptides may be labeled with ¹²⁵I, ³⁵S, ¹⁴C, or ³H, either directly or indirectly, and the radioisotope detected by direct counting of radio emmission or by scintillation counting. Alternatively, the polypeptides may be enzymatically labeled with, for example, horseradish peroxidase, alkaline phosphatase, or luciferase, and the enzymatic label detected by determination of conversion of an appropriate substrate to product.

It is also within the scope of the present invention to determine the ability of an agent to modulate a parameter of interest without the labeling of any of the interactants. For example, a microphysiometer may be used to detect interaction between polypeptides without the labeling of polypeptides to be monitored (McConnell et al. (1992) Science 257:1906-1912). As used herein, a “microphysiometer” (e.g., Cytosensor®) is an analytical instrument that measures the rate at which a cell acidifies its environment using a light-addressable potentiometric sensor (LAPS). Changes in this acidification rate may be used as an indicator of the interaction between compound and receptor.

In some embodiments, determining the ability of a test agent (e.g., antibodies, fusion proteins, peptides, or small molecules) to modulate the interaction between a given set of polypeptides may be accomplished by determining the activity of one or more members of the set of polypeptides. For example, the activity of a protein and/or one or more binding partners may be determined by detecting induction of a cellular second messenger (e.g., intracellular signaling), detecting catalytic/enzymatic activity of an appropriate substrate, detecting the induction of a reporter gene (comprising a target-responsive regulatory element operatively linked to a nucleic acid encoding a detectable marker. e.g., chloramphenicol acetyl transferase), or detecting a cellular response regulated by the protein and/or the one or more binding partners. Determining the ability of the test agent to bind to or interact with said polypeptide may be accomplished, for example, by measuring the ability of a compound to modulate immune cell costimulation or inhibition in a proliferation assay, or by interfering with the ability of said polypeptide to bind to antibodies that recognize a portion thereof.

Agents that modulate target amount and/or activity, such as interactions with one or more binding partners, may be identified by their ability to inhibit immune cell proliferation, and/or effector function, or to induce anergy, clonal deletion, and/or exhaustion when added to an in vitro assay. For example, cells may be cultured in the presence of an agent that stimulates signal transduction via an activating receptor. A number of recognized readouts of cell activation may be employed to measure, cell proliferation or effector function (e.g., antibody production, cytokine production, phagocytosis) in the presence of the agent. The ability of a test agent to block this activation may be readily determined by measuring the ability of the agent to effect a decrease in proliferation or effector function being measured, using techniques known in the art.

For example, agents encompassed by the present invention may be tested for the ability to inhibit or enhance costimulation in a T cell assay, as described in Freeman et al. (2000) J. Exp. Med, 192:1027 and Latchman et al. (2001) Nat. Immunol, 2:261, CD4+ T cells may be isolated from human PBMCs and stimulated with activating anti-CD3 antibody. Proliferation of T cells may be measured by ³H thymidine incorporation. An assay may be performed with or without CD28 costimulation in the assay. Similar assays may be performed with Jurkat T cells and PHA-blasts from PBMCs.

Alternatively, agents encompassed by the present invention may be tested for the ability to modulate cellular production of cytokines which are produced by or whose production is enhanced or inhibited in immune cells in response to modulation of the one or more biomarkers. Indicative cytokines released by immune cells of interest may be identified by ELISA or by the ability of an antibody which blocks the cytokine to inhibit immune cell proliferation or proliferation of other cell types that is induced by the cytokine, such as those described in the Examples section. An in vitro immune cell costimulation assay may also be used in a method for identifying cytokines which may be modulated by modulation of the one or more biomarkers. For example, if a particular activity induced upon costimulation, e.g., immune cell proliferation, cannot be inhibited by addition of blocking antibodies to known cytokines, the activity may result from the action of an unknown cytokine. Following costimulation, this cytokine may be purified from the media by conventional methods and its activity measured by its ability to induce immune cell proliferation. To identify cytokines which may play a role the induction of tolerance, an in vitro T cell costimulation assay as described above may be used. In this case, T cells would be given the primary activation signal and contacted with a selected cytokine, but would not be given the costimulatory signal. After washing and resting the immune cells, the cells would be rechallenged with both a primary activation signal and a costimulatory signal. If the immune cells do not respond (e.g., proliferate or produce cytokines) they have become tolerized and the cytokine has not prevented the induction of tolerance. However, if the immune cells respond, induction of tolerance has been prevented by the cytokine. Those cytokines which are capable of preventing the induction of tolerance may be targeted for blockage in vivo in conjunction with reagents which block B lymphocyte antigens as a more efficient means to induce tolerance in transplant recipients or subjects with autoimmune diseases.

In some embodiments, an assay encompassed by the present invention is a cell-free assay for screening for agents that modulate the interaction between a biomarker and/or one or more binding partners, comprising contacting a polypeptide and one or more natural binding partners, or biologically active portion thereof, with a test agent and determining the ability of the test compound to modulate the interaction between the polypeptide and one or more natural binding partners, or biologically active portion thereof. Binding of the test compound may be determined either directly or indirectly as described above. In one embodiment, the assay includes contacting the polypeptide, or biologically active portion thereof, with its binding partner to form an assay mixture, contacting the assay mixture with a test compound, and determining the ability of the test agent to interact with the polypeptide in the assay mixture, wherein determining the ability of the test agent to interact with the polypeptide comprises determining the ability of the test agent to preferentially bind to the polypeptide or biologically active portion thereof, as compared to the binding partner.

In some embodiments, whether for cell-based or cell-free assays, a test agent may further be assayed to determine whether it affects binding and/or activity of the interaction between the polypeptide and the one or more binding partners, with other binding partners. Other useful binding analysis methods include the use of real-time Biomolecular Interaction Analysis (BIA) (Sjolander and Urbaniczky (1991) Anal. Chem, 63:2338-2345 and Szabo et al. (1995) Curr. Opin. Struct. Biol, 5:699-705). As used herein, “BIA” is a technology for studying biospecific interactions in real time, without labeling any of the interactants (e.g., Biacore®). Changes in the optical phenomenon of surface plasmon resonance (SPR) may be used as an indication of real-time reactions between biological polypeptides. Polypeptides of interest may be immobilized on a Biacore® chip and multiple agents (blocking antibodies, fusion proteins, peptides, or small molecules) may be tested for binding to the polypeptide of interest. An example of using the BIA technology is described by Fitz et al. (1997) Oncogene 15:613.

The cell-free assays encompassed by the present invention are amenable to use of both soluble and/or membrane-bound forms of proteins. In the case of cell-free assays in which a membrane-bound form protein is used it may be desirable to utilize a solubilizing agent such that the membrane-bound form of the protein is maintained in solution. Examples of such solubilizing agents include non-ionic detergents such as n-octylglucoside, n-dodecylglucoside, n-dodecylmaltoside, octanoyl-N-methylglucamide, decanoyl-N-methylglucamide, Triton® X-100, Triton® X-114. Thesit®, Isotridecypoly(ethylene glycol ether)ₙ, 3-[(3-cholamidopropyl)dimethylamminio]-1-propane sulfonate (CHAPS), 3-[(3-cholamidopropyl)dimethylammonio]-2-hydroxy-1-propane sulfonate (CHAPSO), or N-dodecyl=N,N-dimethyl-3-ammonio-1-propane sulfonate.

In one or more embodiments of the above described assay methods, it may be desirable to immobilize either polypeptides to facilitate separation of complexed from uncomplexed forms of one or both of the proteins, as well as to accommodate automation of the assay. Binding of a test compound to a polypeptide, may be accomplished in any vessel suitable for containing the reactants. Examples of such vessels include microtiter plates, test tubes, and micro-centrifuge tubes. In one embodiment, a fusion protein may be provided which adds a domain that allows one or both of the proteins to be bound to a matrix. For example, glutathione-S-transferase-based polypeptide fusion proteins, or glutathione-S-transferase/target fusion proteins, may be adsorbed onto glutathione sepharose beads (Sigma Chemical, St. Louis, MO) or glutathione derivatized microtiter plates, which are then combined with the test compound, and the mixture incubated under conditions conducive to complex formation (e.g., at physiological conditions for salt and pH). Following incubation, the beads or microtiter plate wells are washed to remove any unbound components, the matrix immobilized in the case of beads, complex determined either directly or indirectly, for example, as described above. Alternatively, the complexes may be dissociated from the matrix, and the level of polypeptide binding or activity determined using standard techniques.

The present invention further pertains to novel agents identified by the above-described screening assays. Accordingly, it is within the scope of the present invention to further use an agent identified as described herein in an appropriate model system. For example, an agent identified as described herein may be used in a model system to determine the efficacy, toxicity, or side effects of treatment with such an agent. Alternatively, an agent identified as described herein may be used in a model system to determine the mechanism of action of such an agent. Furthermore, the present invention pertains to uses of novel agents identified by the above-described screening assays for treatments as described herein.

d. Predictive Medicine

The present invention also pertains to the field of predictive medicine in which diagnostic assays, prognostic assays, and monitoring clinical trials are used for prognostic (predictive) purposes to thereby treat an individual prophylactically. Accordingly, one aspect encompassed by the present invention encompasses diagnostic assays for determining (e.g., detecting) the presence, absence, amount, and/or activity level of PRAME or reactivity to PRAME in the context of a biological sample (e.g., blood, serum, cells, or tissue) to thereby determine whether an individual afflicted with a disorder characterized by PRAME expression is likely to respond to therapy, whether in an original state or as a recurrence. Such assays may be used for prognostic or predictive purpose to thereby prophylactically treat an individual prior to the onset or after recurrence of a disorder characterized by PRAME expression.

The diagnostic methods described herein may furthermore be utilized to identify subjects having or at risk of developing a disorder associated with expression or lack thereof of PRAME. As used herein, the term “aberrant” includes an upregulation or downregulation of PRAME from normal levels. Aberrant expression or activity includes increased or decreased expression or activity, as well as expression or activity which does not follow the normal developmental pattern of expression or the subcellular pattern of expression. For example, aberrant levels is intended to include the cases in which a mutation in the biomarker gene or regulatory sequence, or amplification of the chromosomal gene, thereof causes upregulation or downregulation of the biomarker of interest. As used herein, the term “unwanted” includes an unwanted phenomenon involved in a biological response, such as immune cell activity.

The assays described herein, such as the preceding diagnostic assays or the following assays, may be utilized to identify a subject having or at risk of developing a disorder associated with PRAME misregulation. Thus, the present invention provides a method for identifying a disorder associated with aberrant or unwanted PRAME regulation in which a test sample is obtained from a subject and PRAME expression is detected, wherein the presence of PRAME expression is diagnostic for a subject having or at risk of developing the disorder associated with aberrant or unwanted PRAME expression. As used herein, a “test sample” refers to a biological sample obtained from a subject of interest. For example, a test sample may be a biological fluid (e.g., cerebrospinal fluid or serum), cell sample, or tissue, such as a histopathological slide of the tumor microenvironment, peritumoral area, and/or intratumoral area.

Furthermore, the prognostic assays described herein may be used to determine whether a subject may be administered an agent described herein to treat such a disorder associated with aberrant or unwanted PRAME expression. For example, such methods may be used to determine whether a subject may be effectively treated with one or a combination of agents. Thus, the present invention provides methods for determining whether a subject may be effectively treated with one or more agents described herein for treating a disorder associated with aberrant or unwanted PRAME expression.

The methods described herein may be performed, for example, by utilizing pre-packaged diagnostic kits comprising at least one antibody reagent described herein, which may be conveniently used, e.g., in clinical settings to diagnose patients exhibiting symptoms or family history of a disease or illness involving the biomarker of interest.

Furthermore, any cell type or tissue in which the biomarker of interest is expressed may be utilized in the prognostic assays described herein.

e. Monitoring of Effects During Clinical Trials

Monitoring the influence of a disorder characterized by PRAME expression therapy (e.g., compounds, drugs, vaccines, cell therapies, and the like) on immune responses, such as T cell reactivity (e.g., the presence of binding and/or T cell activation and/or effector function), may be applied not only in basic candidate PRAME antigen binding molecule screening, but also in clinical trials. For example, the effectiveness of immunogenic peptides, pMHCs, engineered cells, binding proteins, and related compostions described herein to increase an immune response (e.g., T cell immune response) against cells of interest, such as hyperproliferative cells, expressing PRAME, may be monitored in clinical trials of subjects afflicted with a disorder characterized by PRAME expression. In such clinical trials, the presence of binding and/or T cell activation and/or effector function (e.g., T cell proliferation, killing, and/or cytokine release), may be used as a “read out” or marker of the phenotype of a particular cell, tissue, or system. Similarly, the effectiveness of an adaptive T cell therapy with T cells engineered to express a binding protein (e.g., a TCR, an antigen-binding fragment of a TCR, a CAR, or a fusion protein comprising a TCR and an effector domain) as described herein to increase immune response to cells of interest, such as hyperproliferative cells, that are expressing PRAME, may be monitored in clinical trials of subjects having a disorder characterized by PRAME expression. In such clinical trials, the presence of binding and/or T cell activation and/or effector function (e.g., T cell proliferation, killing, or cytokine release), may be used as a “read out” or marker of the phenotype of a particular cell, tissue, or system.

In some embodiments, the present invention provides a method for monitoring the effectiveness of treatment of a therapy (e.g., compounds, drugs, vaccines, cell therapies, and the like) including the steps of a) determining the absence, presence, or level of reactivity between a sample obtained from the subject and one or more binding proteins or related composition, in a first sample obtained from the subject prior to providing at least a portion of the therapy for the disorder characterized by PRAME expression to the subject, and b) determining the absence, presence, or level of reactivity between the one or more binding proteins or related composition, and a sample obtained from the subject present in a second sample obtained from the subject following provision of the portion of the therapy, wherein the presence or a higher level of reactivity in the first sample, relative to the second sample, is an indication that the therapy is efficacious for treating the disorder characterized by PRAME expression in the subject and wherein the absence or a lower level of reactivity in the first sample, relative to the second sample, is an indication that the therapy is not efficacious for treating the disorder characterized by PRAME expression in the subject.

In some embodiments, the present invention provides a method for monitoring the effectiveness of treatment of a subject with an agent (e.g., antibodies, an agonist, antagonist, peptidomimetic, polypeptide, peptide, nucleic acid, small molecule, or other drug candidate identified by the screening assays described herein) including the steps of (i) obtaining a preadministration sample from a subject prior to administration of the agent; (ii) detecting PRAME expression in the preadministration sample; (iii) obtaining one or more post-administration samples from the subject; (iv) detecting PRAME expression in the post-administration samples; (v) comparing the PRAME expression in the preadministration sample with the PRAME expression in the post-administration sample; and (vi) altering the administration of the agent to the subject accordingly. Biomarker polypeptide analysis, such as by immunohistochemistry (IHC), may also be used to select patients who will receive therapy, such as immunotherapy.

In addition, the prognostic methods described herein may be used to determine whether a subject may be administered a therapeutic agent to treat a disorder associated with PRAME expression.

f. Clinical Efficacy

Clinical efficacy may be measured by any method known in the art. For example, the response to a therapy relates to any response of the disorder associated with PRAME expression, e.g., a tumor, to the therapy, preferably to a change in the number of cancer cells, tumor mass, and/or tumor volume, such as after initiation of neoadjuvant or adjuvant chemotherapy. Tumor response may be assessed in a neoadjuvant or adjuvant situation where the size of a tumor after systemic intervention may be compared to the initial size and dimensions as measured by CT, PET, mammogram, ultrasound or palpation and the cellularity of a tumor may be estimated histologically and compared to the cellularity of a tumor biopsy taken before initiation of treatment. Response may also be assessed by caliper measurement or pathological examination of the tumor after biopsy or surgical resection. Response may be recorded in a quantitative fashion such as percentage change in tumor volume or cellularity or by using a semi-quantitative scoring system such as residual cancer burden (Symmans et al. (2007) J. Clin. Oncol, 25:4414-4422) or Miller-Payne score (Ogston et al. (2003) Breast (Edinburgh, Scotland) 12:320-327) in a qualitative fashion like “pathological complete response” (pCR), “clinical complete remission” (cCR), “clinical partial remission” (cPR), “clinical stable disease” (cSD). “clinical progressive disease” (cPD) or other qualitative criteria. Assessment of tumor response may be performed early after the onset of neoadjuvant or adjuvant therapy (e.g., after a few hours, days, weeks or preferably after a few months). A typical endpoint for response assessment is upon termination of neoadjuvant chemotherapy or upon surgical removal of residual tumor cells and/or the tumor bed.

In some embodiments, clinical efficacy of the therapeutic treatments described herein may be determined by measuring the clinical benefit rate (CBR). The clinical benefit rate is measured by determining the sum of the percentage of patients who are in complete remission (CR), the number of patients who are in partial remission (PR) and the number of patients having stable disease (SD) at a time point at least 6 months out from the end of therapy. The shorthand for this formula is CBR=CR+PR+SD over 6 months. In some embodiments, the CBR for a particular modulator of biomarkers listed in Table 1 therapeutic regimen is at least 25%, 30%, 35%, 40%, 45%, 50%, 55%, 60%, 65%, 70%, 75%, 80%, 85%, or more.

Additional criteria for evaluating the response to cancer therapy are related to “survival,” which includes all of the following: survival until mortality, also known as overall survival (wherein said mortality may be either irrespective of cause or tumor related); “recurrence-free survival” (wherein the term recurrence shall include both localized and distant recurrence); metastasis free survival; disease free survival (wherein the term disease shall include cancer and diseases associated therewith). The length of said survival may be calculated by reference to a defined start point (e.g., time of diagnosis or start of treatment) and end point (e.g., death, recurrence, or metastasis). In addition, criteria for efficacy of treatment may be expanded to include response to chemotherapy, probability of survival, probability of metastasis within a given time period, and probability of tumor recurrence.

For example, in order to determine appropriate threshold values, a particular agent of interest may be administered to a population of subjects and the outcome may be correlated to biomarker measurements that were determined prior to administration of any therapy. The outcome measurement may be pathologic response to therapy given in the neoadjuvant setting. Alternatively, outcome measures, such as overall survival and disease-free survival may be monitored over a period of time for subjects following therapy for whom PRAME expression values are known. In certain embodiments, the same doses of the agent are administered to each subject. The period of time for which subjects are monitored may vary. For example, subjects may be monitored for at least 1, 2, 3, 4, 5, 6, 7, 8, 9, 10, 11, 12, 14, 16, 18, 20, 25, 30, 35, 40, 45, 50, 55, 60 months, or longer. PRAME measurement threshold values that correlate to outcome of a therapy may be determined using well-known methods, such as those described in the Examples section.

### VII. Cell Therapy

In another aspect encompassed by the present invention, the methods include adoptive cell therapy, whereby genetically engineered cells expressing the provided molecules targeting an MHC-restricted epitope (e.g., cells expressing a binding protein (e.g., a TCR or CAR) or antigen-binding fragment thereof) are administered to subjects. Such administration may promote activation of immune cells (e.g., T cell activation) in an antigen-targeted manner, such that the cells of interest such as hyperproliferative cells, express a PRAME antigen are targeted for destruction.

Thus, the provided methods and uses include methods and uses for adoptive cell therapy. In some embodiments, the methods include administration of the cells or a composition containing the cells to a subject, tissue, or cell, such as one having, at risk for, or suspected of having the disease, condition or disorder. In some embodiments, the cells, populations, and compositions are administered to a subject having the particular disease or condition to be treated (e.g., via adoptive cell therapy, such as by adoptive T cell therapy). In some embodiments, the cells or compositions are administered to the subject, such as a subject having or at risk for the disease or condition. In some embodiments, the methods thereby treat, e.g., ameliorate one or more symptom of the disease or condition.

Methods for administration of cells for adoptive cell therapy are known and may be used in connection with the provided methods and compositions (e.g., U.S. Pat. Publ. No. 2003/0170238. U.S. Pat. No. 4,690,915. Rosenberg (2011) Nat. Rev. Clin. Oncol. 8:577-585. Themeli et al. (2013) Nat. Biotechnol, 31:928-933, Tsukahara et al. (2013) Biochem. Biophys. Res. Commun, 438:84-89, and Davila et al. (2013) PLoS ONE 8:e61338).

In some embodiments, cell therapy (e.g., adoptive cell therapy, such as adoptive T cell therapy) may be carried out by autologous transfer, in which the cells are isolated and/or otherwise prepared from the subject who is to receive the cell therapy, or from a sample derived from such a subject. Thus, in some embodiments, the cells are derived from a subject. e.g., patient, in need of a treatment and the cells, following isolation and processing are administered to the same subject.

In some embodiments, the cell therapy (e.g., adoptive cell therapy, such as adoptive T cell therapy) may be carried out by allogeneic transfer, in which the cells are isolated and/or otherwise prepared from a subject other than a subject who is to receive or who ultimately receives the cell therapy, e.g., a first subject. In such embodiments, the cells then are administered to a different subject, e.g., a second subject, of the same species. In some embodiments, the first and second subjects are genetically identical (syngeneic). In some embodiments, the first and second subjects are genetically similar. In some embodiments, the second subject expresses the same HLA class or supertype as the first subject.

In some embodiments, the subject, to whom the cells, cell populations, or compositions are administered is a primate, such as a human. In some embodiments, the primate is a monkey or an ape. The subject may be male or female and may be any suitable age, including infant, juvenile, adolescent, adult, and geriatric subjects. In some embodiments, the subject is a non-primate mammal, such as a rodent. In some examples, the patient or subject is a validated animal model for disease, adoptive cell therapy, and/or for assessing toxic outcomes such as cytokine release syndrome (CRS).

The binding molecules, such as TCRs, antigen-binding fragments of TCRs (e.g., scTCRs) and chimeric receptors (e.g., CARs) containing the TCR, and cells expressing the same, may be administered by any suitable means, for example, by injection, e.g., intravenous or subcutaneous injections, intraocular injection, periocular injection, subretinal injection, intravitreal injection, trans-septal injection, subscleral injection, intrachoroidal injection, intracameral injection, subconjunctival injection, subconjunctival injection, sub-Tenon's injection, retrobulbar injection, peribulbar injection, or posterior juxtascleral delivery. In some embodiments, they are administered by parenteral, intrapulmonary, and intranasal, and, if desired for local treatment, intralesional administration. Parenteral infusions include intramuscular, intravenous, intraarterial, intraperitoneal, or subcutaneous administration. Dosing and administration may depend in part on whether the administration is brief or chronic. Various dosing schedules include but are not limited to single or multiple administrations over various time-points, bolus administration, and pulse infusion.

For the prevention or treatment of disease, the appropriate dosage of the binding molecule or cell may depend on the type of disease to be treated, the type of binding molecule, the severity and course of the disease, whether the binding molecule is administered for preventive or therapeutic purposes, previous therapy, the patient's clinical history and response to the binding molecule, and the discretion of the attending physician. The compositions and molecules and cells are in some embodiments suitably administered to the patient at one time or over a series of treatments.

In some embodiments, cells may be administered at 0.1×10⁶, 0.2×10⁶, 0.3×10⁶, 0.4×10⁶, 0.5×10⁶, 0.6×10⁶. 0.7×10⁶, 0.8×10⁶, 0.9×10⁶, 1.0×10⁶, 5.0×10⁶, 1.0×10⁷, 5.0×10⁷, 1.0×10¹. 5.0×10⁸, or more, or any range in between or any value in between, cells per kilogram of subject body weight. The number of cells transplanted may be adjusted based on the desired level of engraftment in a given amount of time. Generally, 1×10⁵ to about 1×10⁹ cells/kg of body weight, from about 1×10⁶ to about 1×10⁸ cells/kg of body weight, or about 1×10⁷ cells/kg of body weight, or more cells, as necessary, may be transplanted. In some embodiment, transplantation of at least about 0.1×10⁶. 0.5×10⁶, 1.0×10⁶, 2.0×10⁶. 3.0×10⁶, 4.0×10⁶, or 5.0×10⁶ total cells relative to an average size mouse is effective. For example, in some embodiments, cells, or individual populations of sub-types of cells, may be administered to the subject at a range of about one million to about 100 billion cells and/or that amount of cells per kilogram of body weight, such as, e.g., 1 million to about 50 billion cells (e.g., about 5 million cells, about 25 million cells, about 500 million cells, about 1 billion cells, about 5 billion cells, about 20 billion cells, about 30 billion cells, about 40 billion cells, or a range defined by any two of the foregoing values), such as about 10 million to about 100 billion cells (e.g., about 20 million cells, about 30 million cells, about 40 million cells, about 60 million cells, about 70 million cells, about 80 million cells, about 90 million cells, about 10 billion cells, about 25 billion cells, about 50 billion cells, about 75 billion cells, about 90 billion cells, or a range defined by any two of the foregoing values), and in some cases about 100 million cells to about 50 billion cells (e.g., about 120 million cells, about 250 million cells, about 350 million cells, about 450 million cells, about 650 million cells, about 800 million cells, about 900 million cells, about 3 billion cells, about 30 billion cells, about 45 billion cells) or any value in between these ranges and/or per kilogram of body weight. Dosages may vary depending on attributes particular to the disease or disorder and/or patient and/or other treatments.

Engraftment of transplanted cells may be assessed by any of various methods, such as, but not limited to, tumor volume, cytokine levels, time of administration, flow cytometric analysis of cells of interest obtained from the subject at one or more time points following transplantation, and the like. For example, a time-based analysis of waiting 1, 2, 3, 4, 5, 6, 7, 8, 9, 10, 11, 12, 13, 14, 15, 16, 17, 18, 19, 20, 21, 22, 23, 24, 25, 26, 27, 28 days or may signal the time for tumor harvesting (e.g., providing a dose of TCR-T cells infused 28 days apart). Any such metrics are variables that may be adjusted according to well-known parameters in order to determine the effect of the variable on a response to anti-cancer immunotherapy. In addition, the transplanted cells may be co-transplanted with other agents, such as cytokines, extracellular matrices, cell culture supports, and the like.

Cells may also be administered before, concurrently with, or after, other anti-cancer agents.

Two or more cell types may be combined and administered, such as cell-based therapy and adoptive cell transfer of stem cells, cancer vaccines and cell-based therapy, and the like. For example, adoptive cell-based immunotherapies may be combined with the cell-based therapies encompassed by the present invention. In some embodiments, the cell-based agents may be used alone or in combination with additional cell-based agents, such as immunotherapies like adoptive T cell therapy (ACT). For example, T cells genetically engineered to recognize CD19 used to treat follicular B cell lymphoma. Immune cells for ACT may be dendritic cells. T cells such as CD8⁺ T cells and CD4⁺ T cells, natural killer (NK) cells, NK T cells, cytotoxic T lymphocytes (CTLs), tumor infiltrating lymphocytes (TILs), lymphokine activated killer (LAK) cells, memory T cells, regulatory T cells (Tregs), helper T cells, cytokine-induced killer (CIK) cells, and any combination thereof. Well-known adoptive cell-based immunotherapeutic modalities, including, without limitation, irradiated autologous or allogeneic tumor cells, tumor lysates or apoptotic tumor cells, antigen-presenting cell-based immunotherapy, dendritic cell-based immunotherapy, adoptive T cell transfer, adoptive CAR T cell therapy, autologous immune enhancement therapy (AIET), cancer vaccines, and/or antigen presenting cells. Such cell-based immunotherapies may be further modified to express one or more gene products to further modulate immune responses, such as expressing cytokines like GM-CSF, and/or to express tumor-associated antigen (TAA) antigens, such as Mage-1, gp-100, and the like. The ratio of an agent encompassed by the present invention, such as cancer cells, to another agent encompassed by the present invention or other composition may be 1:1 relative to each other (e.g., equal amounts of 2 agents, 3 agents, 4 agents, etc.), but may modulated in any amount desired (e.g., 1:1, 1.1:1, 1.2:1, 1.3:1, 1.4:1, 1.5:1, 2:1, 2.5:1, 3:1, 3.5:1, 4:1, 4.5:1, 5:1, 5.5:1, 6:1, 6.5:1, 7:1, 7.5:1, 8:1, 8.5:1, 9:1, 9.5:1, 10:1, or greater).

In some embodiments, for example, where the subject is a human, the dose includes fewer than about 1×10⁸ total binding protein (e.g., TCR or CAR)-expressing cells, T cells, or peripheral blood mononuclear cells (PBMCs), e.g., in the range of about 1×10⁶ to 1×10⁸ such cells, such as 2×10⁶, 5×10⁶, 1×10⁷, 5×10⁷, or 1×10⁸ or total such cells, or the range between any two of the foregoing values.

In some embodiments, the cells or related compositions described herein, such as nucleic acids, host cells, pharmaceutical formulations, and the like, may be administered as part of a combination treatment, such as simultaneously with or sequentially with, in any order, another therapeutic intervention, such as another antibody or engineered cell or receptor or agent, such as a cytotoxic or therapeutic agent.

In some embodiments, the cells or related composition may be co-administered with one or more additional therapeutic agents or in connection with another therapeutic intervention, either simultaneously or sequentially in any order. In some contexts, the cells or related composition are co-administered with another therapy sufficiently close in time such that the cell populations enhance the effect of one or more additional therapeutic agents, or vice versa. In some embodiments, the cells or related composition are administered prior to the one or more additional therapeutic agents. In some embodiments, the cells or related composition are administered after to the one or more additional therapeutic agents.

In some embodiments, the biological activity of the cells or related composition is measured by any of a number of known methods once the cells or related composition are administered to a subject (e.g., a human). Parameters to assess include specific binding of an engineered or natural T cell or other immune cell to antigen, in vivo, e.g., by imaging, or in vitro/ex vivo, e.g., by ELISA or flow cytometry. In some embodiments, the ability of the cells to destroy target cells (cytotoxicity) may be measured using any suitable assay or method known in the art (e.g., Kochenderfer et al. (2009) J. Immunother, 32: 689-702 and Herman et al. (2004) J. Immunol. Meth, 285:25-40). In some embodiments, the biological activity of the cells also may be measured by assaying expression and/or secretion of certain cytokines, such as CD107a, IFNγ, IL-2, and TNF alpha. In some embodiments, the biological activity is measured by assessing clinical outcome, such as reduction in viral burden or load.

In some embodiments, cells are modified in any number of ways, such that their therapeutic or prophylactic efficacy is increased. For example, the binding protein (e.g., engineered TCR. CAR, or antigen-binding fragment thereof) expressed by the population may be conjugated either directly or indirectly through a linker to a targeting moiety. The practice of conjugating compounds to targeting moieties is well-known in the art (e.g., Wadwa et al. (1995) J. Drug Targeting 3:111 and U.S. Pat. No. 5,087,616).

Immune cells, such as cytotoxic lymphocytes, may be obtained from any suitable source such as peripheral blood, spleen, and lymph nodes. The immune cells may be used as crude preparations or as partially purified or substantially purified preparations, which may be obtained by standard techniques, including, but not limited to, methods involving immunomagnetic or flow cytometry techniques using antibodies.

In certain aspects, the PRAME immunogenic peptides described herein, or a nucleic acid encoding such PRAME immunogenic peptides, may be used in compositions and methods for providing PRAME-primed, antigen-presenting cells, and/or PRAME-specific lymphocytes generated with these antigen-presenting cells. In some embodiments, such antigen-presenting cells and/or lymphocytes are used in the treatment and/or prevention of a disorder associated with PRAME expression.

In some aspects, provided herein are methods for making PRAME-primed, antigen-presenting cells by contacting antigen-presenting cells with a PRAME immunogenic peptide described herein, or nucleic acids encoding the at least one PRAME immunogenic peptide, alone or in combination with an adjuvant, in vitro under a condition sufficient for the at least one PRAME immunogenic polypeptide to be presented by the antigen-presenting cells.

In some embodiments. PRAME immunogenic polypeptide, or nucleic acid encoding the PRAME immunogenic polypeptide, alone or in combination with an adjuvant, may be contacted with a homogenous, substantially homogenous, or heterogeneous composition comprising antigen-presenting cells. For example, the composition may include but is not limited to whole blood, fresh blood, or fractions thereof such as, but not limited to, peripheral blood mononuclear cells, buffy coat fractions of whole blood, packed red cells, irradiated blood, dendritic cells, monocytes, macrophages, neutrophils, lymphocytes, natural killer cells, and natural killer T cells. If, optionally, precursors of antigen-presenting cells are used, the precursors may be cultured under suitable culture conditions sufficient to differentiate the precursors into antigen-presenting cells. In some embodiments, the antigen-presenting cells (or precursors thereof) are selected from monocytes, macrophages, cells of myeloid lineage, B cells, dendritic cells, or Langerhans cells.

The amount of the PRAME immunogenic polypeptide, or nucleic acid encoding the PRAME immunogenic polypeptide, alone or in combination with an adjuvant, to be placed in contact with antigen-presenting cells may be determined by one of ordinary skill in the art by routine experimentation. Generally, antigen-presenting cells are contacted with the PRAME immunogenic polypeptide, or nucleic acid encoding the PRAME immunogenic polypeptide, alone or in combination with an adjuvant, for a period of time sufficient for cells to present the processed forms of the antigens for the modulation of T cells. In one embodiment, antigen-presenting cells are incubated in the presence of the PRAME immunogenic polypeptide, or nucleic acid encoding the PRAME immunogenic polypeptide, alone or in combination with an adjuvant, for less than about a week, illustratively, for about 1 minute to about 48 hours, about 2 minutes to about 36 hours, about 3 minutes to about 24 hours, about 4 minutes to about 12 hours, about 6 minutes to about 8 hours, about 8 minutes to about 6 hours, about 10 minutes to about 5 hours, about 15 minutes to about 4 hours, about 20 minutes to about 3 hours, about 30 minutes to about 2 hours, and about 40 minutes to about 1 hour. The time and amount of the PRAME immunogenic polypeptide, or nucleic acid encoding the PRAME immunogenic polypeptide, alone or in combination with an adjuvant, necessary for the antigen presenting cells to process and present the antigens may be determined, for example using pulse-chase methods wherein contact is followed by a washout period and exposure to a read-out system e.g., antigen reactive T cells.

In certain embodiments, any appropriate method for delivery of antigens to the endogenous processing pathway of the antigen-presenting cells may be used. Such methods include but are not limited to, methods involving pH-sensitive liposomes, coupling of antigens to adjuvants, apoptotic cell delivery, pulsing cells onto dendritic cells, delivering recombinant chimeric virus-like particles (VLPs) comprising antigen to the MHC class I processing pathway of a dendritic cell line.

In one embodiment, solubilized PRAME immunogenic polypeptide is incubated with antigen-presenting cells. In some embodiments, the PRAME immunogenic polypeptide may be coupled to a cytolysin to enhance the transfer of the antigens into the cytosol of an antigen-presenting cell for delivery to the MHC class I pathway. Exemplary cytolysins include saponin compounds such as saponin-containing Immune Stimulating Complexes (ISCOM5), pore-forming toxins (e.g., an alpha-toxin), and natural cytolysins of gram-positive bacteria such as listeriolysin O (LLO), streptolysin O (SLO), and perfringolysin O (PFO).

In some embodiments, antigen-presenting cells, such as dendritic cells and macrophage, may be isolated according to methods known in the art and transfected with polynucleotides by methods known in the art for introducing a nucleic acid encoding the PRAME immunogenic polypeptide into the antigen-presenting cell. Transfection reagents and methods are known in the art and commercially available. For example, RNA encoding PRAME immunogenic polypeptide may be provided in a suitable medium and combined with a lipid (e.g., a cationic lipid) prior to contact with antigen-presenting cells. Non-limiting examples of such lipids include LIPOFECTIN™ and LIPOFECTAMINE™. The resulting polynucleotide-lipid complex may then be contacted with antigen-presenting cells. Alternatively, the polynucleotide may be introduced into antigen-presenting cells using techniques such as electroporation or calcium phosphate transfection. The polynucleotide-loaded antigen-presenting cells may then be used to stimulate T lymphocyte (e.g., cytotoxic T lymphocyte) proliferation in vitro, ex vivo, or in vivo. In one embodiment, the ex vivo expanded T lymphocyte is administered to a subject in a method of adoptive immunotherapy.

In certain aspects, provided herein is a composition comprising antigen-presenting cells that have been contacted in vitro with a PRAME immunogenic polypeptide, or a nucleic acid encoding a PRAME immunogenic polypeptide, alone or in combination with an adjuvant under a condition sufficient for a PRAME immunogenic epitope to be presented by the antigen-presenting cells.

In some aspects, provided herein is a method for preparing lymphocytes specific for a PRAME protein. The method comprises contacting lymphocytes with the antigen-presenting cells described above under conditions sufficient to produce a PRAME protein-specific lymphocyte capable of eliciting an immune response against a cell that is infected by the PRAME virus. Thus, the antigen-presenting cells also may be used to provide lymphocytes, including T lymphocytes and B lymphocytes, for eliciting an immune response against cell that is infected by the PRAME virus.

In some embodiments, a preparation of T lymphocytes is contacted with the antigen-presenting cells described above for a period of time, (e.g., at least about 24 hours) to priming the T lymphocytes to a PRAME immunogenic epitope presented by the antigen-presenting cells.

In some embodiments, a population of antigen-presenting cells may be co-cultured with a heterogeneous population of peripheral blood T lymphocytes together with a PRAME immunogenic polypeptide, or a nucleic acid encoding a PRAME immunogenic polypeptide, alone or in combination with an adjuvant. The cells may be co-cultured for a period of time and under conditions sufficient for PRAME epitopes included in the PRAME polypeptides to be presented by the antigen-presenting cells and the antigen-presenting cells to prime a population of T lymphocytes to respond to cells is infected by the PRAME virus. In certain embodiments, provided herein are T lymphocytes and B lymphocytes that are primed to respond to cells that is infected by the PRAME virus.

T lymphocytes may be obtained from any suitable source such as peripheral blood, spleen, and lymph nodes. The T lymphocytes may be used as crude preparations or as partially purified or substantially purified preparations, which may be obtained by standard techniques including, but not limited to, methods involving immunomagnetic or flow cytometry techniques using antibodies.

In certain aspects, provided herein is a composition (e.g., a pharmaceutical composition) comprising the antigen-presenting cells or the lymphocytes described above, and a pharmaceutically acceptable carrier and/or diluent. In some embodiments, the composition further comprises an adjuvant as described above.

In certain aspects and as further described above, provided herein is a method for eliciting an immune response to the cell is infected by the PRAME virus, the method comprising administering to the subject the antigen-presenting cells or the lymphocytes described above in effective amounts sufficient to elicit the immune response. In some embodiments, provided herein is a method for treatment or prophylaxis of a disorder characterized by PRAME expression, the method comprising administering to the subject an effective amount of the antigen-presenting cells or the lymphocytes described above. In one embodiment, the antigen-presenting cells or the lymphocytes are administered systemically, preferably by injection. Alternately, one may administer locally rather than systemically, for example, via injection directly into tissue, preferably in a depot or sustained release formulation.

In certain embodiments, the antigen-primed antigen-presenting cells described herein and the antigen-specific T lymphocytes generated with these antigen-presenting cells may be used as active compounds in immunomodulating compositions for prophylactic or therapeutic treatment of a disorder characterized by PRAME expression. In some embodiments, the PRAME-primed antigen-presenting cells described herein may be used for generating CD8⁺ T lymphocytes, CD4⁺ T lymphocytes, and/or B lymphocytes for adoptive transfer to the subject. Thus, for example, PRAME-specific lymphocyte may be adoptively transferred for therapeutic purposes in subjects afflicted with a disorder characterized by PRAME expression.

In certain embodiments, the antigen-presenting cells and/or lymphocytes described herein may be administered to a subject, either by themselves or in combination, for eliciting an immune response, particularly for eliciting an immune response to cells expressing PRAME. In some embodiments, the antigen-presenting cells and/or lymphocytes may be derived from the subject (i.e., autologous cells) or from a different subject that is MHC matched or mismatched with the subject (e.g., allogeneic).

Single or multiple administrations of the antigen-presenting cells and lymphocytes may be carried out with cell numbers and treatment being selected by the care provider (e.g., physician). In some embodiments, the antigen-presenting cells and/or lymphocytes are administered in a pharmaceutically acceptable carrier. Suitable carriers may be growth medium in which the cells were grown, or any suitable buffering medium such as phosphate buffered saline. The cells may be administered alone or as an adjunct therapy in conjunction with other therapeutics.

In another aspect encompassed by the present invention, provided herein is a method for eliciting an immune response to a cell that expresses a PRAME antigen, the method comprising administering to the subject cells described herein expressing a binding protein (e.g., engineered TCR. CAR, or antigen-binding fragment thereof) in effective amounts sufficient to elicit the immune response. In some embodiments, provided herein is a method for treatment or prophylaxis of a disorder characterized by PRAME expression (e.g., a non-malignant disorder, a hyperproliferative disorder, or a relapse of a hyperproliferative disorder characterized by PRAME expression), the method comprising administering to the subject an effective amount of the cells described herein expressing a binding protein (e.g., engineered TCR, CAR, or antigen-binding fragment thereof). In one embodiment, the cells are administered systemically, such as by injection. Alternately, one may administer locally rather than systemically, for example, via injection directly into tissue, such as in a depot or sustained release formulation.

In some embodiments, the cells described herein expressing a binding protein (e.g., engineered TCR, CAR, or antigen-binding fragment thereof) may be used as active compounds in immunomodulating compositions for prophylactic or therapeutic treatment of a disorder characterized by PRAME expression (e.g., a non-malignant disorder, a hyperproliferative disorder, or a relapse of a hyperproliferative disorder characterized by PRAME expression). In some embodiments, PRAME-primed antigen-presenting cells may be used for generating lymphocytes (e.g., CD8⁺ T lymphocytes, CD4+ T lymphocytes, and/or B lymphocytes), for further use in adoptive transfer to the subject with the cells described herein expressing a binding protein (e.g., engineered TCR, CAR, or antigen-binding fragment thereof).

In some embodiments, the cells described herein expressing a binding protein (e.g., engineered TCR. CAR, or antigen-binding fragment thereof), either alone or in combination with the lymphocytes, may be administered to a subject for eliciting an immune response, particularly for eliciting an immune response to cells are expressing a PRAME antigen.

As described above, single or multiple administrations of the cells described herein expressing a binding protein (e.g., engineered TCR, CAR, or antigen-binding fragment thereof) cells, either alone or in combination with the lymphocytes, may be carried out with cell numbers and treatment being selected by the care provider (e.g., physician). Similarly, the cells, either alone or in combination with lymphocytes, may be administered in a pharmaceutically acceptable carrier. Suitable carriers may be growth medium in which the cells were grown, or any suitable buffering medium such as phosphate buffered saline. Cells may be administered alone or as an adjunct therapy in conjunction with other therapeutics.

### VIII. Kits and Devices

The present invention also encompasses kits and devices. For example, the kit or device may comprise binding proteins, nucleic acids or vectors comprising sequences encoding binding proteins, host cells comprising nucleic acids or vectors and/or expressing the binding proteins as described herein, stable MHC-peptide complexes, adjuvants, detection reagents, and combinations thereof, packaged in a suitable container and may further comprise instructions for using such reagents. The kit or device may also contain other components, such as administration tools packaged in a separate container. The kit or device may be promoted, distributed, or sold as a unit for performing the methods encompassed by the present invention.

The disclosure is further illustrated by the following examples which should not be construed as limiting.

### EXAMPLES

### Example 1: Materials and Methods for Example 2

a. Human Peripheral Blood Mononuclear Cell Collection

HLA-A*02:01-positive healthy donor leukopaks were collected by HemaCare (Los Angeles, CA), StemExpress (Placerville, CA), and Discovery Life Sciences (Huntsville, AL) using their IRB-approved protocols. Peripheral blood mononuclear cells (PBMCs) were isolated from fresh leukopaks from HemaCare and Discovery Life Sciences by density gradient centrifugation using Lymphocyte Separation Medium (Corning, Corning. NY). PBMCs contained in the lymphocyte layer were collected following centrifugation, washed three times with DPBS (Cytiva, Marlborough. MA), and counted. PBMCs were isolated from StemExpress leukopaks either by density gradient centrifugation as above, or using a Custom Leukopak PBMC Isolation kit (Miltenyi Biotec, Auburn, CA) on the MultiMACS™ Cell24 Separator Plus instrument, version 3 (Miltenyi Biotec) per the manufacturer's instructions. Isolated PBMCs were frozen in CryoStor® CS10 (StemCell Technologies, Cambridge, MA) and stored in liquid nitrogen.

b. ReceptorScan Screens

### (i) DC Culture

Monocyte isolation was performed on day −4 using PBMCs isolated from HLA-A*02:01-positive healthy donors with the EasySep™ Human CD14 Positive Selection Kit II (StemCell Technologies) according to the manufacturer's instructions. Purity and costimulatory molecule expression were assessed using fluorescently-labeled antibodies specific for CD14 (M5 E2. BioLegend, Dedham, MA), HLA-A2 (BB7.2, BioLegend), CD80 (2D10, BioLegend), CD83 (HB15e, BioLegend), and CD86 (1T2.2, Biolegend); CD14 expression was >90%. CD14⁺ monocytes were resuspended in AIM-V media (Thermo Fisher Scientific, Waltham, MA) supplemented with recombinant human GM-CSF and IL-4 (R & D Systems, Minneapolis. MN) at final concentrations of 800 IU/mL and 1000 IU/mL, respectively. On day −2, recombinant human TNF-α (10 ng/mL), IL-6 (1000 IU/mL), and IL-1β (2 ng/mL) (R & D Systems) as well as PGE2 (1 μg/mL, StemCell Technologies) were added to cultured monocytes.

### (ii) CD8 Naïve T Cell Isolation

On day −1, autologous CD8 naïve T cells were isolated from PBMCs from HLA-A*02:01-expressing healthy donors using the EasySep™ Human Naïve CD8⁺ T Cell Isolation Kit II (StemCell Technologies) according to the manufacturer's instructions. Purity was assessed using fluorescently-labeled antibodies specific for CD8α (HIT8a, BioLegend), CD45RO (UCHL1, BioLegend), CD45RA (HI1100, BioLegend), CD56 (5.1H11, BioLegend), CD57 (HCD57, BioLegend), and CCR7 (G043H7, BioLegend); purity of naive CD8α⁺ T cells was >90%. Cells were rested overnight at 37° C., 5% CO₂ in T cell medium (X-VIVO 15 serum-free medium [Lonza, Rockland, MD] or LymphoONE [Takara, San Jose, CA] containing 10% human serum [Sigma Aldrich, St. Louis, MO], 1% penicillin-streptomycin [Thermo Fisher Scientific], 1% GlutaMAX [Thermo Fisher Scientific]), supplemented with 10 ng/ml recombinant human IL-7 (R & D Systems).

(iii) Co-Culture

On day 0, CD8 T cell purity was reassessed using the identical antibody panel as on day 3, and DC maturation was confirmed by upregulation of HLA-A2, CD80, CD83, and CD86 and downregulation of CD14. DCs were pulsed with 1 μM PRAME₄₂₅₋₄₃₃ peptide (SLLQHLIGL, GenScript [Piscataway, NJ]) as well as with additional peptides with various antigen specificities (i.e. multiplex screens) for 3 hours at 37° C., 5% CO₂. Pulsed DCs were co-cultured with rested CD8 naïve T cells in T cell medium supplemented with recombinant human IL-12 (10 ng/mL) and IL-21 (60 ng/mL) (R & D Systems). Co-cultures were supplemented with recombinant human IL-7 and IL-15 (R & D Systems) between days 3 and 10. Dextramer staining for PRAME₄₂₅₋₄₃₃-specific cells was performed on day 10 or 11, using an A*02:01 PRAME₄₂.a33 (SLLQHLIGL) dextramer (Immudex, Copenhagen, Denmark). CD8α and TCRα/β (IP26, BioLegend), and DAPI (Thermo Fisher Scientific) according to the manufacturer's instructions.

### (iv) Antigen-Specific Cell Sorting

On day 12, cells were collected and stained with A*02:01 PRAME₄₂₅₋₄₃₃ (SLLQHLIGL) dCODE Dextramer® (Immudex) together with dCODE Dextramers® (Immudex) specific for various other antigens according to the manufacturer's instructions. Cells were washed and then stained with antibodies specific for CD8α and TCα/β⁺, and with DAPI as on day 10-11, and dextramer-positive cells (CD8α⁺, DAPI⁻, TCRα/β⁺, dextramer⁺) were sorted using a Sony SH800S cell sorter (Sony Biotechnology, San Jose, CA). BigFoot Cell Sorter (Thermo Fisher Scientific), or MoFlo Astrios Cell Sorter (Beckman Coulter, Brea, CA). Sorted cells were subjected to single cell TCRα/β sequencing using the 10× Genomics platform (Pleasanton. CA).

### (v) Multiplexed NGS and Analysis Using the 10× Genomics Platform

Single-cell libraries were prepared according to the 10× Chromium Next GEM Single Cell 5′ Reagent Kit v2 (Dual Index) with feature barcode technology for cell surface protein & immune receptor mapping (10× Genomics, protocol CG000330 Rev A). Up to 15,000 cells were captured in droplets (GEMs) and processed according to manufacturer's instructions to obtain libraries that yielded VDJ and cell surface protein information. The fully assembled libraries were sequenced on an Illumina NextSeq2000 instrument (Illumina).

Sequenced VDJ and cell surface protein libraries were processed using the Cell Ranger 6.0.0 VDJ and COUNT pipelines (10× Genomics), respectively. The cell surface protein dataset measuring the dCODE Dextramers® (Immudex) was utilized to demultiplex the VDJ data to determine which epitope was recognized by each TCR. A target epitope was assigned to a cell barcode if for a given cell barcode, the total dextramer counts were greater than 10 and one dextramer accounted for >90% of the total counts. Utilizing the shared cell barcodes between the VDJ and cell surface protein libraries allows for identification of the target of each sequenced TCR.

c. Lentiviral Packaging and Quantification of Lentiviral Titer

Lenti-X GoStix Plus (Takara) was used to package and quantify PRAME₄₂₅₋₄₃₃ viral constructs. Briefly, PRAME₄₂₅₋₄₃₃ viral constructs were diluted 1:100 with PBS, 20 μl of PRAME₄₂₅₋₄₃₃ viral supernatant was added to a Lenti-X GoStix Plus cassette sample well, after which 80 μl of Chase buffer was added. A lateral flow test was run for 10 minutes, and a test band (T) appeared within 5 minutes, reaching maximum intensity at 10 minutes if sufficient lentivirus was contained with the sample. The control band (C) appeared when the test was functioning properly. After 10 minutes, proper alignment and focal length for imaging was achieved by using the outline of the cassette in the scanning window. Once proper alignment was achieved, the outline turns green, and the cassette was automatically scanned.

To calculate the actual IFU/ml for an unknown stock, a reference virus with known titer measured by CD8 expression was used (a virus stock for which the IFU/ml is known) and tested to obtain both an infectious unit value as well as a GoStix Value GV. The IFU/GV ratio for the reference virus was calculated, and the unknown sample using Lenti-X GoStix Plus was analyzed to obtain the GV (ng/ml p24) and to perform calculations [Formula: GV (unknown)×(IFU/ml)/GV (reference)=IFU/ml (unknown)] to determine IFU/ml.

d. Functional Evaluation of PRAME₄₂₅₋₄₃₃-Specific TCRs

### (i) Engineering T Cells to Express PRAME₄₂₅₋₄₃₃-Specific TCRs

Pan T cells were isolated from HLA-A*02:01-positive healthy donor PBMCs using the EasySep™ Human T Cell isolation kit (StemCell Technologies) as per the manufacturer's instructions. Isolated T cells were activated with ImmunoCult™ CD3/CD28/CD2 T cell activator cocktail (StemCell Technologies) and cultured overnight in complete T cell media (X-VIVO 15 [Lonza] or LymphoONE [Takara] supplemented with 5% human serum [Sigma Aldrich], 1% penicillin-streptomycin [Thermo Fisher Scientific], 1× GlutaMax supplement [Thermo Fisher Scientific], 5 ng/mL IL-7 [R & D Systems] and 50 IU/mL IL-2 [Sigma Aldrich]), 24 hours post-activation, T cells were transduced with PRAME₄₂₋₃₃ TCR viral supernatants at an MOI of 5. T cells were transferred either to G-Rex® plates (Wilson Wolf, St. Paul, MN), VECELL® 96-well plates (Cosmo Bio USA. Carlsbad, CA), or gas-permeable 96-well plates (Miltenyi Biotec) 24 hours post-transduction and expanded for a total of 6-10 days. T cell cultures were supplemented with fresh IL-2 (50 IU/mL) and IL-7 (5 ng/mL) every 2-3 days and/or split to maintain optimal cell densities.

### (ii) Flow Cytometry of Engineered PRAME₄₂₅₋₄₃₃ TCR-Transduced Pan T Cells

Engineered pan T cells were stained with HLA-A*02:01 PRAME₄₂₅₋₄₃₃ (SLLQHLIGL) (Immudex) dextramer, TCR α/β PE-Cy7 (IP26, BioLegend), CD8 PerCP-Cy5.5 (HIT8a, BioLegend), CD4 APC-Cy7 (OKT4, Biolegend), and CD34 Alexa Fluor 488 (QBEND/10, R & D Systems) and DAPI as per the manufacturer's instructions. Cells were then run on the CytoFLEX flow cytometer (Beckman Coulter) and analyzed using FlowJo software (version 10, TreeStar, Ashland. OR).

(iii) Cell Lines

The T lymphoblast cell line T2 (ATCC CRL-1992), amelanotic melanoma cell line Hs695T (ATCC HTB-137), epithelial malignant melanoma cell line A375 (ATCC CRL-1619), and lung adenocarcinoma cell line NCI-H1563 (ATCC CRL-5875) were purchased from the American Type Culture Collection (ATCC, Manassas, VA). The urothelial bladder carcinoma cell line 647V (ACC 414) was purchased from DSMZ (Braunschweig, Germany.) T2 cells were cultured in IMDM containing 20% heat-inactivated FBS and 1% penicillin-streptomycin (Thermo Fisher Scientific), Hs695T cells were cultured in EMEM containing 10% heat-inactivated FBS and 1% penicillin-streptomycin (Thermo Fisher Scientific), A375 cells were cultured in DMEM containing 10% heat-inactivated FBS and 1% penicillin-streptomycin (Thermo Fisher Scientific). NCI-H1563 cells were cultured in RPMI 1640 containing 10% heat-inactivated FBS and 1% penicillin-streptomycin (Thermo Fisher Scientific), and 647V cells were cultured in DMEM containing 15% heat-inactivated FBS, 1% penicillin-streptomycin, and 1× GlutaMax supplement (Thermo Fisher Scientific). Levels of PRAME and HLA-A for each cell line are shown in Table 5.

TABLE 5
PRAME-expressing cell lines used in functional assays

| | | PRAME | HLA-A |
|---|---|---|---|
| Cell Line | Cell line origin | TPM | TPM |
| Hs695T | Amelanotic melanoma | 242 | 521 |
| A375 | Epithelial malignant melanoma | 130 | 219 |
| NCI-H1563 | Lung adenocarcinoma | 9 | 3305 |
| 647V | Urothelial bladder carcinoma | 0.1 | 988 |

### (iv) Generation of Stable Cell Lines Expressing Incucyte® Nuclight Red

T2, Hs695T, A375, NCI-H1563, and 647V cells were transduced with Incucyte® NucLight Red Lentivirus Reagent (EF-1α promoter, puromycin selection) (Sartorius) in serum-free media at an MOI of 5. 24 hours post transduction, cells were washed and resuspended in their respective cell line media and cultured at 37° C., 5% CO₂. 3 days post-transduction, puromycin (Gibco, Waltham, MA) was added to the cultures at a pre-determined concentration (ranging from 0.5 μg/mL to 1 μg/mL) to select for transduced cells. Cultures were expanded under puromycin selection until they were at least 98% Nuclight Red-positive as determined by flow cytometric analysis.

### (v) In Vitro Cytotoxicity Assay

In vitro cytotoxicity assays for T2 cells were performed in 96-well flat-bottom tissue culture plates coated with poly-L-ornithine (Sigma Aldrich) for 30 minutes at RT, after which the coating solution was removed, and plates were allowed to dry for another 30 minutes at RT. In vitro cytotoxicity assays for adherent cell lines were performed in 96-well flat-bottom tissue culture plates without coating with poly-L-ornithine, in which the adherent cells were plated and allowed to attach overnight prior to addition of T cells. Where indicated, T cells were co-cultured with Nuclight Red-expressing Hs695T, A375, NCI-H1563, or 647V, or with Nuclight Red-expressing peptide-pulsed T2 cells (1 ng/ml of PRAME₄₂₅₋₄₃₃ peptide [SLLQHLIGL, Genscript]) at E:T ratios ranging from 10:1 to 1.25:1. Data were acquired on an Incucyte® S3 instrument (Sartorius. Bohemia, NY), and target cell growth was quantified on the Incucyte® S3 as a readout of T cell cytotoxicity. For determination of EC50 values, T2 cells were pulsed with a 10-fold serial dilution of PRAME₄₂₅₋₄₃₃ peptide from 1 μM to 10 fM, then co-cultured with T cells at a 5:1 ratio of T cells to targets. EC50 calculations were performed by fitting area-under-the-curve (AUC) data using Prism software (version 8.3.1. Prism, Irvine, CA).

### (vi) Cytokine Production Assay

T cells were co-cultured with Nuclight Red-expressing Hs695T, A375, NCI-H1563, or 647V, or with Nuclight Red-expressing peptide-pulsed T2 cells (1 ng/ml of PRAME₄₂₅₋₄₃₃ peptide [SLLQHLIGL, Genscript]) at an E:T of 1:1. Supernatants were harvested 24 hours later and frozen at −80° C. Supernatants were thawed and loaded on a multiAnalyte cartridge (ProteinSimple. San Jose, CA) to evaluate the levels of IFN-t. TNF-α, IL-2 and granzyme B using the Ella instrument (ProteinSimple).

e. Alloreactivity and Safety Screens

### (i) Generation of 96-Well-Based MHC-Expressing Arrays for Alloreactivity Screens

Endogenous HLA-A/B/C genes were knocked out in HEK293T cells using CRISPR-Cas engineering. Guide RNAs (gRNAs) were designed against sequences conserved across the HLA-A, HLA-B, and HLA-C loci using the multicrispr.net tool (Prykhozhij, 2015). The following guides were selected: CRISPR-ALL-1: CGGCTACTACAACCAGAGCG, CRISPR-ALL-2: AGATCACACTGACCTGGCAG, CRISPR-ALL-3: AGGTCAGTGTGATCTCCGCA, gRNAs were cloned into the LentiCRISPR V2 vector using BsmBI sites. HEK293T cells were transfected with plasmid guide constructs using Mirus TransIT (Mirus Bio, Madison, WI). After 7 days. MHC-knockout (MHC-KO) cells were sorted using a pan-MHC antibody (BioLegend). Single-cell clones were expanded, and the absence of MHC was verified by flow cytometry. B2M-knockout (B2M-KO) cells were used as a positive control for the complete absence of surface MHC expression. B2M was knocked out in HEK293T cells by electroporating CRISPR RNPs targeting B2M using the guide RNA: GGCCACGGAGCGAGACATCT.

MHC-null HEK293T cells were transduced with IncuCyte® NucLight™ Red virus (Sartorius). Transduced cells were sorted for NucLight™ Red expression using a Sony SH800S sorter (Sony Biotechnology). To generate an MHC-expressing array, MHC-null NucLight™ Red-expressing HEK293T cells were transduced with the most common 110 MHCs (pHAGE-EF1a-MHC-UBC-NAT) in individual wells in 96-well plates. Transduced cells were selected with nourseothricin (400 μg/ml) for one week. Cells expressing the most common 110 MHCs were passaged and stored in 96-well plates as an array. Expression of individual MHC alleles was verified by staining using a pan-MHC antibody (BioLegend).

To generate the positive control for the assay, MHC-null HEK293T cells were transduced with IncuCyte® NucLight™ Red virus (Sartorius) and sorted for NucLight™ Red expression using a Sony SH800 sorter. HLA-A*02:01 (pHAGE-EF1a-MHC-UBC-NAT) was then introduced into the cells using lentiviral transduction. Transduced cells were selected with nourseothricin (400 μg/ml) for one week. These cells were then transduced with a 90-mer construct (pHAGE-CMV-FHA-d1-EFS-AmCyan) expressing a fragment of PRAME which contains the PRAME₄₂₅₋₄₃₃ epitope (SLLQHLIGL). Transduced cells were then sorted for AmCyan expression using a Bigfoot Spectral Cell Sorter (Thermo Fisher Scientific).

### (ii) Lentiviral Packaging and Transduction

To package the lentiviruses of the 110 MHC expression constructs (pHAGE-EF1a-MHC-UBC-NAT), MHC-null HEK293T cells were plated at 75% confluency in 96 wells and transfected using jetPRIME transfection reagent (Polyplus, Illkirch, France). Individual MHC expression constructs were mixed with packaging plasmids (pREV/pTAT/pVSVG/pGAGPOL) and incubated with jetPRIME reagent according to the manufacturer's protocol, and DMEM medium was added at 24 hours post-transfection. Viral supernatants were harvested 48 hours after transfection and used for transduction of the 110 MHCs in a 96-well-based array format.

To package the lentiviruses of other constructs, Lenti-X cells (Takara Bio USA, Mountain View, CA) were plated at 75% confluency and transfected using jetPRIME transfection reagent (Polyplus). Expression constructs were mixed with packaging plasmids (pREV/pTAT/pVSVG/pGAGPOL) and incubated with jetPRIME reagent according to the manufacturer's protocol, and Opti-Pro SFM medium was added at 24 hours post-transfection. Viral supernatants were harvested 48 hours after transfection and were concentrated using either Vivaspin 20 centrifugal concentrators or Vivaflow 50 cassettes (Sartorius).

All viral transductions involving cell lines derived from HEK293T cells were performed in the presence of polybrene (4 μg/ml).

(iii) Generation of TCR 366-Expressing CD3 or CD8 T Cells for Alloreactivity and Safety Screens

Primary CD3+ or CD8+ T cells were isolated using the StraightFrom® Leukopak® CD3 Microbead Kit (Miltenyi Biotec) or StraightFrom® Leukopak@ CD8 Microbead Kit (Miltenyi Biotec), respectively, according to the manufacturer's protocol. Isolated cells were frozen in CryoStor® CS10 (Stem Cell Technologies) and stored in liquid nitrogen until use. On day −1, T cells were thawed, washed with complete T cell medium (RPMI 1640 supplemented with 10% heat-inactivated fetal bovine serum (FBS), 100 IU/mL penicillin, 100 μg/mL streptomycin, recombinant human IL-2 [50 U/mL, PeproTech. Cranbury, NJ], recombinant human IL-15 [5 ng/mL, R & D Systems], and recombinant human IL-7 [5 ng/mL, R & D Systems]. On day 0, T cells were washed and resuspended in fresh T cell medium and activated using ImmunoCult™ human CD3/CD28/CD2 T cell activator (5 uL/1×10⁶ CD8+ T cells, Stem Cell Technologies). On day 1, cells were washed and resuspended in fresh complete T cell medium and transduced with lentiviral particles to express TCR 366 at an MOI of 10. On day 2, cells were washed and resuspended in fresh complete T cell medium and expanded until day 5 in G-Rex®24 well plates (Wilson Wolf). On day 5, cells were harvested and resuspended in EasySep™ buffer (StemCell Technologies), and HLA-A*02:01 PRAME₄₂₅₋₄₃₃ dextramer-APC (Immudex) was added at a 1:50 dilution for 10 minutes on ice. Anti-CD34-Alexa Fluor®488 (Clone: QBEnd10, R & D Systems) was added at a 1:50 dilution and incubated for 20 minutes on ice. Cells were washed with EasySep™ buffer and resuspended in EasySep™ buffer containing DAPI at a 1:200 dilution. Live dextramer-binding cells (DAPI− Dextramer+ CD34⁺) cells were isolated by cell sorting (Beckman Coulter MoFlo Astrios EQ). Sorted cells were washed and resuspended in fresh complete T cell medium and expanded in G-Rex®10 flasks (Wilson Wolf) until day 12, at which point cells were frozen in CryoStor® CS10 and stored at liquid nitrogen until used.

Pan CD3+ T cells were restimulated (further expanded) in upright T25 flasks with 1×10⁶ T cells, 2×10⁷ irradiated (60 grays) allogeneic PBMCs, 50 U/mL recombinant human IL-2 (Peprotech), and 0.1 μg/mL anti-CD3 monoclonal antibody (OKT3, eBioscience, San Diego, CA). Half the volume of media was exchanged on days 2, 4, 5, and 6 with RPMI 1640 supplemented with 10% heat-inactivated fetal bovine serum (FBS), 100 IU/mL penicillin, 100 μg/mL streptomycin, and recombinant human IL-2 (50 U/mL, PeproTech, Cranbury, NJ). Cells were harvested for assay on day 7 for use in the alloreactivity screen.

CD8+ T cells expressing TCR 366 were thawed and restimulated by co-culturing T cells with irradiated (60 grays) allogeneic PBMCs in the presence of 0.1 μg/mL anti-CD3 (OKT3, eBioscience) and 50 U/mL recombinant IL-2 (Peprotech) in fresh T cell medium (RPMI 1640 supplemented with 10% heat-inactivated fetal bovine serum [FBS], 100 IU/mL penicillin, 100 μg/mL streptomycin, recombinant human IL-2 [50 U/mL, PeproTech]) in GRex®100 flasks (Wilson Wolf), 50 U/mL recombinant IL-2 was added to expanding cells every other day until day 6. On day 6, half of the culture medium was replaced with fresh T cell medium containing 50 U/mL recombinant IL-2. Cells were used for safety screens on day 7.

### (iv) Co-Culture for Alloreactivity Profiling

The assay was performed in triplicates. On day 5, target cells in the 96-well array were passaged and seeded in 384-well plates. On day 6, engineered CD3+ T cells expressing the recombinant TCR 366 or untransduced control T cells were added at an effector to target (E:T) ratio of 5:1 and incubated with target cells for 48 hours. Target cell numbers were measured over time using the IncuCyte® instrument by measuring the number of NucLight™ Red-positive cells. Cell inhibition at 48 h by the TCR 366 recombinant TCR on each MHC in the assay was calculated as 1-(Cell doubling[Incubated with TCR 366-expressing T cells]/Cell doubling[Incubated with untransduced control T cells]).

(v) QC of T Cell on-Target Killing

On day 5 of TCR 366 CD8 T cell restimulation, reporter cells (expressing the peptidome library) were labeled with Cell Trace Violet (Thermo Fisher) for 10 minutes at room temperature. The labeling reaction was quenched with a 5× excess of complete DMEM media (1×DMEM supplemented with 10% FBS, 100 IU/mL penicillin, 100 μg/mL streptomycin). After centrifugation, cells were seeded for subsequent assays. For the off-target screen, 4×10⁸ labeled reporter cells were seeded in a CellSTACK flask. For QC of T cell on-target killing, 25,000 reporter cells were seeded per well in a 96-well flat-bottom plate.

On day 6 of TCR 366 CD8 restimulation, T cells were tested for activity against PRAME₄₂₅₋₄₃₃ peptide. As a positive control, a fraction of reporter cells was pulsed with 100 ng/mL of PRAME₄₂₅₋₄₃₃ peptide (Genscript) for one hour. T cells were added to reporter cells at four effector:target (E:T) ratios (2:1, 1:1, 1:2, 1:4) in triplicate. After four hours of incubation at 37° C., cells were resuspended by pipetting up and down before data acquisition on a CytoFLEX flow cytometer (Beckman Coulter).

### (vi) Screen Co-Culture, Target Cell Enrichment and Sorting

On day 7 of TCR 366 CD8 expansion, T cells were added to library-transduced reporter cells and incubated at 37° C. for four hours. After incubation, all cells were harvested by trypsinization and centrifugation, cells were resuspended in 1× Annexin V binding buffer (Miltenyi Biotec) and centrifuged. Cells were resuspended with Annexin V magnetic microbeads (Miltenyi Biotec) in 1× Annexin V binding buffer (1 mL microbeads in 9 mL Annexin V binding buffer per 1×10⁹ total cells) and incubated at room temperature for 15 minutes. Cells were washed with 5× volume of Annexin V binding buffer and centrifuged. Cells were resuspended in Annexin V binding buffer and then divided over 2 “megareps” and filtered using a 70 μM cell strainer (Corning). Annexin V-labeled cells were positively selected using an AutoMACS Pro (Miltenyi Biotec). The elution of each “megarep” was further divided over four “replicates” for a total of 8 technical replicates per screen. IFP⁺ cells were sorted using a MoFlo Astrios EQ cell sorter (Beckman Coulter).

(vii) Next Generation Sequencing

Genomic DNA (gDNA) was extracted from sorted cells using the GeneJET Genomic DNA Purification Kit (Thermo Fisher Scientific). The antigen cassette was amplified from the extracted gDNA by PCR, then appended with sequencing adaptors and sample-specific index sequences in a second PCR reaction. Amplicons were sequenced on an Illumina NextSeq using the standard Illumina sequencing primer.

(viii) Data Analysis

Nucleotide sequences were mapped to individual nucleotide tiles. The proportion of read counts for each tile was calculated for each screen replicate (n=8) and for the input for each pool of transduced reporter cells, and enrichments of each tile were calculated by dividing the proportion of the tile in the screen replicate by the proportion of the tile in the input library. A modified geometric mean of the enrichment of an identical tile across the 8 screen replicates was used to identify reproducible screen hits. Specific MHC-binding epitopes for each tile above the threshold of 1.5-fold enrichment were predicted using NetMHC4.0. Candidate epitopes for each tile were selected by identifying predicted strong-binding epitopes shared across overlapping adjacent and redundant tiles that were enriched in the screen.

f. Safety Evaluation of TCR 366

Cancer cell lines were cultured in the media described in Table 6.

TABLE 6
Cancer cell lines and their culture media

| Cancer Cell | |
|---|---|
| Lines | Culture Medium |
| CaSki | RPMI-1640 + 10% HIFBS + 1X Pen/Strep |
| Loucy | RPMI-1640 + 10% HIFBS + 1X Pen/Strep |
| OVCAR-3 | RPMI-1640 + 10% HIFBS + bovine insulin |
| | (0.01 mg/mL) + 1X Pen/Strep |

### (i) Healthy Human Primary Cells Expressing Putative Off-Targets of TCR 366

Primary cells from healthy donors were thawed and cultured in the media described in Table 7 as per the manufacturer's instructions.

TABLE 7
Healthy human primary cells and their culture media

| Primary Cells | Cell lot # | Culture medium |
|---|---|---|
| Human Umbilical Vein Endothelial | 474Z035 | Endothelial Cell Growth Medium 2 |
| Cells (HUVEC) Growth Medium 2 | | (Ready-to-use) |
| Human Pulmonary Fibroblasts (HPF) | 474Z024.2 | Fibroblast Growth Medium 2 (Ready- |
| | | to-use) |
| Human Small Airway Epithelial Cells | 467Z033, | Airway Epithelial Cell Growth |
| (HSAEpC) | 467Z025.2 | Medium (Ready-to-use) |
| Human Bronchial Epithelial Cells | 469Z016 | Airway Epithelial Cell Growth |
| (HBEpC) | | Medium (Ready-to-use) |
| Normal human epidermal keratinocytes | 451Z014.1 | Keratinocyte Growth Medium 2 |
| (NHEK) | | (Ready-to-use) |
| Hepatocytes | HH1052, | UPCM—Universal Primary Cell |
| | HH1165 | Plating Medium |
| Human small intestinal epithelial cells | 201457 | Epithelial Cell Growth Medium |
| (HSIEpC) | | |
| Human bronchial smooth muscle cells | 21TL104462 | SmGM- 2 Smooth Muscle Cell |
| (HBSMC) | | Growth Medium -2 BulletKit |

### (ii) Cytokine Assay for Safety Evaluation of TCR 366

HLA-A*02:01⁺PRAME⁺ OVCAR-3 cells were used as a positive control, and HLA-A*02:01⁺PRAME⁻ CaSki or Loucy cells were used as a negative control, 24 hours prior to co-culture assays, OVCAR-3 and CaSki cells were detached from their culture flasks using TrypLE reagent, washed with media and seeded in 96-well flat-bottom culture plates at a density of 50,000 cells/well and allowed to adhere overnight. Loucy cells were washed on the day of co-culture and seeded in the 96-well flat-bottom culture plates at the same density. 24 hours prior to co-culture assays, HUVECs, HPFs, HSAEpCs, HBEpCs, HBSMCs, HSIEpCs and NHEKs were detached from their culture flasks using the DetachKit (PromoCell, Germany), washed and plated in their respective media at a density of 25,000 cells/well. Hepatocytes were thawed the day prior to co-culture as per the manufacturer's recommendation and plated at 56,000 cells/well and allowed to attach overnight. The following day, the primary target cells were pulsed with 100 ng/mL of PRAME₄₂₅₋₄₃₃ peptide for 2 hours or left unpulsed in their respective media. Following pulsing, wells were gently washed three times with target cell media. TCR 366 or NTD cells were then added at a density of 50,000 cells/well in complete T cell media without cytokines. Supernatants were collected 24 hours post-coculture and frozen at −80° C. Supernatants were thawed and analyzed for IFN-γ levels by loading on a Simple Plex Human IFN-gamma (3rd Gen) Cartridge (ProteinSimple, San Jose, CA) using the Ella instrument.

g. Materials

### Cell Lines

Lenti-X: Takara Bio USA, 632180
HEK293T cells: ATCC, CRL-3216
T2: ATCC, CRL-1992
Hs695T: ATCC, HTB-137
A375: ATCC, CRL-1619
NCI-H1563: ATCC, CRL-5875
647V: DSMZ, ACC 414
CaSki: ATCC, CRL-1550
OVCAR-3: ATCC, HTB-161
Loucy: ATCC. CRL-2629

### Healthy Human Primary Cells

Human Umbilical Vein Endothelial Cells (HUVEC) Growth Medium 2: PromoCell, A-12991 (Lot #474Z035)
Human Pulmonary Fibroblasts (HPF): PromoCell, C-12360 (Lot #474Z024.2)
Human Small Airway Epithelial Cells (HSAEpC): PromoCell, C-12642 (Lot #467Z033, 467Z025.2)
Human Bronchial Epithelial Cells (HBEpC): PromoCell, C-12640 (Lot #469Z016)
Normal Human Epidermal Keratinocytes (NHEK): PromoCell, C-12003 (Lot #451Z014.1)
Hepatocytes: In Vitro ADMET Laboratories, 82006 (Lot #HH1052, HH1165) Human Bronchial Smooth Muscle Cells (HBSMC): Lonza, CC-2576 (Lot #21TL104462)
Human Small Intestinal Epithelial Cells (HSIEpC): iXCells Biotechnologies, 10HU-237 (Lot #201457)

### Media and Supplements

X-VIVO 15, serum-free hematopoietic cell medium, with L-Glutamine, gentamycin and phenol red: Lonza, 04-418Q
LymphoONE™ T-cell expansion Xeno-Free medium, Takara, WK552
Human male AB serum (heat-inactivated): Sigma Aldrich, H3667-100ML
Penicillin streptomycin: Gibco, 15149-122
GlutaMAX supplement: Fisher Scientific, 35050061
RPMI-1640 medium: ATCC, 30-2001
Fetal bovine serum (FBS), heat-inactivated: Gibco, A3840102
IMDM: ATCC, 30-2005
RPMI medium 1640 (1×) [+]4.5 g/L D-glucose, [+]2.383 g/L HEPES buffer, [+] L-glutamine, [+]1.5 g/L sodium bicarbonate, [+]110 mg/L sodium pyruvate: Gibco, A10491-01
DMEM medium (1×) [+]4.5 g/L D-glucose, [+] L-glutamine, [+]3.7 g/L sodium bicarbonate: Thermo Fisher Scientific, 11965084
DMEM (1×) [+]4.5 g/L D-glucose, [+] L-glutamine, [−] Sodium Pyruvate: Gibco, 11965
OptiPRO SFM medium: Thermo Fisher Scientific, 12309019
EMEM medium: ATCC, 30-2003
Endothelial Cell Growth Medium 2 (Ready-to-use): PromoCell, C-22011
Fibroblast Growth Medium 2 (Ready-to-use): PromoCell, C-23020
Small Airway Epithelial Cell Growth Medium (Ready-to-use): PromoCell, C-21070
Airway Epithelial Cell Growth Medium (Ready-to-use): PromoCell. C-21060
Keratinocyte Growth Medium 2 (Ready-to-use): PromoCell, C-20011
SmGM-2 Smooth Muscle Cell Growth Medium −2 BulletKit: Lonza. CC-3182
Epithelial Cell Growth Medium: iXCells Biotechnologies, MD-0041
UCRM™—Universal Cryopreservation Recovery Medium, 50 mL: In Vitro ADMET Laboratories, 81015
UPCM™—Universal Primary Cell Plating Medium, 50 mL: In Vitro ADMET Laboratories, 81016
DetachKit: PromoCell, C-41220
TrypLE™ Express: Thermo Fisher Scientific, 12605-010

### Buffers

EasySep™ buffer: StemCell Technologies, 20144
DPBS (no Ca2+/Mg2+): Gibco, 14190-122

### Cytokines

Recombinant human IL-2: Sigma Aldrich, 11147528001
Recombinant human IL-2: PeproTech, 200-02
Recombinant human IL-7: R & D Systems, 207-IL-025
Recombinant human IL-15: R & D Systems, 247-ILB-005

### Kits

EasySep™ Human T Cell Isolation Kit: StemCell Technologies: 17951
Ella simple plex kit for 32 samples: ProteinSimple, SPCKB-PS-003027
StraightFrom™ Leukopak® CD8 Microbead Kit: Miltenyi Biotec, 130-117-019
StraightFrom® Leukopak® CD3 Microbead Kit: Miltenyi Biotec, 130-122-365
Annexin V MicroBead Kit: Miltenyi Biotec, 130-090-201

### Antibodies and Staining Reagents

CD3 monoclonal antibody (OKT3), functional grade: eBioscience 16-0037-81
Anti-human CD8a PerCp-Cy5.5 (clone: HIT8a): BioLegend: 300924
Anti-human CD4 APC-Cy7 (Clone: OKT4): BioLegend: 317418
Anti-human CD56 PE (clone: 5.1H11): BioLegend: 362508
Anti-human CD57 PE (clone: HCD57): BioLegend: 322312
Anti-human CD34 Alexa Fluor 488: (Clone: QBEND/10): R & D Systems. FAB7227G
Anti-human HLA-A2 PE (clone: BB7.2): BioLegend, 343306
Anti-human TCRα/β PE-Cy7 (clone: IP26): BioLegend: 306720
Anti-human HLA-A,B,C APC Antibody: BioLegend, 311410
Fixable viability Dye eFluor 660 (APC Channel) Thermo Fisher Scientific: 65-0864-14
Cell Trace Violet (CTV): Thermofisher, C34557
DAPI: Thermo Scientific: Thermo Scientific, 62248
Cytofix: BD Biosciences, 554655

### Peptides

PRAME₄₂₅₋₄₃₃ (SLLQHLIGL): Genscript

### Dextramers

HLA-A*02:01 PRAME₄₂₅₋₄₃₃ (SLLQHLIGL) dextramer: Immudex, WB4074
HLA-A*02:01 PRAME₄₂₅₋₄₃₃ (SLLQHLIGL) dCODE® dextramer: Immudex, WB4074-PfBC0409

### CRISPR-Cas9 and Electroporation Reagents

Alt-R® S.p. Cas9 Nuclease V3 (Lot #0000417827): IDT, 1081059
Alt-R® CRISPR-Cas9 tracrRNA (Lot #0000415438): IDT, 1072534
4D-Nucleofector™ Core Unit: Lonza, AAF-1002B
4D-Nucleofector™ X Unit: Lonza, AAF-1002X
SE Cell Line 4D-Nucleofector™ X Kit L: Lonza V4XC-1012

### Tissue Culture Plates

G-Rex®24-well plate: Wilson Wolf, 80192M
G-Rex® 6-well plate: Wilson Wolf, 80240M
G-Rex®100: WilsonWolf, 800400
VECELL® 96-well plate: Cosmo Bio Co., VCL-V96WGPB-10-EX
Gas-permeable culture plates (96-well), Miltenyi Biotec: 150-000-364
TC-treated 6-well plate: Corning Costar, 3506
TC-treated 24-well plates: Corning Costar, 3524
96-well flat-bottom plates: Corning Costar, 3595
384-well flat-bottom plates: Corning Costar, 3764
CellAdhere™ Collagen I-Coated, 96-well flat-bottom plate: StemCell Technologies, 100-0366
CellSTACK®, 5 Chamber with Vent Caps, Corning, 3313
70 μM Nylon Cell Strainer, Corning, 431751

### Packaging and Lentivirus Production Reagents

Lenti-X GoStix Plus: Takara, 631281
jetPRIME transfection reagent: Polyplus-transfection, 114-75
Vivaspin 20: Sartorius. VS2041
Vivaflow 50 cassettes: Sartorius, VF05P4

### Leukopak Processing Reagents:

Multi-24 Column Block: Miltenyi, 130-095-692
Lymphocyte Separation Medium: Corning, 25-072-CV

### Other Reagents

ImmunoCult™ Human CD3/CD28/CD2 T cell activator: StemCell Technologies, 10970
Poly-L-ornithine solution 0.01%: Millipore-Sigma. P4957-50ML
CryoStor® CS10: StemCell Technologies, 07930
ViaStain AO/PI staining solution in PBS: Nexcelom Bioscience. CS2-0106-5 mL
Nourseothricin: GoldBio, N-500-1
Puromycin: Gibco, A11138-03
Mirus TransIT, Mirus Bio MIR 2704
Polybrene: EMD Millipore, TR-1003-G
Incucyte® NucLight Red Lentivirus Reagent (EF-1 Alpha Promoter, Puromycin selection), Sartorius, 4476
ViaStain AO/PI staining solution in PBS: Nexcelom Bioscience. CS2-0106-5 mL

### Example 2: Discovery of TCRs Specific for PRAME₄₂₅₋₄₃₃ (SLLQHLIGL)

TCRs specific for 5 different A*02:01-restricted PRAME-derived epitopes were discovered using the proprietary ReceptorScan platform. Using an activation-based screening technology termed ActivScan, the most functional TCRs from a library of 1.300 PRAME-specific TCRs were identified to select for TCRs with greatest avidity and expression. These highly active TCRs were examined for their cytotoxic function using a panel of PRAME-expressing A*02:01-positive cell lines. Lead TCRs were assessed for potential off-target reactivity using the proprietary genome wide screen platform, in which off-target recognition of antigens derived from all proteins that comprise the human proteome was evaluated. Safety was further confirmed by examining alloreactivity to high-frequency class I HLAs and by testing TCR reactivity to a panel of normal primary human cells. Lastly, TCR-T cells are tested for their ability to control tumor growth in vivo using PRAME-expressing xenograft mouse models.

871 million naïve CD8⁺ T cells were screened from 16 unique healthy donors in ReceptorScan to identify 5706 TCRs specific for 5 PRAME epitopes. PRAME₄₂₅₋₄₃₄-specific TCRs demonstrated superior recognition of a PRAME-expressing cell line compared to all other PRAME epitopes tested. Following selection of high-expressing and high avidity PRAME₄₂₅₋₄₃₄-specific TCRs in ActivScan, TCRs were evaluated for their cytotoxic function, and two TCRs compared favorably to a clinical-stage benchmark TCR with respect to cytotoxicity, cytokine release, and T cell proliferation. Safety assessment demonstrated that few off-target peptides were recognized by lead TCRs, minimal alloreactivity was observed to 110 allotypes tested, and no reactivity to normal primary human cells was found. PRAME₄₂₅₋₄₃₄-specific TCR-T cells are believed to be able to control tumor growth in vivo following infusion into immunodeficient mice implanted with PRAME-expressing xenografts.

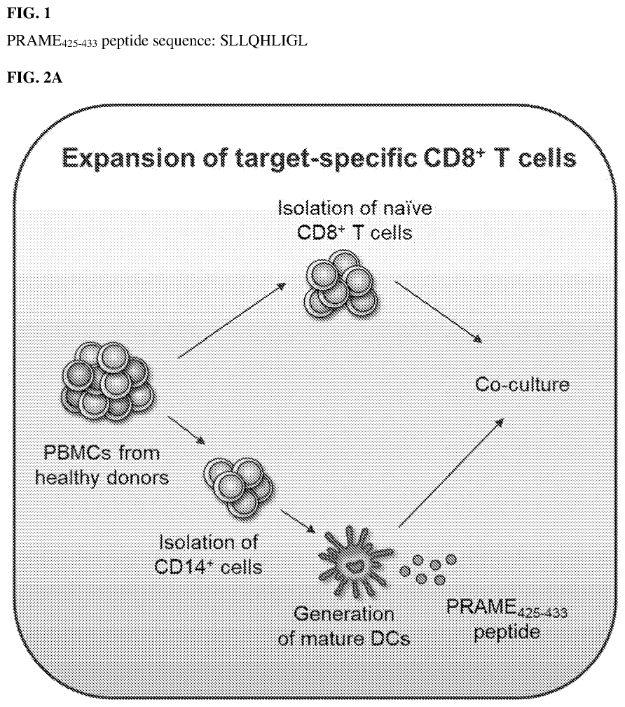

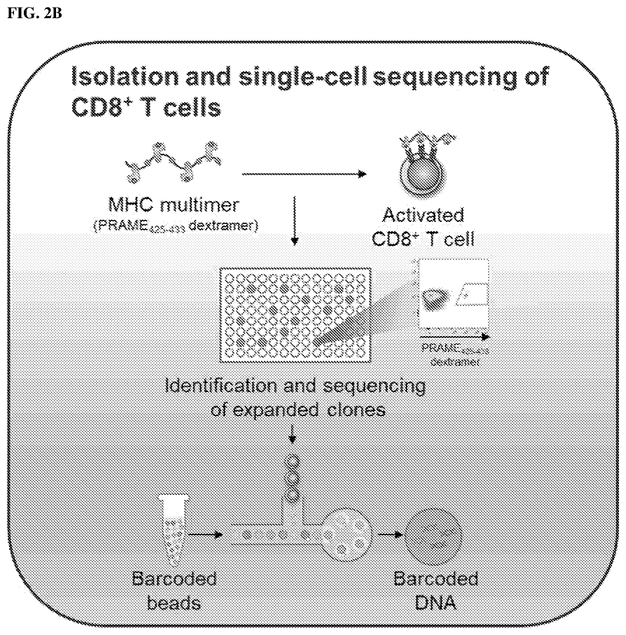

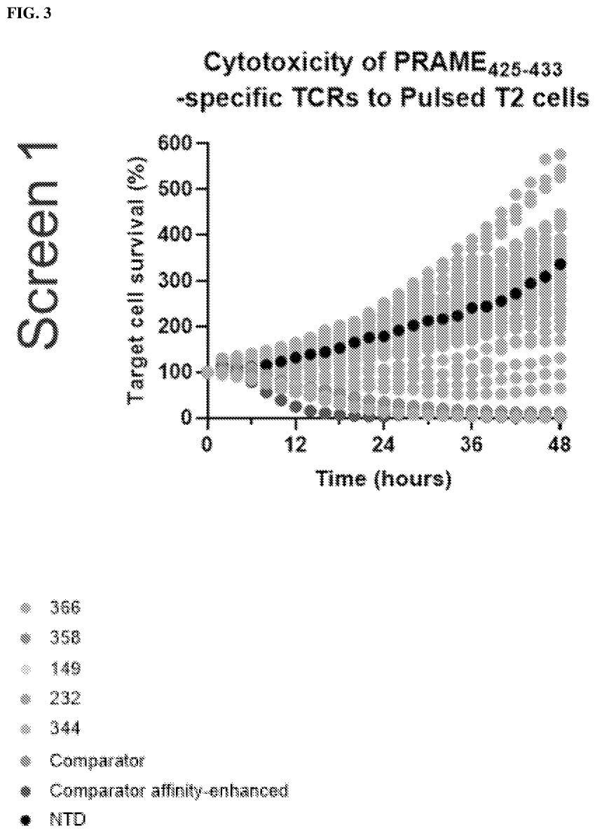

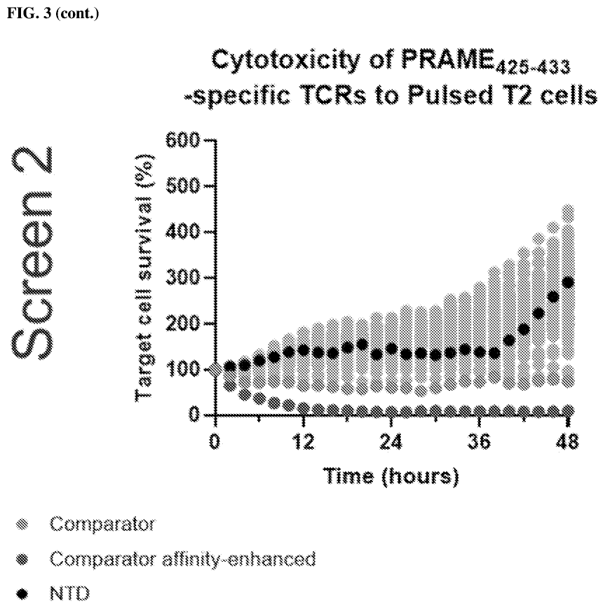

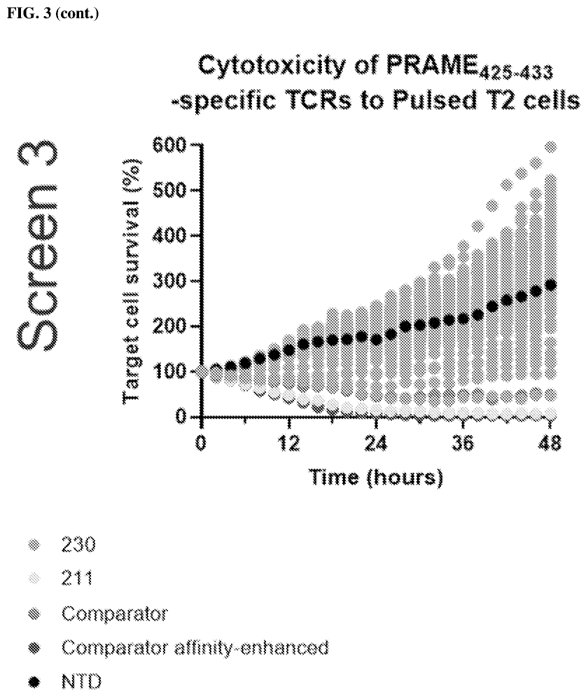

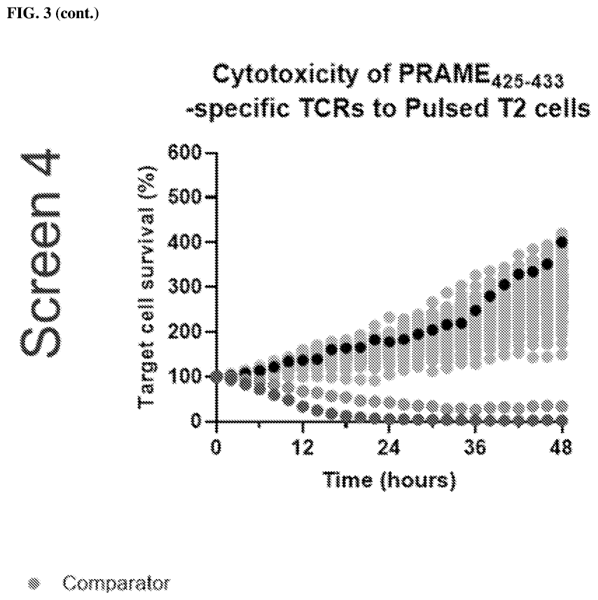

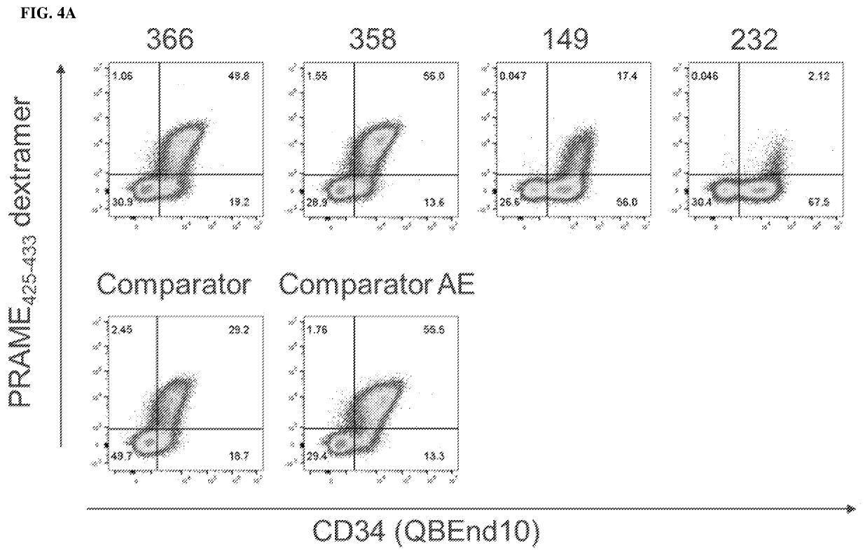

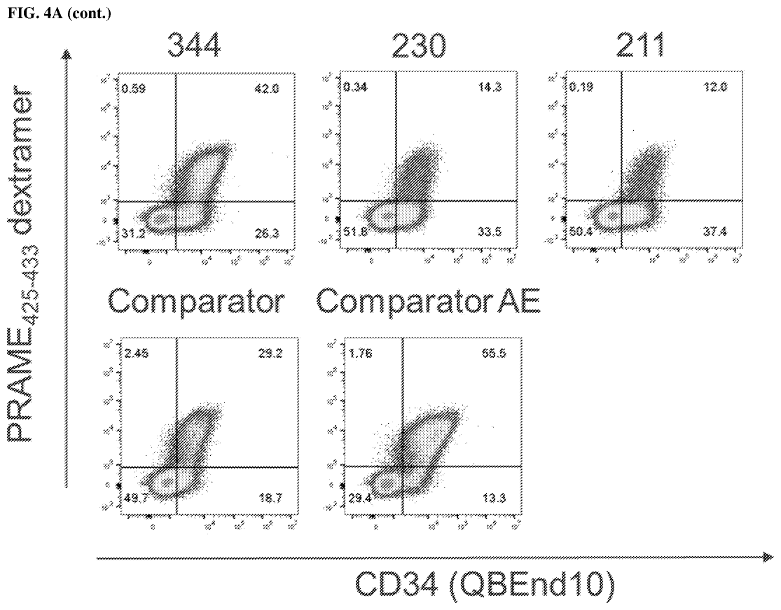

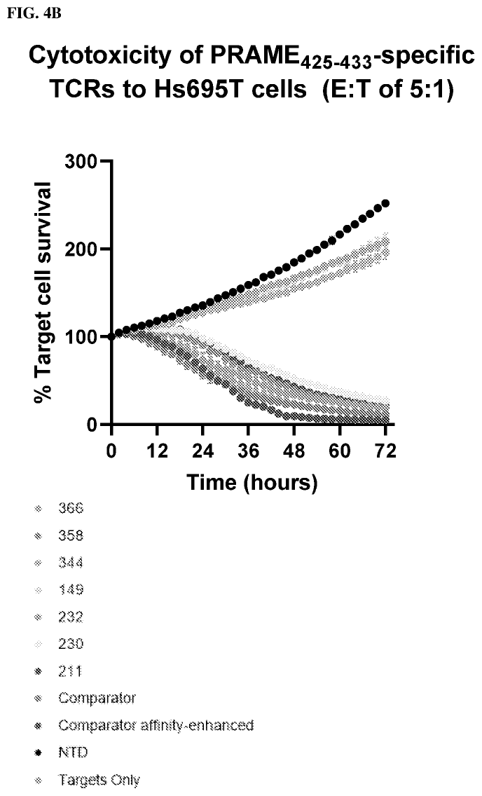

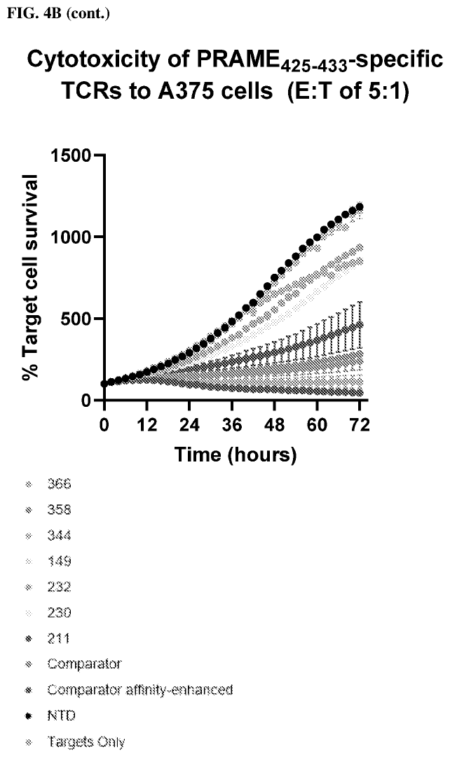

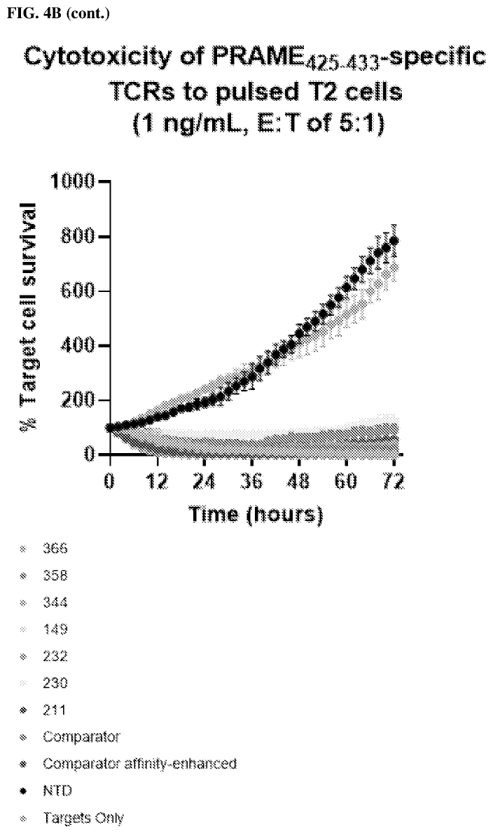

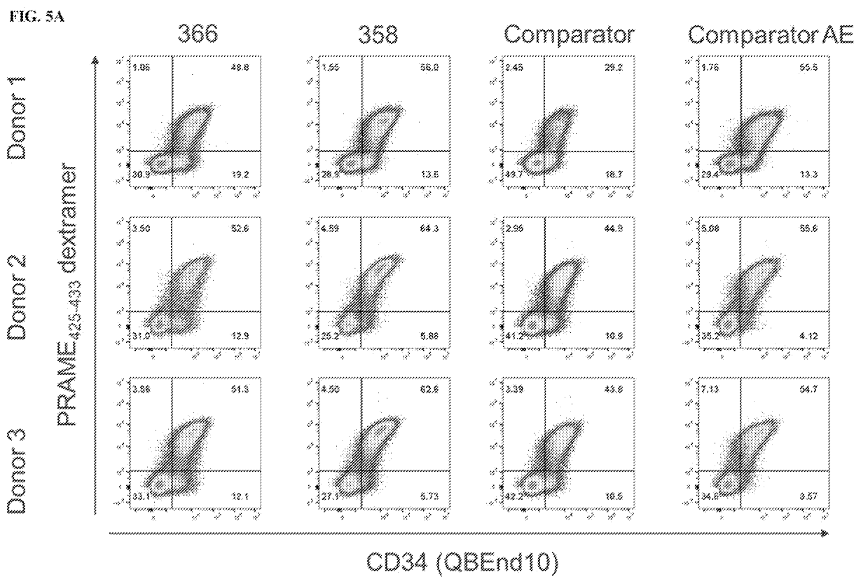

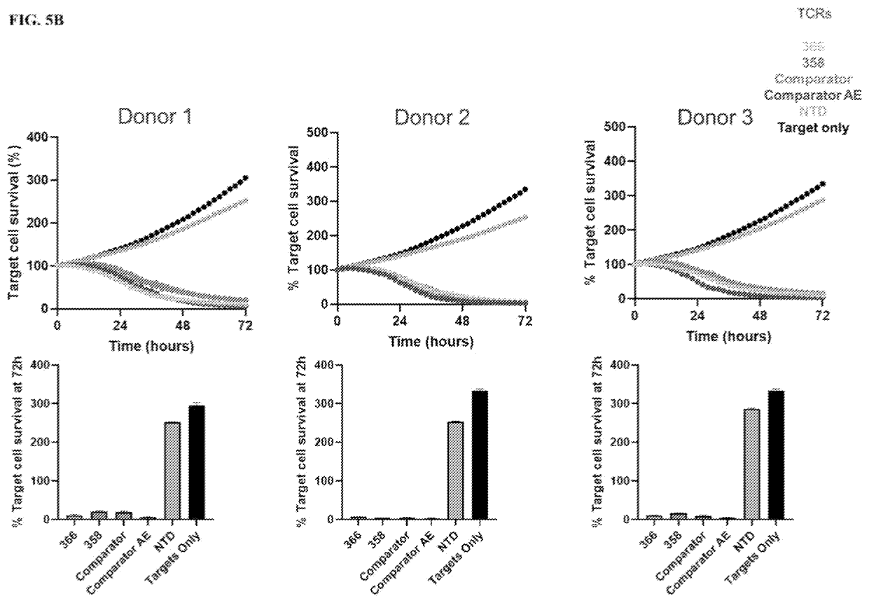

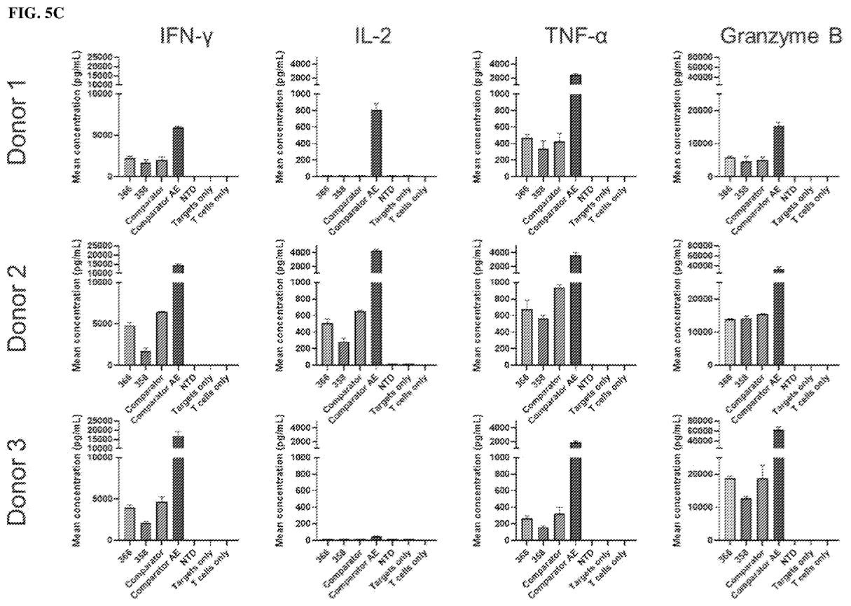

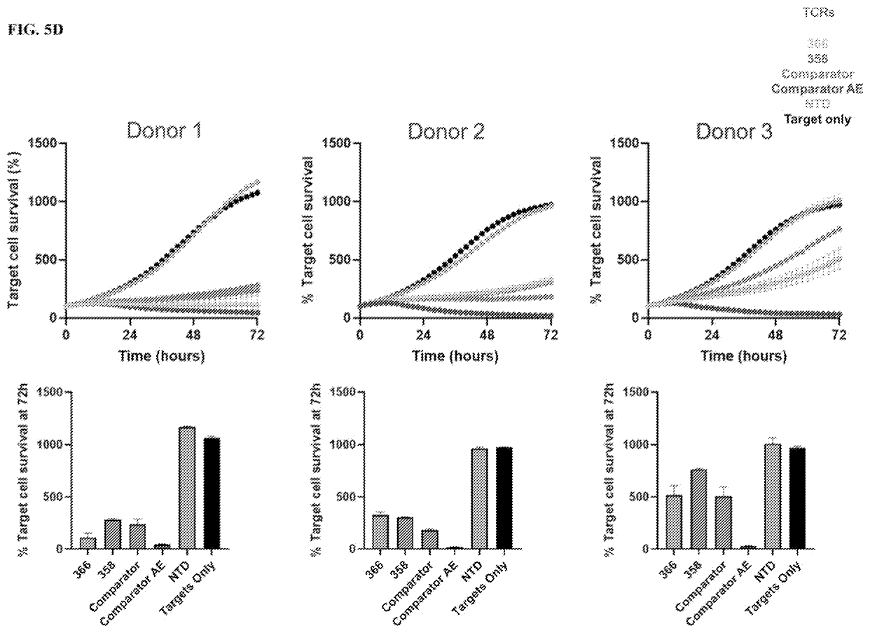

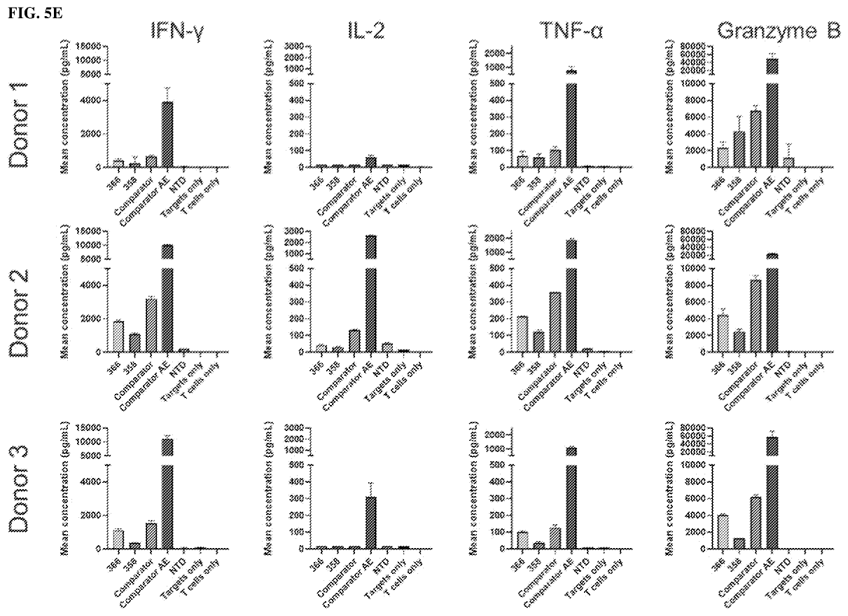

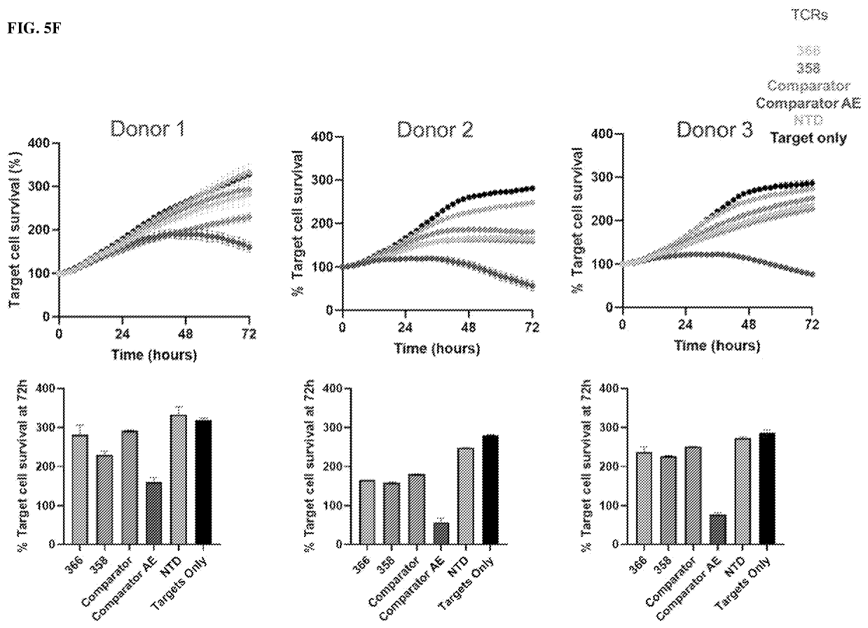

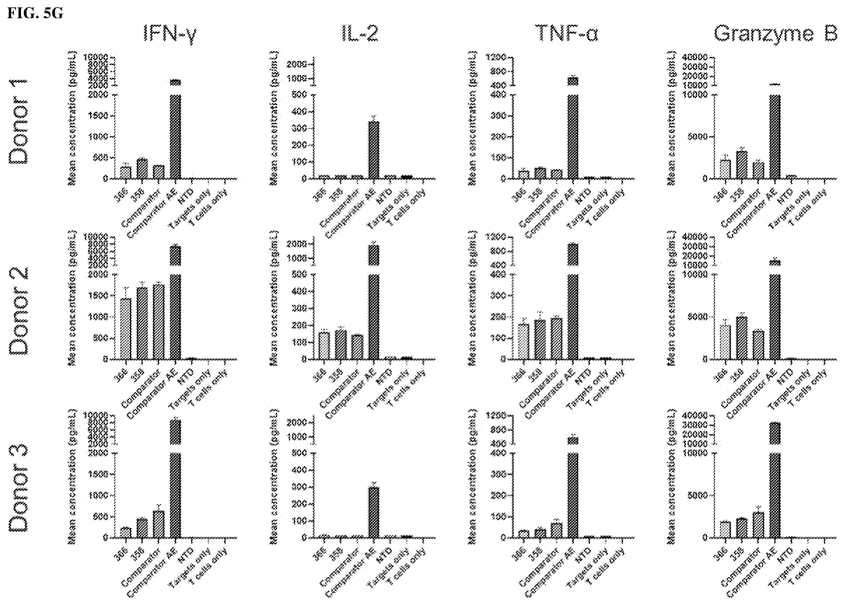

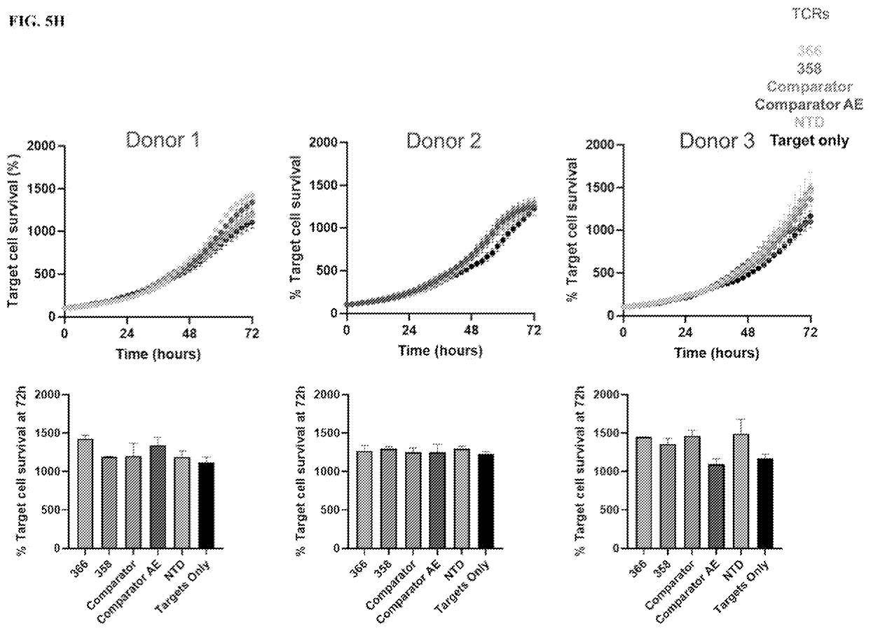

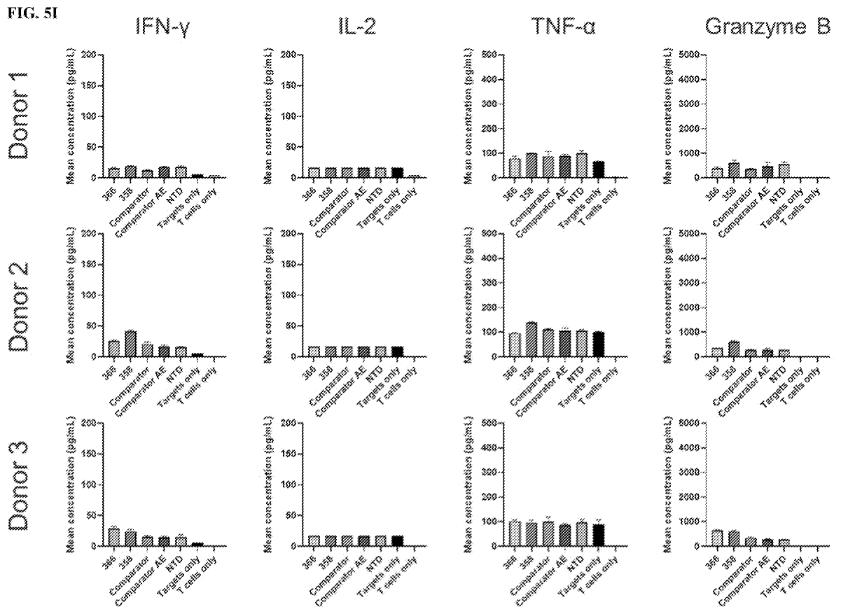

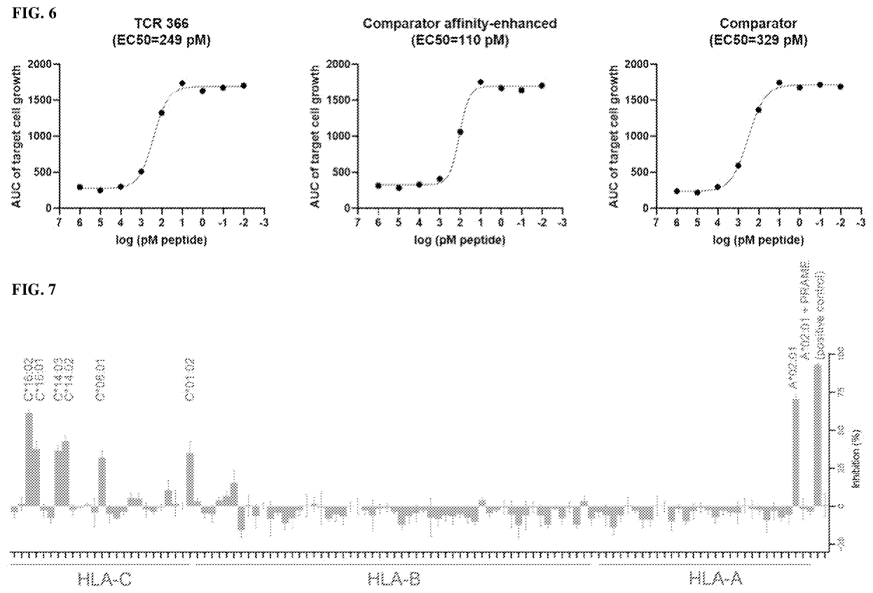

PRAME₄₂₅₋₄₃₃-specific TCRs were discovered using the HLA-A*02:01-restricted epitope SLLQHLIGL (FIG. 1), 392 PRAME₄₂₅₋₄₃₃ (SLLQHLIGL)-specific TCRs were identified using the proprietary ReceptorScan platform (FIG. 2A and FIG. 2B). Screening of 392 PRAME₄₂₅₋₄₃₃ TCRs identifies 7 TCRs with cytotoxic activity favorable to comparator TCR (FIG. 3). These 7 out of 392 TCRs were selected for further evaluation for surface expression and cytotoxic potential against PRAME-expressing cell lines. Three TCRs were shown to bind PRAME₄₂₅₋₄₃₃ (SLLQHLIGL) dextramer and were evaluated further in an in vitro cytotoxicity assay, in which they were compared to comparator TCRs (comparator AE: comparator affinity-enhanced) (FIG. 4A). TCRs 366 and 358 showed favorable activity to the comparator TCR, particularly in control of A375 cell growth, in which PRAME expression is lower (FIG. 4B). FIG. 5A-FIG. 5I provide summary results demonstrating that TCR 366 and 358 shows favorable surface expression, cytotoxicity, and cytokine production relative to the comparator TCR. The EC50 of TCR 366 is also favorable relative to the comparator TCR (FIG. 6).

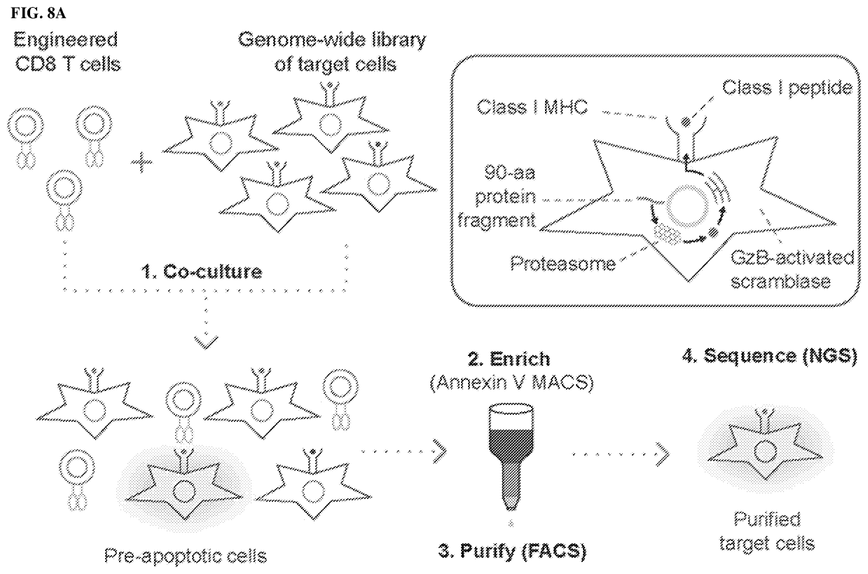

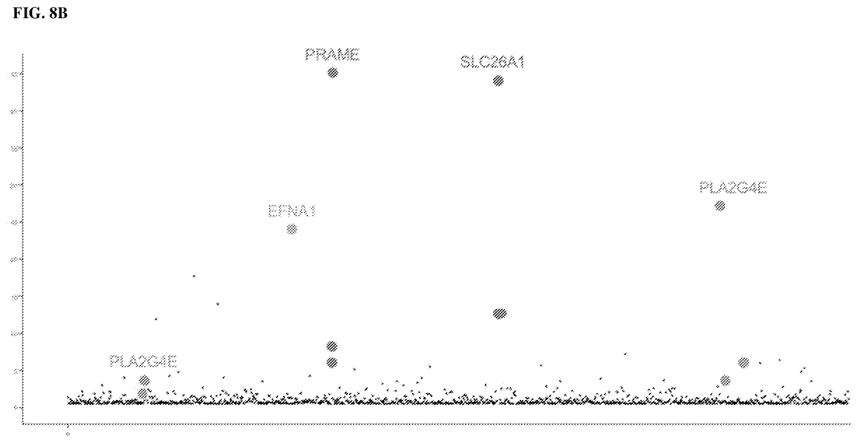

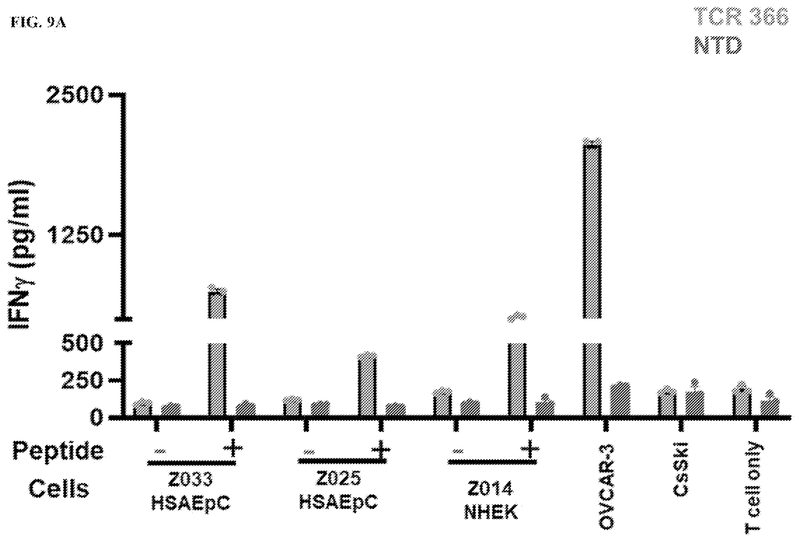

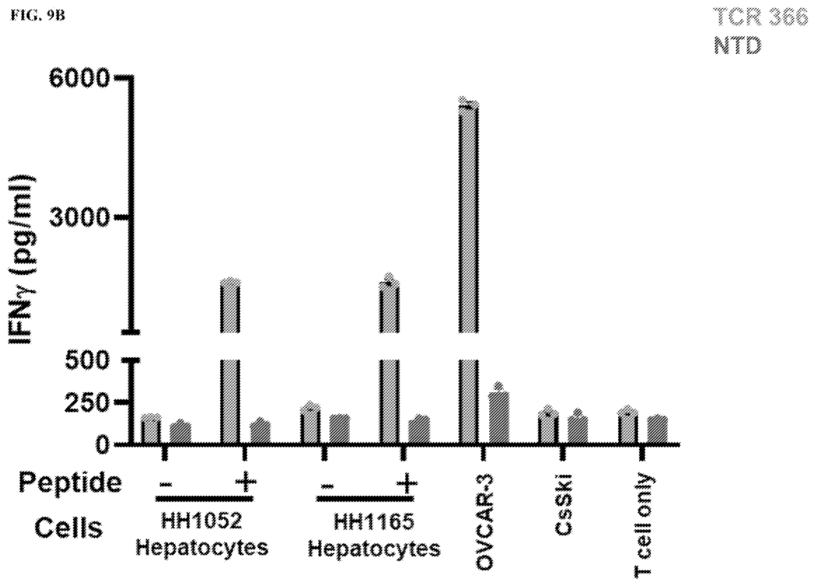

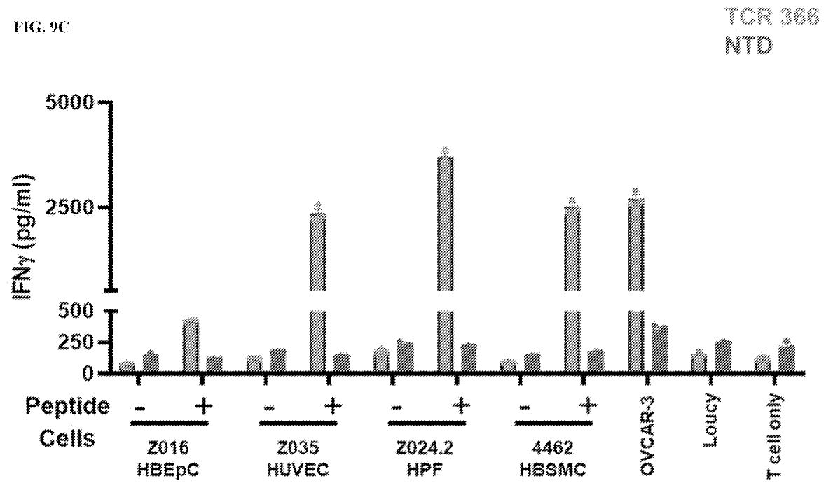

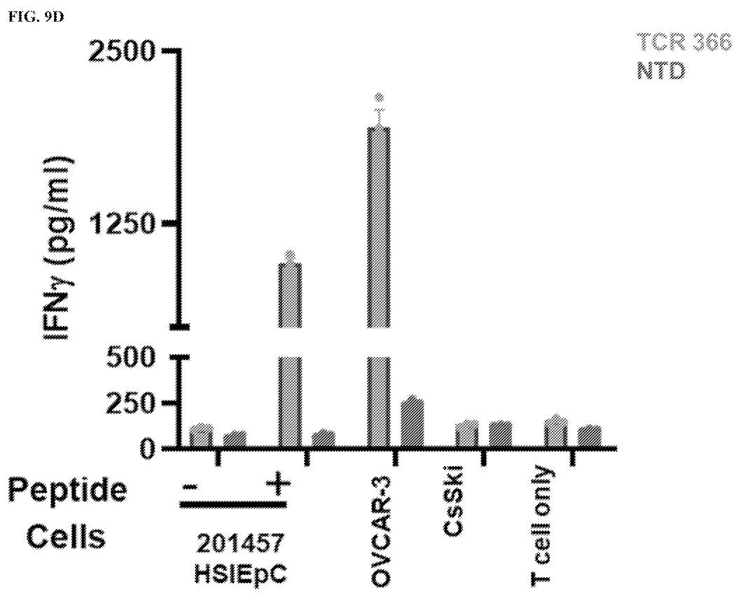

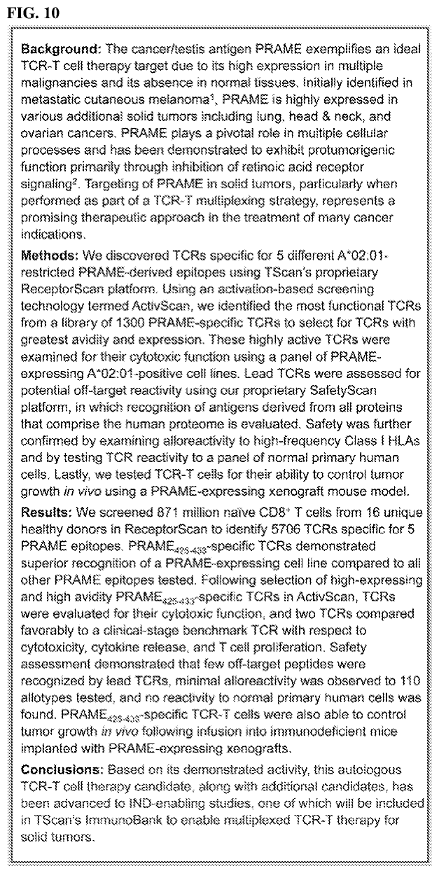

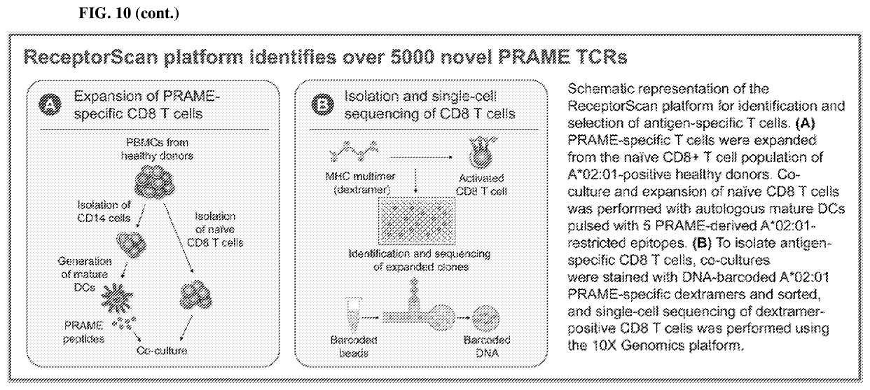

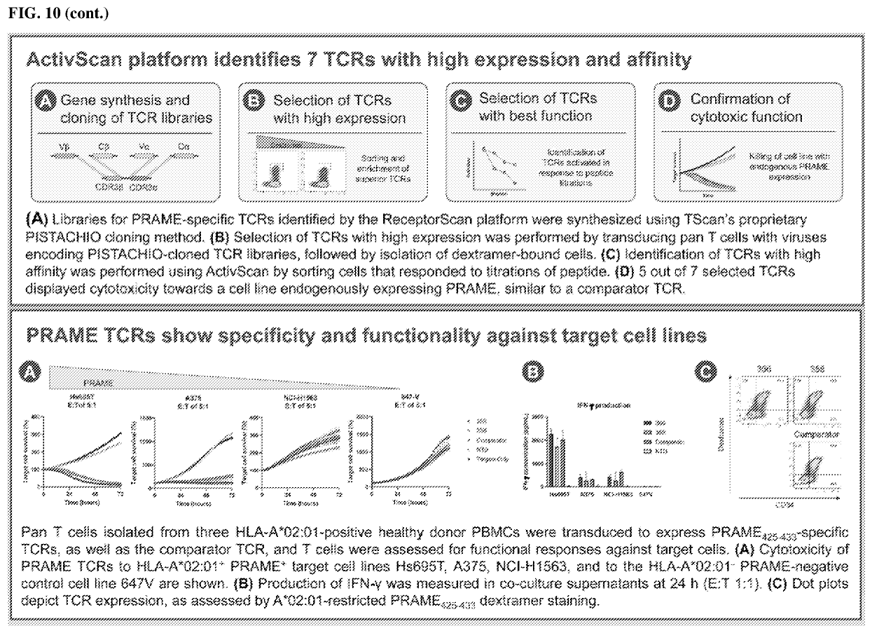

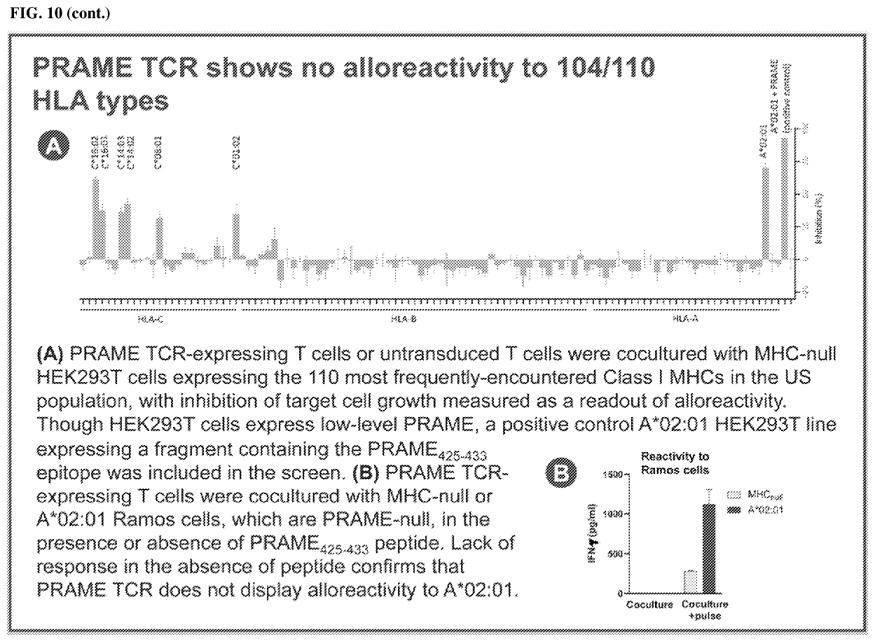

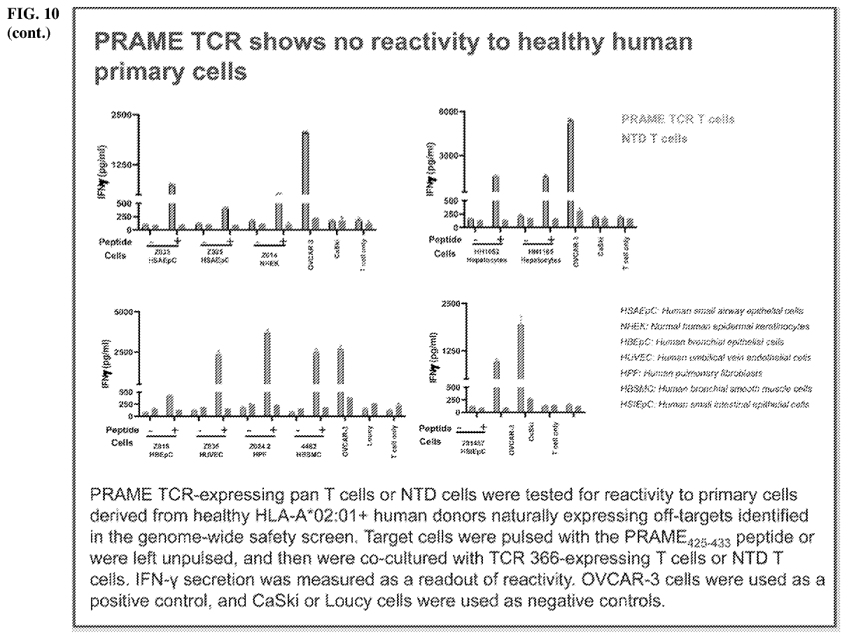

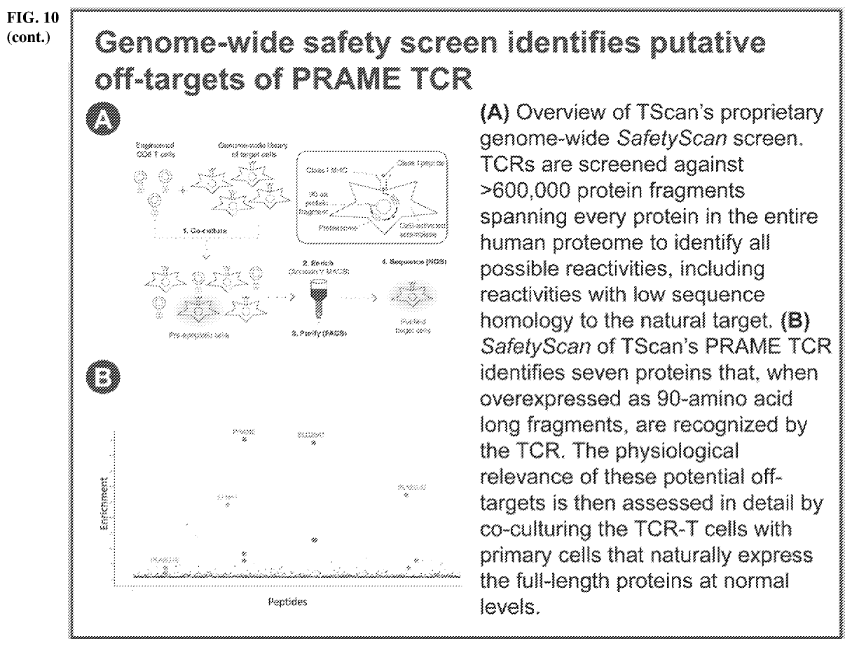

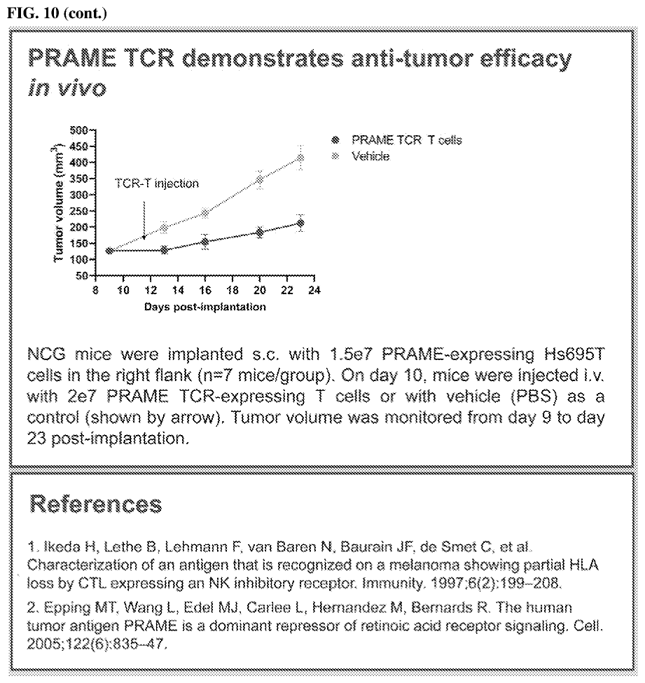

In addition, PRAME₄₂₅₋₄₃₃-specific TCR 366 was screened in an alloreactivity assay. TCR366 exhibited minimal allo-reactivity generally as defined by target cell inhibition of more than 20% (e.g., no detectable allo-reactivity to 103 of 110 different HLA types tested) (A*02:01, C*16:02, C*16:01. C*14:03, C*14:02, C*08:01, and C*01:02 at low levels) (FIG. 7). The proprietary genome-wide screen data of TCR 366 identified putative off-targets in a screen of >600,000 protein fragments spanning every wildtype (w.t.) human protein (FIGS. 8A and 8B). TCR 366 showed no reactivity to healthy human primary cells (FIG. 9). Thus, a variety of PRAME₄₂₅₋₄₃₃-specific TCRs having desirable characteristics (e.g., target recognition, cell surface expression, cytotoxic function, low alloreactivity, etc.) have been identified and described herein. Based on its demonstrated activity in vitro and expected activity in vivo, this autologous TCR-T cell therapy candidate. TCR366, also known as TSC-203-A02, has been advanced to IND-enabling studies. Data and results are further summarized in FIG. 10.

### Example 3: Further Confirmatory Characterization of TSC-203-A0201 Action

The following representative Examples 3-8 further confirm the data and results presented in working examples 1-2 and continue to be based, in part, on the recognition that adoptive cell transfer with genetically engineered T cells holds great promise for treating solid tumors. Patients positive for particular HLA alleles of interest, such as HLA-A*02:01, are amenable to treatment with TCRs recognizing epitopes of a given target presented by such HLAs, such TSC-203-A0201.

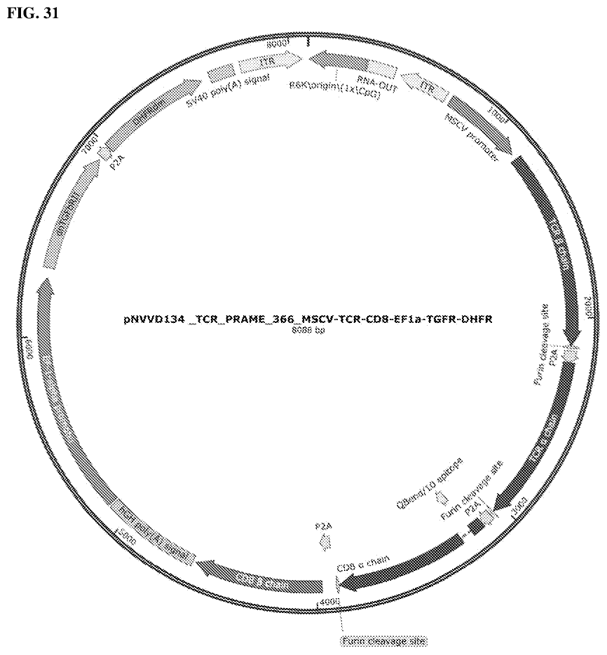

For representative Examples 3-8, process-representative TSC-203-A0201 TCR-T cells (e.g., helper (CD4⁺, now CD8⁺/CD4⁺) and cytotoxic (CD8⁺) T cells) were engineered by transposon/transposase-mediated gene delivery of vector pNVVD134 (i.e., pNVVD134_TSC-203-A02_TCR-366_MSCV-TCR-366-CD8-EF1α-dnTGFbRII-DHFR as shown in FIG. 31) (unless otherwise indicated), to express (1) a recombinant TCR (e.g., the recombinant TCR specific to the PRAME-derived peptide SLLQHLIGL presented on HLA-A*02:01), (2) recombinant CD8α and CD8β co-receptors to maximize the efficacy of the therapeutic product, (3) a CD34-derived epitope tag fused on the N-terminus of CD8a to facilitate tracking of engineered cells in vitro and in vivo, (4) a mutated form of dihydrofolate reductase (DHFRdm) protein to facilitate enrichment of engineered cells during the manufacturing process, and (5) a dominant negative type II TGFβ receptor (DN-TGFγRII) to further address tumor microenvironment-mediated immune suppression. Such cells can be manufactured using known techniques and, for the present example, were generated through isolation of peripheral blood mononuclear cells (PBMC) from a fresh apheresis product, delivery of transposase mRNA and vector transposon npDNA by electroporation. T cell activation and culture, engineered cell enrichment via addition of methotrexate (MTX) in culture medium (selective growth advantage of engineered cells conferred by DHFRdm expression from transposon vector), cell washing to remove MTX, culture expansion, and culture wash, formulation, cryopreservation.

TSC-203-A0201 TCR-T cells were analyzed by flow cytometry to characterize the cell composition. The clinically representative TSC-203-A0201 TCR-T cells contain engineered (i.e., CD34⁺) helper (CD4⁺, now CD4⁺/CD8⁺) and cytotoxic (CD8⁺) T cells. The engineered helper and cytotoxic T cells express the recombinant TCR able to recognize the PRAME-derived peptide SLLQHLIGL bound to HLA-A*02:01 as demonstrated by the binding of the relevant dextramer. Engineered TCR-T cells also express DN-TGFβRII.

Functional data were generated to characterize the mechanism of action of TSC-203-A0201. The data demonstrate that clinically representative TSC-203-A0201 TCR-T cells from at least 3 independent batches (subjects) react to the PRAME-derived peptide SLLQHLIGL in a dose-dependent manner when presented by target cells on the HLA-A*02:01 MHC in peptide pulse experiments. Three representative, independent batches of TCR-T cells generated from different donors were tested for their reactivity against their cognate peptide/MHC. As described further below, TSC-203-A0201 TCR-T cells were co-cultured with peptide pulsed T2 cells, a cell line unable to present endogenous peptide on class I MHC molecules due to a deficiency in the peptide transporter TAP (Steinle & Schendel (1994) Tissue Antigens 44:268-270). T2 cells were pulsed with a titration of the PRAME-derived peptide SLLQHLIGL and TCR reactivity was determined by the peptide dose-dependent cytotoxicity against the T2 cells. This study confirmed that TSC-203-A0201 specifically reacts to cells presenting the PRAME-derived peptide SLLQHLIGL presented on HLA-A*02:01.

When encountering a target cell naturally expressing PRAME and HLA-A*02:01, TSC-203-A0201 TCR-T cells engage in the secretion of inflammatory cytokines and of granzyme B and mount a proliferative response detected both in the engineered cytotoxic and helper T cell subpopulations. The TCR-T cells eventually kill these target cells naturally presenting the PRAME-derived peptide on HLA-A*02:01. Here, a panel of target cancer cell lines naturally expressing PRAME and HLA-A*02:01 was used in co-culture assays with the 3 independent batches of TSC-203-A0201 TCR-T cells to evaluate the biological outcomes regarding TSC-203-A0201 TCR engagement. Untransfected (UTF) control T cells obtained from matching donors were used as negative controls. Untransfected (UTF) control T cells were also produced from the isolated PBMCs used to generate each batch of TSC-203-A0201 process-representative material tested. The UTF cells did not undergo electroporation or MTX based enrichment, but were similarly activated and cultured as the TSC-203-A0201 TCR-T cells. Target cells positive for HLA-A*02:01 expression but negative for PRAME were used as additional negative controls. Multiple readouts were used to evaluate the functional engagement of the TCR-T cells, including: (1) secretion of inflammatory cytokines (IFN-γ, TNF-α and IL-2) and granzyme B in the co-culture supernatant was evaluated 24 hours post co-culture; (2) proliferation of the engineered T cells (both CD4⁺ and CD4⁻) was assessed after −4 days post co-culture; and (3) selective cytotoxic activity was examined over 3 days of co-culture.

In addition, the target-dependent function of TSC-203-A0201 is insensitive to physiological levels of TGFβ (e.g., active even in the presence of TGFβ, an immunosuppressive cytokine typically observed in the microenvironment of solid tumors), a function provided by the expression of the DN-TGFβRII.

Materials and methods for preparing effector T cells and target cells, as well as setting up co-cultures, are described herein. T cells were thawed in a 37° C. water bath and washed once with cytokine-free T cell medium to remove cryopreservation reagents prior to being resuspended in complete T cell medium. T cells were then seeded in a G-REX® 6-well plate at a density of 1-2 E6 live cells/mL and allowed to recover in a humidified incubator at 37° C. and 5% CO₂ for 16-24 hours prior to co-culturing. On the day of co-culture, T cells were harvested, washed, and resuspended in cytokine-free T cell medium at an assay-dependent cell density. Target cells were prepared similarly.

Similarly, target cells (Table 8) were prepared. For example, cancer cell lines were thawed in a 37° C. water bath and washed once with their respective cell culture medium to remove cryopreservation reagents. Cells were subsequently resuspended in cell culture medium and cultured following standard procedures in 75 cm² flasks (adherent target cells) or G-REX® wells (suspension cells) in a humidified incubator at 37° C., and 5% CO₂. The cancer cell lines were maintained in culture at least one passage, and no longer than 3-4 weeks prior to the initiation of the co-culture with T cells.

In addition, co-cultures were prepared. Adherent target cells were plated one day before setting up the co-culture. For the Incucyte®-based cytotoxicity assay, target cells were plated in 100 μL of their respective medium in 96 well flat bottom plates at 5 E3 cells per well (647v, A375, SKMEL5) or 7 E3 live cells per well (Hs695T) to achieve a target cell density of ˜1 E4 cells per well after 20-24 hours incubation at 37° C., 5% CO₂. Seeding densities were adapted according to the variable growth rates of cell lines. For cytokine and proliferation assays, target cells were plated in 96 well flat bottom plates at 2.5 E4 live cells per well (647v, A375, SKMEL5S) or 3.5 E4 (Hs695T) live cells per well to achieve a target cell density of ˜5 E4 live cells per well after 20-24 h incubation. Non-adherent cells (i.e., T2) were plated the day of initiation of the co-culture using assay-specific seeding densities.

The following discussion further details certain experiments and results introduced above.

a. Flow Cytometric Analysis of Process-Representative TSC-203-A0201 TCR-T Cells.

First, the cellular composition of TSC-203-A0201 TCR-T cells was examined by flow cytometry. Effectors were thawed as described above. After overnight recovery, TCR-T cell test articles and their untransfected controls were washed, labeled with antibodies from the “Dextramer Panel” (Table 8) and acquired. Unstained cells, single stain controls and FMO controls were also prepared.

TABLE 8
Cancer cell, media, flow cytometry “Dextramer Panel”, and T cell proliferation
detection reagents, and appendix reagents, respectively
Cancer cell reagents

| | | | Catalog | PRAME | HLA-A*02:01 |
|---|---|---|---|---|---|
| Target cell line | Indication | Source | number | (TPM) | (HLA-A TPM) |
| 647v | Bladder urothelial | DSMZ* | ACC414 | − | + |
| | carcinoma | | | (0.1) | (988) |
| A375 | melanoma | ATCC | CRL-1619 | + | + |
| | | | | (130) | (219) |
| Hs695T | melanoma | ATCC | HTB-137 | + | + |
| | | | | (242) | (521) |
| SKMEL5 | melanoma | ATCC | HTB-70 | + | + |
| | | | | (237) | (380) |
| T2 | Lymphoblast | ATCC | CRL-1992 | − | + |

Media reagents

| Media | Components | V stock (mL) | Catalog Number |
|---|---|---|---|
| Cytokine-free T cell | X-VIVO 15 | 1000 | Lonza |
| medium | | | #04-418Q |
| | Human Serum (Heat- | 50 | Sigma-Aldrich |
| | Inactivated) | | #H3667-100 |
| | Penicilline Streptomycine | 10 | ThermoFisher Scientific |
| | | | #15149-122 |
| | GlutaMAX (100x) | 10 | ThermoFisher |
| | | | Scientific |
| | | | #35050061 |
| | Total | 1070 | n/a |
| Complete T cell | Cytokine-free T cell media | 200 | n/a |
| medium | Human IL-2 (5 μg/mL) | 1 | Sigma-Aldrich |
| | | | #11147528001 |
| | Human IL-7 (10 μg/mL)* | 0.1 | R&D Systems |
| | | | #207-IL-025 |
| | Total | 201.1 | n/a |
| 647v Culture Medium | DMEM | 500 | ATCC |
| (DMEM based | | | #11965-092 |
| medium) | Fetal Bovine serum | 75 | ThermoFisher Scientific |
| | | | #15149-122 |
| | Penicillin-Streptomycin | 5 | Thermofisher Scientific |
| | | | #A3840001 |
| | Total | 580 | n/a |
| Culture medium for | EMEM | 500 | ATCC |
| Hs695T and SKMEL5 | | | #30-2003 |
| (EMEM based | Fetal Bovine serum | 50 | ThermoFisher Scientific |
| medium) | | | #15149-122 |
| | Penicillin-Streptomycin | 5 | Thermofisher Scientific |
| | | | #A3840001 |
| | Total | 555 | |
| T2 Cell Culture | IMDM | 500 | ATCC #30-2005 |
| Medium (IMDM based | Fetal Bovine Serum | 100 | Thermofisher Scientific |
| medium) | | | #A3840001 |
| | Penicillin/Streptomycin | 5 | Thermofisher Scientific |
| | | | #15149-122 |
| | Total | 605 | n/a |
| Serum-Free IMDM | IMDM | 500 | ATCC #30-2005 |
| based medium | | | |
| (Pulsing T2 cells) | | | |
| T2 cell medium for | RPMI1640 | 500 | |
| cocultures testing | Fetal Bovine Serum | 50 | Thermofisher Scientific |
| TGFb resistance | | | #A3840001 |
| (RPMI-based | Penicillin-Streptomycin | 5 | Thermofisher Scientific |
| medium) | | | #15149-122 |
| | Total | 555 | n/a |
| Serum-free | RPMI1640 | 500 | ATCC |
| RPMI1640 (for | | | #30-2001 |
| peptide pulsing of T2 | Penicillin-Streptomycin | 5 | Thermofisher Scientific |
| for TGFb resistance | | | #15149-122 |
| assay) | Total | 505 | n/a |

Flow cytometry “Dextramer panel” reagents

| Antibody | Clone | Vendor | Fluorochrome | Catalog Number |
|---|---|---|---|---|
| near-IR fluorescent | N/A | Thermofishe | Near-IR | L34976A |
| reactive dye | | Scientific | (APC-Cy7) | |
| CD34 | QBEND/10 | Thermofisher | Biotinylated | MA5-16924 |
| | | Scientific | | |
| Streptavidin | N/A | BioLegend | AF488 | 40235 |
| CD4 | RPA-T4 | BioLegend | PE-Cy7 | 300538 |
| CD8b | 2ST8.5H7 | BioLegend | BV421 | 742390 |
| TGFβRII | W17055 | BioLegend | PE | 399704 |
| PRAME/HLA-A*02:01 | PRAME (425- | IMMUDEX | APC | Custom (HLA-A*0201/ |
| Dextramer | 433) | | | SLLQHLIGL) |

Peptide-Pluse reagents

| | Peptide | Gross | | | |
|---|---|---|---|---|---|
| Peptide | Sequence | Weight | Purity | Vendor | Catalog Number |
| PRAME | SLLQHLIGL | 2.0 mg | 91.0%, | GenScript | U4197ZPNG0-1 |
| (425-433) | (425-433) | | | | |

T cell proliferation detection reagents

| | | | | Catalog |
|---|---|---|---|---|
| Reagent | Clone | Vendor | Fluorochrome | Number |
| Far Red Fixable Live-Dead | N/A | Thermofisher | APC equivalent | L34974 |
| Dye | | Scientific | | |
| CD34 | QBEND/10 | R&D Systems | Alexa Fluor 488 | FAB7227G |
| TCR α/β | IP26 | Biolegend | PE-Cy7 | 306720 |
| CD4 | OKT4 | Biolegend | APC-Cy7 | 317418 |
| CD8 | HIT8α | Biolegend | PerCP-Cy5.5 | 300924 |
| CellTrace Violet (CTV) | NA | Thermofisher | PB450 | C34557 |
| | | Scientific | | |

TGFβ mediated inhibition of T cell proliferation dectection reagents

| Reagent for the analysis of | Antibody | | Fluorochrome/ | |
|---|---|---|---|---|
| SKMEL5 cocultures | Clone | Vendor | Channel | Catalog Number |
| Fixable Far Red live/dead | N/A | Thermofisher | APC | L34973 |
| dye | | Scientific | | |
| Fixable Green live/dead | N/A | Thermofisher | FITC | L34970 |
| dye | | Scientific | | |
| CellTrace Violet (CTV) | N/A | Thermofisher | BV421 analog | C34557 |
| | | Scientific | (PB450) | |
| CD34 | QBEND/10 | Thermofisher | N/A | MA5-16924 |
| | | Scientific | | |
| Streptavidin | N/A | Biolegend | Alexa 488 | 405207 |
| Streptavidin | N/A | Biolegend | APC | 405207 |
| TCR α/β | IP26 | Biolegend | PE-Cy7 | 306720 |
| CD4 | OKT4 | Biolegend | APC-Cy7 | 317418 |
| CD8 | HIT8α | Biolegend | PerCP-Cy5.5 | 300924 |

Appendix reagents

| Antibody | Clone | Vendor | Fluorochrome | Catalog Number |
|---|---|---|---|---|
| near-IR fluorescent | N/A | Thermofisher | Near-IR | L34976A |
| reactive dye | | Scientific | (APC-Cy7) | |
| Live/DeadTM Green | N/A | Thermofisher | N/A | L34970 |
| Dye | | scientific | | |
| Live/DeadTM Far Red | N/A | Thermofisher | Far IR | L34973 |
| Dye | | scientific | | |
| CD34 | QBEND/10 | Thermofisher | Biotinylated | MA5-16924 |
| | | Scientific | | |
| Streptavidin | N/A | BioLegend | Alexa488 | 40235 |
| Streptavidin | N/A | BioLegend | APC | 405207 |
| CD4 | RPA-T4 | BioLegend | PE-Cy7 | 300538 |
| CD4 | Okt4 | BioLegend | APC-Cy7 | 317418 |
| CD8b | 2ST8.5H7 | BioLegend | BV421 | 742390 |
| CD8 | Hit8a | Biolegend | Percp-Cy5 | 300924 |
| TGFβRII | W17055 | BioLegend | PE | 399704 |
| PRAME/HLA-A*02:01 | PRAME (425-433) | IMMUDEX | APC | Custom (HLA-A*0201/ |
| Dextramer | | | | SLLQHLIGL) |
| TCRαβ | IP26 | BioLegend | PE-Cy7 | 306720 |
| Count BrightTM | N/A | Thermofisher | N/A | C36950 |
| Absolute counting | | scientific | | |
| beads | | | | |
| UltraComp ebeadsTM | N/A | Thermofisher | N/A | 01-33333-42 |
| plus | | scientific | | |

| | Peptide | Gross | | | |
|---|---|---|---|---|---|
| Peptide | Sequence | Weight | Purity | Vendor | Catalog Number |
| PRAME | SLLQHLIGL | 2.0 mg | 91.0%, | GenScript | U4197ZPNG0-1 |
| (425-433) | (425-433) | | | | |

| | Vendor | Catalog Number |
|---|---|---|
| Media | | |
| IMDM | ATCC | 30-2005 |
| XVIVO 15 | Lonza | 04-418Q |
| DMEM + D-Glucose + L-Glutamine | ATCC | 11965-092 |
| EMEM + L-Glutamine | ATCC | 30-2003 |
| RPMI1640 | ATCC | 30-2001 |
| Human Serum (Heat-Inactivated) | MP CELLect 100 ml | 2930949 |
| Penicilline-Streptomycin | Thermofisher Scientific | 15149-122 |
| 100x Glutamax | Thermofisher Scientific | 35050-061 |
| FBS, heat-inactivated | Thermofisher Scientific | A3840001 |
| Reagent | | |
| DMSO | Sigma Life Science | D2650-100ML |
| DPBS/Modified | Gibco | 14190-144 |
| ViaStain AOPI Staining Solution | Nexcelom Bioscience | CS2-0106-25mL |
| 70% Denatured Ethanol | Decon Laboratories inc | 8601 |
| Poly-L-Ornithine Solution, 0.01% | Millipore-Sigma | P4957 |
| recombinant human IL2 | Sigma-Aldrich | 11147528001 |
| recombinant human IL7 | RD Systems | 207-IL/CF |
| reconstitution buffer 2 (0.1% BSA in PBS) | RD Systems | RB02 |
| EasySep ™ buffer | Stem Cell Technologies | 20144 |
| anhydrous DMSO | Thermofisher Scientific | D12345 |
| CellTraceTM Violet proliferation kit | Thermofisher Scientific | C34557 |
| BD CytofixTM Fixation Buffer | BD Bioscience | 554655 |
| recombinant human TGFb1 | RD Systems | 7754-BH-025/CF |
| reconstitution buffer 4 (HCl-BSA) | RD Systems | RB04 |
| CellTraceTM CFSE proliferation kit | Thermofisher Scientific | C34570 |

Data acquisition was performed on a Cytoflex S. Compensation was performed automatically with CytExpert software. Flow cytometric analysis was performed with FlowJo v7.6.5, and the statistics were exported to Excel 2010 and analyzed.

The following description provides the cell gating strategy. Briefly, cells were gated from the FSC versus SSC dot plot and singlets distinguished from the aggregates using FSC-Area versus FSC-Height plot. Viable cells were identified using the Near-Infrared Live-Dead versus FSC-Area plot. Subpopulations were gated from the viable cells and evaluated, including, CD4⁺ (Helper T cells), CD4⁺/CD8⁺ (Engineered Helper T cells), CD4⁻/CD8⁺ (Cytotoxic T cells), and TGFβRII⁺/CD34⁺ (engineered TCR-T cells expressing DN-TGFβRII). Cells identified as CD34⁻ or CD34⁺ were further evaluated for TGFβRII expression. The mean fluorescent intensity (MFI) was measured for CD34/TGFγRII and CD34⁺/TGFγRII.

Flow cytometric analysis confirmed that TSC-203-A0201 TCR-T cells from three different batches contained engineered helper cells (CD4⁺, now CD4⁺/CD8⁺), residual non-engineered helper T cells (CD4⁺/CD8⁻), and cytotoxic (CD8⁺) T cells. The percentage of non-engineered CD4⁺/CD8⁻ helper T cells ranged from 7.91% to 14.50%. Engineered helper T cells expressed the exogenous CD8αβ co-receptor and were characterized as CD4⁺/CD8⁺. The TSC-203-A0201 TCR-T cell material presented between 18.00% and 38.60% of these engineered helper T cells and the percentage of cytotoxic T cells (CD4⁻/CD8⁺) ranged from 48.55% to 73.85%.

The engineered T cells were confirmed to express the recombinant TCR specific to the PRAME-derived peptide SLLQHLIGL bound to HLA-A*02:01, as revealed by dextramer binding, as well as the CD34 epitope. No dextramer positive or CD34 positive population was detected in untransfected (UTF) T cells from matched donors. The percentage of the dextramer⁺/CD34⁺ population ranged from 37.75% to 42.85%.

Both the engineered CD4⁻ and CD4⁺ T cells were able to bind the dextramer, confirming that functional TCRs were found on the surface of both the helper and cytotoxic T cells. The percentage of Dextramer⁺/CD4⁻ populations ranged from 22.10% to 38.50%, and the percentage of Dextramer⁺/CD4⁺ ranged from 4.79% to 11.55%.

Engineered T cells making up TSC-203-A0201 material also expressed the DN-TGFβRII, as evidenced by a marked increase in the TGFβRII⁺ signal in the CD34⁺ population when compared to non-engineered CD34⁻ cells.

b. pMHC Dose-Dependent Function of the Recombinant TCR Expressed by TSC-203-A0201 TCR-T Cells

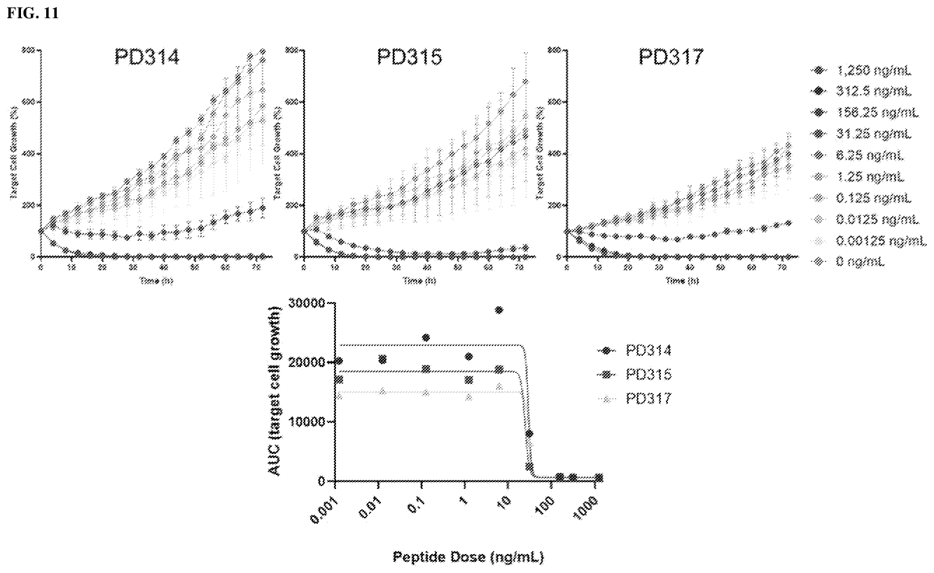

FIG. 11 shows reactivity of TSC-203-A0201 TCR-T cells from three independent donors against their cognate peptide/MHC as determined using a peptide pulse assay. NucLightRed-transduced T2 target cells were peptide-pulsed with a titration of the PRAME peptide SLLQHLIGL and co-cultured with TSC-203-A0201 TCR-T cells. TCR reactivity was assessed by measuring the growth of the target NucLightRed-transduced T2 cells.

Specifically, effectors were thawed as described above. On the day of co-culture, T cells were harvested, washed once with cytokine-free T cell medium, and resuspended in cytokine-free T cell medium. Cell density was adjusted to 2.0 E5 live cell/mL with cytokine-free T cell medium. On the day of co-culture, T2 cells were harvested and washed once with serum-free IMDM based medium. The cell density was adjusted to 4.0 E6 live cells/mL and 2 mL (8.0 E6 live cells) were transferred to separate vessels to be pulsed with the peptides. T2 target cells were pulsed with a titration of the HLA-A*02:01-restricted PRAME-derived peptide (SLLQHLIGL). Titration of peptides were prepared in serum-free IMDM based medium; 15-point, ranging from 25,000,000 pg/mL to 0.0125 pg/mL. The target cells were resuspended with 2.0 mL of the diluted peptide for a final concentration of 2.0 E6 live cells/mL and incubated in a humidified incubator at 37° C., and 5% CO₂ for 45 mins to 2 hours. Peptide-pulsed target cells were then washed three times with T2 Cell Culture medium. The cell density was adjusted to 1.0 E5 live cells/mL. Prepared peptide pulsed T2 cells were resuspended and 100 μL (1.0 E4 cells total) were added per a well. Prepared TCR-T cells were resuspended and 100 μL (2.0 E4 cells total) were added on top of the pulsed target cells for a final E:T ratio of 2:1. For the target cells alone condition, cytokine-free T cell media (100 μL) was added on top of the plated target cells. The combined target and effector cells were allowed to settle for ˜20 minutes in a humidified incubator at 37° C., and 5% CO₂, prior to the initiation of the acquisition.

Samples were acquired in the IncuCyte® S3 Live-Cell Analysis system (Sartorius, Goettingen, Germany). IncuCyte® acquired data was analyzed using IncuCyte® S3 Live-Cell Analysis system software GUI Version 2021A (Sartorius, Goettingen, Germany). A customized analysis definition has been created to quantify the area occupied by red objects (i.e., Nuclight Red-positive T2 cells) over time.

Target cell growth was measured in Red Channel Areas Per Image (4 per well) and normalized to 0d0h0m. The raw data was exported and graphed in GraphPad Prism (v5.02). Time in hours was plotted on the x-axis and target cell growth plotted as a percentage on the Y-axis. The area under the curve (AUC) of the T2 cell growth for each peptide dose was calculated using GraphPad Prism software. The AUCs were plotted (y-axis) as a function of peptide dose (x-axis) and a nonlinear regression curve (using the equation Sigmoidal, 4 PL, X is concentration) was used to graph the dose-dependent cytotoxicity of the test articles.

As shown in FIG. 11, the three batches of process-representative TSC-203-A0201 TCR-T cells displayed a dose-dependent cytotoxic activity toward T2 pulsed with the PRAME-derived peptide SLLQHLIGL and cytotoxicity was observed at 31.25 ng/mL and saturated at ˜156.25 ng/mL. Altogether, these data confirmed that TSC-203-A0201 TCR-T cells are highly specific and efficiently react to the PRAME-derived peptide SLLQHLIGL presented on HLA-A*02:01.

c. TSC-203-A0201 TCR-T Cells Exhibit Target-Dependent Cytokine and Granzyme B Production

Target-dependent cytokine induction and granzyme B secretion of TSC-203-A0201 TCR-T cells were evaluated. Target-dependent induction of granzyme B secretion, as well as secretion of the pro-inflammatory cytokines IFN-γ, IL-2 and TNF-α, was assessed after ˜24 hours co-culture of the three batches of TSC-203-A0201 TCR-T cells with a panel of cancer cell lines selected for their endogenously expressing PRAME and HLA-A*02:01. 647v cells, which are positive for HLA-A*02:01 but negative for PRAME, as well as donor-matched untransfected (UTF) control T cells were included as negative controls in the experiment. A ‘T cell only’ condition wherein TSC-203-A0201 TCR-T cells or UTF control T cells were cultured in the absence of any target cells was used to establish baseline levels of Granzyme B, IFN-γ, IL-2 and TNF-α in the assay.

Specifically, adherent target cells were plated one day before setting up the co-culture, as described above, and effectors were prepared for the co-culture as described above. To initiate the coculture. TSC-203-A0201 TCR-T cells and UTF controls were diluted to 5 E5 live cells/mL in cytokine-free T cell medium and 100 μL of the cell solution (corresponding to 5 E4 live T cells) were added to the target cells, resulting in an E:T of 1:1. As a control, both effector alone and target alone wells were prepared; these controls received 100 μL of DMEM 10% FBS 1% PS or 100 μL of cytokine-free T cell medium, respectively. After 24 hours of coculture at 37° C., 5% CO₂, supernatants were collected and stored at −80° C. Subsequently, IFN-γ, TNF-α, IL-2 and Granzyme B secreted into the supernatant was quantified with an automated 4-plex ELISA platform (ELLA from Protein Simple ELLA) according to the manufacturer's instructions. Simplex Explorer software was used to run samples on the ELLA. Raw data were exported as excel file and the concentration was calculated by multiplying the reported cytokine concentration with the dilution factor. Data were then plotted in Graphpad Prism (v9.5.1).

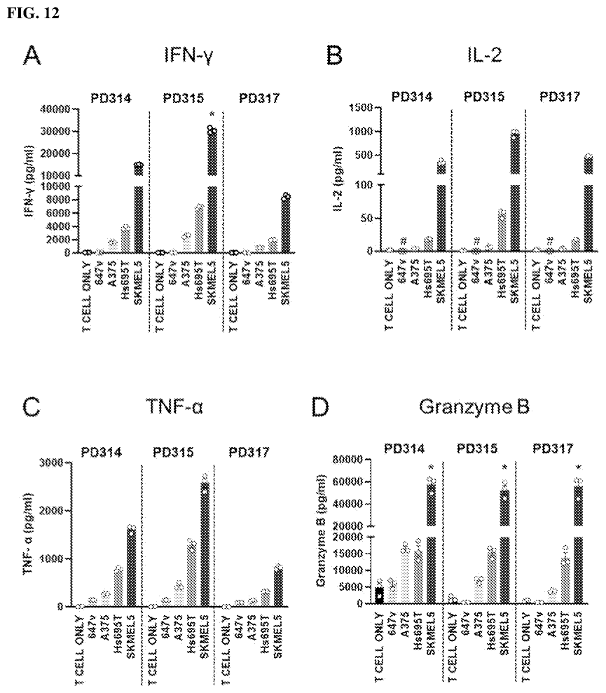

FIG. 12A-FIG. 12C show that when TSC-203-A0201 TCR-T cells were cultured alone, TCR-T cells only secreted baseline levels of IFN-γ (ranging from 21 to 59 pg/mL), IL-2 (1.2 to 2.3 pg/mL) and TNF-α (4 to 11 pg/mL). Similar baseline levels were observed with UTF control T cells. Furthermore, when TSC-203-A0201 TCR-T cells were co-cultured with the negative control cell line 647v, the levels of production of IFN-γ and IL-2 were similar to the baseline levels observed in the ‘T cell only’ condition, while only a modest induction of TNF-α secretion was observed. Importantly, this low level of induction of TNF-α was also observed with UTF control T cells, demonstrating that this induction was not mediated by the therapeutic TCR (FIG. 12G).

Upon coculture of TSC-203-A0201 TCR-T cells with Hs695T and SKMEL5 cell lines which express both PRAME and HLA-A*02:01, a robust induction of secretion of IFN-γ, TNF-α and IL-2 was observed for all batches of TSC-203-A0201 TCR-T cells tested. When compared to ‘T cell only’ condition, TSC-203-A0201 presented a 64-1440-fold induction of IFN-γ, 8-795-fold induction of IL2 and ˜69-416-fold induction of TNF-α in the presence of the Hs695T (medium blue bars) and SKMEL5 (dark blue bars) cell lines. Importantly, the induction of TNF-α upon coculture with the PRAME-positive cell lines Hs695T and SKMEL5 was much higher than upon coculture with the PRAME-negative control cell line 647v (69-417-fold compared to ˜12-22-fold, respectively; see FIG. 12C-FIG. 12G. Less cytokine secretion was observed when TSC-203-A0201 TCR-T cells were cocultured with the A375 cell line (FIG. 12A-FIG. 12C). For this cell line, IFN-γ induction ranged from 25-114-fold, IL-2 induction from ˜2-5-fold and TNF-α induction from ˜24-70-fold.

Similar to the trends observed for pro-inflammatory cytokines upon coculture with the PRAME-negative and HLA-A*02:01-positive cell line 647v, all batches of TSC-203-A0201 TCR-T cells tested secreted only baselines levels of Granzyme B upon coculture with 647v, (FIG. 12DD). These data demonstrate lack of reactivity of TSC-203-A0201 TCR-T cells to HLA-A*02:01-positive target cells in the absence of expression of the target protein PRAME. Upon co-culture with PRAME⁻ and HLA-A*02:01-positive target cell lines, an increase in secretion of Granzyme B was observed with all batches of TSC-203-A0201 TCR-T cells tested (FIG. 12D). In comparison to the ‘T cells only’ condition, cocultures with A375. Hs695T and SKMEL5 induced a ˜3.4-4.9-fold, ˜3.2-15-fold and ˜11.8-60-fold increase of Granzyme B secretion in TSC-203-A0201 TCR-T cells. Overall, these data indicate that TSC-203-A0201 TCR-T cells demonstrate a target-dependent induction of secretion of Granzyme B. UTF control T cells displayed no or limited induction of Granzyme B response upon co-culture with the positive and negative target cells lines relative to the ‘T cell only’ condition indicating lack of activation of donor T cells in the absence of the therapeutic TCR (FIG. 12H).

Taken together, these data demonstrate that TSC-203-A0201 TCR-T cells operate in a target-dependent manner, reacting to target cell lines endogenously expressing PRAME and HLA-A*02:01 by secreting pro-inflammatory cytokines and Granzyme B.

d. TSC-203-A0201 TCR-T Cells Proliferate in a Target-Dependent Manner

Target-dependent proliferation of the engineered T cells (both CD4+ and CD4−) was also assessed. Briefly, effectors were thawed and allowed to recover overnight as described above. To eliminate baseline proliferation induced by the T cell cytokines IL-2 and IL-7, effectors were washed once with cytokine-free T cell medium, reseeded in 6 well G-REX® plates at a concentration of 1-2 E6 live cell/mL and incubated for an additional 20-24 hours in cytokine-free T cell medium. Adherent target cells (647v, A375, Hs695T and SKMEL5) were plated one day before setting up the co-culture as described above. Before initiating the co-culture, effectors were stained with cell trace violet (CTV) dye as follows: effectors were washed once with EasySep™ and stained for 7 minutes at room temperature with CTV dye (reconstituted according to manufacturer's instructions and diluted 1:2000 in EasySep™ buffer). After washing T cells twice with cytokine-free T cell medium, the T cell concentration was adjusted to 5 E5 live cells/mL and 5 E4 CTV labeled T effectors in 100 μL of cytokine-free T cell medium were added to the target cells to achieve an E:T of 1:1. As a control, both effector alone and target alone wells were prepared; these controls received 100 μL of DMEM 10% FBS 1% PS or 100 μL of cytokine-free T cell medium, respectively. Note that to ensure that each well contained enough T cells for evaluation of T cell proliferation, 6 replicates were set up for each condition, and 2 replicates were pooled at the end of the coculture for the staining procedure. To minimize evaporation, plates were covered with BreathEasy membrane. Target and effectors were then co-cultured for 3.5 days at 37° C., 5% CO₂. At the end of the co-culture, effectors were transferred to a v-bottom plate and stained with the staining reagents (see Table 8 above), followed by fixation with Cytofix (BD Bioscience). After washing out the fixative, samples were resuspended in 100 μL Easysep™ buffer, and 10 μL of Absolute CountBright counting beads (Thermofisher Scientific) were added to the wells immediately before acquisition.

Samples were acquired on a Cyoflex S (Beckman Coulter) using Cytexpert Software. Data were analyzed with Flowjo v10.8.1, and the gating strategy is depicted below. Briefly, for transduced T cells, the number of proliferating TCRαβ⁺CD34⁺ (total transduced T cells), proliferating TCRαβ⁺CD34⁺ CD4⁺CD8⁺ (transduced helper T cells) and proliferating CD34⁺CD4⁻CD8⁺ T cells (transduced cytotoxic T cells) was determined. For untransduced control T cells, the number of proliferating TCRαβ⁺ T cells (total T cells), proliferating TCRαβ⁺CD4⁺ (helper T cells) and proliferating TCRαβ⁺CD8⁺ (cytotoxic T cells) was determined. Raw data were exported as excel files, and the number of proliferating cells was normalized to the bead count. Normalized cell counts were then graphed in GraphPad Prism (v9.5.01).

The following provides the cell gating strategy used. Briefly, absolute counting beads, which are SSC-A high, were first separately gated out from the cells in the FSC-A vs SSC-A plot. Using the same plot, cells were separated from debris. After gating on cells, single cells were separated from aggregates using the FSC-Height (FSC-H) versus FSC-A plot. The target cells used for the coculture were engineered to express Nuclight® Red, a fluorescent protein with a broad emission spectrum that is detectable in the APC channel. Furthermore, dead cells were stained with an APC analog (fixable far red live dead dye). Therefore, both target cells and dead cells were gated out as APC⁺ events in an APC versus FSC-A plot. In the target alone controls, it was apparent that a small fraction of target cells appeared to be APC negative, likely due to loss of expression of the NLR protein. Furthermore, possibly due to autofluorescence, these target events were not eliminated when gating on TCRαβ⁺CTV⁺ events (not shown). However, it was possible to efficiently gate out these residual target events by drawing a gate encompassing CD4⁺ and CD8⁺ events. Finally, to further identify T cells, a gate was drawn on TCRαβ⁺CTV⁺ events (note that T cells are CTV⁺ due to CTV labeling prior to the initiation of the coculture). Subsequently, transduced T cells were gated as follows. Transduced (i.e., CD34⁺) T cells were identified in a CD34 versus FSC-A plot and were further separated into helper T cells (CD4⁺) and cytotoxic T cells (CD8⁺) in a CD4 versus CD8 plot. Note that because engineered CD4+ T cells express exogenous CD8αβ proteins. CD4⁺CD8⁺ double positive helper T cells were distinguished from CD8⁺ single positive cytotoxic T cells based on CD4 expression. Finally, dilution of the Cell Trace Violet dye was assessed in the total TSC-203-A0201 engineered T cell population (CD34⁺) as well as in the helper (CD34⁺CD8⁺CD4⁺), and cytotoxic (CD34⁺ CD8⁺CD4⁻) T cell subsets using TCRαβ versus CTV plots. As the UTF control T cells did not undergo genetic modification and lacked the CD34 marker as well as exogenously expressed CD8αβ, UTF controls were gated differently: proliferating cells (i.e., CTV dim cells) were identified within the total T cell population (TCRαβ⁺), and helper and cytotoxic T cell population were distinguished on the basis of CD4 (TCRαβ⁺CD4⁺) and CD8 (TCRαβ⁺CD8⁺) positivity, respectively. TCR-T cells cultured in the absence of any targets (‘T cell only’) or in the presence of the HLA-A*02:01 positive PRAME negative control cell line 647v were used to identify the position of the CTV peak of undivided cells and assess baseline levels of proliferation.

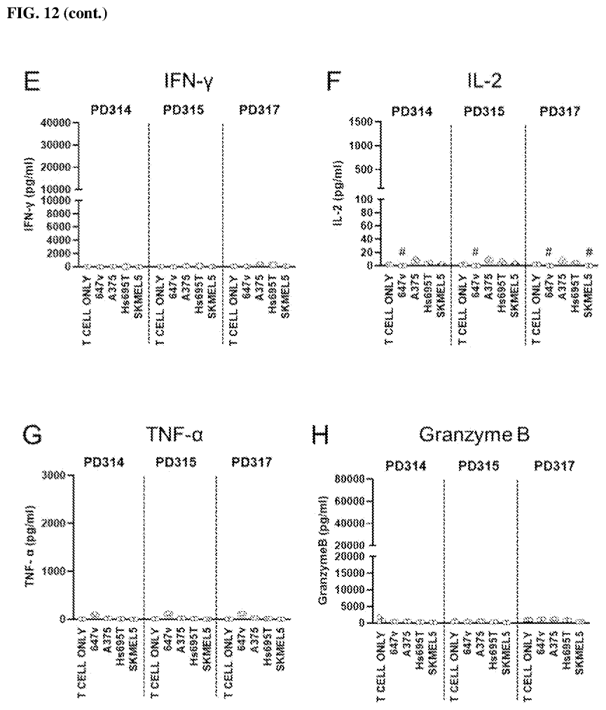

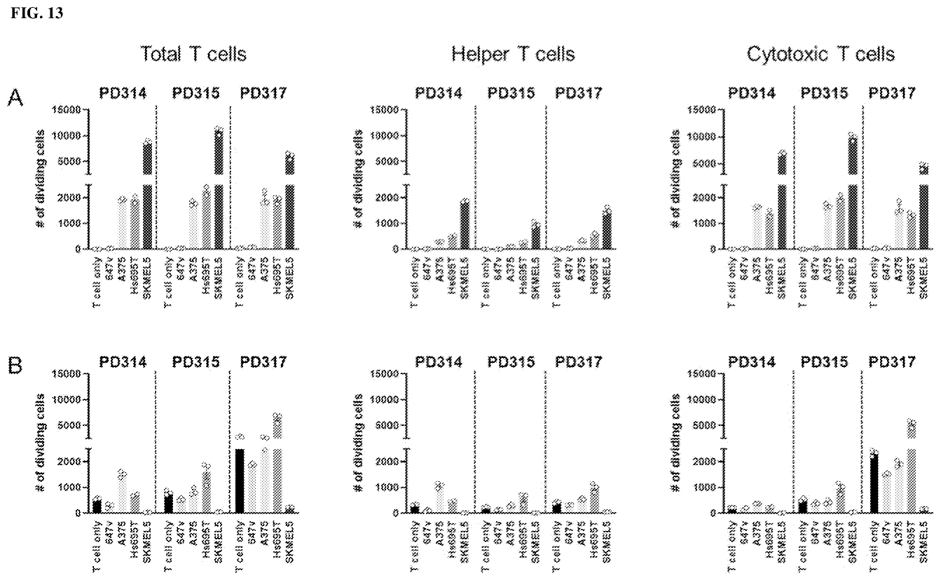

FIG. 13 shows results of evaluation of target-dependent induction of proliferation. The panel of cancer cell lines used to evaluate target-dependent induction of cytokine and granzyme B secretion was also used to assess target-dependent induction of T cell proliferation. The three independent batches of process-representative TSC-203-A0201 TCR-T cells were labeled with CTV dye and cultured alone, or co-cultured with the cancer cells for ˜3.5 days at an E:T of 1:1. Subsequently, proliferation was evaluated as described above.

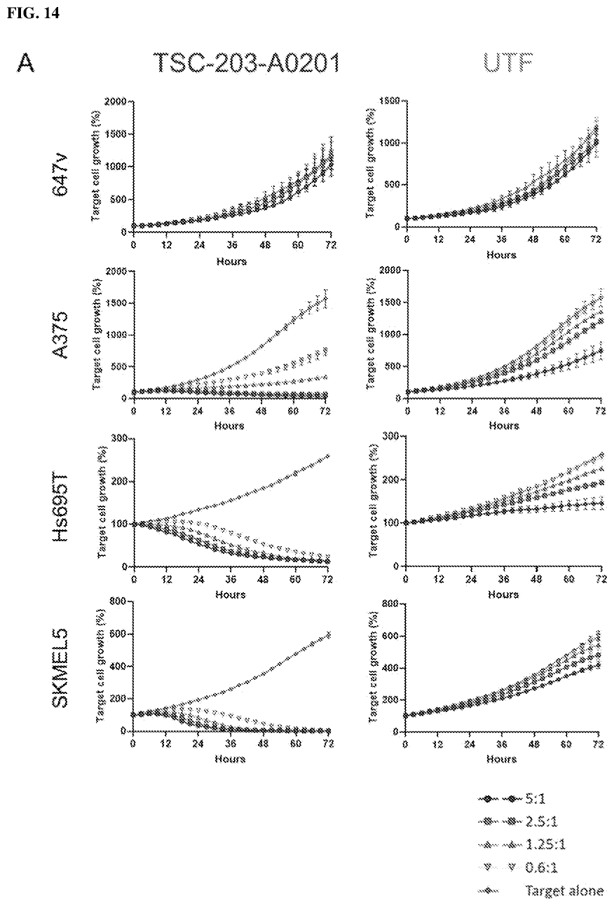

When cultured in the absence of target cells (‘T cell only’), TSC-203-A0201 TCR-T cells did not proliferate (FIG. 13A), and only a modest increase in the number of proliferating cells was observed when TSC-203-A0201 TCR-T were cocultured with the PRAME negative control cell line 647v. Importantly, coculture with 647v cells did not elicit meaningful cytokine secretion or a cytotoxic response in TSC-203-A0201 TCR-T cells (FIG. 12 and FIG. 14), indicating that the low levels of proliferation were not due to T cell activation. Taken together, these data demonstrate a lack of reactivity of TSC-203-A0201 TCR-T cells to HLA-A*02:01-positive target cells in the absence of PRAME protein.

On the other hand, coculture of TSC-203-A0201 TCR-T cells with three target cell lines expressing PRAME and HLA-A*02:01 (i.e., A375, Hs695T and SKMEL5) induced a robust proliferation of the TCR-T cells across all three batches of TSC-203-A0201 (FIG. 13Error! Reference source not found.A). Both helper and cytotoxic TCR-T cells mounted a proliferative response upon coculture with the PRAME-positive, HLA-A*02:01-positive target cells. However, more proliferation was occurring in the engineered cytotoxic T cell population than in the helper T cell population. Accordingly, the absolute numbers of dividing cytotoxic TCR-T cells were in most cases 2-3-fold higher than those observed for helper TCR-T cells (FIG. 13A). The proliferation of TSC-203-A0201 was strongest when challenged with SKMEL5 (dark blue bars), followed by Hs695T (medium blue bars) and weakest when cocultured with A375 (light blue bars), see FIG. 13A. It is noteworthy that the proliferative response of TSC-203-A0201 appeared to correlate with the levels of IL-2 produced in the different cocultures. SKMEL5 elicited both the strongest proliferative response, and the highest levels of secretion of the T cell mitogen IL-2, which is needed for the T cells' proliferation and survival (FIG. 12B).

As additional controls. UTF control T cells displayed overall a high baseline of proliferation when compared to TSC-203-A0201 TCR-T cells (compare ‘T cell alone’ conditions in FIG. 13A and FIG. 13B). Nevertheless, the proliferation of UTF control T cells did not increase when cocultured with the 647v and SKMEL5 cancer cell lines. However, UTF control cells exhibited a weak donor-dependent proliferative response when cocultured with A375 or Hs695T cells: a modest proliferation of the control cells from PD315 and PD317 (mostly cytotoxic T cells) was observed when cocultured with Hs695T, and from control cells from PD314 (mostly helper T cells) when cocultured with A375. Since UTF control cells secreted also low levels of cytokines in response to these cell lines, it is likely that alloreactivity mediated by the endogenous TCR of the UTF control cells underlies the weak proliferative response (FIG. 12). However, the magnitude of cell proliferation was much lower for UTF control cells than what was measured when TSC-203-A0201 TCR-T cells were cocultured with the PRAME-positive, HLA-A*02:01-positive target cell lines (i.e. ˜2-3 fold versus ˜30-800-fold, respectively), demonstrating the lack of significant T cell activation in response to these cell lines in the absence of the therapeutic TCR.

Taken together, these data show that TSC-203-A0201 TCR-T cells are capable of mounting a robust proliferative response when encountering target cells that express PRAME and HLA-A*02:01.

e. TSC-203-A0201 TCR-T Cells Exhibit Selective and Potent Cytotoxic Function

To assess the cytotoxicity potential of TSC-203-A0201 TCR-T cells, three independent batches of process-representative TSC-203-A0201 TCR-T cells were tested in an IncuCyte®-based cytotoxicity assay. The effector T cells were serially diluted and cocultured with a fixed number of target cells to test different effector to target ratios (E:T). Untransfected (UTF) control T cells from matching donors were similarly tested as negative controls. The target cell lines tested corresponded to the panel also used to test induction of cytokine secretion and proliferation. The panel included PRAME-positive, HLA-A*02:01-positive cell lines (i.e., A375. Hs695T and SKMEL5) as well as a negative control target cell line (i.e., 647v, which is HLA-A*02:01 positive, but negative for PRAME). The target cells were engineered to express Nuclight Red, a fluorescent protein enabling the tracking and quantification of cell growth over time in the IncuCyte® imager.

Specifically, targets were engineered to express the fluorescent protein NuclightRed (NucLightRed, Essen Bioscience) following the manufacturer's instruction to allow tracking of target cells in the IncuCyte®-based cytotoxicity assay. Target cells were plated the day before initiating the co-culture as described above. Process-representative test articles and their untransfected (UTF) donor-matched controls were thawed and rested overnight as described above. Serial dilutions of effectors (TSC-203-A0201 TCR-T cells or donor-matched UTF cells) were prepared in cytokine-free T cell medium to obtain the plating concentrations and 100 μL of effectors were added to the targets, resulting in an E:T titration ranging from 5:1 to 0.6:1. For the target cell only condition 100 μL of cytokine-free T cell medium was added to the target cells. Plates were sealed with BreathEasy membrane to limit evaporation of medium and were allowed to settle at room temperature for 10-15 minutes. After an additional 15 minutes incubation at 37° C., 5% CO₂, a kimwipe was used to wipe off condensation of the bottom of the plates and acquisition was started. Samples were acquired on an Incucyte® system as described above. Raw data were exported as excel sheet and the target cell growth normalized to the 0 hour time point was plotted in Graphpad Prism (v v9.5). The area under the curve (AUC) was calculated for the growth curves obtained with target cells cocultured with TSC-203-A0201 and UTF control TCR-T cells at an E:T of 2.5:1, and the AUC of TSC-203-A0201 was normalized to the corresponding UTF control.

Representative growth curves of target cells cocultured for 3 days with TCR-T cells from batch PD315 and donor-matched UTF control cells are shown in FIG. 14A; the area under the curve (AUC) for growth curves obtained with TSC-203-A0201 TCR-T cells from all three batches at an E:T of 2.5:1, normalized to the corresponding UTF control TCR-T cells, are shown in FIG. 14B.

UTF control cells showed some inhibition of target cells, which appeared to be donor and E:T dependent. In contrast to UTF control T cells. TSC-203-A0201 TCR-T cells displayed a dose-dependent and selective killing response, leading to inhibition of the target cell growth upon coculture with the PRAME-positive and HLA-A*02:01 positive cell lines A375. Hs695T and SKMEL5, while little to no growth inhibition was observed when TSC-203-A0201 TCR-T cells were cocultured with the HLA-A*02:01 positive and PRAME negative cell line 647v (FIG. 14).

Taken together, these data demonstrate that TSC-203-A0201 TCR-T cells are capable of mounting a potent and selective cytotoxic response, sparing PRAME-negative target cells, but efficiently targeting and killing PRAME-positive cancer cells.

f. TSC-203-A0201 TCR-T Cells Exhibit Resistance to TGFbeta Signaling

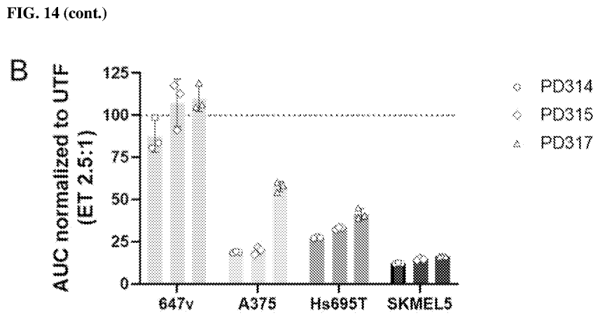

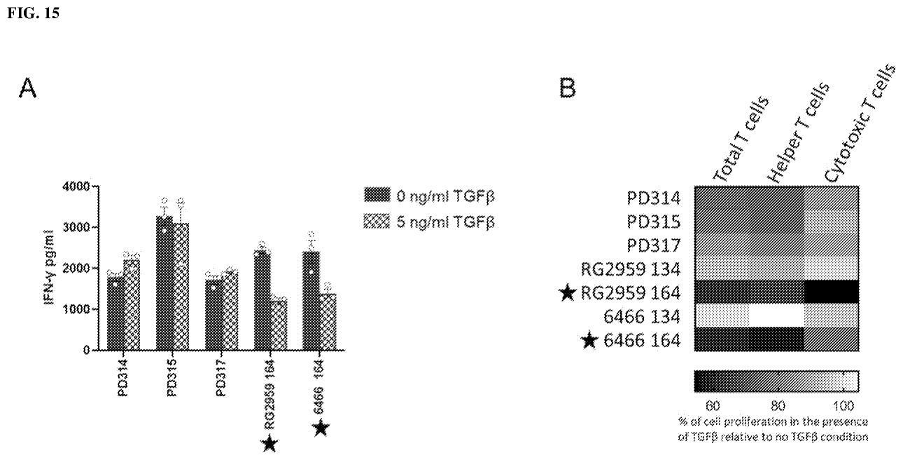

FIG. 15 show the ability of the engineered TSC-203-A0201 TCR-T cells to resist TGFβ-mediated inhibition of T cell function. To demonstrate the ability of TSC-203-A0201 TCR-T cells to resist TGFβ signaling, the target-dependent induction of IFN-γ secretion and of cell proliferation of process-representative TCR-T cells in the presence of physiological levels of TGFβ were tested. Briefly, T2 cells were pulsed with the PRAME-derived peptide SLLQHLIGL and cocultured with 3 batches of process-representative TSC-203-A0201 TCR-T cells in the presence or absence of physiological levels of TGFβ (5 ng/mL. Djurovic et al., 1997; Grainger et al., 1995). Subsequently, the ability of TSC-203-A0201 TCR-T cells to produce IFN-γ in the presence of TGFβ was evaluated. In addition, the proliferative response of TSC-203-A0201 TCR-T cells that were cocultured with either peptide pulsed T2 cells, or the HLA-A*02:01 positive PRAME positive cell line SKMEL5 were tested in the presence or absence of 5 ng/mL TGFβ. As a control for TGFβ-mediated T cell inhibition, two batches of TCR-T cells were engineered with TSC-203-A0201 using either the clinical delivery vector (condition with the suffix “134” in FIG. 15) or a version of the vector lacking the DN-TGFβRII element (condition with the suffix “164” in FIG. 15). This “process-similar” material was generated with a bench scale adaptation of the clinical manufacturing process.

In more detail, the ability of TCR-T cells to produce IFN-γ when cocultured with peptide-pulsed T2 cells in the presence or absence of TGFβ was first tested.

Specifically. SKMEL5 and T2 cells were prepared as follows. Nuclight Red labeled SKMEL5 were plated the day before initiating the co-culture as described above. Parental T2 cells (i.e., non Nuclight Red labeled) were CFSE labeled, and peptide pulsed for the coculture as follows: T2 cells were washed once with cold EasySep™ buffer and were stained for 7 minutes at room temperature with CFSE (reconstituted according to instructions of the manufacturer and diluted 1:1000 in EasySep™ buffer). After washing twice with RPMI1640 10% FBS 1% PS, T2 cells were washed once with serum-free RPMI1640 and were resuspended at 2 E6 live cells/mL in peptide solution (PRAME peptide SLLQHLIGL diluted to 0, 10 or 100 ng/mL in serum-free RPMI1640). After pulsing T2 cells for 45 minutes at 37° C., 5% CO₂ with peptide, CFSE-labeled T2 cells were washed three times with RPMI1640 10% FBS 1% PS. For plating, T2 cells were then resuspended at an assay specific concentration. Note that although T2 cells were maintained in IMDM 20% FBS 1% PS. RPMI1640 10% FBS 1% PS was used for peptide pulsing, plating and coculturing.

To assess IFN-γ secretion, effectors were thawed and allowed to recover in T cell medium with IL2 and IL7 as described above. The next day, effectors were plated as follows: 5 E4 effectors per well were plated in 50 μL cytokine-free T cell medium in a 96 well round bottom plate and 50 μL of 0 or 20 ng/mL TGFβ1 diluted in T cell medium was added for a final concentration of 0 or 10 ng/mL TGFβ1. After 24 hours incubation at 37° C., 5% CO₂+/−TGFβ, 5 E4 CFSE labeled peptide pulsed or unpulsed T2 cells in 100 μL medium or 100 μL of RPMI1640 10% FBS 1% PS (for effector alone control) was added to the effectors, for a final concentration of TGFβ of 0 or 5 ng/mL, and an E:T of 1:1. After 24 hours co-culture, the supernatant was collected and IFN-γ in the supernatant was quantified with an automated ELISA machine (ELLA from ProteinSimple) using IFN-γ ELLA cartridges according to instructions of the manufacturer. Simplex Explorer software was used to run samples on the ELLA. Raw data were exported as excel file and the concentration was calculated by multiplying the reported cytokine concentration with the dilution factor. Data were then plotted in Graphpad Prism (v9.5.1).

FIG. 15A shows that in the absence of TGFβ, two batches of process-similar TCR-T cells lacking DN-TGFβRII, and all three batches of process-representative TCR-T cells produced robust levels of IFN-γ in response to peptide pulsed T2 cells, while little to no IFN-γ was detected when TCR-T cells were cocultured with unpulsed T2 cells or in the absence of T2 cells (data not shown). When a physiological dose of TGFβ (5 ng/mL) was added to the cocultures, the production of IFN-γ by process-similar TCR-T cells lacking DN-TGFγRII decreased by ˜50%. On the other hand, the IFN-γ secretion from TCR-T cells expressing DN-TGFβRII was not affected. Here, all three batches of process-representative TCR-T cells secreted similar levels of IFN-γ, irrespective of whether TGFβ was added to the cocultures or not.

Next, the ability of TCR-T cells to resist TGFβ-mediated inhibition of proliferation was tested.

To assess proliferation, effectors were thawed and allowed to recover in T cell medium with IL2 and IL7 as described above. After overnight recovery from thaw, T cells were starved of cytokines for 20-24 hours and labeled with CTV dye as described above. For coculture with CFSE labeled and peptide pulsed T2 cells. T cells were diluted to 5 E5 or 1 E6 cells/mL in cytokine-free T cell medium, and 50 μL (corresponding to 2.5 E4 or 5 E4 T cells) were plated in a 96 well round bottom plate; 50 μL of 0 or 20 ng/mL TGFβ (diluted in T cell medium) was added. Pulsed or unpulsed CFSE-labeled T2 cells were diluted to 1 E6 or SE5 cells/mL, and 100 μL were added to the T cells, resulting in E:Ts of 0.25:1, 0.5:1 or 1:1. T cell alone controls received 100 μL of either RPM1640 10% FBS. For coculture with SKMEL5, 50 μL of 1 E5/mL T cells were added to target cells plated the previous day (resulting in an E:T of 1:1), followed by addition of 50 μL of 0 or 20 ng/mL TGFβL. For both T2 and SKMEL5 cocultures, the final volume in the well was 200 μL, resulting in a TGFβ1 concentration of 0 or 5 ng/mL. Six replicates were set up for each condition and pooled at the end of the coculture into 3 replicates to ensure that wells contained enough T cells for robust evaluation of T cell proliferation. Plates were covered with BreathEasy membrane to minimize evaporation and were incubated at 37° C., 5% CO₂ for 3.5 days. At the end of the coculture, cell cocultures were stained with the reagent listed in Table 8. After staining, cells were fixed with Cytofix (BD Bioscience) prior to acquisition. Samples were acquired on a Cyoflex S (Beckman Coulter) using Cytexpert Software. Data were analyzed with Flowjo v10.8.1. Briefly, gates were set on TCRαβ⁺CD34⁺ (total transduced T cells), TCRαβ⁺CD34⁺CD4⁺CD8⁺ (transduced helper T cells) and CD34⁺ CD4⁺CD8⁺ T cells (transduced cytotoxic T cells). For TCR-T cells cocultured with SKMEL5, the percentage of dividing T cells was determined for the 3 subpopulations of transduced T cells (total, helper or cytotoxic T), and for TCR-T cells cocultured with T2 cells, the percentage of cells undergoing 4 or more cycles was determined. Subsequently, the percentage of proliferating cells observed in the 5 ng/mL TGFβ condition was normalized to the percentage of proliferating cells observed in the 0 ng/mL TGFβ condition and was plotted as heatmap in GraphPad Prism (v9.5.01).

TCR-T cells were labeled with CTV dye and were cocultured for 3.5 days with the HLA-A*02:01 positive and PRAME positive cell line SKMEL5 in the presence or absence of 5 ng/mL TGFβ. Subsequently, proliferation of transduced TCR-T cells was evaluated by flow cytometry using a gating strategy similar to the gating strategy described above. The data in FIG. 15B presents the percentage of cell proliferation observed in the presence of TGFβ normalized to the 0 ng/mL TGFβ condition; here, a value of 100% indicates that TGFβ3 did not inhibit proliferation, whereas any decrease induced by TGFβ is represented by a value smaller than 100%.

TGFβ suppressed the proliferation of both helper T cells and cytotoxic T cells of process-similar TCR-T cells lacking DN-TGFγRII (RG2959-164 and 6466-164 highlighted with asterisks in FIG. 15B). In contrast. TGFβ only marginally reduced the proliferation of cytotoxic T cells of TSC-203-A0201 batches expressing DN-TGFβRII (i.e., process-representative TCR-T cell batches PD314, PD315 and PD317 and process-similar TCR-T cell batches RG2959-134 and 6466-134). Indeed, while the percentage of proliferating cytotoxic T cells was reduced by 56% (RG2959-164) and 24% (6466-164) in the presence of TGFβrelative to the absence of TGFβ for TCR-T cells lacking DN-TGFβRII, proliferation of cytotoxic T cells expressing DN-TGFγRII only declined by 2% to 15%. Furthermore, helper T cells expressing DN-TGFβRII also appeared to resist TGFβ-mediated inhibition of proliferation when compared to helper T cell lacking DN-TGFβRII, although the protective effect of DN-TGFγRII was lower than what was measured with cytotoxic T cells; the percentage of proliferating helper T cells was reduced by 43% (RG2959-164) and 35% (6466-164) for helper TCR-T cells lacking DN-TGFβRII when exposed to TGFβ, while the reduction in proliferation ranged from 0% to 27% for helper TCR-T cells expressing DN-TGFβRII.

Overall, these data demonstrate that process-representative TSC-203-A0201 TCR-T cells maintain target-induced reactivity in the presence of physiological levels of TGFβ. DN-TGFγRII shields TSC-203-A0201 TCR-T cells from TGFβ-mediated suppression of IFN-γ secretion, as well as TGFβ-mediated inhibition of proliferation.

### Example 4: In Vivo Efficacy of TSC-203-A0201

In vivo anti-tumor activity, tolerability, and persistence of a representative number of independent donor-derived batches of TSC-203-A0201 TCR T cells prepared as described in Example 3 above and administered intravenously in a representative cancer model (xenograft model of Hs 695T in female NCG mice) were evaluated. TSC-203-A0201 TCR-T cells displayed a potent anti-tumor activity with all three batches. Based on body weight and clinical observations, TSC-203-A0201 treatments were well tolerated. No overt signs of toxicity were observed and there were no apparent treatment-related deaths.

In particular, efficacy of the 3 batches of TSC-203-A0201 from different donors was compared to non-engineered (untransfected [UTF]) control T cells from matching donors, and vehicle (PBS) treatments against the Hs 695T tumor cell line implanted in NCG female mice. Hs 695T cell line is derived from an amelanotic melanoma. These tumor cells endogenously express PRAME and HLA-A*02:01 and were confirmed to be recognized by TSC-203-A0201 TCR-T cells in vitro (see Example 3 above). Tumor cells were inoculated subcutaneously in the right flank of the animals. Once tumor engraftment was successful (tumors reaching 100 mm³ on average), animals were randomized into different treatment groups, and received two doses, 7 days apart, of TSC-203-A0201, of UTF control T cells, or of vehicle. The treatments were performed via intravenous (i.v.) injections. Different readouts were gathered over time: (1) anti-tumor efficacy was evaluated by biweekly tumor volume measurements; (2) biweekly body weight measurements were recorded alongside any clinical observations to gauge any toxicity related to TSC-203-A0201 injection.

Materials and methods used for in vivo efficacy tests are described herein. Briefly, the Hs 695T cell line, derived from the lymph node metastasis of an amelanotic melanoma was purchased from ATCC (catalog #HTB-137) and cultured according to the manufacturer's recommendations. The cell culture medium used to grow this cell line was EMEM (ATCC) supplemented with 10% FBS (Gibco). The tumor cells were maintained at log phase growth in tissue culture flasks in a humidified incubator at 37° C., in an atmosphere of 5% CO₂ and 95% air.

One hundred (100) female CR NOD-Prkdc^{em26Cd52}Il2r^{gem26Cd22}/NjuCrl (NCG) mice were ordered and inoculated with tumor cells for potential assignment to the study. The mice were 7 weeks old, with body weights ranging from 19.4 to 26.41 g at the beginning of the study.

Hs 695T cells used for subcutaneous xenografts were harvested during log phase growth and washed in PBS. Mice were inoculated s.c. with 100 μL of a mix of 50% Matrigel/50% medium containing 1 E6 live Hs 695T cells. Tumors were measured by caliper biweekly starting one week post inoculation and continued until the end of the study. Tumor measurements were recorded in the software Overwatch. When average tumor volumes reached 100 mm³ (6 days post inoculation, tumor volumes ranged from 85.03 mm³ to 118.52 mm³), animals were randomized to produce 7 groups of 12 mice for the study of antitumor efficacy. The day of animal randomization corresponds to Day 0 of the study.

On the days of dosing, cryo-bags (750 mL CryoMACS) were thawed using 37° C. water bath and transferred in thawing media consisting of X-VIVO 15 media (Lonza Cat #04-418Q), and 5% heat-inactivated human serum (Sigma Cat No. H3667), resuspended and rinsed in sterile PBS to wash the cells and resuspended in PBS to obtain dosing suspension at concentrations of 2 E7 viable CD34⁺ cells per 0.1 mL. The total amount of live T cell injected was adjusted to account for CD34⁺ purity. The number of UTF control T cells injected was adjusted to match the total number of live cells needed for TSC-203-A0201 of the matching batch.

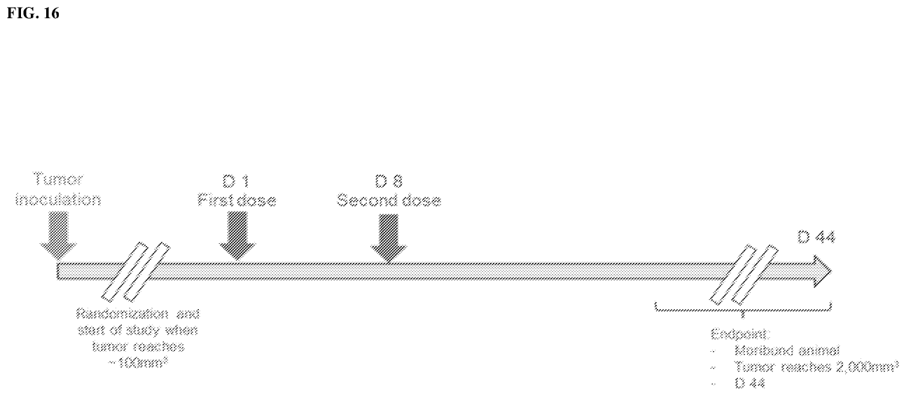

Animals received repeat doses of TSC-203-A0201 on Day 1, and Day 8 of the study according to the study design presented in Table 9 and FIG. 16. The dosing volume for all the test articles was 100 μL except for UTF PD314 (group 2) that received 200 μL to prevent cell clumping due to low cell viability. Group 1 received injections of vehicle control (PBS); group 3 received injections of TSC-203-A0201 from batch PD314 (2.0 E7 live CD34⁺ cells corresponding to 2.616 E7 total T cells); group 5 received injections of TSC-203-A201 from batch PD315 (2.0 E7 live CD34⁺ cells corresponding to 2.504 E7 total T cells); and group 7 received injections of TSC-203-A0201 from batch P1317 (2 E7 live C134+ cells corresponding to 2.512 E7 total T cells). The number of UTF control T cells injected for each group was matched with the total T cells injected for the respective TSC-203-A0201-treatment groups: group 2, group 4, and group 6 received live untransfected (UTF) control T cells from batch PD314 (2.616 E7 live T cells), PD315 (2.504 E7 live T cells) and PD317 (2.512 E7 live T cells), respectively.

TABLE 9
Experimental design and reagents
Experimental design

| Gr. | N | Agent | Formulation dose (CD34⁺) | Route | Schedule |
|---|---|---|---|---|---|
| 1 | 12 | Vehicle (PBS) | NA | i.v. | Days 1, 8 |
| 2 | 12 | PD314 - UTF | NA | i.v. | Days 1, 8 |
| | | | (total number of T cells | | |
| | | | matching group 3) | | |
| 3 | 12 | PD314 - TSC-203-A0201 | 2E7 cells/animal | i.v. | Days 1, 8 |
| 4 | 12 | PD315 - UTF | NA | i.v. | Days 1, 8 |
| | | | (total number of T cells | | |
| | | | matching group 5) | | |
| 5 | 12 | PD315 - TSC-203-A0201 | 2E7 cells/animal | i.v. | Days 1, 8 |
| 6 | 12 | PD317 - UTF | NA | i.v. | Days 1, 8 |
| | | | (total number of T cells | | |
| | | | matching group 7) | | |
| 7 | 12 | PD317 - TSC-203-A0201 | 2E7 cells/animal | i.v. | Days 1, 8 |

Reagents

| | Vendor | Catalog Number |
|---|---|---|
| Media | | |
| EMEM | ATCC | 30-2003 |
| Fetal Bovine Serum (Heat-Inactivated) | Gibco | A38400 |
| DPBS (Dulbecco's phosphate-buffered | Gibco | 14190-136 |
| saline) | | |
| Reagent | | |
| 0.4% Quatricide | Medline | N/A (From Explora) |
| Wescodyne | West Penetone | 1511 |
| DPBS | Gibco | 14190-136 |
| FBS | Gibco | 35050-001 |
| DMSO | Sigma | D265-100ml |
| GlutaMax (100x) | Gibco | 35050-061 |
| EMEM | ATCC | 30-2003 |
| ViaStain AOPI Staining Solution | Nexcelom Bioscience | CS2-0106-25mL |
| 70% Isopropanol | Decon Laboratories Inc | 8601 |

Throughout the study, mice were observed for general health/mortality and moribundity twice daily, once in the morning and once in the afternoon. Cage-side observations occurred daily. Mice were not removed from their cage during observation, unless necessary for identification or confirmation of possible findings. The mice were also observed for overt signs of any adverse treatment-related (TR) side effects, during sampling, body weight and tumor measurement activities.

Body weights were recorded on Days −3, 0, 4, 8, 11, 15, 18, 21, 23, 26, 30, 33, 37, and 40 of the study.

Tumors were measured by caliper (mm units) biweekly until 46 days post tumor inoculation and recorder in the software Overwatch. The greatest longitudinal diameter (length [L]) and the greatest transverse diameter (width [W]) were determined and reported into the Overwatch software. The tumor volume was estimated using the ellipsoidal formula V=(W²×L)/2.

The differences in the tumor growth over time between pairs of treatment groups were assessed by fitting each animal's data to a simple exponential growth model and comparing the mean growth rates of two groups. The difference in the growth rates was summarized by the Growth Rate Inhibition (GRI), which is the reduction in the growth rate experienced by the treatment group relative to that of the Control-treated group, expressed as a fraction of the vehicle growth rate. A positive GRI indicates that the tumors in the treatment group grew at a reduced rate relative to the reference group. GRI was calculated on Day 33 of a study, when all groups displayed 90-100% of groups animal alive. The tumor volumes were log-transformed, and the growth rate for each animal was calculated as the slope of the log volume vs. time.

$$GRI = 100 \% \times ( mean growth rate for control - mean growth rate for treatment ) \text{} / mean growth rate for control$$

The endpoint of the experiment was death or moribundity due to tumor progression, tumor volume reaching 2,000 mm³, or the last Day of the study (Day 40 on study, corresponding to Day 46 post-inoculation of tumor cells).

Animals were monitored individually for signs of moribundity, decreases in body weight and tumor progression and classified as death on study. The time to endpoint (TTE), in days, was recorded for each mouse that died of its disease or was euthanized due to tumor progression. Any animal classified as having died from treatment-related (TR) causes was assigned a TTE value equal to the day of death. Any animal that did not appear moribund but was euthanized due to disease progression as supported by necropsy observations, was recorded as a non-treatment-related death due to tumor invasion or metastasis (NTRm) and was included in the data analysis. Any death due to unknown causes (NTRu) or due to an accident or error (NTRa) was excluded from TTE calculations and all further analyses.

Mice were euthanized by asphyxiation using Carbone Dioxide (CO₂) followed by cervical dislocation. The time to endpoint (TTE), in days, is recorded for each mouse that dies of its disease or was euthanized due to tumor progression or study termination.

Pairwise comparisons of growth rate inhibition (GRI) were performed, where the mean GRI for the vehicle control treated animals was compared with GRI of different treatment conditions. The growth rate estimates were assumed to be normally distributed, and an unpaired t-test with unequal variances was used to check if there was a statistically significant difference between the two groups. Statistical analyses were performed using Student's t-test in GraphPad Prism® 8.0 software and P-values <0.05 were considered statistically significant. All days and all animals were included.

a. TSC-203-A0201 TCR-T Cells Exhibit In Vivo Anti-Tumor Efficacy

The efficacy of TSC-203-A0201 TCR-T cells was evaluated against the Hs 695T tumor model in NCG female mice. Three batches of process-representative TSC-203-A0201 material were compared to untransfected (UTF) control T cells from matching donors, and to vehicle control (PBS) treatment. Animals with confirmed growing tumors (with average tumor volumes of about 100 mm³; tumor volumes ranged from 85.03 mm³ to 118.52 mm³; 6 days post inoculation) were randomized (Day 0 of the study) into different experimental groups as shown in Table 9 and received two doses of treatment regimen from one of the 3 batches of process-representative TSC-203-A0201 material, or respective UTF control T cells, or vehicle (PBS). The TSC-203-A0201 treatment groups were injected with 2 E7 live CD34⁺ (i.e., engineered) TCR-T cells on study Day 1 and Day 8. Cellular dose was adjusted for the purity of the material and corresponded to 2.616 E7 total T cells for batch PD314, 2.504 E7 total T cells for batch PD315 and 2.512 E7 total T cells for batch PD317, respectively. The number of UTF control T cells injected for each UTF control group matched the total T cells injected for the respective TSC-203-A0201-treatment groups.

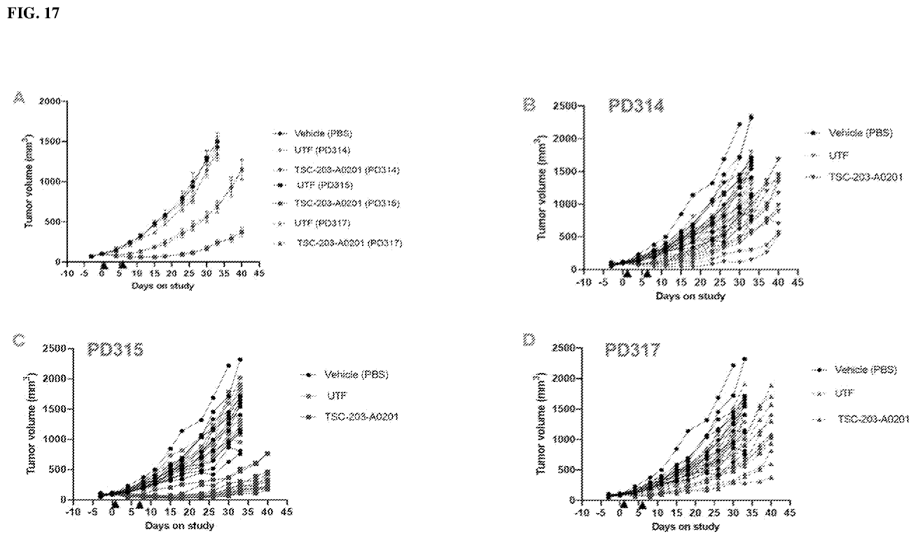

As presented in FIG. 17, animals for control groups (groups 1 [vehicle], 2, 4 and 6 [UTF control T cells from batches PD314, PD315 and PD317, respectively]) presented large tumors at the end of the study, with mean tumor volumes on Day 33 of 1433.16 mm³, 1427.11 mm³, 1500.03 mm³ and 1348.65 mm³, respectively. The median TTE was 33 days for all these control groups. Tumors in UTF-treated control groups (groups 2, 4 and 6) grew at a similar rate as the vehicle-treated group (group 1), with 0.42%. −4.66% and 5.89% tumor growth inhibition for groups 2, 4 and 6, respectively (calculated on Day 33 of study). There were no statistical differences when considering GRI of groups 2, 4 or 6, over the tumor growth of vehicle group 1 (P-values of 0.8453, 0.9860 and 0.6906 when comparing GRI for group 2, 4 and 6, respectively).

The groups that received injections of TSC-203-A0201 TCR-T cells (groups 3, 5 and 7, testing TCR-T cells from batch PD314, 315 and PD317, respectively) showed anti-tumor response when compared to the matching UTF control T cells-treated groups (groups 2, 4 and 6). The median TTE for the treatment groups was 40 days (corresponding to the end of the study) which was significantly longer than the TTE observed for the vehicle control group (P-values <0.0001). In addition, both TSC-203-A0201-treated groups presented a significant tumor GRI of 51.82% (group 3), 83.92% (group 5), and 49.59% (group 7) relative to their respective the control-vehicle group (P-values of 0.0526, 0.0032, and 0.0615 for group 3, 5, and 7, respectively).

b. TSC-203-A0201 TCR-T Cells Exhibit No In Vivo Toxicity

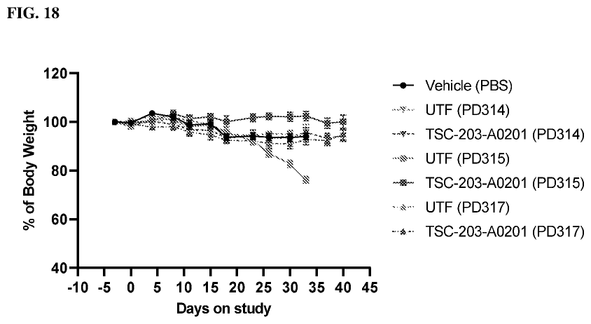

No apparent treatment-related deaths were observed. All study groups showed a comparable trend of BW as the vehicle between Day 1 up to Day 40 of study (FIG. 18) apart from animals of group 4 (receiving UTF control T cells from batch PD315). Specifically, the body weight of TSC-203-A0201-treated animals appeared similar to that of vehicle treated animals, suggesting no overt signs of toxicity.

### Example 5: Identification of Putative Off-Targets for TSC-203-A0201

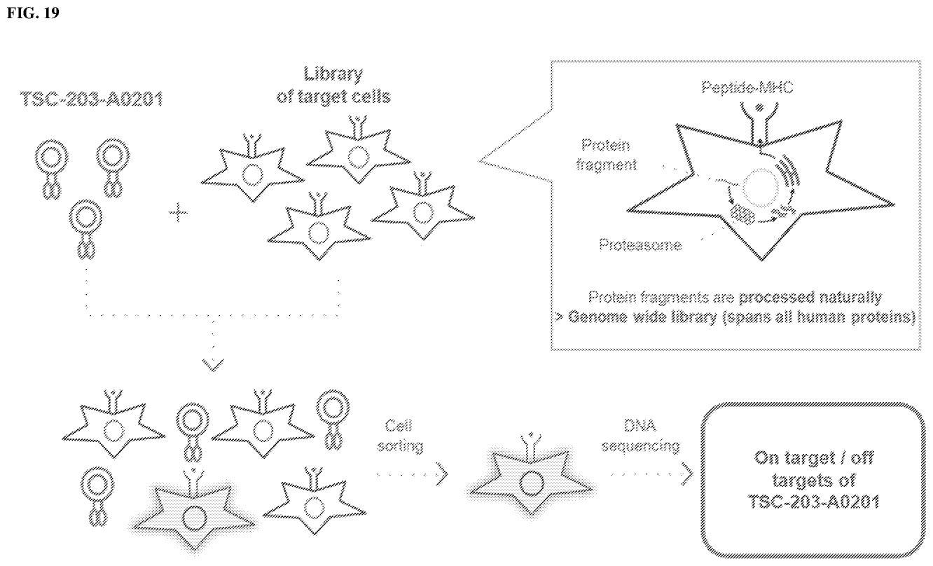

Any potential off-targets of the therapeutic TCR used in TSC-203-A0201 using TScan's proprietary genome-wide screen called Target Scan. Off-target epitopes recognized by a TCR have traditionally been predicted using computational algorithms or screening positional scanning libraries. TScan takes an unbiased and comprehensive approach by using a proprietary genome-wide safety screening platform: the Target Scan screen (Kula et al. (2019) Cell 178:1016-1028; Ferretti et al. (2020) Immunity 53:1095-1107). This approach enables identification of epitopes recognized by a given TCR even if they have low sequence homology with the on-target epitope and are therefore unlikely to be predicted using computational methods or a more focused mutational screen (FIG. 19).

The Target Scan screen is a high-throughput cell-based technology that enables comprehensive identification of the natural targets and potential off-targets of CD8⁺ T cells in an unbiased, genome-wide fashion. Briefly, CD8+ T cells expressing the TCR of interest (in this case, the recombinant TCR for TSC-203-A0201; hereafter referred to as mechanistically representative TSC-203-A0201 TCR-T cells) are cocultured with HEK293T target cells that express a genome-wide library of protein fragments and have been engineered to express the HLA of interest (in this case HLA-A*02:01). Target cells also express a reporter that becomes fluorescent in the presence of granzyme B activity. Each target cell in the library also expresses a different ˜90-amino acid protein fragment (also known as a 90mer). Collectively, the library expresses fragments that span every human protein in the proteome and include all common single nucleotide polymorphisms. These fragments are processed naturally by the target cells, and peptides derived from the fragments are displayed on class I MHCs (in this case HLA-A*02:01) on the cell surface. If an engineered CD8⁺ T cell encounters a peptide/MHC target that it recognizes in the coculture, the T cell secretes cytotoxic granules into the target cell, triggering the target cell to become fluorescent. Early apoptotic cells are isolated from the co-culture and fluorescent cells are further purified by fluorescence-activated cell sorting. The expression cassettes of the sorted cells are then sequenced, revealing the identities of the protein fragments expressed in those cells. The protein fragment encoding an epitope recognized by the TCR would exhibit a fold increase in the sorted cells compared to library input.

The Target Scan screen is enabled by two proprietary components: (i) TScan's granzyme B-activated infrared fluorescent protein (IFP) reporter allows cells targeted by a specific TCR to be identified, sorted, and analyzed; and (ii) TScan's target-cell libraries encompass a broad range of protein fragments that provide comprehensive, proteome-wide evaluation of all potential targets and off-targets of a given TCR. The primary screen used to analyze the therapeutic TCR of TSC-203-A0201 was performed using a proteome-wide library comprising ˜600.000 clones. As part of the Target Scan screening process, TScan routinely analyzes the starting library by next generation sequencing. This effort verified that >97% of the clones in the library are represented in the screens. On average, each clone is typically present at >10,000 copies in the coculture.

Besides confirming that the therapeutic TCR used in TSC-203-A0201 recognizes its intended target (i.e., the PRAME epitope), the data presented herein demonstrate that the proteome-wide screen identified PLA2G4E, SLC26A1, and EFNA1 as putative off-targets for the recombinant TCR. All are expressed in various tissues and could present a risk of off-tumor activity for TSC-203-A0201 in patients. Follow up experiments using functional assays were performed to assess the actual reactivity of process representative TSC-203-A0201 TCR-T cells against these putative off-targets to determine the risk (or lack thereof) of off-tumor reactivity. These follow up functional data are presented in Example 6 below. These follow-up efforts confirmed that TSC-203-A0201 TCR-T cells lack reactivity to cells endogenously expressing the putative off-target proteins identified here, indicating that none of these putative off-targets pose a risk of off-tumor reactivity for TSC-203-A0201. Instead, the TCR-T cells present a selective reactivity to the PRAME-derived epitope presented on HLA-A*02:01.

First, the lentivirus was packaged and the viral titer was determined. Lenti-X cells (Takara Bio USA, Mountain View, CA) were plated at 75% confluency and transfected using jetPRIME transfection reagent (Polyplus. Illkirch, France). Briefly, lentiviral constructs were mixed with packaging plasmids (pREV/pTAT/pVSVG/pGAGPOL) and incubated with jetPRIME reagent according to the manufacturer's protocol, and Opti-Pro SFM medium was added at 24 hours post-transfection. Viral supernatants were harvested 48 hours after transfection. Viral supernatants to produce mechanistically representative TSC-200-A0201 TCR-T cells were concentrated using either Vivaspin 20 centrifugal concentrators or Vivaflow 50 cassettes (Sartorius, Bohemia, NY). Virus titer for supernatants was determined by RT-qPCR using Luna® Universal One-Step RT-qPCR Kit (New England BioLabs) on a QuantStudio 7 Pro (ThermoFisher Scientific). Briefly, virus was diluted in PBS, lysed and 2 μl of the lysed virus was used in RT-qPCR according to manufacturer's instructions. Standard curve was generated using serial dilutions of virus with known titer. Titer was expressed as transduction units (TU)/ml. For peptide library virus, lentiviral titer was determined by puromycin colonies formation using Lenti-X™ cells transduced with serial dilution of viral supernatant. To quantify viral titers, puromycin resistance colonies were selected 48 hours post-transduction. Puromycin resistance colonies were visualized by crystal violet staining and counted. Titer was calculated as the colony forming unit as TU/ml using the formula: TU/ml=Number of puromycin resistance colonies×dilution factor×1000.

Reporter cells were prepared. Briefly, reporter cells were generated by knocking endogenous HLA-A/B/C out in HEK293T cells using CRISPR and engineered to express a granzyme-activated infrared fluorescent protein (IFP) (Kula et al. (2019) Cell 178:1016-1028) and a granzyme-activated scramblase (Ferretti et al. (2020) Immunity 53:1095-1107). Reporter cells for screening mechanistically representative TSC-203-A0201 TCR-T cells were engineered to express HLA-A*02:01. Reporter cells endogenously PRAME (79 TPM as determined by inhouse RNAseq analysis). To prevent PRAME-mediated killing of reporter cells by TSC-203-A0201 TCR-T cells, PRAME was knocked out of reporter cells using CRISPR. Each PRAME-targeting guide RNA was assembled by complexing with TracrRNA with individual crRNAs. Single guide crRNAs (Integrated DNA Technologies) and TracrRNA (Integrated DNA Technologies) were resuspended at 100 μM in IDTE buffer. Individual crRNAs were mixed with TracrRNA at 1:1 v/v ratio and heated to 95° C. for 5 minutes in a heat block or thermocycler. The annealed RNA was allowed to cool to room temperature. Ribonucleoprotein complexes (RNPs) were assembled by incubating the individual annealed RNA duplexes with Cas9 (IDT DNA Technologies) at room temperature for 15 minutes. Assembled RNP was frozen and stored at −20° C. for later use.

Reporter cells expressing HLA-A*02:01 were electroporated using the Amaxa SF Cell Line 4D-Nucleofector Kit (Lonza), 3 E5 reporter cells were electroporated with two PRAME-targeting RNPs and 25 μM Electroporation Enhancer (Integrated DNA Technologies) using program CM-130 on the Lonza 4D-Nucleofector System. Following electroporation, the cells were expanded and then sequentially co-cultured with two A*02:01-restricted PRAME-specific TCR-T cells to allow for the selection of reporter cells with reduced expression of PRAME. The TCR-T cells used to select for the PRAME-negative reporter cells are lost during the expansion and successive passages of the reporter cells.

The human genome-wide peptide library was generated by tiling across the human genome coding sequences spanning all proteins of the human genome with overlapping 90-mer amino acid tiles. The tiles were synthesized on a silicon chip (Twist Bioscience) and cloned into a lentivirus expression vector.

Reporter cells were transduced with lentiviral particles to express the PEP LIB V2 PLUS library at an MOI of 5 in complete DMEM (1×DMEM supplemented with 10% fetal bovine serum, 100 IU/mL penicillin, 100 μg/mL streptomycin). This library is comprised of >600,000 constructs (tiles) comprised of 90 amino acids each, representative of the human proteome. The day after transduction, complete DMEM media was refreshed, and cells were maintained in culture or frozen in complete DMEM supplemented with 10% DMSO until needed.

Mechanistically representative TSC-203-A0201 TCR-T cells were generated. Briefly, Primary CD8⁺ T cells from a single donor were isolated using the StraightFrom Leukopak CD8 Microbead Kit (Miltenyi Biotec) according to the manufacturer's protocol. Isolated cells were frozen in CryoStor® CS10 (Stem Cell Technologies) and stored in liquid nitrogen until use. On Day −1, CD8+ T cells were thawed, washed with complete T cell medium (RPMI 1640 supplemented with 10% heat-inactivated fetal bovine serum [FBS], 100 IU/mL penicillin, 100 μg/mL streptomycin, recombinant human IL-2 [50 U/mL, PeproTech, Cranbury, NJ], recombinant human IL-15 [5 ng/mL, R & D Systems], and recombinant human IL-7 [5 ng/mL, R & D Systems]), seeded at 1.0 E6/mL, and rested overnight at 37° C. On Day 0, CD8⁺ T cells were washed and resuspended in fresh T cell medium and activated using ImmunoCult™ human CD3/CD28/CD2 T cell activator (5 mL/1.0 E6 CD8⁺ T cells, Stem Cell Technologies). On Day 1, cells were washed and resuspended in fresh complete T cell medium and transduced with lentiviral particles to express the recombinant TCR for TSC-203-A0201.

On Day 2, cells were washed and resuspended in fresh complete T cell medium and expanded until Day 5 in G-Rex® 6-well plates (Wilson Wolf). On Day 5, cells were isolated using fluorescent activated cell sorting (FACS). Cells were harvested and resuspended in EasySep™ buffer (StemCell Technologies) and incubated with HLA-A*02:01/SLLQHLIGL Dextramer-APC (Immudex, 1:25-1:50 dilution) at 4° C. for 20 minutes. Anti-CD34-Alexa Fluor®488 (R & D Systems, 1:50 dilution) was added directly to cells and incubated at 4° C. for an additional 10-20 minutes. Cells were washed with EasySep™ buffer, centrifuged, and resuspended in EasySep™ buffer containing DAPI (1:200 dilution). Live dextramer binding (DAPI− CD34⁺ Dextramer⁺) T cells were sorted on a MoFlo Astrios EQ cell sorter (Beckman Coulter).

The isolated cells were resuspended in fresh complete T cell medium and expanded in G-Rex® 10 flasks (Wilson Wolf) until Day 12, at which point the cells were frozen down in CryoStor® CS10 and stored in liquid nitrogen until used.

Mechanistically representative TSC-203-A0201 TCR-T cells were thawed and restimulated (further expanded) by coculturing T cells with irradiated (60 grays) allogeneic PBMCs in the presence of 0.1 μg/mL anti-CD3 (OKT3, eBioscience) and 50 U/mL recombinant IL-2 (Peprotech) in fresh T cell medium (RPMI 1640 supplemented with 10% heat-inactivated fetal bovine serum [FBS], 100 IU/mL penicillin, 100 μg/mL streptomycin, recombinant human IL-2 [50 U/mL, PeproTech]) in G-Rex® 100 flasks (Wilson Wolf), 50 U/mL recombinant IL-2 was added to expanding cells every other day until Day 6. On Day 6, half of culture medium was replaced with fresh T cell medium containing 50 U/mL recombinant IL-2. Cells were used for screens on Day 7.

Target Scan screen was then carried out. Specifically, on Day 5 of restimulation of mechanistically representative TSC-203-A0201 TCR-T cells, reporter cells (expressing the peptidome library) were labeled with Cell Trace Violet (Thermo Fisher) for 10 minutes at room temperature. Labeling reaction was quenched with a 5× excess of complete DMEM media (1×DMEM supplemented with 10% FBS, 100 IU/mL penicillin, 100 mg/mL streptomycin). After centrifugation, 4 E8 labelled reporter cells were seeded in a CellSTACK flask.

On Day 6 of restimulation. T cells' reactivity against the PRAME epitope and the ability of the reporter cells to respond to granzyme B signaling were confirmed prior to engaging in the screen coculture. A fraction of reporter cells was pulsed with 100 ng/mL of PRAME peptide (SLLQHLIGL. Genscript) for one hour. T cells were added to reporter cells at four effector to target (E:T) ratios (2:1, 1:1, 1:2, 1:4) in triplicate. After four hours of incubation at 37° C., cells were resuspended by pipetting up and down before data acquisition on a CytoFLEX flow cytometer (Beckman Coulter).

On Day 7 of restimulation, mechanistically representative TSC-203-A0201 TCR-T cells were added to library-transduced reporter cells and incubated at 37° C. for four hours. After incubation, all cells were harvested by trypsinization and centrifugation, resuspended in 1× Annexin V binding buffer (Miltenyi Biotec), and centrifuged. Cells were resuspended with Annexin V magnetic microbeads (Miltenyi Biotec) in 1× Annexin V binding buffer (1 mL microbeads in 9 mL Annexin V binding buffer per 1 E9 total cells) and incubated at room temperature for 15 minutes. Cells were washed with 5× volume of Annexin V binding buffer and centrifuged. Cells were resuspended in Annexin V binding buffer and then divided to 2 replicates and filtered using a 70 μM cell strainer (Corning). Annexin V-labeled cells were positively selected using an AutoMACS Pro (Miltenyi Biotec). The elution of each replicate was further divided over four sort replicates for a total of 8 technical replicates per screen. IFP⁺ cells were sorted using a MoFlo Astrios EQ cell sorter (Beckman Coulter). The E:T ratio for the mechanistically representative TSC-203-A0201 TCR-T cells in this off-target screen was 1:1.5.

Genomic DNA (gDNA) was extracted from sorted cells using the GeneJET Genomic DNA Purification Kit (Thermo Fisher Scientific). The antigen cassette was amplified from the extracted gDNA by PCR, then appended with sequencing adaptors, and sample-specific index sequences in a second PCR reaction. Amplicons were sequenced on an Illumina® NextSeq using the standard llumina sequencing primer.

Nucleotide sequences were mapped to individual nucleotide tiles. The proportion of read counts for each tile was calculated for each screen replicate (n=8) and for the input for each pool of transduced reporter cells. Enrichments of each tile were calculated by dividing the proportion of the tile in the screen replicate by the proportion of the tile in the input library. A modified geometric mean of the enrichment of an identical tile across the 8 screen replicates was used to identify reproducible screen hits. Any sequence which showed an enrichment over background across multiple 90-mer tiles was considered a ‘hit’.

TABLE 10
Cell Lines

| Cell Line | Vendor | Catalog Number |
|---|---|---|
| HEK293T | ATCC | CRL-3216 |
| Lenti-X | Takara Bio | 632180 |
| Jurkat | ATCC | TIB-152 |

Media and Supplements

| Media | Vendor | Catalog Number |
|---|---|---|
| Penicillin streptomycin | Gibco | 15149-122 |
| RPMI-1640 Medium | ATCC | 30-2001 |
| Fetal Bovine Serum (FBS), heat inactivated | Gibco | A38401012 |
| DMEM (1x) [+] 4.5 g/L D-glucose, [+] | Gibco | 11965 |
| L-glutamine, [−] Sodium Pyruvate | | |
| OptiPRO SFM | Gibco | 12309019 |
| 1x DPBS (no Ca2+/Mg2+) | Gibco | A3840102 |
| Puromycin Dihydrochloride | Gibco | A1113803 |
| MACS Running buffer | Miltenyi | 130-091-221 |
| EasySep ™ Buffer | StemCell Technologies | 20144 |
| CryoStor ® CS10 | StemCell Technologies | 07930 |
| Lymphocyte Separation Medium | Corning | 25-072-CV |

Cytokines

| Reagent | Vendor | Catalog Number |
|---|---|---|
| Recombinant human IL-2 | Peprotech | 200-02 |
| Recombinant human IL-7 | R&D Systems | 207-11-005 |
| Recombinant IL-15 | R&D Systems | 247-ILB-005 |

Kits

| Reagent | Vendor | Catalog Number |
|---|---|---|
| Straight From Leukopak CD8 Microbead Kit | Miltenyi Biotech | 130-117-019 |
| Annexin V Microbead Kit | Miltenyi Biotech | 130-090-201 |
| SF Cell Line 4D-Nucleofector ™ X Kit S | Lonza | V4XC-2032 |

Antibodies and Staining Reagents

| Reagent | Vendor | Catalog Number |
|---|---|---|
| Anti-human HLA-A, B, C PE (clone: W6/32) | BioLegend | 311406 |
| Cell Trace Violet (CTV) | Fisher Scientific | C34571 |
| Anti-human CD3 (clone: OKT3) | Invitrogen | 16-0037-85 |
| Anti-human CD34-Alexa Fluor 488 (clone: Qbend10) | R&D Systems | 310916 |
| ImmunoCult ™ Human CD3/CD28/CD2 T cell | StemCell Technologies | 10970 |
| activator | | |
| DAPI (4′,6-diamidino-2-phenylindole) | Thermo Scientific | 62248 |
| 7-AAD Viability Staining Solution | BioLegend | 420404 |
| ViaStain AO/PI staining solution in PBS | Nexcelom Bioscience | CS2-0106-5ml |

Dextramers and Peptides

| Reagent | Vendor | Catalog Number |
|---|---|---|
| PRAME₄₂₅₋₄₃₃ peptide (SLLQHLIGL) | GenScript | custom |
| HLA-A*02:01/SLLQHLIGL Dextramer-APC | Immudex | custom |

Genomics Reagents

| Reagent | Vendor | Catalog Number |
|---|---|---|
| GeneJet Genomic DNA Purification kit | Thermo Fisher | K0722 |
| | Scientific | |
| QIAamp DNA Mini Kit | Qiagen | 51304 |
| Phusion HF PCR Master Mix Buffer | Thermo Fisher | F521L |
| | Scientific | |
| Monarch PCR & DNA Cleanup Kit | New England Biolabs | T1030L |
| Nuclease Free Water | Corning | 3524 |
| jetPRIME Transfection Reagent | Polyplus-transfection | 114-75 |
| Vivaspin 20 | Sartorius | VS2041 |
| Vivaflow 50 cassettes | Sartorius | VF05P4 |
| Luna ® Universal One-Step RT-qPCR Kit | New England | E3005X |
| | BioLabs | |
| Puromycin | Gibco | A11138-03 |
| Alt-R ® CRISPR-Cas9 crRNA, GCCGGCAGTTAGTTATTGAG | Integrated DNA | Custom |
| | Technologies | |
| Alt-R ® CRISPR-Cas9 crRNA, CTAACTGCCGGCTTTCGGTT | Integrated DNA | Custom |
| | Technologies | |
| Alt-R ® CRISPR-Cas9 tracrRNA | Integrated DNA | 1072533 |
| | Technologies | |
| Alt-R ® S.p. CRISPR-Cas9 Nuclease V3 | Integrated DNA | 1081059 |
| | Technologies | |
| Alt-R ® Cas9 Electroporation Enhancer | Integrated DNA | 10007805 |
| | Technologies | |
| IDTE (1X TE Solution) | Integrated DNA | 11-01-02-05 |
| | Technologies | |

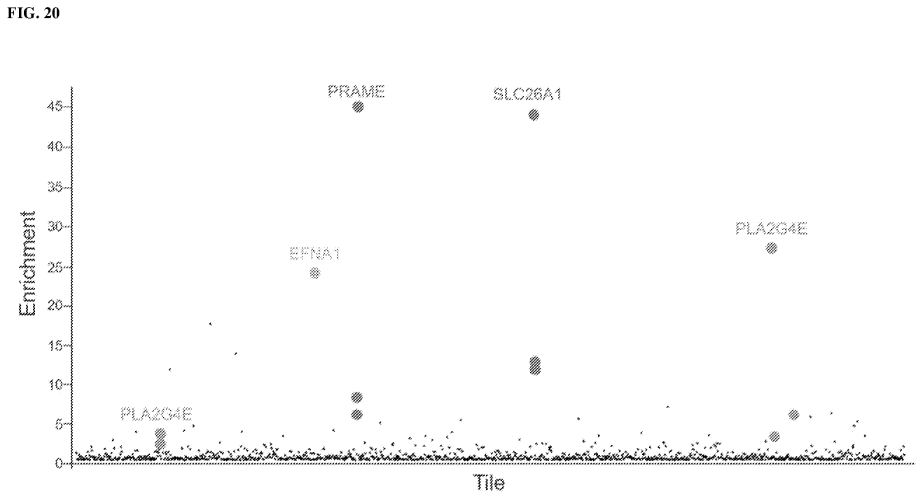

The results of the Target Scan screen are shown in FIG. 20 in the form of a dot plot where each of the ˜600,000 tiles forming the proteome-wide library are ordered along the X-axis, and the fold-enrichment for each tile, compared to the input library, is indicated on the Y axis. Out of the ˜600,000 clones expressing protein fragments that collectively spanned every human protein and covered all common SNPs, only 12 tiles were significantly enriched when the mechanistically representative TSC-203-A0201 TCR-T cells were evaluated. Besides the 3 tiles corresponding to the portion of PRAME producing the HLA-A*02:01-restricted epitope, the remainder of the enriched clones represented 3 potential off-targets for TSC-203-A0201. Overlapping protein tiles and/or concatemers derived from common SNPs were enriched from 2 genes: SLC26A1 and PLA2G4E. One tile derived from the N-terminus of EFNA1 was enriched. The N- and C-termini regions of proteins are underrepresented in the peptidome library, generally resulting in fewer overlapping tiles scoring in genome-wide screens.

The AA sequences of the enriched tiles are shown in Table 11 with the on-target PRAME epitope (SLLQHLIGL) bolded in white across the 3 scoring tiles. Also bolded are the overlapping AA across the tiles for a given putative off-target.

TABLE 11
Sequences of Scoring Tiles

| Tile ID | GENE | AA sequence |
|---|---|---|
| NM_006115-9-A | PRAME | LTDVSPEPLQALLERASATLQDLVFDECGITDDQLLALLP |
| | | SLSHCSQLTTLSFYGNSISISALQ SNLTHVLY |
| | | PVPLESYED |
| NM_006115-9.5 | PRAME | LVFDECGITDDQLLALLPSLSHCSQLTTLSFYGNSISISAL |
| | | Q SNLTHVLYPVPLESYEDIHGTLHLERLAYLH |
| | | ARLRELLC |
| NM_006115-10-B | PRAME | SQLTTLSFYGNSISISALQ SNLTHVLYPVPLE |
| | | SYEDIHGTLHLERLAYLHARLRELLCELGRPSMVWLSAN |
| | | PCPHCGDRTF |
| NM_022042-4.5 | SLC26A1 | NSSTLNGSAAMLDCGRDCYAIRVATALTLMTGLYQVLMG |
| | | VLRLGFVSAYLSQPLLDGFAMGASVTILTSQLKHLLGVRI |
| | | PRHQGPGMVVL |
| NM_022042-5-A | SLC26A1 | ATALTLMTGLYQVLMGVLRLGFVSAYLSQPLLDGFAMGA |
| | | SVTILTSQLKHLLGVRIPRHQGPGMVVLTWLSLLRGAGQ |
| | | ANVCDVVTSTVC |
| NM_022042-6-B | SLC26A1 | SQLKHLLGVRIPRHQGPGMVVLTWLSLLRGAGQANVCD |
| | | VVTSTVCLAVLLAAKELSDRYRHRLRVPLPTELLVIVVATL |
| | | VSHFGQLHKRF |
| NM_001080490-8-B | PLA2G4E | LQKRKVVVAKALKQVLQLEEDLQEDEVPLIAIMATGGGTR |
| | | SMTSMYGHLLGLQKLNLLDCASYITGLSGATWTMATLY |
| | | RDPDWSSKNLEP |
| NM_001080490-8.5 | PLA2G4E | QEDEVPLIAIMATGGGTRSMTSMYGHLLGLQKLNLLDCA |
| | | SYITGLSGATWTMATLYRDPDWSSKNLEPAIFEARRHVV |
| | | KDKLPSLFPDQL |
| REF3899_REF16952_ | ANKRD2_PLAZG4E_ | QEEENEKLRGDARQKLPMDLLVMYGHLLGLQKLNLLDC |
| REF16571_REF2278_ | SCARA3_VCX2 | ASYITGRGAPGPPGPRGFKGDMGVKGPVEGTQHDPLSQ |
| REF2443 | | ESEVEEPLSQESSE |
| REF4880_REF2089_ | UGT2B15_APOL1_ | LEVYPTSLTKNYLEDSLLKILDWFLKEFPRLKSELEDNIRR |
| REF16952_REF19825 | PLA2G4E_GP6 | LRAMYGHLLGLQKLNLLDCASYITGASLYSQQLRGLRW |
| | | ENGLSLGGRSE |
| SNP16952_SNP18557_ | PLAZG4E_ELSPBP1_ | MYGHLLGLQKLSLLDCASYITGSQGSFLGSLWWSVTSV |
| SNP23801_SNP18542 | AC135068.1_OR8H3 | FDEKQQDSYFCDITQVVQIACANTFPEEISFTGCFAQMFF |
| | | FVFLGTAECYSE |
| NM_004428-1-A | EFNA1 | MEFLWAPLLGLCCSLAAADRHTVFWNSSNPKFRNEDYTI |
| | | HVQLNDYVDIICPHYEDHSVADAAMEQYILYLVEHEEYQL |
| | | CQPQSKDQVRW |

Screening of the mechanistically representative TSC-203-A0201 TCR-T cells against PGP-32 DN the human proteomic revealed the putative off-targets of the therapeutic TCR: SLC26A1, PLA2G4E, and EFNA1. Consensus gene expression data from the Human Protein Atlas suggest that several of the genes are ubiquitously expressed with others showing tissue-specific expression (summarized in Table 12). The putative off-targets would represent significant off-tumor reactivity risk if confirmed to represent bona fide off-targets of TSC-203-A0201.

TABLE 12
Summary of tissue specificity and function of the putative
off-target proteins identified for TSC-203-A0201

| Gene | Protein | Tissue Specificity | Summary |
|---|---|---|---|
| SLC26A1 | Solute carrier family | Ubiquitious, but | SLC26A1 is an anion transporter |
| | 26 member 1 | predominantly in the liver | involved in the transport of sulfate, |
| | | | bicarbonate, and oxalate (Krick et al., |
| | | | 2009). |
| PLA2G4E | Phospholipase A2 | Predominantly in skin; | PLA2G4E is a calcium-dependent N- |
| | group IVE | weakly in esophagus, | acyltransferase (Murakami et al., 2020). |
| | | vagina, and testis | |
| EFNA1 | Ephrin A1 | Ubiquitous | EFNA1 is a cell membrane-bound |
| | | | ligand for the Eph receptor tyrosine |
| | | | kinases (Hao and Li, 2020). |

### Example 6: Lack of TSC-203-A0201TCR-T Cell Off-Tumor Reactivity

The risk of on-target/off-tumor reactivity is low for TSC-203-A0201 TCR-T cells as PRAME expression is largely restricted to the testis (Ikeda et al. (1997) Immunity 6:199-208; Xu et al. (2020) 53:p.e12770; Wadelin et al. (2010) Molecular cancer 9:1-10) which is an immune privileged tissue (Li et al. (2012) Front Immunol 11:152; Hedger (2015) Knobil and Neill's Physiology of Reproduction 2015: 805-892). Data from Example 8 below show that PRAME expression is largely restricted to the testes, and compared to testes lower levels of PRAME expression were detected in the kidney (˜50-fold lower than testes), ovary (˜25-fold lower than testes), and adrenal glands (˜6-fold lower than testes).

The off-target reactivity of a therapeutic TCR arises from the cross-reactivity of the TCR to a self-peptide/MHC.

As described below, TSC-203-A0201 TCR-T cells were determined not to react to a wide variety of primary samples including cells that endogenously express the putative off-target peptides such that reactivity of the TCR-T cells is expected to be restricted to cancer cells expressing PRAME and HLA-A*02:01.

Generally, TSC-203-A0201 TCR-T cells were tested for their reactivity to an extensive panel of 67 target cells comprising 7 cancer cell lines and 60 healthy human primary cells and iPSC-derived cells from multiple tissues and organs (Table 13).

TABLE 13
Target cancer cell line and primary cell descriptions
Target cancer cell line descriptions

| | | Catalog | Culture Medium |
|---|---|---|---|
| Cell Line | Vendor | number | (catalog number) |
| LS1034 | ATCC | CRL-2158 | RPMI-1640 (ATCC 30-2001) + |
| | | | 10% HIFBS + 1X Pen/Strep |
| NCI-H1666 | ATCC | CRL-5885 | RPMI-1640 (ATCC 30-2001) + |
| | | | 5% HIFBS + 1X Pen/Strep |
| BICR 56 | ECACC | 06031002-1VL | DMEM (Thermo Fisher 11965- |
| | (Sigma) | | 092) + 10% FBS + 1X P/S + |
| | | | 400 ng/mL hydrocortisone |
| CAMA-1 | ATCC | HTB-21 | EMEM (ATCC 30-2003) + 10% |
| | | | HIFBS + 1X Pen/Strep |
| 647-V | DSMZ | ACC 414 | 85% Dulbecco's MEM (Thermo |
| | | | Fisher 11965-092) + 15% h.i. |
| | | | FBS + 2 mM L-glutamine |
| NCI-H2228 | ATCC | CRL-5935 | RPMI-1640 (ATCC 30-2001) + |
| | | | 10% HIFBS + 1X Pen/Strep |
| SK-MEL-5 | ATCC | HTB-70 | EMEM (ATCC 30-2003) + 10% |
| | | | HIFBS + 1X Pen/Strep |
| CaSki | ATCC | CRM-CRL- | RPMI-1640 (ATCC 30-2001) + |
| | | 1550 | 10% HIFBS + 1X Pen/Strep |

Target primary and iPSC-derived cell description

| Primary/IPS- | Lot/batch | Catalog | | Culture Medium |
|---|---|---|---|---|
| derived cell type | number | number | Vendor | (catalog number) |
| Peripheral Blood | 12429 | | AllCells | X-VIVO15 (Lonza 04-418Q) + |
| Mononuclear Cells | 888607014 | | DLS | 5% Human serum (Sigma- |
| (PBMC) | 110044327 | | DLS | Aldrich H3667-100) + 1X |
| | | | | GlutaMax (FisherScientific |
| | | | | #35050061) + 1X |
| | | | | Penicillin/Streptomycin |
| | | | | (Thermofisher Scientific |
| | | | | 15149-122) |
| Human Umbilical | 474Z035 | A-12991 | PromoCell | Endothelial Cell Growth |
| Vein Endothelial | 474Z036 | C-12206 | | Medium 2 (C-22011) |
| Cells (HUVEC) | | | | |
| Normal Human | 471Z018.2 | C-12300 | PromoCell | Fibroblast Growth Medium (C- |
| Dermal Fibroblasts | | | | 23010) |
| (NHDF) | | | | |
| Human Pulmonary | 1081503.2 | C-12360 | PromoCell | Fibroblast Growth Medium 2 |
| Fibroblasts (HPF) | 474Z031.2 | | | (C-23020) |
| | 474Z024.2 | | | |
| Human Cardiac | 475Z017.1 | C-12375 | PromoCell | Fibroblast Growth Medium 3 |
| Fibroblasts (HCF) | 472Z002.2 | C-12375 | | (C-23025) |
| | 470Z011.10 | A-12992 | | |
| Human Aortic | 437Z016.2 | C-12533 | PromoCell | Smooth Muscle Cell Growth |
| Smooth Muscle | 453Z011.6 | | | Medium 2 (C-22062) |
| Cells (HAoSMC) | | | | |
| Human Small | 467Z033 | C-12642 | PromoCell | Small Airway Epithelial Cell |
| Airway Epithelial | 467Z025.2 | | | Growth Medium (C-21070) |
| Cells (HSAEpC) | 20TL119096 | CBS- | Lonza | SAGM ™ Small Airway |
| | | 46647A | | Epithelial Cell Growth Medium |
| | | | | BulletKit ™ CC-3118 |
| Human Bronchial | 0000613768 | CC-2576 | Lonza | SmGM ™- 2 Smooth Muscle |
| Smooth Muscle | | | | Cell Growth Medium -2 |
| Cells (HBSMC) | | | | BulletKit ™ (CC-3182) |
| Normal Human | 0000474577 | CC-2586 | Lonza | MGM ™-4 Melanocyte Growth |
| Epidermal | 0000495833 | CC-2504 | | Medium-4 BulletKit ™ (CC- |
| Melanocytes | | | | 3249) |
| (NHEM) | | | | |
| Human Cervical | 70029809 | PCS-480- | ATCC | Cervical Epithelial Cell Basal |
| Epithelial Cells | | 011 | | Medium (PCS-480-032) + |
| (HCerEpC) | | | | Cervical Epithelial Cell Growth |
| | | | | Kit (PCS-480-042) |
| | 06226 | FC-0080 | Lifeline Cell | ReproLife ™ CX Basal |
| | | | Technology | Medium (LM-0055) + |
| | | | | ReproLife CX LifeFactors Kit |
| | | | | (LS-1116) |
| Human Cardiac | 475Z017.2 | C-12810 | PromoCell | Myocyte Growth Medium (C- |
| Myocytes (HCM) | 470Z011.9 | | | 22070) |
| | 472Z002.3 | | | |
| Human White | 465Z008.3 | C-12735 | PromoCell | Preadipocyte Growth Medium |
| Preadipocytes- | 454Z006 | | | (C-27410) |
| subcutaneous | 467Z014 | | | Preadipocyte Differentiation |
| (HWP) | | | | Medium (C-27436) |
| | | | | Adipocyte Nutrition Medium |
| | | | | (C-27438) |
| iCell ® Astrocytes, | 106167 | C1037 | Fujifilm | DMEM/F-12, HEPES |
| 01434 | | | Cellular | (ThermoFisher 11330) + |
| | | | Dynamics | FBS + N-2 supplement |
| | | | | (ThermoFisher 17502048) |
| Astrocytes | 0000672445 | CC-2565 | Lonza | AGM ™ Astrocyte Growth |
| | | | | Medium BulletKit ™ (CC-3186) |
| Hepatocytes | HH1052 | 82006 | In Vitro | UCRM (81015) and UPCM |
| | HH1165 | | ADMET | (81016) |
| | HH1150 | | Laboratories | |
| Human Hepatic | 300076-3 | 10HU-210 | iXCells | Stellate Cell Growth Medium |
| Stellate Cells | | | Biotech | (MD-0014) |
| (HHSteC) | | | | |
| Human Skeletal | 4820 | 3500 | ScienCell | Skeletal Muscle Cell Medium |
| Muscle Cells | 25571 | | | (SkMCM, 3501) |
| (HSkMC) | | | | |
| Retinal Pigment | 23522 | 6540 | ScienCell | Epithelial Cell Medium (4101) |
| Epithelial Cells | | | | |
| (RPEC) | | | | |
| Human Prostate | 06335 | FC-0038 | Lifeline Cell | ProstaLife basal medium (LM- |
| Epithelial Cells | 02321 | | Technology | 0017) + ProstaLife LifeFactors |
| (HPrEpC) | | | | kit (LS-1072) |
| Human Mammary | 70043304 | PCS-600- | ATCC | Mammary Epithelial Cell |
| Epithelial Cells | | 010 | | Basal Medium (PCS-600-030) + |
| (HMEpC) | | | | Mammary Epithelial Cell |
| | | | | Growth Kit (PCS-600-040) |
| | 03025 | FC-0063 | Lifeline Cell | MammaryLife ™ Basal |
| | | | Technology | Medium (LM-0041) + |
| | 02904 | FC-0065 | Lifeline Cell | MammaryLife LifeFactors Kit |
| | | | Technology | (LS-1088) |
| Human Uterine | 02614 | FC-0075 | Lifeline Cell | VascuLife ® Basal Medium |
| Smooth Muscle | | | Technology | (LM-0002) + VascuLife SMC |
| Cells (HUtSMC) | | | | LifeFactors (LS-1040) |
| iCell ® | 105979 | C1012 | Fujifilm | iCell Neural Base Medium 1 |
| GABANeurons, | | | Cellular | (M1010) + iCell Neural |
| 01434 | | | Dynamics | Supplement A (M1032) |
| Human aortic | 482Z007.2 | C-12271 | PromoCell | Endothelial Cell Growth |
| endothelial cells | 472Z002.1 | | | Medium MV (C-22020) |
| (HAoEC) | 473Z030.1 | | | |
| Human pulmonary | 482Z007.13 | C-12241 | PromoCell | Endothelial Cell Growth |
| artery endothelial | 458Z016.15 | | | Medium (C-22010) |
| cells (HPAEC) | 483Z028.2 | | | |
| iCell | 106318 | C1006 | Fujifilm | iCell Cardiomyocytes |
| Cardiomyocytes, | | | Cellular | maintenance medium |
| 01434 | | | Dynamics | (M1003) iCell Cardiomyoctes |
| | | | | plating medium (M1001) |
| Macrophages | D179395 | | Hemacare | ImmunoCult ™-SF |
| (PBMC-derived) | 12429 | | AllCells | Macrophage Medium |
| | | | | (StemCell Technologies |
| | | | | #10961) |
| Human Renal | 26092 | 4100 | ScienCell | Epithelial Cell Medium (4101) |
| Proximal Tubular | | | | |
| Epithelial Cells | | | | |
| (HRPTEpC) | | | | |
| Normal human | 489Z017.1 | C-12003 | PromoCell | Keratinocyte Growth Medium |
| epidermal | 488Z006.2 | C-12001 | | 2 (C-20011) |
| keratinocytes | | | | |
| (NHEK) | | | | |
| Human Ovarian | 24311 | 7330 | ScienCell | Fibroblast medium (Cat No |
| Fibroblasts | | | | #2301) |
| Human Bronchial | 20TL119094 | CBS- | Lonza | BEGM ™ Bronchial Epithelial |
| Epithelial Cells | | 46647B | | Cell Growth Medium |
| | | | | BulletKit ™ CC-3170 |
| Human Renal | 8963 | 4120 | ScienCell | Epithelial Cell medium (Cat |
| Epithelial Cells | | | | No #4101) |
| Human Bladder | 05972 | FC-0079 | Lifeline Cell | UroLife ™ Basal Medium (LM- |
| Epithelial Cells | | | Technology | 0054) + UroLife LifeFactors |
| | | | | (LS-1115) |

Text in bold in the ‘Lot/batch number’ column was used to identify the lot/batch of the target
cells in the graphs. PBMCs were isolated from whole Leukopaks purchased from the vendors
indicated in the table.

T cell media

| Media | Components | Stock (mL) | Catalog Number |
|---|---|---|---|
| “Cytokine-free T cell | X-VIVO 15 | 1000 | Lonza#04-418Q |
| medium” | Human Serum (Heat- | 50 | Sigma-Aldrich#H3667-100 |
| | Inactivated) | | |
| | Pen Strep | 10 | Gibco#15149-122 |
| | GlutaMAX (100x) | 10 | FisherScientific#35050061 |
| “T cell medium with | Cytokine-free T cell medium | 400 | NA |
| Cytokines” | Human IL-2 (5 μg/mL) | 2 | Sigma- |
| | | | Aldrich#11147528001 |
| | Human IL-7 (10 μg/mL) | 0.2 | R&D Systems#207-IL-025 |

Media and supplements

| | | Catalog |
|---|---|---|
| Media | Vendor | Number |
| X-VIVO 15 | Lonza | 04-418Q |
| Human Serum (Heat-Inactivated) | Sigma-Aldrich | H3667-100 |
| FBS, heat-inactivated | Thermofisher Scientific | A3840001 |
| GlutaMAX (100x) | Fisher Scientific | 35050061 |
| Penicillin-Streptomycin | Gibco | 15140-122 |
| RPMI medium 1640 | Gibco | A10491-01 |
| DMEM | Gibco | 11965 |
| EMEM medium | ATCC | 30-2003 |
| IMDM medium | ATCC | 30-2005 |
| Endothelial Cell Growth Medium 2 | PromoCell | C-22011 |
| Endothelial Cell Growth Medium | PromoCell | C-22010 |
| Endothelial Cell Growth Medium MV | PromoCell | C-22020 |
| Keratinocyte Growth Medium 2 | PromoCell | C-20011 |
| Fibroblast Growth Medium | PromoCell | C-23010 |
| Fibroblast Growth Medium 2 | PromoCell | C-23020 |
| Fibroblast Growth Medium 3 | PromoCell | C-23025 |
| Smooth Muscle Cell Growth Medium 2 | PromoCell | C-22062 |
| Small Airway Epithelial Cell Growth Medium | PromoCell | C-21070 |
| SAGM ™ Small Airway Epithelial Cell Growth Medium | Lonza | CC-3118 |
| BulletKit ™ | | |
| SmGM ™- 2 Smooth Muscle Cell Growth Medium -2 | Lonza | CC-3182 |
| BulletKit ™ | | |
| BEGM ™ Bronchial Epithelial Cell Growth Medium | Lonza | CC-3170 |
| BulletKit ™ | | |
| MGM ™-4 Melanocyte Growth Medium-4 BulletKit ™ | Lonza | CC-3249 |
| Cervical Epithelial Cell Basal Medium | ATCC | PCS-480-032 |
| Cervical Epithelial Cell Growth Kit | ATCC | PCS-480-042 |
| ReproLife ™ CX Basal Medium | Lifeline Cell Technology | LM-0055 |
| ReproLife CX LifeFactors Kit | Lifeline Cell Technology | LS-1116 |
| Myocyte Growth Medium | PromoCell | C-22070 |
| Preadipocyte Growth Medium | PromoCell | C-27410 |
| Preadipocyte Differentiation Medium | PromoCell | C-27436 |
| Adipocyte Nutrition Medium | PromoCell | C-27438 |
| DMEM/F-12, HEPES | ThermoFisher | 11330 |
| N-2 supplement | ThermoFisher | 17502048 |
| AGM ™ Astrocyte Growth Medium BulletKit ™ | Lonza | CC-3186 |
| Universal Cryopreserved Hepatocyte Recovery | In Vitro ADMET | 81015 |
| Medium (UCRM) | Laboratories | |
| Universal Hepatocyte Primary Cell Plating Medium | In Vitro ADMET | 81016 |
| (UPCM) | Laboratories | |
| Stellate Cell Growth Medium | iXCells Biotechnology | MD-0014 |
| Skeletal Muscle Cell Medium | ScienCell | 3501 |
| Epithelial Cell Medium | ScienCell | 4101 |
| ProstaLife basal medium | Lifeline Cell Technology | LM-0017 |
| ProstaLife LifeFactors kit | Lifeline Cell Technology | LS-1072 |
| UroLife ™ Basal Medium | Lifeline Cell Technology | LM-0054 |
| UroLife LifeFactors | Lifeline Cell Technology | LS-1115 |
| Mammary Epithelial Cell Basal Medium | ATCC | PCS-600-030 |
| Mammary Epithelial Cell Growth Kit | ATCC | PCS-600-040 |
| MammaryLife ™ Basal Medium | Lifeline Cell Technology | LM-0041 |
| MammaryLife LifeFactors Kit | Lifeline Cell Technology | LS-1088 |
| VascuLife ® Basal Medium | Lifeline Cell Technology | LM-0002 |
| VascuLife SMC LifeFactors | Lifeline Cell Technology | LS-1040 |
| iCell Neural Base Medium 1 | Fujifilm Cellular | M1010 |
| | Dynamics | |
| iCell Neural Supplement A | Fujifilm Cellular | M1032 |
| | Dynamics | |
| ImmunoCult ™-SF Macrophage Medium | StemCell Technologies | 10961 |
| Fibroblast Medium | ScienCell | 2301 |

Reagents

| | | Catalog |
|---|---|---|
| Reagent | Vendor | Number |
| DPBS without CaCl2, MgCl2 | Thermofisher | 14190-144 |
| | Scientific | |
| TrypLE | Thermofisher | 1260-08 |
| | Scientific | |
| ViaStain AOPI Staining Solution | Nexcelom | CS2-0106- |
| | Bioscience | 25mL |
| DMSO | Sigma Life Science | D2650-100mL |
| Interleukin-2, recombinant human (rhIL-2) | Sigma-Aldrich | 1147528001 |
| Interleukin-7, recombinant human (rhIL-7) | R&D Systems | 207-IL-025 |
| Simple Plex Cartridge for 72 samples, 1 analyte Human - | Biotechne, Protein | SPCKB-PS- |
| IFNg (3rd Gen) | Simple | 002574 |
| RNeasy Plus Mini Kit | Qiagen | 74124 |
| Tapestation RNA ScreenTape Ladder | Agilent | 5067-5578 |
| Tapestation RNA ScreenTape Sample Buffer | Agilent | 5067-5577 |
| Qubit RNA BR | ThermoFisher | Q10210 |
| NEBNext Ultra II Directional RNA Library Prep Kit for | NEB | E7760S |
| Illumina | | |
| SPRIselect | Beckman Coulter | B23319 |
| Qubit dsDNA HS | ThermoFisher | Q32854 |
| Tapestation High Sensitivity D5000 Reagents | Agilent | 5067-5593 |
| Tapestation High Sensitivity D5000 Ladder | Agilent | 5067-5594 |
| PhiX Control v3 | Illumina | FC-110-3001 |
| NextSeq 1000/2000 P1 Reagents (300 Cycles) | Illumina | 20050264 |
| NextSeq 1000/2000 P2 Reagents (200 Cycles) v3 | Illumina | 20046812 |
| NextSeq 2000 P3 Reagents (300 Cycles) | Illumina | 20040561 |
| Anti-Human CD14 Alexa Fluor 488 ® (clone: M5E2) | Biolegend | 301811 |
| Anti-human CD68 Antibody,) APC (Clone Y1/82A) | Biolegend | 333809 |
| Anti-human HLA-A, B, C PE (clone W6/32) | Biolegend | 311406 |

IFN-γ levels were determined in the culture supernatants as a measure of T cell reactivity. Bulk RNA sequencing was performed on all target cells to determine the expression of PRAME, and of putative off-targets including EFNA1, PLA2G4E and SLC26A1. The TCR-T cells produced from three independent batches were systematically tested against each target cell.

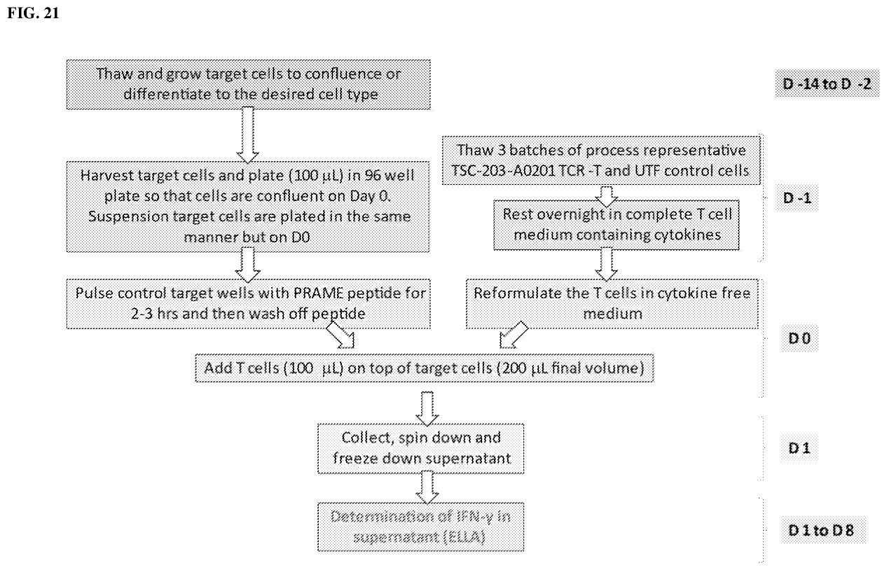

FIG. 21 outlines steps and timelines of the cytokine assay used to test off-tumor reactivity of TSC-203-A0201 TCR-T cells. Briefly, process-representative TSC-203-A0201 TCR-T cells and donor-matched UTF control T cells from three independent donors were co-cultured with HLA-A*02:01⁺ cancer cell lines, primary human cells, and iPSC-derived human cells from healthy donors for 20-28 hours. Culture supernatants were collected and evaluated for IFN-γ levels as a measure of T cell reactivity to the target cells. The reactivity of TSC-203-A0201 for the primary target cells was compared to multiple positive and negative controls. Multiple negative controls were used to establish the baseline IFN-γ levels in the assay. These included (1) donor-matched UTF T cells co-cultured with the same target cells, (2) TSC-203-A0201 TCR-T cells and UTF T cells co-cultured with negative control cell line CaSki, (3) TSC-203-A0201 and UTF T cells cultured alone in the absence of any targets, and (4) target cells cultured alone in the absence of any T cells. Positive controls were included to ensure that TSC-203-A0201 TCR-T cells and target cells used in the assay were functional. Target cells pulsed with the PRAME peptide SLLQHLIGL were co-cultured with TSC-203-A0201 TCR-T cells to ensure that the target cells were healthy and express sufficient levels of HLA to activate TCR-T cells in response to the cognate peptide/MHC (pMHC). TSC-203-A0201 TCR-T cells were co-cultured with positive control cell line SK-MEL-5 to establish IFN-7 levels in response to endogenous SK-MEL-5 and HLA expression.

Target cells and TCR-T cells were thawed. Cancer cell lines were thawed in a 37° C. water bath and washed once with their respective cell culture medium to remove cryopreservation reagents. Cells were subsequently resuspended in their respective cell culture medium and cultured following standard procedures in 175 cm² flasks (adherent target cells) at 37° C., and 5% CO₂. Cells were kept at a sub-confluent state, in the exponential growth phase and passaged once or twice a week as needed. The cancer cell lines were maintained in culture for at least one passage, and no longer than 4 weeks prior to the initiation of the co-culture with TCR-T cells.

Primary human cells and iPSC-derived cells were thawed and seeded in appropriate media Table 13 in tissue culture flasks/plates as per the manufacturer's recommendations. Media was replaced on primary cells the day after thaw with appropriate fresh media. Media was replaced thereafter as per the manufacturer's recommendations until the cells reached confluency and were ready for coculture with TCR-T cells. Human white preadipocytes (HWP) were differentiated to adipocytes for 2 weeks and then used as target cells in the coculture with TCR-T cells. Human cardiac myocytes (HCM) were allowed to mature in culture for 2 weeks before testing them as targets in the coculture. Peripheral blood mononuclear cells (PBMC) were used as targets immediately post-thaw, iCell GABA Neurons, iCell Astrocytes and iCell Cardiomyocytes were plated directly in 96-well plates post-thaw and used for coculture after a week. Hepatocytes were thawed and plated directly in 96 well flat bottom plates at the vendor-recommended density. Macrophages were differentiated from monocytes isolated from PBMCs. Macrophages were stained for CD14 and CD68 using flow cytometry to confirm differentiation.

TCR-T cells were thawed in a manner where on day −1, T cells were thawed in a 37° C. water bath and washed with cytokine-free T cell medium to remove cryopreservation reagents prior to being resuspended in T cell medium containing cytokines. T cells were seeded in G-Rex® 6-well plates at a density of 1 E6-2 E6 viable cells/mL and allowed to recover in a humidified incubator at 37° C., and 5% CO₂ for 24 hours.

Adherent target cancer cell lines were plated one day prior to setting up the coculture. Cells were resuspended in respective media at a density of 0.5 E6 cells/mL. Target cells were plated in 100 μL of their respective medium (Error! Reference source not found.Table 13) in 96 well flat bottom plates at 50,000 cells per well and allowed to attach overnight in the 37° C. incubator.

Primary and iPSC-derived cells were harvested as per the vendor's recommendations. Cells were resuspended in respective media at a density of 0.25 E6 cells/mL. Target cells were plated in 100 μL of their respective medium (Table 13) in 96 well flat bottom plates at 25,000 cells per well and allowed to attach overnight in the 37° C. incubator. Hepatocytes were thawed and plated directly in 96 well flat bottom plates at the vendor-recommended densities on day −1. Likewise. HWP, HCM, iCell Astrocytes, iCell GABA Neurons and iCell Cardiomyocytes were plated directly at vendor-recommended seeding densities.

Cancer cell lines (647-V, BICR 56, CAMA-1, LS1034, NCI-H1666, SK-MEL-5) and primary cells (NHEM lot 5833; HCM lots Z017, Z011, Z002; iCardiomyocytes donor 01434. HRPTEpC lot 26092; Macrophages derived from donors D395 and D439 and HOF lot 24311) were separately collected in tubes (2 E5-3 E6 cells each) and following a PBS wash, were flash frozen as pellets for RNA sequencing to determine expression of off-targets of the therapeutic TCR in TSC-203-A0201 TCR-T cells.

Co-culture was set up on Day 0. PRAME₄₂₅₋₄₃₃ peptide was diluted to a final concentration of 200 ng/mL in cytokine-free T cell medium. On the day of coculture, cells from relevant wells were peptide-pulsed with 100 μL peptide-containing media for a final peptide concentration of 100 ng/mL and incubated for 2-3 hours at 37° C., 5% CO₂. Post incubation, cells were gently washed 3 times with cytokine-free T cell media and 100 μL of target cell media was added. The plates were observed under the microscope to confirm that the cells remained attached unless described otherwise in the results described herein.

TSC-203-A0201 TCR-T cells and donor-matched UTF control T cells were collected after overnight recovery, resuspended in cytokine-free T cell media and 100 μL of 40,000 CD34⁺/Q⁺TSC-203-A0201 TCR-T cells were added to the appropriate wells for coculture with target cells. The same total numbers of corresponding UTF control T cells were added for any given T cell donor. For the ‘T cell only’ control, 100 μL of TCR-T cell suspension was plated along with 100 μL of cytokine-free T cell media.

For the ‘target cell only’ wells, 100 μL of cytokine-free T cell media was added to target cell media. Cocultures were incubated at 37° C., 5% CO₂ for 20-28 hrs.

Ella-based assessment of TCR reactivity for their cognate pMHC was performed on day 1-8. IFN-γ secretion was measured to evaluate the reactivity of TCR-T cells and donor-matched UTF T cells to cancer cell lines, primary and iPSC-derived human cells. After 20-28 h of coculture at 37° C., 5% CO₂, supernatants were collected and frozen at −80° C. After thawing the supernatants, IFN-γ analysis was performed on the Protein Simple ELLA (automated ELISA platform) according to the manufacturer's instructions. The raw data were exported and graphed in GraphPad Prism (v9.4.1).

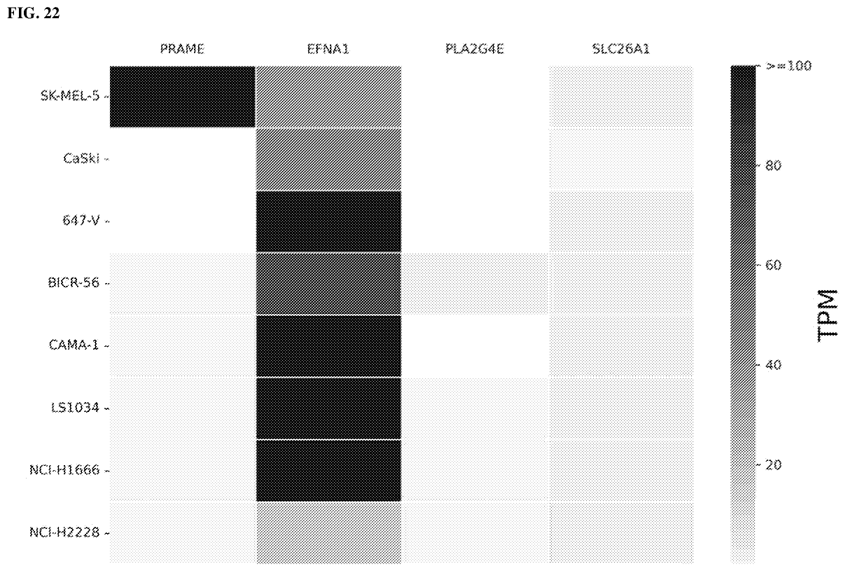

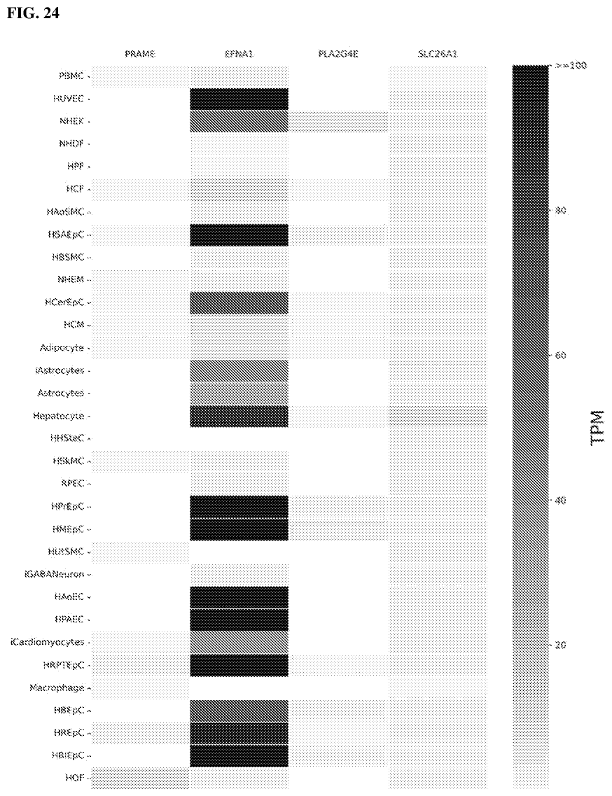

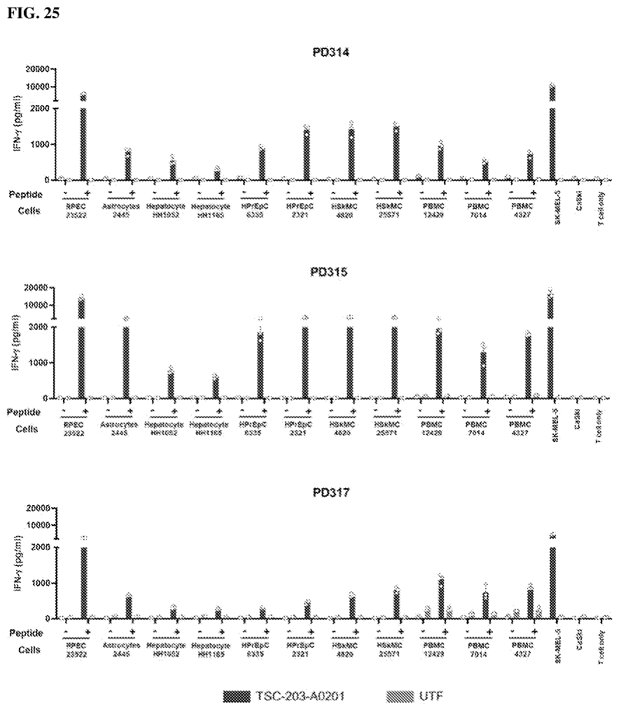

Total RNA was extracted from cell pellets using RNeasy Plus Mini kits. Following manufacturer's instructions, poly-adenylated RNA was isolated from up to 1 μg total RNA using NEBNext Poly(A) mRNA Magnetic Isolation Module and directional RNAseq libraries were constructed using NEBNext Ultra II Directional RNA Library Prep Kit for Illumina (NEB, E7760S). Libraries were then sequenced paired-end (100 bp×100 bp) on an Illumina NextSeq2000 machine. Fastq files were preprocessed prior to alignment and gene expression calculation. The tools used for the preprocessing were Trimmomatic to cut adapter and Illumina-specific sequences from the read. Alignment was performed using hisat2 and annotated to the GRCh38 reference. The GRCh38 reference was taken from Gencode, Release 30 (GRCh38.p12). The resulting alignment files were coordinate sorted using Samtools before gene expression calculation by subReads featureCounts function. Feature counts were converted to TPM (Transcript Per Million) by first dividing the read counts by the length of each gene in kilobases to get reads per kilobase (RPK). RPK is summed per sample then divided by 1,000,000 to give a “per million” scaling factor to generate final TPM for each sample. The color scale used in RNAseq heatmaps has TPM values of zero set to white and values above zero follow a continuous color scale up to 100 TPM as per the legend next to each heatmap in FIG. 22 and FIG. 24.

a. Cancer Cell Line Expression of Putative Off-Targets

To demonstrate safety and lack of off-target reactivity, TSC-203-A0201 TCR-T cells and donor-matched UTF control T cells were cocultured with a selected panel of cancer cell lines comprising LS1034. NCI-H1666, BICR 56, CAMA-1, 647-V. NCI-H2228, and CaSki. These cell lines are HLA-A*02:01-positive and express high levels of the putative off-targets of TSC-203-A0201 TCR-T cells (internal RNAseq data).

Briefly, RNAseq was performed on LS1034, NCI-H1666, BICR 56, CAMA-1, 647-V, NCI-H2228, SK-MEL-5 and CaSki cell lines to confirm the expression of the putative off-targets in cancer cell lines used to test the off-target reactivity of TSC-203-A0201 TCR-T cells. As shown in FIG. 22. SK-MEL-5 showed high PRAME expression and hence served as a positive control in this assay to demonstrate that TCR-T cells used in these assays are functional. CaSki had no PRAME expression and served as a negative control target cell line in this assay. PRAME was expressed at less than 0.5 TPM in BICR 56. CAMA-1, LS1034, NCI-H1666 and NCI-H2228 cell lines. However, no reactivity of TSC-203-A0201 TCR-T cells was observed to these cell lines expressing less than 0.5 TPM of PRAME (FIG. 22).

All the tested cancer cell lines expressed the putative off-target EFNA1 ranging from 17.4 to 429.9 TPM. CAMA1 exhibited the highest EFNA1 expression of 429.9 TPM followed by 647-V at 274.3 TPM and NCI-H1666 at 226.4 TPM. NCI-H2228 showed the lowest EFNA1 expression of 17.4 TPM out of all the tested cell lines. Expression of PLA2G4E ranged from 0.01 to 2.04 TPM with BICR 56 expressing the highest levels at 2.04 TPM and LS1034, NCI-H1666 and NCI-H2228 expressing less than 0.3 TPM. No expression of PLA2G4E expression was detected in SK-MEL-5, CaSki, 647-V and CAMA-1 cell lines. SLC26A1 expression ranged from 0.2 TPM to 2.7 TPM. NCI-H2228 expressed highest levels of SLC26A1 at 2.7 TPM followed by LS1034 at 2.6 TPM and CAMA1 at 2.3 TPM. All the other tested cancer cell lines expressed less than 2 TPM of the putative off-target SLC26A1.

b. Lack of TSC-203-A0201 TCR-T Cell Reactivity to Putative Off-Targets

To evaluate reactivity of TSC-203-A0201 TCR-T cells to putative off-targets, 3 hatches of process-representative materials (PD314, PD315, and PD317) were cocultured with LS1034, NCI-H1666, BICR 56. CAMA-1, 647-V, NCI-H2228, SK-MEL-5 and CaSki cell lines and IFN-γ levels were determined in the culture supernatants as a measure of T cell reactivity.

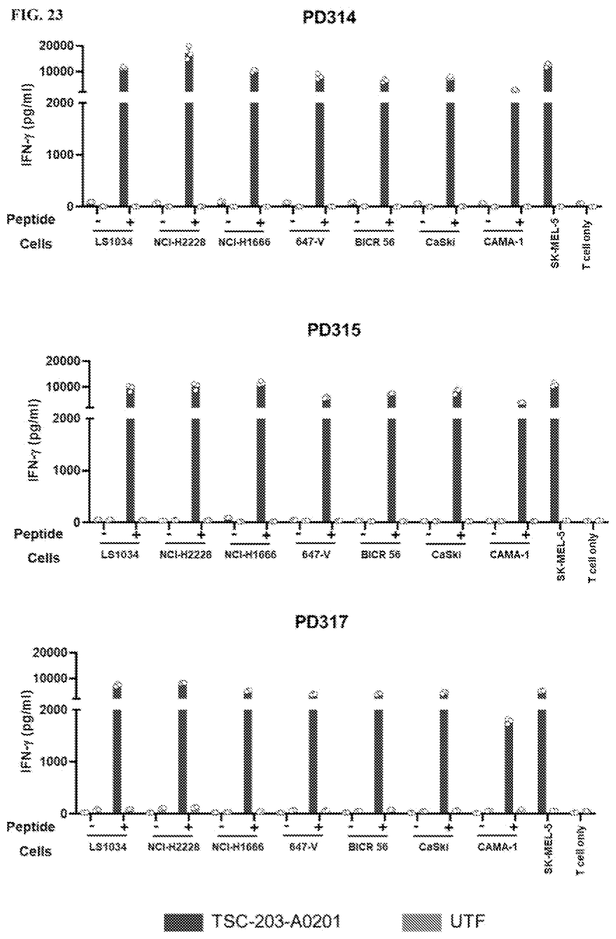

FIG. 23 shows that no reactivity of TSC-203-A0201 TCR-T cells was observed for any of the cancer cell lines tested and that the positive and negative controls performed as expected. All batches of TSC-203-A0201 TCR-T cells secreted IFN-γ in response to all PRAME₄₂₅₋₄₃₃ peptide-pulsed target cell lines. In the absence of exogenous peptide-pulse, baseline levels of IFN-γ were observed for all batches of TSC-203-A0201 TCR-T cells similar to those observed for control UTF T cells indicating lack of reactivity of the therapeutic T cells to the cancer cell lines tested. Altogether, these cancer cell lines express EFNA1, PLA2G4E and SLC26A1 indicating lack of reactivity of TCR-T cells to physiological levels of these proteins.

c. Primary and iPSC-Derived Cell Expression of Putative Off-Targets

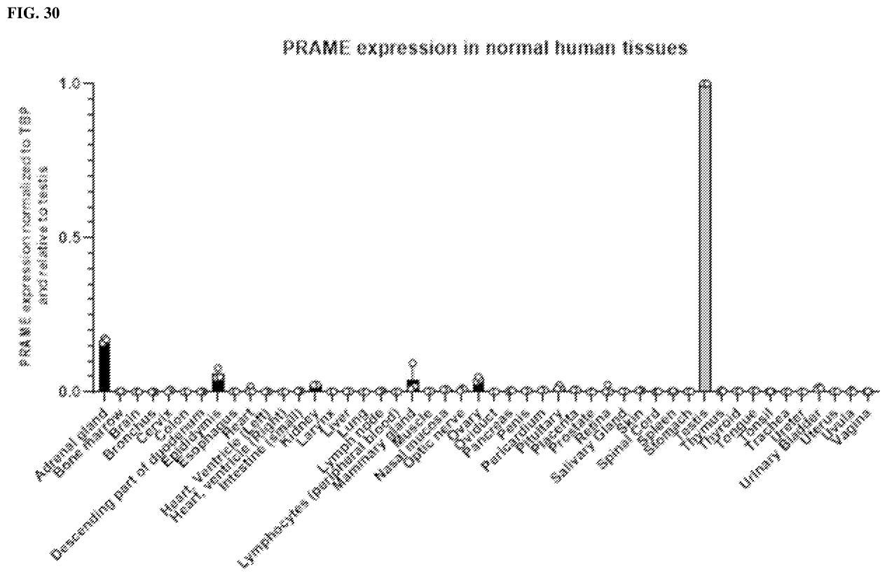

Bulk RNA sequencing was performed on target cells to determine expression of PRAME and the identified putative off-targets of TSC-203-A0201. As shown in FIG. 24, varying levels of expression of EFNA1, PLA2G4E and SLC26A1 were detected in the cell types tested in this study. Internal RNASeq data showed that out of all the primary cells tested, the highest EFNA1 expression of 784 TPM was observed in HUVECs. Expression of EFNA1>100 TPM was also observed in HAoEC (237 TPM), HPAECs (220.3 TPM), HBIEpCs (189.3 TPM), HRPTEpCs (151.9 TPM), HSAEpCs (108.2 TPM), HPrEpCs (108.6 TPM) and HMEpCs (118.3 TPM). All other primary cell types exhibited EFNA1 expression ranging from 0 to 100 TPM. HHSteCs, HUtSMC and macrophages showed no expression (0 TPM) of EFNA1. NHEKs showed the highest PLA2G4E expression at 6.3 TPM, followed by HMEpC that expressed 3.14 TPM. All other primary cells tested exhibited less than 1 TPM of PLA2G4E expression. All primary cell types tested exhibited SLC26A1 expression ranging from 0.36 to 8.9 TPM. Hepatocytes showed the highest SLC26A1 expression of 8.9 TPM and macrophages showed the lowest SLC26A1 expression of 0.36 TPM. PRAME expression is largely restricted to the testis; however, lower yet detectable levels are observed in the kidney, adrenal glands and the ovaries by qPCR (FIG. 30 in Example 8). Consistent with the qPCR data, expression of PRAME was observed in HRPTEpCs (2.7 TPM) and HREpCs (1.5 TPM) as well as HOF (8.0 TPM). However, as shown below, no reactivity of the three batches of TCR-T cells was detected upon coculture with these cells.

d. Lack of TSC-203-A0201 TCR-T Cell Off-Tumor Reactivity to Primary and iPSC-Derived Cells

The reactivity of TSC-203-A0201 was evaluated against a selected panel of 60 HLA-A*02:01-positive healthy primary human cells and iPSC-derived cells. This panel included cells from various lineages such as epithelial, mesenchymal, endothelial, fibroblastic, muscle cells derived from multiple vital and non-vital organs, reproductive and non-reproductive organs, derived from male and female donors comprising the organs/tissues that are traditionally assessed during toxicology studies.

As shown in FIG. 24, varying levels of expression of EFNA1, PLA2G4E and SLC26A1 were detected in the cell types tested in this study. These data indicate that the primary cell panel includes an extensive collection of cell types that endogenously express putative off-targets of interest, and any reactivity (or lack thereof) is informative for assessing risk of off-tumor reactivity.

FIG. 25 provides a representative graph demonstrating that no reactivity of three batches of TSC-203-A0201 TCR-T cells and donor-matched untransfected (UTF) control T cells (PD314, PD315, and PD317) co-cultured with (1) a single lot of HLA-A*02:01-positive retinal pigment epithelial cells (RPECs); (2) a single lot of HLA-A*02:01-positive astrocytes; (3) three lots of HLA-A *02:01-positive hepatocytes; (4) two lots of HLA-A*02:01-positive human cervical epithelial cells (HCerEpCs); (5) two lots of HLA-A*02:01-positive Human Skeletal Muscle Cells (HSkMCs), or (6) three lots of HLA-A*02:01-positive Peripheral Blood Mononuclear Cells (PBMCs), respectively, was observed. Similar data and results were obtained for assays in which the batches of TSC-203-A0201 TCR-T cells and donor-matched untransfected (UTF) control T cells (PD314, PD315, and PD317) co-cultured with the other primary and iPSC-derived cell types. No reactivity of TSC-203-A0201 TCR-T cells was observed for essentially all primary and iPSC-derived cell types tested in this study. In addition, the positive and negative controls performed as expected, demonstrating the validity of these assays (see, for example, FIG. 25).

Taken together, the data presented in this study demonstrate that TSC-203-A0201 TCR-T cells show no apparent reactivity to essentially all primary cells tested including cells that endogenously express the putative off-target of the therapeutic TCR. These data indicate no risk of off-tumor reactivity for TSC-203-A0201 and indicate that the reactivity of the TCR-T cells is restricted to cancer cells expressing PRAME and HLA-A*02:01.

### Example 7: Lack of TSC-203-A0201TCR-T Cell Oncogenicity

An in vitro oncogenicity assay was performed to further confirm that manufacture of TSC-203-A0201 (e.g., during translation of transposase mRNA into enzymatic protein within the cell and facilitates the transposition of TSC-203-A0201 npDNA transposon at 5′-TTAA-3′ sites within the host genome) does not result in vector-induced insertional mutagenesis and oncogenic transformation of the TCR-T cells (e.g., that translation of transposase mRNA into enzymatic protein within the cell and facilitates transposition of TSC-203-A0201 npDNA transposon at 5′-TTAA-3′ sites within the host genome) (Wu et al. (2011) Front Med, 5:356-371; Manfredi (2020) Front. Immunol, 11:1689; Nobles et al. (2020) J. Clin. Invest, 130:673-685; Micklethwaite et al. (2021) 138:1391-1405).

To evaluate cytokine-dependency, process-representative TSC-203-A0201 TCR-T cells and donor-matched untransfected (UTF) control T cells were cultured in the presence or absence of cytokines (IL-2 and IL-7) for 5 days and analyzed for cell survival and proliferation. UTF control T cells that have not undergone any electroporation and transposition, and hence, are devoid of any insertional mutagenesis served as controls. A positive control of T cell proliferation was included by stimulating the TSC-203-A0201 TCR-T cells and donor-matched UTF control T cells with ImmunoCult™ (IQ) Human CD3/CD28/CD2 T cell Activator.

The data demonstrate that, similar to donor-matched UTF control cells. TSC-203-A0201 TCR-T cells exhibited lower survival and proliferation when cultured in the absence of cytokines compared to T cell survival and proliferation when cultured in the presence of cytokines. Further, in the absence of cytokines, both proliferation and survival of TSC-203-A0201 TCR-T cells was similar or lower to that of the donor-matched UTF cells. Together these data indicate lack of cytokine-independent survival or (hyper)proliferation of TCR-T cells.

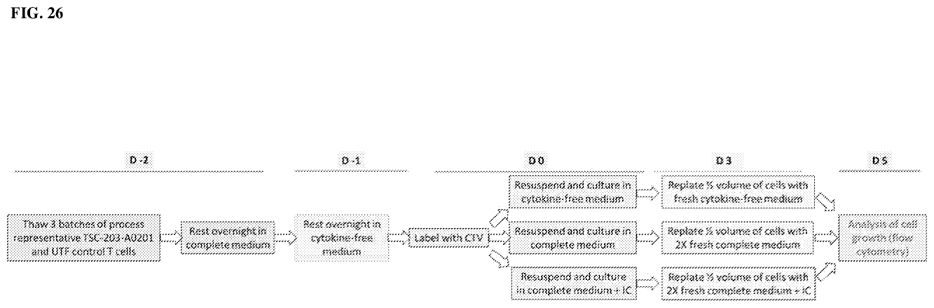

In particular, the oncogenicity assay was performed according to the steps and timeline depicted in FIG. 26. TSC-203-A0201 TCR-T cells and donor-matched UTF T cells were thawed and maintained in complete T cell medium (media with cytokines-Table 14) on day −2.

TABLE 14
Reagents

Prepared T cell media

| | | Volume stock | |
|---|---|---|---|
| Media | Components | (mL) | Catalog Number |
| Cytokine-free T cell | X-VIVO 15 | 1000 | Lonza |
| medium | | | #04-418Q |
| | Human Serum (Heat- | 50 | Sigma-Aldrich |
| | Inactivated) | | #H3667-100 |
| | Pen Strep | 10 | Gibco |
| | | | #15149-122 |
| | GlutaMAX (100x) | 10 | FisherScientific |
| | | | #35050061 |
| | Total | 1070 | n/a |
| Complete T cell | Cytokine-free T cell media | 200 | n/a |
| medium (media | Human IL-2 (5 ug/mL) | 1 | Sigma-Aldrich |
| with cytokines) | | | #11147528001 |
| | Human IL-7 (10 ug/mL) | 0.1 | R&D Systems |
| | | | #207-IL-025 |
| | Total | 201.1 | n/a |

Media composition for oncogenicity assay

| | | Cytokines (IL-2 & IL-7) | | ImmunoCult ™ | |
|---|---|---|---|---|---|
| Media to prepare | | 2x concentration preparation | | | |
| T cell media #1 | Cytokine-free T cell media | − | NA | − | NA |
| (10 ml) | | | | | |
| T cell media #2 | Complete T cell media (media | + | 100 μL IL-2 | − | NA |
| (10 ml) | with cytokines) | | 10 μL IL-7 | | |
| T cell media #3 | Positive control T cell media | + | 100 μL IL-2 | + | 40 μL |
| (10 ml) | (complete T cell media with | | 10 μL IL-7 | | |
| | cytokines + IC) | | | | |

Media and supplements

| Media | Vendor | Catalog Number |
|---|---|---|
| X-VIVO 15 | Lonza | 04-418Q |
| Human Serum (Heat-Inactivated) | Sigma-Aldrich | H3667-100 |
| HI FBS | Gibco | Q38400-01 |
| FBS, heat-Inactivated | Thermofisher Scientific | A3840001 |
| GlutaMAX (100x) | Fisher Scientific | 35050061 |
| Penicillin-Streptomycin | Gibco | 15140-122 |

Reagents

| Reagent | Vendor | Catalog Number |
|---|---|---|
| CellTraceTM Violet proliferation kit | Thermofisher Scientific | C34557 |
| Fixable Viability Dye eFluor ™ 660 | eBioscience | 65-0864-14 |
| ViaStain AOPI Staining Solution | Nexcelom Bioscience | CS2-0106-25mL |
| EasySep ™ Buffer | STEMCELL Technologies | 20144 |
| Interleukin-2, recombinant human (rhIL-2) | Sigma-Aldrich | 1147528001 |
| Interleukin-7, recombinant human (rhIL-7) | RD Systems | 207-IL/CF |
| ImmunoCult ™ | STEMCELL Technologies | 10970 |

Specifically, T cells were thawed in a 37° C. water bath and washed twice with cytokine-free T cell medium (Table 12) to remove cryopreservation reagents prior to being resuspended in T cell medium containing cytokines. T cells were seeded in a G-REX® 6-well plate at a density of 1-2 E6 viable cells/ml and allowed to recover in a humidified incubator at 37° C. and 5% CO₂ for 24 hours.

After allowing the T cells to recover for 24 hours, the T cells were washed in cytokine-free culture medium and then maintained in cytokine-free culture medium on day −1. T cells were seeded in a G-REX® 6-well plate at a density of 1-2 E6 viable cells/ml and allowed to rest in a humidified incubator at 37° C. and 5% CO₂ fir 24 hours prior to oncogenicity evaluation.

After allowing the T cells to rest for an additional 24 hours, the T cells were stained with CTV (cell trace violet) proliferation dye on Day 0 and were cultured in the following media: 1) cytokine-free medium (absence of IL-2 and IL-7); 2) complete T cell medium (presence of IL-2 and IL-7); and 3) complete T cell medium with Immunocult™ (IC), added as a positive control to stimulate proliferation.

Specifically, on day 0 (48 hours after thaw and recovery), TSC-203-A0201 TCR-T cells and donor-matched UTF control T cells were washed twice in cytokine-free T cell medium, 4 E6 viable cells were stained with CTV proliferation dye at a concentration of 2.5 μM in 1 ml of PBS for 10 minutes at room temperature protected from light. After washing twice with cytokine free T cell medium, 1 E5 viable T cells were then seeded as triplicates into 96-well U-bottom in 200 μl of media. To help reduce evaporation, 200 μl of PBS was added to each well along the border of experimental wells. Cells were confirmed to express similar levels of CTV staining as determined by flow cytometry.

On day 3, half of the T cells were passaged in the appropriate media. Specifically, T cells in the 96-well U-bottom plate were resuspended and half of the cells were transferred to a new 96-well U-bottom plate and passaged in the appropriate 2× media. Cells were cultured for an additional 2 days and were analyzed by flow cytometry.

On day 5, the numbers of viable cells and proportion and numbers of proliferated T cells were determined by flow cytometry. Specifically, T cells were harvested, washed with PBS, and stained with fixable viability dye eFlour 660 according to the manufacturer's instructions. T cells were then resuspended in 100 μl/well of EasySep™ buffer with 9.80 E3 Count Bright beads/well. T cells were analyzed by flow cytometry using the CytoFLEX flow cytometer (Beckman Coulter) (acquisition volume: 90 μl, sample flow rate 60 μl/min) and data were analyzed by FlowJo™ (Treestar) (version 10.8.1).

Numbers of viable and proliferating cells were quantified using a gating strategy. Briefly, precision counting beads were first separately gated out using irrelevant fluorescence channels for cell count normalization purposes. Forward scatter area (FSC-A) vs. side scatter area (SSC-A) density plots were then used to identify cells and to exclude debris. Within this ‘T cells’ population, single T cells were separated from doublets using the FSC-height (FSC-H) vs. FSC-Area (FSC-A) density plots. Next, eFlour 660 APC viability dye staining within the ‘single cell’ population distinguished viable eFlour 660-negative T cells from eFlour 660-positive T cells. Proliferating T cell generations were then separated from non-proliferating T cells by a reduced fluorescence intensity of CellTrace Violet (CTV) proliferation dye within the ‘Live Cells’ population. Absolute count of viable cells (viability dye eFlour 660-negative) and proliferating cells were normalized to absolute bead counts. Normalized counts of viable and dividing cells obtained on day 5 were multiplied by two to compensate for passaging the cells on day 3.

Raw data were exported, graphed, and analyzed in GraphPad Prism (version 9.2.0). Statistical differences between values of TSC-203-A0201 TCR-T cells and their donor-matched non-edited UTF control T cells, and between test conditions of culturing cells in the absence of cytokines or in the presence of cytokines were determined by 2-way ANOVA (Sidak correction for multiple comparisons). ****p≤0.0001, ***p≤0.001, **p≤0.01, *p≤0.05, ‘ns’ means not significant p>0.05.

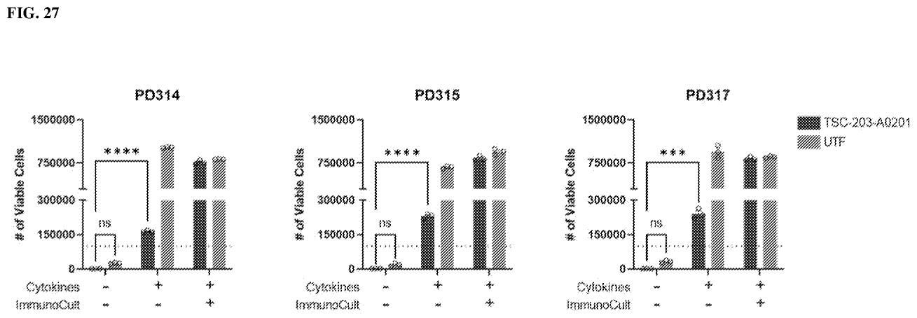

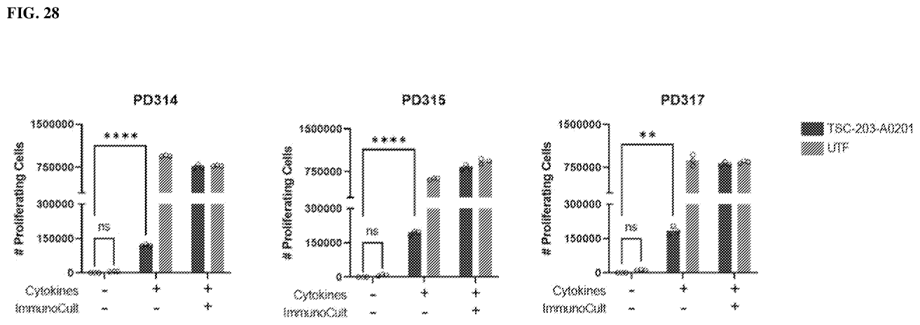

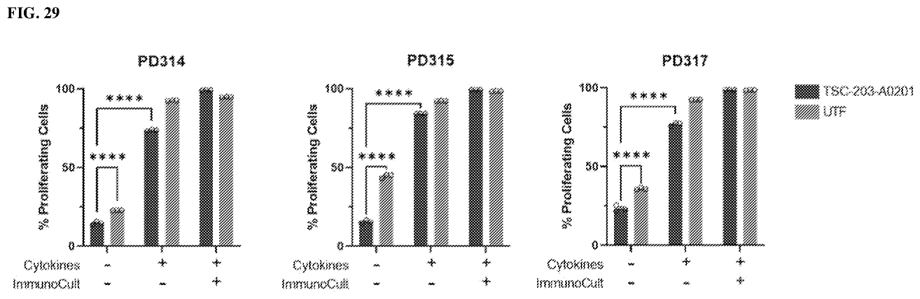

FIG. 27-FIG. 29 show that positive controls exhibit highest levels of proliferation. T cell survival and proliferation in-vitro is dependent on the stimulation through the TCR and/or cytokines. TSC-203-A0201 TCR-T cells and donor-matched UTF control T cells stimulated with ImmunoCult™ (IC) Human CD3/CD28/CD2 T cell Activator in the presence of cytokines served as positive controls for the oncogenicity assay (the “Cytokines+, ImmunoCult™+” condition in FIG. 27-FIG. 29. Under these culture conditions, engineered TCR-T cells and their UTF control counterparts from all 3 batches of material tested expanded significantly, resulting in a high number of viable (FIG. 27) and proliferated cells compared to the initial seeding cell number of 100,0000 cells (FIG. 28). The proportion of proliferating cells was also high with >94% of the viable cells undergoing proliferation during the 5 days of the experiment (FIG. 28 and FIG. 29). These observations confirmed that all batches of TSC-203-A0201 TCR-T cells and donor-matched UTF control T cells used in the assay were viable and functional.

By contrast, negative controls exhibited no hyperproliferation since donor-matched UTF control T cells did not undergo electroporation or transposition (thus devoid of any insertional mutagenesis) and these cells represent an ideal comparator to test the cytokine-dependency for cell survival and proliferation of TSC-203-A0201 TCR-T cells. As expected, UTF control T cells cultured in the presence of cytokines were able to survive and proliferate as demonstrated by high numbers of total viable T cells similar to the trends observed for the total numbers of T cells obtained in the positive control condition (FIG. 27). Of those cells, the trends for proportion and total numbers of cells engaging in cell cycle was also comparable to the trends observed in the positive control culture condition (FIG. 28 and FIG. 29).

In the absence of cytokines, however, the UTF control T cells failed to survive and a significant reduction in numbers of total viable cells was observed when compared to UTF control T cells cultured in the presence of the cytokines on day 5 (FIG. 27). The numbers of viable cells in the absence of cytokines were lower than the day 0 seeding cell number of 100,000 cells, confirming that the cells died. Furthermore, all donors tested in the absence of cytokines exhibited a reduction in the total numbers and proportion of proliferating T cells after 5 days (FIG. 28 and FIG. 29). Taken together, these data indicate that, under the conditions of the experiments, unedited UTF control T cells of the batches tested do not survive and only engage in limited proliferation in the absence of cytokines.

TSC-203-A0201 TCR-T cells showed cytokine-dependent survival and proliferation. TSC-203-A0201 TCR-T cells exhibited limited survival and proliferation in the presence of cytokines (FIG. 27-FIG. 29) with the total numbers of viable and proliferating T cells remaining remarkably lower than what was measured for the donor matched UTF control T cells (FIG. 27 and FIG. 28).

In the absence of cytokines, none of the TSC-203-A0201 batches were able to survive or expand. TSC-203-A0201 TCR-T cells showed a clear reduction in viability and proliferation when compared to TSC-203-A0201 TCR-T cells cultured in the presence of the cytokines (FIG. 27-FIG. 29). Overall, TSC-203-A0201 TCR-T cells presented a level of proliferation (proportion and numbers of proliferating cells) in the absence of cytokines that did not exceed the proliferation of the matching UTF control T cells under similar culture conditions (FIG. 28 and FIG. 29).

Taken together, these data confirm that TSC-203-A0201 TCR-T cells remained dependent on cytokines and TCR-mediated signals to survive and expand similarly to non-edited cells such that the engineering process did not produce insertional mutagenesis resulting in oncogenic transformation of the TCR-T cells.

### Example 8: MAGE-A1 Target Expression in Normal Human Tissues

As described above, PRAME expression is not detected in normal human tissues other than the testis, as has been previously demonstrated. To confirm that MAGE-A1 expression is restricted to cells of the testis, but not other normal tissues, cDNA arrays were purchased from a commercial vendor containing cDNA from 48 different normal (non-cancer) tissue sections and assayed for PRAME and TBP gene expression using a multiplexed PRAME and TBP qPCR assay described below, in technical triplicates.

Specifically, cDNA arrays in 96-well plate format were purchased from Origene and handled according to manufacturer instructions. A master mix consisting of TaqMan® Fast Advanced Master mix (ThermoFisher, cat. #4444557), Nuclease-free water (Invitrogen, cat. #AM9937), PRAME or TBP TaqMan probes, were aliquoted into the 96-well cDNA array plate, 20 μL per well. Gene expression was measured on a QuantStudio 7 and Cq values were quantified, analyzed, and graphed for both Normal tissue (OriGene, cat. #HMRT304) cDNA arrays. Three individual plates were assayed for each array (n=3 technical replicates). Cq values were obtained from the QuantStudio 7 for each cDNA array. Cq values for PRAME was normalized to TBP to obtain the delta CT (ΔCt). The ΔCt values were then quantified using the 2\hat{ }−ΔCt method to indicate levels of PRAME expression relative to levels of TBP. Quantification of individual replicates are graphed with the mean±standard error indicated.

FIG. 30 shows PRAME expression across 48 tissue types measured from the TissueScan—Normal human tissue array. Individual replicates (n=3) of PRAME expression normalized to TBP expression are plotted (bar graph) and the mean and standard error are indicated with black bars. Of the 48 normal tissues examined, only testes exhibited high PRAME expression across 3 technical replicates. PRAME expression was undetectable across 3 technical replicates for 40 tissues. These data concur with the abundant literature supporting that PRAME is an ideal tumor-associated protein and is predominantly absent from non-testes normal tissues (Ikeda et al. (1997) Immunity 6:199-208; Wadelin et al. (2010) Molecular cancer 9:1-10; Paydas et al. (2007) Leukemia research 31:365-369; Lezcano et al. (2018) The American journal of surgical pathology 42:1456; Xu et al. (2020) 53:p.e12770; Gezkin et al. (2017) JAMA ophthalmology 135:541-549).

For 5 tissues (adrenal gland, epididymis, kidney, mammary gland, and ovary), low but detectable signal was observed. The signal from mammary gland was detectable in only 1 out of 3 technical replicates; due to the lack of reproducibility across replicates, this is expected to represent background/false positive signal. The signal from epididymis is expected to reflect potential cross-contamination from testes during gross tissue preparation. With respect to PRAME detected in the other non-testes tissues, low expression of PRAME in the adrenal gland, in the kidney, and in the ovary have also been observed by others (Ikeda et al. (1997) Immunity 6:199-208). When compared to the level of expression in tumor samples, however, non-testes tissues appear to express orders of magnitude lower levels of PRAME.

In addition, as demonstrated in Example 6 where normal human tissues were cocultured with TSC-203-A0201 TCR-T cells, none of the tissues tested triggered reactivity of the TCR-T cells, including ovarian fibroblasts or renal epithelial cells that were confirmed to express low levels of PRAME.

In summary, consistent with prior literature (Ikeda et al. (1997) Immunity 6:199-208; Paydas et al. (2007) Leukemia Res, 31:365-369; Xu et al. (2020) Cell Prolif 53:p.e12770) and publicly available RNA-sequencing data. PRAME expression in normal tissue is largely restricted to testes with low levels detected in isolated tissues such as adrenal gland, kidney, and ovary. These gene expression analyses coupled with safety analyses described herein indicate that PRAME represents a safe target for T cell-based therapies with low risk for on-target/off-tumor reactivity.

### Example 9: Alloreactivity Profiling for TSC-203-A0201

In further support of alloreactivity data described above, alloreactivity profiling of the recombinant TCR used for TSC-203-A0201 TCR-T cells was further evaluated. As described, alloreactivity is the ability of TCRs to recognize allogeneic MHC molecules that were not encountered during the thymic development and will potentially manifest itself clinically as manufacturing failure due to fratricide reactivity, and graft-versus-host disease (GvHD) upon infusion in patient.

Briefly, mechanistically representative TCR-T cells (i.e., CD3⁺ T cells engineered by lentivirus transduction to express the therapeutic TCR used in TSC-203-A0201 along with the CD8αβ co-receptor) were cocultured for 48 hours with target cells (HEK293T cells) expressing individually each of the 110 most common class I MHCs (Maiers et al. (2008) Hum. Immunol, 69:141) in an array format (see Table 155 below for the complete list of MHC-I surveyed in this study and their frequency in the U.S. population). The reactivity of the therapeutic TCR to allogeneic MHC molecules and the resulting cytotoxicity on target cells was assessed by measuring the target cell growth inhibition caused by the TCR-T cells relative to untransduced (UTD) donor control CD3+ T cells.

Mechanistically representative TSC-203-A0201 TCR-T cells elicited strong cell killing on HEK293T cells expressing HLA-A*02:01 and a PRAME 90-mer containing the PRAME-derived epitope SLLQHLIGL, which served as a positive control for the killing assay. HEK293T cells endogenously express PRAME (source: The Human Protein Atlas); the monoallelic cells expressing HLA-A*02:01 also served as a positive control as these cells can endogenously present the PRAME epitope. The highly sensitive, high throughput alloreactivity profiling assay identified that the recombinant TCR used in TSC-203-A0201 lacks reactivity to the majority of the 110 most common MHC alleles. Nevertheless. TSC-203-A0201, based on some prediction criteria, possibly presented a potential risk of alloreactivity to cells expressing the HLA-C alleles C*01:02. C*08:01, C*14:02, C*14:03, C*16:01, or C*16:02, as the coculture of mechanistically representative TSC-203-A0201 TCR-T with the array found that the cells had reactivity to HEK293T cells expressing HLA-C alleles C*01:02, C*08:01, C*14:02, C*14:03. C*16:01, and C*16:02.

Follow-up experiments were conducted to determine whether TSC-203-A0201 presents alloreactivity to these alleles. For these experiments, mechanistically representative TCR-T cells were cocultured with target cell lines naturally expressing the putative allogeneic alleles. T cell reactivity was assessed by measuring IFN-γ secretion. Cancer cell lines positive for HLA-C*01:02, C*08:01, C*14:02, and C*16:01, but negative for either PRAME, and/or HLA-A*02:01, could be identified and tested in coculture experiments with TSC-203-A0201. However, because of its low frequency in the general population, no cell line naturally positive for HLA-C*16:02 could be identified; instead, monoallelic HEK293T cells expressing this allele were used.

These follow-up experiments confirmed that HLA-C*01:02 and HLC-C*16:02 represent two potentially allogeneic alleles for TSC-203-A0201, although the level of reactivity determined was low. In some embodiments encompassed by the present disclosure, subjects treated with TSC-203-A0201 may be negative for HLA-C*01:02 and/or HLC-C*16:02. None of the other alleles originally identified during the high throughput screen were validated in the follow-up experiments, demonstrating negative reactivity for such alleles in practice. Further, when testing the off-tumor reactivity of TSC-203-A0201, primary normal human cells derived from HLA-C*14:02 and HLA-C*16:01 donors were tested and scored negative for reactivity (Example 6) further reinforcing the data presented here. Thus, in some embodiments encompassed by the present disclosures, subjects treated with TSC-203-A0201 may be positive for HLA-C*08:01, C*14:02, C*14:03, and/or C*16:01.

TABLE 15
List of the 110 MHCs surveyed in this study

| HLA-A alleles | freq. | HLA-B alleles | freq. | HLA-C alleles | freq. |
|---|---|---|---|---|---|
| A*02:01:01:01 | 0.2375 | B*07:02:01:01 | 0.1072 | C*07:01:01:01 | 0.1410 |
| A*01:01:01:01 | 0.1256 | B*08:01:01:01 | 0.0902 | C*07:02:01:01 | 0.1298 |
| A*03:01:01:01 | 0.1149 | B*44:02:01:01 | 0.0638 | C*04:01:01:01 | 0.1275 |
| A*24:02:01:01 | 0.0878 | B*35:01:01:01 | 0.0587 | C*06:02:01:01 | 0.0848 |
| A*11:01:01:01 | 0.0551 | B*44:03:01:01 | 0.0518 | C*03:04:01:01 | 0.0752 |
| A*23:01:01:01 | 0.0348 | B*15:01:01:01 | 0.0486 | C*05:01:01:01 | 0.0692 |
| A*29:02:01:01 | 0.0330 | B*51:01:01:01 | 0.0446 | C*16:01:01:01 | 0.0466 |
| A*68:01:01:01 | 0.0304 | B*40:01:01 | 0.0432 | C*02:02:01 | 0.0442 |
| A*26:01:01:01 | 0.0273 | B*18:01:01:01 | 0.0412 | C*03:03:01:01 | 0.0435 |
| A*32:01:01:01 | 0.0266 | B*14:02:01:01 | 0.0293 | C*12:03:01:01 | 0.0410 |
| A*31:01:02:01 | 0.0260 | B*57:01:01:01 | 0.0270 | C*08:02:01:01 | 0.0382 |
| A*30:01:01:01 | 0.0246 | B*27:05:02:01 | 0.0240 | C*01:02:01:01 | 0.0354 |
| A*30:02:01:01 | 0.0209 | B*53:01:01:01 | 0.0237 | C*17:01:01:02 | 0.0204 |
| A*68:02:01:01 | 0.0203 | B*13:02:01:01 | 0.0209 | C*15:02:01:01 | 0.0203 |
| A*33:03:01:01 | 0.0162 | B*38:01:01:01 | 0.0169 | C*14:02:01:01 | 0.0142 |
| A*25:01:01:01 | 0.0139 | B*49:01:01:01 | 0.0169 | C*12:02:01 | 0.0108 |
| A*33:01:01:01 | 0.0129 | B*40:02:01:01 | 0.0166 | C*07:04:01:01 | 0.0104 |
| A*02:06:01:01 | 0.0108 | B*52:01:01:01 | 0.0150 | C*08:01:01:01 | 0.0098 |
| A*02:05:01:01 | 0.0107 | B*58:01:01:01 | 0.0147 | C*03:02:01 | 0.0077 |
| A*74:01:01 | 0.0103 | B*15:03:01:01 | 0.0139 | C*18:01 | 0.0061 |
| A*02:02:01:01 | 0.0088 | B*35:03:01:01 | 0.0138 | C*15:05:01:01 | 0.0061 |
| A*34:02:01 | 0.0065 | B*55:01:01:01 | 0.0132 | C*16:02:01 | 0.0030 |
| A*66:01:01:01 | 0.0050 | B*45:01:01 | 0.0129 | C*08:04:01 | 0.0023 |
| A*36:01 | 0.0046 | B*37:01:01:01 | 0.0109 | C*03:05 | 0.0021 |
| A*29:01:01:01 | 0.0029 | B*42:01:01 | 0.0103 | C*14:03 | 0.0015 |
| A*02:07:01 | 0.0026 | B*50:01:01:01 | 0.0093 | sum | 0.9910 |
| A*03:02:01 | 0.0024 | B*39:01:01:01 | 0.0089 | | |
| A*68:03:01 | 0.0024 | B*35:02:01:01 | 0.0087 | | |
| A*02:03:01 | 0.0020 | B*14:01:01:01 | 0.0083 | | |
| A*01:02 | 0.0016 | B*58:02:01 | 0.0078 | | |
| sum | 0.9787 | B*57:03:01:01 | 0.0071 | | |
| | | B*39:06:01 | 0.0068 | | |
| | | B*15:10:01 | 0.0061 | | |
| | | B*41:01:01:01 | 0.0053 | | |
| | | B*48:01:01:01 | 0.0052 | | |
| | | B*07:05:01:01 | 0.0046 | | |
| | | B*56:01:01:01 | 0.0044 | | |
| | | B*41:02:01:01 | 0.0043 | | |
| | | B*39:05:01:01 | 0.0041 | | |
| | | B*15:17:01:01 | 0.0040 | | |
| | | B*35:08:01:01 | 0.0040 | | |
| | | B*81:01:01 | 0.0038 | | |
| | | B*15:16:01:01 | 0.0038 | | |
| | | B*46:01:01 | 0.0037 | | |
| | | B*35:12:01 | 0.0033 | | |
| | | B*35:17:01 | 0.0028 | | |
| | | B*40:06:01:01 | 0.0028 | | |
| | | B*78:01:01:01 | 0.0025 | | |
| | | B*15:02:01:01 | 0.0023 | | |
| | | B*27:02:01:01 | 0.0023 | | |
| | | B*38:02:01 | 0.0022 | | |
| | | B*13:01:01:01 | 0.0021 | | |
| | | B*47:01:01:02 | 0.0021 | | |
| | | B*54:01:01 | 0.0020 | | |
| | | B*15:18:01:01 | 0.0020 | | |
| | | sum | 0.9660 | | |

In particular, lentivirus was packaged, and lentiviral tier was quantified using the method described above in Example 5. The mechanistically representative TSC-203-A0201 TCR-T cells were generated. In brief, primary CD3 T cells from a single healthy donor were isolated using the StraightFrom Leukopak CD3 Microbead Kit (Miltenyi Biotec) according to the manufacturer's protocol. Isolated cells were frozen in CryoStor® CS10 (Stem Cell Technologies) and stored in liquid nitrogen until use. On Day −1, CD3+ T cells were thawed, washed with complete T cell medium (RPMI 1640 supplemented with 10% heat-inactivated fetal bovine serum [FBS], 100 IU/mL penicillin, 100 μg/mL streptomycin, recombinant human IL-2 [50 U/mL, PeproTech, Cranbury, NJ], recombinant human IL-15 [5 ng/mL, R & D Systems], and recombinant human IL-7 [5 ng/mL, R & D Systems]), seeded at 1.0 E6/mL, and rested overnight at 37° C. On Day 0, CD3+ T cells were washed and resuspended in fresh T cell medium and activated using ImmunoCult™ human CD3/CD28/CD2 T cell activator (5 μL/1.0 E6 CD3+ T cells, Stem Cell Technologies). On Day 1, cells were washed and resuspended in fresh complete T cell medium and transduced with lentiviral particles to express the recombinant TCR for TSC-203-A0201.

On Day 2, cells were washed and resuspended in fresh complete T cell medium and expanded until Day 5 in G-Rex 6-well plates (Wilson Wolf). On Day 5, cells were isolated using fluorescent activated cell sorting (FACS). Cells were harvested and resuspended in EasySep™ buffer (StemCell Technologies) and incubated with HLA-A*02:01/SLLQHLIGL Dextramer-PE (Immudex, 1:50 dilution) at 4° C. for 20 minutes. Anti-CD34-Alexa Fluor®488 (R & D Systems, 1:50 dilution) was added directly to cells and incubated at 4° C. for an additional 10-20 minutes. Cells were washed with EasySep™ buffer, centrifuged and resuspended in EasySep™ buffer containing DAPI (1:200 dilution). Live dextramer binding (DAPI− CD34⁺ Dextramer⁺) T cells were sorted on a MoFlo Astrios EQ cell sorter (Beckman Coulter). The isolated cells were resuspended in fresh complete T cell medium and expanded in G-Rex®10 flasks (Wilson Wolf) until Day 12, at which point the cells were frozen down in CryoStor® CS10 and stored in liquid nitrogen until used.

The process used to engineer mechanistically representative TCR-T cells used for the follow up experiments was overall similar to the process described above, with one major difference: the TCR-T cells were not isolated by FACS on day 5, and instead were expanded in complete T cell medium up to Day 14 before being frozen down.

A 96-well-based, MHC-expressing array was generated. Briefly, endogenous HLA-A/B/C were knocked out in HEK293T cells using CRISPR. Guide RNAs were designed against sequences conserved across the HLA-A, HLA-B and HLA-C loci using the multicrispr.net tool (Prykhozhij et al. (2015) PLoS One 10:30138634). The following guides were selected: CRISPR-ALL-1: CGGCTACTACAACCAGAGCG, CRISPR-ALL-2: AGATCACACTGACCTGGCAG, CRISPR-ALL-3: AGGTCAGTGTGATCTCCGCA, gRNAs were cloned into the LentiCRISPR V2 vector using BsmBI sites. HEK293T cells were transfected with plasmid guide constructs using Mirus TransIT (Mirus Bio, Madison, WI). After 7 days, MHC-negative cells were sorted using a pan-MHC antibody (BioLegend). Single cell clones were expanded, and the absence of MHC was verified by flow cytometry and Western blot.

MHC-null HEK293T cells were transduced with IncuCyte® NucLight™ Red virus (Essen BioScience). Transduced wells were sorted for NucLight™ Red expression using a Sony SH800 sorter. To generate an MHC-expressing array, MHC-null NucLight™ Red-expressing HEK 293T cells were individually transduced with the most common 110 MHCs (pHAGE-EF1a-MHC-UBC-NAT) in individual wells in 96-well plates. Transduced cells were selected with nourseothricin (400 ng/ml, GoldBio) for one week. Cells expressing the most common 110 class I MHC alleles were passaged and stored in 96-well plates as an array.

To generate the positive control for the assay, the HLA-A*02:01-expressing cells in the above array were transduced with a 90-mer construct, which contained the PRAME epitope (SLLQHLIGL), and sorted for AmCyan expression using an Invitrogen™ Bigfoot™ Spectral Cell Sorter with Sasquatch Software (SQS). Expression of individual MHC alleles in the array was verified by staining using a pan-MHC class I antibody (BioLegend).

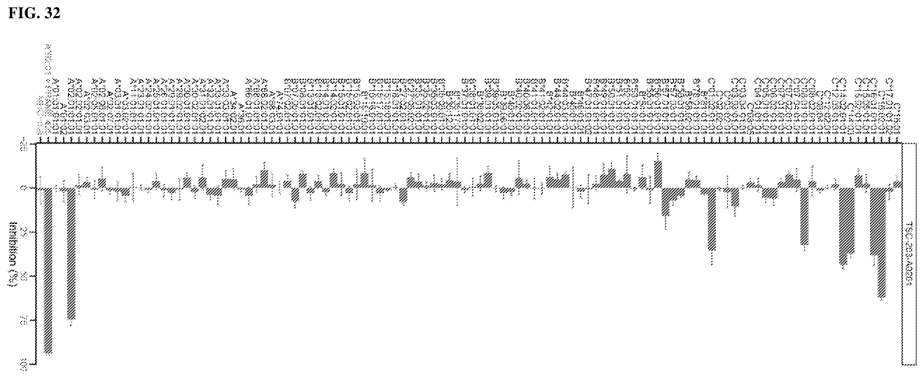

Coculture for alloreactivity profiling was set up. In brief, on day 0, mechanistically representative TSC-203-A0201 TCR-T cells were restimulated in upright T25 flasks with 1 E6 T cells, 20 E6 irradiated PBMCs, recombinant human IL-2 [50 U/mL, Peprotech], and CD3 monoclonal antibody (OKT3) [0.1 pg/mL, eBioscience]. Half the volume of media was exchanged on days 2, 4, and 5 with RPMI 1640 supplemented with 10% heat-inactivated fetal bovine serum [FBS], 100 IU/mL penicillin, 100 μg/mL streptomycin, and recombinant human IL-2. On day 5, target cells in the 96-well array were passaged and seeded in 384-well plates. On day 6, T cells were harvested and resuspended in cytokine medium containing IL-2 [100 U/mL] for the coculture. The assay was performed in triplicate. Mechanistically representative TSC-203-A0201 TCR-T cells or untransduced control T cells were added at an effector:target (E:T) ratio of 5:1 and incubated with target cells for 48 hours (FIG. 32). Target cell numbers were measured over time using an IncuCyte S3®. Cell growth inhibition at indicated timepoint of coculture on each MHC-expressing HEK293T cells in the assay was calculated as 1−(Cell doubling[Incubated with mechanistically representative TSC-203-A0201 T cells]/Cell doubling[Incubated with UTD donor T cells]). To correct for batch effect of each 384-well plate, the cell inhibition value of each well (each MHC) on each 384-well plate were subtracted by the median of cell inhibition of the wells on each 384-well plate.

Mechanistically representative TSC-203-A0201 TCR-T cells achieved complete (93.2±0.9%) cell growth inhibition on the target cells expressing HLA-A*02:01 and the PRAME 90mer containing the on-target epitope after 48 hours of coculture (FIG. 32). This condition served as a positive control for the assay confirming that the effector T cells were functionally capable of reacting to their cognate peptide/MHC target. In the absence of exogenous PRAME expression. TSC-203-A0201 TCR-T cells also showed reactivity to the HLA-A*02:01-positive monoallelic HEK293T cells. This condition further served as a positive control for the assay. In contrast, FIG. 32 shows that TSC-203-A0201 did not show significant cell growth inhibition on target cells expressing most of the 110 most common class I MHC alleles, with the exception of HEK293T cells expressing HLA alleles C*01:02, C*08:01. C*14:02, C*14:03. C*16:01, and C*16:02. The alloreactivity profiling assay was performed twice with comparable results.

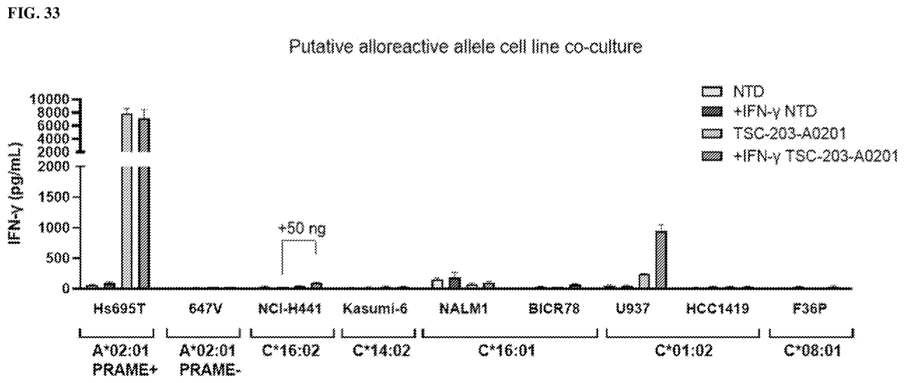

To confirm the alloreactivity profile of TSC-203-A0201 TCR-T cells, mechanistically representative TCR-T cells and donor-matched non-transduced (NTD) control T cells were cocultured with cell lines naturally expressing the putative allogeneic alleles identified during the high throughput alloreactivity profiling assay. Cancer cell lines expressing HLA-C*01:02, HLA-C*08:01, HLA-C*14:02, HLA-C*16:01, and HLA-C*16:02 were identified (Table). These cells should not normally represent targets of TSC-203-A0201 as they either lack PRAME or HLA-A*02:01, or both. These target cell lines underwent pre-treatment with 25 ng/mL IFN-γ with the goal of upregulating levels of surface HLA to maximize the sensitivity of the assay (Stark et al. (1998) Annu. Rev. Biochem, 67:227-264; Chen et al. (1986) Mol. Cell Biol, 6:1698-1705). Untreated cells or cells pre-treated with IFN-γ were then washed to eliminate exogenous IFN-γ, and were cocultured with two batches of TSC-203-A0201 or donor-matched NTD control T cells for 24 hours; IFN-γ release by the T cells was measured as a readout of T cell engagement (FIG. 33 shows representative data from one donor). Surface expression of pan HLA at the surface of the target cells was evaluated by flow cytometry to confirm the positive effect of IFN-γ pre-treatment on HLA expression. This analysis confirmed that target cells express variable levels of HLA at their surface as suggested by transcriptional data provided in Table. The data further confirmed that IFN-γ pre-treatment led to the upregulation of surface expression of these proteins in all the target cell lines used in the assay.

Coculturing with cancer cell lines and IFN-γ detection are described in more detail below. Specifically, cancer cell lines expressing the putative allogeneic HLAs for the therapeutic TCR used in TSC-203-A0201 were cultured according to vendor recommendations (Table 16) with respect to media and subculture conditions. Prior to coculture with mechanistically representative TSC-203-A0201 TCR-T cells, 8 E5 cells per well of each cell line were plated in 6-well plates in the appropriate media. Cells were either treated with 25 ng/mL IFN-γ (R & D Systems) or were left untreated; after 24 h of incubation, each line was collected and washed 3 times. A portion of cells was stained for HLA-ABC expression (BioLegend) and the remainder were plated at 5 E4/well in 96-well flat-bottom plates.

One day prior to initiating the coculture, TCR-T cells and their non-transduced (NTD) control T cell counter parts were thawed and washed to remove cryopreservation buffer. Cells were rested overnight in complete T cell medium. On the day of coculture, T cells were washed and resuspended in cytokine-free T cell medium. IE5 TSC-203-A0201 TCR-T cells or NTD control T cells were added to appropriate wells, and supernatant was collected 24 hours later and frozen at −80° C. IFN-γ production was subsequently measured in the supernatants using an automated 4-plex ELISA platform (ELLA from Protein Simple ELLA) according to the manufacturer's instructions. For coculture with monoallelic C*14:03-expressing HEK293T cells, 5 E4 cells/well were plated in flat-bottom plates, followed by addition of 1 E5 TSC-203-A0201 TCR-T cells or NTD controls for 24 hours, collection of supernatants, and quantification of IFN-γ production using the ELLA platform as above.

TABLE 16
Target cells naturally expressing putative allogeneic alleles for TSC-203-A0201
Expression as transcripts per million (TPM) of PRAME and HLA-A and HLA-C in the
different cell lines are indicated (Source: cancer cell line encyclopedia, Broad Institute)

| | Vendor/ | | HLA-A | HLA-C | PRAME |
|---|---|---|---|---|---|
| Cell line | cat # | Media | (TPM) | (TPM) | (TPM) |
| Hs695T | ATCC/ | Eagle's Minimum | A*02:01/A*02:01 | C*07:01/C*07:01 | + |
| (Positive | HTB-137 | Essential Medium + | (520.93) | (916.32) | (242.23) |
| control) | | 10% FBS | | | |
| 647V | DSMZ/ | DMEM + | A*02:01/A*02:01 | C*02:02/C*03:04 | − |
| (Negative) | ACC 414 | 15% FBS | (988.35) | (347.03) | (0.1) |
| control) | | | | | |
| NCI-H441 | ATCC/ | RPMI-1640 + | A*02:01/A*03:01 | C*16:02/C*16:02 | − |
| | HTB-174 | 10% FBS | (1129) | (227.9) | (2.86) |
| Kasumi-6 | ATCC/ | RPMI-1640 + | A*31:01/A*02:01 | C*14:02/C*03:04 | − |
| | CRL-2775 | rhGMCSF + 20% | (642.32) | (116.94) | (0.16) |
| | | FBS | | | |
| NALM1 | ATCC/ | RPMI-1640 + | A*33:03/A*02:01 | C*04:01/C*16:01 | − |
| | CRL-1567 | 15% FBS | (712.52) | (431.18) | (0.24) |
| BICR78 | Sigma | DMEM + 10% FBS + | A*29:02/A*02:01 | C*16:01/C*05:01 | − |
| | 04072111-1VL | 0.4 mg/mL | (69.14) | (39.77) | (0.01) |
| | | hydrocortisone | | | |
| U-937 | ATCC/ | RPMI-1640 + | A*03:01/A*03:01 | C*01:02/C*07:01 | − |
| | CRL-1593.2 | 10% FBS | (291.42) | (527.34) | (1.65) |
| HCC1419 | ATCC/ | RPMI-1640 + | A*24:02/A*02:01 | C*03:03/C*01:02 | − |
| | CRL-2326 | 10% FBS | (47.37) | (96.4) | (0.17) |
| F36P | Sigma | RPMI-1640 + rhIL- | A*24:02/A*33:03 | C*03:02/C*08:01 | + |
| | 99072808-1VL | 3 + 5% FBS | (181.39) | (155.27) | (28.2) |

TABLE 17
Media and Supplements

| Media | Vendor | Catalog Number |
|---|---|---|
| RPMI 1640 | Gibco | A10491-01 |
| DMEM | Gibco | 11965 |
| EMEM | Corning | 10009CV |
| LymphoONE | Takara | WK552S |
| GlutaMAX supplement | Gibco | 35050061 |
| Penicillin streptomycin | Gibco | 15149-122 |
| Heat-inactivated fetal bovine | Gibco | A3840102 |
| serum (FBS) | | |
| Heat-inactivated human serum | MP Biomedicals | 2930949 |
| OptiPRO SFM | ThermoFisher | 12309019 |
| | Scientific | |
| Recombinant human IL-3 | R&D Systems | 203-IL-010 |
| Recombinant human GM-CSF | R&D Systems | 215-GM-050 |
| Hydrocortisone | Sigma Aldrich | H0396-100MG |

Reagents

| Reagent | Vendor | Catalog Number |
|---|---|---|
| Recombinant human IL-2 | PeproTech | AF-200-02 |
| Recombinant human IL-7 | R&D Systems | 207-IL-025 |
| Recombinant human IL-15 | R&D Systems | 247-ILB-005 |
| Recombinant human IFN-γ | R&D Systems | 10067-IF-025 |
| Nourseothricin | GoldBio | N-500-1 |
| APC anti-human HLA A, B, C Antibody | BioLegend | 311410 |
| Anti-human CD34-Alexa Fluor 488 (clone: Qbend10) | R&D Systems | 310916 |
| DAPI (4′,6-diamidino-2-phenylindole) | Thermo Scientific | 62248 |
| HLA-A*02:01/SLLQHLIGL Dextramer-APC | Immudex | Custom |
| CD3 monoclonal antibody (OKT3) | eBioscience | 16-0037-81 |
| ImmunoCult ™ Human CD3/CD28/CD2 T cell activator | StemCell | 10970 |
| | Technologies | |
| Incucyte ® NucLight Red Lentivirus Reagent | Essen BioScience | 4476 |
| Mirus TransIT ®-293 Transfection Reagent | Mirus Bio | MIR2704 |
| Alt-R ® S.p. Cas9 Nuclease V3 (Lot #0000417827) | IDT | 1081059 |
| Alt-R ® CRISPR-Cas9 tracrRNA (Lot #0000415438) | IDT | 1072534 |
| StraightFrom ® Leukopak ® CD3 Microbead Kit, human | Miltenyl Biotec | 130-122-365 |
| autoMACS ® Running Buffer - MACS ® Separation Buffer | Miltenyl Biotec | 130-091-221 |
| EasySep ™ Buffer | StemCell | 20144 |
| | Technologies | |
| CryoStor ® CS10 | StemCell | 100-1061 |
| | Technologies | |
| Luna ® Universal One-Step RT-qPCR Kit | New England | E3005X |
| | BioLabs | |
| jetPRIME Transfection Reagent | Polyplus- | 114-75 |
| | transfection | |

As shown in FIG. 33, baseline levels of IFN-γ production were measured in the supernatant of NTD control T cells cocultured with the different target cell lines that were either untreated or pre-treated with IFN-γ. Whereas TSC-203-A0201 TCR-T cells produced a significantly increased amount of IFN-γ when cocultured with the positive control cell line Hs695T (>8000 μg/mL), with target cells untreated or pre-treated or with IFN-γ, the TCR-T cells failed to produce any IFN-γ above baseline when cocultured with the negative control cell line 647V and similarly with Kasumi-6, NALM1, BICR78, HCC1419, and F36P cells. This observation indicates that the therapeutic TCR did not react to the target cells expressing some of the putative allogeneic alleles (i.e., HLA-C*14:02, HLA-C*16:01, or HLA-C*08:01). However, TSC-203-A0201 TCR-T cells displayed appreciable reactivity to U937 cells (either untreated or pre-treated or with IFN-γ, producing 944 μg/mL and 242 μg/mL of IFN-γ, respectively, compared to 50.6 and 41.6 pg/mL at baseline, respectively). TSC-203-A0201 also showed low level reactivity to NCI-H441 cells, but only when the HLA levels were enhanced by IFN-γ pre-treatment. A 2.5-fold induction of IFN-γ secretion was measured with levels of IFN-γ produced reaching 98 μg/mL for TSC-203-A0201, compared to 38.5 pg/mL at baseline with NTD control T cells.

Regarding U937, these cells are HLA-A*02:01- and PRAME-negative; they are, however, positive for HLA-C*01:02 (Table). In line with high throughput data presented in FIG. 32, reactivity of TSC-203-A0201 to these cancer cells confirms that HLA-C*01:02 represents a potential allogeneic allele for the therapeutic PRAME TCR. It is worth noting that TSC-203-A0201 failed to react to HCC1419 cells which are also HLA-C*01:02-positive (FIG. 33). Altogether, although this data confirmed alloreactivity of the TCR for HLA-C*02:01, it also indicated that this reactivity only manifests itself when HLA expression is exceptionally high, as seen in U937 cancer cells. The level of engagement of TSC-203-A0201 under these conditions remains low, as it was ˜10-fold lower than when the TCR-T cells are engaging their cognate target.

Regarding NCI-H441, while these cells are positive for HLA-A*02:01, they do not express PRAME. However, the cells are homozygous for the HLA-C*16:02 allele (Table). A stimulation of IFN-γ secretion by TSC-203-A0201 was observed when the TCR-T cells were cocultured with IFN-γ pre-treated cells; no such reactivity was observed in the absence of pre-treatment. This induction remained minimal (an increase of only 50 μg/mL was quantified) but confirmed, nevertheless, that HLA-C*16:02 represents a potentially allogeneic allele for the therapeutic PRAME TCR, as originally suggested by the high throughput data presented in FIG. 32.

No other cell line presented reactivity to TSC-203-A0201, even when HLA expression had been enhanced by IFN-γ pre-treatment. These observations suggest that the therapeutic TCR is not naturally alloreactive to HLA-C*08:01, C*14:02, and C*16:01, contrary to what was originally suggested from the high throughput alloreactivity screen (FIG. 32).

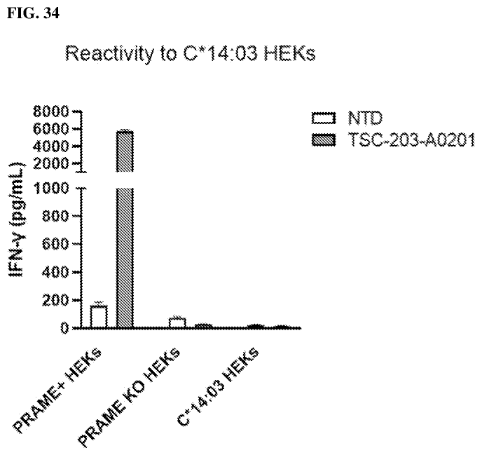

The frequency of HLA-C*14:03 allele is extremely low in the general population (<0.015% of the US population; Table 155), making sourcing cell lines naturally positive for HLA-C*14:03 for follow-up experiments difficult. To evaluate this allele for alloreactivity in follow-up experiments, HEK293T cells overexpressing the C*14:03 allele were used instead (FIG. 34).

TSC-203-A0201 TCR-T cells and their non-transduced (NTD) control counterparts were cocultured with HEK293T cells expressing PRAME and HLA-A*02:01 as a positive control. TSC-203-A0201 TCR-T cells presented a high level of IFN-γ secretion (>5000 pg/mL) while NTD control T cells only secreted baseline levels of IFN-γ (˜50 pg/mL). When cocultured with PRAME KO, HLA-A*02:01-expressing HEK293T cells used as negative control. TSC-203-A0201, similarly to NTD control T cells, showed no secretion of IFN-γ above baseline levels. Interestingly, TSC-203-A0201 TCR-T cells showed no reactivity when cocultured with HEK293T cells over-expressing HLA-C*14:03, producing identical baseline levels of IFN-γ as the NTD control T cells. This observation suggests while HLA-C*14:03 scored positive in the high throughput screen (FIG. 32), the therapeutic TCR is not naturally alloreactive to this HLA allele under the coculture conditions of the follow-up experiment.

Altogether, the data demonstrate that the high throughput, array-based alloreactivity profiling of a TCR of interest tends to over predict allogeneic alleles. The reasons for this high sensitivity derive from the experimental design of the alloreactivity profiling assay itself.

### INCORPORATION BY REFERENCE

All publications, patents, and patent applications mentioned herein are hereby incorporated by reference in their entirety as if each individual publication, patent or patent application was specifically and individually indicated to be incorporated by reference. In case of conflict, the present application, including any definitions herein, will control.

Also incorporated by reference in their entirety are any polynucleotide and polypeptide sequences which reference an accession number correlating to an entry in a public database, such as those maintained by The Institute for Genomic Research (TIGR) on the World Wide Web at tigr.org and/or the National Center for Biotechnology Information (NCBI) on the World Wide Web at ncbi.nlm.nih.gov.

### EQUIVALENTS AND SCOPE

The details of one or more embodiments encompassed by the present invention are set forth in the description above. Although representative, exemplary materials and methods have been described above, any materials and methods similar or equivalent to those described herein may be used in the practice or testing of embodiments encompassed by the present invention. Other features, objects and advantages related to the present invention are apparent from the description. Unless defined otherwise, all technical and scientific terms used herein have the same meaning as commonly understood by one of ordinary skill in the art to which the present invention belongs. In the case of conflict, the present description provided above will control.

Those skilled in the art will recognize or be able to ascertain using no more than routine experimentation, many equivalents to the specific embodiments encompassed by the present invention described herein. The scope encompassed by the present invention is not intended to be limited to the description provided herein and such equivalents are intended to be encompassed by the appended claims.

It is also noted that the term “comprising” is intended to be open and permits but does not require the inclusion of additional elements or steps. When the term “comprising” is used herein, the term “consisting of” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges may assume any specific value or subrange within the stated ranges in different embodiments encompassed by the present invention, to the tenth of the unit of the lower limit of the range, unless the context clearly dictates otherwise.

In addition, it is to be understood that any particular embodiment encompassed by the present invention that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions encompassed by the present invention (e.g., any antibiotic, therapeutic or active ingredient; any method of production; any method of use; etc.) may be excluded from any one or more claims, for any reason, whether or not related to the existence of prior art.

It is to be understood that the words which have been used are words of description rather than limitation, and that changes may be made within the purview of the appended claims without departing from the true scope and spirit encompassed by the present invention in its broader aspects.

While the present invention has been described at some length and with some particularity with respect to several described embodiments, it is not intended that it should be limited to any such particulars or embodiments or any particular embodiment, but it is to be construed with references to the appended claims so as to provide the broadest possible interpretation of such claims in view of the prior art and, therefore, to effectively encompass the intended scope encompassed by the present invention.

What is claimed is:

1. An immunogenic peptide comprising a peptide epitope selected from peptide sequences listed in Table 1.

2. An immunogenic peptide consisting of a peptide epitope selected from peptide sequences listed in Table 1.

3. The immunogenic peptide of claim 1 or 2, wherein the immunogenic peptide is derived from a PRAME protein, optionally wherein the immunogenic peptide is 8, 9, 10, 11, 12, 13, 14, 15, 16, 17, 18, 19, 20, 21, 22, 23, 24, or 25 amino acids in length.

4. The immunogenic peptide of any one of claims 1-3, wherein the immunogenic peptide is capable of eliciting an immune response against PRAME and/or PRAME-expressing cells in a subject, optionally wherein the immune response is i) a T cell response and/or a CD8+ T cell response and/or ii) selected from the group consisting of T cell expansion, cytokine release, and/or cytotoxic killing.

5. An immunogenic composition comprising at least one immunogenic peptide according to any one of claims 1-4.

6. The immunogenic composition of claim 5, further comprising an adjuvant.

7. The immunogenic composition of claim 5 or 6, wherein the immunogenic composition is capable of eliciting an immune response against PRAME and/or PRAME-expressing cells in a subject, optionally wherein the immune response is i) a T cell response and/or a CD8+ T cell response and/or ii) selected from the group consisting of T cell expansion, cytokine release, and/or cytotoxic killing.

8. A composition comprising a peptide epitope selected from peptide sequences listed in Table 1, and an MHC molecule.

9. The composition of claim 8, wherein the MHC molecule is an MHC multimer, optionally wherein the MHC multimer is a tetramer.

10. The composition of claim 8 or 9, wherein the MHC molecule is an MHC class I molecule.

11. The composition of any one of claims 9-11, wherein the MHC molecule comprises an MHC alpha chain that is an HLA serotype selected from the group consisting of HLA-A*02, HLA-A*03, HLA-A*01, HLA-A*l 1, HLA-A*24, HLA-B*07, HLA-C*07, HLA-C*01, HLA-C*02, HLA-C*03, HLA-C*04, HLA-C*05, HLA-C*06, HLA-C*08, HLA-C*12, HLA-C*14, HLA-C*15, HLA-C*16, HLA-C*17, and HLA-C*18, optionally wherein the HLA allele is selected from the group consisting of HLA-A*02:01, HLA-A*02:02, HLA-A*02:03, HLA-A*02:04, HLA-A*02:05, HLA-A*02:06, HLA-A*02:07, HLA-A*02:10, HLA-A*02:11, HLA-A*02:12, HLA-A*02:13, HLA-A*02:14, HLA-A*02:16, HLA-A*02:17, HLA-A*02:19, HLA-A*02:20, HLA-A*02:22, HLA-A*02:24, HLA-A*02:30, HLA-A*02:42, HLA-A*02:53, HLA-A*02:60, HLA-A*02:74 allele, HLA-A*03:01, HLA-A*03:02, HLA-A*03:05, HLA-A*03:07, HLA-A*01:01, HLA-A*01:02, HLA-A*01:03, HLA-A*01:16 allele, HLA-A*11:01, HLA-A*11:02, HLA-A*11:03, HLA-A*11:04, HLA-A*11:05, HLA-A*l 1:19 allele, HLA-A*24:02, HLA-A*24:03, HLA-A*24:05, HLA-A*24:07, HLA-A*24:08, HLA-A*24:10, HLA-A*24:14, HLA-A*24:17, HLA-A*24:20, HLA-A*24:22, HLA-A*24:25, HLA-A*24:26, HLA-A*24:58 allele, HLA-B*07:02, HLA-B*07:04, HLA-B*07:05, HLA-B*07:09, HLA-B*07:10, HLA-B*07:15, HLA-B*07:21, HLA-C*07:02, HLA-C*07:01, HLA-C*04:01, HLA-C*06:02, HLA-C*03:04, HLA-C*05:01, HLA-C*16:01, HLA-C*02:02, HLA-C*03:03, HLA-C*12:03, HLA-C*08:02, HLA-C*01:02, HLA-C*17:01, HLA-C*15:02, HLA-C*14:02, HLA-C*12:02, HLA-C*07:04, HLA-C*08:01, HLA-C*03:02, HLA-C*18:01, HLA-C*15:05, HLA-C*16:02, HLA-C*08:04, HLA-C*03:05, and HLA-C*14:03 allele.

12. A stable MHC-peptide complex, comprising an immunogenic peptide according to any one of claims 1-4 in the context of an MHC molecule.

13. The stable MHC-peptide complex of claim 12, wherein the MHC molecule is an MHC multimer, optionally wherein the MHC multimer is a tetramer.

14. The stable MHC-peptide complex of claim 12 or 13, wherein the MHC molecule is an MHC class I molecule.

15. The stable MHC-peptide complex of any one of claims 12-14, wherein the MHC molecule comprises an MHC alpha chain that is an HLA serotype selected from the group consisting of HLA-A*02, HLA-A*03, HLA-A*01, HLA-A*11, HLA-A*24, HLA-B*07, HLA-C*07, HLA-C*01, HLA-C*02, HLA-C*03, HLA-C*04, HLA-C*05, HLA-C*06, HLA-C*08, HLA-C*12, HLA-C*14, HLA-C*15, HLA-C*16, HLA-C*17, and HLA-C*18, optionally wherein the HLA allele is selected from the group consisting of HLA-A*02:01, HLA-A*02:02, HLA-A*02:03, HLA-A*02:04, HLA-A*02:05, HLA-A*02:06, HLA-A*02:07, HLA-A*02:10, HLA-A*02:11, HLA-A*02:12, HLA-A*02:13, HLA-A*02:14, HLA-A*02:16, HLA-A*02:17, HLA-A*02:19, HLA-A*02:20, HLA-A*02:22, HLA-A*02:24, HLA-A*02:30, HLA-A*02:42, HLA-A*02:53, HLA-A*02:60, HLA-A*02:74 allele, HLA-A*03:01, HLA-A*03:02, HLA-A*03:05, HLA-A*03:07, HLA-A*01:01, HLA-A*01:02, HLA-A*01:03, HLA-A*01:16 allele, HLA-A*l 1:01, HLA-A*11:02, HLA-A*11:03, HLA-A*11:04, HLA-A*11:05, HLA-A*11:19 allele, HLA-A*24:02, HLA-A*24:03, HLA-A*24:05, HLA-A*24:07, HLA-A*24:08, HLA-A*24:10, HLA-A*24:14, HLA-A*24:17, HLA-A*24:20, HLA-A*24:22, HLA-A*24:25, HLA-A*24:26, HLA-A*24:58 allele, HLA-B*07:02, HLA-B*07:04, HLA-B*07:05, HLA-B*07:09, HLA-B*07:10, HLA-B*07:15, HLA-B*07:21, HLA-C*07:02, HLA-C*07:01, HLA-C*04:01, HLA-C*06:02, HLA-C*03:04, HLA-C*05:01, HLA-C*16:01, HLA-C*02:02, HLA-C*03:03, HLA-C*12:03, HLA-C*08:02, HLA-C*01:02, HLA-C*17:01, HLA-C*15:02, HLA-C*14:02, HLA-C*12:02, HLA-C*07:04, HLA-C*08:01, HLA-C*03:02, HLA-C*18:01, HLA-C*15:05, HLA-C*16:02, HLA-C*08:04, HLA-C*03:05, and HLA-C*14:03 allele; optionally wherein the HLA serotype is HLA-A*02; and further optionally wherein the HLA-A*02 is HLA-A*02:01.

16. The stable MHC-peptide complex of any one of claims 12-15, wherein the peptide epitope and the MHC molecule are covalently linked and/or wherein the alpha and beta chains of the MHC molecule are covalently linked.

17. The stable MHC-peptide complex of any one of claims 12-16, wherein the stable MHC-peptide complex comprises a detectable label, optionally wherein the detectable label is a fluorophore.

18. An immunogenic composition comprising the stable MHC-peptide complex according to any one of claims 12-17, and an adjuvant.

19. An isolated nucleic acid that encodes the immunogenic peptide of according to any one of claims 1-4, or a complement thereof.

20. A vector comprising the isolated nucleic acid of claim 19.

21. A cell that a) comprises the isolated nucleic acid of claim 19, b) comprises the vector of claim 20, and/or c) produces one or more immunogenic peptides according to any one of claims 1-4 and/or presents at the cell surface one or more stable MHC-peptide complexes according to any one of claims 12-17, optionally wherein the cell is genetically engineered.

22. A device or kit comprising a) one or more immunogenic peptides according to any one of claims 1-4 and/or b) one or more stable MHC-peptide complexes according to any one of claims 12-17, said device or kit optionally comprising a reagent to detect binding of a) and/or b) to a binding protein, optionally wherein the binding protein is an antibody, an antigen-binding fragment of an antibody, a TCR, an antigen-binding fragment of a TCR, a single chain TCR (scTCR), a chimeric antigen receptor (CAR), or a fusion protein comprising a TCR and an effector domain.

23. A method of detecting T cells that bind a stable MHC-peptide complex comprising:
a) contacting a sample comprising T cells with a stable MHC-peptide complex according to any one of claims 12-17; and
b) detecting binding of T cells to the stable MHC-peptide complex, optionally further determining the percentage of stable MHC-peptide-specific T cells that bind to the stable MHC-peptide complex, optionally wherein the sample comprises peripheral blood mononuclear cells (PBMCs).

24. The method of claim 23, wherein the T cells are CD8+ T cells.

25. The method of any one of claims 22-24, wherein the detecting and/or determining is performed using fluorescence activated cell sorting (FACS), enzyme linked immunosorbent assay (ELISA), radioimmune assay (RIA), immunochemically, Western blot, or intracellular flow assay.

26. The method of any one of claims 22-25, wherein the sample comprises T cells contacted with, or suspected of having been contacted with, one or more PRAME proteins or fragments thereof.

27. A method of determining whether a T cell has had exposure to PRAME comprising:
a) incubating a cell population comprising T cells with an immunogenic peptide according to any one of claims 1-4 or a stable MHC-peptide complex according to any one of claims 12-17; and
b) detecting the presence or level of reactivity,
wherein the presence of or a higher level of reactivity compared to a control level indicates that the T cell has had exposure to PRAME, optionally wherein the cell population comprising T cells is obtained from a subject.

28. A method for predicting the clinical outcome of a subject afflicted with a disorder characterized by PRAME expression comprising:
a) determining the presence or level of reactivity between T cells obtained from the subject and one more immunogenic peptides according to any one of claims 1-4 or one or more stable MHC-peptide complexes according to any one of claims 12-17; and
b) comparing the presence or level of reactivity to that from a control, wherein the control is obtained from a subject having a good clinical outcome,
wherein the presence or a higher level of reactivity in the subject sample as compared to the control indicates that the subject has a good clinical outcome.

29. A method of assessing the efficacy of a therapy for a disorder characterized by PRAME expression comprising:
a) determining the presence or level of reactivity between T cells obtained from the subject and one more immunogenic peptides according to any one of claims 1-4 or one or more stable MHC-peptide complexes according to any one of claims 12-17, in a first sample obtained from the subject prior to providing at least a portion of the therapy to the subject, and
b) determining the presence or level of reactivity between the one more immunogenic peptides according to any one of claims 1-4, or the one or more stable MHC-peptide complexes according to any one of claims 12-17, and T cells obtained from the subject present in a second sample obtained from the subject following provision of the therapy to the subject,
wherein the presence or a higher level of reactivity in the second sample, relative to the first sample, is an indication that the therapy is efficacious for treating the disorder characterized by PRAME expression in the subject.

30. The method of any one of claims 27-29, wherein the level of reactivity is indicated by a) the presence of binding and/or b) T cell activation and/or effector function, optionally wherein the T cell activation or effector function is T cell proliferation, killing, or cytokine release.

31. The method of any one of claims 27-30, further comprising repeating steps a) and b) at a subsequent point in time, optionally wherein the subject has undergone treatment to ameliorate the disorder characterized by PRAME expression between the first point in time and the subsequent point in time.

32. The method of any one of claims 27-31, wherein the T cell binding, activation, and/or effector function is detected using fluorescence activated cell sorting (FACS), enzyme linked immunosorbent assay (ELISA), radioimmune assay (RIA), immunochemically, Western blot, or intracellular flow assay.

33. The method of any one of claims 27-32, wherein the control level is a reference number.

34. The method of any one of claims 27-33, wherein the control level is a level of a subject without the disorder characterized by PRAME expression.

35. A method of preventing and/or treating a disorder characterized by PRAME expression in a subject comprising administering to the subject a therapeutically effective amount of a composition according to any one of claims 1-22.

36. A method of identifying a peptide-binding molecule, or antigen-binding fragment thereof, that binds to a peptide epitope selected from the peptide sequences listed in Table 1 comprising:
a) providing a cell presenting a peptide epitope selected from the peptide sequences listed in Table 1 in the context of an MHC molecule on the surface of the cell;
b) determining binding of a plurality of candidate peptide-binding molecules or antigen-binding fragments thereof to the peptide epitope in the context of the MHC molecule on the cell; and
c) identifying one or more peptide-binding molecules or antigen-binding fragments thereof that bind to the peptide epitope in the context of the MHC molecule.

37. The method of claim 36, wherein the step a) comprises contacting the MHC molecule on the surface of the cell with a peptide epitope selected from the peptide sequences listed in Table 1.

38. The method of claim 36, wherein the step a) comprises expressing the peptide epitope selected from the peptide sequences listed in Table 1 in the cell using a vector comprising a heterologous sequence encoding the peptide epitope.

39. A method of identifying a peptide-binding molecule or antigen-binding fragment thereof that binds to a peptide epitope selected from the peptide sequences listed in Table 1 comprising:
a) providing a peptide epitope either alone or in a stable MHC-peptide complex, comprising a peptide epitope selected from the peptide sequences listed in Table 1, either alone or in the context of an MHC molecule;
b) determining binding of a plurality of candidate peptide-binding molecules or antigen-binding fragments thereof to the peptide or stable MHC-peptide complex; and
c) identifying one or more peptide-binding molecules or antigen-binding fragments thereof that bind to the peptide epitope or the stable MHC-peptide complex, optionally wherein the MHC or MHC-peptide complex is as according to any one of claims 8-17.

40. The method of claim 39, wherein the plurality of candidate peptide binding molecules comprises an antibody, an antigen-binding fragment of an antibody, a TCR, an antigen-binding fragment of a TCR, a single chain TCR (scTCR), a chimeric antigen receptor (CAR), or a fusion protein comprising a TCR and an effector domain.

41. The method of claim 39 or 40, wherein the plurality of candidate peptide binding molecules comprises at least 2, 5, 10, 100, 10³, 10⁴, 10⁵, 10⁶, 10⁷, 10⁸, 10⁹, or more, different candidate peptide binding molecules.

42. The method of any one of claims 39-41, wherein the plurality of candidate peptide binding molecules comprises one or more candidate peptide binding molecules that are obtained from a sample from a subject or a population of subjects; or the plurality of candidate peptide binding molecules comprises one or more candidate peptide binding molecules that comprise mutations in a parent scaffold peptide binding molecule obtained from a sample from a subject.

43. The method of claim 42, wherein the subject or population of subjects are a) not afflicted with a disorder characterized by PRAME expression and/or have recovered from a disorder characterized by PRAME expression, or b) are afflicted with a disorder characterized by PRAME expression.

44. The method of claim 42 or 43, wherein the subject or population of subjects has been administered a composition according to any one of claims 1-22.

45. The method of any one of claims 42-44, wherein the subject is an animal model of a disorder characterized by PRAME expression and/or a mammal, optionally wherein the mammal is a human, a primate, or a rodent.

46. The method of any one of claims 42-45, wherein the subject is an animal model of a disorder characterized by PRAME expression, an HLA-transgenic mouse, and/or a human TCR transgenic mouse.

47. The method of any one of claims 42-46, wherein the sample comprises peripheral blood mononuclear cells (PBMCs), T cells, and/or CD8+ memory T cells.

48. The peptide-binding molecule or antigen-binding fragment thereof identified according to any one of claims 39-48, optionally wherein the peptide-binding molecule or antigen-binding fragment thereof is an antibody, an antigen-binding fragment of an antibody, a TCR, an antigen-binding fragment of a TCR, a single chain TCR (scTCR), a chimeric antigen receptor (CAR), or a fusion protein comprising a TCR and an effector domain.

49. A method of treating a disorder characterized by PRAME expression in a subject comprising administering to the subject a therapeutically effective amount of genetically engineered T cells that express a peptide-binding molecule or antigen-binding fragment thereof that i) binds to a peptide epitope selected from the sequences listed in Table 1, ii) is identified according to the method according to any one of claims 39-48, and/or iii) binds to a stable MHC-peptide complex comprising a peptide epitopes selected from the sequences listed in Table 1 in the context of an MHC molecule, optionally wherein the peptide-binding molecule or antigen-binding fragment thereof is an antibody, an antigen-binding fragment of an antibody, a TCR, an antigen-binding fragment of a TCR, a single chain TCR (scTCR), a chimeric antigen receptor (CAR), or a fusion protein comprising a TCR and an effector domain, optionally wherein the MHC or MHC-peptide complex is as according to any one of claims 8-17.

50. The method of claim 49, wherein the T cells are isolated from a) the subject, b) a donor not afflicted with the disorder characterized by PRAME expression, or c) a donor recovered from a disorder characterized by PRAME expression.

51. A method of treating a disorder characterized by PRAME expression in a subject comprising transfusing antigen-specific T cells to the subject, wherein the antigen-specific T cells are generated by:
a) stimulating immune cells from a subject with a composition according to any one of claims 1-22; and
b) expanding antigen-specific T cells in vitro or ex vivo, optionally i) isolating immune cells from the subject before stimulating the immune cells and/or ii) wherein the immune cells comprise PBMCs, T cells, CD8+ T cells, naive T cells, central memory T cells, and/or effector memory T cells.

52. The method of claim 51, wherein the agents are placed in contact under conditions and for a time suitable for the formation of at least one immune complex between the peptide epitope, immunogenic peptide, stable MHC-peptide complex, T cell receptor, and/or immune cells.

53. The method of claim 51 or 52, wherein the peptide epitope, immunogenic peptide, stable MHC-peptide complex, and/or T cell receptor are expressed by cells and the cells are expanded and/or isolated during one or more steps.

54. The method of any one of claims 23-53, wherein the disorder characterized by PRAME expression is a cancer or relapse thereof, optionally wherein the cancer is selected from the group consisting of melanoma, head & neck cancer, lung cancer, leukemia, ovarian cancer, a renal cell carcinoma (RCC), a breast carcinoma, a cervix carcinoma, or a colon carcinoma, a sarcoma, and a neuroblastoma.

55. The method of any one of claims 23-54, wherein the subject is an animal model of a disorder characterized by PRAME expression and/or a mammal, optionally wherein the mammal is a human, a primate, or a rodent.

56. A binding protein that binds a polypeptide comprising an immunogenic peptide sequence according to any one of claims 1-4, an immunogenic peptide according to any one of claims 1-4, and/or the stable MHC-peptide complex according to any one of claims 12-17, optionally wherein the binding protein is an antibody, an antigen-binding fragment of an antibody, a TCR, an antigen-binding fragment of a TCR, a single chain TCR (scTCR), a chimeric antigen receptor (CAR), or a fusion protein comprising a TCR and an effector domain.

57. The binding protein of claim 56 comprising:
a) a T cell receptor (TCR) alpha chain CDR sequence with at least about 80% identity to a TCR alpha chain CDR sequence selected from the group consisting of TCR alpha chain CDR sequences listed in Table 2; and/or
b) a TCR beta chain CDR sequence with at least about 80% identity to a TCR beta chain CDR sequence selected from the group consisting of TCR beta chain CDR sequences listed in Table 2, wherein the binding protein is capable of binding to a PRAME immunogenic peptide-MHC (pMHC) complex, optionally wherein the binding affinity has a K_{d} less than or equal to about 5×10⁻⁴ M.

58. The binding protein of claim 56 comprising:
a) a TCR alpha chain variable (V_{α}) domain sequence with at least about 80% identity to a TCR V_{α} domain sequence selected from the group consisting of TCR V_{α} domain sequences listed in Table 2; and/or
b) a TCR beta chain variable (V_{β}) domain sequence with at least about 80% identity to a TCR V_{β} domain sequence selected from the group consisting of TCR V_{β} domain sequences listed in Table 2, wherein the binding protein is capable of binding to a PRAME immunogenic peptide-MHC (pMHC) complex, optionally wherein the binding affinity has a K_{d} less than or equal to about 5×10⁻⁴ M.

59. The binding protein of claim 56 comprising:
a) a TCR alpha chain sequence with at least about 80% identity to a TCR alpha chain sequence selected from the group consisting of TCR alpha chain sequences listed in Table 2; and/or
b) a TCR beta chain sequence with at least about 80% identity to a TCR beta chain sequence selected from the group consisting of TCR beta chain sequences listed in Table 2, wherein the binding protein is capable of binding to a PRAME immunogenic peptide-MHC (pMHC) complex, optionally wherein the binding affinity has a K_{d} less than or equal to about 5×10−4 M.

60. The binding protein of claim 56 comprising:
a) a TCR alpha chain CDR sequence selected from the group consisting of TCR alpha chain CDR sequences listed in Table 2; and/or
b) a TCR beta chain CDR sequence selected from the group consisting of TCR beta chain CDR sequences listed in Table 2, wherein the binding protein is capable of binding to a PRAME immunogenic peptide-MHC (pMHC) complex, optionally wherein the binding affinity has a K_{d} less than or equal to about 5×10⁻⁴ M.

61. The binding protein of claim 56 comprising:
a) a TCR alpha chain variable (V_{α}) domain sequence selected from the group consisting of TCR V_{α} domain sequences listed in Table 2; and/or
b) a TCR beta chain variable (V_{β}) domain sequence selected from the group consisting of TCR V_{β} domain sequences listed in Table 2, wherein the binding protein is capable of binding to a PRAME immunogenic peptide-MHC (pMHC) complex, optionally wherein the binding affinity has a K_{d} less than or equal to about 5×10⁻⁴ M.

62. The binding protein of claim 56 comprising:
a) a TCR alpha chain sequence selected from the group consisting of TCR alpha chain sequences listed in Table 2; and/or
b) a TCR beta chain sequence selected from the group consisting of TCR beta chain sequences listed in Table 2, wherein the binding protein is capable of binding to a PRAME immunogenic peptide-MHC (pMHC) complex, optionally wherein the binding affinity has a K_{d} less than or equal to about 5×10⁻⁴ M.

63. The binding protein of any one of claims 56-62, wherein 1) the TCR alpha chain CDR, TCR V_{α} domain, and/or TCR alpha chain is encoded by a TRAV, TRAJ, and/or TRAC gene or fragment thereof selected from the group of TRAV, TRAJ, and TRAC genes listed in Table 2, and/or 2) the TCR beta chain CDR, TCR V_{β} domain, and/or TCR beta chain is encoded by a TRBV, TRBJ, and/or TRBC gene or fragment thereof selected from the group of TRBV, TRBJ, and TRBC genes listed in Table 2, and/or 3) each CDR of the binding protein has up to five amino acid substitutions, insertions, deletions, or a combination thereof as compared to the cognate reference CDR sequence listed in Table 2.

64. The binding protein of any one of claims 56-63, wherein the binding protein is chimeric, humanized, or human.

65. The binding protein of any one of claims 56-64, wherein the binding protein comprises a binding domain having a transmembrane domain, and an effector domain that is intracellular.

66. The binding protein of any one of claims 56-65, wherein the TCR alpha chain and the TCR beta chain are covalently linked, optionally wherein the TCR alpha chain and the TCR beta chain are covalently linked through a linker peptide.

67. The binding protein of any one of claims 56-66, wherein the TCR alpha chain and/or the TCR beta chain are covalently linked to a moiety, optionally wherein the covalently linked moiety comprises an affinity tag or a label.

68. The binding protein of claim 67, wherein the affinity tag is selected from the group consisting of aCD34 enrichment tag, glutathione-S-transferase (GST), calmodulin binding protein (CBP), protein C tag, Myc tag, HaloTag, HA tag, Flag tag, His tag, biotin tag, and V5 tag, and/or wherein the label is a fluorescent protein.

69. The binding protein of any one of claims 56-68, wherein the covalently linked moiety is selected from the group consisting of an inflammatory agent, cytokine, toxin, cytotoxic molecule, radioactive isotope, or antibody or antigen-binding fragment thereof.

70. The binding protein of any one of claims 56-69, wherein the binding protein binds to the pMHC complex on a cell surface.

71. The binding protein of any one of claims 56-70, wherein the MHC or MHC-peptide complex is as according to any one of claims 8-17.

72. The binding protein of any one of claims 56-71, wherein binding of the binding protein to the PRAME peptide-MHC (pMHC) complex elicits an immune response, optionally wherein the immune response is i) a T cell response and/or a CD8+ T cell response and/or ii) selected from the group consisting of T cell expansion, cytokine release, and/or cytotoxic killing.

73. The binding protein of any one of claims 56-72, wherein the binding protein is capable of specifically and/or selectively binding to the PRAME immunogenic peptide-MHC (pMHC) complex with a K_{d} less than or equal to about 1×10⁻⁴ M, less than or equal to about 5×10⁻⁵ M, less than or equal to about 1×10⁻⁵ M, less than or equal to about 5×10⁻⁶ M, less than or equal to about 1×10⁻⁶ M, less than or equal to about 5×10⁻⁷ M, less than or equal to about 1×10⁻⁷ M, less than or equal to about 5×10⁻⁸ M, less than or equal to about 1×10⁻⁸ M, less than or equal to about 5×10⁻⁹ M, less than or equal to about 1×10⁻⁹ M, less than or equal to about 5×10⁻¹⁰ M, less than or equal to about 1×10⁻¹⁰ M, less than or equal to about 5×10⁻¹¹ M, less than or equal to about 1×10⁻¹¹ M, less than or equal to about 5×10⁻¹² M, or less than or equal to about 1×10⁻¹² M.

74. The binding protein of any one of claims 56-73, wherein the binding protein has a higher binding affinity to the peptide-MHC (pMHC) than does a known T-cell receptor, optionally wherein the higher binding affinity is at least 1.05-fold higher.

75. The binding protein of any one of claims 56-74, wherein the binding protein induces higher T cell expansion, cytokine release, and/or cytotoxic killing than does a known T-cell receptor when contacted with target cells with a heterozygous expression of PRAME, optionally wherein the induction is at least 1.05-fold higher.

76. The binding protein of claim 75, wherein the cytotoxic killing is a target cancer cell.

77. The binding protein of claim 76, wherein the cancer is selected from the group consisting of melanoma, head & neck cancer, lung cancer, leukemia, ovarian cancer, a renal cell carcinoma (RCC), a breast carcinoma, a cervix carcinoma, or a colon carcinoma, a sarcoma, and a neuroblastoma.

78. The binding protein of any one of claims 56-77, wherein the binding protein does not bind to a pMHC complex comprising a PLA2G4E, EFNA1, and/or SLC26A1 peptide epitope.

79. A TCR alpha chain and/or beta chain selected from the group consisting of TCR alpha chain and beta chain sequences listed in Table 2.

80. An isolated nucleic acid molecule i) that hybridizes, under stringent conditions, with the complement of a nucleic acid encoding a polypeptide selected from the group consisting of polypeptide sequences listed in Table 2, ii) a sequence with at least about 80% homology to a nucleic acid encoding a polypeptide selected from the group consisting of the polypeptide sequences listed in Table 2, and/or iii) ii) a sequence with at least about 80% homology to a nucleic acid encoding listed in Table 2, optionally wherein the isolated nucleic acid molecule comprises 1) a TRAV, TRAJ, and/or TRAC gene or fragment thereof selected from the group of TRAV, TRAJ, and TRAC genes listed in Table 2 and/or 2) a TRBV, TRBJ, and/or TRBC gene or fragment thereof selected from the group of TRBV, TRBJ, and TRBC genes listed in Table 2.

81. The isolated nucleic acid of claim 80, wherein the nucleic acid is codon optimized for expression in a host cell.

82. A vector comprising the isolated nucleic acid of claim 80 or 81, optionally wherein i) the vector is a cloning vector, expression vector, or viral vector and/or ii) the vector comprises a vector sequence listed in Table 3.

83. The vector of claim 82, wherein the vector further comprises a nucleic acid sequence encoding CD8α, CD8β, a dominant negative TGFβ receptor II (DN-TGFβRII), selectable protein marker, optionally wherein the selectable protein marker is dihydrofolate reductase (DHFR).

84. The vector of claim 83, wherein the nucleic acid sequence encoding CD8α, CD8β, the DN-TGFβRII, and/or the selectable protein marker is operably linked to a nucleic acid encoding a tag.

85. The vector of claim 83 or 84, wherein the nucleic acid encoding a tag is at the 5′ upstream of the nucleic acid sequence encoding CD8α, CD8β, the DN-TGFβRII, and/or the selectable protein marker such that the tag is fused to the N-terminus of CD8α, CD8β, the DN-TGFβRII, and/or the selectable protein marker.

86. The vector of claim 84 or 85, wherein the tag is a CD34 enrichment tag.

87. The nucleic acid or vector of any one of claims 80-86, wherein the nucleic acid sequence encoding TCRα, TCRβ, CD8α, CD8β, the DN-TGFβRII, and/or the selectable protein marker are interconnected with an internal ribosome entry site or a nucleic acid sequence encoding a self-cleaving peptide.

88. The nucleic acid or vector of claim 87, wherein the self-cleaving peptide is P2A, E2A, F2A or T2A.

89. A host cell which comprises the isolated nucleic acid of claim 80 or 81, comprises the vector according to any one of claims 82-88, and/or expresses the binding protein according to any one of claims 56-78, optionally wherein the cell is genetically engineered.

90. The host cell of claim 89, wherein the host cell comprises a chromosomal gene knockout of a TCR gene, an HLA gene, or both.

91. The host cell of claim 89 or 90, wherein the host cell comprises a knockout of an HLA gene selected from an α1 macroglobulin gene, α2 macroglobulin gene, α3 macroglobulin gene, β1 microglobulin gene, β2 microglobulin gene, and combinations thereof.

92. The host cell of any one of claims 89-91, wherein the host cell comprises a knockout of a TCR gene selected from a TCR α variable region gene, TCR β variable region gene, TCR constant region gene, and combinations thereof.

93. The host cell of any one of claims 89-92, wherein the host cell expresses CD8α, CD8β, a DN-TGFβRII, and/or a selectable protein marker, optionally wherein the selectable protein marker is DHFR, further optionally wherein the CD8α, CD8β, the DN-TGFβRII, and/or the selectable protein marker is fused to a CD34 enrichment tag.

94. The host cell of claim 93, wherein host cells are enriched using the CD34 enrichment tag.

95. The host cell of any one of claims 89-94, wherein the host cell is a hematopoietic progenitor cell, peripheral blood mononuclear cell (PBMC), cord blood cell, or immune cell.

96. The host cell of claim 95, wherein the immune cell is a T cell, cytotoxic lymphocyte, cytotoxic lymphocyte precursor cell, cytotoxic lymphocyte progenitor cell, cytotoxic lymphocyte stem cell, CD4⁺ T cell, CD8⁺ T cell, CD4/CD8 double negative T cell, gamma delta (γδ) T cell, natural killer (NK) cell, NK-T cell, dendritic cell, or a combination thereof.

97. The host cell of any one of claims 89-96, wherein the T cell is a naive T cell, central memory T cell, effector memory T cell, or a combination thereof.

98. The host cell of any one of claims 89-97, wherein the T cell is a primary T cell or a cell of a T cell line.

99. The host cell of any one of claims 89-98, wherein the T cell does not express or has a lower surface expression of an endogenous TCR.

100. The host cell of any one of claims 89-99, wherein the host cell is capable of producing a cytokine or a cytotoxic molecule when contacted with a target cell that comprises a peptide-MHC (pMHC) complex comprising a PRAME peptide epitope in the context of an MHC molecule.

101. The host cell of claim 100, wherein the host cell is contacted with the target cell in vitro, ex vivo, or in vivo.

102. The host cell of claim 100 or 101, wherein the cytokine is TNF-α, IL-2, and/or IFN-γ.

103. The host cell of any one of claims 89-102, wherein the cytotoxic molecule is perforins and/or granzymes, optionally wherein the cytotoxic molecule is granzyme B.

104. The host cell of any one of claims 89-103, wherein the host cell is capable of producing a higher level of cytokine or a cytotoxic molecule when contacted with a target cell with a heterozygous expression of PRAME.

105. The host cell of claim 104, wherein the host cell is capable of producing an at least 1.05-fold higher level of cytokine or a cytotoxic molecule.

106. The host cell of any one of claims 89-103 wherein the host cell is capable of killing a target cell that comprises a peptide-MHC (pMHC) complex comprising the PRAME peptide epitope in the context of an MHC molecule.

107. The host cell of claim 106, wherein the killing is determined by a killing assay.

108. The host cell of claim 106 or 107, wherein the ratio of the host cell and the target cell in the killing assay is from 20:1 to 1:4.

109. The host cell of any one of claims 106-108, wherein the target cell is a target cell pulsed with 1 μg/mL to 50 μg/mL of PRAME peptide, optionally wherein the target cell is a cell monoallelic for an MHC matched to the PRAME peptide.

110. The host cell of any one of claims 106-109, wherein the host cell is capable of killing a higher number of target cells when contacted with target cells with a heterozygous expression of PRAME, optionally wherein the cell killing is at least 1.05-fold higher.

111. The host cell of any one of claims 89-110, wherein the target cell is cell line or a primary cell, optionally wherein the target cell is selected from the group consisting of a HEK293 derived cell line, a cancer cell line, a primary cancer cell, a transformed cell line, and an immortalized cell line; and further optionally wherein the cell line is Hs695T, A375, or NCI-H1563.

112. The host cell of any one of claims 89-111, wherein the PRAME immunogenic peptide is as according to any one of claims 1-4 and/or wherein the MHC or MHC-peptide complex is as according to any one of claims 8-17.

113. The host cell of any one of claims 89-112, wherein the host cell does not induce T cell expansion, cytokine release, or cytotoxic killing when contacted with a target cell that comprises a peptide-MHC (pMHC) complex comprising a PLA2G4E, EFNA1, and/or SLC26A1 peptide epitope.

114. The host cell of any one of claims 89-113, wherein the host cell does not express PRAME antigen, is not recognized by a binding protein of any one of claims 56-78, is not of serotype HLA-A*02, and/or does not express an HLA-A*02 allele.

115. A population of host cells according to any one of claims 89-114.

116. A composition comprising a) a binding protein according to any one of claims 56-77, b) an isolated nucleic acid according to claim 80 or 81, c) a vector according to any one of claims 82-88, d) a host cell according to any one of claims 89-114, and/or e) a population of host cells according to claim 115, and a carrier.

117. A device or kit comprising a) a binding protein according to any one of claims 56-77, b) an isolated nucleic acid according to claim 80 or 81, c) a vector according to any one of claims 82-88, d) a host cell according to any one of claims 89-114, and/or e) a population of host cells according to claim 115, said device or kit optionally comprising a reagent to detect binding of a), d) and/or e) to a pMHC complex.

118. A method of producing a binding protein according to any one of claims 56-77, wherein the method comprises the steps of: (i) culturing a transformed host cell which has been transformed by a nucleic acid comprising a sequence encoding a binding protein according to any one of claims 56-77 under conditions suitable to allow expression of said binding protein; and (ii) recovering the expressed binding protein.

119. A method of producing a host cell expressing a binding protein according to any one of claims 56-77, wherein the method comprises the steps of: (i) introducing a nucleic acid comprising a sequence encoding a binding protein according to any one of claims 56-77 into the host cell; and (ii) culturing the transformed host cell under conditions suitable to allow expression of said binding protein.

120. A method of detecting the presence or absence of a PRAME antigen and/or a cell expressing PRAME, optionally wherein the cell is a hyperproliferative cell, comprising detecting the presence or absence of said PRAME antigen in a sample by use of at least one binding protein according to any one of claims 56-77, at least one host cell according to any one of claims 89-114, or a population of host cells according to claim 115, wherein detection of the PRAME antigen is indicative of the presence of a PRAME antigen and/or cell expressing PRAME.

121. The method of claim 120, wherein the at least one binding protein, or the at least one host cell, forms a complex with the PRAME peptide in the context of an MHC molecule, and the complex is detected in the form of fluorescence activated cell sorting (FACS), enzyme linked immunosorbent assay (ELISA), radioimmune assay (RIA), immunochemically, Western blot, or intracellular flow assay.

122. The method of claim 120 or 121, further comprising obtaining the sample from a subject.

123. A method of detecting the level of a disorder characterized by PRAME expression in a subject, comprising:
a) contacting a sample obtained from the subject with at least one binding protein according to any one of claims 56-77, at least one host cell according to any one of claims 89-114, or a population of host cells according to claim 115; and
b) detecting the level of reactivity,
wherein the presence or a higher level of reactivity compared to a control level indicates the level of the disorder characterized by PRAME expression in the subject.

124. The method of claim 123, wherein the control level is a reference number.

125. The method of claim 123 or 124, wherein the control level is a level from a subject without the disorder characterized by PRAME expression.

126. A method for monitoring the progression of a disorder characterized by PRAME expression in a subject, the method comprising:
a) detecting in a subject sample the presence or level of reactivity between a sample obtained from the subject and at least one binding protein according to any one of claims 56-77, at least one host cell according to any one of claims 89-114, or a population of host cells according to claim 115;
b) repeating step a) at a subsequent point in time; and
c) comparing the level of PRAME or the cell of interest expressing PRAME detected in steps a) and b) to monitor the progression of the disorder characterized by PRAME expression in the subject, wherein an absent or reduced PRAME level or the cell of interest expressing PRAME detected in step b) compared to step a) indicates an inhibited progression of the disorder characterized by PRAME expression in the subject and a presence or increased PRAME level or the cell of interest expressing PRAME detected in step b) compared to step a) indicates a progression of the disorder characterized by PRAME expression in the subject.

127. The method of claim 126, wherein between the first point in time and the subsequent point in time, the subject has undergone treatment to treat the disorder characterized by PRAME expression.

128. A method for predicting the clinical outcome of a subject afflicted with a disorder characterized by PRAME expression comprising:
a) determining the presence or level of reactivity between a sample obtained from the subject and at least one binding protein according to any one of claims 56-77, at least one host cell according to any one of claims 89-114, or a population of host cells according to claim 115; and
b) comparing the presence or level of reactivity to that from a control, wherein the control is obtained from a subject having a good clinical outcome;
wherein the absence or a reduced level of reactivity in the subject sample as compared to the control indicates that the subject has a good clinical outcome.

129. A method of assessing the efficacy of a therapy for a disorder characterized by PRAME expression comprising:
a) determining the presence or level of reactivity between a sample obtained from the subject and at least one binding protein according to any one of claims 56-77, at least one host cell according to any one of claims 89-114, or a population of host cells according to claim 115, in a first sample obtained from the subject prior to providing at least a portion of the therapy for the disorder characterized by PRAME expression to the subject, and
b) determining the presence or level of reactivity between a sample obtained from the subject and at least one binding protein according to any one of claims 56-77, at least one host cell according to any one of claims 89-114, or a population of host cells according to claim 115, in a second sample obtained from the subject following provision of the therapy for the disorder characterized by PRAME expression,
wherein the absence or a reduced level of reactivity in the second sample, relative to the first sample, is an indication that the therapy is efficacious for treating the disorder characterized by PRAME expression in the subject, and wherein the presence or an increased level of reactivity in the second sample, relative to the first sample, is an indication that the therapy is not efficacious for treating the disorder characterized by PRAME expression in the subject.

130. The method of any one of claims 120-129, wherein the level of reactivity is indicated by a) the presence of binding and/or b) T cell activation and/or effector function, optionally wherein the T cell activation or effector function is T cell proliferation, killing, or cytokine release.

131. The method of any one of claims 120-130, wherein the T cell binding, activation, and/or effector function is detected using fluorescence activated cell sorting (FACS), enzyme linked immunosorbent assay (ELISA), radioimmune assay (RIA), immunochemically, Western blot, or intracellular flow assay.

132. A method of preventing and/or treating a disorder characterized by PRAME expression comprising contacting target cells expressing PRAME with a therapeutically effective amount of a composition comprising cells expressing at least one binding protein according to any one of claims 56-77, optionally wherein the composition is administered to a subject.

133. The method of any one of claims 49-55 and 132, wherein the cell is an allogeneic cell, syngeneic cell, or autologous cell.

134. The method of any one of claims 49-55, 132, and 133, wherein the cell is a host cell according to any one of claims 89-114 or a population of host cells according to claim 115.

135. The method of any one of claims 49-55 and 132-134, wherein the target cell is a cancer cell expressing PRAME.

136. The method of any one of claims 49-55 and 132-135, wherein the composition further comprises a pharmaceutically acceptable carrier.

137. The method of any one of claims 49-55 and 132-136, wherein the composition induces an immune response against the target cell expressing PRAME in the subject.

138. The method of any one of claims 49-55 and 132-137, wherein the composition induces an antigen-specific T cell immune response against the target cell expressing PRAME in the subject.

139. The method of any one of claims 49-55 and 132-138, wherein the antigen-specific T cell immune response comprises at least one of a CD4⁺ helper T lymphocyte (Th) response and a CD8+ cytotoxic T lymphocyte (CTL) response.

140. The method of any one of claims 49-55 and 132-139, further comprising administering at least one additional treatment for the disorder characterized by PRAME expression, optionally wherein the at least one additional treatment for the disorder characterized by PRAME expression is administered concurrently or sequentially with the composition.

141. The method of any one of claims 132-140, wherein the disorder characterized by PRAME expression is a cancer or relapse thereof, optionally wherein the cancer is selected from the group consisting of melanoma, head & neck cancer, lung cancer, leukemia, ovarian cancer, a renal cell carcinoma (RCC), a breast carcinoma, a cervix carcinoma, or a colon carcinoma, a sarcoma, and a neuroblastoma.

142. The method of any one of claims 132-141, wherein the subject is an animal model of a disorder characterized by PRAME expression and/or a mammal, optionally wherein the mammal is a human, a primate, or a rodent.
